SEQUENCE LISTING (10 sequences)

SEQ ID NO: 1 - DNA, 1401 bp, Homo Sapiens
atggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 2 - protein, 466 aa, Homo Sapiens
MAMDVTRSQAQTALTLVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

SEQ ID NO: 3 - protein, 465 aa, Homo Sapiens [sequence omitted]

SEQ ID NO: 4 - DNA, 1368 bp, Homo Sapiens
atggccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 5 - protein, 455 aa, Homo Sapiens
MALTLVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

SEQ ID NO: 6 - DNA, 39 bp, Artificial Sequence
  Primer
gtcacaaggagccagaagcttatggcctgactctggtag

SEQ ID NO: 7 - DNA, 28 bp, Artificial Sequence
  Primer
gaagccccacgacattgttcccttctgc

SEQ ID NO: 8 - protein, 451 aa, Homo Sapiens
MVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

SEQ ID NO: 9 - DNA, 38 bp, Artificial Sequence
  Primer
ccaggcccagacagccaagcttatggtagagcagatcc

SEQ ID NO: 10 - DNA, 28 bp, Artificial Sequence
  Primer
gaagccccacgacattgttcccttctgc

(19) United States
(12) Patent Application Publication
(10) Pub. No.: US 2007/0231874 A1
(43) Pub. Date: Oct. 4, 2007

(54) Crystal of glucokinase protein, and method for drug design using the crystal

(76) Inventors: Kenji Kamata, Chiba (JP); Yasufumi Nagata, Ibaraki (JP); Toshiharu Iwama, Ibaraki (JP)

Correspondence Address: MERCK AND CO., INC, P O BOX 2000, RAHWAY, NJ, 07065-0907, US

(21) Appl. No.: 10/514,928

(22) PCT Filed: May 15, 2003
(86) PCT No.: PCT/JP03/06054

§ 371 Date: Nov. 12, 2004

(30) Foreign Application Priority Data
May 16, 2002 (JP) 2002-142232

(51) Int. Cl. C12N 9/12; G06F 19/00; G01N 33/48; G01N 33/50

(52) U.S. Cl. 435/194; 702/19

(57) ABSTRACT

Glucokinase is crystallized, the three-dimensional structure thereof is analyzed, and then a compound to be bonded to glucokinase is designed on the basis of the coordinate for the resulting three-dimensional structure. Specifically, glucokinase is freed of a part of amino acid residues being on the N-terminal side thereof, to thereby crystallize it, and the three-dimensional structure of the resulting crystal is elucidated through the X-ray crystallographic analysis thereof.

### TECHNICAL FIELD

The present invention relates a novel crystal of a glucokinase protein (hereinafter, referred to as GK protein), a drug design method using a three dimensional structure coordinate obtained by using this crystal, and the like.

### BACKGROUND ART

Glucokinase (ATP: D-hexose 6-phosphotransferaze, EC2. 7. 1. 1) is one (hexokinase IV) of four kinds of hexokinase isozymes of mammals. These isozymes catalyze the same reaction, however, show a difference in Km value for glucose. Namely, the Km values of hexokinases I, II, III are from 10⁻⁵ to 10⁻⁴ M, while the Km value for glucose of hexokinase IV also called glucokinase is by far larger, about 10⁻² M. Hexokinase is an enzyme correlated with the initial stage of the glycolysis system, and catalyzes a reaction from glucose into glucose-6-phosphate.

Manifestation of glucokinase is localized mainly in liver and pancreatic β cells, and by controlling the rate determining stage of glucose metabolism in these cells, glucokinase plays an important role in metabolism of the whole body. Glucokinases of liver and pancreatic β cells differ in a 15-amino acid sequence at the N-terminal due to difference in splicing, however, have the same enzymological nature.

For about 10 years, there is suggested a hypothesis that glucokinase works as a glucose sensor in pancreatic β cells and liver (Garfinkel D, et al: Am J Physiol 247 (3Pt2): R527-537, 1984). From recent results of glucokinase gene-manipulated mice, it has been clarified that glucokinase actually plays an important role in glucose homeostasis in the whole body.

A mouse having a glucokinase gene broken dies soon after birth by diabetes mellitus. (Grupe A, et al: Cell 83:69-78. 1995). On the other hand, a mouse having glucokinase expressed excessively shows lower blood glucose level (Ferre T, et al: Proc Natl Acad Sci USA 93: 7225-7230. 1996). When a glucokinase activity increases by increase in the concentration of glucose, the reaction of a pancreatic β cell and the reaction of a liver cell are different, however, both of them act to lower blood glucose level. By this, a pancreatic β cell secretes a larger amount of insulin, and liver incorporates glucose and store this as glycogen, and glucose release lowers simultaneously.

Variation of glucokinase enzymatic activity thus performs an important role in glucose homeostasis of mammals via liver and pancreatic β cells. In a case generating diabetes mellitus in younger ages called MODY2 (maturity-onset diabetes of the young), mutation of a glucokinase gene was found, and decrease in glucokinase activity causes increase in blood glucose value (Vionnet N, et al: Nature 356: 721-722, 1992). On the other hand, a family having mutation increasing a glucokinase activity was also found, and persons in such a family show hypoglycemia (Glaser B, et al: N Engl J Med 338: 226-230, 1998).

As described above, glucokinase acts as a glucose sensor also in humans and performs an important role in glucose homeostasis. On the other hand, since glucokinases of a lot of patients suffering from type II diabetes mellitus have no variation, blood glucose regulation utilizing a glucokinase sensor system is believed possible. A glucokinase activating substance is believed to be useful as a remedy for patients suffering from type II diabetes mellitus since this substance can be expected to have an action to promote insulation secretion of a pancreatic β cell and an action of accentuating incorporation of glucose of liver and suppressing release of glucose.

Recently, it has been clarified that pancreatic β cell type glucokinases are expressed locally in rat brains, among others, particularly ventromedial hypothalamus (VMH) which is a feeding center. About 20% neurocytes of VMH are called glucose response neuron, and conventionally believed to perform an important role in weight control. When glucose is dosed into a rat brain, feeding amount decreases, while when glucose metabolism is suppressed by administration of glucosamine which is a glucose analog into a brain, overeating occurs. It is known from electrophysiological experiments that a glucose responsive neuron is activated in response to physiological glucose concentration change (from 5 to 20 mM), while, when glucose metabolism is suppressed by glucosamine and the like, activity suppression is recognized. In a glucose concentration sensing system of VMH, a mechanism via glucokinase is hypothesized which is the same as that for insulation secretion of pancreatic β cells. Therefore, in addition to liver and pancreatic β cells, substances effecting glucokinase activation of VHM not only have an effect of correcting blood glucose value but also have a possibility of correcting also obesity which is a problem in may patients suffering from type II diabetes mellitus.

On the other hand, DIABETES, vol. 48, 1698-1705, September 1999 describes that the steric structure of glucokinase is predicted from hexokinase I, however, actually, it is nor crystallized and not practical.

From the above-described facts, to clarify the three dimensional steric structure of glucokinase and to enable efficient finding of a compound acting mutually with glucokinase are believed to make large progress in development of, for example, therapeutic agents or preventive preparations of diabetes mellitus; therapeutic agents or preventive preparations of chronic complications of diabetes mellitus such as retinopathy, nephropathy, neuropathy, ischemic heart disease, arterial sclerosis and the like; therapeutic agents or preventive preparations of obesity.

Currently, CARDD (Computer Aided Rational Drug Design) utilizing computers is frequently used at practical level, for analysis of the activity center and forecasting of the reaction mechanism, of a protein.

In a drug finding system by CARDD, the structure of the active portion of a protein is predicted based on the three-dimensional structure analysis data of a target protein. Information regarding the candidate of a compound capable of binding with the structure of its active portion is obtained from the database of the compound. Thereafter, candidates of a compound capable of binding to a target protein are selected, in view of the active portion of a target protein and the three-dimensional structure and physical nature of the candidate compound. These processes are a so-called insilico-screening process.

Whether a compound selected in the insilico-screening process is connected with a target protein to change its activity or not is checked by an actual test (wet experiment). A compound actually changing the activity of a target protein becomes an effective ingredient of a drug. By this, a compound interacting with a target protein can be efficiently found without conducting an operation of confirming an interaction by allowing a large amount of compounds to act respectively on target proteins in a laboratory.

The insilico-screening is judged to be a means effective for development of medical products since it can significantly narrow down candidate compounds which bind to a target protein.

In a drug finding system by CARDD, three dimensional structure analysis data by X-ray structure analysis of a target protein constitute a piece of important information. For three dimensional structure analysis by X-ray structure analysis, a crystal of a target protein is necessary as an analysis sample. Therefore, a crystal of GK is necessary for progressing the development of drug finding correlated with GK based on a drug finding system by CARDD. However, it is difficult to crystallize GK as described above, and it does not impart information necessary for CARDD.

### DISCLOSURE OF INVENTION

The present invention has been accomplished in view of the above-mentioned problems of conventional technologies, and has objects to obtain a crystal of glucokinase and to design a compound binding to glucokinase based on information obtained from the crystal.

At least one of the above-mentioned objects is solved by the following inventions.

[1] A glucokinase protein, used for crystallization.

[2] The protein according to [1], consisting of an amino acid sequence as depicted in SEQ ID No. 5.

[3] A crystal of a protein, consisting of an amino acid sequence as depicted in SEQ ID No. 5 or an amino acid sequence which is substantially the same as the amino acid sequence.

[4] The crystal according to [3], wherein the above-mentioned protein is a glucokinase protein.

[5] The crystal according to [3], which is a crystal of a protein having an amino acid sequence as depicted in SEQ ID No. 5.

[6] The crystal according to [3], wherein the lattice constant satisfies the following formulae (1) to (4):
a=b=79.9±4 Å (1)
c=322.2±15 Å (2)
α=β=90° (3)
γ=120° (4)

[7] The crystal according to [6], wherein the space group is P6₅22.

[8] A crystal of a protein specified by three dimensional structure coordinate data described in Table 1.

[9] A crystal wherein, in three dimensional structure coordinate data obtained by changing at least one datum in three dimensional structure coordinate data described in Table 1, the average square deviation between an atom (Cα atom) of a main chain of an amino acid shown by three dimensional structure coordinate data described in Table 1 and a Cα atom shown by the above-mentioned changed three dimensional structure coordinate data corresponding to the Cα atom is 0.6 Å or less.

[10] The crystal according to any of [3] to [9], wherein the compound binding portion is constituted of at least one of amino acid residues of tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459, in an amino acid sequence as depicted in SEQ ID No. 5.

[11] A crystal comprising a complex of a protein consisting of an amino acid sequence as depicted in SEQ ID No. 5 or an amino acid sequence which is substantially the same as the amino acid sequence with a compound capable of binding to the protein.

[12] The crystal according to [11], wherein the above-mentioned compound is represented by the formula (I): 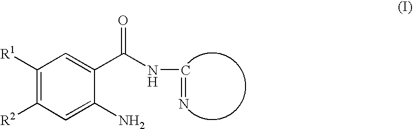
[wherein, R¹ represents a halogen atom, —S—(O)ₚ-A, —S—(O)_{q}—B or —O—B (wherein, p and q are the same or different and represent an integer of 0 to 2, A represents a straight chain C₁ to C₆ alkyl group optionally substituted and B represents a 5-membered cyclic or 6-membered cyclic aryl group or heteroaryl group optionally substituted), R² represents a hydrogen atom or halogen atom, and 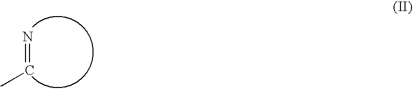
represents a mono-cyclic or di-cyclic heteroaryl group optionally substituted, having a nitrogen atom adjacent to a carbon atom connected to an amide group.].

[13] The crystal according to [12], wherein the above-mentioned compound is any of compounds of the formulae (IIIa) to (IIIc):

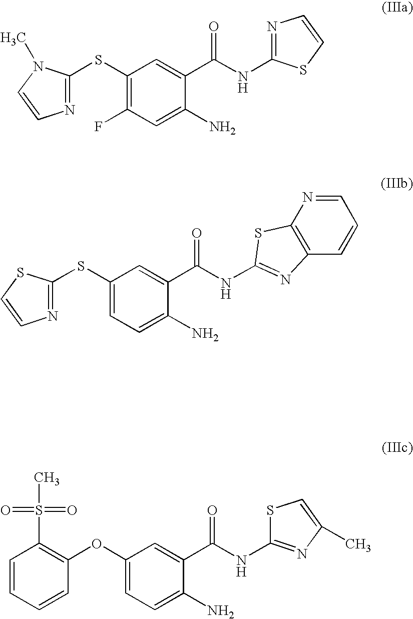

[14] The protein according to [1], consisting of an amino acid sequence as depicted in SEQ ID No. 8.

[15] A crystal of a protein, consisting of an amino acid sequence as depicted in SEQ ID No. 8 or an amino acid sequence which is substantially the same as the amino acid sequence.

[16] The crystal according to [15], wherein the above-mentioned protein is a glucokinase protein.

[17] The crystal according to [15], which is a crystal of a protein having an amino acid sequence as depicted in SEQ ID No. 8.

[18] The crystal according to [15], wherein the lattice constant satisfies the following formulae:
a=b=103.2±5 Å (5)
c=281.0±7 Å (6)
α=β=90° (7)
γ=120° (8)

[19] The crystal according to [18], wherein the space group is P6₅22.

[20] A crystal of a protein specified by three dimensional structure coordinate data described in Table 2.

[21] A crystal wherein, in three dimensional structure coordinate data obtained by changing at least one datum in three dimensional structure coordinate data described in Table 2, the average square deviation between an atom (Cα atom) of a main chain of an amino acid shown by three dimensional structure coordinate data described in Table 2 and a Cα atom shown by the above-mentioned changed three dimensional structure coordinate data corresponding to the Cα atom is 0.6 Å or less.

[22] A method of producing a crystal comprising a complex of a protein with a compound binding to the protein, comprising

a protein production step of producing a protein having an amino acid sequence obtained by deleting 1 to 50 amino acid residues from one or both of the N terminal and C terminal of a protein having an amino acid sequence as depicted in SEQ ID No. 2, and

a protein reaction step of reacting a compound binding to a protein obtained in the above-mentioned protein production step with a protein obtained in the above-mentioned protein production step.

[23] A method of producing a crystal of a protein, using a protein containing an amino acid sequence as depicted in SEQ ID No. 5 or an amino acid sequence which is substantially the same as the amino acid sequence, and having a glucokinase activity, and a compound capable of binding to the above-mentioned protein.

[24] The method of producing a crystal of a protein according to [23], wherein the above-mentioned compound capable of binding to the protein is a compound of the formula (I): 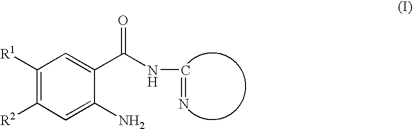
[wherein, R¹ represents a halogen atom, —S—(O)ₚ-A, —S—(O)_{q}—B or —O—B (wherein, p and q are the same or different and represent an integer of 0 to 2, A represents a straight chain C₁ to C₆ alkyl group optionally substituted and B represents a 5-membered cyclic or 6-membered cyclic aryl group or heteroaryl group optionally substituted), R² represents a hydrogen atom or halogen atom, and 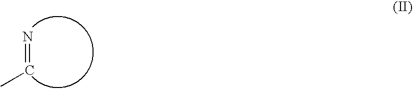
represents a mono-cyclic or di-cyclic heteroaryl group optionally substituted, having a nitrogen atom adjacent to a carbon atom connected to an amide group.].

[25] The method of producing a crystal according to [23] or [24], according to a co-crystallization method or soaking method.

[26] A drug design method of designing the structure of a compound capable of binding to a protein based on the steric structure information of the protein, wherein the steric structure information of the protein is information obtained by analyzing the crystal according to any of [3] to [13], [15] to [21].

[27] The drug design method according to [26], comprising

a binding portion guessing step of guessing a compound binding portion of the above-mentioned protein based on the above-mentioned steric structure information, and

a selection step of selecting a compound adaptable to a compound binding portion guessed in the above-mentioned binding portion guessing step from a compound library.

[28] The drug design method according to [26], comprising

a binding portion guessing step of guessing a compound binding portion of the above-mentioned protein based on the above-mentioned steric structure information, and

a compound structure fabricating step of fabricating the structure of a compound adaptable to a compound binding portion guessed in the above-mentioned binding portion guessing step.

[29] The drug design method according to [26], comprising

a binding portion guessing step of guessing a compound binding portion of the above-mentioned protein based on the above-mentioned steric structure information, and

a design step of designing visually the structure of a compound so that a compound binding portion guessed in the above-mentioned binding portion guessing step and a compound adaptable to the compound binding portion interact.

[30] The drug design method according to any of [26] to [29], wherein the above-mentioned compound binding portion is constituted of at least one of amino acid residues of tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459, in an amino acid sequence as depicted in SEQ ID No. 5.

[31] The drug design method according to any of [26] to [30], further comprising a step of measuring the physiological activity of a candidate compound guessed to be adaptable to the above-mentioned compound binding portion.

[32] The drug design method according to any of [26] to [30], further comprising a binding judgment step of contacting a candidate compound guessed to be adaptable to the above-mentioned compound binding portion with a protein containing an amino acid sequence as depicted in SEQ ID No. 5 or an amino acid sequence which is substantially the same as the amino acid sequence, and judging if the candidate compound binds to the protein or not.

[33] A method of producing a compound array, comprising combining groups of compounds selected by the drug design method according to any of [26] to [30], so as to be a compound array.

### BRIEF DESCRIPTION OF DRAWINGS

FIG. 1 is a ribbon view showing the three dimensional structure of glucokinase.

(FIG. 1a is a ribbon view showing the structure of glucokinase (Δ1-11)/glucose/compound 1 (compound of the formula IIIa). A right view is obtained by rotating a left view.)

(FIG. 1b is a ribbon view showing the structure of a glucokinase (Δ1-15) single entity. A right view is obtained by rotating a left view.)

FIG. 2 is a view showing a mode of binding of a compound 1 (compound of the formula IIIa) to the binding portion of glucokinase (Δ1-11).

FIG. 3 is a view showing the structure of the binding portion of glucokinase (Δ1-11).

### BEST MODE FOR CARRYING OUT THE INVENTION

In this specification, an amino acid, peptide and protein are represented by using abbreviations adopted in IUPAC-IUB Committee of Biochemical Nomenclature shown below. Unless otherwise stated, a sequence of an amino acid residue of a peptide and protein is represented so that the left end is the N-terminal and the right end is the C-terminal and the N-terminal is No. 1.

Embodiments of the invention will be described in detail below.

(Glucokinase Protein)

First, the present invention provides a glucokinase protein, used for crystallization. A glucokinase protein (GK protein) is involved in an extremely important saccharometabolism in organisms. Therefore, a compound binding to a GK protein (namely, activating agent or inhibitor) can be searched by clarifying the three dimensional structure of a GK protein and analyzing the active portion of a GK protein. That is, it is important to clarify the three dimensional structure of a GK protein.

As the means for clarifying the three dimensional structure of a protein, X-ray crystal structure analysis is well known. Namely, a protein is crystallized, the crystal is irradiated with monochromatized X-ray, and the three dimensional structure of this protein is analyzed based on the resulted X-ray diffraction image (Blundell, T. L. and Johnson, L. N., PROTEIN CRYSTALLOGRAPHY, 1-565, (1976) Academic Press, New York). It is necessary to first crystallize a GK protein, for analyzing the X-ray crystal structure of a GK protein.

Here, “GK protein” of the present invention means a protein containing a liver type glucokinase derived from human having an amino acid sequence as depicted in SEQ ID No. 2 and an amino acid sequence which is substantially the same as that of SEQ ID No. 2. Here, this protein containing substantially the same amino acid sequence is preferably that having a glucokinase activity. Accordingly, in this specification, the GK protein includes not only a liver type glucokinase derived from human but also a pancreas type glucokinase derived from human, and GK proteins derived from animals other than human such as mouse, rat, monkey and the like. In the present invention, a human liver type glucokinase is preferably used. In human-derived glucokinases, that of liver type and that of pancreas type differ in a 15-amino acid residue at the N-terminal. Here, “glucokinase activity” means an activity catalyzing a reaction from glucose into glucose-6-phosphate.

It is well known that crystallization of a protein is generally difficult, consequently, a GK protein could not be crystallized as it was. The present inventors have conducted various experiments through trial and error, and resultantly succeeded for the first time in crystallization of a GK protein by deleting 11 or 15 amino acids a the N-terminal side of a GK protein. It is believed that the deleted region projects from a spherical GK protein molecule in trying crystallization, as a result, constitutes steric hindrance between a GK protein molecule adjacent in the crystal, to prevent crystallization of a GK protein. Namely, in the present invention, a crystal of a GK protein was obtained by using a GK protein including deletion of 11 amino acid residues at the N-terminal (SEQ ID No. 5) or a GK protein including deletion of 15 amino acid residues at the N-terminal (SEQ ID No. 8), in a glucokinase of which amino acid sequence was known, however, of which crystallization had not been succeeded. However, the number of amino acids to be deleted is not limited providing steric hindrance is not formed between an adjacent crystal. Specifically, amino acid sequences obtained by deleting 1 to 50, preferably 3 to 30, more preferably 5 to 25, further preferably 8 to 18, particularly preferably 11 to 15 amino acid residues at the N-terminal in an amino acid sequence as depicted in SEQ ID No. 2, are used in the present invention. Further, amino acid sequences including deletion of 1 to 8, preferably 1 to 7, more preferably 2 to 6 amino acid residues at the C-terminal are used in the present invention.

(Crystal of Glucokinase Protein and Production Method thereof)

Next, in the present invention, a crystal comprising a protein containing an amino acid sequence as depicted in SEQ ID No. 5, SEQ ID No. 8 or an amino acid sequence which is substantially the same as this amino acid sequence is provided.

As described above, as a GK protein to be crystallized, a protein containing an amino acid sequence as depicted in SEQ ID No. 5 and/or SEQ ID No. 8 or an amino acid sequence which is substantially the same as this amino acid sequence, and the like are provided.

A protein containing an amino acid sequence as depicted in SEQ ID No. 5 and/or SEQ ID No. 8 or an amino acid sequence which is substantially the same as this amino acid sequence (hereinafter, abbreviated in some cases as “GK protein”, also including a protein having an amino acid sequence as depicted in SEQ ID No. 2 or an amino acid sequence which is substantially the same as this amino acid sequence) may be permissible providing crystallization thereof is possible, and its amino acid sequence is not particularly restricted. Here, a protein containing an amino acid sequence which is substantially the same as an amino acid sequence as depicted in SEQ ID No. 5 and/or SEQ ID No. 8 is not required to have a glucokinase activity, and may also be an inactive variant (for example, a variant obtained by inactivation through a variation at a binding portion of ATP) providing it has a crystal structure capable of providing information necessary for drug design. Here, the protein containing an amino acid sequence which is substantially the same as an amino acid sequence as depicted in SEQ ID No. 2 or 5 includes amino acid sequences having a homology of about 60% or more, preferably about 70% or more, further preferably about 80% or more, particularly preferably about 90% or more, most preferably about 95% or more with the amino acid sequence as depicted in SEQ ID No. 2 or 5. Examples of the protein containing an amino acid sequence which is substantially the same as an amino acid sequence as depicted in SEQ ID No. 2 or 5 include amino acid sequences obtained by substitution, deletion, addition and/or insertion of 1 to 10, preferably 1 to 5, further preferably 1 to 3, further preferably 1 to 2 amino acid residues in an amino acid sequence as depicted in SEQ ID No. 2 or 5.

Analysis of the three dimensional structure of a GK protein is conducted, for example, as described below. First, a protein is purified. Then, a series of processes such as crystallization, X-ray diffraction intensity data collection, determination of phases of diffraction spots, electron density calculation, molecule model making, precise structure formation and the like are conducted. As main equipments for conducting analysis of a protein structure, an incubator for crystallization, binocular microscope, X-ray diffractometer, three dimensional computer graphics apparatus and the like are used. Specifically, an experimental process for producing a crystal of a protein is divided into a stage of purifying a large amount of protein (preferably, several mg or more), a stage of widely searching conditions for obtaining a crystal, and a stage of obtaining a crystal of good quality suitable for X-ray analysis. These processes will be described specifically below.

In crystallization, a GK protein is purified at high purity. As the purification method, known methods can be utilized, and for example, column chromatography, salting out, centrifugal separation and the like are used.

The purified GK protein is crystallized and used as a sample for analysis of an X-ray crystal structure. Crystallization is conducted based on known methods such as a vapor diffusion method, dialysis method and the like. In obtaining a crystal of a protein, it is necessary to investigate a lot of factors such as the purity and concentration of a protein, temperature, pH, the concentration of a precipitating agent used, and the like. Investigation of crystallization conditions can be conducted in a wider range using a commercially available screening reagent, and it is preferable to conduct searching using 1 to 2 μL of a protein solution having a concentration of 1 to 2% for one condition. When a fine crystal is thus obtained, it is preferable to further refine the condition.

For obtaining a crystal of a GK protein, very many conditions should be searched. Therefore, also for investigating crystallization conditions, it is preferable to fabricate a system of expressing a large amount of protein. In general, a significant proportion of proteins which are crystallized are in mono-dispersion in a solution condition, and those in poly-dispersion are not crystallized in many cases. Then, it may also be permissible that the N-terminals of GK proteins are sequentially cut, a mono-dispersibility of a solution of the resulted protein is judged using a light scattering apparatus, and whether a sample is suitable for crystallization or not is investigated.

Next, X-ray diffraction intensity measurement is conducted using a crystal of the resulted GK protein. Recently, there is also utilized a method in which a crystal is scooped by a ring of narrow string and the like and cooled quickly to a liquid nitrogen temperature, and measurement is conducted as it is at lower temperatures. Measurement of the intensity of diffracted X-ray is usually conducted by a two dimensional detector such as an imaging plate and the like. A lot of diffracted beams generated by rotating a crystal while irradiating with X-ray are recorded on an imaging plate, and the recorded diffraction intensity is read by irradiating with laser.

Next, it is preferable to prepare a heavy atom isomorphic substituted body by a heavy atom soaking method or co-crystallization method. Using this, the phase of a protein crystal can be determined by a multiple isomorphic substitution method (MIR method). Instead of introduction of a heavy atom, there can be also utilized a multi-wavelength abnormal diffusion method (MAD method) determining a phase based on diffraction intensity data by X-ray of complicated wavelength. When a molecule having an analogous structure is already analyzed, its molecule structure can be applied into a crystal to give the initial structure, and there is also known a molecule substitution method (MR method) of drawing a Fourier synthesis diagram based on the resulted initial structure, and analyzing the structure of remaining portions to determine the whole structure.

When a phase is determined by the above-mentioned method, electron density is calculated based on this. This precision depends on the number of reflection (resolution ability) and precision of reflection used. The resolution ability is represented by minimum plane interval of reflection used. Base on this electron density diagram, a molecule model is fabricated. When a molecule model is fabricated, an atomic coordinate is obtained, and by this, a calculation value of structural factors is obtained, and preciseness of atomic parameter is increased by a minimum square method of approximating this value to an observed value. Thus, structure information which is as reasonable as possible is obtained.

The present invention has succeeded in preparing a crystal of a GK protein as depicted in SEQ ID No. 5 (see, examples below). Thus obtained crystal of a GK protein had a lattice constant satisfying the following formulae (1) to (4):
a=b=79.9±4 Å (1)
c=322.2±15 Å (2)
α=β=90° (3)
γ=120° (4)
This crystal was analyzed to have a space group of P6₅22. Here, the above-mentioned a=b is preferably 79.9±3 Å, more preferably 79.9±2 Å, further preferably 79.9±1 Å. The above-mentioned c is preferably 322.2±10 Å, more preferably 322.2±8 Å, further preferably 322.2±5 Å.

The three dimensional structure coordinate of thus obtained GK protein crystal is shown in Table 1.

TABLE 1

| ATOM | 1 | CB | THR | 14 | 25.972 | −34.025 | 76.567 | 1.00 | 51.12 |
|---|---|---|---|---|---|---|---|---|---|
| ATOM | 2 | OG1 | THR | 14 | 27.398 | −34.012 | 76.715 | 1.00 | 51.49 |
| ATOM | 3 | CG2 | THR | 14 | 25.626 | −34.173 | 75.095 | 1.00 | 49.96 |
| ATOM | 4 | C | THR | 14 | 24.138 | −32.317 | 76.374 | 1.00 | 50.95 |
| ATOM | 5 | O | THR | 14 | 24.246 | −31.685 | 75.330 | 1.00 | 52.42 |
| ATOM | 6 | N | THR | 14 | 25.108 | −32.861 | 78.611 | 1.00 | 51.41 |
| ATOM | 7 | CA | THR | 14 | 25.384 | −32.717 | 77.154 | 1.00 | 50.49 |
| ATOM | 8 | N | LEU | 15 | 22.957 | −32.673 | 76.871 | 1.00 | 49.75 |
| ATOM | 9 | CA | LEU | 15 | 21.733 | −32.307 | 76.167 | 1.00 | 49.25 |
| ATOM | 10 | CB | LEU | 15 | 20.496 | −32.824 | 76.904 | 1.00 | 52.56 |
| ATOM | 11 | CG | LEU | 15 | 20.439 | −34.307 | 77.291 | 1.00 | 55.08 |
| ATOM | 12 | CD1 | LEU | 15 | 21.186 | −34.524 | 78.610 | 1.00 | 53.67 |
| ATOM | 13 | CD2 | LEU | 15 | 18.980 | −34.742 | 77.438 | 1.00 | 54.84 |
| ATOM | 14 | C | LEU | 15 | 21.676 | −30.781 | 76.078 | 1.00 | 48.68 |
| ATOM | 15 | O | LEU | 15 | 21.397 | −30.208 | 75.023 | 1.00 | 47.52 |
| ATOM | 16 | N | VAL | 16 | 21.955 | −30.128 | 77.201 | 1.00 | 47.07 |
| ATOM | 17 | CA | VAL | 16 | 21.950 | −28.677 | 77.265 | 1.00 | 44.96 |
| ATOM | 18 | CB | VAL | 16 | 21.988 | −28.188 | 78.733 | 1.00 | 46.09 |
| ATOM | 19 | CG1 | VAL | 16 | 22.239 | −26.684 | 78.784 | 1.00 | 44.09 |
| ATOM | 20 | CG2 | VAL | 16 | 20.670 | −28.523 | 79.418 | 1.00 | 45.38 |
| ATOM | 21 | C | VAL | 16 | 23.142 | −28.097 | 76.512 | 1.00 | 43.58 |
| ATOM | 22 | O | VAL | 16 | 23.004 | −27.110 | 75.790 | 1.00 | 41.54 |
| ATOM | 23 | N | GLU | 17 | 24.310 | −28.712 | 76.672 | 1.00 | 43.48 |
| ATOM | 24 | CA | GLU | 17 | 25.507 | −28.223 | 75.998 | 1.00 | 45.62 |
| ATOM | 25 | CB | GLU | 17 | 26.759 | −28.931 | 76.532 | 1.00 | 46.30 |
| ATOM | 26 | CG | GLU | 17 | 27.140 | −28.571 | 77.984 | 1.00 | 49.19 |
| ATOM | 27 | CD | GLU | 17 | 27.467 | −27.087 | 78.191 | 1.00 | 50.74 |
| ATOM | 28 | OE1 | GLU | 17 | 28.238 | −26.520 | 77.386 | 1.00 | 50.39 |
| ATOM | 29 | OE2 | GLU | 17 | 26.966 | −26.488 | 79.170 | 1.00 | 50.85 |
| ATOM | 30 | C | GLU | 17 | 25.417 | −28.378 | 74.479 | 1.00 | 45.93 |
| ATOM | 31 | O | GLU | 17 | 26.097 | −27.666 | 73.735 | 1.00 | 44.10 |
| ATOM | 32 | N | GLN | 18 | 24.577 | −29.303 | 74.020 | 1.00 | 45.41 |
| ATOM | 33 | CA | GLN | 18 | 24.400 | −29.513 | 72.588 | 1.00 | 46.37 |
| ATOM | 34 | CB | GLN | 18 | 23.643 | −30.818 | 72.307 | 1.00 | 49.99 |
| ATOM | 35 | CG | GLN | 18 | 24.488 | −32.086 | 72.423 | 1.00 | 55.59 |
| ATOM | 36 | CD | GLN | 18 | 23.701 | −33.352 | 72.088 | 1.00 | 58.40 |
| ATOM | 37 | OE1 | GLN | 18 | 23.158 | −33.489 | 70.988 | 1.00 | 60.78 |
| ATOM | 38 | NE2 | GLN | 18 | 23.638 | −34.280 | 73.037 | 1.00 | 56.40 |
| ATOM | 39 | C | GLN | 18 | 23.617 | −28.338 | 72.014 | 1.00 | 44.35 |
| ATOM | 40 | O | GLN | 18 | 23.849 | −27.912 | 70.885 | 1.00 | 43.20 |
| ATOM | 41 | N | ILE | 19 | 22.677 | −27.821 | 72.791 | 1.00 | 41.97 |
| ATOM | 42 | CA | ILE | 19 | 21.895 | −26.689 | 72.327 | 1.00 | 40.37 |
| ATOM | 43 | CB | ILE | 19 | 20.631 | −26.500 | 73.193 | 1.00 | 39.71 |
| ATOM | 44 | CG2 | ILE | 19 | 19.976 | −25.166 | 72.894 | 1.00 | 39.42 |
| ATOM | 45 | CG1 | ILE | 19 | 19.653 | −27.653 | 72.915 | 1.00 | 40.83 |
| ATOM | 46 | CD1 | ILE | 19 | 18.356 | −27.599 | 73.719 | 1.00 | 38.38 |
| ATOM | 47 | C | ILE | 19 | 22.764 | −25.431 | 72.344 | 1.00 | 39.01 |
| ATOM | 48 | O | ILE | 19 | 22.746 | −24.644 | 71.394 | 1.00 | 40.12 |
| ATOM | 49 | N | LEU | 20 | 23.550 | −25.267 | 73.404 | 1.00 | 35.38 |
| ATOM | 50 | CA | LEU | 20 | 24.423 | −24.109 | 73.537 | 1.00 | 34.35 |
| ATOM | 51 | CB | LEU | 20 | 25.026 | −24.050 | 74.944 | 1.00 | 32.09 |
| ATOM | 52 | CG | LEU | 20 | 24.050 | −23.887 | 76.106 | 1.00 | 30.92 |
| ATOM | 53 | CD1 | LEU | 20 | 24.813 | −23.722 | 77.420 | 1.00 | 27.61 |
| ATOM | 54 | CD2 | LEU | 20 | 23.171 | −22.689 | 75.843 | 1.00 | 29.31 |
| ATOM | 55 | C | LEU | 20 | 25.555 | −24.135 | 72.518 | 1.00 | 34.62 |
| ATOM | 56 | O | LEU | 20 | 26.066 | −23.087 | 72.112 | 1.00 | 34.19 |
| ATOM | 57 | N | ALA | 21 | 25.946 | −25.336 | 72.116 | 1.00 | 33.16 |
| ATOM | 58 | CA | ALA | 21 | 27.030 | −25.509 | 71.163 | 1.00 | 34.30 |
| ATOM | 59 | CB | ALA | 21 | 27.344 | −26.992 | 70.993 | 1.00 | 34.49 |
| ATOM | 60 | C | ALA | 21 | 26.696 | −24.886 | 69.814 | 1.00 | 35.20 |
| ATOM | 61 | O | ALA | 21 | 27.587 | −24.619 | 69.007 | 1.00 | 35.57 |
| ATOM | 62 | N | GLU | 22 | 25.412 | −24.652 | 69.578 | 1.00 | 36.75 |
| ATOM | 63 | CA | GLU | 22 | 24.961 | −24.053 | 68.329 | 1.00 | 37.80 |
| ATOM | 64 | CB | GLU | 22 | 23.435 | −24.102 | 68.256 | 1.00 | 41.47 |
| ATOM | 65 | CG | GLU | 22 | 22.878 | −23.851 | 66.867 | 1.00 | 47.91 |
| ATOM | 66 | CD | GLU | 22 | 21.384 | −24.128 | 66.767 | 1.00 | 49.95 |
| ATOM | 67 | OE1 | GLU | 22 | 20.857 | −24.163 | 65.630 | 1.00 | 50.84 |
| ATOM | 68 | OE2 | GLU | 22 | 20.741 | −24.307 | 67.822 | 1.00 | 50.26 |
| ATOM | 69 | C | GLU | 22 | 25.444 | −22.605 | 68.177 | 1.00 | 37.38 |
| ATOM | 70 | O | GLU | 22 | 25.380 | −22.039 | 67.088 | 1.00 | 38.34 |
| ATOM | 71 | N | PHE | 23 | 25.928 | −22.012 | 69.268 | 1.00 | 35.41 |
| ATOM | 72 | CA | PHE | 23 | 26.426 | −20.636 | 69.249 | 1.00 | 33.38 |
| ATOM | 73 | CB | PHE | 23 | 26.224 | −19.962 | 70.614 | 1.00 | 31.59 |
| ATOM | 74 | CG | PHE | 23 | 24.826 | −19.470 | 70.843 | 1.00 | 29.81 |
| ATOM | 75 | CD1 | PHE | 23 | 23.836 | −20.328 | 71.310 | 1.00 | 26.48 |
| ATOM | 76 | CD2 | PHE | 23 | 24.489 | −18.151 | 70.555 | 1.00 | 28.79 |
| ATOM | 77 | CE1 | PHE | 23 | 22.520 | −19.882 | 71.487 | 1.00 | 29.30 |
| ATOM | 78 | CE2 | PHE | 23 | 23.177 | −17.691 | 70.727 | 1.00 | 31.65 |
| ATOM | 79 | CZ | PHE | 23 | 22.189 | −18.563 | 71.195 | 1.00 | 28.91 |
| ATOM | 80 | C | PHE | 23 | 27.899 | −20.542 | 68.877 | 1.00 | 33.33 |
| ATOM | 81 | O | PHE | 23 | 28.396 | −19.467 | 68.549 | 1.00 | 34.12 |
| ATOM | 82 | N | GLN | 24 | 28.596 | −21.670 | 68.940 | 1.00 | 32.75 |
| ATOM | 83 | CA | GLN | 24 | 30.016 | −21.716 | 68.620 | 1.00 | 32.56 |
| ATOM | 84 | CB | GLN | 24 | 30.543 | −23.147 | 68.778 | 1.00 | 35.53 |
| ATOM | 85 | CG | GLN | 24 | 30.817 | −23.603 | 70.210 | 1.00 | 37.84 |
| ATOM | 86 | CD | GLN | 24 | 31.214 | −25.074 | 70.266 | 1.00 | 42.36 |
| ATOM | 87 | OE1 | GLN | 24 | 31.802 | −25.601 | 69.320 | 1.00 | 43.06 |
| ATOM | 88 | NE2 | GLN | 24 | 30.902 | −25.739 | 71.375 | 1.00 | 40.61 |
| ATOM | 89 | C | GLN | 24 | 30.335 | −21.233 | 67.208 | 1.00 | 31.93 |
| ATOM | 90 | O | GLN | 24 | 29.508 | −21.320 | 66.299 | 1.00 | 30.32 |
| ATOM | 91 | N | LEU | 25 | 31.548 | −20.717 | 67.043 | 1.00 | 31.64 |
| ATOM | 92 | CA | LEU | 25 | 32.029 | −20.257 | 65.751 | 1.00 | 31.85 |
| ATOM | 93 | CB | LEU | 25 | 31.876 | −18.742 | 65.615 | 1.00 | 31.24 |
| ATOM | 94 | CG | LEU | 25 | 30.441 | −18.211 | 65.563 | 1.00 | 29.93 |
| ATOM | 95 | CD1 | LEU | 25 | 30.436 | −16.690 | 65.710 | 1.00 | 28.63 |
| ATOM | 96 | CD2 | LEU | 25 | 29.801 | −18.640 | 64.262 | 1.00 | 27.61 |
| ATOM | 97 | C | LEU | 25 | 33.502 | −20.635 | 65.667 | 1.00 | 33.30 |
| ATOM | 98 | O | LEU | 25 | 34.298 | −20.218 | 66.502 | 1.00 | 33.97 |
| ATOM | 99 | N | GLN | 26 | 33.856 | −21.450 | 64.679 | 1.00 | 34.57 |
| ATOM | 100 | CA | GLN | 26 | 35.244 | −21.860 | 64.496 | 1.00 | 36.87 |
| ATOM | 101 | CB | GLN | 26 | 35.330 | −23.053 | 63.540 | 1.00 | 40.20 |
| ATOM | 102 | CG | GLN | 26 | 35.105 | −24.414 | 64.182 | 1.00 | 46.34 |
| ATOM | 103 | CD | GLN | 26 | 33.863 | −24.462 | 65.041 | 1.00 | 48.48 |
| ATOM | 104 | OE1 | GLN | 26 | 33.918 | −24.229 | 66.253 | 1.00 | 49.27 |
| ATOM | 105 | NE2 | GLN | 26 | 32.725 | −24.757 | 64.417 | 1.00 | 51.72 |
| ATOM | 106 | C | GLN | 26 | 36.024 | −20.688 | 63.910 | 1.00 | 36.49 |
| ATOM | 107 | O | GLN | 26 | 35.430 | −19.735 | 63.403 | 1.00 | 35.76 |
| ATOM | 108 | N | GLU | 27 | 37.347 | −20.761 | 63.981 | 1.00 | 35.17 |
| ATOM | 109 | CA | GLU | 27 | 38.181 | −19.705 | 63.441 | 1.00 | 37.77 |
| ATOM | 110 | CB | GLU | 27 | 39.658 | −20.047 | 63.627 | 1.00 | 40.11 |
| ATOM | 111 | CG | GLU | 27 | 40.596 | −19.156 | 62.831 | 1.00 | 47.14 |
| ATOM | 112 | CD | GLU | 27 | 41.754 | −18.639 | 63.662 | 1.00 | 52.56 |
| ATOM | 113 | OE1 | GLU | 27 | 41.507 | −17.808 | 64.567 | 1.00 | 54.72 |
| ATOM | 114 | OE2 | GLU | 27 | 42.906 | −19.067 | 63.415 | 1.00 | 54.43 |
| ATOM | 115 | C | GLU | 27 | 37.878 | −19.511 | 61.961 | 1.00 | 37.80 |
| ATOM | 116 | O | GLU | 27 | 37.915 | −18.392 | 61.446 | 1.00 | 37.09 |
| ATOM | 117 | N | GLU | 28 | 37.557 | −20.605 | 61.282 | 1.00 | 36.94 |
| ATOM | 118 | CA | GLU | 28 | 37.261 | −20.535 | 59.862 | 1.00 | 36.18 |
| ATOM | 119 | CB | GLU | 28 | 37.175 | −21.939 | 59.267 | 1.00 | 37.83 |
| ATOM | 120 | CG | GLU | 28 | 37.826 | −22.039 | 57.902 | 1.00 | 41.72 |
| ATOM | 121 | CD | GLU | 28 | 39.154 | −21.287 | 57.843 | 1.00 | 44.57 |
| ATOM | 122 | OE1 | GLU | 28 | 40.033 | −21.531 | 58.706 | 1.00 | 46.91 |
| ATOM | 123 | OE2 | GLU | 28 | 39.313 | −20.446 | 56.933 | 1.00 | 44.10 |
| ATOM | 124 | C | GLU | 28 | 35.973 | −19.779 | 59.588 | 1.00 | 34.66 |
| ATOM | 125 | O | GLU | 28 | 35.860 | −19.089 | 58.575 | 1.00 | 33.91 |
| ATOM | 126 | N | ASP | 29 | 34.994 | −19.926 | 60.472 | 1.00 | 32.44 |
| ATOM | 127 | CA | ASP | 29 | 33.738 | −19.219 | 60.301 | 1.00 | 32.41 |
| ATOM | 128 | CB | ASP | 29 | 32.713 | −19.625 | 61.370 | 1.00 | 34.13 |
| ATOM | 129 | CG | ASP | 29 | 32.302 | −21.091 | 61.285 | 1.00 | 34.13 |
| ATOM | 130 | OD1 | ASP | 29 | 32.012 | −21.580 | 60.173 | 1.00 | 34.03 |
| ATOM | 131 | OD2 | ASP | 29 | 32.246 | −21.749 | 62.347 | 1.00 | 35.16 |
| ATOM | 132 | C | ASP | 29 | 34.054 | −17.728 | 60.456 | 1.00 | 31.21 |
| ATOM | 133 | O | ASP | 29 | 33.542 | −16.895 | 59.717 | 1.00 | 29.93 |
| ATOM | 134 | N | LEU | 30 | 34.912 | −17.403 | 61.419 | 1.00 | 29.60 |
| ATOM | 135 | CA | LEU | 30 | 35.274 | −16.016 | 61.674 | 1.00 | 28.38 |
| ATOM | 136 | CB | LEU | 30 | 36.101 | −15.901 | 62.964 | 1.00 | 23.67 |
| ATOM | 137 | CG | LEU | 30 | 35.435 | −16.298 | 64.289 | 1.00 | 23.54 |
| ATOM | 138 | CD1 | LEU | 30 | 36.314 | −15.823 | 65.433 | 1.00 | 22.55 |
| ATOM | 139 | CD2 | LEU | 30 | 34.038 | −15.674 | 64.418 | 1.00 | 24.55 |
| ATOM | 140 | C | LEU | 30 | 36.032 | −15.390 | 60.499 | 1.00 | 29.80 |
| ATOM | 141 | O | LEU | 30 | 35.775 | −14.242 | 60.139 | 1.00 | 29.56 |
| ATOM | 142 | N | LYS | 31 | 36.963 | −16.131 | 59.906 | 1.00 | 29.13 |
| ATOM | 143 | CA | LYS | 31 | 37.704 | −15.609 | 58.770 | 1.00 | 30.46 |
| ATOM | 144 | CB | LYS | 31 | 38.823 | −16.574 | 58.365 | 1.00 | 32.24 |
| ATOM | 145 | CG | LYS | 31 | 39.970 | −16.653 | 59.374 | 1.00 | 36.80 |
| ATOM | 146 | CD | LYS | 31 | 41.091 | −17.577 | 58.885 | 1.00 | 40.49 |
| ATOM | 147 | CE | LYS | 31 | 42.291 | −17.534 | 59.829 | 1.00 | 44.52 |
| ATOM | 148 | NZ | LYS | 31 | 43.443 | −18.369 | 59.363 | 1.00 | 47.22 |
| ATOM | 149 | C | LYS | 31 | 36.746 | −15.391 | 57.599 | 1.00 | 31.28 |
| ATOM | 150 | O | LYS | 31 | 36.918 | −14.464 | 56.816 | 1.00 | 32.79 |
| ATOM | 151 | N | LYS | 32 | 35.730 | −16.243 | 57.486 | 1.00 | 30.96 |
| ATOM | 152 | CA | LYS | 32 | 34.758 | −16.116 | 56.406 | 1.00 | 32.66 |
| ATOM | 153 | CB | LYS | 32 | 33.868 | −17.364 | 56.324 | 1.00 | 32.27 |
| ATOM | 154 | CG | LYS | 32 | 32.921 | −17.362 | 55.135 | 1.00 | 34.72 |
| ATOM | 155 | CD | LYS | 32 | 32.203 | −18.701 | 54.965 | 1.00 | 39.55 |
| ATOM | 156 | CE | LYS | 32 | 31.272 | −18.678 | 53.745 | 1.00 | 42.65 |
| ATOM | 157 | NZ | LYS | 32 | 30.699 | −20.026 | 53.417 | 1.00 | 42.72 |
| ATOM | 158 | C | LYS | 32 | 33.890 | −14.868 | 56.609 | 1.00 | 32.63 |
| ATOM | 159 | O | LYS | 32 | 33.607 | −14.140 | 55.652 | 1.00 | 32.25 |
| ATOM | 160 | N | VAL | 33 | 33.463 | −14.629 | 57.847 | 1.00 | 30.17 |
| ATOM | 161 | CA | VAL | 33 | 32.654 | −13.451 | 58.149 | 1.00 | 29.03 |
| ATOM | 162 | CB | VAL | 33 | 32.154 | −13.460 | 59.626 | 1.00 | 30.49 |
| ATOM | 163 | CG1 | VAL | 33 | 31.519 | −12.123 | 59.985 | 1.00 | 31.03 |
| ATOM | 164 | CG2 | VAL | 33 | 31.130 | −14.562 | 59.815 | 1.00 | 32.03 |
| ATOM | 165 | C | VAL | 33 | 33.538 | −12.226 | 57.908 | 1.00 | 26.62 |
| ATOM | 166 | O | VAL | 33 | 33.091 | −11.237 | 57.338 | 1.00 | 22.25 |
| ATOM | 167 | N | MET | 34 | 34.802 | −12.321 | 58.317 | 1.00 | 25.50 |
| ATOM | 168 | CA | MET | 34 | 35.750 | −11.226 | 58.142 | 1.00 | 27.22 |
| ATOM | 169 | CB | MET | 34 | 37.108 | −11.583 | 58.748 | 1.00 | 24.41 |
| ATOM | 170 | CG | MET | 34 | 38.150 | −10.512 | 58.537 | 1.00 | 26.32 |
| ATOM | 171 | SD | MET | 34 | 39.793 | −11.040 | 59.074 | 1.00 | 32.95 |
| ATOM | 172 | CE | MET | 34 | 40.162 | −12.313 | 57.821 | 1.00 | 30.64 |
| ATOM | 173 | C | MET | 34 | 35.927 | −10.879 | 56.665 | 1.00 | 29.30 |
| ATOM | 174 | O | MET | 34 | 35.850 | −9.717 | 56.286 | 1.00 | 29.01 |
| ATOM | 175 | N | ARG | 35 | 36.164 | −11.883 | 55.827 | 1.00 | 30.96 |
| ATOM | 176 | CA | ARG | 35 | 36.340 | −11.621 | 54.403 | 1.00 | 32.99 |
| ATOM | 177 | CB | ARG | 35 | 36.664 | −12.913 | 53.641 | 1.00 | 34.85 |
| ATOM | 178 | CG | ARG | 35 | 37.948 | −13.585 | 54.081 | 1.00 | 38.82 |
| ATOM | 179 | CD | ARG | 35 | 38.377 | −14.682 | 53.126 | 1.00 | 43.22 |
| ATOM | 180 | NE | ARG | 35 | 38.963 | −15.791 | 53.869 | 1.00 | 47.35 |
| ATOM | 181 | CZ | ARG | 35 | 38.260 | −16.801 | 54.366 | 1.00 | 47.12 |
| ATOM | 182 | NH1 | ARG | 35 | 36.946 | −16.850 | 54.186 | 1.00 | 48.27 |
| ATOM | 183 | NH2 | ARG | 35 | 38.868 | −17.746 | 55.064 | 1.00 | 50.91 |
| ATOM | 184 | C | ARG | 35 | 35.090 | −10.997 | 53.797 | 1.00 | 33.31 |
| ATOM | 185 | O | ARG | 35 | 35.178 | −10.089 | 52.966 | 1.00 | 33.49 |
| ATOM | 186 | N | ARG | 36 | 33.926 | −11.493 | 54.206 | 1.00 | 32.00 |
| ATOM | 187 | CA | ARG | 36 | 32.673 | −10.982 | 53.675 | 1.00 | 31.76 |
| ATOM | 188 | CB | ARG | 36 | 31.511 | −11.857 | 54.158 | 1.00 | 29.95 |
| ATOM | 189 | CG | ARG | 36 | 30.191 | −11.607 | 53.441 | 1.00 | 31.90 |
| ATOM | 190 | CD | ARG | 36 | 30.386 | −11.434 | 51.929 | 1.00 | 33.67 |
| ATOM | 191 | NE | ARG | 36 | 29.114 | −11.263 | 51.230 | 1.00 | 38.02 |
| ATOM | 192 | CZ | ARG | 36 | 28.229 | −12.238 | 51.018 | 1.00 | 40.67 |
| ATOM | 193 | NH1 | ARG | 36 | 28.477 | −13.471 | 51.447 | 1.00 | 40.50 |
| ATOM | 194 | NH2 | ARG | 36 | 27.087 | −11.979 | 50.382 | 1.00 | 41.02 |
| ATOM | 195 | C | ARG | 36 | 32.459 | −9.510 | 54.060 | 1.00 | 31.54 |
| ATOM | 196 | O | ARG | 36 | 31.959 | −8.718 | 53.260 | 1.00 | 30.75 |
| ATOM | 197 | N | MET | 37 | 32.856 | −9.147 | 55.276 | 1.00 | 30.98 |
| ATOM | 198 | CA | MET | 37 | 32.720 | −7.774 | 55.742 | 1.00 | 30.21 |
| ATOM | 199 | CB | MET | 37 | 33.134 | −7.663 | 57.208 | 1.00 | 27.60 |
| ATOM | 200 | CG | MET | 37 | 33.102 | −6.240 | 57.761 | 1.00 | 27.98 |
| ATOM | 201 | SD | MET | 37 | 31.418 | −5.613 | 57.981 | 1.00 | 30.18 |
| ATOM | 202 | CE | MET | 37 | 31.115 | −6.153 | 59.683 | 1.00 | 28.30 |
| ATOM | 203 | C | MET | 37 | 33.598 | −6.852 | 54.892 | 1.00 | 30.32 |
| ATOM | 204 | O | MET | 37 | 33.162 | −5.782 | 54.479 | 1.00 | 31.66 |
| ATOM | 205 | N | GLN | 38 | 34.835 | −7.272 | 54.642 | 1.00 | 30.60 |
| ATOM | 206 | CA | GLN | 38 | 35.774 | −6.500 | 53.829 | 1.00 | 31.68 |
| ATOM | 207 | CB | GLN | 38 | 37.126 | −7.206 | 53.750 | 1.00 | 32.18 |
| ATOM | 208 | CG | GLN | 38 | 38.051 | −6.918 | 54.898 | 1.00 | 36.36 |
| ATOM | 209 | CD | GLN | 38 | 39.318 | −7.743 | 54.831 | 1.00 | 37.65 |
| ATOM | 210 | OE1 | GLN | 38 | 39.352 | −8.890 | 55.275 | 1.00 | 41.25 |
| ATOM | 211 | NE2 | GLN | 38 | 40.362 | −7.170 | 54.258 | 1.00 | 39.99 |
| ATOM | 212 | C | GLN | 38 | 35.241 | −6.337 | 52.419 | 1.00 | 32.20 |
| ATOM | 213 | O | GLN | 38 | 35.471 | −5.318 | 51.769 | 1.00 | 32.83 |
| ATOM | 214 | N | LYS | 39 | 34.541 | −7.360 | 51.947 | 1.00 | 31.94 |
| ATOM | 215 | CA | LYS | 39 | 33.965 | −7.343 | 50.611 | 1.00 | 33.33 |
| ATOM | 216 | CB | LYS | 39 | 33.515 | −8.754 | 50.220 | 1.00 | 34.32 |
| ATOM | 217 | CG | LYS | 39 | 33.757 | −9.105 | 48.756 | 1.00 | 41.05 |
| ATOM | 218 | CD | LYS | 39 | 32.994 | −8.183 | 47.799 | 1.00 | 43.55 |
| ATOM | 219 | CE | LYS | 39 | 33.319 | −8.502 | 46.336 | 1.00 | 47.30 |
| ATOM | 220 | NZ | LYS | 39 | 32.587 | −7.625 | 45.363 | 1.00 | 48.42 |
| ATOM | 221 | C | LYS | 39 | 32.774 | −6.378 | 50.555 | 1.00 | 32.37 |
| ATOM | 222 | O | LYS | 39 | 32.578 | −5.676 | 49.564 | 1.00 | 33.02 |
| ATOM | 223 | N | GLU | 40 | 31.975 | −6.342 | 51.613 | 1.00 | 31.82 |
| ATOM | 224 | CA | GLU | 40 | 30.831 | −5.442 | 51.632 | 1.00 | 33.50 |
| ATOM | 225 | CB | GLU | 40 | 29.845 | −5.831 | 52.737 | 1.00 | 34.39 |
| ATOM | 226 | CG | GLU | 40 | 29.159 | −7.167 | 52.507 | 1.00 | 36.32 |
| ATOM | 227 | CD | GLU | 40 | 28.562 | −7.293 | 51.112 | 1.00 | 38.53 |
| ATOM | 228 | OE1 | GLU | 40 | 27.878 | −6.350 | 50.660 | 1.00 | 39.61 |
| ATOM | 229 | OE2 | GLU | 40 | 28.770 | −8.342 | 50.469 | 1.00 | 38.22 |
| ATOM | 230 | C | GLU | 40 | 31.309 | −4.009 | 51.833 | 1.00 | 33.20 |
| ATOM | 231 | O | GLU | 40 | 30.691 | −3.072 | 51.345 | 1.00 | 33.12 |
| ATOM | 232 | N | MET | 41 | 32.409 | −3.844 | 52.556 | 1.00 | 33.18 |
| ATOM | 233 | CA | MET | 41 | 32.957 | −2.515 | 52.783 | 1.00 | 34.90 |
| ATOM | 234 | CB | MET | 41 | 34.173 | −2.585 | 53.706 | 1.00 | 32.91 |
| ATOM | 235 | CG | MET | 41 | 33.838 | −2.927 | 55.154 | 1.00 | 34.83 |
| ATOM | 236 | SD | MET | 41 | 35.327 | −2.987 | 56.170 | 1.00 | 34.41 |
| ATOM | 237 | CE | MET | 41 | 35.747 | −1.216 | 56.267 | 1.00 | 36.69 |
| ATOM | 238 | C | MET | 41 | 33.368 | −1.941 | 51.430 | 1.00 | 36.56 |
| ATOM | 239 | O | MET | 41 | 33.058 | −0.792 | 51.108 | 1.00 | 34.98 |
| ATOM | 240 | N | ASP | 42 | 34.054 | −2.758 | 50.639 | 1.00 | 36.46 |
| ATOM | 241 | CA | ASP | 42 | 34.508 | −2.346 | 49.317 | 1.00 | 38.91 |
| ATOM | 242 | CB | ASP | 42 | 35.318 | −3.470 | 48.674 | 1.00 | 42.09 |
| ATOM | 243 | CG | ASP | 42 | 36.130 | −2.999 | 47.490 | 1.00 | 43.40 |
| ATOM | 244 | OD1 | ASP | 42 | 37.081 | −2.216 | 47.705 | 1.00 | 45.67 |
| ATOM | 245 | OD2 | ASP | 42 | 35.817 | −3.411 | 46.350 | 1.00 | 42.51 |
| ATOM | 246 | C | ASP | 42 | 33.311 | −1.990 | 48.433 | 1.00 | 38.61 |
| ATOM | 247 | O | ASP | 42 | 33.366 | −1.036 | 47.656 | 1.00 | 39.03 |
| ATOM | 248 | N | ARG | 43 | 32.232 | −2.761 | 48.559 | 1.00 | 36.74 |
| ATOM | 249 | CA | ARG | 43 | 31.012 | −2.524 | 47.788 | 1.00 | 33.90 |
| ATOM | 250 | CB | ARG | 43 | 30.037 | −3.688 | 47.967 | 1.00 | 33.80 |
| ATOM | 251 | CG | ARG | 43 | 30.324 | −4.890 | 47.080 | 1.00 | 34.68 |
| ATOM | 252 | CD | ARG | 43 | 29.654 | −6.163 | 47.614 | 1.00 | 34.89 |
| ATOM | 253 | NE | ARG | 43 | 28.232 | −5.997 | 47.906 | 1.00 | 35.11 |
| ATOM | 254 | CZ | ARG | 43 | 27.296 | −5.729 | 46.998 | 1.00 | 37.42 |
| ATOM | 255 | NH1 | ARG | 43 | 27.620 | −5.589 | 45.719 | 1.00 | 39.98 |
| ATOM | 256 | NH2 | ARG | 43 | 26.028 | −5.615 | 47.366 | 1.00 | 36.46 |
| ATOM | 257 | C | ARG | 43 | 30.313 | −1.229 | 48.193 | 1.00 | 34.64 |
| ATOM | 258 | O | ARG | 43 | 29.712 | −0.550 | 47.357 | 1.00 | 35.89 |
| ATOM | 259 | N | GLY | 44 | 30.382 | −0.892 | 49.475 | 1.00 | 31.21 |
| ATOM | 260 | CA | GLY | 44 | 29.744 | 0.318 | 49.940 | 1.00 | 31.87 |
| ATOM | 261 | C | GLY | 44 | 30.463 | 1.579 | 49.490 | 1.00 | 33.29 |
| ATOM | 262 | O | GLY | 44 | 29.854 | 2.645 | 49.397 | 1.00 | 31.49 |
| ATOM | 263 | N | LEU | 45 | 31.756 | 1.455 | 49.200 | 1.00 | 31.44 |
| ATOM | 264 | CA | LEU | 45 | 32.563 | 2.595 | 48.778 | 1.00 | 32.24 |
| ATOM | 265 | CB | LEU | 45 | 34.033 | 2.358 | 49.129 | 1.00 | 27.43 |
| ATOM | 266 | CG | LEU | 45 | 34.415 | 2.487 | 50.601 | 1.00 | 29.59 |
| ATOM | 267 | CD1 | LEU | 45 | 35.832 | 1.992 | 50.827 | 1.00 | 30.31 |
| ATOM | 268 | CD2 | LEU | 45 | 34.281 | 3.941 | 51.022 | 1.00 | 30.45 |
| ATOM | 269 | C | LEU | 45 | 32.455 | 2.933 | 47.294 | 1.00 | 33.00 |
| ATOM | 270 | O | LEU | 45 | 32.537 | 4.098 | 46.924 | 1.00 | 32.78 |
| ATOM | 271 | N | ARG | 46 | 32.277 | 1.911 | 46.460 | 1.00 | 34.18 |
| ATOM | 272 | CA | ARG | 46 | 32.179 | 2.074 | 45.009 | 1.00 | 34.76 |
| ATOM | 273 | CB | ARG | 46 | 32.320 | 0.714 | 44.312 | 1.00 | 36.33 |
| ATOM | 274 | CG | ARG | 46 | 33.519 | −0.119 | 44.756 | 1.00 | 39.02 |
| ATOM | 275 | CD | ARG | 46 | 34.794 | 0.267 | 44.035 | 1.00 | 43.71 |
| ATOM | 276 | NE | ARG | 46 | 35.913 | −0.593 | 44.431 | 1.00 | 48.60 |
| ATOM | 277 | CZ | ARG | 46 | 37.142 | −0.527 | 43.915 | 1.00 | 49.59 |
| ATOM | 278 | NH1 | ARG | 46 | 37.429 | 0.359 | 42.969 | 1.00 | 49.57 |
| ATOM | 279 | NH2 | ARG | 46 | 38.091 | −1.344 | 44.354 | 1.00 | 50.09 |
| ATOM | 280 | C | ARG | 46 | 30.856 | 2.710 | 44.587 | 1.00 | 34.95 |
| ATOM | 281 | O | ARG | 46 | 29.785 | 2.361 | 45.091 | 1.00 | 32.49 |
| ATOM | 282 | N | LEU | 47 | 30.935 | 3.638 | 43.644 | 1.00 | 34.90 |
| ATOM | 283 | CA | LEU | 47 | 29.741 | 4.311 | 43.162 | 1.00 | 34.40 |
| ATOM | 284 | CB | LEU | 47 | 30.100 | 5.297 | 42.049 | 1.00 | 34.27 |
| ATOM | 285 | CG | LEU | 47 | 28.929 | 6.085 | 41.447 | 1.00 | 33.85 |
| ATOM | 286 | CD1 | LEU | 47 | 28.445 | 7.144 | 42.442 | 1.00 | 31.01 |
| ATOM | 287 | CD2 | LEU | 47 | 29.381 | 6.741 | 40.144 | 1.00 | 31.08 |
| ATOM | 288 | C | LEU | 47 | 28.727 | 3.316 | 42.625 | 1.00 | 34.52 |
| ATOM | 289 | O | LEU | 47 | 27.535 | 3.411 | 42.922 | 1.00 | 32.39 |
| ATOM | 290 | N | GLU | 48 | 29.202 | 2.353 | 41.841 | 1.00 | 34.67 |
| ATOM | 291 | CA | GLU | 48 | 28.301 | 1.378 | 41.242 | 1.00 | 36.59 |
| ATOM | 292 | CB | GLU | 48 | 29.010 | 0.589 | 40.134 | 1.00 | 38.07 |
| ATOM | 293 | CG | GLU | 48 | 30.205 | −0.248 | 40.562 | 1.00 | 39.26 |
| ATOM | 294 | CD | GLU | 48 | 31.499 | 0.534 | 40.580 | 1.00 | 40.85 |
| ATOM | 295 | OE1 | GLU | 48 | 32.571 | −0.106 | 40.497 | 1.00 | 44.46 |
| ATOM | 296 | OE2 | GLU | 48 | 31.454 | 1.779 | 40.682 | 1.00 | 38.21 |
| ATOM | 297 | C | GLU | 48 | 27.600 | 0.406 | 42.188 | 1.00 | 37.46 |
| ATOM | 298 | O | GLU | 48 | 26.654 | −0.268 | 41.778 | 1.00 | 37.82 |
| ATOM | 299 | N | THR | 49 | 28.037 | 0.321 | 43.441 | 1.00 | 36.85 |
| ATOM | 300 | CA | THR | 49 | 27.371 | −0.591 | 44.370 | 1.00 | 36.40 |
| ATOM | 301 | CB | THR | 49 | 28.212 | −1.855 | 44.645 | 1.00 | 34.37 |
| ATOM | 302 | OG1 | THR | 49 | 29.554 | −1.480 | 44.969 | 1.00 | 33.33 |
| ATOM | 303 | CG2 | THR | 49 | 28.215 | −2.770 | 43.437 | 1.00 | 32.44 |
| ATOM | 304 | C | THR | 49 | 27.032 | 0.037 | 45.703 | 1.00 | 38.54 |
| ATOM | 305 | O | THR | 49 | 26.536 | −0.647 | 46.599 | 1.00 | 40.86 |
| ATOM | 306 | N | HIS | 50 | 27.272 | 1.335 | 45.842 | 1.00 | 38.89 |
| ATOM | 307 | CA | HIS | 50 | 26.994 | 1.990 | 47.115 | 1.00 | 41.74 |
| ATOM | 308 | CB | HIS | 50 | 27.548 | 3.422 | 47.130 | 1.00 | 44.04 |
| ATOM | 309 | CG | HIS | 50 | 26.666 | 4.426 | 46.451 | 1.00 | 46.35 |
| ATOM | 310 | CD2 | HIS | 50 | 25.795 | 5.331 | 46.959 | 1.00 | 48.65 |
| ATOM | 311 | ND1 | HIS | 50 | 26.607 | 4.565 | 45.081 | 1.00 | 47.18 |
| ATOM | 312 | CE1 | HIS | 50 | 25.738 | 5.512 | 44.772 | 1.00 | 48.13 |
| ATOM | 313 | NE2 | HIS | 50 | 25.231 | 5.993 | 45.894 | 1.00 | 49.20 |
| ATOM | 314 | C | HIS | 50 | 25.512 | 2.030 | 47.466 | 1.00 | 42.66 |
| ATOM | 315 | O | HIS | 50 | 25.153 | 2.046 | 48.642 | 1.00 | 42.85 |
| ATOM | 316 | N | GLU | 51 | 24.657 | 2.034 | 46.447 | 1.00 | 43.12 |
| ATOM | 317 | CA | GLU | 51 | 23.213 | 2.120 | 46.645 | 1.00 | 44.07 |
| ATOM | 318 | CB | GLU | 51 | 22.555 | 2.574 | 45.329 | 1.00 | 44.83 |
| ATOM | 319 | CG | GLU | 51 | 21.051 | 2.824 | 45.399 | 1.00 | 46.43 |
| ATOM | 320 | CD | GLU | 51 | 20.531 | 3.691 | 44.243 | 1.00 | 48.89 |
| ATOM | 321 | OE1 | GLU | 51 | 20.822 | 3.385 | 43.064 | 1.00 | 46.31 |
| ATOM | 322 | OE2 | GLU | 51 | 19.821 | 4.683 | 44.522 | 1.00 | 50.83 |
| ATOM | 323 | C | GLU | 51 | 22.543 | 0.848 | 47.179 | 1.00 | 44.27 |
| ATOM | 324 | O | GLU | 51 | 21.630 | 0.925 | 48.000 | 1.00 | 45.14 |
| ATOM | 325 | N | GLU | 52 | 22.991 | −0.317 | 46.723 | 1.00 | 44.47 |
| ATOM | 326 | CA | GLU | 52 | 22.422 | −1.585 | 47.178 | 1.00 | 44.81 |
| ATOM | 327 | CB | GLU | 52 | 22.199 | −2.521 | 45.988 | 1.00 | 47.15 |
| ATOM | 328 | CG | GLU | 52 | 23.485 | −2.920 | 45.264 | 1.00 | 53.66 |
| ATOM | 329 | CD | GLU | 52 | 23.698 | −2.164 | 43.951 | 1.00 | 57.63 |
| ATOM | 330 | OE1 | GLU | 52 | 23.646 | −0.909 | 43.953 | 1.00 | 55.90 |
| ATOM | 331 | OE2 | GLU | 52 | 23.925 | −2.835 | 42.917 | 1.00 | 57.72 |
| ATOM | 332 | C | GLU | 52 | 23.313 | −2.297 | 48.206 | 1.00 | 42.49 |
| ATOM | 333 | O | GLU | 52 | 23.052 | −3.441 | 48.575 | 1.00 | 43.45 |
| ATOM | 334 | N | ALA | 53 | 24.362 | −1.626 | 48.666 | 1.00 | 39.72 |
| ATOM | 335 | CA | ALA | 53 | 25.285 | −2.224 | 49.628 | 1.00 | 37.01 |
| ATOM | 336 | CB | ALA | 53 | 26.589 | −1.438 | 49.645 | 1.00 | 35.23 |
| ATOM | 337 | C | ALA | 53 | 24.700 | −2.291 | 51.038 | 1.00 | 35.27 |
| ATOM | 338 | O | ALA | 53 | 24.125 | −1.321 | 51.528 | 1.00 | 34.63 |
| ATOM | 339 | N | SER | 54 | 24.845 | −3.439 | 51.689 | 1.00 | 32.88 |
| ATOM | 340 | CA | SER | 54 | 24.339 | −3.594 | 53.052 | 1.00 | 32.06 |
| ATOM | 341 | CB | SER | 54 | 24.397 | −5.062 | 53.476 | 1.00 | 30.23 |
| ATOM | 342 | OG | SER | 54 | 25.694 | −5.576 | 53.261 | 1.00 | 35.67 |
| ATOM | 343 | C | SER | 54 | 25.188 | −2.741 | 53.990 | 1.00 | 28.49 |
| ATOM | 344 | O | SER | 54 | 24.682 | −2.147 | 54.934 | 1.00 | 29.57 |
| ATOM | 345 | N | VAL | 55 | 26.485 | −2.684 | 53.724 | 1.00 | 28.44 |
| ATOM | 346 | CA | VAL | 55 | 27.386 | −1.876 | 54.535 | 1.00 | 28.63 |
| ATOM | 347 | CB | VAL | 55 | 28.737 | −2.594 | 54.726 | 1.00 | 27.89 |
| ATOM | 348 | CG1 | VAL | 55 | 29.660 | −1.766 | 55.599 | 1.00 | 26.89 |
| ATOM | 349 | CG2 | VAL | 55 | 28.497 | −3.957 | 55.365 | 1.00 | 27.94 |
| ATOM | 350 | C | VAL | 55 | 27.559 | −0.551 | 53.788 | 1.00 | 29.80 |
| ATOM | 351 | O | VAL | 55 | 28.367 | −0.430 | 52.868 | 1.00 | 28.14 |
| ATOM | 352 | N | LYS | 56 | 26.787 | 0.446 | 54.205 | 1.00 | 31.68 |
| ATOM | 353 | CA | LYS | 56 | 26.788 | 1.750 | 53.550 | 1.00 | 30.06 |
| ATOM | 354 | CB | LYS | 56 | 25.727 | 2.628 | 54.203 | 1.00 | 29.96 |
| ATOM | 355 | CG | LYS | 56 | 24.312 | 2.124 | 53.933 | 1.00 | 29.47 |
| ATOM | 356 | CD | LYS | 56 | 23.279 | 2.935 | 54.689 | 1.00 | 31.68 |
| ATOM | 357 | CE | LYS | 56 | 23.417 | 2.767 | 56.196 | 1.00 | 30.78 |
| ATOM | 358 | NZ | LYS | 56 | 22.911 | 1.428 | 56.648 | 1.00 | 36.66 |
| ATOM | 359 | C | LYS | 56 | 28.087 | 2.535 | 53.374 | 1.00 | 28.33 |
| ATOM | 360 | O | LYS | 56 | 28.222 | 3.256 | 52.388 | 1.00 | 30.83 |
| ATOM | 361 | N | MET | 57 | 29.044 | 2.410 | 54.287 | 1.00 | 25.97 |
| ATOM | 362 | CA | MET | 57 | 30.299 | 3.149 | 54.137 | 1.00 | 23.92 |
| ATOM | 363 | CB | MET | 57 | 31.098 | 2.577 | 52.964 | 1.00 | 24.05 |
| ATOM | 364 | CG | MET | 57 | 31.383 | 1.078 | 53.075 | 1.00 | 27.54 |
| ATOM | 365 | SD | MET | 57 | 32.303 | 0.659 | 54.580 | 1.00 | 26.48 |
| ATOM | 366 | CE | MET | 57 | 33.991 | 1.127 | 54.113 | 1.00 | 21.76 |
| ATOM | 367 | C | MET | 57 | 30.006 | 4.643 | 53.887 | 1.00 | 26.44 |
| ATOM | 368 | O | MET | 57 | 30.460 | 5.237 | 52.903 | 1.00 | 24.39 |
| ATOM | 369 | N | LEU | 58 | 29.250 | 5.235 | 54.803 | 1.00 | 26.42 |
| ATOM | 370 | CA | LEU | 58 | 28.843 | 6.630 | 54.713 | 1.00 | 26.83 |
| ATOM | 371 | CB | LEU | 58 | 27.684 | 6.884 | 55.677 | 1.00 | 24.27 |
| ATOM | 372 | CG | LEU | 58 | 26.440 | 6.043 | 55.386 | 1.00 | 30.26 |
| ATOM | 373 | CD1 | LEU | 58 | 25.401 | 6.250 | 56.473 | 1.00 | 28.51 |
| ATOM | 374 | CD2 | LEU | 58 | 25.874 | 6.430 | 54.016 | 1.00 | 31.10 |
| ATOM | 375 | C | LEU | 58 | 29.932 | 7.665 | 54.965 | 1.00 | 25.48 |
| ATOM | 376 | O | LEU | 58 | 30.495 | 7.742 | 56.053 | 1.00 | 25.30 |
| ATOM | 377 | N | PRO | 59 | 30.242 | 8.476 | 53.946 | 1.00 | 24.56 |
| ATOM | 378 | CD | PRO | 59 | 29.764 | 8.341 | 52.557 | 1.00 | 24.76 |
| ATOM | 379 | CA | PRO | 59 | 31.262 | 9.528 | 54.063 | 1.00 | 26.48 |
| ATOM | 380 | CB | PRO | 59 | 31.217 | 10.196 | 52.686 | 1.00 | 26.76 |
| ATOM | 381 | CG | PRO | 59 | 30.865 | 9.036 | 51.769 | 1.00 | 26.41 |
| ATOM | 382 | C | PRO | 59 | 30.820 | 10.478 | 55.190 | 1.00 | 26.49 |
| ATOM | 383 | O | PRO | 59 | 29.656 | 10.863 | 55.239 | 1.00 | 28.20 |
| ATOM | 384 | N | THR | 60 | 31.728 | 10.845 | 56.092 | 1.00 | 27.28 |
| ATOM | 385 | CA | THR | 60 | 31.372 | 11.720 | 57.220 | 1.00 | 27.77 |
| ATOM | 386 | CB | THR | 60 | 31.994 | 11.217 | 58.544 | 1.00 | 24.87 |
| ATOM | 387 | OG1 | THR | 60 | 33.400 | 11.482 | 58.536 | 1.00 | 22.66 |
| ATOM | 388 | CG2 | THR | 60 | 31.767 | 9.713 | 58.726 | 1.00 | 28.80 |
| ATOM | 389 | C | THR | 60 | 31.800 | 13.196 | 57.085 | 1.00 | 30.72 |
| ATOM | 390 | O | THR | 60 | 31.405 | 14.041 | 57.897 | 1.00 | 29.67 |
| ATOM | 391 | N | TYR | 61 | 32.623 | 13.485 | 56.084 | 1.00 | 30.13 |
| ATOM | 392 | CA | TYR | 61 | 33.144 | 14.824 | 55.844 | 1.00 | 33.87 |
| ATOM | 393 | CB | TYR | 61 | 32.005 | 15.837 | 55.684 | 1.00 | 32.96 |
| ATOM | 394 | CG | TYR | 61 | 31.409 | 15.730 | 54.298 | 1.00 | 35.37 |
| ATOM | 395 | CD1 | TYR | 61 | 32.084 | 16.251 | 53.192 | 1.00 | 36.43 |
| ATOM | 396 | CE1 | TYR | 61 | 31.621 | 16.036 | 51.890 | 1.00 | 34.05 |
| ATOM | 397 | CD2 | TYR | 61 | 30.244 | 14.995 | 54.068 | 1.00 | 34.99 |
| ATOM | 398 | CE2 | TYR | 61 | 29.778 | 14.772 | 52.768 | 1.00 | 33.96 |
| ATOM | 399 | CZ | TYR | 61 | 30.475 | 15.294 | 51.689 | 1.00 | 33.72 |
| ATOM | 400 | OH | TYR | 61 | 30.039 | 15.064 | 50.402 | 1.00 | 37.69 |
| ATOM | 401 | C | TYR | 61 | 34.156 | 15.264 | 56.890 | 1.00 | 34.78 |
| ATOM | 402 | O | TYR | 61 | 34.712 | 16.357 | 56.806 | 1.00 | 34.09 |
| ATOM | 403 | N | VAL | 62 | 34.407 | 14.407 | 57.875 | 1.00 | 36.47 |
| ATOM | 404 | CA | VAL | 62 | 35.426 | 14.713 | 58.869 | 1.00 | 37.40 |
| ATOM | 405 | CB | VAL | 62 | 35.283 | 13.825 | 60.116 | 1.00 | 37.42 |
| ATOM | 406 | CG1 | VAL | 62 | 36.410 | 14.107 | 61.089 | 1.00 | 32.97 |
| ATOM | 407 | CG2 | VAL | 62 | 33.937 | 14.073 | 60.774 | 1.00 | 36.34 |
| ATOM | 408 | C | VAL | 62 | 36.695 | 14.335 | 58.104 | 1.00 | 41.04 |
| ATOM | 409 | O | VAL | 62 | 36.944 | 13.153 | 57.865 | 1.00 | 40.85 |
| ATOM | 410 | N | ARG | 63 | 37.475 | 15.331 | 57.692 | 1.00 | 43.48 |
| ATOM | 411 | CA | ARG | 63 | 38.682 | 15.070 | 56.909 | 1.00 | 48.27 |
| ATOM | 412 | CB | ARG | 63 | 38.843 | 16.126 | 55.814 | 1.00 | 47.25 |
| ATOM | 413 | CG | ARG | 63 | 37.735 | 16.112 | 54.783 | 1.00 | 49.66 |
| ATOM | 414 | CD | ARG | 63 | 37.648 | 17.447 | 54.061 | 1.00 | 50.62 |
| ATOM | 415 | NE | ARG | 63 | 36.482 | 17.523 | 53.185 | 1.00 | 51.28 |
| ATOM | 416 | CZ | ARG | 63 | 36.405 | 16.961 | 51.982 | 1.00 | 50.52 |
| ATOM | 417 | NH1 | ARG | 63 | 37.430 | 16.274 | 51.492 | 1.00 | 48.44 |
| ATOM | 418 | NH2 | ARG | 63 | 35.295 | 17.089 | 51.268 | 1.00 | 49.50 |
| ATOM | 419 | C | ARG | 63 | 39.952 | 15.006 | 57.728 | 1.00 | 50.30 |
| ATOM | 420 | O | ARG | 63 | 39.998 | 15.478 | 58.860 | 1.00 | 49.69 |
| ATOM | 421 | N | SER | 64 | 40.987 | 14.431 | 57.128 | 1.00 | 54.64 |
| ATOM | 422 | CA | SER | 64 | 42.276 | 14.280 | 57.783 | 1.00 | 60.87 |
| ATOM | 423 | CB | SER | 64 | 43.315 | 13.760 | 56.794 | 1.00 | 60.13 |
| ATOM | 424 | OG | SER | 64 | 44.492 | 13.381 | 57.481 | 1.00 | 62.83 |
| ATOM | 425 | C | SER | 64 | 42.760 | 15.583 | 58.398 | 1.00 | 65.69 |
| ATOM | 426 | O | SER | 64 | 42.952 | 16.584 | 57.703 | 1.00 | 65.99 |
| ATOM | 427 | N | THR | 65 | 42.961 | 15.530 | 59.714 | 1.00 | 71.92 |
| ATOM | 428 | CA | THR | 65 | 43.402 | 16.649 | 60.545 | 1.00 | 77.78 |
| ATOM | 429 | CB | THR | 65 | 44.529 | 16.194 | 61.524 | 1.00 | 78.35 |
| ATOM | 430 | OG1 | THR | 65 | 44.959 | 17.309 | 62.317 | 1.00 | 79.07 |
| ATOM | 431 | CG2 | THR | 65 | 45.714 | 15.611 | 60.757 | 1.00 | 79.19 |
| ATOM | 432 | C | THR | 65 | 43.839 | 17.925 | 59.817 | 1.00 | 80.90 |
| ATOM | 433 | O | THR | 65 | 45.033 | 18.188 | 59.654 | 1.00 | 80.93 |
| ATOM | 434 | N | PRO | 66 | 42.863 | 18.732 | 59.364 | 1.00 | 83.72 |
| ATOM | 435 | CD | PRO | 66 | 41.410 | 18.469 | 59.372 | 1.00 | 84.56 |
| ATOM | 436 | CA | PRO | 66 | 43.162 | 19.983 | 58.661 | 1.00 | 85.58 |
| ATOM | 437 | CB | PRO | 66 | 41.871 | 20.254 | 57.897 | 1.00 | 85.53 |
| ATOM | 438 | CG | PRO | 66 | 40.827 | 19.776 | 58.864 | 1.00 | 85.36 |
| ATOM | 439 | C | PRO | 66 | 43.468 | 21.057 | 59.710 | 1.00 | 87.07 |
| ATOM | 440 | O | PRO | 66 | 42.581 | 21.812 | 60.119 | 1.00 | 87.87 |
| ATOM | 441 | N | GLU | 67 | 44.726 | 21.109 | 60.144 | 1.00 | 87.71 |
| ATOM | 442 | CA | GLU | 67 | 45.162 | 22.055 | 61.169 | 1.00 | 87.66 |
| ATOM | 443 | CB | GLU | 67 | 46.683 | 22.238 | 61.110 | 1.00 | 88.42 |
| ATOM | 444 | CG | GLU | 67 | 47.283 | 22.824 | 62.384 | 1.00 | 89.15 |
| ATOM | 445 | CD | GLU | 67 | 46.871 | 22.058 | 63.636 | 1.00 | 89.71 |
| ATOM | 446 | OE1 | GLU | 67 | 45.689 | 22.150 | 64.037 | 1.00 | 89.95 |
| ATOM | 447 | OE2 | GLU | 67 | 47.728 | 21.359 | 64.217 | 1.00 | 89.51 |
| ATOM | 448 | C | GLU | 67 | 44.463 | 23.413 | 61.095 | 1.00 | 86.97 |
| ATOM | 449 | O | GLU | 67 | 44.203 | 23.944 | 60.013 | 1.00 | 86.95 |
| ATOM | 450 | N | GLY | 68 | 44.160 | 23.962 | 62.266 | 1.00 | 85.72 |
| ATOM | 451 | CA | GLY | 68 | 43.475 | 25.237 | 62.344 | 1.00 | 83.56 |
| ATOM | 452 | C | GLY | 68 | 42.274 | 25.073 | 63.251 | 1.00 | 82.01 |
| ATOM | 453 | O | GLY | 68 | 41.136 | 24.970 | 62.784 | 1.00 | 82.39 |
| ATOM | 454 | N | SER | 69 | 42.530 | 25.038 | 64.555 | 1.00 | 79.39 |
| ATOM | 455 | CA | SER | 69 | 41.469 | 24.869 | 65.537 | 1.00 | 77.31 |
| ATOM | 456 | CB | SER | 69 | 41.855 | 23.784 | 66.542 | 1.00 | 77.69 |
| ATOM | 457 | OG | SER | 69 | 40.877 | 23.677 | 67.561 | 1.00 | 78.20 |
| ATOM | 458 | C | SER | 69 | 41.118 | 26.143 | 66.294 | 1.00 | 75.21 |
| ATOM | 459 | O | SER | 69 | 41.993 | 26.857 | 66.784 | 1.00 | 74.23 |
| ATOM | 460 | N | GLU | 70 | 39.822 | 26.413 | 66.386 | 1.00 | 73.26 |
| ATOM | 461 | CA | GLU | 70 | 39.328 | 27.581 | 67.096 | 1.00 | 71.89 |
| ATOM | 462 | CB | GLU | 70 | 38.004 | 28.042 | 66.482 | 1.00 | 73.40 |
| ATOM | 463 | CG | GLU | 70 | 37.897 | 29.544 | 66.297 | 1.00 | 77.84 |
| ATOM | 464 | CD | GLU | 70 | 38.900 | 30.073 | 65.285 | 1.00 | 80.27 |
| ATOM | 465 | OE1 | GLU | 70 | 38.763 | 29.757 | 64.082 | 1.00 | 81.41 |
| ATOM | 466 | OE2 | GLU | 70 | 39.830 | 30.801 | 65.692 | 1.00 | 81.33 |
| ATOM | 467 | C | GLU | 70 | 39.107 | 27.144 | 68.543 | 1.00 | 69.48 |
| ATOM | 468 | O | GLU | 70 | 38.409 | 26.163 | 68.789 | 1.00 | 69.73 |
| ATOM | 469 | N | VAL | 71 | 39.701 | 27.853 | 69.499 | 1.00 | 65.92 |
| ATOM | 470 | CA | VAL | 71 | 39.536 | 27.490 | 70.904 | 1.00 | 62.64 |
| ATOM | 471 | CB | VAL | 71 | 40.760 | 27.909 | 71.746 | 1.00 | 61.59 |
| ATOM | 472 | CG1 | VAL | 71 | 41.993 | 27.156 | 71.275 | 1.00 | 61.91 |
| ATOM | 473 | CG2 | VAL | 71 | 40.979 | 29.406 | 71.642 | 1.00 | 61.78 |
| ATOM | 474 | C | VAL | 71 | 38.278 | 28.105 | 71.510 | 1.00 | 61.05 |
| ATOM | 475 | O | VAL | 71 | 37.608 | 28.919 | 70.877 | 1.00 | 61.02 |
| ATOM | 476 | N | GLY | 72 | 37.952 | 27.700 | 72.734 | 1.00 | 59.60 |
| ATOM | 477 | CA | GLY | 72 | 36.769 | 28.225 | 73.390 | 1.00 | 58.10 |
| ATOM | 478 | C | GLY | 72 | 35.841 | 27.169 | 73.967 | 1.00 | 57.74 |
| ATOM | 479 | O | GLY | 72 | 36.178 | 25.982 | 74.006 | 1.00 | 58.27 |
| ATOM | 480 | N | ASP | 73 | 34.664 | 27.607 | 74.410 | 1.00 | 55.55 |
| ATOM | 481 | CA | ASP | 73 | 33.663 | 26.724 | 75.003 | 1.00 | 54.21 |
| ATOM | 482 | CB | ASP | 73 | 32.973 | 27.426 | 76.181 | 1.00 | 57.20 |
| ATOM | 483 | CG | ASP | 73 | 33.846 | 27.496 | 77.424 | 1.00 | 59.78 |
| ATOM | 484 | OD1 | ASP | 73 | 35.046 | 27.830 | 77.299 | 1.00 | 61.37 |
| ATOM | 485 | OD2 | ASP | 73 | 33.324 | 27.225 | 78.529 | 1.00 | 60.87 |
| ATOM | 486 | C | ASP | 73 | 32.599 | 26.310 | 73.994 | 1.00 | 52.36 |
| ATOM | 487 | O | ASP | 73 | 31.936 | 27.161 | 73.406 | 1.00 | 52.44 |
| ATOM | 488 | N | PHE | 74 | 32.424 | 25.005 | 73.800 | 1.00 | 49.73 |
| ATOM | 489 | CA | PHE | 74 | 31.412 | 24.519 | 72.866 | 1.00 | 46.98 |
| ATOM | 490 | CB | PHE | 74 | 32.019 | 23.571 | 71.837 | 1.00 | 46.41 |
| ATOM | 491 | CG | PHE | 74 | 33.117 | 24.179 | 71.030 | 1.00 | 47.09 |
| ATOM | 492 | CD1 | PHE | 74 | 34.335 | 24.492 | 71.618 | 1.00 | 47.62 |
| ATOM | 493 | CD2 | PHE | 74 | 32.930 | 24.452 | 69.681 | 1.00 | 47.01 |
| ATOM | 494 | CE1 | PHE | 74 | 35.359 | 25.071 | 70.874 | 1.00 | 49.47 |
| ATOM | 495 | CE2 | PHE | 74 | 33.943 | 25.031 | 68.924 | 1.00 | 48.12 |
| ATOM | 496 | CZ | PHE | 74 | 35.161 | 25.342 | 69.520 | 1.00 | 48.82 |
| ATOM | 497 | C | PHE | 74 | 30.316 | 23.783 | 73.601 | 1.00 | 45.68 |
| ATOM | 498 | O | PHE | 74 | 30.485 | 23.382 | 74.745 | 1.00 | 46.35 |
| ATOM | 499 | N | LEU | 75 | 29.185 | 23.615 | 72.932 | 1.00 | 45.12 |
| ATOM | 500 | CA | LEU | 75 | 28.064 | 22.895 | 73.501 | 1.00 | 44.80 |
| ATOM | 501 | CB | LEU | 75 | 26.769 | 23.686 | 73.333 | 1.00 | 43.29 |
| ATOM | 502 | CG | LEU | 75 | 25.535 | 23.023 | 73.959 | 1.00 | 45.05 |
| ATOM | 503 | CD1 | LEU | 75 | 25.529 | 23.278 | 75.466 | 1.00 | 41.53 |
| ATOM | 504 | CD2 | LEU | 75 | 24.259 | 23.571 | 73.326 | 1.00 | 43.45 |
| ATOM | 505 | C | LEU | 75 | 27.971 | 21.598 | 72.708 | 1.00 | 46.04 |
| ATOM | 506 | O | LEU | 75 | 28.087 | 21.611 | 71.479 | 1.00 | 46.97 |
| ATOM | 507 | N | SER | 76 | 27.770 | 20.484 | 73.405 | 1.00 | 45.48 |
| ATOM | 508 | CA | SER | 76 | 27.664 | 19.189 | 72.744 | 1.00 | 43.73 |
| ATOM | 509 | CB | SER | 76 | 28.837 | 18.295 | 73.143 | 1.00 | 43.52 |
| ATOM | 510 | OG | SER | 76 | 30.040 | 18.741 | 72.551 | 1.00 | 44.64 |
| ATOM | 511 | C | SER | 76 | 26.361 | 18.469 | 73.051 | 1.00 | 41.60 |
| ATOM | 512 | O | SER | 76 | 26.026 | 18.242 | 74.209 | 1.00 | 40.88 |
| ATOM | 513 | N | LEU | 77 | 25.617 | 18.130 | 72.007 | 1.00 | 41.06 |
| ATOM | 514 | CA | LEU | 77 | 24.369 | 17.397 | 72.175 | 1.00 | 43.50 |
| ATOM | 515 | CB | LEU | 77 | 23.281 | 17.918 | 71.225 | 1.00 | 43.84 |
| ATOM | 516 | CG | LEU | 77 | 22.750 | 19.346 | 71.401 | 1.00 | 45.70 |
| ATOM | 517 | CD1 | LEU | 77 | 21.587 | 19.577 | 70.442 | 1.00 | 45.96 |
| ATOM | 518 | CD2 | LEU | 77 | 22.284 | 19.550 | 72.835 | 1.00 | 46.75 |
| ATOM | 519 | C | LEU | 77 | 24.662 | 15.933 | 71.851 | 1.00 | 43.78 |
| ATOM | 520 | O | LEU | 77 | 25.529 | 15.635 | 71.026 | 1.00 | 43.07 |
| ATOM | 521 | N | ASP | 78 | 23.946 | 15.021 | 72.496 | 1.00 | 44.50 |
| ATOM | 522 | CA | ASP | 78 | 24.151 | 13.604 | 72.244 | 1.00 | 44.82 |
| ATOM | 523 | CB | ASP | 78 | 25.126 | 13.026 | 73.271 | 1.00 | 44.71 |
| ATOM | 524 | CG | ASP | 78 | 25.597 | 11.628 | 72.905 | 1.00 | 45.55 |
| ATOM | 525 | OD1 | ASP | 78 | 24.738 | 10.750 | 72.672 | 1.00 | 41.76 |
| ATOM | 526 | OD2 | ASP | 78 | 26.828 | 11.410 | 72.853 | 1.00 | 45.32 |
| ATOM | 527 | C | ASP | 78 | 22.838 | 12.829 | 72.276 | 1.00 | 44.74 |
| ATOM | 528 | O | ASP | 78 | 22.245 | 12.633 | 73.333 | 1.00 | 45.25 |
| ATOM | 529 | N | LEU | 79 | 22.385 | 12.398 | 71.107 | 1.00 | 45.72 |
| ATOM | 530 | CA | LEU | 79 | 21.154 | 11.630 | 70.994 | 1.00 | 47.25 |
| ATOM | 531 | CB | LEU | 79 | 20.137 | 12.351 | 70.116 | 1.00 | 45.37 |
| ATOM | 532 | CG | LEU | 79 | 18.865 | 11.530 | 69.915 | 1.00 | 43.65 |
| ATOM | 533 | CD1 | LEU | 79 | 18.067 | 11.553 | 71.200 | 1.00 | 46.42 |
| ATOM | 534 | CD2 | LEU | 79 | 18.045 | 12.086 | 68.777 | 1.00 | 43.81 |
| ATOM | 535 | C | LEU | 79 | 21.491 | 10.295 | 70.354 | 1.00 | 49.50 |
| ATOM | 536 | O | LEU | 79 | 22.073 | 10.249 | 69.274 | 1.00 | 49.35 |
| ATOM | 537 | N | GLY | 80 | 21.123 | 9.207 | 71.016 | 1.00 | 52.24 |
| ATOM | 538 | CA | GLY | 80 | 21.421 | 7.902 | 70.466 | 1.00 | 56.31 |
| ATOM | 539 | C | GLY | 80 | 20.965 | 6.833 | 71.420 | 1.00 | 59.13 |
| ATOM | 540 | O | GLY | 80 | 20.278 | 5.896 | 71.027 | 1.00 | 60.86 |
| ATOM | 541 | N | GLY | 81 | 21.360 | 6.966 | 72.679 | 1.00 | 62.30 |
| ATOM | 542 | CA | GLY | 81 | 20.940 | 6.002 | 73.674 | 1.00 | 65.60 |
| ATOM | 543 | C | GLY | 81 | 19.551 | 6.395 | 74.137 | 1.00 | 67.84 |
| ATOM | 544 | O | GLY | 81 | 18.936 | 7.301 | 73.564 | 1.00 | 69.00 |
| ATOM | 545 | N | THR | 82 | 19.047 | 5.722 | 75.165 | 1.00 | 69.33 |
| ATOM | 546 | CA | THR | 82 | 17.726 | 6.037 | 75.695 | 1.00 | 70.36 |
| ATOM | 547 | CB | THR | 82 | 17.110 | 4.824 | 76.418 | 1.00 | 71.43 |
| ATOM | 548 | OG1 | THR | 82 | 18.032 | 4.332 | 77.398 | 1.00 | 71.60 |
| ATOM | 549 | CG2 | THR | 82 | 16.784 | 3.716 | 75.420 | 1.00 | 71.87 |
| ATOM | 550 | C | THR | 82 | 17.846 | 7.196 | 76.679 | 1.00 | 70.10 |
| ATOM | 551 | O | THR | 82 | 16.933 | 7.458 | 77.464 | 1.00 | 71.18 |
| ATOM | 552 | N | ASN | 83 | 18.981 | 7.887 | 76.625 | 1.00 | 69.08 |
| ATOM | 553 | CA | ASN | 83 | 19.232 | 9.017 | 77.508 | 1.00 | 68.14 |
| ATOM | 554 | CB | ASN | 83 | 20.161 | 8.584 | 78.646 | 1.00 | 69.98 |
| ATOM | 555 | CG | ASN | 83 | 19.862 | 9.300 | 79.948 | 1.00 | 70.80 |
| ATOM | 556 | OD1 | ASN | 83 | 20.627 | 9.213 | 80.909 | 1.00 | 71.46 |
| ATOM | 557 | ND2 | ASN | 83 | 18.739 | 10.004 | 79.990 | 1.00 | 72.56 |
| ATOM | 558 | C | ASN | 83 | 19.866 | 10.177 | 76.738 | 1.00 | 66.16 |
| ATOM | 559 | O | ASN | 83 | 21.050 | 10.136 | 76.407 | 1.00 | 66.52 |
| ATOM | 560 | N | PHE | 84 | 19.073 | 11.203 | 76.447 | 1.00 | 63.41 |
| ATOM | 561 | CA | PHE | 84 | 19.567 | 12.375 | 75.728 | 1.00 | 60.93 |
| ATOM | 562 | CB | PHE | 84 | 18.398 | 13.227 | 75.241 | 1.00 | 61.87 |
| ATOM | 563 | CG | PHE | 84 | 18.817 | 14.477 | 74.528 | 1.00 | 63.55 |
| ATOM | 564 | CD1 | PHE | 84 | 18.419 | 15.724 | 74.993 | 1.00 | 63.38 |
| ATOM | 565 | CD2 | PHE | 84 | 19.599 | 14.409 | 73.381 | 1.00 | 64.28 |
| ATOM | 566 | CE1 | PHE | 84 | 18.793 | 16.888 | 74.325 | 1.00 | 64.07 |
| ATOM | 567 | CE2 | PHE | 84 | 19.979 | 15.568 | 72.705 | 1.00 | 65.31 |
| ATOM | 568 | CZ | PHE | 84 | 19.574 | 16.810 | 73.179 | 1.00 | 64.75 |
| ATOM | 569 | C | PHE | 84 | 20.442 | 13.206 | 76.658 | 1.00 | 59.07 |
| ATOM | 570 | O | PHE | 84 | 20.011 | 13.582 | 77.744 | 1.00 | 59.19 |
| ATOM | 571 | N | ARG | 85 | 21.665 | 13.500 | 76.232 | 1.00 | 57.25 |
| ATOM | 572 | CA | ARG | 85 | 22.583 | 14.272 | 77.064 | 1.00 | 56.05 |
| ATOM | 573 | CB | ARG | 85 | 23.857 | 13.467 | 77.344 | 1.00 | 56.68 |
| ATOM | 574 | CG | ARG | 85 | 23.605 | 12.044 | 77.828 | 1.00 | 58.78 |
| ATOM | 575 | CD | ARG | 85 | 24.896 | 11.367 | 78.267 | 1.00 | 59.39 |
| ATOM | 576 | NE | ARG | 85 | 25.908 | 11.348 | 77.213 | 1.00 | 59.87 |
| ATOM | 577 | CZ | ARG | 85 | 27.068 | 11.994 | 77.282 | 1.00 | 60.09 |
| ATOM | 578 | NH1 | ARG | 85 | 27.366 | 12.713 | 78.357 | 1.00 | 59.50 |
| ATOM | 579 | NH2 | ARG | 85 | 27.931 | 11.920 | 76.277 | 1.00 | 60.92 |
| ATOM | 580 | C | ARG | 85 | 22.966 | 15.602 | 76.433 | 1.00 | 55.07 |
| ATOM | 581 | O | ARG | 85 | 23.038 | 15.725 | 75.209 | 1.00 | 54.93 |
| ATOM | 582 | N | VAL | 86 | 23.211 | 16.593 | 77.288 | 1.00 | 53.13 |
| ATOM | 583 | CA | VAL | 86 | 23.598 | 17.935 | 76.861 | 1.00 | 51.01 |
| ATOM | 584 | CB | VAL | 86 | 22.425 | 18.939 | 77.003 | 1.00 | 51.19 |
| ATOM | 585 | CG1 | VAL | 86 | 22.851 | 20.313 | 76.509 | 1.00 | 51.39 |
| ATOM | 586 | CG2 | VAL | 86 | 21.216 | 18.446 | 76.225 | 1.00 | 50.96 |
| ATOM | 587 | C | VAL | 86 | 24.734 | 18.381 | 77.767 | 1.00 | 49.34 |
| ATOM | 588 | O | VAL | 86 | 24.613 | 18.316 | 78.989 | 1.00 | 48.07 |
| ATOM | 589 | N | MET | 87 | 25.834 | 18.835 | 77.178 | 1.00 | 49.52 |
| ATOM | 590 | CA | MET | 87 | 26.970 | 19.260 | 77.981 | 1.00 | 50.78 |
| ATOM | 591 | CB | MET | 87 | 27.864 | 18.054 | 78.284 | 1.00 | 52.70 |
| ATOM | 592 | CG | MET | 87 | 28.572 | 17.461 | 77.072 | 1.00 | 54.49 |
| ATOM | 593 | SD | MET | 87 | 29.005 | 15.694 | 77.269 | 1.00 | 53.62 |
| ATOM | 594 | CE | MET | 87 | 27.839 | 14.951 | 76.090 | 1.00 | 51.63 |
| ATOM | 595 | C | MET | 87 | 27.800 | 20.363 | 77.348 | 1.00 | 50.56 |
| ATOM | 596 | O | MET | 87 | 27.715 | 20.616 | 76.149 | 1.00 | 50.18 |
| ATOM | 597 | N | LEU | 88 | 28.605 | 21.015 | 78.178 | 1.00 | 50.90 |
| ATOM | 598 | CA | LEU | 88 | 29.477 | 22.093 | 77.739 | 1.00 | 52.10 |
| ATOM | 599 | CB | LEU | 88 | 29.278 | 23.325 | 78.631 | 1.00 | 53.23 |
| ATOM | 600 | CG | LEU | 88 | 30.087 | 24.580 | 78.288 | 1.00 | 54.71 |
| ATOM | 601 | CD1 | LEU | 88 | 29.618 | 25.140 | 76.951 | 1.00 | 54.33 |
| ATOM | 602 | CD2 | LEU | 88 | 29.920 | 25.623 | 79.390 | 1.00 | 54.33 |
| ATOM | 603 | C | LEU | 88 | 30.914 | 21.600 | 77.847 | 1.00 | 52.33 |
| ATOM | 604 | O | LEU | 88 | 31.311 | 21.048 | 78.877 | 1.00 | 53.12 |
| ATOM | 605 | N | VAL | 89 | 31.693 | 21.795 | 76.789 | 1.00 | 52.10 |
| ATOM | 606 | CA | VAL | 89 | 33.078 | 21.342 | 76.788 | 1.00 | 52.46 |
| ATOM | 607 | CB | VAL | 89 | 33.241 | 20.072 | 75.882 | 1.00 | 50.52 |
| ATOM | 608 | CG1 | VAL | 89 | 32.289 | 20.147 | 74.710 | 1.00 | 52.35 |
| ATOM | 609 | CG2 | VAL | 89 | 34.674 | 19.939 | 75.388 | 1.00 | 46.86 |
| ATOM | 610 | C | VAL | 89 | 34.049 | 22.433 | 76.357 | 1.00 | 53.35 |
| ATOM | 611 | O | VAL | 89 | 33.858 | 23.081 | 75.336 | 1.00 | 54.69 |
| ATOM | 612 | N | LYS | 90 | 35.096 | 22.625 | 77.151 | 1.00 | 55.22 |
| ATOM | 613 | CA | LYS | 90 | 36.100 | 23.640 | 76.868 | 1.00 | 56.94 |
| ATOM | 614 | CB | LYS | 90 | 36.656 | 24.205 | 78.181 | 1.00 | 57.66 |
| ATOM | 615 | CG | LYS | 90 | 37.642 | 25.360 | 78.005 | 1.00 | 58.70 |
| ATOM | 616 | CD | LYS | 90 | 38.140 | 25.909 | 79.345 | 1.00 | 59.35 |
| ATOM | 617 | CE | LYS | 90 | 36.995 | 26.399 | 80.226 | 1.00 | 60.64 |
| ATOM | 618 | NZ | LYS | 90 | 36.185 | 27.462 | 79.568 | 1.00 | 61.04 |
| ATOM | 619 | C | LYS | 90 | 37.237 | 23.078 | 76.019 | 1.00 | 57.63 |
| ATOM | 620 | O | LYS | 90 | 37.921 | 22.136 | 76.417 | 1.00 | 57.69 |
| ATOM | 621 | N | VAL | 91 | 37.428 | 23.670 | 74.846 | 1.00 | 58.29 |
| ATOM | 622 | CA | VAL | 91 | 38.473 | 23.254 | 73.919 | 1.00 | 57.11 |
| ATOM | 623 | CB | VAL | 91 | 37.920 | 23.136 | 72.480 | 1.00 | 56.48 |
| ATOM | 624 | CG1 | VAL | 91 | 39.010 | 22.661 | 71.533 | 1.00 | 55.29 |
| ATOM | 625 | CG2 | VAL | 91 | 36.741 | 22.183 | 72.459 | 1.00 | 55.52 |
| ATOM | 626 | C | VAL | 91 | 39.598 | 24.279 | 73.926 | 1.00 | 57.81 |
| ATOM | 627 | O | VAL | 91 | 39.365 | 25.466 | 73.710 | 1.00 | 59.53 |
| ATOM | 628 | N | GLY | 92 | 40.817 | 23.819 | 74.172 | 1.00 | 58.12 |
| ATOM | 629 | CA | GLY | 92 | 41.947 | 24.723 | 74.200 | 1.00 | 59.69 |
| ATOM | 630 | C | GLY | 92 | 43.047 | 24.245 | 73.286 | 1.00 | 61.78 |
| ATOM | 631 | O | GLY | 92 | 42.821 | 23.381 | 72.448 | 1.00 | 61.06 |
| ATOM | 632 | N | GLU | 93 | 44.240 | 24.803 | 73.449 | 1.00 | 65.18 |
| ATOM | 633 | CA | GLU | 93 | 45.373 | 24.426 | 72.619 | 1.00 | 69.00 |
| ATOM | 634 | CB | GLU | 93 | 45.897 | 25.646 | 71.866 | 1.00 | 71.56 |
| ATOM | 635 | CG | GLU | 93 | 47.082 | 25.344 | 70.965 | 1.00 | 75.20 |
| ATOM | 636 | CD | GLU | 93 | 47.659 | 26.591 | 70.325 | 1.00 | 78.28 |
| ATOM | 637 | OE1 | GLU | 93 | 46.893 | 27.326 | 69.659 | 1.00 | 80.05 |
| ATOM | 638 | OE2 | GLU | 93 | 48.877 | 26.834 | 70.485 | 1.00 | 79.21 |
| ATOM | 639 | C | GLU | 93 | 46.505 | 23.822 | 73.437 | 1.00 | 71.00 |
| ATOM | 640 | O | GLU | 93 | 47.118 | 24.500 | 74.263 | 1.00 | 70.74 |
| ATOM | 641 | N | GLY | 94 | 46.784 | 22.544 | 73.195 | 1.00 | 72.97 |
| ATOM | 642 | CA | GLY | 94 | 47.849 | 21.869 | 73.916 | 1.00 | 74.44 |
| ATOM | 643 | C | GLY | 94 | 49.078 | 21.673 | 73.052 | 1.00 | 75.82 |
| ATOM | 644 | O | GLY | 94 | 49.485 | 22.577 | 72.315 | 1.00 | 76.47 |
| ATOM | 645 | N | GLU | 95 | 49.682 | 20.496 | 73.145 | 1.00 | 75.73 |
| ATOM | 646 | CA | GLU | 95 | 50.859 | 20.195 | 72.349 | 1.00 | 76.61 |
| ATOM | 647 | CB | GLU | 95 | 52.023 | 19.792 | 73.249 | 1.00 | 76.93 |
| ATOM | 648 | CG | GLU | 95 | 52.439 | 20.891 | 74.203 | 1.00 | 78.31 |
| ATOM | 649 | CD | GLU | 95 | 53.614 | 20.497 | 75.065 | 1.00 | 78.40 |
| ATOM | 650 | OE1 | GLU | 95 | 54.715 | 20.274 | 74.514 | 1.00 | 78.51 |
| ATOM | 651 | OE2 | GLU | 95 | 53.432 | 20.408 | 76.295 | 1.00 | 78.60 |
| ATOM | 652 | C | GLU | 95 | 50.516 | 19.071 | 71.392 | 1.00 | 76.91 |
| ATOM | 653 | O | GLU | 95 | 49.833 | 18.116 | 71.764 | 1.00 | 76.81 |
| ATOM | 654 | N | GLU | 96 | 50.987 | 19.203 | 70.155 | 1.00 | 77.78 |
| ATOM | 655 | CA | GLU | 96 | 50.733 | 18.220 | 69.105 | 1.00 | 78.07 |
| ATOM | 656 | CB | GLU | 96 | 51.408 | 16.881 | 69.440 | 1.00 | 81.32 |
| ATOM | 657 | CG | GLU | 96 | 52.943 | 16.930 | 69.454 | 1.00 | 85.11 |
| ATOM | 658 | CD | GLU | 96 | 53.541 | 17.309 | 68.101 | 1.00 | 87.05 |
| ATOM | 659 | OE1 | GLU | 96 | 53.346 | 16.551 | 67.124 | 1.00 | 88.73 |
| ATOM | 660 | OE2 | GLU | 96 | 54.207 | 18.365 | 68.014 | 1.00 | 87.56 |
| ATOM | 661 | C | GLU | 96 | 49.230 | 18.025 | 68.919 | 1.00 | 75.88 |
| ATOM | 662 | O | GLU | 96 | 48.784 | 17.039 | 68.327 | 1.00 | 75.92 |
| ATOM | 663 | N | GLY | 97 | 48.456 | 18.980 | 69.427 | 1.00 | 72.88 |
| ATOM | 664 | CA | GLY | 97 | 47.013 | 18.910 | 69.309 | 1.00 | 69.37 |
| ATOM | 665 | C | GLY | 97 | 46.296 | 19.710 | 70.380 | 1.00 | 67.02 |
| ATOM | 666 | O | GLY | 97 | 46.921 | 20.230 | 71.305 | 1.00 | 67.10 |
| ATOM | 667 | N | GLN | 98 | 44.978 | 19.811 | 70.250 | 1.00 | 64.76 |
| ATOM | 668 | CA | GLN | 98 | 44.166 | 20.543 | 71.211 | 1.00 | 62.45 |
| ATOM | 669 | CB | GLN | 98 | 42.872 | 21.045 | 70.562 | 1.00 | 62.69 |
| ATOM | 670 | CG | GLN | 98 | 43.026 | 21.908 | 69.315 | 1.00 | 64.93 |
| ATOM | 671 | CD | GLN | 98 | 43.191 | 21.095 | 68.046 | 1.00 | 65.89 |
| ATOM | 672 | OE1 | GLN | 98 | 44.299 | 20.684 | 67.696 | 1.00 | 65.96 |
| ATOM | 673 | NE2 | GLN | 98 | 42.079 | 20.847 | 67.353 | 1.00 | 65.22 |
| ATOM | 674 | C | GLN | 98 | 43.781 | 19.630 | 72.369 | 1.00 | 61.23 |
| ATOM | 675 | O | GLN | 98 | 43.880 | 18.403 | 72.269 | 1.00 | 62.18 |
| ATOM | 676 | N | TRP | 99 | 43.356 | 20.233 | 73.473 | 1.00 | 57.45 |
| ATOM | 677 | CA | TRP | 99 | 42.893 | 19.459 | 74.611 | 1.00 | 54.44 |
| ATOM | 678 | CB | TRP | 99 | 43.639 | 19.822 | 75.904 | 1.00 | 55.51 |
| ATOM | 679 | CG | TRP | 99 | 43.770 | 21.291 | 76.211 | 1.00 | 56.94 |
| ATOM | 680 | CD2 | TRP | 99 | 42.763 | 22.151 | 76.756 | 1.00 | 56.03 |
| ATOM | 681 | CE2 | TRP | 99 | 43.345 | 23.426 | 76.922 | 1.00 | 57.25 |
| ATOM | 682 | CE3 | TRP | 99 | 41.422 | 21.969 | 77.121 | 1.00 | 56.67 |
| ATOM | 683 | CD1 | TRP | 99 | 44.892 | 22.062 | 76.068 | 1.00 | 56.29 |
| ATOM | 684 | NE1 | TRP | 99 | 44.647 | 23.342 | 76.495 | 1.00 | 56.55 |
| ATOM | 685 | CZ2 | TRP | 99 | 42.635 | 24.516 | 77.440 | 1.00 | 56.53 |
| ATOM | 686 | CZ3 | TRP | 99 | 40.712 | 23.053 | 77.637 | 1.00 | 56.67 |
| ATOM | 687 | CH2 | TRP | 99 | 41.322 | 24.309 | 77.790 | 1.00 | 56.70 |
| ATOM | 688 | C | TRP | 99 | 41.408 | 19.756 | 74.737 | 1.00 | 52.04 |
| ATOM | 689 | O | TRP | 99 | 40.899 | 20.664 | 74.089 | 1.00 | 50.70 |
| ATOM | 690 | N | SER | 100 | 40.704 | 18.981 | 75.545 | 1.00 | 49.57 |
| ATOM | 691 | CA | SER | 100 | 39.277 | 19.186 | 75.715 | 1.00 | 48.29 |
| ATOM | 692 | CB | SER | 100 | 38.506 | 18.475 | 74.597 | 1.00 | 49.26 |
| ATOM | 693 | OG | SER | 100 | 39.055 | 17.196 | 74.315 | 1.00 | 47.27 |
| ATOM | 694 | C | SER | 100 | 38.860 | 18.655 | 77.067 | 1.00 | 47.91 |
| ATOM | 695 | O | SER | 100 | 39.569 | 17.845 | 77.662 | 1.00 | 48.73 |
| ATOM | 696 | N | VAL | 101 | 37.718 | 19.120 | 77.558 | 1.00 | 47.53 |
| ATOM | 697 | CA | VAL | 101 | 37.225 | 18.684 | 78.852 | 1.00 | 47.86 |
| ATOM | 698 | CB | VAL | 101 | 38.102 | 19.233 | 79.995 | 1.00 | 47.92 |
| ATOM | 699 | CG1 | VAL | 101 | 38.160 | 20.747 | 79.923 | 1.00 | 49.02 |
| ATOM | 700 | CG2 | VAL | 101 | 37.545 | 18.783 | 81.342 | 1.00 | 47.98 |
| ATOM | 701 | C | VAL | 101 | 35.784 | 19.102 | 79.101 | 1.00 | 48.77 |
| ATOM | 702 | O | VAL | 101 | 35.391 | 20.228 | 78.798 | 1.00 | 49.05 |
| ATOM | 703 | N | LYS | 102 | 35.004 | 18.176 | 79.649 | 1.00 | 49.04 |
| ATOM | 704 | CA | LYS | 102 | 33.607 | 18.422 | 79.969 | 1.00 | 50.31 |
| ATOM | 705 | CB | LYS | 102 | 32.875 | 17.101 | 80.220 | 1.00 | 51.15 |
| ATOM | 706 | CG | LYS | 102 | 31.385 | 17.263 | 80.452 | 1.00 | 52.57 |
| ATOM | 707 | CD | LYS | 102 | 30.835 | 16.229 | 81.425 | 1.00 | 56.56 |
| ATOM | 708 | CE | LYS | 102 | 30.955 | 14.804 | 80.908 | 1.00 | 57.06 |
| ATOM | 709 | NZ | LYS | 102 | 30.275 | 13.804 | 81.787 | 1.00 | 58.08 |
| ATOM | 710 | C | LYS | 102 | 33.587 | 19.254 | 81.243 | 1.00 | 51.12 |
| ATOM | 711 | O | LYS | 102 | 34.220 | 18.888 | 82.234 | 1.00 | 52.47 |
| ATOM | 712 | N | THR | 103 | 32.859 | 20.366 | 81.217 | 1.00 | 51.40 |
| ATOM | 713 | CA | THR | 103 | 32.774 | 21.252 | 82.373 | 1.00 | 50.47 |
| ATOM | 714 | CB | THR | 103 | 33.004 | 22.715 | 81.965 | 1.00 | 50.28 |
| ATOM | 715 | OG1 | THR | 103 | 31.992 | 23.113 | 81.032 | 1.00 | 51.29 |
| ATOM | 716 | CG2 | THR | 103 | 34.368 | 22.879 | 81.324 | 1.00 | 47.52 |
| ATOM | 717 | C | THR | 103 | 31.416 | 21.148 | 83.048 | 1.00 | 50.90 |
| ATOM | 718 | O | THR | 103 | 31.329 | 21.056 | 84.268 | 1.00 | 50.91 |
| ATOM | 719 | N | LYS | 104 | 30.358 | 21.162 | 82.247 | 1.00 | 52.41 |
| ATOM | 720 | CA | LYS | 104 | 29.000 | 21.063 | 82.770 | 1.00 | 54.04 |
| ATOM | 721 | CB | LYS | 104 | 28.310 | 22.436 | 82.714 | 1.00 | 57.21 |
| ATOM | 722 | CG | LYS | 104 | 28.823 | 23.450 | 83.739 | 1.00 | 59.16 |
| ATOM | 723 | CD | LYS | 104 | 28.138 | 24.809 | 83.576 | 1.00 | 62.54 |
| ATOM | 724 | CE | LYS | 104 | 28.398 | 25.734 | 84.766 | 1.00 | 62.99 |
| ATOM | 725 | NZ | LYS | 104 | 27.798 | 25.217 | 86.037 | 1.00 | 64.17 |
| ATOM | 726 | C | LYS | 104 | 28.215 | 20.047 | 81.948 | 1.00 | 53.79 |
| ATOM | 727 | O | LYS | 104 | 28.411 | 19.941 | 80.740 | 1.00 | 53.53 |
| ATOM | 728 | N | HIS | 105 | 27.330 | 19.299 | 82.600 | 1.00 | 53.65 |
| ATOM | 729 | CA | HIS | 105 | 26.539 | 18.295 | 81.903 | 1.00 | 55.05 |
| ATOM | 730 | CB | HIS | 105 | 27.316 | 16.972 | 81.837 | 1.00 | 55.94 |
| ATOM | 731 | CG | HIS | 105 | 27.668 | 16.397 | 83.176 | 1.00 | 55.84 |
| ATOM | 732 | CD2 | HIS | 105 | 28.793 | 16.501 | 83.924 | 1.00 | 55.19 |
| ATOM | 733 | ND1 | HIS | 105 | 26.803 | 15.602 | 83.897 | 1.00 | 55.83 |
| ATOM | 734 | CE1 | HIS | 105 | 27.380 | 15.241 | 85.030 | 1.00 | 56.35 |
| ATOM | 735 | NE2 | HIS | 105 | 28.589 | 15.773 | 85.071 | 1.00 | 55.64 |
| ATOM | 736 | C | HIS | 105 | 25.169 | 18.074 | 82.534 | 1.00 | 56.32 |
| ATOM | 737 | O | HIS | 105 | 24.903 | 18.535 | 83.640 | 1.00 | 56.55 |
| ATOM | 738 | N | GLN | 106 | 24.302 | 17.365 | 81.817 | 1.00 | 58.21 |
| ATOM | 739 | CA | GLN | 106 | 22.950 | 17.090 | 82.289 | 1.00 | 60.74 |
| ATOM | 740 | CB | GLN | 106 | 22.108 | 18.367 | 82.224 | 1.00 | 61.97 |
| ATOM | 741 | CG | GLN | 106 | 20.775 | 18.285 | 82.945 | 1.00 | 64.86 |
| ATOM | 742 | CD | GLN | 106 | 20.928 | 18.379 | 84.447 | 1.00 | 67.03 |
| ATOM | 743 | OE1 | GLN | 106 | 21.447 | 19.370 | 84.969 | 1.00 | 68.82 |
| ATOM | 744 | NE2 | GLN | 106 | 20.479 | 17.348 | 85.155 | 1.00 | 67.41 |
| ATOM | 745 | C | GLN | 106 | 22.322 | 16.025 | 81.396 | 1.00 | 61.62 |
| ATOM | 746 | O | GLN | 106 | 22.532 | 16.027 | 80.186 | 1.00 | 62.03 |
| ATOM | 747 | N | MET | 107 | 21.550 | 15.121 | 81.990 | 1.00 | 63.03 |
| ATOM | 748 | CA | MET | 107 | 20.900 | 14.058 | 81.232 | 1.00 | 64.74 |
| ATOM | 749 | CB | MET | 107 | 21.322 | 12.688 | 81.769 | 1.00 | 66.23 |
| ATOM | 750 | CG | MET | 107 | 22.821 | 12.456 | 81.786 | 1.00 | 68.74 |
| ATOM | 751 | SD | MET | 107 | 23.248 | 10.812 | 82.388 | 1.00 | 70.84 |
| ATOM | 752 | CE | MET | 107 | 23.427 | 9.926 | 80.853 | 1.00 | 71.13 |
| ATOM | 753 | C | MET | 107 | 19.385 | 14.175 | 81.313 | 1.00 | 65.81 |
| ATOM | 754 | O | MET | 107 | 18.837 | 14.489 | 82.369 | 1.00 | 65.52 |
| ATOM | 755 | N | TYR | 108 | 18.712 | 13.915 | 80.196 | 1.00 | 66.87 |
| ATOM | 756 | CA | TYR | 108 | 17.258 | 13.984 | 80.143 | 1.00 | 68.20 |
| ATOM | 757 | CB | TYR | 108 | 16.800 | 15.167 | 79.286 | 1.00 | 67.20 |
| ATOM | 758 | CG | TYR | 108 | 17.436 | 16.484 | 79.660 | 1.00 | 66.35 |
| ATOM | 759 | CD1 | TYR | 108 | 18.781 | 16.731 | 79.386 | 1.00 | 65.95 |
| ATOM | 760 | CE1 | TYR | 108 | 19.380 | 17.929 | 79.746 | 1.00 | 65.76 |
| ATOM | 761 | CD2 | TYR | 108 | 16.702 | 17.477 | 80.307 | 1.00 | 66.24 |
| ATOM | 762 | CE2 | TYR | 108 | 17.292 | 18.683 | 80.674 | 1.00 | 65.93 |
| ATOM | 763 | CZ | TYR | 108 | 18.633 | 18.902 | 80.391 | 1.00 | 66.14 |
| ATOM | 764 | OH | TYR | 108 | 19.235 | 20.083 | 80.763 | 1.00 | 64.27 |
| ATOM | 765 | C | TYR | 108 | 16.706 | 12.700 | 79.549 | 1.00 | 70.20 |
| ATOM | 766 | O | TYR | 108 | 16.995 | 12.363 | 78.404 | 1.00 | 70.55 |
| ATOM | 767 | N | SER | 109 | 15.912 | 11.982 | 80.331 | 1.00 | 73.54 |
| ATOM | 768 | CA | SER | 109 | 15.322 | 10.739 | 79.863 | 1.00 | 76.84 |
| ATOM | 769 | CB | SER | 109 | 14.524 | 10.082 | 80.992 | 1.00 | 77.63 |
| ATOM | 770 | OG | SER | 109 | 15.353 | 9.837 | 82.120 | 1.00 | 78.13 |
| ATOM | 771 | C | SER | 109 | 14.419 | 11.020 | 78.664 | 1.00 | 78.98 |
| ATOM | 772 | O | SER | 109 | 13.936 | 12.138 | 78.486 | 1.00 | 78.51 |
| ATOM | 773 | N | ILE | 110 | 14.198 | 10.002 | 77.841 | 1.00 | 82.34 |
| ATOM | 774 | CA | ILE | 110 | 13.369 | 10.143 | 76.651 | 1.00 | 86.07 |
| ATOM | 775 | CB | ILE | 110 | 13.892 | 9.249 | 75.511 | 1.00 | 86.28 |
| ATOM | 776 | CG2 | ILE | 110 | 13.092 | 9.505 | 74.242 | 1.00 | 86.56 |
| ATOM | 777 | CG1 | ILE | 110 | 15.379 | 9.529 | 75.275 | 1.00 | 86.19 |
| ATOM | 778 | CD1 | ILE | 110 | 16.025 | 8.612 | 74.258 | 1.00 | 86.76 |
| ATOM | 779 | C | ILE | 110 | 11.916 | 9.772 | 76.927 | 1.00 | 88.58 |
| ATOM | 780 | O | ILE | 110 | 11.596 | 8.606 | 77.152 | 1.00 | 88.69 |
| ATOM | 781 | N | PRO | 111 | 11.016 | 10.767 | 76.910 | 1.00 | 91.13 |
| ATOM | 782 | CD | PRO | 111 | 11.319 | 12.205 | 76.811 | 1.00 | 91.83 |
| ATOM | 783 | CA | PRO | 111 | 9.585 | 10.562 | 77.157 | 1.00 | 93.32 |
| ATOM | 784 | CB | PRO | 111 | 9.015 | 11.975 | 77.062 | 1.00 | 93.16 |
| ATOM | 785 | CG | PRO | 111 | 10.147 | 12.819 | 77.536 | 1.00 | 92.31 |
| ATOM | 786 | C | PRO | 111 | 8.928 | 9.613 | 76.159 | 1.00 | 95.40 |
| ATOM | 787 | O | PRO | 111 | 9.466 | 9.355 | 75.082 | 1.00 | 95.80 |
| ATOM | 788 | N | GLU | 112 | 7.758 | 9.101 | 76.529 | 1.00 | 97.55 |
| ATOM | 789 | CA | GLU | 112 | 7.006 | 8.185 | 75.679 | 1.00 | 99.50 |
| ATOM | 790 | CB | GLU | 112 | 5.816 | 7.611 | 76.458 | 1.00 | 100.31 |
| ATOM | 791 | CG | GLU | 112 | 4.745 | 6.971 | 75.589 | 1.00 | 101.76 |
| ATOM | 792 | CD | GLU | 112 | 5.316 | 5.989 | 74.587 | 1.00 | 102.84 |
| ATOM | 793 | OE1 | GLU | 112 | 5.967 | 5.012 | 75.014 | 1.00 | 103.66 |
| ATOM | 794 | OE2 | GLU | 112 | 5.113 | 6.196 | 73.372 | 1.00 | 103.00 |
| ATOM | 795 | C | GLU | 112 | 6.508 | 8.884 | 74.418 | 1.00 | 100.37 |
| ATOM | 796 | O | GLU | 112 | 6.914 | 8.545 | 73.304 | 1.00 | 100.17 |
| ATOM | 797 | N | ASP | 113 | 5.625 | 9.859 | 74.606 | 1.00 | 101.44 |
| ATOM | 798 | CA | ASP | 113 | 5.056 | 10.620 | 73.499 | 1.00 | 102.05 |
| ATOM | 799 | CB | ASP | 113 | 4.087 | 11.680 | 74.038 | 1.00 | 102.23 |
| ATOM | 800 | CG | ASP | 113 | 4.682 | 12.494 | 75.177 | 1.00 | 102.33 |
| ATOM | 801 | OD1 | ASP | 113 | 4.961 | 11.913 | 76.249 | 1.00 | 102.01 |
| ATOM | 802 | OD2 | ASP | 113 | 4.870 | 13.716 | 74.999 | 1.00 | 101.99 |
| ATOM | 803 | C | ASP | 113 | 6.131 | 11.282 | 72.638 | 1.00 | 102.09 |
| ATOM | 804 | O | ASP | 113 | 5.843 | 11.789 | 71.553 | 1.00 | 101.96 |
| ATOM | 805 | N | ALA | 114 | 7.368 | 11.273 | 73.126 | 1.00 | 102.12 |
| ATOM | 806 | CA | ALA | 114 | 8.484 | 11.869 | 72.401 | 1.00 | 102.09 |
| ATOM | 807 | CB | ALA | 114 | 9.590 | 12.256 | 73.377 | 1.00 | 101.76 |
| ATOM | 808 | C | ALA | 114 | 9.022 | 10.895 | 71.358 | 1.00 | 102.06 |
| ATOM | 809 | O | ALA | 114 | 9.763 | 11.282 | 70.455 | 1.00 | 101.89 |
| ATOM | 810 | N | MET | 115 | 8.640 | 9.630 | 71.491 | 1.00 | 102.04 |
| ATOM | 811 | CA | MET | 115 | 9.081 | 8.592 | 70.569 | 1.00 | 102.05 |
| ATOM | 812 | CB | MET | 115 | 9.466 | 7.331 | 71.346 | 1.00 | 102.77 |
| ATOM | 813 | CG | MET | 115 | 10.637 | 7.509 | 72.307 | 1.00 | 103.47 |
| ATOM | 814 | SD | MET | 115 | 12.256 | 7.549 | 71.502 | 1.00 | 104.26 |
| ATOM | 815 | CE | MET | 115 | 12.740 | 5.824 | 71.638 | 1.00 | 103.48 |
| ATOM | 816 | C | MET | 115 | 8.004 | 8.253 | 69.538 | 1.00 | 101.77 |
| ATOM | 817 | O | MET | 115 | 8.268 | 8.275 | 68.337 | 1.00 | 102.14 |
| ATOM | 818 | N | THR | 116 | 6.796 | 7.942 | 70.006 | 1.00 | 101.14 |
| ATOM | 819 | CA | THR | 116 | 5.690 | 7.590 | 69.110 | 1.00 | 100.36 |
| ATOM | 820 | CB | THR | 116 | 4.517 | 6.927 | 69.880 | 1.00 | 100.42 |
| ATOM | 821 | OG1 | THR | 116 | 5.004 | 5.805 | 70.625 | 1.00 | 100.29 |
| ATOM | 822 | CG2 | THR | 116 | 3.441 | 6.441 | 68.911 | 1.00 | 100.05 |
| ATOM | 823 | C | THR | 116 | 5.150 | 8.816 | 68.379 | 1.00 | 99.62 |
| ATOM | 824 | O | THR | 116 | 4.423 | 8.694 | 67.391 | 1.00 | 99.72 |
| ATOM | 825 | N | GLY | 117 | 5.510 | 9.996 | 68.870 | 1.00 | 98.62 |
| ATOM | 826 | CA | GLY | 117 | 5.048 | 11.224 | 68.252 | 1.00 | 97.42 |
| ATOM | 827 | C | GLY | 117 | 5.619 | 11.447 | 66.866 | 1.00 | 96.48 |
| ATOM | 828 | O | GLY | 117 | 5.746 | 10.511 | 66.074 | 1.00 | 96.38 |
| ATOM | 829 | N | THR | 118 | 5.962 | 12.696 | 66.570 | 1.00 | 95.25 |
| ATOM | 830 | CA | THR | 118 | 6.521 | 13.050 | 65.273 | 1.00 | 93.78 |
| ATOM | 831 | CB | THR | 118 | 5.679 | 14.133 | 64.578 | 1.00 | 93.57 |
| ATOM | 832 | OG1 | THR | 118 | 5.735 | 15.343 | 65.342 | 1.00 | 93.50 |
| ATOM | 833 | CG2 | THR | 118 | 4.234 | 13.685 | 64.457 | 1.00 | 93.65 |
| ATOM | 834 | C | THR | 118 | 7.936 | 13.583 | 65.440 | 1.00 | 92.67 |
| ATOM | 835 | O | THR | 118 | 8.335 | 13.976 | 66.537 | 1.00 | 92.39 |
| ATOM | 836 | N | ALA | 119 | 8.687 | 13.593 | 64.343 | 1.00 | 91.30 |
| ATOM | 837 | CA | ALA | 119 | 10.058 | 14.084 | 64.356 | 1.00 | 90.00 |
| ATOM | 838 | CB | ALA | 119 | 10.643 | 14.031 | 62.956 | 1.00 | 89.81 |
| ATOM | 839 | C | ALA | 119 | 10.066 | 15.513 | 64.867 | 1.00 | 89.21 |
| ATOM | 840 | O | ALA | 119 | 11.045 | 15.972 | 65.455 | 1.00 | 88.98 |
| ATOM | 841 | N | GLU | 120 | 8.959 | 16.210 | 64.636 | 1.00 | 88.61 |
| ATOM | 842 | CA | GLU | 120 | 8.819 | 17.593 | 65.063 | 1.00 | 87.61 |
| ATOM | 843 | CB | GLU | 120 | 7.505 | 18.177 | 64.536 | 1.00 | 87.74 |
| ATOM | 844 | CG | GLU | 120 | 7.138 | 17.763 | 63.112 | 1.00 | 86.31 |
| ATOM | 845 | CD | GLU | 120 | 8.269 | 17.956 | 62.120 | 1.00 | 85.84 |
| ATOM | 846 | OE1 | GLU | 120 | 8.884 | 19.042 | 62.113 | 1.00 | 84.76 |
| ATOM | 847 | OE2 | GLU | 120 | 8.535 | 17.020 | 61.336 | 1.00 | 85.71 |
| ATOM | 848 | C | GLU | 120 | 8.837 | 17.658 | 66.588 | 1.00 | 86.71 |
| ATOM | 849 | O | GLU | 120 | 9.610 | 18.412 | 67.179 | 1.00 | 86.71 |
| ATOM | 850 | N | MET | 121 | 7.980 | 16.859 | 67.216 | 1.00 | 85.74 |
| ATOM | 851 | CA | MET | 121 | 7.895 | 16.817 | 68.671 | 1.00 | 84.85 |
| ATOM | 852 | CB | MET | 121 | 6.798 | 15.842 | 69.111 | 1.00 | 84.04 |
| ATOM | 853 | CG | MET | 121 | 5.390 | 16.273 | 68.740 | 1.00 | 81.88 |
| ATOM | 854 | SD | MET | 121 | 4.152 | 15.078 | 69.268 | 1.00 | 80.83 |
| ATOM | 855 | CE | MET | 121 | 3.772 | 14.283 | 67.730 | 1.00 | 78.55 |
| ATOM | 856 | C | MET | 121 | 9.226 | 16.397 | 69.286 | 1.00 | 84.73 |
| ATOM | 857 | O | MET | 121 | 9.687 | 17.003 | 70.255 | 1.00 | 84.87 |
| ATOM | 858 | N | LEU | 122 | 9.839 | 15.360 | 68.717 | 1.00 | 84.21 |
| ATOM | 859 | CA | LEU | 122 | 11.115 | 14.851 | 69.211 | 1.00 | 83.20 |
| ATOM | 860 | CB | LEU | 122 | 11.711 | 13.847 | 68.221 | 1.00 | 83.29 |
| ATOM | 861 | CG | LEU | 122 | 12.966 | 13.109 | 68.697 | 1.00 | 83.07 |
| ATOM | 862 | CD1 | LEU | 122 | 12.612 | 12.232 | 69.885 | 1.00 | 82.78 |
| ATOM | 863 | CD2 | LEU | 122 | 13.533 | 12.261 | 67.572 | 1.00 | 82.52 |
| ATOM | 864 | C | LEU | 122 | 12.110 | 15.980 | 69.448 | 1.00 | 82.61 |
| ATOM | 865 | O | LEU | 122 | 12.546 | 16.204 | 70.575 | 1.00 | 82.47 |
| ATOM | 866 | N | PHE | 123 | 12.467 | 16.694 | 68.385 | 1.00 | 82.28 |
| ATOM | 867 | CA | PHE | 123 | 13.414 | 17.794 | 68.512 | 1.00 | 82.09 |
| ATOM | 868 | CB | PHE | 123 | 13.898 | 18.251 | 67.136 | 1.00 | 82.08 |
| ATOM | 869 | CG | PHE | 123 | 14.948 | 17.357 | 66.547 | 1.00 | 81.61 |
| ATOM | 870 | CD1 | PHE | 123 | 14.616 | 16.098 | 66.060 | 1.00 | 81.34 |
| ATOM | 871 | CD2 | PHE | 123 | 16.281 | 17.756 | 66.523 | 1.00 | 81.33 |
| ATOM | 872 | CE1 | PHE | 123 | 15.594 | 15.246 | 65.559 | 1.00 | 80.67 |
| ATOM | 873 | CE2 | PHE | 123 | 17.268 | 16.912 | 66.026 | 1.00 | 81.58 |
| ATOM | 874 | CZ | PHE | 123 | 16.923 | 15.653 | 65.543 | 1.00 | 81.33 |
| ATOM | 875 | C | PHE | 123 | 12.834 | 18.964 | 69.288 | 1.00 | 81.98 |
| ATOM | 876 | O | PHE | 123 | 13.570 | 19.838 | 69.747 | 1.00 | 81.74 |
| ATOM | 877 | N | ASP | 124 | 11.512 | 18.980 | 69.429 | 1.00 | 82.09 |
| ATOM | 878 | CA | ASP | 124 | 10.852 | 20.028 | 70.195 | 1.00 | 82.29 |
| ATOM | 879 | CB | ASP | 124 | 9.329 | 19.909 | 70.073 | 1.00 | 81.96 |
| ATOM | 880 | CG | ASP | 124 | 8.731 | 20.961 | 69.157 | 1.00 | 81.56 |
| ATOM | 881 | OD1 | ASP | 124 | 7.510 | 20.897 | 68.901 | 1.00 | 81.25 |
| ATOM | 882 | OD2 | ASP | 124 | 9.477 | 21.855 | 68.701 | 1.00 | 80.94 |
| ATOM | 883 | C | ASP | 124 | 11.279 | 19.808 | 71.641 | 1.00 | 82.22 |
| ATOM | 884 | O | ASP | 124 | 11.819 | 20.707 | 72.287 | 1.00 | 81.61 |
| ATOM | 885 | N | TYR | 125 | 11.047 | 18.595 | 72.133 | 1.00 | 82.59 |
| ATOM | 886 | CA | TYR | 125 | 11.420 | 18.233 | 73.494 | 1.00 | 83.66 |
| ATOM | 887 | CB | TYR | 125 | 11.048 | 16.771 | 73.767 | 1.00 | 85.84 |
| ATOM | 888 | CG | TYR | 125 | 11.533 | 16.240 | 75.100 | 1.00 | 88.74 |
| ATOM | 889 | CD1 | TYR | 125 | 12.763 | 15.590 | 75.209 | 1.00 | 89.83 |
| ATOM | 890 | CE1 | TYR | 125 | 13.222 | 15.110 | 76.437 | 1.00 | 91.28 |
| ATOM | 891 | CD2 | TYR | 125 | 10.770 | 16.399 | 76.257 | 1.00 | 90.41 |
| ATOM | 892 | CE2 | TYR | 125 | 11.221 | 15.926 | 77.493 | 1.00 | 91.86 |
| ATOM | 893 | CZ | TYR | 125 | 12.448 | 15.281 | 77.574 | 1.00 | 92.09 |
| ATOM | 894 | OH | TYR | 125 | 12.896 | 14.807 | 78.789 | 1.00 | 93.08 |
| ATOM | 895 | C | TYR | 125 | 12.917 | 18.451 | 73.704 | 1.00 | 82.86 |
| ATOM | 896 | O | TYR | 125 | 13.352 | 18.829 | 74.792 | 1.00 | 82.74 |
| ATOM | 897 | N | ILE | 126 | 13.701 | 18.215 | 72.655 | 1.00 | 81.74 |
| ATOM | 898 | CA | ILE | 126 | 15.146 | 18.398 | 72.727 | 1.00 | 80.58 |
| ATOM | 899 | CB | ILE | 126 | 15.824 | 18.005 | 71.397 | 1.00 | 79.32 |
| ATOM | 900 | CG2 | ILE | 126 | 17.277 | 18.443 | 71.398 | 1.00 | 78.57 |
| ATOM | 901 | CG1 | ILE | 126 | 15.719 | 16.494 | 71.194 | 1.00 | 78.47 |
| ATOM | 902 | CD1 | ILE | 126 | 16.408 | 15.993 | 69.946 | 1.00 | 78.42 |
| ATOM | 903 | C | ILE | 126 | 15.479 | 19.852 | 73.047 | 1.00 | 80.87 |
| ATOM | 904 | O | ILE | 126 | 16.334 | 20.133 | 73.887 | 1.00 | 79.71 |
| ATOM | 905 | N | SER | 127 | 14.799 | 20.772 | 72.370 | 1.00 | 81.80 |
| ATOM | 906 | CA | SER | 127 | 15.018 | 22.196 | 72.594 | 1.00 | 82.44 |
| ATOM | 907 | CB | SER | 127 | 14.160 | 23.021 | 71.636 | 1.00 | 82.62 |
| ATOM | 908 | OG | SER | 127 | 14.559 | 22.807 | 70.294 | 1.00 | 83.20 |
| ATOM | 909 | C | SER | 127 | 14.668 | 22.543 | 74.034 | 1.00 | 82.44 |
| ATOM | 910 | O | SER | 127 | 15.318 | 23.382 | 74.660 | 1.00 | 81.86 |
| ATOM | 911 | N | GLU | 128 | 13.636 | 21.884 | 74.553 | 1.00 | 83.04 |
| ATOM | 912 | CA | GLU | 128 | 13.202 | 22.106 | 75.927 | 1.00 | 83.79 |
| ATOM | 913 | CB | GLU | 128 | 11.944 | 21.289 | 76.232 | 1.00 | 84.79 |
| ATOM | 914 | CG | GLU | 128 | 11.408 | 21.490 | 77.645 | 1.00 | 86.70 |
| ATOM | 915 | CD | GLU | 128 | 10.425 | 20.409 | 78.061 | 1.00 | 88.14 |
| ATOM | 916 | OE1 | GLU | 128 | 9.408 | 20.222 | 77.357 | 1.00 | 88.36 |
| ATOM | 917 | OE2 | GLU | 128 | 10.672 | 19.747 | 79.094 | 1.00 | 88.06 |
| ATOM | 918 | C | GLU | 128 | 14.318 | 21.686 | 76.877 | 1.00 | 83.42 |
| ATOM | 919 | O | GLU | 128 | 14.483 | 22.261 | 77.952 | 1.00 | 84.16 |
| ATOM | 920 | N | CYS | 129 | 15.081 | 20.675 | 76.475 | 1.00 | 82.77 |
| ATOM | 921 | CA | CYS | 129 | 16.177 | 20.179 | 77.295 | 1.00 | 81.21 |
| ATOM | 922 | CB | CYS | 129 | 16.554 | 18.760 | 76.873 | 1.00 | 81.07 |
| ATOM | 923 | SG | CYS | 129 | 15.206 | 17.569 | 77.006 | 1.00 | 80.63 |
| ATOM | 924 | C | CYS | 129 | 17.391 | 21.089 | 77.178 | 1.00 | 80.64 |
| ATOM | 925 | O | CYS | 129 | 18.092 | 21.330 | 78.160 | 1.00 | 79.84 |
| ATOM | 926 | N | ILE | 130 | 17.644 | 21.591 | 75.975 | 1.00 | 80.16 |
| ATOM | 927 | CA | ILE | 130 | 18.782 | 22.475 | 75.775 | 1.00 | 80.33 |
| ATOM | 928 | CB | ILE | 130 | 18.944 | 22.860 | 74.298 | 1.00 | 79.59 |
| ATOM | 929 | CG2 | ILE | 130 | 20.253 | 23.614 | 74.102 | 1.00 | 79.29 |
| ATOM | 930 | CG1 | ILE | 130 | 18.933 | 21.599 | 73.436 | 1.00 | 79.00 |
| ATOM | 931 | CD1 | ILE | 130 | 19.069 | 21.860 | 71.958 | 1.00 | 79.73 |
| ATOM | 932 | C | ILE | 130 | 18.559 | 23.735 | 76.595 | 1.00 | 80.49 |
| ATOM | 933 | O | ILE | 130 | 19.475 | 24.241 | 77.239 | 1.00 | 80.22 |
| ATOM | 934 | N | SER | 131 | 17.326 | 24.229 | 76.574 | 1.00 | 81.09 |
| ATOM | 935 | CA | SER | 131 | 16.970 | 25.428 | 77.320 | 1.00 | 82.28 |
| ATOM | 936 | CB | SER | 131 | 15.525 | 25.826 | 77.006 | 1.00 | 83.15 |
| ATOM | 937 | OG | SER | 131 | 14.641 | 24.736 | 77.195 | 1.00 | 82.88 |
| ATOM | 938 | C | SER | 131 | 17.136 | 25.195 | 78.820 | 1.00 | 82.33 |
| ATOM | 939 | O | SER | 131 | 17.843 | 25.940 | 79.501 | 1.00 | 82.07 |
| ATOM | 940 | N | ASP | 132 | 16.478 | 24.155 | 79.322 | 1.00 | 82.42 |
| ATOM | 941 | CA | ASP | 132 | 16.540 | 23.792 | 80.735 | 1.00 | 82.24 |
| ATOM | 942 | CB | ASP | 132 | 15.893 | 22.411 | 80.934 | 1.00 | 83.24 |
| ATOM | 943 | CG | ASP | 132 | 15.836 | 21.981 | 82.393 | 1.00 | 83.66 |
| ATOM | 944 | OD1 | ASP | 132 | 15.165 | 20.963 | 82.678 | 1.00 | 83.28 |
| ATOM | 945 | OD2 | ASP | 132 | 16.458 | 22.645 | 83.250 | 1.00 | 83.85 |
| ATOM | 946 | C | ASP | 132 | 17.996 | 23.778 | 81.200 | 1.00 | 81.62 |
| ATOM | 947 | O | ASP | 132 | 18.324 | 24.293 | 82.270 | 1.00 | 82.12 |
| ATOM | 948 | N | PHE | 133 | 18.866 | 23.193 | 80.383 | 1.00 | 80.65 |
| ATOM | 949 | CA | PHE | 133 | 20.286 | 23.118 | 80.698 | 1.00 | 79.47 |
| ATOM | 950 | CB | PHE | 133 | 21.033 | 22.331 | 79.616 | 1.00 | 77.80 |
| ATOM | 951 | CG | PHE | 133 | 22.528 | 22.391 | 79.750 | 1.00 | 75.86 |
| ATOM | 952 | CD1 | PHE | 133 | 23.178 | 21.695 | 80.761 | 1.00 | 75.50 |
| ATOM | 953 | CD2 | PHE | 133 | 23.284 | 23.179 | 78.889 | 1.00 | 75.39 |
| ATOM | 954 | CE1 | PHE | 133 | 24.562 | 21.785 | 80.914 | 1.00 | 74.78 |
| ATOM | 955 | CE2 | PHE | 133 | 24.667 | 23.275 | 79.035 | 1.00 | 74.59 |
| ATOM | 956 | CZ | PHE | 133 | 25.305 | 22.578 | 80.049 | 1.00 | 74.18 |
| ATOM | 957 | C | PHE | 133 | 20.876 | 24.519 | 80.786 | 1.00 | 79.33 |
| ATOM | 958 | O | PHE | 133 | 21.690 | 24.810 | 81.659 | 1.00 | 79.06 |
| ATOM | 959 | N | LEU | 134 | 20.459 | 25.382 | 79.869 | 1.00 | 79.23 |
| ATOM | 960 | CA | LEU | 134 | 20.951 | 26.748 | 79.828 | 1.00 | 79.59 |
| ATOM | 961 | CB | LEU | 134 | 20.482 | 27.412 | 78.534 | 1.00 | 79.43 |
| ATOM | 962 | CG | LEU | 134 | 21.043 | 26.703 | 77.297 | 1.00 | 78.61 |
| ATOM | 963 | CD1 | LEU | 134 | 20.401 | 27.247 | 76.032 | 1.00 | 78.47 |
| ATOM | 964 | CD2 | LEU | 134 | 22.554 | 26.878 | 77.264 | 1.00 | 77.75 |
| ATOM | 965 | C | LEU | 134 | 20.524 | 27.565 | 81.043 | 1.00 | 79.41 |
| ATOM | 966 | O | LEU | 134 | 21.324 | 28.310 | 81.609 | 1.00 | 78.74 |
| ATOM | 967 | N | ASP | 135 | 19.268 | 27.423 | 81.448 | 1.00 | 80.16 |
| ATOM | 968 | CA | ASP | 135 | 18.780 | 28.152 | 82.609 | 1.00 | 80.92 |
| ATOM | 969 | CB | ASP | 135 | 17.271 | 27.966 | 82.777 | 1.00 | 80.81 |
| ATOM | 970 | CG | ASP | 135 | 16.474 | 28.778 | 81.783 | 1.00 | 81.08 |
| ATOM | 971 | OD1 | ASP | 135 | 16.801 | 29.970 | 81.599 | 1.00 | 82.67 |
| ATOM | 972 | OD2 | ASP | 135 | 15.517 | 28.234 | 81.195 | 1.00 | 81.12 |
| ATOM | 973 | C | ASP | 135 | 19.486 | 27.686 | 83.872 | 1.00 | 81.80 |
| ATOM | 974 | O | ASP | 135 | 20.090 | 28.490 | 84.578 | 1.00 | 82.12 |
| ATOM | 975 | N | LYS | 136 | 19.418 | 26.384 | 84.143 | 1.00 | 82.43 |
| ATOM | 976 | CA | LYS | 136 | 20.041 | 25.811 | 85.333 | 1.00 | 83.25 |
| ATOM | 977 | CB | LYS | 136 | 19.750 | 24.307 | 85.418 | 1.00 | 82.64 |
| ATOM | 978 | CG | LYS | 136 | 18.288 | 23.970 | 85.677 | 1.00 | 82.57 |
| ATOM | 979 | CD | LYS | 136 | 18.095 | 22.487 | 85.952 | 1.00 | 82.49 |
| ATOM | 980 | CE | LYS | 136 | 16.630 | 22.154 | 86.182 | 1.00 | 82.31 |
| ATOM | 981 | NZ | LYS | 136 | 16.053 | 22.914 | 87.323 | 1.00 | 82.43 |
| ATOM | 982 | C | LYS | 136 | 21.548 | 26.044 | 85.429 | 1.00 | 84.12 |
| ATOM | 983 | O | LYS | 136 | 22.185 | 25.610 | 86.390 | 1.00 | 84.51 |
| ATOM | 984 | N | HIS | 137 | 22.119 | 26.727 | 84.442 | 1.00 | 85.08 |
| ATOM | 985 | CA | HIS | 137 | 23.551 | 27.010 | 84.450 | 1.00 | 86.27 |
| ATOM | 986 | CB | HIS | 137 | 24.280 | 26.115 | 83.438 | 1.00 | 86.74 |
| ATOM | 987 | CG | HIS | 137 | 24.169 | 24.649 | 83.730 | 1.00 | 87.04 |
| ATOM | 988 | CD2 | HIS | 137 | 25.112 | 23.729 | 84.047 | 1.00 | 86.44 |
| ATOM | 989 | ND1 | HIS | 137 | 22.968 | 23.971 | 83.708 | 1.00 | 87.51 |
| ATOM | 990 | CE1 | HIS | 137 | 23.176 | 22.699 | 83.999 | 1.00 | 86.59 |
| ATOM | 991 | NE2 | HIS | 137 | 24.468 | 22.526 | 84.209 | 1.00 | 86.35 |
| ATOM | 992 | C | HIS | 137 | 23.820 | 28.476 | 84.123 | 1.00 | 87.11 |
| ATOM | 993 | O | HIS | 137 | 24.943 | 28.842 | 83.776 | 1.00 | 86.73 |
| ATOM | 994 | N | GLN | 138 | 22.784 | 29.307 | 84.249 | 1.00 | 88.41 |
| ATOM | 995 | CA | GLN | 138 | 22.883 | 30.736 | 83.955 | 1.00 | 89.43 |
| ATOM | 996 | CB | GLN | 138 | 23.469 | 31.512 | 85.140 | 1.00 | 90.47 |
| ATOM | 997 | CG | GLN | 138 | 22.654 | 31.451 | 86.419 | 1.00 | 92.10 |
| ATOM | 998 | CD | GLN | 138 | 22.738 | 30.099 | 87.095 | 1.00 | 93.09 |
| ATOM | 999 | OE1 | GLN | 138 | 23.829 | 29.598 | 87.372 | 1.00 | 93.35 |
| ATOM | 1000 | NE2 | GLN | 138 | 21.584 | 29.501 | 87.371 | 1.00 | 93.71 |
| ATOM | 1001 | C | GLN | 138 | 23.779 | 30.931 | 82.747 | 1.00 | 89.90 |
| ATOM | 1002 | O | GLN | 138 | 24.922 | 31.376 | 82.875 | 1.00 | 89.53 |
| ATOM | 1003 | N | MET | 139 | 23.262 | 30.591 | 81.573 | 1.00 | 89.97 |
| ATOM | 1004 | CA | MET | 139 | 24.046 | 30.725 | 80.359 | 1.00 | 90.27 |
| ATOM | 1005 | CB | MET | 139 | 24.995 | 29.529 | 80.235 | 1.00 | 90.82 |
| ATOM | 1006 | CG | MET | 139 | 26.314 | 29.838 | 79.542 | 1.00 | 91.26 |
| ATOM | 1007 | SD | MET | 139 | 27.526 | 28.508 | 79.736 | 1.00 | 90.73 |
| ATOM | 1008 | CE | MET | 139 | 28.303 | 28.974 | 81.303 | 1.00 | 91.08 |
| ATOM | 1009 | C | MET | 139 | 23.137 | 30.820 | 79.140 | 1.00 | 90.17 |
| ATOM | 1010 | O | MET | 139 | 23.610 | 30.894 | 78.006 | 1.00 | 90.11 |
| ATOM | 1011 | N | LYS | 140 | 21.829 | 30.829 | 79.380 | 1.00 | 89.92 |
| ATOM | 1012 | CA | LYS | 140 | 20.851 | 30.921 | 78.300 | 1.00 | 89.78 |
| ATOM | 1013 | CB | LYS | 140 | 19.434 | 30.922 | 78.874 | 1.00 | 89.37 |
| ATOM | 1014 | CG | LYS | 140 | 18.357 | 31.239 | 77.852 | 1.00 | 89.17 |
| ATOM | 1015 | CD | LYS | 140 | 16.972 | 31.055 | 78.438 | 1.00 | 89.06 |
| ATOM | 1016 | CE | LYS | 140 | 16.688 | 29.588 | 78.675 | 1.00 | 88.66 |
| ATOM | 1017 | NZ | LYS | 140 | 16.797 | 28.822 | 77.406 | 1.00 | 88.73 |
| ATOM | 1018 | C | LYS | 140 | 21.067 | 32.179 | 77.466 | 1.00 | 89.78 |
| ATOM | 1019 | O | LYS | 140 | 20.593 | 32.278 | 76.334 | 1.00 | 89.28 |
| ATOM | 1020 | N | HIS | 141 | 21.794 | 33.133 | 78.037 | 1.00 | 90.38 |
| ATOM | 1021 | CA | HIS | 141 | 22.082 | 34.401 | 77.376 | 1.00 | 90.81 |
| ATOM | 1022 | CB | HIS | 141 | 22.222 | 35.506 | 78.427 | 1.00 | 90.98 |
| ATOM | 1023 | CG | HIS | 141 | 23.294 | 35.243 | 79.443 | 1.00 | 91.18 |
| ATOM | 1024 | CD2 | HIS | 141 | 24.520 | 35.794 | 79.610 | 1.00 | 91.04 |
| ATOM | 1025 | ND1 | HIS | 141 | 23.163 | 34.294 | 80.434 | 1.00 | 91.11 |
| ATOM | 1026 | CE1 | HIS | 141 | 24.262 | 34.273 | 81.168 | 1.00 | 91.45 |
| ATOM | 1027 | NE2 | HIS | 141 | 25.102 | 35.174 | 80.688 | 1.00 | 90.96 |
| ATOM | 1028 | C | HIS | 141 | 23.349 | 34.367 | 76.516 | 1.00 | 90.72 |
| ATOM | 1029 | O | HIS | 141 | 24.048 | 35.374 | 76.399 | 1.00 | 91.00 |
| ATOM | 1030 | N | LYS | 142 | 23.648 | 33.220 | 75.912 | 1.00 | 90.17 |
| ATOM | 1031 | CA | LYS | 142 | 24.845 | 33.109 | 75.082 | 1.00 | 89.12 |
| ATOM | 1032 | CB | LYS | 142 | 26.000 | 32.529 | 75.908 | 1.00 | 89.54 |
| ATOM | 1033 | CG | LYS | 142 | 26.424 | 33.406 | 77.079 | 1.00 | 90.51 |
| ATOM | 1034 | CD | LYS | 142 | 27.490 | 32.730 | 77.926 | 1.00 | 91.91 |
| ATOM | 1035 | CE | LYS | 142 | 27.867 | 33.579 | 79.131 | 1.00 | 92.42 |
| ATOM | 1036 | NZ | LYS | 142 | 28.820 | 32.863 | 80.026 | 1.00 | 92.34 |
| ATOM | 1037 | C | LYS | 142 | 24.643 | 32.276 | 73.815 | 1.00 | 87.58 |
| ATOM | 1038 | O | LYS | 142 | 23.763 | 31.418 | 73.749 | 1.00 | 87.74 |
| ATOM | 1039 | N | LYS | 143 | 25.465 | 32.554 | 72.808 | 1.00 | 85.65 |
| ATOM | 1040 | CA | LYS | 143 | 25.414 | 31.849 | 71.532 | 1.00 | 83.45 |
| ATOM | 1041 | CB | LYS | 143 | 25.052 | 32.819 | 70.402 | 1.00 | 83.10 |
| ATOM | 1042 | CG | LYS | 143 | 25.199 | 32.262 | 68.988 | 1.00 | 82.55 |
| ATOM | 1043 | CD | LYS | 143 | 24.890 | 33.339 | 67.951 | 1.00 | 82.36 |
| ATOM | 1044 | CE | LYS | 143 | 25.289 | 32.922 | 66.540 | 1.00 | 82.46 |
| ATOM | 1045 | NZ | LYS | 143 | 24.519 | 31.749 | 66.045 | 1.00 | 82.29 |
| ATOM | 1046 | C | LYS | 143 | 26.790 | 31.252 | 71.283 | 1.00 | 82.32 |
| ATOM | 1047 | O | LYS | 143 | 27.751 | 31.974 | 71.002 | 1.00 | 82.33 |
| ATOM | 1048 | N | LEU | 144 | 26.884 | 29.932 | 71.409 | 1.00 | 79.90 |
| ATOM | 1049 | CA | LEU | 144 | 28.146 | 29.233 | 71.198 | 1.00 | 77.12 |
| ATOM | 1050 | CB | LEU | 144 | 28.653 | 28.634 | 72.517 | 1.00 | 78.89 |
| ATOM | 1051 | CG | LEU | 144 | 29.417 | 29.543 | 73.491 | 1.00 | 80.11 |
| ATOM | 1052 | CD1 | LEU | 144 | 28.560 | 30.727 | 73.924 | 1.00 | 81.77 |
| ATOM | 1053 | CD2 | LEU | 144 | 29.836 | 28.721 | 74.698 | 1.00 | 80.96 |
| ATOM | 1054 | C | LEU | 144 | 27.993 | 28.132 | 70.156 | 1.00 | 73.23 |
| ATOM | 1055 | O | LEU | 144 | 26.876 | 27.742 | 69.810 | 1.00 | 72.89 |
| ATOM | 1056 | N | PRO | 145 | 29.119 | 27.628 | 69.628 | 1.00 | 70.01 |
| ATOM | 1057 | CD | PRO | 145 | 30.498 | 28.104 | 69.833 | 1.00 | 68.83 |
| ATOM | 1058 | CA | PRO | 145 | 29.081 | 26.565 | 68.621 | 1.00 | 67.77 |
| ATOM | 1059 | CB | PRO | 145 | 30.555 | 26.356 | 68.285 | 1.00 | 68.79 |
| ATOM | 1060 | CG | PRO | 145 | 31.159 | 27.706 | 68.542 | 1.00 | 69.21 |
| ATOM | 1061 | C | PRO | 145 | 28.434 | 25.299 | 69.181 | 1.00 | 65.49 |
| ATOM | 1062 | O | PRO | 145 | 28.615 | 24.963 | 70.351 | 1.00 | 64.23 |
| ATOM | 1063 | N | LEU | 146 | 27.677 | 24.603 | 68.340 | 1.00 | 63.31 |
| ATOM | 1064 | CA | LEU | 146 | 27.007 | 23.383 | 68.757 | 1.00 | 61.72 |
| ATOM | 1065 | CB | LEU | 146 | 25.492 | 23.532 | 68.602 | 1.00 | 62.15 |
| ATOM | 1066 | CG | LEU | 146 | 24.678 | 22.285 | 68.945 | 1.00 | 62.90 |
| ATOM | 1067 | CD1 | LEU | 146 | 25.011 | 21.842 | 70.353 | 1.00 | 64.57 |
| ATOM | 1068 | CD2 | LEU | 146 | 23.194 | 22.577 | 68.817 | 1.00 | 65.06 |
| ATOM | 1069 | C | LEU | 146 | 27.473 | 22.152 | 67.985 | 1.00 | 59.94 |
| ATOM | 1070 | O | LEU | 146 | 27.342 | 22.086 | 66.763 | 1.00 | 59.04 |
| ATOM | 1071 | N | GLY | 147 | 28.028 | 21.189 | 68.721 | 1.00 | 58.65 |
| ATOM | 1072 | CA | GLY | 147 | 28.492 | 19.939 | 68.136 | 1.00 | 54.15 |
| ATOM | 1073 | C | GLY | 147 | 27.444 | 18.891 | 68.465 | 1.00 | 49.71 |
| ATOM | 1074 | O | GLY | 147 | 27.175 | 18.628 | 69.635 | 1.00 | 50.70 |
| ATOM | 1075 | N | PHE | 148 | 26.854 | 18.287 | 67.440 | 1.00 | 46.12 |
| ATOM | 1076 | CA | PHE | 148 | 25.795 | 17.297 | 67.635 | 1.00 | 42.39 |
| ATOM | 1077 | CB | PHE | 148 | 24.610 | 17.675 | 66.740 | 1.00 | 39.68 |
| ATOM | 1078 | CG | PHE | 148 | 23.366 | 16.864 | 66.977 | 1.00 | 38.24 |
| ATOM | 1079 | CD1 | PHE | 148 | 22.326 | 16.901 | 66.056 | 1.00 | 36.04 |
| ATOM | 1080 | CD2 | PHE | 148 | 23.212 | 16.102 | 68.132 | 1.00 | 36.13 |
| ATOM | 1081 | CE1 | PHE | 148 | 21.148 | 16.194 | 66.279 | 1.00 | 38.53 |
| ATOM | 1082 | CE2 | PHE | 148 | 22.042 | 15.395 | 68.365 | 1.00 | 35.28 |
| ATOM | 1083 | CZ | PHE | 148 | 21.005 | 15.440 | 67.437 | 1.00 | 37.48 |
| ATOM | 1084 | C | PHE | 148 | 26.197 | 15.840 | 67.354 | 1.00 | 41.67 |
| ATOM | 1085 | O | PHE | 148 | 26.463 | 15.475 | 66.205 | 1.00 | 42.24 |
| ATOM | 1086 | N | THR | 149 | 26.247 | 15.013 | 68.398 | 1.00 | 40.23 |
| ATOM | 1087 | CA | THR | 149 | 26.562 | 13.593 | 68.222 | 1.00 | 36.30 |
| ATOM | 1088 | CB | THR | 149 | 27.281 | 13.001 | 69.442 | 1.00 | 36.36 |
| ATOM | 1089 | OG1 | THR | 149 | 28.580 | 13.597 | 69.560 | 1.00 | 37.54 |
| ATOM | 1090 | CG2 | THR | 149 | 27.444 | 11.492 | 69.286 | 1.00 | 37.01 |
| ATOM | 1091 | C | THR | 149 | 25.212 | 12.909 | 68.039 | 1.00 | 34.65 |
| ATOM | 1092 | O | THR | 149 | 24.412 | 12.836 | 68.967 | 1.00 | 31.13 |
| ATOM | 1093 | N | PHE | 150 | 24.972 | 12.422 | 66.825 | 1.00 | 33.67 |
| ATOM | 1094 | CA | PHE | 150 | 23.714 | 11.782 | 66.456 | 1.00 | 34.60 |
| ATOM | 1095 | CB | PHE | 150 | 23.061 | 12.614 | 65.336 | 1.00 | 32.78 |
| ATOM | 1096 | CG | PHE | 150 | 21.739 | 12.086 | 64.854 | 1.00 | 30.57 |
| ATOM | 1097 | CD1 | PHE | 150 | 21.625 | 11.513 | 63.595 | 1.00 | 30.43 |
| ATOM | 1098 | CD2 | PHE | 150 | 20.598 | 12.213 | 65.637 | 1.00 | 31.90 |
| ATOM | 1099 | CE1 | PHE | 150 | 20.382 | 11.076 | 63.115 | 1.00 | 34.54 |
| ATOM | 1100 | CE2 | PHE | 150 | 19.356 | 11.783 | 65.176 | 1.00 | 30.63 |
| ATOM | 1101 | CZ | PHE | 150 | 19.241 | 11.213 | 63.913 | 1.00 | 32.01 |
| ATOM | 1102 | C | PHE | 150 | 24.011 | 10.358 | 65.991 | 1.00 | 35.95 |
| ATOM | 1103 | O | PHE | 150 | 24.369 | 10.128 | 64.836 | 1.00 | 38.42 |
| ATOM | 1104 | N | SER | 151 | 23.843 | 9.412 | 66.908 | 1.00 | 36.96 |
| ATOM | 1105 | CA | SER | 151 | 24.129 | 7.995 | 66.680 | 1.00 | 34.37 |
| ATOM | 1106 | CB | SER | 151 | 24.186 | 7.271 | 68.025 | 1.00 | 35.80 |
| ATOM | 1107 | OG | SER | 151 | 25.111 | 7.897 | 68.892 | 1.00 | 39.97 |
| ATOM | 1108 | C | SER | 151 | 23.189 | 7.228 | 65.770 | 1.00 | 32.05 |
| ATOM | 1109 | O | SER | 151 | 22.537 | 6.292 | 66.215 | 1.00 | 32.11 |
| ATOM | 1110 | N | PHE | 152 | 23.110 | 7.611 | 64.505 | 1.00 | 31.41 |
| ATOM | 1111 | CA | PHE | 152 | 22.253 | 6.902 | 63.563 | 1.00 | 31.81 |
| ATOM | 1112 | CB | PHE | 152 | 20.824 | 7.464 | 63.570 | 1.00 | 34.43 |
| ATOM | 1113 | CG | PHE | 152 | 20.149 | 7.372 | 64.904 | 1.00 | 34.95 |
| ATOM | 1114 | CD1 | PHE | 152 | 20.278 | 8.401 | 65.838 | 1.00 | 32.95 |
| ATOM | 1115 | CD2 | PHE | 152 | 19.439 | 6.228 | 65.256 | 1.00 | 35.34 |
| ATOM | 1116 | CE1 | PHE | 152 | 19.713 | 8.291 | 67.108 | 1.00 | 35.00 |
| ATOM | 1117 | CE2 | PHE | 152 | 18.868 | 6.102 | 66.526 | 1.00 | 35.79 |
| ATOM | 1118 | CZ | PHE | 152 | 19.005 | 7.135 | 67.454 | 1.00 | 38.15 |
| ATOM | 1119 | C | PHE | 152 | 22.845 | 7.010 | 62.171 | 1.00 | 31.95 |
| ATOM | 1120 | O | PHE | 152 | 23.727 | 7.831 | 61.921 | 1.00 | 31.72 |
| ATOM | 1121 | N | PRO | 153 | 22.386 | 6.164 | 61.247 | 1.00 | 32.44 |
| ATOM | 1122 | CD | PRO | 153 | 21.374 | 5.098 | 61.343 | 1.00 | 30.73 |
| ATOM | 1123 | CA | PRO | 153 | 22.942 | 6.248 | 59.896 | 1.00 | 34.59 |
| ATOM | 1124 | CB | PRO | 153 | 22.397 | 4.991 | 59.225 | 1.00 | 31.34 |
| ATOM | 1125 | CG | PRO | 153 | 21.072 | 4.812 | 59.884 | 1.00 | 31.98 |
| ATOM | 1126 | C | PRO | 153 | 22.507 | 7.535 | 59.201 | 1.00 | 37.30 |
| ATOM | 1127 | O | PRO | 153 | 21.310 | 7.813 | 59.067 | 1.00 | 39.02 |
| ATOM | 1128 | N | VAL | 154 | 23.483 | 8.325 | 58.770 | 1.00 | 39.02 |
| ATOM | 1129 | CA | VAL | 154 | 23.187 | 9.581 | 58.092 | 1.00 | 40.43 |
| ATOM | 1130 | CB | VAL | 154 | 23.446 | 10.792 | 59.007 | 1.00 | 39.28 |
| ATOM | 1131 | CG1 | VAL | 154 | 23.191 | 12.081 | 58.238 | 1.00 | 41.18 |
| ATOM | 1132 | CG2 | VAL | 154 | 22.557 | 10.727 | 60.221 | 1.00 | 38.37 |
| ATOM | 1133 | C | VAL | 154 | 24.023 | 9.785 | 56.837 | 1.00 | 41.48 |
| ATOM | 1134 | O | VAL | 154 | 25.241 | 9.602 | 56.861 | 1.00 | 41.28 |
| ATOM | 1135 | N | ARG | 155 | 23.365 | 10.162 | 55.743 | 1.00 | 43.31 |
| ATOM | 1136 | CA | ARG | 155 | 24.072 | 10.441 | 54.495 | 1.00 | 46.32 |
| ATOM | 1137 | CB | ARG | 155 | 23.233 | 10.058 | 53.280 | 1.00 | 47.31 |
| ATOM | 1138 | CG | ARG | 155 | 23.809 | 10.586 | 51.968 | 1.00 | 52.20 |
| ATOM | 1139 | CD | ARG | 155 | 23.563 | 9.614 | 50.844 | 1.00 | 55.56 |
| ATOM | 1140 | NE | ARG | 155 | 24.419 | 8.437 | 50.968 | 1.00 | 59.93 |
| ATOM | 1141 | CZ | ARG | 155 | 24.068 | 7.217 | 50.573 | 1.00 | 61.41 |
| ATOM | 1142 | NH1 | ARG | 155 | 22.874 | 7.011 | 50.032 | 1.00 | 63.00 |
| ATOM | 1143 | NH2 | ARG | 155 | 24.910 | 6.203 | 50.717 | 1.00 | 63.35 |
| ATOM | 1144 | C | ARG | 155 | 24.367 | 11.934 | 54.456 | 1.00 | 46.23 |
| ATOM | 1145 | O | ARG | 155 | 23.486 | 12.737 | 54.166 | 1.00 | 47.64 |
| ATOM | 1146 | N | HIS | 156 | 25.613 | 12.291 | 54.754 | 1.00 | 47.03 |
| ATOM | 1147 | CA | HIS | 156 | 26.046 | 13.682 | 54.791 | 1.00 | 48.05 |
| ATOM | 1148 | CB | HIS | 156 | 27.318 | 13.834 | 55.632 | 1.00 | 49.62 |
| ATOM | 1149 | CG | HIS | 156 | 27.157 | 13.444 | 57.066 | 1.00 | 52.65 |
| ATOM | 1150 | CD2 | HIS | 156 | 26.274 | 12.619 | 57.676 | 1.00 | 53.99 |
| ATOM | 1151 | ND1 | HIS | 156 | 27.990 | 13.916 | 58.057 | 1.00 | 53.35 |
| ATOM | 1152 | CE1 | HIS | 156 | 27.625 | 13.401 | 59.218 | 1.00 | 54.78 |
| ATOM | 1153 | NE2 | HIS | 156 | 26.586 | 12.610 | 59.014 | 1.00 | 54.28 |
| ATOM | 1154 | C | HIS | 156 | 26.334 | 14.317 | 53.440 | 1.00 | 48.30 |
| ATOM | 1155 | O | HIS | 156 | 26.872 | 13.677 | 52.535 | 1.00 | 47.38 |
| ATOM | 1156 | N | GLU | 157 | 25.969 | 15.589 | 53.319 | 1.00 | 47.98 |
| ATOM | 1157 | CA | GLU | 157 | 26.256 | 16.343 | 52.114 | 1.00 | 48.38 |
| ATOM | 1158 | CB | GLU | 157 | 25.113 | 17.296 | 51.749 | 1.00 | 51.05 |
| ATOM | 1159 | CG | GLU | 157 | 25.462 | 18.198 | 50.558 | 1.00 | 57.22 |
| ATOM | 1160 | CD | GLU | 157 | 24.422 | 19.276 | 50.275 | 1.00 | 58.92 |
| ATOM | 1161 | OE1 | GLU | 157 | 23.299 | 18.931 | 49.845 | 1.00 | 60.91 |
| ATOM | 1162 | OE2 | GLU | 157 | 24.734 | 20.471 | 50.485 | 1.00 | 60.69 |
| ATOM | 1163 | C | GLU | 157 | 27.475 | 17.138 | 52.547 | 1.00 | 45.12 |
| ATOM | 1164 | O | GLU | 157 | 28.349 | 17.457 | 51.749 | 1.00 | 43.91 |
| ATOM | 1165 | N | ASP | 158 | 27.529 | 17.427 | 53.843 | 1.00 | 43.85 |
| ATOM | 1166 | CA | ASP | 158 | 28.633 | 18.174 | 54.416 | 1.00 | 43.81 |
| ATOM | 1167 | CB | ASP | 158 | 28.479 | 19.654 | 54.085 | 1.00 | 46.74 |
| ATOM | 1168 | CG | ASP | 158 | 29.743 | 20.445 | 54.349 | 1.00 | 49.54 |
| ATOM | 1169 | OD1 | ASP | 158 | 29.760 | 21.651 | 54.016 | 1.00 | 53.34 |
| ATOM | 1170 | OD2 | ASP | 158 | 30.716 | 19.869 | 54.884 | 1.00 | 49.62 |
| ATOM | 1171 | C | ASP | 158 | 28.671 | 17.972 | 55.928 | 1.00 | 43.93 |
| ATOM | 1172 | O | ASP | 158 | 27.724 | 17.447 | 56.518 | 1.00 | 43.97 |
| ATOM | 1173 | N | ILE | 159 | 29.767 | 18.399 | 56.547 | 1.00 | 43.75 |
| ATOM | 1174 | CA | ILE | 159 | 29.963 | 18.250 | 57.983 | 1.00 | 44.27 |
| ATOM | 1175 | CB | ILE | 159 | 31.248 | 18.971 | 58.452 | 1.00 | 45.07 |
| ATOM | 1176 | CG2 | ILE | 159 | 31.069 | 20.480 | 58.354 | 1.00 | 47.24 |
| ATOM | 1177 | CG1 | ILE | 159 | 31.544 | 18.617 | 59.907 | 1.00 | 45.29 |
| ATOM | 1178 | CD1 | ILE | 159 | 31.733 | 17.140 | 60.152 | 1.00 | 48.99 |
| ATOM | 1179 | C | ILE | 159 | 28.795 | 18.744 | 58.829 | 1.00 | 44.47 |
| ATOM | 1180 | O | ILE | 159 | 28.583 | 18.254 | 59.941 | 1.00 | 44.15 |
| ATOM | 1181 | N | ASP | 160 | 28.037 | 19.709 | 58.317 | 1.00 | 44.10 |
| ATOM | 1182 | CA | ASP | 160 | 26.904 | 20.239 | 59.072 | 1.00 | 42.92 |
| ATOM | 1183 | CB | ASP | 160 | 27.103 | 21.734 | 59.360 | 1.00 | 44.13 |
| ATOM | 1184 | CG | ASP | 160 | 27.448 | 22.533 | 58.118 | 1.00 | 45.52 |
| ATOM | 1185 | OD1 | ASP | 160 | 28.258 | 23.479 | 58.239 | 1.00 | 47.57 |
| ATOM | 1186 | OD2 | ASP | 160 | 26.912 | 22.228 | 57.031 | 1.00 | 45.51 |
| ATOM | 1187 | C | ASP | 160 | 25.559 | 20.005 | 58.410 | 1.00 | 42.92 |
| ATOM | 1188 | O | ASP | 160 | 24.579 | 20.691 | 58.706 | 1.00 | 44.48 |
| ATOM | 1189 | N | LYS | 161 | 25.509 | 19.026 | 57.518 | 1.00 | 41.57 |
| ATOM | 1190 | CA | LYS | 161 | 24.267 | 18.692 | 56.838 | 1.00 | 41.51 |
| ATOM | 1191 | CB | LYS | 161 | 24.067 | 19.597 | 55.618 | 1.00 | 41.19 |
| ATOM | 1192 | CG | LYS | 161 | 22.783 | 19.306 | 54.863 | 1.00 | 41.39 |
| ATOM | 1193 | CD | LYS | 161 | 22.687 | 20.094 | 53.557 | 1.00 | 43.25 |
| ATOM | 1194 | CE | LYS | 161 | 21.366 | 19.809 | 52.860 | 1.00 | 40.06 |
| ATOM | 1195 | NZ | LYS | 161 | 21.335 | 20.312 | 51.468 | 1.00 | 41.02 |
| ATOM | 1196 | C | LYS | 161 | 24.258 | 17.224 | 56.397 | 1.00 | 41.66 |
| ATOM | 1197 | O | LYS | 161 | 25.239 | 16.725 | 55.838 | 1.00 | 39.36 |
| ATOM | 1198 | N | GLY | 162 | 23.143 | 16.546 | 56.654 | 1.00 | 40.90 |
| ATOM | 1199 | CA | GLY | 162 | 23.005 | 15.152 | 56.276 | 1.00 | 42.70 |
| ATOM | 1200 | C | GLY | 162 | 21.618 | 14.645 | 56.615 | 1.00 | 43.15 |
| ATOM | 1201 | O | GLY | 162 | 21.019 | 15.085 | 57.594 | 1.00 | 43.59 |
| ATOM | 1202 | N | ILE | 163 | 21.096 | 13.722 | 55.816 | 1.00 | 43.93 |
| ATOM | 1203 | CA | ILE | 163 | 19.763 | 13.190 | 56.068 | 1.00 | 45.03 |
| ATOM | 1204 | CB | ILE | 163 | 18.958 | 13.031 | 54.755 | 1.00 | 46.16 |
| ATOM | 1205 | CG2 | ILE | 163 | 18.943 | 14.352 | 53.985 | 1.00 | 45.82 |
| ATOM | 1206 | CG1 | ILE | 163 | 19.585 | 11.938 | 53.889 | 1.00 | 46.11 |
| ATOM | 1207 | CD1 | ILE | 163 | 18.812 | 11.638 | 52.613 | 1.00 | 48.51 |
| ATOM | 1208 | C | ILE | 163 | 19.812 | 11.833 | 56.764 | 1.00 | 46.49 |
| ATOM | 1209 | O | ILE | 163 | 20.771 | 11.074 | 56.609 | 1.00 | 45.36 |
| ATOM | 1210 | N | LEU | 164 | 18.767 | 11.545 | 57.533 | 1.00 | 47.21 |
| ATOM | 1211 | CA | LEU | 164 | 18.649 | 10.286 | 58.253 | 1.00 | 47.53 |
| ATOM | 1212 | CB | LEU | 164 | 17.623 | 10.414 | 59.379 | 1.00 | 47.11 |
| ATOM | 1213 | CG | LEU | 164 | 17.135 | 9.126 | 60.049 | 1.00 | 47.15 |
| ATOM | 1214 | CD1 | LEU | 164 | 18.265 | 8.469 | 60.832 | 1.00 | 45.45 |
| ATOM | 1215 | CD2 | LEU | 164 | 15.981 | 9.465 | 60.977 | 1.00 | 47.00 |
| ATOM | 1216 | C | LEU | 164 | 18.189 | 9.220 | 57.277 | 1.00 | 48.06 |
| ATOM | 1217 | O | LEU | 164 | 17.137 | 9.352 | 56.657 | 1.00 | 48.49 |
| ATOM | 1218 | N | LEU | 165 | 18.977 | 8.161 | 57.137 | 1.00 | 48.32 |
| ATOM | 1219 | CA | LEU | 165 | 18.614 | 7.093 | 56.224 | 1.00 | 47.47 |
| ATOM | 1220 | CB | LEU | 165 | 19.827 | 6.208 | 55.954 | 1.00 | 45.44 |
| ATOM | 1221 | CG | LEU | 165 | 20.867 | 6.978 | 55.140 | 1.00 | 47.60 |
| ATOM | 1222 | CD1 | LEU | 165 | 22.128 | 6.155 | 54.956 | 1.00 | 47.47 |
| ATOM | 1223 | CD2 | LEU | 165 | 20.261 | 7.342 | 53.786 | 1.00 | 48.48 |
| ATOM | 1224 | C | LEU | 165 | 17.460 | 6.300 | 56.814 | 1.00 | 46.86 |
| ATOM | 1225 | O | LEU | 165 | 16.497 | 5.985 | 56.120 | 1.00 | 46.90 |
| ATOM | 1226 | N | ASN | 166 | 17.562 | 5.992 | 58.101 | 1.00 | 46.60 |
| ATOM | 1227 | CA | ASN | 166 | 16.521 | 5.266 | 58.817 | 1.00 | 47.23 |
| ATOM | 1228 | CB | ASN | 166 | 16.282 | 3.883 | 58.200 | 1.00 | 49.17 |
| ATOM | 1229 | CG | ASN | 166 | 17.542 | 3.053 | 58.118 | 1.00 | 50.36 |
| ATOM | 1230 | OD1 | ASN | 166 | 18.205 | 2.997 | 57.076 | 1.00 | 50.62 |
| ATOM | 1231 | ND2 | ASN | 166 | 17.888 | 2.406 | 59.223 | 1.00 | 50.50 |
| ATOM | 1232 | C | ASN | 166 | 16.913 | 5.123 | 60.279 | 1.00 | 47.60 |
| ATOM | 1233 | O | ASN | 166 | 18.096 | 5.177 | 60.623 | 1.00 | 48.53 |
| ATOM | 1234 | N | TRP | 167 | 15.916 | 4.966 | 61.142 | 1.00 | 46.96 |
| ATOM | 1235 | CA | TRP | 167 | 16.166 | 4.815 | 62.571 | 1.00 | 45.46 |
| ATOM | 1236 | CB | TRP | 167 | 14.890 | 5.085 | 63.376 | 1.00 | 47.63 |
| ATOM | 1237 | CG | TRP | 167 | 14.433 | 6.519 | 63.454 | 1.00 | 49.15 |
| ATOM | 1238 | CD2 | TRP | 167 | 15.093 | 7.602 | 64.126 | 1.00 | 49.07 |
| ATOM | 1239 | CE2 | TRP | 167 | 14.237 | 8.725 | 64.050 | 1.00 | 48.21 |
| ATOM | 1240 | CE3 | TRP | 167 | 16.321 | 7.732 | 64.787 | 1.00 | 49.17 |
| ATOM | 1241 | CD1 | TRP | 167 | 13.242 | 7.022 | 63.003 | 1.00 | 49.03 |
| ATOM | 1242 | NE1 | TRP | 167 | 13.117 | 8.343 | 63.361 | 1.00 | 48.46 |
| ATOM | 1243 | CZ2 | TRP | 167 | 14.569 | 9.962 | 64.614 | 1.00 | 47.68 |
| ATOM | 1244 | CZ3 | TRP | 167 | 16.652 | 8.966 | 65.348 | 1.00 | 49.58 |
| ATOM | 1245 | CH2 | TRP | 167 | 15.777 | 10.064 | 65.256 | 1.00 | 48.80 |
| ATOM | 1246 | C | TRP | 167 | 16.647 | 3.394 | 62.890 | 1.00 | 43.28 |
| ATOM | 1247 | O | TRP | 167 | 16.425 | 2.461 | 62.119 | 1.00 | 42.86 |
| ATOM | 1248 | N | THR | 168 | 17.297 | 3.245 | 64.038 | 1.00 | 41.63 |
| ATOM | 1249 | CA | THR | 168 | 17.796 | 1.953 | 64.501 | 1.00 | 40.13 |
| ATOM | 1250 | CB | THR | 168 | 19.275 | 1.723 | 64.086 | 1.00 | 37.87 |
| ATOM | 1251 | OG1 | THR | 168 | 20.082 | 2.795 | 64.587 | 1.00 | 33.52 |
| ATOM | 1252 | CG2 | THR | 168 | 19.417 | 1.647 | 62.566 | 1.00 | 34.11 |
| ATOM | 1253 | C | THR | 168 | 17.719 | 1.943 | 66.029 | 1.00 | 41.33 |
| ATOM | 1254 | O | THR | 168 | 17.382 | 2.953 | 66.649 | 1.00 | 41.41 |
| ATOM | 1255 | N | LYS | 169 | 18.025 | 0.799 | 66.631 | 1.00 | 42.06 |
| ATOM | 1256 | CA | LYS | 169 | 18.013 | 0.672 | 68.083 | 1.00 | 42.59 |
| ATOM | 1257 | CB | LYS | 169 | 19.077 | 1.594 | 68.683 | 1.00 | 39.56 |
| ATOM | 1258 | CG | LYS | 169 | 20.497 | 1.209 | 68.287 | 1.00 | 36.24 |
| ATOM | 1259 | CD | LYS | 169 | 21.528 | 2.170 | 68.840 | 1.00 | 33.26 |
| ATOM | 1260 | CE | LYS | 169 | 21.481 | 3.514 | 68.133 | 1.00 | 30.26 |
| ATOM | 1261 | NZ | LYS | 169 | 22.589 | 4.373 | 68.610 | 1.00 | 32.75 |
| ATOM | 1262 | C | LYS | 169 | 16.661 | 0.933 | 68.751 | 1.00 | 45.26 |
| ATOM | 1263 | O | LYS | 169 | 16.598 | 1.191 | 69.955 | 1.00 | 45.85 |
| ATOM | 1264 | N | GLY | 170 | 15.583 | 0.881 | 67.975 | 1.00 | 47.46 |
| ATOM | 1265 | CA | GLY | 170 | 14.267 | 1.083 | 68.555 | 1.00 | 52.44 |
| ATOM | 1266 | C | GLY | 170 | 13.552 | 2.394 | 68.295 | 1.00 | 55.24 |
| ATOM | 1267 | O | GLY | 170 | 12.324 | 2.422 | 68.275 | 1.00 | 56.11 |
| ATOM | 1268 | N | PHE | 171 | 14.293 | 3.482 | 68.118 | 1.00 | 58.10 |
| ATOM | 1269 | CA | PHE | 171 | 13.668 | 4.777 | 67.861 | 1.00 | 61.86 |
| ATOM | 1270 | CB | PHE | 171 | 14.734 | 5.846 | 67.613 | 1.00 | 62.35 |
| ATOM | 1271 | CG | PHE | 171 | 15.449 | 6.285 | 68.856 | 1.00 | 64.08 |
| ATOM | 1272 | CD1 | PHE | 171 | 16.060 | 5.354 | 69.691 | 1.00 | 66.00 |
| ATOM | 1273 | CD2 | PHE | 171 | 15.511 | 7.630 | 69.196 | 1.00 | 64.53 |
| ATOM | 1274 | CE1 | PHE | 171 | 16.721 | 5.760 | 70.851 | 1.00 | 66.96 |
| ATOM | 1275 | CE2 | PHE | 171 | 16.170 | 8.046 | 70.352 | 1.00 | 65.93 |
| ATOM | 1276 | CZ | PHE | 171 | 16.776 | 7.109 | 71.180 | 1.00 | 66.07 |
| ATOM | 1277 | C | PHE | 171 | 12.727 | 4.697 | 66.663 | 1.00 | 63.91 |
| ATOM | 1278 | O | PHE | 171 | 12.994 | 3.975 | 65.702 | 1.00 | 63.50 |
| ATOM | 1279 | N | LYS | 172 | 11.620 | 5.430 | 66.727 | 1.00 | 65.77 |
| ATOM | 1280 | CA | LYS | 172 | 10.657 | 5.424 | 65.633 | 1.00 | 68.37 |
| ATOM | 1281 | CB | LYS | 172 | 9.738 | 4.197 | 65.727 | 1.00 | 70.16 |
| ATOM | 1282 | CG | LYS | 172 | 8.814 | 4.035 | 64.517 | 1.00 | 72.04 |
| ATOM | 1283 | CD | LYS | 172 | 7.867 | 2.842 | 64.647 | 1.00 | 73.43 |
| ATOM | 1284 | CE | LYS | 172 | 6.977 | 2.718 | 63.406 | 1.00 | 74.42 |
| ATOM | 1285 | NZ | LYS | 172 | 5.933 | 1.655 | 63.525 | 1.00 | 73.62 |
| ATOM | 1286 | C | LYS | 172 | 9.808 | 6.688 | 65.606 | 1.00 | 69.18 |
| ATOM | 1287 | O | LYS | 172 | 8.599 | 6.642 | 65.838 | 1.00 | 70.01 |
| ATOM | 1288 | N | ALA | 173 | 10.445 | 7.820 | 65.332 | 1.00 | 68.98 |
| ATOM | 1289 | CA | ALA | 173 | 9.734 | 9.086 | 65.251 | 1.00 | 69.07 |
| ATOM | 1290 | CB | ALA | 173 | 10.598 | 10.210 | 65.818 | 1.00 | 68.41 |
| ATOM | 1291 | C | ALA | 173 | 9.424 | 9.339 | 63.776 | 1.00 | 69.07 |
| ATOM | 1292 | O | ALA | 173 | 10.336 | 9.471 | 62.962 | 1.00 | 69.61 |
| ATOM | 1293 | N | SER | 174 | 8.139 | 9.394 | 63.432 | 1.00 | 69.06 |
| ATOM | 1294 | CA | SER | 174 | 7.735 | 9.620 | 62.047 | 1.00 | 68.32 |
| ATOM | 1295 | CB | SER | 174 | 6.217 | 9.491 | 61.901 | 1.00 | 69.02 |
| ATOM | 1296 | OG | SER | 174 | 5.546 | 10.503 | 62.632 | 1.00 | 68.18 |
| ATOM | 1297 | C | SER | 174 | 8.173 | 10.996 | 61.568 | 1.00 | 67.71 |
| ATOM | 1298 | O | SER | 174 | 8.410 | 11.897 | 62.370 | 1.00 | 68.23 |
| ATOM | 1299 | N | GLY | 175 | 8.288 | 11.148 | 60.254 | 1.00 | 67.37 |
| ATOM | 1300 | CA | GLY | 175 | 8.688 | 12.424 | 59.690 | 1.00 | 67.08 |
| ATOM | 1301 | C | GLY | 175 | 10.143 | 12.787 | 59.915 | 1.00 | 66.86 |
| ATOM | 1302 | O | GLY | 175 | 10.507 | 13.962 | 59.855 | 1.00 | 67.38 |
| ATOM | 1303 | N | ALA | 176 | 10.979 | 11.786 | 60.172 | 1.00 | 66.42 |
| ATOM | 1304 | CA | ALA | 176 | 12.400 | 12.018 | 60.401 | 1.00 | 64.67 |
| ATOM | 1305 | CB | ALA | 176 | 12.828 | 11.360 | 61.699 | 1.00 | 64.11 |
| ATOM | 1306 | C | ALA | 176 | 13.229 | 11.475 | 59.242 | 1.00 | 64.02 |
| ATOM | 1307 | O | ALA | 176 | 14.053 | 12.183 | 58.667 | 1.00 | 65.00 |
| ATOM | 1308 | N | GLU | 177 | 12.993 | 10.214 | 58.903 | 1.00 | 63.24 |
| ATOM | 1309 | CA | GLU | 177 | 13.710 | 9.544 | 57.825 | 1.00 | 63.08 |
| ATOM | 1310 | CB | GLU | 177 | 13.147 | 8.127 | 57.639 | 1.00 | 62.97 |
| ATOM | 1311 | CG | GLU | 177 | 13.315 | 7.224 | 58.865 | 1.00 | 64.81 |
| ATOM | 1312 | CD | GLU | 177 | 12.712 | 5.837 | 58.682 | 1.00 | 64.99 |
| ATOM | 1313 | OE1 | GLU | 177 | 12.948 | 4.972 | 59.552 | 1.00 | 65.80 |
| ATOM | 1314 | OE2 | GLU | 177 | 12.003 | 5.612 | 57.677 | 1.00 | 64.52 |
| ATOM | 1315 | C | GLU | 177 | 13.669 | 10.293 | 56.491 | 1.00 | 62.92 |
| ATOM | 1316 | O | GLU | 177 | 12.602 | 10.489 | 55.908 | 1.00 | 63.26 |
| ATOM | 1317 | N | GLY | 178 | 14.838 | 10.708 | 56.013 | 1.00 | 62.46 |
| ATOM | 1318 | CA | GLY | 178 | 14.911 | 11.406 | 54.741 | 1.00 | 61.36 |
| ATOM | 1319 | C | GLY | 178 | 15.095 | 12.911 | 54.805 | 1.00 | 60.52 |
| ATOM | 1320 | O | GLY | 178 | 15.337 | 13.539 | 53.777 | 1.00 | 61.73 |
| ATOM | 1321 | N | ASN | 179 | 14.990 | 13.498 | 55.993 | 1.00 | 59.84 |
| ATOM | 1322 | CA | ASN | 179 | 15.139 | 14.942 | 56.134 | 1.00 | 59.11 |
| ATOM | 1323 | CB | ASN | 179 | 13.985 | 15.512 | 56.959 | 1.00 | 59.72 |
| ATOM | 1324 | CG | ASN | 179 | 12.630 | 15.217 | 56.342 | 1.00 | 61.46 |
| ATOM | 1325 | OD1 | ASN | 179 | 12.423 | 15.416 | 55.143 | 1.00 | 61.86 |
| ATOM | 1326 | ND2 | ASN | 179 | 11.696 | 14.743 | 57.161 | 1.00 | 61.02 |
| ATOM | 1327 | C | ASN | 179 | 16.463 | 15.349 | 56.765 | 1.00 | 58.81 |
| ATOM | 1328 | O | ASN | 179 | 17.108 | 14.553 | 57.441 | 1.00 | 59.10 |
| ATOM | 1329 | N | ASN | 180 | 16.860 | 16.599 | 56.537 | 1.00 | 58.30 |
| ATOM | 1330 | CA | ASN | 180 | 18.107 | 17.130 | 57.079 | 1.00 | 57.96 |
| ATOM | 1331 | CB | ASN | 180 | 18.362 | 18.539 | 56.539 | 1.00 | 58.57 |
| ATOM | 1332 | CG | ASN | 180 | 19.693 | 19.112 | 57.001 | 1.00 | 60.99 |
| ATOM | 1333 | OD1 | ASN | 180 | 20.278 | 18.647 | 57.983 | 1.00 | 60.88 |
| ATOM | 1334 | ND2 | ASN | 180 | 20.171 | 20.139 | 56.302 | 1.00 | 60.74 |
| ATOM | 1335 | C | ASN | 180 | 18.036 | 17.183 | 58.600 | 1.00 | 57.30 |
| ATOM | 1336 | O | ASN | 180 | 17.388 | 18.064 | 59.162 | 1.00 | 57.94 |
| ATOM | 1337 | N | VAL | 181 | 18.709 | 16.245 | 59.261 | 1.00 | 55.49 |
| ATOM | 1338 | CA | VAL | 181 | 18.716 | 16.189 | 60.720 | 1.00 | 54.19 |
| ATOM | 1339 | CB | VAL | 181 | 19.698 | 15.109 | 61.229 | 1.00 | 53.15 |
| ATOM | 1340 | CG1 | VAL | 181 | 19.756 | 15.121 | 62.748 | 1.00 | 50.90 |
| ATOM | 1341 | CG2 | VAL | 181 | 19.258 | 13.742 | 60.731 | 1.00 | 51.33 |
| ATOM | 1342 | C | VAL | 181 | 19.089 | 17.534 | 61.333 | 1.00 | 54.31 |
| ATOM | 1343 | O | VAL | 181 | 18.473 | 17.979 | 62.299 | 1.00 | 53.21 |
| ATOM | 1344 | N | VAL | 182 | 20.110 | 18.174 | 60.777 | 1.00 | 56.27 |
| ATOM | 1345 | CA | VAL | 182 | 20.533 | 19.472 | 61.271 | 1.00 | 58.32 |
| ATOM | 1346 | CB | VAL | 182 | 21.706 | 20.033 | 60.447 | 1.00 | 58.42 |
| ATOM | 1347 | CG1 | VAL | 182 | 22.135 | 21.373 | 61.007 | 1.00 | 58.05 |
| ATOM | 1348 | CG2 | VAL | 182 | 22.867 | 19.054 | 60.460 | 1.00 | 59.02 |
| ATOM | 1349 | C | VAL | 182 | 19.339 | 20.410 | 61.125 | 1.00 | 60.16 |
| ATOM | 1350 | O | VAL | 182 | 19.052 | 21.220 | 62.008 | 1.00 | 59.87 |
| ATOM | 1351 | N | GLY | 183 | 18.640 | 20.275 | 60.003 | 1.00 | 61.05 |
| ATOM | 1352 | CA | GLY | 183 | 17.480 | 21.103 | 59.741 | 1.00 | 63.47 |
| ATOM | 1353 | C | GLY | 183 | 16.412 | 20.967 | 60.805 | 1.00 | 64.68 |
| ATOM | 1354 | O | GLY | 183 | 15.873 | 21.966 | 61.280 | 1.00 | 64.59 |
| ATOM | 1355 | N | LEU | 184 | 16.103 | 19.733 | 61.187 | 1.00 | 65.39 |
| ATOM | 1356 | CA | LEU | 184 | 15.091 | 19.502 | 62.203 | 1.00 | 66.47 |
| ATOM | 1357 | CB | LEU | 184 | 14.855 | 18.005 | 62.387 | 1.00 | 66.17 |
| ATOM | 1358 | CG | LEU | 184 | 14.407 | 17.254 | 61.132 | 1.00 | 67.51 |
| ATOM | 1359 | CD1 | LEU | 184 | 14.116 | 15.805 | 61.486 | 1.00 | 66.63 |
| ATOM | 1360 | CD2 | LEU | 184 | 13.168 | 17.913 | 60.546 | 1.00 | 68.22 |
| ATOM | 1361 | C | LEU | 184 | 15.502 | 20.130 | 63.528 | 1.00 | 67.67 |
| ATOM | 1362 | O | LEU | 184 | 14.651 | 20.570 | 64.301 | 1.00 | 68.91 |
| ATOM | 1363 | N | LEU | 185 | 16.804 | 20.176 | 63.790 | 1.00 | 68.11 |
| ATOM | 1364 | CA | LEU | 185 | 17.297 | 20.759 | 65.031 | 1.00 | 68.91 |
| ATOM | 1365 | CB | LEU | 185 | 18.797 | 20.501 | 65.194 | 1.00 | 67.32 |
| ATOM | 1366 | CG | LEU | 185 | 19.409 | 21.060 | 66.482 | 1.00 | 66.21 |
| ATOM | 1367 | CD1 | LEU | 185 | 18.776 | 20.375 | 67.676 | 1.00 | 65.16 |
| ATOM | 1368 | CD2 | LEU | 185 | 20.913 | 20.851 | 66.486 | 1.00 | 66.43 |
| ATOM | 1369 | C | LEU | 185 | 17.034 | 22.262 | 65.058 | 1.00 | 70.10 |
| ATOM | 1370 | O | LEU | 185 | 16.422 | 22.776 | 65.991 | 1.00 | 70.26 |
| ATOM | 1371 | N | ARG | 186 | 17.505 | 22.962 | 64.033 | 1.00 | 71.83 |
| ATOM | 1372 | CA | ARG | 186 | 17.314 | 24.403 | 63.948 | 1.00 | 73.78 |
| ATOM | 1373 | CB | ARG | 186 | 18.015 | 24.941 | 62.700 | 1.00 | 73.97 |
| ATOM | 1374 | CG | ARG | 186 | 19.533 | 24.881 | 62.804 | 1.00 | 74.09 |
| ATOM | 1375 | CD | ARG | 186 | 20.206 | 24.984 | 61.448 | 1.00 | 74.37 |
| ATOM | 1376 | NE | ARG | 186 | 21.662 | 24.945 | 61.571 | 1.00 | 75.77 |
| ATOM | 1377 | CZ | ARG | 186 | 22.503 | 24.860 | 60.543 | 1.00 | 75.94 |
| ATOM | 1378 | NH1 | ARG | 186 | 22.036 | 24.800 | 59.303 | 1.00 | 75.97 |
| ATOM | 1379 | NH2 | ARG | 186 | 23.815 | 24.841 | 60.755 | 1.00 | 75.99 |
| ATOM | 1380 | C | ARG | 186 | 15.825 | 24.737 | 63.927 | 1.00 | 74.93 |
| ATOM | 1381 | O | ARG | 186 | 15.365 | 25.609 | 64.665 | 1.00 | 74.59 |
| ATOM | 1382 | N | ASP | 187 | 15.074 | 24.023 | 63.095 | 1.00 | 76.23 |
| ATOM | 1383 | CA | ASP | 187 | 13.632 | 24.225 | 62.981 | 1.00 | 77.59 |
| ATOM | 1384 | CB | ASP | 187 | 13.018 | 23.128 | 62.102 | 1.00 | 75.83 |
| ATOM | 1385 | CG | ASP | 187 | 13.203 | 23.391 | 60.614 | 1.00 | 74.87 |
| ATOM | 1386 | OD1 | ASP | 187 | 14.193 | 24.051 | 60.234 | 1.00 | 73.64 |
| ATOM | 1387 | OD2 | ASP | 187 | 12.359 | 22.924 | 59.820 | 1.00 | 74.33 |
| ATOM | 1388 | C | ASP | 187 | 12.945 | 24.236 | 64.349 | 1.00 | 79.78 |
| ATOM | 1389 | O | ASP | 187 | 11.963 | 24.952 | 64.551 | 1.00 | 80.50 |
| ATOM | 1390 | N | ALA | 188 | 13.461 | 23.445 | 65.286 | 1.00 | 81.21 |
| ATOM | 1391 | CA | ALA | 188 | 12.883 | 23.379 | 66.625 | 1.00 | 82.86 |
| ATOM | 1392 | CB | ALA | 188 | 13.118 | 22.000 | 67.230 | 1.00 | 83.00 |
| ATOM | 1393 | C | ALA | 188 | 13.477 | 24.456 | 67.525 | 1.00 | 84.14 |
| ATOM | 1394 | O | ALA | 188 | 12.783 | 25.019 | 68.376 | 1.00 | 84.10 |
| ATOM | 1395 | N | ILE | 189 | 14.763 | 24.736 | 67.338 | 1.00 | 85.33 |
| ATOM | 1396 | CA | ILE | 189 | 15.445 | 25.753 | 68.127 | 1.00 | 86.87 |
| ATOM | 1397 | CB | ILE | 189 | 16.947 | 25.819 | 67.776 | 1.00 | 86.40 |
| ATOM | 1398 | CG2 | ILE | 189 | 17.585 | 27.049 | 68.409 | 1.00 | 85.37 |
| ATOM | 1399 | CG1 | ILE | 189 | 17.641 | 24.541 | 68.253 | 1.00 | 86.52 |
| ATOM | 1400 | CD1 | ILE | 189 | 19.136 | 24.516 | 68.004 | 1.00 | 86.36 |
| ATOM | 1401 | C | ILE | 189 | 14.812 | 27.114 | 67.871 | 1.00 | 88.85 |
| ATOM | 1402 | O | ILE | 189 | 14.802 | 27.978 | 68.748 | 1.00 | 89.64 |
| ATOM | 1403 | N | LYS | 190 | 14.278 | 27.295 | 66.666 | 1.00 | 90.36 |
| ATOM | 1404 | CA | LYS | 190 | 13.638 | 28.551 | 66.291 | 1.00 | 91.75 |
| ATOM | 1405 | CB | LYS | 190 | 13.678 | 28.729 | 64.770 | 1.00 | 92.26 |
| ATOM | 1406 | CG | LYS | 190 | 15.032 | 29.205 | 64.234 | 1.00 | 93.56 |
| ATOM | 1407 | CD | LYS | 190 | 16.174 | 28.282 | 64.652 | 1.00 | 94.17 |
| ATOM | 1408 | CE | LYS | 190 | 17.507 | 28.722 | 64.064 | 1.00 | 94.42 |
| ATOM | 1409 | NZ | LYS | 190 | 18.605 | 27.773 | 64.409 | 1.00 | 93.91 |
| ATOM | 1410 | C | LYS | 190 | 12.202 | 28.645 | 66.803 | 1.00 | 92.34 |
| ATOM | 1411 | O | LYS | 190 | 11.612 | 29.723 | 66.817 | 1.00 | 92.82 |
| ATOM | 1412 | N | ARG | 191 | 11.639 | 27.516 | 67.221 | 1.00 | 92.78 |
| ATOM | 1413 | CA | ARG | 191 | 10.286 | 27.502 | 67.763 | 1.00 | 93.41 |
| ATOM | 1414 | CB | ARG | 191 | 9.674 | 26.108 | 67.658 | 1.00 | 93.77 |
| ATOM | 1415 | CG | ARG | 191 | 9.711 | 25.497 | 66.275 | 1.00 | 93.66 |
| ATOM | 1416 | CD | ARG | 191 | 9.530 | 23.993 | 66.378 | 1.00 | 93.81 |
| ATOM | 1417 | NE | ARG | 191 | 9.816 | 23.310 | 65.123 | 1.00 | 93.99 |
| ATOM | 1418 | CZ | ARG | 191 | 10.012 | 22.000 | 65.017 | 1.00 | 94.26 |
| ATOM | 1419 | NH1 | ARG | 191 | 9.954 | 21.231 | 66.095 | 1.00 | 94.65 |
| ATOM | 1420 | NH2 | ARG | 191 | 10.269 | 21.459 | 63.835 | 1.00 | 94.96 |
| ATOM | 1421 | C | ARG | 191 | 10.432 | 27.866 | 69.233 | 1.00 | 94.00 |
| ATOM | 1422 | O | ARG | 191 | 9.526 | 27.654 | 70.036 | 1.00 | 94.16 |
| ATOM | 1423 | N | ARG | 192 | 11.596 | 28.408 | 69.574 | 1.00 | 94.91 |
| ATOM | 1424 | CA | ARG | 192 | 11.897 | 28.795 | 70.943 | 1.00 | 96.15 |
| ATOM | 1425 | CB | ARG | 192 | 13.049 | 27.944 | 71.482 | 1.00 | 96.57 |
| ATOM | 1426 | CG | ARG | 192 | 12.733 | 26.469 | 71.581 | 1.00 | 97.44 |
| ATOM | 1427 | CD | ARG | 192 | 11.737 | 26.209 | 72.689 | 1.00 | 98.38 |
| ATOM | 1428 | NE | ARG | 192 | 11.339 | 24.808 | 72.745 | 1.00 | 98.97 |
| ATOM | 1429 | CZ | ARG | 192 | 10.624 | 24.278 | 73.730 | 1.00 | 99.63 |
| ATOM | 1430 | NH1 | ARG | 192 | 10.230 | 25.035 | 74.746 | 1.00 | 99.17 |
| ATOM | 1431 | NH2 | ARG | 192 | 10.300 | 22.992 | 73.700 | 1.00 | 99.97 |
| ATOM | 1432 | C | ARG | 192 | 12.273 | 30.265 | 71.062 | 1.00 | 96.50 |
| ATOM | 1433 | O | ARG | 192 | 11.603 | 31.035 | 71.752 | 1.00 | 96.60 |
| ATOM | 1434 | N | GLY | 193 | 13.352 | 30.652 | 70.386 | 1.00 | 96.89 |
| ATOM | 1435 | CA | GLY | 193 | 13.812 | 32.026 | 70.465 | 1.00 | 97.08 |
| ATOM | 1436 | C | GLY | 193 | 14.385 | 32.217 | 71.855 | 1.00 | 97.08 |
| ATOM | 1437 | O | GLY | 193 | 15.060 | 33.204 | 72.147 | 1.00 | 96.46 |
| ATOM | 1438 | N | ASP | 194 | 14.104 | 31.235 | 72.707 | 1.00 | 97.36 |
| ATOM | 1439 | CA | ASP | 194 | 14.552 | 31.205 | 74.092 | 1.00 | 97.35 |
| ATOM | 1440 | CB | ASP | 194 | 13.938 | 29.984 | 74.789 | 1.00 | 98.43 |
| ATOM | 1441 | CG | ASP | 194 | 13.764 | 30.181 | 76.284 | 1.00 | 99.62 |
| ATOM | 1442 | OD1 | ASP | 194 | 13.117 | 31.173 | 76.683 | 1.00 | 100.29 |
| ATOM | 1443 | OD2 | ASP | 194 | 14.262 | 29.338 | 77.059 | 1.00 | 99.87 |
| ATOM | 1444 | C | ASP | 194 | 16.078 | 31.122 | 74.122 | 1.00 | 96.90 |
| ATOM | 1445 | O | ASP | 194 | 16.715 | 31.471 | 75.118 | 1.00 | 97.50 |
| ATOM | 1446 | N | PHE | 195 | 16.657 | 30.655 | 73.018 | 1.00 | 95.62 |
| ATOM | 1447 | CA | PHE | 195 | 18.105 | 30.524 | 72.896 | 1.00 | 94.15 |
| ATOM | 1448 | CB | PHE | 195 | 18.598 | 29.309 | 73.697 | 1.00 | 94.86 |
| ATOM | 1449 | CG | PHE | 195 | 18.043 | 27.988 | 73.224 | 1.00 | 95.10 |
| ATOM | 1450 | CD1 | PHE | 195 | 18.560 | 27.360 | 72.095 | 1.00 | 95.09 |
| ATOM | 1451 | CD2 | PHE | 195 | 17.005 | 27.369 | 73.916 | 1.00 | 95.17 |
| ATOM | 1452 | CE1 | PHE | 195 | 18.053 | 26.136 | 71.663 | 1.00 | 95.14 |
| ATOM | 1453 | CE2 | PHE | 195 | 16.491 | 26.145 | 73.492 | 1.00 | 95.12 |
| ATOM | 1454 | CZ | PHE | 195 | 17.016 | 25.528 | 72.364 | 1.00 | 94.97 |
| ATOM | 1455 | C | PHE | 195 | 18.508 | 30.393 | 71.430 | 1.00 | 92.64 |
| ATOM | 1456 | O | PHE | 195 | 17.667 | 30.131 | 70.569 | 1.00 | 92.22 |
| ATOM | 1457 | N | GLU | 196 | 19.793 | 30.583 | 71.148 | 1.00 | 90.93 |
| ATOM | 1458 | CA | GLU | 196 | 20.292 | 30.486 | 69.779 | 1.00 | 89.04 |
| ATOM | 1459 | CB | GLU | 196 | 20.249 | 31.861 | 69.102 | 1.00 | 89.55 |
| ATOM | 1460 | CG | GLU | 196 | 18.846 | 32.395 | 68.832 | 1.00 | 90.25 |
| ATOM | 1461 | CD | GLU | 196 | 18.859 | 33.771 | 68.187 | 1.00 | 90.61 |
| ATOM | 1462 | OE1 | GLU | 196 | 19.342 | 34.728 | 68.830 | 1.00 | 90.30 |
| ATOM | 1463 | OE2 | GLU | 196 | 18.390 | 33.895 | 67.035 | 1.00 | 90.79 |
| ATOM | 1464 | C | GLU | 196 | 21.711 | 29.923 | 69.694 | 1.00 | 87.11 |
| ATOM | 1465 | O | GLU | 196 | 22.681 | 30.589 | 70.066 | 1.00 | 86.83 |
| ATOM | 1466 | N | MET | 197 | 21.824 | 28.692 | 69.201 | 1.00 | 84.23 |
| ATOM | 1467 | CA | MET | 197 | 23.121 | 28.043 | 69.045 | 1.00 | 80.79 |
| ATOM | 1468 | CB | MET | 197 | 23.067 | 26.586 | 69.524 | 1.00 | 81.16 |
| ATOM | 1469 | CG | MET | 197 | 22.633 | 26.389 | 70.967 | 1.00 | 80.14 |
| ATOM | 1470 | SD | MET | 197 | 23.597 | 27.356 | 72.135 | 1.00 | 81.93 |
| ATOM | 1471 | CE | MET | 197 | 25.195 | 26.640 | 71.968 | 1.00 | 81.72 |
| ATOM | 1472 | C | MET | 197 | 23.502 | 28.070 | 67.568 | 1.00 | 77.74 |
| ATOM | 1473 | O | MET | 197 | 22.695 | 28.436 | 66.716 | 1.00 | 76.30 |
| ATOM | 1474 | N | ASP | 198 | 24.733 | 27.672 | 67.269 | 1.00 | 75.73 |
| ATOM | 1475 | CA | ASP | 198 | 25.214 | 27.652 | 65.894 | 1.00 | 72.50 |
| ATOM | 1476 | CB | ASP | 198 | 26.297 | 28.723 | 65.720 | 1.00 | 73.47 |
| ATOM | 1477 | CG | ASP | 198 | 26.573 | 29.046 | 64.265 | 1.00 | 75.34 |
| ATOM | 1478 | OD1 | ASP | 198 | 27.407 | 29.941 | 64.005 | 1.00 | 75.72 |
| ATOM | 1479 | OD2 | ASP | 198 | 25.954 | 28.407 | 63.384 | 1.00 | 76.53 |
| ATOM | 1480 | C | ASP | 198 | 25.769 | 26.265 | 65.544 | 1.00 | 69.75 |
| ATOM | 1481 | O | ASP | 198 | 26.962 | 26.005 | 65.703 | 1.00 | 69.48 |
| ATOM | 1482 | N | VAL | 199 | 24.892 | 25.383 | 65.068 | 1.00 | 66.43 |
| ATOM | 1483 | CA | VAL | 199 | 25.266 | 24.018 | 64.697 | 1.00 | 62.47 |
| ATOM | 1484 | CB | VAL | 199 | 24.055 | 23.266 | 64.113 | 1.00 | 62.19 |
| ATOM | 1485 | CG1 | VAL | 199 | 24.426 | 21.823 | 63.827 | 1.00 | 61.88 |
| ATOM | 1486 | CG2 | VAL | 199 | 22.886 | 23.340 | 65.083 | 1.00 | 61.21 |
| ATOM | 1487 | C | VAL | 199 | 26.409 | 23.986 | 63.682 | 1.00 | 60.44 |
| ATOM | 1488 | O | VAL | 199 | 26.192 | 24.135 | 62.479 | 1.00 | 59.37 |
| ATOM | 1489 | N | VAL | 200 | 27.624 | 23.774 | 64.180 | 1.00 | 58.00 |
| ATOM | 1490 | CA | VAL | 200 | 28.820 | 23.741 | 63.341 | 1.00 | 56.24 |
| ATOM | 1491 | CB | VAL | 200 | 30.048 | 24.278 | 64.128 | 1.00 | 57.42 |
| ATOM | 1492 | CG1 | VAL | 200 | 31.326 | 24.071 | 63.331 | 1.00 | 57.94 |
| ATOM | 1493 | CG2 | VAL | 200 | 29.859 | 25.761 | 64.433 | 1.00 | 59.29 |
| ATOM | 1494 | C | VAL | 200 | 29.159 | 22.357 | 62.785 | 1.00 | 54.53 |
| ATOM | 1495 | O | VAL | 200 | 29.759 | 22.242 | 61.715 | 1.00 | 54.29 |
| ATOM | 1496 | N | ALA | 201 | 28.779 | 21.306 | 63.503 | 1.00 | 52.88 |
| ATOM | 1497 | CA | ALA | 201 | 29.085 | 19.953 | 63.048 | 1.00 | 49.83 |
| ATOM | 1498 | CB | ALA | 201 | 30.541 | 19.627 | 63.349 | 1.00 | 47.49 |
| ATOM | 1499 | C | ALA | 201 | 28.196 | 18.887 | 63.654 | 1.00 | 46.62 |
| ATOM | 1500 | O | ALA | 201 | 27.803 | 18.973 | 64.810 | 1.00 | 47.96 |
| ATOM | 1501 | N | MET | 202 | 27.873 | 17.883 | 62.851 | 1.00 | 44.82 |
| ATOM | 1502 | CA | MET | 202 | 27.065 | 16.762 | 63.309 | 1.00 | 42.12 |
| ATOM | 1503 | CB | MET | 202 | 25.731 | 16.695 | 62.567 | 1.00 | 39.23 |
| ATOM | 1504 | CG | MET | 202 | 24.886 | 15.520 | 63.014 | 1.00 | 38.13 |
| ATOM | 1505 | SD | MET | 202 | 23.425 | 15.193 | 62.026 | 1.00 | 40.98 |
| ATOM | 1506 | CE | MET | 202 | 24.134 | 15.162 | 60.401 | 1.00 | 37.63 |
| ATOM | 1507 | C | MET | 202 | 27.865 | 15.489 | 63.027 | 1.00 | 40.68 |
| ATOM | 1508 | O | MET | 202 | 28.274 | 15.251 | 61.888 | 1.00 | 38.74 |
| ATOM | 1509 | N | VAL | 203 | 28.092 | 14.679 | 64.060 | 1.00 | 39.90 |
| ATOM | 1510 | CA | VAL | 203 | 28.851 | 13.438 | 63.901 | 1.00 | 37.47 |
| ATOM | 1511 | CB | VAL | 203 | 30.264 | 13.549 | 64.517 | 1.00 | 36.73 |
| ATOM | 1512 | CG1 | VAL | 203 | 31.078 | 14.615 | 63.796 | 1.00 | 34.96 |
| ATOM | 1513 | CG2 | VAL | 203 | 30.155 | 13.852 | 65.996 | 1.00 | 37.90 |
| ATOM | 1514 | C | VAL | 203 | 28.190 | 12.199 | 64.505 | 1.00 | 37.09 |
| ATOM | 1515 | O | VAL | 203 | 27.250 | 12.284 | 65.309 | 1.00 | 36.61 |
| ATOM | 1516 | N | ASN | 204 | 28.707 | 11.039 | 64.101 | 1.00 | 36.09 |
| ATOM | 1517 | CA | ASN | 204 | 28.228 | 9.749 | 64.584 | 1.00 | 31.60 |
| ATOM | 1518 | CB | ASN | 204 | 28.461 | 8.695 | 63.497 | 1.00 | 32.07 |
| ATOM | 1519 | CG | ASN | 204 | 27.949 | 7.322 | 63.888 | 1.00 | 31.63 |
| ATOM | 1520 | OD1 | ASN | 204 | 28.729 | 6.443 | 64.250 | 1.00 | 30.91 |
| ATOM | 1521 | ND2 | ASN | 204 | 26.634 | 7.135 | 63.824 | 1.00 | 28.99 |
| ATOM | 1522 | C | ASN | 204 | 29.027 | 9.454 | 65.853 | 1.00 | 28.81 |
| ATOM | 1523 | O | ASN | 204 | 30.122 | 9.990 | 66.019 | 1.00 | 30.24 |
| ATOM | 1524 | N | ASP | 205 | 28.498 | 8.639 | 66.765 | 1.00 | 27.90 |
| ATOM | 1525 | CA | ASP | 205 | 29.240 | 8.361 | 67.995 | 1.00 | 26.70 |
| ATOM | 1526 | CB | ASP | 205 | 28.369 | 7.627 | 69.028 | 1.00 | 27.65 |
| ATOM | 1527 | CG | ASP | 205 | 27.642 | 6.438 | 68.455 | 1.00 | 30.26 |
| ATOM | 1528 | OD1 | ASP | 205 | 27.079 | 5.655 | 69.256 | 1.00 | 28.60 |
| ATOM | 1529 | OD2 | ASP | 205 | 27.623 | 6.289 | 67.213 | 1.00 | 31.99 |
| ATOM | 1530 | C | ASP | 205 | 30.573 | 7.630 | 67.791 | 1.00 | 26.87 |
| ATOM | 1531 | O | ASP | 205 | 31.498 | 7.810 | 68.581 | 1.00 | 27.79 |
| ATOM | 1532 | N | THR | 206 | 30.686 | 6.816 | 66.740 | 1.00 | 24.79 |
| ATOM | 1533 | CA | THR | 206 | 31.951 | 6.146 | 66.476 | 1.00 | 24.03 |
| ATOM | 1534 | CB | THR | 206 | 31.886 | 5.236 | 65.206 | 1.00 | 25.43 |
| ATOM | 1535 | OG1 | THR | 206 | 31.401 | 5.999 | 64.089 | 1.00 | 26.30 |
| ATOM | 1536 | CG2 | THR | 206 | 30.976 | 4.032 | 65.444 | 1.00 | 16.83 |
| ATOM | 1537 | C | THR | 206 | 32.970 | 7.258 | 66.220 | 1.00 | 24.75 |
| ATOM | 1538 | O | THR | 206 | 34.025 | 7.326 | 66.858 | 1.00 | 25.65 |
| ATOM | 1539 | N | VAL | 207 | 32.632 | 8.136 | 65.285 | 1.00 | 22.49 |
| ATOM | 1540 | CA | VAL | 207 | 33.487 | 9.257 | 64.917 | 1.00 | 23.51 |
| ATOM | 1541 | CB | VAL | 207 | 32.775 | 10.133 | 63.855 | 1.00 | 25.35 |
| ATOM | 1542 | CG1 | VAL | 207 | 33.617 | 11.362 | 63.521 | 1.00 | 24.61 |
| ATOM | 1543 | CG2 | VAL | 207 | 32.509 | 9.299 | 62.609 | 1.00 | 21.66 |
| ATOM | 1544 | C | VAL | 207 | 33.897 | 10.119 | 66.126 | 1.00 | 23.48 |
| ATOM | 1545 | O | VAL | 207 | 35.061 | 10.470 | 66.279 | 1.00 | 26.51 |
| ATOM | 1546 | N | ALA | 208 | 32.948 | 10.452 | 66.989 | 1.00 | 24.53 |
| ATOM | 1547 | CA | ALA | 208 | 33.262 | 11.251 | 68.169 | 1.00 | 26.32 |
| ATOM | 1548 | CB | ALA | 208 | 31.980 | 11.533 | 68.958 | 1.00 | 27.56 |
| ATOM | 1549 | C | ALA | 208 | 34.287 | 10.530 | 69.055 | 1.00 | 28.84 |
| ATOM | 1550 | O | ALA | 208 | 35.247 | 11.138 | 69.549 | 1.00 | 27.69 |
| ATOM | 1551 | N | THR | 209 | 34.084 | 9.228 | 69.258 | 1.00 | 28.76 |
| ATOM | 1552 | CA | THR | 209 | 35.006 | 8.447 | 70.075 | 1.00 | 28.08 |
| ATOM | 1553 | CB | THR | 209 | 34.474 | 7.001 | 70.271 | 1.00 | 31.76 |
| ATOM | 1554 | OG1 | THR | 209 | 33.373 | 7.027 | 71.181 | 1.00 | 33.12 |
| ATOM | 1555 | CG2 | THR | 209 | 35.550 | 6.080 | 70.818 | 1.00 | 30.03 |
| ATOM | 1556 | C | THR | 209 | 36.382 | 8.414 | 69.418 | 1.00 | 26.73 |
| ATOM | 1557 | O | THR | 209 | 37.399 | 8.611 | 70.078 | 1.00 | 28.00 |
| ATOM | 1558 | N | MET | 210 | 36.421 | 8.191 | 68.110 | 1.00 | 28.44 |
| ATOM | 1559 | CA | MET | 210 | 37.703 | 8.143 | 67.419 | 1.00 | 28.08 |
| ATOM | 1560 | CB | MET | 210 | 37.516 | 7.851 | 65.932 | 1.00 | 26.94 |
| ATOM | 1561 | CG | MET | 210 | 38.842 | 7.766 | 65.168 | 1.00 | 28.59 |
| ATOM | 1562 | SD | MET | 210 | 38.643 | 7.734 | 63.374 | 1.00 | 32.14 |
| ATOM | 1563 | CE | MET | 210 | 38.216 | 9.518 | 63.083 | 1.00 | 33.30 |
| ATOM | 1564 | C | MET | 210 | 38.467 | 9.452 | 67.578 | 1.00 | 29.43 |
| ATOM | 1565 | O | MET | 210 | 39.636 | 9.459 | 67.972 | 1.00 | 30.57 |
| ATOM | 1566 | N | ILE | 211 | 37.799 | 10.561 | 67.281 | 1.00 | 31.16 |
| ATOM | 1567 | CA | ILE | 211 | 38.433 | 11.873 | 67.376 | 1.00 | 30.57 |
| ATOM | 1568 | CB | ILE | 211 | 37.418 | 13.012 | 67.019 | 1.00 | 29.75 |
| ATOM | 1569 | CG2 | ILE | 211 | 38.086 | 14.390 | 67.177 | 1.00 | 28.08 |
| ATOM | 1570 | CG1 | ILE | 211 | 36.928 | 12.837 | 65.578 | 1.00 | 22.83 |
| ATOM | 1571 | CD1 | ILE | 211 | 38.021 | 12.979 | 64.553 | 1.00 | 26.28 |
| ATOM | 1572 | C | ILE | 211 | 39.014 | 12.128 | 68.762 | 1.00 | 30.30 |
| ATOM | 1573 | O | ILE | 211 | 40.185 | 12.489 | 68.897 | 1.00 | 31.89 |
| ATOM | 1574 | N | SER | 212 | 38.203 | 11.914 | 69.792 | 1.00 | 32.78 |
| ATOM | 1575 | CA | SER | 212 | 38.639 | 12.146 | 71.164 | 1.00 | 35.84 |
| ATOM | 1576 | CB | SER | 212 | 37.499 | 11.852 | 72.140 | 1.00 | 35.91 |
| ATOM | 1577 | OG | SER | 212 | 37.317 | 10.455 | 72.307 | 1.00 | 41.55 |
| ATOM | 1578 | C | SER | 212 | 39.864 | 11.334 | 71.566 | 1.00 | 37.74 |
| ATOM | 1579 | O | SER | 212 | 40.684 | 11.803 | 72.354 | 1.00 | 41.44 |
| ATOM | 1580 | N | CYS | 213 | 39.990 | 10.121 | 71.040 | 1.00 | 38.07 |
| ATOM | 1581 | CA | CYS | 213 | 41.132 | 9.273 | 71.374 | 1.00 | 39.83 |
| ATOM | 1582 | CB | CYS | 213 | 40.802 | 7.799 | 71.108 | 1.00 | 38.31 |
| ATOM | 1583 | SG | CYS | 213 | 39.513 | 7.129 | 72.185 | 1.00 | 38.48 |
| ATOM | 1584 | C | CYS | 213 | 42.372 | 9.666 | 70.582 | 1.00 | 41.86 |
| ATOM | 1585 | O | CYS | 213 | 43.503 | 9.426 | 71.012 | 1.00 | 38.47 |
| ATOM | 1586 | N | TYR | 214 | 42.149 | 10.261 | 69.413 | 1.00 | 45.32 |
| ATOM | 1587 | CA | TYR | 214 | 43.243 | 10.701 | 68.554 | 1.00 | 45.02 |
| ATOM | 1588 | CB | TYR | 214 | 42.705 | 11.506 | 67.370 | 1.00 | 45.88 |
| ATOM | 1589 | CG | TYR | 214 | 43.798 | 12.171 | 66.573 | 1.00 | 45.72 |
| ATOM | 1590 | CD1 | TYR | 214 | 44.509 | 11.465 | 65.608 | 1.00 | 46.39 |
| ATOM | 1591 | CE1 | TYR | 214 | 45.556 | 12.061 | 64.913 | 1.00 | 47.16 |
| ATOM | 1592 | CD2 | TYR | 214 | 44.160 | 13.498 | 66.823 | 1.00 | 44.53 |
| ATOM | 1593 | CE2 | TYR | 214 | 45.203 | 14.099 | 66.134 | 1.00 | 45.20 |
| ATOM | 1594 | CZ | TYR | 214 | 45.896 | 13.375 | 65.183 | 1.00 | 46.22 |
| ATOM | 1595 | OH | TYR | 214 | 46.942 | 13.955 | 64.510 | 1.00 | 50.31 |
| ATOM | 1596 | C | TYR | 214 | 44.226 | 11.573 | 69.322 | 1.00 | 44.69 |
| ATOM | 1597 | O | TYR | 214 | 45.420 | 11.296 | 69.363 | 1.00 | 44.40 |
| ATOM | 1598 | N | TYR | 215 | 43.713 | 12.635 | 69.924 | 1.00 | 45.92 |
| ATOM | 1599 | CA | TYR | 215 | 44.556 | 13.552 | 70.667 | 1.00 | 48.38 |
| ATOM | 1600 | CB | TYR | 215 | 43.713 | 14.716 | 71.175 | 1.00 | 51.93 |
| ATOM | 1601 | CG | TYR | 215 | 43.192 | 15.545 | 70.021 | 1.00 | 57.70 |
| ATOM | 1602 | CD1 | TYR | 215 | 41.918 | 15.330 | 69.484 | 1.00 | 58.41 |
| ATOM | 1603 | CE1 | TYR | 215 | 41.478 | 16.047 | 68.363 | 1.00 | 61.27 |
| ATOM | 1604 | CD2 | TYR | 215 | 44.011 | 16.498 | 69.413 | 1.00 | 59.13 |
| ATOM | 1605 | CE2 | TYR | 215 | 43.586 | 17.214 | 68.300 | 1.00 | 61.22 |
| ATOM | 1606 | CZ | TYR | 215 | 42.325 | 16.991 | 67.780 | 1.00 | 62.20 |
| ATOM | 1607 | OH | TYR | 215 | 41.928 | 17.728 | 66.688 | 1.00 | 61.67 |
| ATOM | 1608 | C | TYR | 215 | 45.304 | 12.871 | 71.792 | 1.00 | 48.87 |
| ATOM | 1609 | O | TYR | 215 | 46.282 | 13.407 | 72.318 | 1.00 | 49.38 |
| ATOM | 1610 | N | GLU | 216 | 44.852 | 11.672 | 72.142 | 1.00 | 47.69 |
| ATOM | 1611 | CA | GLU | 216 | 45.496 | 10.889 | 73.181 | 1.00 | 47.03 |
| ATOM | 1612 | CB | GLU | 216 | 44.474 | 9.979 | 73.863 | 1.00 | 49.83 |
| ATOM | 1613 | CG | GLU | 216 | 44.837 | 9.550 | 75.270 | 1.00 | 55.37 |
| ATOM | 1614 | CD | GLU | 216 | 44.998 | 10.735 | 76.208 | 1.00 | 59.31 |
| ATOM | 1615 | OE1 | GLU | 216 | 44.285 | 11.747 | 76.012 | 1.00 | 59.95 |
| ATOM | 1616 | OE2 | GLU | 216 | 45.824 | 10.649 | 77.146 | 1.00 | 60.13 |
| ATOM | 1617 | C | GLU | 216 | 46.552 | 10.044 | 72.477 | 1.00 | 45.45 |
| ATOM | 1618 | O | GLU | 216 | 47.673 | 9.905 | 72.958 | 1.00 | 45.05 |
| ATOM | 1619 | N | ASP | 217 | 46.183 | 9.495 | 71.321 | 1.00 | 43.73 |
| ATOM | 1620 | CA | ASP | 217 | 47.074 | 8.643 | 70.530 | 1.00 | 41.33 |
| ATOM | 1621 | CB | ASP | 217 | 46.776 | 7.171 | 70.845 | 1.00 | 40.13 |
| ATOM | 1622 | CG | ASP | 217 | 47.780 | 6.208 | 70.226 | 1.00 | 39.76 |
| ATOM | 1623 | OD1 | ASP | 217 | 48.461 | 6.571 | 69.249 | 1.00 | 40.95 |
| ATOM | 1624 | OD2 | ASP | 217 | 47.876 | 5.062 | 70.712 | 1.00 | 42.61 |
| ATOM | 1625 | C | ASP | 217 | 46.852 | 8.921 | 69.033 | 1.00 | 40.53 |
| ATOM | 1626 | O | ASP | 217 | 45.862 | 8.474 | 68.443 | 1.00 | 37.20 |
| ATOM | 1627 | N | HIS | 218 | 47.779 | 9.657 | 68.427 | 1.00 | 41.94 |
| ATOM | 1628 | CA | HIS | 218 | 47.689 | 10.008 | 67.007 | 1.00 | 44.23 |
| ATOM | 1629 | CB | HIS | 218 | 48.912 | 10.828 | 66.603 | 1.00 | 47.00 |
| ATOM | 1630 | CG | HIS | 218 | 48.860 | 12.244 | 67.079 | 1.00 | 51.95 |
| ATOM | 1631 | CD2 | HIS | 218 | 49.230 | 13.402 | 66.483 | 1.00 | 54.47 |
| ATOM | 1632 | ND1 | HIS | 218 | 48.371 | 12.592 | 68.320 | 1.00 | 54.33 |
| ATOM | 1633 | CE1 | HIS | 218 | 48.439 | 13.903 | 68.467 | 1.00 | 55.83 |
| ATOM | 1634 | NE2 | HIS | 218 | 48.957 | 14.419 | 67.367 | 1.00 | 55.95 |
| ATOM | 1635 | C | HIS | 218 | 47.528 | 8.810 | 66.074 | 1.00 | 42.66 |
| ATOM | 1636 | O | HIS | 218 | 47.157 | 8.963 | 64.909 | 1.00 | 42.00 |
| ATOM | 1637 | N | GLN | 219 | 47.793 | 7.620 | 66.597 | 1.00 | 41.40 |
| ATOM | 1638 | CA | GLN | 219 | 47.667 | 6.394 | 65.820 | 1.00 | 41.15 |
| ATOM | 1639 | CB | GLN | 219 | 48.592 | 5.321 | 66.397 | 1.00 | 45.16 |
| ATOM | 1640 | CG | GLN | 219 | 50.070 | 5.611 | 66.214 | 1.00 | 49.72 |
| ATOM | 1641 | CD | GLN | 219 | 50.566 | 5.230 | 64.832 | 1.00 | 55.92 |
| ATOM | 1642 | OE1 | GLN | 219 | 49.997 | 5.646 | 63.813 | 1.00 | 57.28 |
| ATOM | 1643 | NE2 | GLN | 219 | 51.636 | 4.429 | 64.787 | 1.00 | 57.32 |
| ATOM | 1644 | C | GLN | 219 | 46.228 | 5.869 | 65.792 | 1.00 | 37.41 |
| ATOM | 1645 | O | GLN | 219 | 45.927 | 4.904 | 65.091 | 1.00 | 37.06 |
| ATOM | 1646 | N | CYS | 220 | 45.342 | 6.488 | 66.562 | 1.00 | 34.18 |
| ATOM | 1647 | CA | CYS | 220 | 43.955 | 6.038 | 66.578 | 1.00 | 32.52 |
| ATOM | 1648 | CB | CYS | 220 | 43.199 | 6.597 | 67.783 | 1.00 | 28.93 |
| ATOM | 1649 | SG | CYS | 220 | 41.420 | 6.288 | 67.739 | 1.00 | 31.90 |
| ATOM | 1650 | C | CYS | 220 | 43.272 | 6.474 | 65.303 | 1.00 | 32.01 |
| ATOM | 1651 | O | CYS | 220 | 43.010 | 7.664 | 65.096 | 1.00 | 32.91 |
| ATOM | 1652 | N | GLU | 221 | 42.993 | 5.505 | 64.442 | 1.00 | 29.12 |
| ATOM | 1653 | CA | GLU | 221 | 42.343 | 5.785 | 63.176 | 1.00 | 28.98 |
| ATOM | 1654 | CB | GLU | 221 | 43.273 | 5.437 | 62.009 | 1.00 | 30.00 |
| ATOM | 1655 | CG | GLU | 221 | 44.481 | 6.366 | 61.853 | 1.00 | 35.29 |
| ATOM | 1656 | CD | GLU | 221 | 45.190 | 6.166 | 60.515 | 1.00 | 36.83 |
| ATOM | 1657 | OE1 | GLU | 221 | 44.490 | 6.007 | 59.498 | 1.00 | 38.09 |
| ATOM | 1658 | OE2 | GLU | 221 | 46.436 | 6.176 | 60.465 | 1.00 | 40.80 |
| ATOM | 1659 | C | GLU | 221 | 41.057 | 4.991 | 63.059 | 1.00 | 25.46 |
| ATOM | 1660 | O | GLU | 221 | 40.513 | 4.835 | 61.970 | 1.00 | 22.65 |
| ATOM | 1661 | N | VAL | 222 | 40.569 | 4.491 | 64.185 | 1.00 | 25.43 |
| ATOM | 1662 | CA | VAL | 222 | 39.337 | 3.703 | 64.179 | 1.00 | 25.45 |
| ATOM | 1663 | CB | VAL | 222 | 39.625 | 2.172 | 64.189 | 1.00 | 24.36 |
| ATOM | 1664 | CG1 | VAL | 222 | 38.318 | 1.391 | 64.122 | 1.00 | 21.56 |
| ATOM | 1665 | CG2 | VAL | 222 | 40.533 | 1.795 | 63.029 | 1.00 | 21.70 |
| ATOM | 1666 | C | VAL | 222 | 38.527 | 4.016 | 65.414 | 1.00 | 25.44 |
| ATOM | 1667 | O | VAL | 222 | 39.076 | 4.192 | 66.492 | 1.00 | 25.99 |
| ATOM | 1668 | N | GLY | 223 | 37.217 | 4.090 | 65.240 | 1.00 | 25.97 |
| ATOM | 1669 | CA | GLY | 223 | 36.328 | 4.347 | 66.349 | 1.00 | 25.83 |
| ATOM | 1670 | C | GLY | 223 | 35.337 | 3.201 | 66.340 | 1.00 | 25.37 |
| ATOM | 1671 | O | GLY | 223 | 34.852 | 2.812 | 65.273 | 1.00 | 25.38 |
| ATOM | 1672 | N | MET | 224 | 35.044 | 2.647 | 67.511 | 1.00 | 24.88 |
| ATOM | 1673 | CA | MET | 224 | 34.114 | 1.527 | 67.587 | 1.00 | 25.47 |
| ATOM | 1674 | CB | MET | 224 | 34.881 | 0.187 | 67.638 | 1.00 | 22.66 |
| ATOM | 1675 | CG | MET | 224 | 33.956 | −1.041 | 67.634 | 1.00 | 25.14 |
| ATOM | 1676 | SD | MET | 224 | 34.806 | −2.680 | 67.748 | 1.00 | 22.18 |
| ATOM | 1677 | CE | MET | 224 | 35.380 | −2.594 | 69.396 | 1.00 | 16.01 |
| ATOM | 1678 | C | MET | 224 | 33.177 | 1.618 | 68.780 | 1.00 | 22.20 |
| ATOM | 1679 | O | MET | 224 | 33.577 | 1.978 | 69.881 | 1.00 | 22.65 |
| ATOM | 1680 | N | ILE | 225 | 31.915 | 1.295 | 68.543 | 1.00 | 21.12 |
| ATOM | 1681 | CA | ILE | 225 | 30.936 | 1.314 | 69.604 | 1.00 | 21.34 |
| ATOM | 1682 | CB | ILE | 225 | 29.757 | 2.295 | 69.293 | 1.00 | 25.85 |
| ATOM | 1683 | CG2 | ILE | 225 | 28.739 | 2.268 | 70.446 | 1.00 | 25.47 |
| ATOM | 1684 | CG1 | ILE | 225 | 30.273 | 3.734 | 69.107 | 1.00 | 25.08 |
| ATOM | 1685 | CD1 | ILE | 225 | 30.838 | 4.355 | 70.382 | 1.00 | 22.09 |
| ATOM | 1686 | C | ILE | 225 | 30.321 | −0.080 | 69.789 | 1.00 | 22.30 |
| ATOM | 1687 | O | ILE | 225 | 29.885 | −0.712 | 68.826 | 1.00 | 24.03 |
| ATOM | 1688 | N | VAL | 226 | 30.313 | −0.563 | 71.025 | 1.00 | 22.67 |
| ATOM | 1689 | CA | VAL | 226 | 29.645 | −1.817 | 71.341 | 1.00 | 21.60 |
| ATOM | 1690 | CB | VAL | 226 | 30.618 | −2.993 | 71.634 | 1.00 | 21.77 |
| ATOM | 1691 | CG1 | VAL | 226 | 29.821 | −4.291 | 71.718 | 1.00 | 21.54 |
| ATOM | 1692 | CG2 | VAL | 226 | 31.663 | −3.113 | 70.541 | 1.00 | 17.23 |
| ATOM | 1693 | C | VAL | 226 | 28.838 | −1.493 | 72.604 | 1.00 | 21.49 |
| ATOM | 1694 | O | VAL | 226 | 29.316 | −1.633 | 73.723 | 1.00 | 18.90 |
| ATOM | 1695 | N | GLY | 227 | 27.615 | −1.016 | 72.402 | 1.00 | 25.39 |
| ATOM | 1696 | CA | GLY | 227 | 26.744 | −0.675 | 73.518 | 1.00 | 26.76 |
| ATOM | 1697 | C | GLY | 227 | 25.353 | −1.140 | 73.150 | 1.00 | 28.03 |
| ATOM | 1698 | O | GLY | 227 | 25.155 | −2.315 | 72.846 | 1.00 | 29.80 |
| ATOM | 1699 | N | THR | 228 | 24.384 | −0.235 | 73.161 | 1.00 | 27.62 |
| ATOM | 1700 | CA | THR | 228 | 23.031 | −0.607 | 72.788 | 1.00 | 27.59 |
| ATOM | 1701 | CB | THR | 228 | 22.083 | 0.601 | 72.911 | 1.00 | 29.15 |
| ATOM | 1702 | OG1 | THR | 228 | 21.937 | 0.932 | 74.294 | 1.00 | 32.52 |
| ATOM | 1703 | CG2 | THR | 228 | 20.719 | 0.291 | 72.339 | 1.00 | 28.08 |
| ATOM | 1704 | C | THR | 228 | 23.094 | −1.080 | 71.345 | 1.00 | 26.98 |
| ATOM | 1705 | O | THR | 228 | 22.460 | −2.065 | 70.960 | 1.00 | 27.95 |
| ATOM | 1706 | N | GLY | 229 | 23.890 | −0.374 | 70.554 | 1.00 | 26.02 |
| ATOM | 1707 | CA | GLY | 229 | 24.050 | −0.718 | 69.154 | 1.00 | 25.33 |
| ATOM | 1708 | C | GLY | 229 | 25.503 | −1.055 | 68.911 | 1.00 | 24.09 |
| ATOM | 1709 | O | GLY | 229 | 26.312 | −1.004 | 69.838 | 1.00 | 23.25 |
| ATOM | 1710 | N | CYS | 230 | 25.850 | −1.395 | 67.677 | 1.00 | 24.12 |
| ATOM | 1711 | CA | CYS | 230 | 27.235 | −1.750 | 67.376 | 1.00 | 23.83 |
| ATOM | 1712 | CB | CYS | 230 | 27.395 | −3.280 | 67.425 | 1.00 | 20.39 |
| ATOM | 1713 | SG | CYS | 230 | 29.076 | −3.879 | 67.182 | 1.00 | 25.34 |
| ATOM | 1714 | C | CYS | 230 | 27.627 | −1.204 | 66.010 | 1.00 | 20.45 |
| ATOM | 1715 | O | CYS | 230 | 26.919 | −1.406 | 65.035 | 1.00 | 20.28 |
| ATOM | 1716 | N | ASN | 231 | 28.763 | −0.526 | 65.935 | 1.00 | 23.86 |
| ATOM | 1717 | CA | ASN | 231 | 29.196 | 0.076 | 64.669 | 1.00 | 24.35 |
| ATOM | 1718 | CB | ASN | 231 | 28.267 | 1.261 | 64.355 | 1.00 | 25.51 |
| ATOM | 1719 | CG | ASN | 231 | 28.598 | 1.962 | 63.042 | 1.00 | 27.76 |
| ATOM | 1720 | OD1 | ASN | 231 | 28.930 | 1.331 | 62.039 | 1.00 | 24.60 |
| ATOM | 1721 | ND2 | ASN | 231 | 28.472 | 3.288 | 63.043 | 1.00 | 30.91 |
| ATOM | 1722 | C | ASN | 231 | 30.640 | 0.553 | 64.784 | 1.00 | 23.81 |
| ATOM | 1723 | O | ASN | 231 | 31.184 | 0.624 | 65.885 | 1.00 | 23.94 |
| ATOM | 1724 | N | ALA | 232 | 31.249 | 0.885 | 63.651 | 1.00 | 22.70 |
| ATOM | 1725 | CA | ALA | 232 | 32.626 | 1.359 | 63.636 | 1.00 | 25.15 |
| ATOM | 1726 | CB | ALA | 232 | 33.580 | 0.169 | 63.463 | 1.00 | 24.36 |
| ATOM | 1727 | C | ALA | 232 | 32.867 | 2.372 | 62.511 | 1.00 | 26.31 |
| ATOM | 1728 | O | ALA | 232 | 32.127 | 2.416 | 61.530 | 1.00 | 28.47 |
| ATOM | 1729 | N | CYS | 233 | 33.911 | 3.176 | 62.664 | 1.00 | 24.88 |
| ATOM | 1730 | CA | CYS | 233 | 34.291 | 4.160 | 61.653 | 1.00 | 26.51 |
| ATOM | 1731 | CB | CYS | 233 | 33.899 | 5.583 | 62.076 | 1.00 | 24.89 |
| ATOM | 1732 | SG | CYS | 233 | 34.875 | 6.224 | 63.436 | 1.00 | 25.76 |
| ATOM | 1733 | C | CYS | 233 | 35.805 | 4.055 | 61.555 | 1.00 | 25.08 |
| ATOM | 1734 | O | CYS | 233 | 36.450 | 3.564 | 62.480 | 1.00 | 25.19 |
| ATOM | 1735 | N | TYR | 234 | 36.373 | 4.505 | 60.442 | 1.00 | 25.32 |
| ATOM | 1736 | CA | TYR | 234 | 37.820 | 4.427 | 60.245 | 1.00 | 23.93 |
| ATOM | 1737 | CB | TYR | 234 | 38.200 | 3.020 | 59.760 | 1.00 | 20.70 |
| ATOM | 1738 | CG | TYR | 234 | 37.782 | 2.771 | 58.328 | 1.00 | 16.78 |
| ATOM | 1739 | CD1 | TYR | 234 | 38.712 | 2.786 | 57.302 | 1.00 | 18.75 |
| ATOM | 1740 | CE1 | TYR | 234 | 38.326 | 2.668 | 55.975 | 1.00 | 18.89 |
| ATOM | 1741 | CD2 | TYR | 234 | 36.443 | 2.622 | 57.990 | 1.00 | 19.60 |
| ATOM | 1742 | CE2 | TYR | 234 | 36.043 | 2.506 | 56.666 | 1.00 | 18.40 |
| ATOM | 1743 | CZ | TYR | 234 | 36.990 | 2.535 | 55.665 | 1.00 | 21.55 |
| ATOM | 1744 | OH | TYR | 234 | 36.603 | 2.479 | 54.346 | 1.00 | 23.25 |
| ATOM | 1745 | C | TYR | 234 | 38.254 | 5.452 | 59.194 | 1.00 | 26.41 |
| ATOM | 1746 | O | TYR | 234 | 37.436 | 5.929 | 58.404 | 1.00 | 27.14 |
| ATOM | 1747 | N | MET | 235 | 39.543 | 5.769 | 59.179 | 1.00 | 27.10 |
| ATOM | 1748 | CA | MET | 235 | 40.094 | 6.722 | 58.224 | 1.00 | 28.74 |
| ATOM | 1749 | CB | MET | 235 | 41.383 | 7.331 | 58.789 | 1.00 | 29.38 |
| ATOM | 1750 | CG | MET | 235 | 41.169 | 8.180 | 60.035 | 1.00 | 31.43 |
| ATOM | 1751 | SD | MET | 235 | 39.947 | 9.503 | 59.750 | 1.00 | 32.30 |
| ATOM | 1752 | CE | MET | 235 | 40.866 | 10.535 | 58.591 | 1.00 | 34.11 |
| ATOM | 1753 | C | MET | 235 | 40.374 | 6.066 | 56.869 | 1.00 | 29.42 |
| ATOM | 1754 | O | MET | 235 | 41.170 | 5.134 | 56.767 | 1.00 | 30.49 |
| ATOM | 1755 | N | GLU | 236 | 39.714 | 6.565 | 55.829 | 1.00 | 31.08 |
| ATOM | 1756 | CA | GLU | 236 | 39.867 | 6.040 | 54.476 | 1.00 | 31.04 |
| ATOM | 1757 | CB | GLU | 236 | 38.491 | 5.743 | 53.879 | 1.00 | 31.57 |
| ATOM | 1758 | CG | GLU | 236 | 38.536 | 5.161 | 52.474 | 1.00 | 32.18 |
| ATOM | 1759 | CD | GLU | 236 | 39.330 | 3.875 | 52.427 | 1.00 | 32.52 |
| ATOM | 1760 | OE1 | GLU | 236 | 40.565 | 3.952 | 52.273 | 1.00 | 34.34 |
| ATOM | 1761 | OE2 | GLU | 236 | 38.723 | 2.789 | 52.571 | 1.00 | 30.79 |
| ATOM | 1762 | C | GLU | 236 | 40.598 | 7.030 | 53.574 | 1.00 | 33.43 |
| ATOM | 1763 | O | GLU | 236 | 40.583 | 8.238 | 53.818 | 1.00 | 29.93 |
| ATOM | 1764 | N | GLU | 237 | 41.240 | 6.506 | 52.532 | 1.00 | 35.85 |
| ATOM | 1765 | CA | GLU | 237 | 41.969 | 7.333 | 51.575 | 1.00 | 37.83 |
| ATOM | 1766 | CB | GLU | 237 | 42.934 | 6.462 | 50.764 | 1.00 | 40.16 |
| ATOM | 1767 | CG | GLU | 237 | 43.684 | 5.426 | 51.602 | 1.00 | 43.86 |
| ATOM | 1768 | CD | GLU | 237 | 44.466 | 6.049 | 52.743 | 1.00 | 47.85 |
| ATOM | 1769 | OE1 | GLU | 237 | 44.806 | 5.322 | 53.704 | 1.00 | 51.02 |
| ATOM | 1770 | OE2 | GLU | 237 | 44.747 | 7.264 | 52.681 | 1.00 | 48.78 |
| ATOM | 1771 | C | GLU | 237 | 40.920 | 7.969 | 50.657 | 1.00 | 37.87 |
| ATOM | 1772 | O | GLU | 237 | 40.058 | 7.268 | 50.122 | 1.00 | 38.29 |
| ATOM | 1773 | N | MET | 238 | 40.987 | 9.287 | 50.477 | 1.00 | 37.42 |
| ATOM | 1774 | CA | MET | 238 | 40.009 | 9.987 | 49.644 | 1.00 | 37.50 |
| ATOM | 1775 | CB | MET | 238 | 40.375 | 11.467 | 49.501 | 1.00 | 38.62 |
| ATOM | 1776 | CG | MET | 238 | 39.772 | 12.355 | 50.587 | 1.00 | 40.32 |
| ATOM | 1777 | SD | MET | 238 | 37.956 | 12.144 | 50.764 | 1.00 | 42.83 |
| ATOM | 1778 | CE | MET | 238 | 37.308 | 13.116 | 49.410 | 1.00 | 44.06 |
| ATOM | 1779 | C | MET | 238 | 39.796 | 9.374 | 48.270 | 1.00 | 36.21 |
| ATOM | 1780 | O | MET | 238 | 38.685 | 9.413 | 47.740 | 1.00 | 33.93 |
| ATOM | 1781 | N | GLN | 239 | 40.848 | 8.803 | 47.690 | 1.00 | 35.50 |
| ATOM | 1782 | CA | GLN | 239 | 40.714 | 8.184 | 46.378 | 1.00 | 36.82 |
| ATOM | 1783 | CB | GLN | 239 | 42.078 | 7.732 | 45.846 | 1.00 | 39.35 |
| ATOM | 1784 | CG | GLN | 239 | 42.839 | 6.804 | 46.774 | 1.00 | 44.12 |
| ATOM | 1785 | CD | GLN | 239 | 43.900 | 7.534 | 47.584 | 1.00 | 49.18 |
| ATOM | 1786 | OE1 | GLN | 239 | 43.635 | 8.580 | 48.192 | 1.00 | 49.88 |
| ATOM | 1787 | NE2 | GLN | 239 | 45.111 | 6.981 | 47.600 | 1.00 | 49.95 |
| ATOM | 1788 | C | GLN | 239 | 39.762 | 6.986 | 46.395 | 1.00 | 35.72 |
| ATOM | 1789 | O | GLN | 239 | 39.276 | 6.568 | 45.348 | 1.00 | 37.20 |
| ATOM | 1790 | N | ASN | 240 | 39.503 | 6.419 | 47.570 | 1.00 | 34.56 |
| ATOM | 1791 | CA | ASN | 240 | 38.604 | 5.272 | 47.648 | 1.00 | 33.20 |
| ATOM | 1792 | CB | ASN | 240 | 39.118 | 4.239 | 48.658 | 1.00 | 33.68 |
| ATOM | 1793 | CG | ASN | 240 | 40.548 | 3.802 | 48.369 | 1.00 | 34.24 |
| ATOM | 1794 | OD1 | ASN | 240 | 40.963 | 3.710 | 47.210 | 1.00 | 33.87 |
| ATOM | 1795 | ND2 | ASN | 240 | 41.306 | 3.523 | 49.424 | 1.00 | 34.32 |
| ATOM | 1796 | C | ASN | 240 | 37.190 | 5.690 | 48.011 | 1.00 | 33.25 |
| ATOM | 1797 | O | ASN | 240 | 36.259 | 4.886 | 47.936 | 1.00 | 33.86 |
| ATOM | 1798 | N | VAL | 241 | 37.024 | 6.946 | 48.414 | 1.00 | 32.52 |
| ATOM | 1799 | CA | VAL | 241 | 35.702 | 7.441 | 48.753 | 1.00 | 31.62 |
| ATOM | 1800 | CB | VAL | 241 | 35.755 | 8.559 | 49.811 | 1.00 | 29.14 |
| ATOM | 1801 | CG1 | VAL | 241 | 34.339 | 8.948 | 50.204 | 1.00 | 31.00 |
| ATOM | 1802 | CG2 | VAL | 241 | 36.530 | 8.107 | 51.021 | 1.00 | 26.87 |
| ATOM | 1803 | C | VAL | 241 | 35.102 | 8.010 | 47.474 | 1.00 | 33.73 |
| ATOM | 1804 | O | VAL | 241 | 35.048 | 9.224 | 47.286 | 1.00 | 35.18 |
| ATOM | 1805 | N | GLU | 242 | 34.643 | 7.132 | 46.595 | 1.00 | 33.33 |
| ATOM | 1806 | CA | GLU | 242 | 34.075 | 7.572 | 45.324 | 1.00 | 33.69 |
| ATOM | 1807 | CB | GLU | 242 | 33.788 | 6.364 | 44.431 | 1.00 | 31.05 |
| ATOM | 1808 | CG | GLU | 242 | 34.983 | 5.457 | 44.222 | 1.00 | 33.00 |
| ATOM | 1809 | CD | GLU | 242 | 34.767 | 4.451 | 43.115 | 1.00 | 33.45 |
| ATOM | 1810 | OE1 | GLU | 242 | 33.595 | 4.162 | 42.776 | 1.00 | 33.74 |
| ATOM | 1811 | OE2 | GLU | 242 | 35.778 | 3.940 | 42.592 | 1.00 | 35.96 |
| ATOM | 1812 | C | GLU | 242 | 32.812 | 8.437 | 45.427 | 1.00 | 34.45 |
| ATOM | 1813 | O | GLU | 242 | 32.406 | 9.061 | 44.442 | 1.00 | 32.92 |
| ATOM | 1814 | N | LEU | 243 | 32.192 | 8.471 | 46.602 | 1.00 | 33.82 |
| ATOM | 1815 | CA | LEU | 243 | 30.982 | 9.262 | 46.799 | 1.00 | 36.13 |
| ATOM | 1816 | CB | LEU | 243 | 30.080 | 8.598 | 47.844 | 1.00 | 33.99 |
| ATOM | 1817 | CG | LEU | 243 | 29.168 | 7.490 | 47.297 | 1.00 | 37.04 |
| ATOM | 1818 | CD1 | LEU | 243 | 27.999 | 8.096 | 46.545 | 1.00 | 36.01 |
| ATOM | 1819 | CD2 | LEU | 243 | 29.969 | 6.560 | 46.384 | 1.00 | 36.49 |
| ATOM | 1820 | C | LEU | 243 | 31.290 | 10.700 | 47.199 | 1.00 | 35.69 |
| ATOM | 1821 | O | LEU | 243 | 30.406 | 11.458 | 47.585 | 1.00 | 37.51 |
| ATOM | 1822 | N | VAL | 244 | 32.560 | 11.062 | 47.117 | 1.00 | 37.53 |
| ATOM | 1823 | CA | VAL | 244 | 32.992 | 12.411 | 47.426 | 1.00 | 37.50 |
| ATOM | 1824 | CB | VAL | 244 | 33.537 | 12.547 | 48.861 | 1.00 | 36.75 |
| ATOM | 1825 | CG1 | VAL | 244 | 33.967 | 13.990 | 49.109 | 1.00 | 36.55 |
| ATOM | 1826 | CG2 | VAL | 244 | 32.465 | 12.160 | 49.870 | 1.00 | 37.02 |
| ATOM | 1827 | C | VAL | 244 | 34.099 | 12.727 | 46.446 | 1.00 | 39.75 |
| ATOM | 1828 | O | VAL | 244 | 35.090 | 12.003 | 46.361 | 1.00 | 39.55 |
| ATOM | 1829 | N | GLU | 245 | 33.909 | 13.802 | 45.688 | 1.00 | 42.16 |
| ATOM | 1830 | CA | GLU | 245 | 34.880 | 14.232 | 44.695 | 1.00 | 42.30 |
| ATOM | 1831 | CB | GLU | 245 | 34.372 | 15.487 | 43.989 | 1.00 | 45.34 |
| ATOM | 1832 | CG | GLU | 245 | 34.886 | 15.636 | 42.576 | 1.00 | 48.54 |
| ATOM | 1833 | CD | GLU | 245 | 34.377 | 16.893 | 41.901 | 1.00 | 50.12 |
| ATOM | 1834 | OE1 | GLU | 245 | 33.192 | 17.249 | 42.107 | 1.00 | 49.37 |
| ATOM | 1835 | OE2 | GLU | 245 | 35.164 | 17.511 | 41.152 | 1.00 | 52.40 |
| ATOM | 1836 | C | GLU | 245 | 36.203 | 14.532 | 45.378 | 1.00 | 41.00 |
| ATOM | 1837 | O | GLU | 245 | 36.230 | 15.132 | 46.446 | 1.00 | 42.20 |
| ATOM | 1838 | N | GLY | 246 | 37.297 | 14.107 | 44.761 | 1.00 | 41.28 |
| ATOM | 1839 | CA | GLY | 246 | 38.606 | 14.349 | 45.336 | 1.00 | 42.88 |
| ATOM | 1840 | C | GLY | 246 | 39.362 | 13.066 | 45.618 | 1.00 | 45.38 |
| ATOM | 1841 | O | GLY | 246 | 38.774 | 12.056 | 45.997 | 1.00 | 45.50 |
| ATOM | 1842 | N | ASP | 247 | 40.675 | 13.105 | 45.443 | 1.00 | 47.31 |
| ATOM | 1843 | CA | ASP | 247 | 41.509 | 11.940 | 45.687 | 1.00 | 49.13 |
| ATOM | 1844 | CB | ASP | 247 | 42.139 | 11.454 | 44.384 | 1.00 | 51.65 |
| ATOM | 1845 | CG | ASP | 247 | 41.131 | 10.836 | 43.449 | 1.00 | 56.09 |
| ATOM | 1846 | OD1 | ASP | 247 | 41.534 | 10.410 | 42.345 | 1.00 | 58.83 |
| ATOM | 1847 | OD2 | ASP | 247 | 39.936 | 10.770 | 43.819 | 1.00 | 59.44 |
| ATOM | 1848 | C | ASP | 247 | 42.611 | 12.274 | 46.667 | 1.00 | 49.51 |
| ATOM | 1849 | O | ASP | 247 | 43.406 | 11.415 | 47.039 | 1.00 | 49.57 |
| ATOM | 1850 | N | GLU | 248 | 42.661 | 13.531 | 47.086 | 1.00 | 50.49 |
| ATOM | 1851 | CA | GLU | 248 | 43.696 | 13.957 | 48.011 | 1.00 | 50.97 |
| ATOM | 1852 | CB | GLU | 248 | 44.198 | 15.351 | 47.634 | 1.00 | 54.71 |
| ATOM | 1853 | CG | GLU | 248 | 45.670 | 15.391 | 47.259 | 1.00 | 62.15 |
| ATOM | 1854 | CD | GLU | 248 | 46.067 | 14.259 | 46.317 | 1.00 | 66.63 |
| ATOM | 1855 | OE1 | GLU | 248 | 46.196 | 13.105 | 46.788 | 1.00 | 68.58 |
| ATOM | 1856 | OE2 | GLU | 248 | 46.241 | 14.520 | 45.105 | 1.00 | 68.21 |
| ATOM | 1857 | C | GLU | 248 | 43.222 | 13.955 | 49.446 | 1.00 | 47.90 |
| ATOM | 1858 | O | GLU | 248 | 42.063 | 14.250 | 49.726 | 1.00 | 46.55 |
| ATOM | 1859 | N | GLY | 249 | 44.133 | 13.614 | 50.351 | 1.00 | 45.49 |
| ATOM | 1860 | CA | GLY | 249 | 43.799 | 13.590 | 51.759 | 1.00 | 44.30 |
| ATOM | 1861 | C | GLY | 249 | 43.138 | 12.301 | 52.205 | 1.00 | 42.85 |
| ATOM | 1862 | O | GLY | 249 | 43.257 | 11.259 | 51.552 | 1.00 | 42.97 |
| ATOM | 1863 | N | ARG | 250 | 42.444 | 12.380 | 53.335 | 1.00 | 41.43 |
| ATOM | 1864 | CA | ARG | 250 | 41.747 | 11.232 | 53.897 | 1.00 | 39.63 |
| ATOM | 1865 | CB | ARG | 250 | 42.625 | 10.532 | 54.931 | 1.00 | 40.69 |
| ATOM | 1866 | CG | ARG | 250 | 44.092 | 10.454 | 54.559 | 1.00 | 43.91 |
| ATOM | 1867 | CD | ARG | 250 | 44.903 | 9.902 | 55.714 | 1.00 | 45.22 |
| ATOM | 1868 | NE | ARG | 250 | 44.630 | 8.487 | 55.940 | 1.00 | 45.43 |
| ATOM | 1869 | CZ | ARG | 250 | 45.040 | 7.813 | 57.007 | 1.00 | 44.67 |
| ATOM | 1870 | NH1 | ARG | 250 | 45.738 | 8.426 | 57.954 | 1.00 | 46.95 |
| ATOM | 1871 | NH2 | ARG | 250 | 44.761 | 6.524 | 57.121 | 1.00 | 46.99 |
| ATOM | 1872 | C | ARG | 250 | 40.486 | 11.726 | 54.580 | 1.00 | 37.70 |
| ATOM | 1873 | O | ARG | 250 | 40.430 | 12.865 | 55.042 | 1.00 | 37.51 |
| ATOM | 1874 | N | MET | 251 | 39.473 | 10.867 | 54.630 | 1.00 | 35.10 |
| ATOM | 1875 | CA | MET | 251 | 38.216 | 11.197 | 55.277 | 1.00 | 32.34 |
| ATOM | 1876 | CB | MET | 251 | 37.137 | 11.517 | 54.242 | 1.00 | 33.00 |
| ATOM | 1877 | CG | MET | 251 | 35.803 | 11.907 | 54.868 | 1.00 | 31.56 |
| ATOM | 1878 | SD | MET | 251 | 34.474 | 12.160 | 53.677 | 1.00 | 37.84 |
| ATOM | 1879 | CE | MET | 251 | 35.067 | 13.715 | 52.885 | 1.00 | 32.92 |
| ATOM | 1880 | C | MET | 251 | 37.764 | 10.007 | 56.121 | 1.00 | 32.47 |
| ATOM | 1881 | O | MET | 251 | 38.024 | 8.852 | 55.777 | 1.00 | 31.05 |
| ATOM | 1882 | N | CYS | 252 | 37.088 | 10.292 | 57.229 | 1.00 | 30.16 |
| ATOM | 1883 | CA | CYS | 252 | 36.595 | 9.236 | 58.092 | 1.00 | 30.32 |
| ATOM | 1884 | CB | CYS | 252 | 36.364 | 9.762 | 59.517 | 1.00 | 30.54 |
| ATOM | 1885 | SG | CYS | 252 | 35.601 | 8.557 | 60.676 | 1.00 | 28.61 |
| ATOM | 1886 | C | CYS | 252 | 35.292 | 8.717 | 57.511 | 1.00 | 29.86 |
| ATOM | 1887 | O | CYS | 252 | 34.422 | 9.495 | 57.114 | 1.00 | 29.84 |
| ATOM | 1888 | N | VAL | 253 | 35.170 | 7.397 | 57.438 | 1.00 | 28.79 |
| ATOM | 1889 | CA | VAL | 253 | 33.960 | 6.776 | 56.921 | 1.00 | 27.69 |
| ATOM | 1890 | CB | VAL | 253 | 34.291 | 5.761 | 55.816 | 1.00 | 28.07 |
| ATOM | 1891 | CG1 | VAL | 253 | 33.033 | 5.005 | 55.405 | 1.00 | 26.98 |
| ATOM | 1892 | CG2 | VAL | 253 | 34.898 | 6.484 | 54.624 | 1.00 | 24.14 |
| ATOM | 1893 | C | VAL | 253 | 33.200 | 6.069 | 58.038 | 1.00 | 28.79 |
| ATOM | 1894 | O | VAL | 253 | 33.801 | 5.448 | 58.922 | 1.00 | 31.23 |
| ATOM | 1895 | N | ASN | 254 | 31.879 | 6.188 | 58.000 | 1.00 | 28.38 |
| ATOM | 1896 | CA | ASN | 254 | 31.003 | 5.557 | 58.976 | 1.00 | 27.73 |
| ATOM | 1897 | CB | ASN | 254 | 29.834 | 6.473 | 59.328 | 1.00 | 27.41 |
| ATOM | 1898 | CG | ASN | 254 | 28.803 | 5.779 | 60.181 | 1.00 | 31.67 |
| ATOM | 1899 | OD1 | ASN | 254 | 29.048 | 4.675 | 60.677 | 1.00 | 32.14 |
| ATOM | 1900 | ND2 | ASN | 254 | 27.643 | 6.415 | 60.367 | 1.00 | 29.17 |
| ATOM | 1901 | C | ASN | 254 | 30.480 | 4.295 | 58.299 | 1.00 | 27.41 |
| ATOM | 1902 | O | ASN | 254 | 29.575 | 4.372 | 57.467 | 1.00 | 25.53 |
| ATOM | 1903 | N | THR | 255 | 31.049 | 3.142 | 58.654 | 1.00 | 24.66 |
| ATOM | 1904 | CA | THR | 255 | 30.662 | 1.883 | 58.016 | 1.00 | 24.86 |
| ATOM | 1905 | CB | THR | 255 | 31.501 | 0.665 | 58.527 | 1.00 | 23.42 |
| ATOM | 1906 | OG1 | THR | 255 | 31.071 | 0.310 | 59.849 | 1.00 | 23.50 |
| ATOM | 1907 | CG2 | THR | 255 | 32.973 | 0.982 | 58.558 | 1.00 | 23.88 |
| ATOM | 1908 | C | THR | 255 | 29.207 | 1.488 | 58.195 | 1.00 | 23.00 |
| ATOM | 1909 | O | THR | 255 | 28.589 | 0.984 | 57.259 | 1.00 | 24.38 |
| ATOM | 1910 | N | GLU | 256 | 28.673 | 1.710 | 59.394 | 1.00 | 23.70 |
| ATOM | 1911 | CA | GLU | 256 | 27.306 | 1.305 | 59.721 | 1.00 | 26.37 |
| ATOM | 1912 | CB | GLU | 256 | 26.271 | 2.017 | 58.838 | 1.00 | 26.22 |
| ATOM | 1913 | CG | GLU | 256 | 25.974 | 3.471 | 59.204 | 1.00 | 29.32 |
| ATOM | 1914 | CD | GLU | 256 | 25.284 | 3.644 | 60.558 | 1.00 | 31.10 |
| ATOM | 1915 | OE1 | GLU | 256 | 24.489 | 2.764 | 60.953 | 1.00 | 31.47 |
| ATOM | 1916 | OE2 | GLU | 256 | 25.523 | 4.682 | 61.218 | 1.00 | 30.04 |
| ATOM | 1917 | C | GLU | 256 | 27.269 | −0.203 | 59.458 | 1.00 | 27.40 |
| ATOM | 1918 | O | GLU | 256 | 26.369 | −0.713 | 58.782 | 1.00 | 26.71 |
| ATOM | 1919 | N | TRP | 257 | 28.269 | −0.912 | 59.982 | 1.00 | 25.98 |
| ATOM | 1920 | CA | TRP | 257 | 28.335 | −2.356 | 59.774 | 1.00 | 24.56 |
| ATOM | 1921 | CB | TRP | 257 | 29.714 | −2.928 | 60.180 | 1.00 | 21.05 |
| ATOM | 1922 | CG | TRP | 257 | 30.100 | −2.891 | 61.653 | 1.00 | 17.51 |
| ATOM | 1923 | CD2 | TRP | 257 | 31.429 | −3.026 | 62.182 | 1.00 | 16.19 |
| ATOM | 1924 | CE2 | TRP | 257 | 31.320 | −3.077 | 63.588 | 1.00 | 14.42 |
| ATOM | 1925 | CE3 | TRP | 257 | 32.705 | −3.112 | 61.597 | 1.00 | 16.23 |
| ATOM | 1926 | CD1 | TRP | 257 | 29.264 | −2.862 | 62.733 | 1.00 | 18.14 |
| ATOM | 1927 | NE1 | TRP | 257 | 29.990 | −2.977 | 63.902 | 1.00 | 19.95 |
| ATOM | 1928 | CZ2 | TRP | 257 | 32.435 | −3.214 | 64.421 | 1.00 | 17.46 |
| ATOM | 1929 | CZ3 | TRP | 257 | 33.815 | −3.246 | 62.424 | 1.00 | 13.91 |
| ATOM | 1930 | CH2 | TRP | 257 | 33.672 | −3.294 | 63.822 | 1.00 | 14.28 |
| ATOM | 1931 | C | TRP | 257 | 27.218 | −3.091 | 60.500 | 1.00 | 24.58 |
| ATOM | 1932 | O | TRP | 257 | 27.067 | −4.305 | 60.352 | 1.00 | 24.81 |
| ATOM | 1933 | N | GLY | 258 | 26.427 | −2.354 | 61.273 | 1.00 | 23.21 |
| ATOM | 1934 | CA | GLY | 258 | 25.328 | −2.981 | 61.982 | 1.00 | 23.11 |
| ATOM | 1935 | C | GLY | 258 | 24.385 | −3.640 | 60.991 | 1.00 | 25.72 |
| ATOM | 1936 | O | GLY | 258 | 23.758 | −4.660 | 61.285 | 1.00 | 28.37 |
| ATOM | 1937 | N | ALA | 259 | 24.288 | −3.067 | 59.796 | 1.00 | 24.64 |
| ATOM | 1938 | CA | ALA | 259 | 23.406 | −3.630 | 58.789 | 1.00 | 25.53 |
| ATOM | 1939 | CB | ALA | 259 | 22.866 | −2.519 | 57.874 | 1.00 | 25.11 |
| ATOM | 1940 | C | ALA | 259 | 24.084 | −4.724 | 57.961 | 1.00 | 25.44 |
| ATOM | 1941 | O | ALA | 259 | 23.515 | −5.205 | 56.985 | 1.00 | 24.68 |
| ATOM | 1942 | N | PHE | 260 | 25.306 | −5.101 | 58.329 | 1.00 | 26.96 |
| ATOM | 1943 | CA | PHE | 260 | 25.995 | −6.175 | 57.614 | 1.00 | 28.11 |
| ATOM | 1944 | CB | PHE | 260 | 27.359 | −6.440 | 58.254 | 1.00 | 30.88 |
| ATOM | 1945 | CG | PHE | 260 | 28.127 | −7.569 | 57.625 | 1.00 | 33.87 |
| ATOM | 1946 | CD1 | PHE | 260 | 28.496 | −7.525 | 56.286 | 1.00 | 33.60 |
| ATOM | 1947 | CD2 | PHE | 260 | 28.499 | −8.675 | 58.380 | 1.00 | 37.30 |
| ATOM | 1948 | CE1 | PHE | 260 | 29.220 | −8.564 | 55.716 | 1.00 | 33.58 |
| ATOM | 1949 | CE2 | PHE | 260 | 29.229 | −9.720 | 57.808 | 1.00 | 35.65 |
| ATOM | 1950 | CZ | PHE | 260 | 29.586 | −9.660 | 56.478 | 1.00 | 34.41 |
| ATOM | 1951 | C | PHE | 260 | 25.080 | −7.388 | 57.783 | 1.00 | 28.87 |
| ATOM | 1952 | O | PHE | 260 | 24.487 | −7.576 | 58.849 | 1.00 | 27.08 |
| ATOM | 1953 | N | GLY | 261 | 24.941 | −8.193 | 56.737 | 1.00 | 28.88 |
| ATOM | 1954 | CA | GLY | 261 | 24.074 | −9.357 | 56.826 | 1.00 | 30.83 |
| ATOM | 1955 | C | GLY | 261 | 22.664 | −9.092 | 56.317 | 1.00 | 32.15 |
| ATOM | 1956 | O | GLY | 261 | 21.905 | −10.021 | 56.043 | 1.00 | 34.22 |
| ATOM | 1957 | N | ASP | 262 | 22.307 | −7.822 | 56.175 | 1.00 | 33.45 |
| ATOM | 1958 | CA | ASP | 262 | 20.975 | −7.456 | 55.701 | 1.00 | 35.91 |
| ATOM | 1959 | CB | ASP | 262 | 20.761 | −5.948 | 55.868 | 1.00 | 35.78 |
| ATOM | 1960 | CG | ASP | 262 | 20.674 | −5.541 | 57.323 | 1.00 | 35.93 |
| ATOM | 1961 | OD1 | ASP | 262 | 20.903 | −6.415 | 58.182 | 1.00 | 37.70 |
| ATOM | 1962 | OD2 | ASP | 262 | 20.382 | −4.364 | 57.615 | 1.00 | 35.14 |
| ATOM | 1963 | C | ASP | 262 | 20.676 | −7.884 | 54.262 | 1.00 | 36.35 |
| ATOM | 1964 | O | ASP | 262 | 19.546 | −7.758 | 53.799 | 1.00 | 37.40 |
| ATOM | 1965 | N | SER | 263 | 21.685 | −8.380 | 53.554 | 1.00 | 37.07 |
| ATOM | 1966 | CA | SER | 263 | 21.488 | −8.863 | 52.189 | 1.00 | 37.53 |
| ATOM | 1967 | CB | SER | 263 | 22.420 | −8.155 | 51.200 | 1.00 | 37.00 |
| ATOM | 1968 | OG | SER | 263 | 22.028 | −6.815 | 50.991 | 1.00 | 38.85 |
| ATOM | 1969 | C | SER | 263 | 21.770 | −10.359 | 52.161 | 1.00 | 37.06 |
| ATOM | 1970 | O | SER | 263 | 22.062 | −10.923 | 51.107 | 1.00 | 36.90 |
| ATOM | 1971 | N | GLY | 264 | 21.697 | −10.988 | 53.331 | 1.00 | 36.97 |
| ATOM | 1972 | CA | GLY | 264 | 21.934 | −12.418 | 53.428 | 1.00 | 37.50 |
| ATOM | 1973 | C | GLY | 264 | 23.370 | −12.857 | 53.663 | 1.00 | 38.59 |
| ATOM | 1974 | O | GLY | 264 | 23.666 | −14.050 | 53.573 | 1.00 | 40.28 |
| ATOM | 1975 | N | GLU | 265 | 24.263 | −11.915 | 53.961 | 1.00 | 37.52 |
| ATOM | 1976 | CA | GLU | 265 | 25.671 | −12.237 | 54.199 | 1.00 | 36.34 |
| ATOM | 1977 | CB | GLU | 265 | 26.488 | −10.965 | 54.438 | 1.00 | 35.82 |
| ATOM | 1978 | CG | GLU | 265 | 26.535 | −9.976 | 53.289 | 1.00 | 38.57 |
| ATOM | 1979 | CD | GLU | 265 | 25.270 | −9.148 | 53.158 | 1.00 | 39.55 |
| ATOM | 1980 | OE1 | GLU | 265 | 24.600 | −8.901 | 54.173 | 1.00 | 38.51 |
| ATOM | 1981 | OE2 | GLU | 265 | 24.953 | −8.722 | 52.031 | 1.00 | 43.82 |
| ATOM | 1982 | C | GLU | 265 | 25.906 | −13.171 | 55.391 | 1.00 | 36.38 |
| ATOM | 1983 | O | GLU | 265 | 26.899 | −13.906 | 55.425 | 1.00 | 35.35 |
| ATOM | 1984 | N | LEU | 266 | 24.996 | −13.140 | 56.362 | 1.00 | 34.63 |
| ATOM | 1985 | CA | LEU | 266 | 25.130 | −13.955 | 57.567 | 1.00 | 35.02 |
| ATOM | 1986 | CB | LEU | 266 | 25.008 | −13.054 | 58.803 | 1.00 | 31.68 |
| ATOM | 1987 | CG | LEU | 266 | 26.017 | −11.914 | 58.973 | 1.00 | 33.35 |
| ATOM | 1988 | CD1 | LEU | 266 | 25.555 | −10.975 | 60.077 | 1.00 | 32.52 |
| ATOM | 1989 | CD2 | LEU | 266 | 27.383 | −12.480 | 59.294 | 1.00 | 32.43 |
| ATOM | 1990 | C | LEU | 266 | 24.108 | −15.092 | 57.674 | 1.00 | 35.37 |
| ATOM | 1991 | O | LEU | 266 | 24.047 | −15.779 | 58.696 | 1.00 | 35.21 |
| ATOM | 1992 | N | ASP | 267 | 23.321 | −15.300 | 56.627 | 1.00 | 36.35 |
| ATOM | 1993 | CA | ASP | 267 | 22.286 | −16.332 | 56.643 | 1.00 | 39.50 |
| ATOM | 1994 | CB | ASP | 267 | 21.664 | −16.480 | 55.248 | 1.00 | 42.21 |
| ATOM | 1995 | CG | ASP | 267 | 20.666 | −15.369 | 54.921 | 1.00 | 45.43 |
| ATOM | 1996 | OD1 | ASP | 267 | 20.205 | −15.320 | 53.759 | 1.00 | 48.41 |
| ATOM | 1997 | OD2 | ASP | 267 | 20.332 | −14.554 | 55.813 | 1.00 | 45.57 |
| ATOM | 1998 | C | ASP | 267 | 22.676 | −17.715 | 57.171 | 1.00 | 38.87 |
| ATOM | 1999 | O | ASP | 267 | 21.888 | −18.353 | 57.867 | 1.00 | 39.64 |
| ATOM | 2000 | N | GLU | 268 | 23.879 | −18.179 | 56.860 | 1.00 | 38.72 |
| ATOM | 2001 | CA | GLU | 268 | 24.301 | −19.502 | 57.313 | 1.00 | 39.91 |
| ATOM | 2002 | CB | GLU | 268 | 25.510 | −19.971 | 56.495 | 1.00 | 40.60 |
| ATOM | 2003 | CG | GLU | 268 | 26.847 | −19.444 | 56.976 | 1.00 | 43.85 |
| ATOM | 2004 | CD | GLU | 268 | 27.969 | −19.710 | 55.981 | 1.00 | 47.64 |
| ATOM | 2005 | OE1 | GLU | 268 | 28.013 | −19.017 | 54.941 | 1.00 | 49.73 |
| ATOM | 2006 | OE2 | GLU | 268 | 28.802 | −20.612 | 56.232 | 1.00 | 48.40 |
| ATOM | 2007 | C | GLU | 268 | 24.633 | −19.577 | 58.807 | 1.00 | 40.59 |
| ATOM | 2008 | O | GLU | 268 | 24.790 | −20.667 | 59.360 | 1.00 | 41.43 |
| ATOM | 2009 | N | PHE | 269 | 24.734 | −18.427 | 59.462 | 1.00 | 39.17 |
| ATOM | 2010 | CA | PHE | 269 | 25.070 | −18.402 | 60.882 | 1.00 | 37.75 |
| ATOM | 2011 | CB | PHE | 269 | 26.182 | −17.385 | 61.127 | 1.00 | 34.69 |
| ATOM | 2012 | CG | PHE | 269 | 27.435 | −17.675 | 60.369 | 1.00 | 35.74 |
| ATOM | 2013 | CD1 | PHE | 269 | 28.144 | −18.853 | 60.599 | 1.00 | 35.94 |
| ATOM | 2014 | CD2 | PHE | 269 | 27.910 | −16.781 | 59.416 | 1.00 | 34.75 |
| ATOM | 2015 | CE1 | PHE | 269 | 29.306 | −19.136 | 59.891 | 1.00 | 34.71 |
| ATOM | 2016 | CE2 | PHE | 269 | 29.068 | −17.050 | 58.701 | 1.00 | 34.58 |
| ATOM | 2017 | CZ | PHE | 269 | 29.770 | −18.233 | 58.939 | 1.00 | 35.80 |
| ATOM | 2018 | C | PHE | 269 | 23.898 | −18.085 | 61.793 | 1.00 | 36.73 |
| ATOM | 2019 | O | PHE | 269 | 23.932 | −18.384 | 62.984 | 1.00 | 36.59 |
| ATOM | 2020 | N | LEU | 270 | 22.861 | −17.480 | 61.231 | 1.00 | 37.18 |
| ATOM | 2021 | CA | LEU | 270 | 21.696 | −17.107 | 62.012 | 1.00 | 37.71 |
| ATOM | 2022 | CB | LEU | 270 | 20.712 | −16.332 | 61.135 | 1.00 | 36.52 |
| ATOM | 2023 | CG | LEU | 270 | 21.264 | −15.036 | 60.521 | 1.00 | 37.18 |
| ATOM | 2024 | CD1 | LEU | 270 | 20.299 | −14.516 | 59.466 | 1.00 | 38.72 |
| ATOM | 2025 | CD2 | LEU | 270 | 21.488 | −13.990 | 61.604 | 1.00 | 34.72 |
| ATOM | 2026 | C | LEU | 270 | 21.010 | −18.312 | 62.644 | 1.00 | 38.27 |
| ATOM | 2027 | O | LEU | 270 | 20.794 | −19.333 | 61.995 | 1.00 | 39.49 |
| ATOM | 2028 | N | LEU | 271 | 20.685 | −18.176 | 63.924 | 1.00 | 37.92 |
| ATOM | 2029 | CA | LEU | 271 | 20.010 | −19.212 | 64.693 | 1.00 | 38.22 |
| ATOM | 2030 | CB | LEU | 271 | 20.657 | −19.339 | 66.078 | 1.00 | 37.71 |
| ATOM | 2031 | CG | LEU | 271 | 21.897 | −20.220 | 66.261 | 1.00 | 38.14 |
| ATOM | 2032 | CD1 | LEU | 271 | 22.827 | −20.111 | 65.075 | 1.00 | 39.09 |
| ATOM | 2033 | CD2 | LEU | 271 | 22.596 | −19.830 | 67.549 | 1.00 | 35.73 |
| ATOM | 2034 | C | LEU | 271 | 18.536 | −18.845 | 64.855 | 1.00 | 39.78 |
| ATOM | 2035 | O | LEU | 271 | 18.125 | −17.721 | 64.538 | 1.00 | 38.05 |
| ATOM | 2036 | N | GLU | 272 | 17.751 | −19.794 | 65.358 | 1.00 | 39.69 |
| ATOM | 2037 | CA | GLU | 272 | 16.322 | −19.590 | 65.575 | 1.00 | 41.03 |
| ATOM | 2038 | CB | GLU | 272 | 15.697 | −20.842 | 66.219 | 1.00 | 43.64 |
| ATOM | 2039 | CG | GLU | 272 | 16.221 | −21.179 | 67.627 | 1.00 | 47.44 |
| ATOM | 2040 | CD | GLU | 272 | 15.685 | −22.509 | 68.182 | 1.00 | 49.81 |
| ATOM | 2041 | OE1 | GLU | 272 | 16.081 | −23.580 | 67.666 | 1.00 | 51.29 |
| ATOM | 2042 | OE2 | GLU | 272 | 14.869 | −22.484 | 69.134 | 1.00 | 47.60 |
| ATOM | 2043 | C | GLU | 272 | 16.084 | −18.377 | 66.466 | 1.00 | 39.89 |
| ATOM | 2044 | O | GLU | 272 | 15.151 | −17.602 | 66.250 | 1.00 | 40.35 |
| ATOM | 2045 | N | TYR | 273 | 16.944 | −18.208 | 67.465 | 1.00 | 38.65 |
| ATOM | 2046 | CA | TYR | 273 | 16.813 | −17.095 | 68.393 | 1.00 | 35.97 |
| ATOM | 2047 | CB | TYR | 273 | 17.829 | −17.238 | 69.530 | 1.00 | 35.50 |
| ATOM | 2048 | CG | TYR | 273 | 18.008 | −18.658 | 70.009 | 1.00 | 34.45 |
| ATOM | 2049 | CD1 | TYR | 273 | 19.109 | −19.416 | 69.611 | 1.00 | 32.53 |
| ATOM | 2050 | CE1 | TYR | 273 | 19.252 | −20.740 | 70.017 | 1.00 | 35.58 |
| ATOM | 2051 | CD2 | TYR | 273 | 17.053 | −19.258 | 70.830 | 1.00 | 34.35 |
| ATOM | 2052 | CE2 | TYR | 273 | 17.185 | −20.580 | 71.241 | 1.00 | 34.82 |
| ATOM | 2053 | CZ | TYR | 273 | 18.281 | −21.314 | 70.830 | 1.00 | 35.96 |
| ATOM | 2054 | OH | TYR | 273 | 18.381 | −22.626 | 71.208 | 1.00 | 38.31 |
| ATOM | 2055 | C | TYR | 273 | 17.021 | −15.763 | 67.680 | 1.00 | 35.11 |
| ATOM | 2056 | O | TYR | 273 | 16.404 | −14.752 | 68.031 | 1.00 | 34.85 |
| ATOM | 2057 | N | ASP | 274 | 17.888 | −15.763 | 66.676 | 1.00 | 36.47 |
| ATOM | 2058 | CA | ASP | 274 | 18.164 | −14.541 | 65.933 | 1.00 | 36.65 |
| ATOM | 2059 | CB | ASP | 274 | 19.405 | −14.718 | 65.059 | 1.00 | 32.36 |
| ATOM | 2060 | CG | ASP | 274 | 20.627 | −15.072 | 65.869 | 1.00 | 32.89 |
| ATOM | 2061 | OD1 | ASP | 274 | 20.949 | −14.315 | 66.810 | 1.00 | 30.53 |
| ATOM | 2062 | OD2 | ASP | 274 | 21.265 | −16.104 | 65.569 | 1.00 | 32.08 |
| ATOM | 2063 | C | ASP | 274 | 16.968 | −14.165 | 65.081 | 1.00 | 37.27 |
| ATOM | 2064 | O | ASP | 274 | 16.571 | −13.001 | 65.040 | 1.00 | 37.20 |
| ATOM | 2065 | N | ARG | 275 | 16.380 | −15.148 | 64.410 | 1.00 | 39.32 |
| ATOM | 2066 | CA | ARG | 275 | 15.222 | −14.866 | 63.574 | 1.00 | 41.70 |
| ATOM | 2067 | CB | ARG | 275 | 14.803 | −16.121 | 62.809 | 1.00 | 44.47 |
| ATOM | 2068 | CG | ARG | 275 | 15.908 | −16.666 | 61.914 | 1.00 | 49.05 |
| ATOM | 2069 | CD | ARG | 275 | 15.516 | −18.002 | 61.303 | 1.00 | 53.46 |
| ATOM | 2070 | NE | ARG | 275 | 16.668 | −18.740 | 60.779 | 1.00 | 57.36 |
| ATOM | 2071 | CZ | ARG | 275 | 17.352 | −18.408 | 59.685 | 1.00 | 58.81 |
| ATOM | 2072 | NH1 | ARG | 275 | 18.383 | −19.148 | 59.296 | 1.00 | 60.43 |
| ATOM | 2073 | NH2 | ARG | 275 | 17.005 | −17.341 | 58.976 | 1.00 | 61.75 |
| ATOM | 2074 | C | ARG | 275 | 14.079 | −14.353 | 64.446 | 1.00 | 41.43 |
| ATOM | 2075 | O | ARG | 275 | 13.350 | −13.444 | 64.059 | 1.00 | 40.04 |
| ATOM | 2076 | N | LEU | 276 | 13.939 | −14.927 | 65.637 | 1.00 | 40.97 |
| ATOM | 2077 | CA | LEU | 276 | 12.888 | −14.507 | 66.556 | 1.00 | 42.14 |
| ATOM | 2078 | CB | LEU | 276 | 12.831 | −15.450 | 67.761 | 1.00 | 44.12 |
| ATOM | 2079 | CG | LEU | 276 | 12.315 | −16.862 | 67.468 | 1.00 | 47.86 |
| ATOM | 2080 | CD1 | LEU | 276 | 12.662 | −17.800 | 68.618 | 1.00 | 48.62 |
| ATOM | 2081 | CD2 | LEU | 276 | 10.808 | −16.808 | 67.236 | 1.00 | 47.43 |
| ATOM | 2082 | C | LEU | 276 | 13.094 | −13.072 | 67.034 | 1.00 | 40.87 |
| ATOM | 2083 | O | LEU | 276 | 12.152 | −12.281 | 67.072 | 1.00 | 41.20 |
| ATOM | 2084 | N | VAL | 277 | 14.322 | −12.740 | 67.412 | 1.00 | 39.68 |
| ATOM | 2085 | CA | VAL | 277 | 14.617 | −11.390 | 67.876 | 1.00 | 40.86 |
| ATOM | 2086 | CB | VAL | 277 | 16.084 | −11.263 | 68.331 | 1.00 | 41.86 |
| ATOM | 2087 | CG1 | VAL | 277 | 16.447 | −9.802 | 68.497 | 1.00 | 43.25 |
| ATOM | 2088 | CG2 | VAL | 277 | 16.290 | −12.012 | 69.647 | 1.00 | 41.47 |
| ATOM | 2089 | C | VAL | 277 | 14.363 | −10.381 | 66.761 | 1.00 | 40.10 |
| ATOM | 2090 | O | VAL | 277 | 13.813 | −9.305 | 66.993 | 1.00 | 41.12 |
| ATOM | 2091 | N | ASP | 278 | 14.767 | −10.738 | 65.550 | 1.00 | 39.42 |
| ATOM | 2092 | CA | ASP | 278 | 14.592 | −9.867 | 64.398 | 1.00 | 40.24 |
| ATOM | 2093 | CB | ASP | 278 | 15.356 | −10.434 | 63.195 | 1.00 | 38.24 |
| ATOM | 2094 | CG | ASP | 278 | 15.179 | −9.598 | 61.943 | 1.00 | 40.23 |
| ATOM | 2095 | OD1 | ASP | 278 | 15.260 | −8.351 | 62.043 | 1.00 | 39.72 |
| ATOM | 2096 | OD2 | ASP | 278 | 14.969 | −10.187 | 60.860 | 1.00 | 38.10 |
| ATOM | 2097 | C | ASP | 278 | 13.120 | −9.669 | 64.043 | 1.00 | 41.19 |
| ATOM | 2098 | O | ASP | 278 | 12.693 | −8.545 | 63.791 | 1.00 | 40.82 |
| ATOM | 2099 | N | GLU | 279 | 12.347 | −10.754 | 64.035 | 1.00 | 43.34 |
| ATOM | 2100 | CA | GLU | 279 | 10.922 | −10.688 | 63.696 | 1.00 | 46.81 |
| ATOM | 2101 | CB | GLU | 279 | 10.321 | −12.097 | 63.627 | 1.00 | 50.53 |
| ATOM | 2102 | CG | GLU | 279 | 10.870 | −12.965 | 62.496 | 1.00 | 56.10 |
| ATOM | 2103 | CD | GLU | 279 | 10.320 | −14.382 | 62.523 | 1.00 | 59.07 |
| ATOM | 2104 | OE1 | GLU | 279 | 10.336 | −15.006 | 63.607 | 1.00 | 60.28 |
| ATOM | 2105 | OE2 | GLU | 279 | 9.880 | −14.876 | 61.461 | 1.00 | 60.79 |
| ATOM | 2106 | C | GLU | 279 | 10.086 | −9.840 | 64.652 | 1.00 | 47.25 |
| ATOM | 2107 | O | GLU | 279 | 9.048 | −9.303 | 64.260 | 1.00 | 46.34 |
| ATOM | 2108 | N | SER | 280 | 10.535 | −9.722 | 65.899 | 1.00 | 46.87 |
| ATOM | 2109 | CA | SER | 280 | 9.809 | −8.948 | 66.900 | 1.00 | 47.53 |
| ATOM | 2110 | CB | SER | 280 | 9.769 | −9.708 | 68.228 | 1.00 | 49.98 |
| ATOM | 2111 | OG | SER | 280 | 9.043 | −10.919 | 68.093 | 1.00 | 52.36 |
| ATOM | 2112 | C | SER | 280 | 10.415 | −7.575 | 67.129 | 1.00 | 47.33 |
| ATOM | 2113 | O | SER | 280 | 9.909 | −6.788 | 67.936 | 1.00 | 45.86 |
| ATOM | 2114 | N | SER | 281 | 11.499 | −7.289 | 66.416 | 1.00 | 46.95 |
| ATOM | 2115 | CA | SER | 281 | 12.172 | −6.004 | 66.552 | 1.00 | 46.75 |
| ATOM | 2116 | CB | SER | 281 | 13.581 | −6.081 | 65.971 | 1.00 | 47.24 |
| ATOM | 2117 | OG | SER | 281 | 13.524 | −6.172 | 64.559 | 1.00 | 47.80 |
| ATOM | 2118 | C | SER | 281 | 11.391 | −4.915 | 65.824 | 1.00 | 45.65 |
| ATOM | 2119 | O | SER | 281 | 10.514 | −5.199 | 65.013 | 1.00 | 45.10 |
| ATOM | 2120 | N | ALA | 282 | 11.723 | −3.667 | 66.123 | 1.00 | 45.75 |
| ATOM | 2121 | CA | ALA | 282 | 11.066 | −2.530 | 65.500 | 1.00 | 45.70 |
| ATOM | 2122 | CB | ALA | 282 | 11.257 | −1.289 | 66.354 | 1.00 | 45.60 |
| ATOM | 2123 | C | ALA | 282 | 11.617 | −2.286 | 64.100 | 1.00 | 46.48 |
| ATOM | 2124 | O | ALA | 282 | 11.252 | −1.303 | 63.449 | 1.00 | 48.61 |
| ATOM | 2125 | N | ASN | 283 | 12.493 | −3.172 | 63.633 | 1.00 | 43.90 |
| ATOM | 2126 | CA | ASN | 283 | 13.076 | −3.015 | 62.306 | 1.00 | 41.45 |
| ATOM | 2127 | CB | ASN | 283 | 14.300 | −2.092 | 62.384 | 1.00 | 40.08 |
| ATOM | 2128 | CG | ASN | 283 | 15.398 | −2.631 | 63.289 | 1.00 | 39.25 |
| ATOM | 2129 | OD1 | ASN | 283 | 15.136 | −3.308 | 64.289 | 1.00 | 37.65 |
| ATOM | 2130 | ND2 | ASN | 283 | 16.641 | −2.310 | 62.950 | 1.00 | 37.96 |
| ATOM | 2131 | C | ASN | 283 | 13.433 | −4.350 | 61.655 | 1.00 | 41.06 |
| ATOM | 2132 | O | ASN | 283 | 14.585 | −4.606 | 61.318 | 1.00 | 40.48 |
| ATOM | 2133 | N | PRO | 284 | 12.423 | −5.211 | 61.455 | 1.00 | 40.23 |
| ATOM | 2134 | CD | PRO | 284 | 11.013 | −4.898 | 61.751 | 1.00 | 40.75 |
| ATOM | 2135 | CA | PRO | 284 | 12.534 | −6.540 | 60.851 | 1.00 | 40.08 |
| ATOM | 2136 | CB | PRO | 284 | 11.080 | −6.914 | 60.581 | 1.00 | 40.52 |
| ATOM | 2137 | CG | PRO | 284 | 10.364 | −6.260 | 61.712 | 1.00 | 41.21 |
| ATOM | 2138 | C | PRO | 284 | 13.366 | −6.565 | 59.579 | 1.00 | 39.55 |
| ATOM | 2139 | O | PRO | 284 | 13.054 | −5.868 | 58.617 | 1.00 | 40.95 |
| ATOM | 2140 | N | GLY | 285 | 14.416 | −7.382 | 59.576 | 1.00 | 38.56 |
| ATOM | 2141 | CA | GLY | 285 | 15.266 | −7.491 | 58.407 | 1.00 | 35.73 |
| ATOM | 2142 | C | GLY | 285 | 16.428 | −6.516 | 58.371 | 1.00 | 35.10 |
| ATOM | 2143 | O | GLY | 285 | 17.288 | −6.624 | 57.500 | 1.00 | 36.22 |
| ATOM | 2144 | N | GLN | 286 | 16.468 | −5.573 | 59.308 | 1.00 | 34.06 |
| ATOM | 2145 | CA | GLN | 286 | 17.547 | −4.584 | 59.348 | 1.00 | 34.96 |
| ATOM | 2146 | CB | GLN | 286 | 16.974 | −3.166 | 59.321 | 1.00 | 39.16 |
| ATOM | 2147 | CG | GLN | 286 | 16.189 | −2.825 | 58.067 | 1.00 | 45.72 |
| ATOM | 2148 | CD | GLN | 286 | 15.698 | −1.384 | 58.074 | 1.00 | 51.15 |
| ATOM | 2149 | OE1 | GLN | 286 | 14.816 | −1.018 | 58.860 | 1.00 | 52.21 |
| ATOM | 2150 | NE2 | GLN | 286 | 16.276 | −0.555 | 57.203 | 1.00 | 50.85 |
| ATOM | 2151 | C | GLN | 286 | 18.439 | −4.719 | 60.573 | 1.00 | 33.59 |
| ATOM | 2152 | O | GLN | 286 | 17.993 | −5.157 | 61.637 | 1.00 | 33.18 |
| ATOM | 2153 | N | GLN | 287 | 19.701 | −4.334 | 60.408 | 1.00 | 32.85 |
| ATOM | 2154 | CA | GLN | 287 | 20.691 | −4.375 | 61.484 | 1.00 | 32.45 |
| ATOM | 2155 | CB | GLN | 287 | 20.248 | −3.456 | 62.636 | 1.00 | 33.34 |
| ATOM | 2156 | CG | GLN | 287 | 19.955 | −1.999 | 62.251 | 1.00 | 31.48 |
| ATOM | 2157 | CD | GLN | 287 | 21.188 | −1.259 | 61.743 | 1.00 | 31.78 |
| ATOM | 2158 | OE1 | GLN | 287 | 21.330 | −1.010 | 60.544 | 1.00 | 33.25 |
| ATOM | 2159 | NE2 | GLN | 287 | 22.090 | −0.921 | 62.652 | 1.00 | 27.51 |
| ATOM | 2160 | C | GLN | 287 | 20.924 | −5.788 | 62.032 | 1.00 | 30.79 |
| ATOM | 2161 | O | GLN | 287 | 21.120 | −5.957 | 63.229 | 1.00 | 29.31 |
| ATOM | 2162 | N | LEU | 288 | 20.921 | −6.791 | 61.158 | 1.00 | 29.33 |
| ATOM | 2163 | CA | LEU | 288 | 21.101 | −8.181 | 61.585 | 1.00 | 27.53 |
| ATOM | 2164 | CB | LEU | 288 | 20.940 | −9.129 | 60.393 | 1.00 | 28.13 |
| ATOM | 2165 | CG | LEU | 288 | 19.599 | −9.090 | 59.647 | 1.00 | 29.14 |
| ATOM | 2166 | CD1 | LEU | 288 | 19.390 | −10.418 | 58.922 | 1.00 | 27.60 |
| ATOM | 2167 | CD2 | LEU | 288 | 18.453 | −8.844 | 60.621 | 1.00 | 27.42 |
| ATOM | 2168 | C | LEU | 288 | 22.418 | −8.476 | 62.297 | 1.00 | 27.92 |
| ATOM | 2169 | O | LEU | 288 | 22.438 | −9.184 | 63.303 | 1.00 | 28.24 |
| ATOM | 2170 | N | TYR | 289 | 23.520 | −7.946 | 61.776 | 1.00 | 27.17 |
| ATOM | 2171 | CA | TYR | 289 | 24.819 | −8.153 | 62.399 | 1.00 | 24.83 |
| ATOM | 2172 | CB | TYR | 289 | 25.899 | −7.458 | 61.583 | 1.00 | 24.32 |
| ATOM | 2173 | CG | TYR | 289 | 27.303 | −7.575 | 62.137 | 1.00 | 21.26 |
| ATOM | 2174 | CD1 | TYR | 289 | 27.951 | −8.814 | 62.208 | 1.00 | 20.00 |
| ATOM | 2175 | CE1 | TYR | 289 | 29.281 | −8.909 | 62.616 | 1.00 | 18.43 |
| ATOM | 2176 | CD2 | TYR | 289 | 28.013 | −6.441 | 62.503 | 1.00 | 18.12 |
| ATOM | 2177 | CE2 | TYR | 289 | 29.338 | −6.520 | 62.918 | 1.00 | 20.65 |
| ATOM | 2178 | CZ | TYR | 289 | 29.976 | −7.762 | 62.966 | 1.00 | 21.27 |
| ATOM | 2179 | OH | TYR | 289 | 31.314 | −7.833 | 63.326 | 1.00 | 19.02 |
| ATOM | 2180 | C | TYR | 289 | 24.771 | −7.566 | 63.799 | 1.00 | 26.94 |
| ATOM | 2181 | O | TYR | 289 | 25.221 | −8.175 | 64.776 | 1.00 | 27.95 |
| ATOM | 2182 | N | GLU | 290 | 24.198 | −6.374 | 63.892 | 1.00 | 27.68 |
| ATOM | 2183 | CA | GLU | 290 | 24.078 | −5.686 | 65.165 | 1.00 | 26.41 |
| ATOM | 2184 | CB | GLU | 290 | 23.484 | −4.309 | 64.927 | 1.00 | 26.55 |
| ATOM | 2185 | CG | GLU | 290 | 23.059 | −3.595 | 66.180 | 1.00 | 27.05 |
| ATOM | 2186 | CD | GLU | 290 | 22.815 | −2.142 | 65.913 | 1.00 | 25.47 |
| ATOM | 2187 | OE1 | GLU | 290 | 23.716 | −1.336 | 66.204 | 1.00 | 27.17 |
| ATOM | 2188 | OE2 | GLU | 290 | 21.731 | −1.815 | 65.398 | 1.00 | 29.09 |
| ATOM | 2189 | C | GLU | 290 | 23.218 | −6.463 | 66.159 | 1.00 | 26.59 |
| ATOM | 2190 | O | GLU | 290 | 23.458 | −6.430 | 67.371 | 1.00 | 25.62 |
| ATOM | 2191 | N | LYS | 291 | 22.216 | −7.166 | 65.646 | 1.00 | 26.31 |
| ATOM | 2192 | CA | LYS | 291 | 21.343 | −7.942 | 66.509 | 1.00 | 27.77 |
| ATOM | 2193 | CB | LYS | 291 | 20.110 | −8.394 | 65.722 | 1.00 | 28.30 |
| ATOM | 2194 | CG | LYS | 291 | 19.096 | −7.263 | 65.585 | 1.00 | 33.35 |
| ATOM | 2195 | CD | LYS | 291 | 18.005 | −7.529 | 64.555 | 1.00 | 33.56 |
| ATOM | 2196 | CE | LYS | 291 | 17.038 | −6.330 | 64.522 | 1.00 | 36.46 |
| ATOM | 2197 | NZ | LYS | 291 | 16.150 | −6.319 | 63.327 | 1.00 | 36.55 |
| ATOM | 2198 | C | LYS | 291 | 22.073 | −9.123 | 67.138 | 1.00 | 26.53 |
| ATOM | 2199 | O | LYS | 291 | 21.584 | −9.736 | 68.084 | 1.00 | 27.81 |
| ATOM | 2200 | N | LEU | 292 | 23.261 | −9.426 | 66.628 | 1.00 | 26.02 |
| ATOM | 2201 | CA | LEU | 292 | 24.043 | −10.523 | 67.168 | 1.00 | 25.35 |
| ATOM | 2202 | CB | LEU | 292 | 24.922 | −11.140 | 66.079 | 1.00 | 25.16 |
| ATOM | 2203 | CG | LEU | 292 | 24.229 | −11.746 | 64.856 | 1.00 | 26.25 |
| ATOM | 2204 | CD1 | LEU | 292 | 25.274 | −12.190 | 63.827 | 1.00 | 23.09 |
| ATOM | 2205 | CD2 | LEU | 292 | 23.359 | −12.912 | 65.297 | 1.00 | 24.40 |
| ATOM | 2206 | C | LEU | 292 | 24.942 | −10.030 | 68.283 | 1.00 | 25.18 |
| ATOM | 2207 | O | LEU | 292 | 25.392 | −10.808 | 69.120 | 1.00 | 23.84 |
| ATOM | 2208 | N | ILE | 293 | 25.179 | −8.723 | 68.308 | 1.00 | 24.94 |
| ATOM | 2209 | CA | ILE | 293 | 26.107 | −8.140 | 69.267 | 1.00 | 23.59 |
| ATOM | 2210 | CB | ILE | 293 | 27.259 | −7.468 | 68.476 | 1.00 | 24.66 |
| ATOM | 2211 | CG2 | ILE | 293 | 28.233 | −6.762 | 69.409 | 1.00 | 21.05 |
| ATOM | 2212 | CG1 | ILE | 293 | 27.952 | −8.527 | 67.618 | 1.00 | 24.42 |
| ATOM | 2213 | CD1 | ILE | 293 | 28.715 | −7.965 | 66.441 | 1.00 | 25.64 |
| ATOM | 2214 | C | ILE | 293 | 25.560 | −7.148 | 70.278 | 1.00 | 25.10 |
| ATOM | 2215 | O | ILE | 293 | 25.797 | −7.289 | 71.474 | 1.00 | 23.79 |
| ATOM | 2216 | N | GLY | 294 | 24.845 | −6.136 | 69.781 | 1.00 | 28.83 |
| ATOM | 2217 | CA | GLY | 294 | 24.302 | −5.071 | 70.615 | 1.00 | 26.73 |
| ATOM | 2218 | C | GLY | 294 | 23.551 | −5.379 | 71.898 | 1.00 | 29.79 |
| ATOM | 2219 | O | GLY | 294 | 22.757 | −6.318 | 71.964 | 1.00 | 27.85 |
| ATOM | 2220 | N | GLY | 295 | 23.794 | −4.553 | 72.918 | 1.00 | 30.56 |
| ATOM | 2221 | CA | GLY | 295 | 23.136 | −4.722 | 74.204 | 1.00 | 33.01 |
| ATOM | 2222 | C | GLY | 295 | 21.628 | −4.539 | 74.144 | 1.00 | 34.05 |
| ATOM | 2223 | O | GLY | 295 | 20.927 | −4.810 | 75.107 | 1.00 | 34.93 |
| ATOM | 2224 | N | LYS | 296 | 21.124 | −4.058 | 73.016 | 1.00 | 35.19 |
| ATOM | 2225 | CA | LYS | 296 | 19.690 | −3.868 | 72.851 | 1.00 | 36.24 |
| ATOM | 2226 | CB | LYS | 296 | 19.419 | −2.988 | 71.626 | 1.00 | 38.05 |
| ATOM | 2227 | CG | LYS | 296 | 17.961 | −2.910 | 71.181 | 1.00 | 40.26 |
| ATOM | 2228 | CD | LYS | 296 | 17.122 | −2.093 | 72.141 | 1.00 | 43.32 |
| ATOM | 2229 | CE | LYS | 296 | 15.730 | −1.862 | 71.579 | 1.00 | 44.42 |
| ATOM | 2230 | NZ | LYS | 296 | 14.842 | −1.175 | 72.562 | 1.00 | 44.77 |
| ATOM | 2231 | C | LYS | 296 | 19.045 | −5.235 | 72.654 | 1.00 | 36.63 |
| ATOM | 2232 | O | LYS | 296 | 17.867 | −5.420 | 72.963 | 1.00 | 38.56 |
| ATOM | 2233 | N | TYR | 297 | 19.836 | −6.193 | 72.168 | 1.00 | 34.63 |
| ATOM | 2234 | CA | TYR | 297 | 19.346 | −7.539 | 71.890 | 1.00 | 33.22 |
| ATOM | 2235 | CB | TYR | 297 | 19.487 | −7.810 | 70.389 | 1.00 | 34.65 |
| ATOM | 2236 | CG | TYR | 297 | 19.073 | −6.631 | 69.535 | 1.00 | 36.28 |
| ATOM | 2237 | CD1 | TYR | 297 | 20.010 | −5.677 | 69.125 | 1.00 | 34.21 |
| ATOM | 2238 | CE1 | TYR | 297 | 19.622 | −4.548 | 68.404 | 1.00 | 36.22 |
| ATOM | 2239 | CD2 | TYR | 297 | 17.732 | −6.431 | 69.195 | 1.00 | 34.24 |
| ATOM | 2240 | CE2 | TYR | 297 | 17.330 | −5.305 | 68.476 | 1.00 | 35.71 |
| ATOM | 2241 | CZ | TYR | 297 | 18.280 | −4.368 | 68.082 | 1.00 | 37.38 |
| ATOM | 2242 | OH | TYR | 297 | 17.887 | −3.258 | 67.375 | 1.00 | 35.33 |
| ATOM | 2243 | C | TYR | 297 | 19.968 | −8.713 | 72.670 | 1.00 | 33.21 |
| ATOM | 2244 | O | TYR | 297 | 19.392 | −9.800 | 72.716 | 1.00 | 33.78 |
| ATOM | 2245 | N | MET | 298 | 21.126 | −8.504 | 73.283 | 1.00 | 31.19 |
| ATOM | 2246 | CA | MET | 298 | 21.803 | −9.576 | 74.005 | 1.00 | 30.16 |
| ATOM | 2247 | CB | MET | 298 | 23.075 | −9.038 | 74.644 | 1.00 | 30.05 |
| ATOM | 2248 | CG | MET | 298 | 23.957 | −10.104 | 75.231 | 1.00 | 26.86 |
| ATOM | 2249 | SD | MET | 298 | 25.486 | −9.405 | 75.850 | 1.00 | 32.83 |
| ATOM | 2250 | CE | MET | 298 | 26.409 | −9.201 | 74.338 | 1.00 | 29.59 |
| ATOM | 2251 | C | MET | 298 | 20.963 | −10.296 | 75.066 | 1.00 | 31.27 |
| ATOM | 2252 | O | MET | 298 | 20.882 | −11.529 | 75.077 | 1.00 | 29.78 |
| ATOM | 2253 | N | GLY | 299 | 20.353 | −9.530 | 75.963 | 1.00 | 30.40 |
| ATOM | 2254 | CA | GLY | 299 | 19.534 | −10.132 | 76.998 | 1.00 | 31.32 |
| ATOM | 2255 | C | GLY | 299 | 18.354 | −10.869 | 76.393 | 1.00 | 33.32 |
| ATOM | 2256 | O | GLY | 299 | 17.988 | −11.962 | 76.831 | 1.00 | 33.97 |
| ATOM | 2257 | N | GLU | 300 | 17.752 | −10.265 | 75.377 | 1.00 | 31.78 |
| ATOM | 2258 | CA | GLU | 300 | 16.617 | −10.874 | 74.707 | 1.00 | 31.93 |
| ATOM | 2259 | CB | GLU | 300 | 16.080 | −9.937 | 73.621 | 1.00 | 29.00 |
| ATOM | 2260 | CG | GLU | 300 | 14.877 | −10.486 | 72.881 | 1.00 | 32.60 |
| ATOM | 2261 | CD | GLU | 300 | 13.655 | −10.655 | 73.769 | 1.00 | 31.13 |
| ATOM | 2262 | OE1 | GLU | 300 | 12.629 | −11.144 | 73.265 | 1.00 | 34.55 |
| ATOM | 2263 | OE2 | GLU | 300 | 13.714 | −10.299 | 74.963 | 1.00 | 33.16 |
| ATOM | 2264 | C | GLU | 300 | 17.013 | −12.215 | 74.092 | 1.00 | 30.90 |
| ATOM | 2265 | O | GLU | 300 | 16.225 | −13.156 | 74.090 | 1.00 | 32.89 |
| ATOM | 2266 | N | LEU | 301 | 18.234 | −12.301 | 73.570 | 1.00 | 31.16 |
| ATOM | 2267 | CA | LEU | 301 | 18.714 | −13.546 | 72.973 | 1.00 | 28.93 |
| ATOM | 2268 | CB | LEU | 301 | 20.085 | −13.339 | 72.325 | 1.00 | 24.69 |
| ATOM | 2269 | CG | LEU | 301 | 20.152 | −12.667 | 70.952 | 1.00 | 24.17 |
| ATOM | 2270 | CD1 | LEU | 301 | 21.607 | −12.326 | 70.628 | 1.00 | 23.70 |
| ATOM | 2271 | CD2 | LEU | 301 | 19.560 | −13.598 | 69.886 | 1.00 | 23.13 |
| ATOM | 2272 | C | LEU | 301 | 18.814 | −14.616 | 74.056 | 1.00 | 29.42 |
| ATOM | 2273 | O | LEU | 301 | 18.408 | −15.761 | 73.853 | 1.00 | 32.03 |
| ATOM | 2274 | N | VAL | 302 | 19.365 | −14.239 | 75.204 | 1.00 | 28.73 |
| ATOM | 2275 | CA | VAL | 302 | 19.505 | −15.164 | 76.317 | 1.00 | 29.42 |
| ATOM | 2276 | CB | VAL | 302 | 20.265 | −14.510 | 77.497 | 1.00 | 26.51 |
| ATOM | 2277 | CG1 | VAL | 302 | 20.172 | −15.395 | 78.740 | 1.00 | 25.63 |
| ATOM | 2278 | CG2 | VAL | 302 | 21.731 | −14.301 | 77.117 | 1.00 | 25.98 |
| ATOM | 2279 | C | VAL | 302 | 18.127 | −15.624 | 76.795 | 1.00 | 31.88 |
| ATOM | 2280 | O | VAL | 302 | 17.934 | −16.795 | 77.112 | 1.00 | 32.71 |
| ATOM | 2281 | N | ARG | 303 | 17.171 | −14.703 | 76.835 | 1.00 | 32.91 |
| ATOM | 2282 | CA | ARG | 303 | 15.818 | −15.039 | 77.270 | 1.00 | 36.08 |
| ATOM | 2283 | CB | ARG | 303 | 14.910 | −13.802 | 77.250 | 1.00 | 35.86 |
| ATOM | 2284 | CG | ARG | 303 | 13.524 | −14.055 | 77.847 | 1.00 | 36.97 |
| ATOM | 2285 | CD | ARG | 303 | 12.660 | −12.802 | 77.833 | 1.00 | 39.15 |
| ATOM | 2286 | NE | ARG | 303 | 12.105 | −12.529 | 76.511 | 1.00 | 41.95 |
| ATOM | 2287 | CZ | ARG | 303 | 11.090 | −13.197 | 75.968 | 1.00 | 43.84 |
| ATOM | 2288 | NH1 | ARG | 303 | 10.502 | −14.182 | 76.631 | 1.00 | 42.47 |
| ATOM | 2289 | NH2 | ARG | 303 | 10.666 | −12.885 | 74.750 | 1.00 | 43.86 |
| ATOM | 2290 | C | ARG | 303 | 15.215 | −16.110 | 76.373 | 1.00 | 36.97 |
| ATOM | 2291 | O | ARG | 303 | 14.554 | −17.032 | 76.851 | 1.00 | 37.22 |
| ATOM | 2292 | N | LEU | 304 | 15.432 | −15.970 | 75.068 | 1.00 | 37.86 |
| ATOM | 2293 | CA | LEU | 304 | 14.914 | −16.924 | 74.103 | 1.00 | 37.63 |
| ATOM | 2294 | CB | LEU | 304 | 15.113 | −16.387 | 72.687 | 1.00 | 38.69 |
| ATOM | 2295 | CG | LEU | 304 | 13.944 | −15.590 | 72.104 | 1.00 | 40.35 |
| ATOM | 2296 | CD1 | LEU | 304 | 13.486 | −14.516 | 73.062 | 1.00 | 40.85 |
| ATOM | 2297 | CD2 | LEU | 304 | 14.378 | −14.986 | 70.785 | 1.00 | 42.07 |
| ATOM | 2298 | C | LEU | 304 | 15.602 | −18.272 | 74.262 | 1.00 | 37.69 |
| ATOM | 2299 | O | LEU | 304 | 14.978 | −19.324 | 74.120 | 1.00 | 38.84 |
| ATOM | 2300 | N | VAL | 305 | 16.893 | −18.238 | 74.558 | 1.00 | 36.28 |
| ATOM | 2301 | CA | VAL | 305 | 17.647 | −19.466 | 74.753 | 1.00 | 34.31 |
| ATOM | 2302 | CB | VAL | 305 | 19.148 | −19.184 | 74.908 | 1.00 | 32.24 |
| ATOM | 2303 | CG1 | VAL | 305 | 19.868 | −20.438 | 75.390 | 1.00 | 28.85 |
| ATOM | 2304 | CG2 | VAL | 305 | 19.717 | −18.713 | 73.578 | 1.00 | 29.80 |
| ATOM | 2305 | C | VAL | 305 | 17.153 | −20.158 | 76.012 | 1.00 | 35.48 |
| ATOM | 2306 | O | VAL | 305 | 17.079 | −21.389 | 76.070 | 1.00 | 34.47 |
| ATOM | 2307 | N | LEU | 306 | 16.820 | −19.362 | 77.023 | 1.00 | 34.14 |
| ATOM | 2308 | CA | LEU | 306 | 16.328 | −19.921 | 78.273 | 1.00 | 35.52 |
| ATOM | 2309 | CB | LEU | 306 | 16.257 | −18.841 | 79.353 | 1.00 | 32.11 |
| ATOM | 2310 | CG | LEU | 306 | 17.601 | −18.289 | 79.829 | 1.00 | 32.53 |
| ATOM | 2311 | CD1 | LEU | 306 | 17.359 | −17.326 | 80.964 | 1.00 | 33.54 |
| ATOM | 2312 | CD2 | LEU | 306 | 18.515 | −19.420 | 80.287 | 1.00 | 30.60 |
| ATOM | 2313 | C | LEU | 306 | 14.948 | −20.532 | 78.049 | 1.00 | 37.53 |
| ATOM | 2314 | O | LEU | 306 | 14.637 | −21.608 | 78.566 | 1.00 | 33.87 |
| ATOM | 2315 | N | LEU | 307 | 14.129 | −19.850 | 77.257 | 1.00 | 39.39 |
| ATOM | 2316 | CA | LEU | 307 | 12.787 | −20.336 | 76.971 | 1.00 | 41.43 |
| ATOM | 2317 | CB | LEU | 307 | 12.011 | −19.296 | 76.165 | 1.00 | 40.84 |
| ATOM | 2318 | CG | LEU | 307 | 10.932 | −18.527 | 76.935 | 1.00 | 43.43 |
| ATOM | 2319 | CD1 | LEU | 307 | 11.389 | −18.243 | 78.356 | 1.00 | 43.36 |
| ATOM | 2320 | CD2 | LEU | 307 | 10.610 | −17.233 | 76.197 | 1.00 | 41.75 |
| ATOM | 2321 | C | LEU | 307 | 12.802 | −21.674 | 76.239 | 1.00 | 42.39 |
| ATOM | 2322 | O | LEU | 307 | 11.974 | −22.537 | 76.514 | 1.00 | 42.90 |
| ATOM | 2323 | N | ARG | 308 | 13.729 | −21.860 | 75.306 | 1.00 | 42.02 |
| ATOM | 2324 | CA | ARG | 308 | 13.771 | −23.132 | 74.605 | 1.00 | 42.88 |
| ATOM | 2325 | CB | ARG | 308 | 14.765 | −23.125 | 73.445 | 1.00 | 43.55 |
| ATOM | 2326 | CG | ARG | 308 | 14.891 | −24.514 | 72.837 | 1.00 | 47.00 |
| ATOM | 2327 | CD | ARG | 308 | 15.908 | −24.626 | 71.729 | 1.00 | 49.25 |
| ATOM | 2328 | NE | ARG | 308 | 16.079 | −26.026 | 71.349 | 1.00 | 52.10 |
| ATOM | 2329 | CZ | ARG | 308 | 16.915 | −26.456 | 70.410 | 1.00 | 52.45 |
| ATOM | 2330 | NH1 | ARG | 308 | 17.663 | −25.591 | 69.739 | 1.00 | 54.77 |
| ATOM | 2331 | NH2 | ARG | 308 | 17.016 | −27.756 | 70.154 | 1.00 | 51.73 |
| ATOM | 2332 | C | ARG | 308 | 14.181 | −24.222 | 75.582 | 1.00 | 43.27 |
| ATOM | 2333 | O | ARG | 308 | 13.654 | −25.333 | 75.540 | 1.00 | 42.09 |
| ATOM | 2334 | N | LEU | 309 | 15.135 | −23.895 | 76.452 | 1.00 | 42.54 |
| ATOM | 2335 | CA | LEU | 309 | 15.627 | −24.837 | 77.447 | 1.00 | 42.29 |
| ATOM | 2336 | CB | LEU | 309 | 16.771 | −24.207 | 78.248 | 1.00 | 40.55 |
| ATOM | 2337 | CG | LEU | 309 | 18.193 | −24.656 | 77.886 | 1.00 | 39.65 |
| ATOM | 2338 | CD1 | LEU | 309 | 18.313 | −24.973 | 76.416 | 1.00 | 38.56 |
| ATOM | 2339 | CD2 | LEU | 309 | 19.171 | −23.569 | 78.284 | 1.00 | 37.67 |
| ATOM | 2340 | C | LEU | 309 | 14.515 | −25.302 | 78.379 | 1.00 | 42.66 |
| ATOM | 2341 | O | LEU | 309 | 14.509 | −26.450 | 78.818 | 1.00 | 41.33 |
| ATOM | 2342 | N | VAL | 310 | 13.570 | −24.416 | 78.676 | 1.00 | 44.27 |
| ATOM | 2343 | CA | VAL | 310 | 12.464 | −24.789 | 79.543 | 1.00 | 46.40 |
| ATOM | 2344 | CB | VAL | 310 | 11.711 | −23.546 | 80.111 | 1.00 | 46.06 |
| ATOM | 2345 | CG1 | VAL | 310 | 12.682 | −22.613 | 80.807 | 1.00 | 45.43 |
| ATOM | 2346 | CG2 | VAL | 310 | 10.976 | −22.825 | 79.014 | 1.00 | 48.29 |
| ATOM | 2347 | C | VAL | 310 | 11.479 | −25.666 | 78.769 | 1.00 | 48.00 |
| ATOM | 2348 | O | VAL | 310 | 10.952 | −26.638 | 79.311 | 1.00 | 47.71 |
| ATOM | 2349 | N | ASP | 311 | 11.242 | −25.333 | 77.501 | 1.00 | 49.58 |
| ATOM | 2350 | CA | ASP | 311 | 10.313 | −26.104 | 76.683 | 1.00 | 52.37 |
| ATOM | 2351 | CB | ASP | 311 | 9.978 | −25.365 | 75.382 | 1.00 | 54.70 |
| ATOM | 2352 | CG | ASP | 311 | 9.318 | −24.014 | 75.626 | 1.00 | 58.89 |
| ATOM | 2353 | OD1 | ASP | 311 | 8.742 | −23.808 | 76.719 | 1.00 | 60.74 |
| ATOM | 2354 | OD2 | ASP | 311 | 9.364 | −23.158 | 74.713 | 1.00 | 60.54 |
| ATOM | 2355 | C | ASP | 311 | 10.872 | −27.485 | 76.365 | 1.00 | 52.35 |
| ATOM | 2356 | O | ASP | 311 | 10.131 | −28.388 | 75.982 | 1.00 | 55.07 |
| ATOM | 2357 | N | GLU | 312 | 12.180 | −27.642 | 76.515 | 1.00 | 51.23 |
| ATOM | 2358 | CA | GLU | 312 | 12.828 | −28.926 | 76.279 | 1.00 | 51.12 |
| ATOM | 2359 | CB | GLU | 312 | 14.277 | −28.729 | 75.834 | 1.00 | 52.62 |
| ATOM | 2360 | CG | GLU | 312 | 14.445 | −28.141 | 74.448 | 1.00 | 57.13 |
| ATOM | 2361 | CD | GLU | 312 | 14.187 | −29.153 | 73.358 | 1.00 | 58.40 |
| ATOM | 2362 | OE1 | GLU | 312 | 14.831 | −30.222 | 73.385 | 1.00 | 59.31 |
| ATOM | 2363 | OE2 | GLU | 312 | 13.346 | −28.879 | 72.476 | 1.00 | 60.41 |
| ATOM | 2364 | C | GLU | 312 | 12.810 | −29.660 | 77.611 | 1.00 | 50.76 |
| ATOM | 2365 | O | GLU | 312 | 13.292 | −30.787 | 77.720 | 1.00 | 50.64 |
| ATOM | 2366 | N | ASN | 313 | 12.265 | −28.989 | 78.624 | 1.00 | 50.08 |
| ATOM | 2367 | CA | ASN | 313 | 12.154 | −29.533 | 79.974 | 1.00 | 51.37 |
| ATOM | 2368 | CB | ASN | 313 | 11.428 | −30.886 | 79.932 | 1.00 | 53.51 |
| ATOM | 2369 | CG | ASN | 313 | 10.846 | −31.275 | 81.271 | 1.00 | 55.73 |
| ATOM | 2370 | OD1 | ASN | 313 | 10.011 | −30.560 | 81.824 | 1.00 | 58.95 |
| ATOM | 2371 | ND2 | ASN | 313 | 11.281 | −32.415 | 81.803 | 1.00 | 59.16 |
| ATOM | 2372 | C | ASN | 313 | 13.524 | −29.693 | 80.635 | 1.00 | 50.00 |
| ATOM | 2373 | O | ASN | 313 | 13.733 | −30.595 | 81.447 | 1.00 | 50.40 |
| ATOM | 2374 | N | LEU | 314 | 14.449 | −28.799 | 80.296 | 1.00 | 48.35 |
| ATOM | 2375 | CA | LEU | 314 | 15.805 | −28.843 | 80.835 | 1.00 | 45.12 |
| ATOM | 2376 | CB | LEU | 314 | 16.819 | −28.785 | 79.688 | 1.00 | 44.25 |
| ATOM | 2377 | CG | LEU | 314 | 16.759 | −29.872 | 78.611 | 1.00 | 45.98 |
| ATOM | 2378 | CD1 | LEU | 314 | 17.619 | −29.465 | 77.416 | 1.00 | 43.63 |
| ATOM | 2379 | CD2 | LEU | 314 | 17.232 | −31.201 | 79.196 | 1.00 | 45.09 |
| ATOM | 2380 | C | LEU | 314 | 16.119 | −27.724 | 81.829 | 1.00 | 43.38 |
| ATOM | 2381 | O | LEU | 314 | 17.180 | −27.732 | 82.449 | 1.00 | 41.90 |
| ATOM | 2382 | N | LEU | 315 | 15.211 | −26.765 | 81.982 | 1.00 | 41.74 |
| ATOM | 2383 | CA | LEU | 315 | 15.446 | −25.645 | 82.899 | 1.00 | 42.39 |
| ATOM | 2384 | CB | LEU | 315 | 15.907 | −24.407 | 82.116 | 1.00 | 40.17 |
| ATOM | 2385 | CG | LEU | 315 | 17.243 | −23.721 | 82.428 | 1.00 | 39.81 |
| ATOM | 2386 | CD1 | LEU | 315 | 17.262 | −22.383 | 81.689 | 1.00 | 41.89 |
| ATOM | 2387 | CD2 | LEU | 315 | 17.421 | −23.482 | 83.920 | 1.00 | 37.58 |
| ATOM | 2388 | C | LEU | 315 | 14.198 | −25.278 | 83.694 | 1.00 | 42.28 |
| ATOM | 2389 | O | LEU | 315 | 13.103 | −25.214 | 83.144 | 1.00 | 40.83 |
| ATOM | 2390 | N | PHE | 316 | 14.377 | −25.021 | 84.986 | 1.00 | 43.70 |
| ATOM | 2391 | CA | PHE | 316 | 13.271 | −24.648 | 85.863 | 1.00 | 46.70 |
| ATOM | 2392 | CB | PHE | 316 | 12.717 | −23.278 | 85.459 | 1.00 | 47.06 |
| ATOM | 2393 | CG | PHE | 316 | 13.776 | −22.247 | 85.187 | 1.00 | 47.07 |
| ATOM | 2394 | CD1 | PHE | 316 | 14.824 | −22.051 | 86.082 | 1.00 | 47.24 |
| ATOM | 2395 | CD2 | PHE | 316 | 13.722 | −21.467 | 84.037 | 1.00 | 47.25 |
| ATOM | 2396 | CE1 | PHE | 316 | 15.803 | −21.094 | 85.835 | 1.00 | 46.12 |
| ATOM | 2397 | CE2 | PHE | 316 | 14.695 | −20.507 | 83.782 | 1.00 | 47.70 |
| ATOM | 2398 | CZ | PHE | 316 | 15.738 | −20.321 | 84.683 | 1.00 | 47.68 |
| ATOM | 2399 | C | PHE | 316 | 12.131 | −25.672 | 85.857 | 1.00 | 48.45 |
| ATOM | 2400 | O | PHE | 316 | 10.960 | −25.306 | 85.967 | 1.00 | 48.86 |
| ATOM | 2401 | N | HIS | 317 | 12.473 | −26.950 | 85.725 | 1.00 | 50.80 |
| ATOM | 2402 | CA | HIS | 317 | 11.469 | −28.009 | 85.712 | 1.00 | 53.83 |
| ATOM | 2403 | CB | HIS | 317 | 10.655 | −27.986 | 87.010 | 1.00 | 57.67 |
| ATOM | 2404 | CG | HIS | 317 | 11.496 | −27.985 | 88.246 | 1.00 | 61.10 |
| ATOM | 2405 | CD2 | HIS | 317 | 11.558 | −27.116 | 89.282 | 1.00 | 63.07 |
| ATOM | 2406 | ND1 | HIS | 317 | 12.430 | −28.965 | 88.509 | 1.00 | 62.35 |
| ATOM | 2407 | CE1 | HIS | 317 | 13.032 | −28.699 | 89.655 | 1.00 | 64.77 |
| ATOM | 2408 | NE2 | HIS | 317 | 12.521 | −27.582 | 90.144 | 1.00 | 65.99 |
| ATOM | 2409 | C | HIS | 317 | 10.521 | −27.859 | 84.534 | 1.00 | 53.57 |
| ATOM | 2410 | O | HIS | 317 | 9.429 | −28.425 | 84.537 | 1.00 | 53.60 |
| ATOM | 2411 | N | GLY | 318 | 10.939 | −27.090 | 83.534 | 1.00 | 52.50 |
| ATOM | 2412 | CA | GLY | 318 | 10.113 | −26.881 | 82.358 | 1.00 | 51.83 |
| ATOM | 2413 | C | GLY | 318 | 8.940 | −25.958 | 82.615 | 1.00 | 51.72 |
| ATOM | 2414 | O | GLY | 318 | 7.939 | −25.999 | 81.904 | 1.00 | 50.88 |
| ATOM | 2415 | N | GLU | 319 | 9.073 | −25.110 | 83.627 | 1.00 | 53.43 |
| ATOM | 2416 | CA | GLU | 319 | 8.014 | −24.182 | 83.996 | 1.00 | 55.73 |
| ATOM | 2417 | CB | GLU | 319 | 7.510 | −24.544 | 85.392 | 1.00 | 58.85 |
| ATOM | 2418 | CG | GLU | 319 | 6.145 | −23.998 | 85.761 | 1.00 | 63.60 |
| ATOM | 2419 | CD | GLU | 319 | 5.590 | −24.664 | 87.016 | 1.00 | 66.32 |
| ATOM | 2420 | OE1 | GLU | 319 | 6.206 | −24.527 | 88.100 | 1.00 | 65.47 |
| ATOM | 2421 | OE2 | GLU | 319 | 4.540 | −25.335 | 86.913 | 1.00 | 67.45 |
| ATOM | 2422 | C | GLU | 319 | 8.538 | −22.748 | 83.966 | 1.00 | 55.18 |
| ATOM | 2423 | O | GLU | 319 | 9.278 | −22.324 | 84.851 | 1.00 | 55.23 |
| ATOM | 2424 | N | ALA | 320 | 8.145 | −22.006 | 82.938 | 1.00 | 55.14 |
| ATOM | 2425 | CA | ALA | 320 | 8.585 | −20.630 | 82.780 | 1.00 | 55.95 |
| ATOM | 2426 | CB | ALA | 320 | 8.609 | −20.265 | 81.304 | 1.00 | 55.13 |
| ATOM | 2427 | C | ALA | 320 | 7.708 | −19.649 | 83.544 | 1.00 | 56.88 |
| ATOM | 2428 | O | ALA | 320 | 6.487 | −19.789 | 83.584 | 1.00 | 58.58 |
| ATOM | 2429 | N | SER | 321 | 8.344 | −18.648 | 84.141 | 1.00 | 57.00 |
| ATOM | 2430 | CA | SER | 321 | 7.644 | −17.625 | 84.902 | 1.00 | 56.57 |
| ATOM | 2431 | CB | SER | 321 | 8.649 | −16.808 | 85.705 | 1.00 | 56.74 |
| ATOM | 2432 | OG | SER | 321 | 8.013 | −15.725 | 86.349 | 1.00 | 57.41 |
| ATOM | 2433 | C | SER | 321 | 6.853 | −16.689 | 83.995 | 1.00 | 58.61 |
| ATOM | 2434 | O | SER | 321 | 7.054 | −16.665 | 82.783 | 1.00 | 58.41 |
| ATOM | 2435 | N | GLU | 322 | 5.955 | −15.914 | 84.595 | 1.00 | 60.41 |
| ATOM | 2436 | CA | GLU | 322 | 5.133 | −14.960 | 83.858 | 1.00 | 62.09 |
| ATOM | 2437 | CB | GLU | 322 | 4.171 | −14.254 | 84.819 | 1.00 | 65.34 |
| ATOM | 2438 | CG | GLU | 322 | 3.185 | −13.299 | 84.165 | 1.00 | 69.70 |
| ATOM | 2439 | CD | GLU | 322 | 2.075 | −14.020 | 83.418 | 1.00 | 73.68 |
| ATOM | 2440 | OE1 | GLU | 322 | 1.379 | −14.851 | 84.046 | 1.00 | 74.78 |
| ATOM | 2441 | OE2 | GLU | 322 | 1.896 | −13.751 | 82.208 | 1.00 | 75.02 |
| ATOM | 2442 | C | GLU | 322 | 6.047 | −13.929 | 83.204 | 1.00 | 61.24 |
| ATOM | 2443 | O | GLU | 322 | 5.913 | −13.612 | 82.022 | 1.00 | 60.81 |
| ATOM | 2444 | N | GLN | 323 | 6.987 | −13.420 | 83.991 | 1.00 | 60.42 |
| ATOM | 2445 | CA | GLN | 323 | 7.935 | −12.422 | 83.521 | 1.00 | 58.63 |
| ATOM | 2446 | CB | GLN | 323 | 8.729 | −11.863 | 84.700 | 1.00 | 59.77 |
| ATOM | 2447 | CG | GLN | 323 | 7.902 | −11.039 | 85.658 | 1.00 | 61.20 |
| ATOM | 2448 | CD | GLN | 323 | 8.690 | −10.608 | 86.873 | 1.00 | 63.03 |
| ATOM | 2449 | OE1 | GLN | 323 | 9.672 | −9.866 | 86.767 | 1.00 | 63.70 |
| ATOM | 2450 | NE2 | GLN | 323 | 8.266 | −11.074 | 88.044 | 1.00 | 64.05 |
| ATOM | 2451 | C | GLN | 323 | 8.904 | −12.955 | 82.478 | 1.00 | 56.96 |
| ATOM | 2452 | O | GLN | 323 | 9.244 | −12.255 | 81.526 | 1.00 | 56.89 |
| ATOM | 2453 | N | LEU | 324 | 9.351 | −14.190 | 82.652 | 1.00 | 53.93 |
| ATOM | 2454 | CA | LEU | 324 | 10.298 | −14.763 | 81.713 | 1.00 | 52.62 |
| ATOM | 2455 | CB | LEU | 324 | 10.745 | −16.151 | 82.180 | 1.00 | 51.22 |
| ATOM | 2456 | CG | LEU | 324 | 11.830 | −16.826 | 81.334 | 1.00 | 50.58 |
| ATOM | 2457 | CD1 | LEU | 324 | 13.076 | −15.952 | 81.299 | 1.00 | 49.50 |
| ATOM | 2458 | CD2 | LEU | 324 | 12.160 | −18.192 | 81.909 | 1.00 | 49.35 |
| ATOM | 2459 | C | LEU | 324 | 9.730 | −14.855 | 80.306 | 1.00 | 52.38 |
| ATOM | 2460 | O | LEU | 324 | 10.485 | −14.870 | 79.337 | 1.00 | 51.83 |
| ATOM | 2461 | N | ARG | 325 | 8.405 | −14.902 | 80.193 | 1.00 | 52.63 |
| ATOM | 2462 | CA | ARG | 325 | 7.759 | −15.015 | 78.887 | 1.00 | 53.00 |
| ATOM | 2463 | CB | ARG | 325 | 6.477 | −15.848 | 79.000 | 1.00 | 54.77 |
| ATOM | 2464 | CG | ARG | 325 | 6.585 | −17.005 | 79.985 | 1.00 | 58.57 |
| ATOM | 2465 | CD | ARG | 325 | 6.013 | −18.330 | 79.458 | 1.00 | 60.34 |
| ATOM | 2466 | NE | ARG | 325 | 6.881 | −18.961 | 78.464 | 1.00 | 62.28 |
| ATOM | 2467 | CZ | ARG | 325 | 6.953 | −20.273 | 78.249 | 1.00 | 62.81 |
| ATOM | 2468 | NH1 | ARG | 325 | 6.208 | −21.109 | 78.963 | 1.00 | 62.98 |
| ATOM | 2469 | NH2 | ARG | 325 | 7.769 | −20.752 | 77.317 | 1.00 | 62.50 |
| ATOM | 2470 | C | ARG | 325 | 7.430 | −13.663 | 78.266 | 1.00 | 52.20 |
| ATOM | 2471 | O | ARG | 325 | 6.835 | −13.595 | 77.194 | 1.00 | 51.65 |
| ATOM | 2472 | N | THR | 326 | 7.820 | −12.589 | 78.940 | 1.00 | 51.52 |
| ATOM | 2473 | CA | THR | 326 | 7.562 | −11.248 | 78.438 | 1.00 | 53.54 |
| ATOM | 2474 | CB | THR | 326 | 7.031 | −10.343 | 79.570 | 1.00 | 54.40 |
| ATOM | 2475 | OG1 | THR | 326 | 8.068 | −10.120 | 80.534 | 1.00 | 56.68 |
| ATOM | 2476 | CG2 | THR | 326 | 5.858 | −11.012 | 80.274 | 1.00 | 53.00 |
| ATOM | 2477 | C | THR | 326 | 8.853 | −10.655 | 77.850 | 1.00 | 54.00 |
| ATOM | 2478 | O | THR | 326 | 9.891 | −10.626 | 78.515 | 1.00 | 53.48 |
| ATOM | 2479 | N | ARG | 327 | 8.782 | −10.191 | 76.604 | 1.00 | 54.30 |
| ATOM | 2480 | CA | ARG | 327 | 9.948 | −9.628 | 75.923 | 1.00 | 55.25 |
| ATOM | 2481 | CB | ARG | 327 | 9.568 | −9.074 | 74.550 | 1.00 | 58.73 |
| ATOM | 2482 | CG | ARG | 327 | 9.050 | −10.101 | 73.572 | 1.00 | 62.94 |
| ATOM | 2483 | CD | ARG | 327 | 9.189 | −9.599 | 72.143 | 1.00 | 66.63 |
| ATOM | 2484 | NE | ARG | 327 | 8.462 | −10.454 | 71.213 | 1.00 | 70.25 |
| ATOM | 2485 | CZ | ARG | 327 | 7.136 | −10.522 | 71.154 | 1.00 | 72.29 |
| ATOM | 2486 | NH1 | ARG | 327 | 6.399 | −9.778 | 71.969 | 1.00 | 72.86 |
| ATOM | 2487 | NH2 | ARG | 327 | 6.546 | −11.338 | 70.288 | 1.00 | 73.24 |
| ATOM | 2488 | C | ARG | 327 | 10.660 | −8.529 | 76.688 | 1.00 | 53.79 |
| ATOM | 2489 | O | ARG | 327 | 10.027 | −7.690 | 77.326 | 1.00 | 55.10 |
| ATOM | 2490 | N | GLY | 328 | 11.986 | −8.535 | 76.604 | 1.00 | 50.97 |
| ATOM | 2491 | CA | GLY | 328 | 12.773 | −7.520 | 77.276 | 1.00 | 50.03 |
| ATOM | 2492 | C | GLY | 328 | 12.922 | −7.715 | 78.770 | 1.00 | 49.36 |
| ATOM | 2493 | O | GLY | 328 | 13.622 | −6.942 | 79.426 | 1.00 | 49.68 |
| ATOM | 2494 | N | ALA | 329 | 12.274 | −8.740 | 79.315 | 1.00 | 47.47 |
| ATOM | 2495 | CA | ALA | 329 | 12.354 | −9.007 | 80.749 | 1.00 | 46.93 |
| ATOM | 2496 | CB | ALA | 329 | 11.468 | −10.184 | 81.115 | 1.00 | 48.23 |
| ATOM | 2497 | C | ALA | 329 | 13.786 | −9.287 | 81.173 | 1.00 | 45.48 |
| ATOM | 2498 | O | ALA | 329 | 14.247 | −8.794 | 82.203 | 1.00 | 44.91 |
| ATOM | 2499 | N | PHE | 330 | 14.490 | −10.088 | 80.383 | 1.00 | 43.75 |
| ATOM | 2500 | CA | PHE | 330 | 15.870 | −10.392 | 80.710 | 1.00 | 42.95 |
| ATOM | 2501 | CB | PHE | 330 | 16.271 | −11.760 | 80.156 | 1.00 | 39.40 |
| ATOM | 2502 | CG | PHE | 330 | 17.478 | −12.350 | 80.829 | 1.00 | 36.90 |
| ATOM | 2503 | CD1 | PHE | 330 | 18.761 | −11.985 | 80.436 | 1.00 | 35.73 |
| ATOM | 2504 | CD2 | PHE | 330 | 17.330 | −13.241 | 81.893 | 1.00 | 35.23 |
| ATOM | 2505 | CE1 | PHE | 330 | 19.878 | −12.496 | 81.093 | 1.00 | 33.48 |
| ATOM | 2506 | CE2 | PHE | 330 | 18.443 | −13.759 | 82.558 | 1.00 | 31.61 |
| ATOM | 2507 | CZ | PHE | 330 | 19.716 | −13.387 | 82.160 | 1.00 | 33.39 |
| ATOM | 2508 | C | PHE | 330 | 16.752 | −9.292 | 80.130 | 1.00 | 43.51 |
| ATOM | 2509 | O | PHE | 330 | 17.202 | −9.373 | 78.986 | 1.00 | 44.11 |
| ATOM | 2510 | N | GLU | 331 | 16.962 | −8.254 | 80.935 | 1.00 | 43.95 |
| ATOM | 2511 | CA | GLU | 331 | 17.777 | −7.099 | 80.569 | 1.00 | 43.11 |
| ATOM | 2512 | CB | GLU | 331 | 17.767 | −6.068 | 81.697 | 1.00 | 46.19 |
| ATOM | 2513 | CG | GLU | 331 | 16.393 | −5.551 | 82.092 | 1.00 | 50.13 |
| ATOM | 2514 | CD | GLU | 331 | 16.458 | −4.651 | 83.316 | 1.00 | 53.54 |
| ATOM | 2515 | OE1 | GLU | 331 | 17.324 | −3.745 | 83.343 | 1.00 | 55.03 |
| ATOM | 2516 | OE2 | GLU | 331 | 15.646 | −4.846 | 84.247 | 1.00 | 53.56 |
| ATOM | 2517 | C | GLU | 331 | 19.216 | −7.511 | 80.310 | 1.00 | 42.02 |
| ATOM | 2518 | O | GLU | 331 | 19.742 | −8.411 | 80.968 | 1.00 | 42.05 |
| ATOM | 2519 | N | THR | 332 | 19.855 | −6.830 | 79.365 | 1.00 | 39.23 |
| ATOM | 2520 | CA | THR | 332 | 21.235 | −7.122 | 79.017 | 1.00 | 36.08 |
| ATOM | 2521 | CB | THR | 332 | 21.713 | −6.200 | 77.869 | 1.00 | 36.47 |
| ATOM | 2522 | OG1 | THR | 332 | 21.297 | −6.762 | 76.618 | 1.00 | 33.61 |
| ATOM | 2523 | CG2 | THR | 332 | 23.235 | −6.030 | 77.884 | 1.00 | 31.36 |
| ATOM | 2524 | C | THR | 332 | 22.159 | −6.987 | 80.219 | 1.00 | 35.73 |
| ATOM | 2525 | O | THR | 332 | 23.209 | −7.634 | 80.280 | 1.00 | 35.30 |
| ATOM | 2526 | N | ARG | 333 | 21.782 | −6.151 | 81.180 | 1.00 | 34.21 |
| ATOM | 2527 | CA | ARG | 333 | 22.632 | −6.003 | 82.353 | 1.00 | 34.18 |
| ATOM | 2528 | CB | ARG | 333 | 22.211 | −4.786 | 83.193 | 1.00 | 36.60 |
| ATOM | 2529 | CG | ARG | 333 | 20.830 | −4.854 | 83.835 | 1.00 | 39.58 |
| ATOM | 2530 | CD | ARG | 333 | 20.488 | −3.518 | 84.520 | 1.00 | 42.78 |
| ATOM | 2531 | NE | ARG | 333 | 19.264 | −3.590 | 85.316 | 1.00 | 45.29 |
| ATOM | 2532 | CZ | ARG | 333 | 19.205 | −4.039 | 86.567 | 1.00 | 47.32 |
| ATOM | 2533 | NH1 | ARG | 333 | 20.305 | −4.455 | 87.182 | 1.00 | 49.55 |
| ATOM | 2534 | NH2 | ARG | 333 | 18.042 | −4.080 | 87.205 | 1.00 | 48.70 |
| ATOM | 2535 | C | ARG | 333 | 22.609 | −7.298 | 83.181 | 1.00 | 31.65 |
| ATOM | 2536 | O | ARG | 333 | 23.584 | −7.625 | 83.863 | 1.00 | 31.61 |
| ATOM | 2537 | N | PHE | 334 | 21.513 | −8.049 | 83.105 | 1.00 | 31.01 |
| ATOM | 2538 | CA | PHE | 334 | 21.431 | −9.317 | 83.835 | 1.00 | 30.67 |
| ATOM | 2539 | CB | PHE | 334 | 20.048 | −9.967 | 83.678 | 1.00 | 30.39 |
| ATOM | 2540 | CG | PHE | 334 | 18.923 | −9.210 | 84.330 | 1.00 | 30.58 |
| ATOM | 2541 | CD1 | PHE | 334 | 19.170 | −8.214 | 85.269 | 1.00 | 29.37 |
| ATOM | 2542 | CD2 | PHE | 334 | 17.600 | −9.522 | 84.019 | 1.00 | 31.94 |
| ATOM | 2543 | CE1 | PHE | 334 | 18.113 | −7.539 | 85.891 | 1.00 | 31.67 |
| ATOM | 2544 | CE2 | PHE | 334 | 16.535 | −8.851 | 84.636 | 1.00 | 32.25 |
| ATOM | 2545 | CZ | PHE | 334 | 16.796 | −7.857 | 85.575 | 1.00 | 28.89 |
| ATOM | 2546 | C | PHE | 334 | 22.496 | −10.287 | 83.295 | 1.00 | 30.73 |
| ATOM | 2547 | O | PHE | 334 | 23.136 | −11.016 | 84.064 | 1.00 | 30.77 |
| ATOM | 2548 | N | VAL | 335 | 22.685 | −10.290 | 81.973 | 1.00 | 29.44 |
| ATOM | 2549 | CA | VAL | 335 | 23.672 | −11.165 | 81.350 | 1.00 | 30.61 |
| ATOM | 2550 | CB | VAL | 335 | 23.777 | −10.921 | 79.831 | 1.00 | 30.75 |
| ATOM | 2551 | CG1 | VAL | 335 | 24.774 | −11.898 | 79.216 | 1.00 | 32.48 |
| ATOM | 2552 | CG2 | VAL | 335 | 22.424 | −11.078 | 79.181 | 1.00 | 29.80 |
| ATOM | 2553 | C | VAL | 335 | 25.041 | −10.904 | 81.964 | 1.00 | 31.64 |
| ATOM | 2554 | O | VAL | 335 | 25.759 | −11.830 | 82.356 | 1.00 | 31.87 |
| ATOM | 2555 | N | SER | 336 | 25.382 | −9.623 | 82.048 | 1.00 | 33.23 |
| ATOM | 2556 | CA | SER | 336 | 26.655 | −9.173 | 82.593 | 1.00 | 32.42 |
| ATOM | 2557 | CB | SER | 336 | 26.778 | −7.660 | 82.384 | 1.00 | 33.94 |
| ATOM | 2558 | OG | SER | 336 | 28.080 | −7.204 | 82.682 | 1.00 | 38.27 |
| ATOM | 2559 | C | SER | 336 | 26.793 | −9.524 | 84.078 | 1.00 | 32.82 |
| ATOM | 2560 | O | SER | 336 | 27.863 | −9.917 | 84.529 | 1.00 | 33.76 |
| ATOM | 2561 | N | GLN | 337 | 25.711 | −9.389 | 84.839 | 1.00 | 32.64 |
| ATOM | 2562 | CA | GLN | 337 | 25.753 | −9.715 | 86.260 | 1.00 | 34.83 |
| ATOM | 2563 | CB | GLN | 337 | 24.480 | −9.233 | 86.958 | 1.00 | 37.43 |
| ATOM | 2564 | CG | GLN | 337 | 24.339 | −7.721 | 86.972 | 1.00 | 42.29 |
| ATOM | 2565 | CD | GLN | 337 | 22.984 | −7.260 | 87.471 | 1.00 | 44.59 |
| ATOM | 2566 | OE1 | GLN | 337 | 22.710 | −6.062 | 87.525 | 1.00 | 46.49 |
| ATOM | 2567 | NE2 | GLN | 337 | 22.128 | −8.209 | 87.835 | 1.00 | 43.79 |
| ATOM | 2568 | C | GLN | 337 | 25.899 | −11.217 | 86.447 | 1.00 | 33.66 |
| ATOM | 2569 | O | GLN | 337 | 26.663 | −11.674 | 87.297 | 1.00 | 35.28 |
| ATOM | 2570 | N | VAL | 338 | 25.159 | −11.983 | 85.655 | 1.00 | 31.29 |
| ATOM | 2571 | CA | VAL | 338 | 25.236 | −13.432 | 85.743 | 1.00 | 29.21 |
| ATOM | 2572 | CB | VAL | 338 | 24.326 | −14.102 | 84.690 | 1.00 | 28.27 |
| ATOM | 2573 | CG1 | VAL | 338 | 24.687 | −15.571 | 84.525 | 1.00 | 27.17 |
| ATOM | 2574 | CG2 | VAL | 338 | 22.877 | −13.984 | 85.129 | 1.00 | 26.99 |
| ATOM | 2575 | C | VAL | 338 | 26.678 | −13.877 | 85.547 | 1.00 | 27.35 |
| ATOM | 2576 | O | VAL | 338 | 27.176 | −14.722 | 86.284 | 1.00 | 26.69 |
| ATOM | 2577 | N | GLU | 339 | 27.361 | −13.283 | 84.576 | 1.00 | 27.29 |
| ATOM | 2578 | CA | GLU | 339 | 28.747 | −13.657 | 84.314 | 1.00 | 27.15 |
| ATOM | 2579 | CB | GLU | 339 | 29.136 | −13.303 | 82.871 | 1.00 | 27.02 |
| ATOM | 2580 | CG | GLU | 339 | 28.404 | −14.185 | 81.843 | 1.00 | 30.73 |
| ATOM | 2581 | CD | GLU | 339 | 28.942 | −14.063 | 80.425 | 1.00 | 30.33 |
| ATOM | 2582 | OE1 | GLU | 339 | 30.121 | −14.414 | 80.185 | 1.00 | 34.73 |
| ATOM | 2583 | OE2 | GLU | 339 | 28.179 | −13.619 | 79.548 | 1.00 | 29.50 |
| ATOM | 2584 | C | GLU | 339 | 29.749 | −13.085 | 85.311 | 1.00 | 26.93 |
| ATOM | 2585 | O | GLU | 339 | 30.940 | −13.345 | 85.209 | 1.00 | 27.69 |
| ATOM | 2586 | N | SER | 340 | 29.264 | −12.320 | 86.285 | 1.00 | 27.55 |
| ATOM | 2587 | CA | SER | 340 | 30.140 | −11.763 | 87.318 | 1.00 | 28.61 |
| ATOM | 2588 | CB | SER | 340 | 29.741 | −10.323 | 87.667 | 1.00 | 29.40 |
| ATOM | 2589 | OG | SER | 340 | 29.800 | −9.485 | 86.528 | 1.00 | 35.97 |
| ATOM | 2590 | C | SER | 340 | 30.029 | −12.615 | 88.583 | 1.00 | 27.94 |
| ATOM | 2591 | O | SER | 340 | 30.811 | −12.448 | 89.526 | 1.00 | 24.04 |
| ATOM | 2592 | N | ASP | 341 | 29.042 | −13.511 | 88.600 | 1.00 | 28.02 |
| ATOM | 2593 | CA | ASP | 341 | 28.812 | −14.387 | 89.748 | 1.00 | 29.66 |
| ATOM | 2594 | CB | ASP | 341 | 27.808 | −15.490 | 89.393 | 1.00 | 30.94 |
| ATOM | 2595 | CG | ASP | 341 | 27.296 | −16.227 | 90.620 | 1.00 | 33.11 |
| ATOM | 2596 | OD1 | ASP | 341 | 26.289 | −15.778 | 91.217 | 1.00 | 28.78 |
| ATOM | 2597 | OD2 | ASP | 341 | 27.918 | −17.247 | 90.991 | 1.00 | 32.82 |
| ATOM | 2598 | C | ASP | 341 | 30.137 | −15.003 | 90.163 | 1.00 | 30.38 |
| ATOM | 2599 | O | ASP | 341 | 30.853 | −15.564 | 89.342 | 1.00 | 30.59 |
| ATOM | 2600 | N | THR | 342 | 30.466 | −14.886 | 91.443 | 1.00 | 33.59 |
| ATOM | 2601 | CA | THR | 342 | 31.729 | −15.405 | 91.953 | 1.00 | 37.01 |
| ATOM | 2602 | CB | THR | 342 | 32.013 | −14.836 | 93.350 | 1.00 | 38.81 |
| ATOM | 2603 | OG1 | THR | 342 | 31.012 | −15.304 | 94.265 | 1.00 | 43.90 |
| ATOM | 2604 | CG2 | THR | 342 | 31.972 | −13.316 | 93.317 | 1.00 | 35.79 |
| ATOM | 2605 | C | THR | 342 | 31.780 | −16.929 | 92.027 | 1.00 | 37.87 |
| ATOM | 2606 | O | THR | 342 | 32.853 | −17.514 | 92.191 | 1.00 | 39.64 |
| ATOM | 2607 | N | GLY | 343 | 30.625 | −17.568 | 91.894 | 1.00 | 36.81 |
| ATOM | 2608 | CA | GLY | 343 | 30.578 | −19.018 | 91.970 | 1.00 | 39.26 |
| ATOM | 2609 | C | GLY | 343 | 29.631 | −19.515 | 93.053 | 1.00 | 38.98 |
| ATOM | 2610 | O | GLY | 343 | 29.293 | −20.695 | 93.090 | 1.00 | 39.46 |
| ATOM | 2611 | N | ASP | 344 | 29.204 | −18.615 | 93.935 | 1.00 | 38.20 |
| ATOM | 2612 | CA | ASP | 344 | 28.287 | −18.980 | 95.005 | 1.00 | 39.74 |
| ATOM | 2613 | CB | ASP | 344 | 28.480 | −18.071 | 96.231 | 1.00 | 39.14 |
| ATOM | 2614 | CG | ASP | 344 | 28.267 | −16.595 | 95.928 | 1.00 | 41.19 |
| ATOM | 2615 | OD1 | ASP | 344 | 27.733 | −16.256 | 94.848 | 1.00 | 39.57 |
| ATOM | 2616 | OD2 | ASP | 344 | 28.627 | −15.767 | 96.794 | 1.00 | 42.27 |
| ATOM | 2617 | C | ASP | 344 | 26.842 | −18.926 | 94.516 | 1.00 | 40.25 |
| ATOM | 2618 | O | ASP | 344 | 25.904 | −19.235 | 95.257 | 1.00 | 39.36 |
| ATOM | 2619 | N | ARG | 345 | 26.680 | −18.525 | 93.259 | 1.00 | 38.45 |
| ATOM | 2620 | CA | ARG | 345 | 25.374 | −18.449 | 92.618 | 1.00 | 37.30 |
| ATOM | 2621 | CB | ARG | 345 | 24.738 | −19.847 | 92.587 | 1.00 | 37.49 |
| ATOM | 2622 | CG | ARG | 345 | 25.657 | −20.935 | 92.044 | 1.00 | 38.81 |
| ATOM | 2623 | CD | ARG | 345 | 24.976 | −22.301 | 92.046 | 1.00 | 40.19 |
| ATOM | 2624 | NE | ARG | 345 | 25.790 | −23.327 | 91.397 | 1.00 | 42.18 |
| ATOM | 2625 | CZ | ARG | 345 | 26.730 | −24.051 | 91.999 | 1.00 | 43.19 |
| ATOM | 2626 | NH1 | ARG | 345 | 26.990 | −23.880 | 93.288 | 1.00 | 43.31 |
| ATOM | 2627 | NH2 | ARG | 345 | 27.421 | −24.947 | 91.302 | 1.00 | 40.56 |
| ATOM | 2628 | C | ARG | 345 | 24.397 | −17.456 | 93.246 | 1.00 | 37.06 |
| ATOM | 2629 | O | ARG | 345 | 23.231 | −17.395 | 92.837 | 1.00 | 35.44 |
| ATOM | 2630 | N | LYS | 346 | 24.855 | −16.681 | 94.228 | 1.00 | 37.09 |
| ATOM | 2631 | CA | LYS | 346 | 23.977 | −15.704 | 94.876 | 1.00 | 39.61 |
| ATOM | 2632 | CB | LYS | 346 | 24.710 | −14.964 | 96.005 | 1.00 | 43.18 |
| ATOM | 2633 | CG | LYS | 346 | 25.084 | −15.826 | 97.214 | 1.00 | 47.92 |
| ATOM | 2634 | CD | LYS | 346 | 25.835 | −15.009 | 98.285 | 1.00 | 50.48 |
| ATOM | 2635 | CE | LYS | 346 | 26.274 | −15.887 | 99.466 | 1.00 | 53.20 |
| ATOM | 2636 | NZ | LYS | 346 | 27.039 | −15.136 | 100.520 | 1.00 | 54.15 |
| ATOM | 2637 | C | LYS | 346 | 23.467 | −14.690 | 93.858 | 1.00 | 39.25 |
| ATOM | 2638 | O | LYS | 346 | 22.271 | −14.400 | 93.795 | 1.00 | 38.51 |
| ATOM | 2639 | N | GLN | 347 | 24.384 | −14.158 | 93.055 | 1.00 | 40.01 |
| ATOM | 2640 | CA | GLN | 347 | 24.036 | −13.169 | 92.037 | 1.00 | 39.62 |
| ATOM | 2641 | CB | GLN | 347 | 25.301 | −12.725 | 91.290 | 1.00 | 44.30 |
| ATOM | 2642 | CG | GLN | 347 | 25.117 | −11.507 | 90.403 | 1.00 | 50.12 |
| ATOM | 2643 | CD | GLN | 347 | 24.996 | −10.214 | 91.196 | 1.00 | 54.40 |
| ATOM | 2644 | OE1 | GLN | 347 | 24.699 | −9.153 | 90.637 | 1.00 | 57.36 |
| ATOM | 2645 | NE2 | GLN | 347 | 25.234 | −10.295 | 92.501 | 1.00 | 55.02 |
| ATOM | 2646 | C | GLN | 347 | 23.015 | −13.735 | 91.046 | 1.00 | 36.71 |
| ATOM | 2647 | O | GLN | 347 | 22.012 | −13.087 | 90.732 | 1.00 | 35.38 |
| ATOM | 2648 | N | ILE | 348 | 23.264 | −14.949 | 90.563 | 1.00 | 33.61 |
| ATOM | 2649 | CA | ILE | 348 | 22.360 | −15.579 | 89.610 | 1.00 | 30.26 |
| ATOM | 2650 | CB | ILE | 348 | 22.946 | −16.906 | 89.103 | 1.00 | 31.09 |
| ATOM | 2651 | CG2 | ILE | 348 | 21.983 | −17.561 | 88.102 | 1.00 | 24.14 |
| ATOM | 2652 | CG1 | ILE | 348 | 24.315 | −16.641 | 88.467 | 1.00 | 24.89 |
| ATOM | 2653 | CD1 | ILE | 348 | 25.016 | −17.870 | 87.989 | 1.00 | 26.20 |
| ATOM | 2654 | C | ILE | 348 | 20.990 | −15.836 | 90.231 | 1.00 | 32.47 |
| ATOM | 2655 | O | ILE | 348 | 19.946 | −15.578 | 89.607 | 1.00 | 28.48 |
| ATOM | 2656 | N | TYR | 349 | 20.996 | −16.330 | 91.468 | 1.00 | 33.64 |
| ATOM | 2657 | CA | TYR | 349 | 19.757 | −16.622 | 92.173 | 1.00 | 33.94 |
| ATOM | 2658 | CB | TYR | 349 | 20.023 | −17.189 | 93.566 | 1.00 | 35.19 |
| ATOM | 2659 | CG | TYR | 349 | 18.728 | −17.513 | 94.273 | 1.00 | 35.54 |
| ATOM | 2660 | CD1 | TYR | 349 | 18.085 | −18.737 | 94.064 | 1.00 | 35.44 |
| ATOM | 2661 | CE1 | TYR | 349 | 16.847 | −19.009 | 94.647 | 1.00 | 35.96 |
| ATOM | 2662 | CD2 | TYR | 349 | 18.100 | −16.569 | 95.083 | 1.00 | 34.28 |
| ATOM | 2663 | CE2 | TYR | 349 | 16.860 | −16.833 | 95.665 | 1.00 | 34.50 |
| ATOM | 2664 | CZ | TYR | 349 | 16.242 | −18.053 | 95.441 | 1.00 | 34.82 |
| ATOM | 2665 | OH | TYR | 349 | 15.007 | −18.305 | 95.990 | 1.00 | 39.44 |
| ATOM | 2666 | C | TYR | 349 | 18.888 | −15.390 | 92.339 | 1.00 | 35.45 |
| ATOM | 2667 | O | TYR | 349 | 17.698 | −15.419 | 92.042 | 1.00 | 37.11 |
| ATOM | 2668 | N | ASN | 350 | 19.475 | −14.312 | 92.846 | 1.00 | 37.18 |
| ATOM | 2669 | CA | ASN | 350 | 18.722 | −13.082 | 93.049 | 1.00 | 38.47 |
| ATOM | 2670 | CB | ASN | 350 | 19.617 | −11.985 | 93.630 | 1.00 | 40.65 |
| ATOM | 2671 | CG | ASN | 350 | 20.014 | −12.263 | 95.065 | 1.00 | 45.75 |
| ATOM | 2672 | OD1 | ASN | 350 | 19.176 | −12.638 | 95.893 | 1.00 | 45.11 |
| ATOM | 2673 | ND2 | ASN | 350 | 21.298 | −12.075 | 95.373 | 1.00 | 46.81 |
| ATOM | 2674 | C | ASN | 350 | 18.085 | −12.585 | 91.768 | 1.00 | 37.56 |
| ATOM | 2675 | O | ASN | 350 | 16.924 | −12.186 | 91.769 | 1.00 | 40.92 |
| ATOM | 2676 | N | ILE | 351 | 18.839 | −12.601 | 90.673 | 1.00 | 37.62 |
| ATOM | 2677 | CA | ILE | 351 | 18.310 | −12.139 | 89.395 | 1.00 | 37.09 |
| ATOM | 2678 | CB | ILE | 351 | 19.401 | −12.130 | 88.308 | 1.00 | 38.11 |
| ATOM | 2679 | CG2 | ILE | 351 | 18.771 | −11.955 | 86.938 | 1.00 | 37.56 |
| ATOM | 2680 | CG1 | ILE | 351 | 20.400 | −11.004 | 88.588 | 1.00 | 38.11 |
| ATOM | 2681 | CD1 | ILE | 351 | 21.726 | −11.178 | 87.879 | 1.00 | 36.24 |
| ATOM | 2682 | C | ILE | 351 | 17.144 | −12.997 | 88.921 | 1.00 | 36.57 |
| ATOM | 2683 | O | ILE | 351 | 16.120 | −12.474 | 88.479 | 1.00 | 38.22 |
| ATOM | 2684 | N | LEU | 352 | 17.291 | −14.314 | 89.012 | 1.00 | 35.96 |
| ATOM | 2685 | CA | LEU | 352 | 16.219 | −15.206 | 88.577 | 1.00 | 36.28 |
| ATOM | 2686 | CB | LEU | 352 | 16.740 | −16.640 | 88.443 | 1.00 | 32.41 |
| ATOM | 2687 | CG | LEU | 352 | 17.845 | −16.828 | 87.395 | 1.00 | 30.66 |
| ATOM | 2688 | CD1 | LEU | 352 | 18.465 | −18.226 | 87.496 | 1.00 | 25.83 |
| ATOM | 2689 | CD2 | LEU | 352 | 17.262 | −16.597 | 86.025 | 1.00 | 27.66 |
| ATOM | 2690 | C | LEU | 352 | 15.039 | −15.156 | 89.547 | 1.00 | 37.27 |
| ATOM | 2691 | O | LEU | 352 | 13.896 | −15.356 | 89.145 | 1.00 | 38.32 |
| ATOM | 2692 | N | SER | 353 | 15.322 | −14.888 | 90.819 | 1.00 | 39.41 |
| ATOM | 2693 | CA | SER | 353 | 14.279 | −14.794 | 91.838 | 1.00 | 42.13 |
| ATOM | 2694 | CB | SER | 353 | 14.893 | −14.708 | 93.237 | 1.00 | 43.72 |
| ATOM | 2695 | OG | SER | 353 | 13.883 | −14.546 | 94.224 | 1.00 | 48.17 |
| ATOM | 2696 | C | SER | 353 | 13.431 | −13.557 | 91.590 | 1.00 | 43.61 |
| ATOM | 2697 | O | SER | 353 | 12.229 | −13.552 | 91.858 | 1.00 | 42.99 |
| ATOM | 2698 | N | THR | 354 | 14.066 | −12.506 | 91.081 | 1.00 | 44.80 |
| ATOM | 2699 | CA | THR | 354 | 13.363 | −11.267 | 90.785 | 1.00 | 46.06 |
| ATOM | 2700 | CB | THR | 354 | 14.356 | −10.122 | 90.497 | 1.00 | 47.48 |
| ATOM | 2701 | OG1 | THR | 354 | 15.100 | −9.820 | 91.687 | 1.00 | 47.39 |
| ATOM | 2702 | CG2 | THR | 354 | 13.615 | −8.877 | 90.034 | 1.00 | 47.87 |
| ATOM | 2703 | C | THR | 354 | 12.446 | −11.455 | 89.579 | 1.00 | 46.06 |
| ATOM | 2704 | O | THR | 354 | 11.443 | −10.757 | 89.436 | 1.00 | 47.23 |
| ATOM | 2705 | N | LEU | 355 | 12.788 | −12.406 | 88.717 | 1.00 | 46.03 |
| ATOM | 2706 | CA | LEU | 355 | 11.983 | −12.679 | 87.533 | 1.00 | 46.26 |
| ATOM | 2707 | CB | LEU | 355 | 12.875 | −13.157 | 86.390 | 1.00 | 46.43 |
| ATOM | 2708 | CG | LEU | 355 | 14.030 | −12.210 | 86.063 | 1.00 | 46.85 |
| ATOM | 2709 | CD1 | LEU | 355 | 14.861 | −12.813 | 84.950 | 1.00 | 47.00 |
| ATOM | 2710 | CD2 | LEU | 355 | 13.497 | −10.844 | 85.660 | 1.00 | 45.99 |
| ATOM | 2711 | C | LEU | 355 | 10.908 | −13.722 | 87.821 | 1.00 | 46.88 |
| ATOM | 2712 | O | LEU | 355 | 10.370 | −14.346 | 86.902 | 1.00 | 47.28 |
| ATOM | 2713 | N | GLY | 356 | 10.609 | −13.912 | 89.105 | 1.00 | 47.29 |
| ATOM | 2714 | CA | GLY | 356 | 9.586 | −14.858 | 89.511 | 1.00 | 44.74 |
| ATOM | 2715 | C | GLY | 356 | 9.959 | −16.321 | 89.396 | 1.00 | 44.45 |
| ATOM | 2716 | O | GLY | 356 | 9.097 | −17.163 | 89.146 | 1.00 | 45.09 |
| ATOM | 2717 | N | LEU | 357 | 11.235 | −16.635 | 89.575 | 1.00 | 43.26 |
| ATOM | 2718 | CA | LEU | 357 | 11.681 | −18.018 | 89.485 | 1.00 | 41.29 |
| ATOM | 2719 | CB | LEU | 357 | 12.653 | −18.187 | 88.310 | 1.00 | 42.15 |
| ATOM | 2720 | CG | LEU | 357 | 12.171 | −17.833 | 86.896 | 1.00 | 41.21 |
| ATOM | 2721 | CD1 | LEU | 357 | 13.366 | −17.781 | 85.972 | 1.00 | 39.61 |
| ATOM | 2722 | CD2 | LEU | 357 | 11.153 | −18.849 | 86.393 | 1.00 | 39.50 |
| ATOM | 2723 | C | LEU | 357 | 12.361 | −18.455 | 90.780 | 1.00 | 40.57 |
| ATOM | 2724 | O | LEU | 357 | 12.780 | −17.627 | 91.590 | 1.00 | 38.53 |
| ATOM | 2725 | N | ARG | 358 | 12.448 | −19.766 | 90.970 | 1.00 | 39.68 |
| ATOM | 2726 | CA | ARG | 358 | 13.092 | −20.355 | 92.139 | 1.00 | 40.04 |
| ATOM | 2727 | CB | ARG | 358 | 12.048 | −20.916 | 93.112 | 1.00 | 42.61 |
| ATOM | 2728 | CG | ARG | 358 | 11.172 | −19.845 | 93.760 | 1.00 | 46.08 |
| ATOM | 2729 | CD | ARG | 358 | 12.019 | −18.871 | 94.560 | 1.00 | 49.74 |
| ATOM | 2730 | NE | ARG | 358 | 11.355 | −17.588 | 94.772 | 1.00 | 55.41 |
| ATOM | 2731 | CZ | ARG | 358 | 10.588 | −17.293 | 95.816 | 1.00 | 58.08 |
| ATOM | 2732 | NH1 | ARG | 358 | 10.376 | −18.195 | 96.771 | 1.00 | 59.09 |
| ATOM | 2733 | NH2 | ARG | 358 | 10.035 | −16.087 | 95.906 | 1.00 | 58.98 |
| ATOM | 2734 | C | ARG | 358 | 13.954 | −21.471 | 91.576 | 1.00 | 38.39 |
| ATOM | 2735 | O | ARG | 358 | 13.569 | −22.641 | 91.586 | 1.00 | 37.47 |
| ATOM | 2736 | N | PRO | 359 | 15.140 | −21.109 | 91.065 | 1.00 | 36.51 |
| ATOM | 2737 | CD | PRO | 359 | 15.664 | −19.728 | 91.087 | 1.00 | 36.88 |
| ATOM | 2738 | CA | PRO | 359 | 16.123 | −22.006 | 90.461 | 1.00 | 34.17 |
| ATOM | 2739 | CB | PRO | 359 | 17.035 | −21.039 | 89.722 | 1.00 | 35.29 |
| ATOM | 2740 | CG | PRO | 359 | 17.135 | −19.925 | 90.703 | 1.00 | 34.03 |
| ATOM | 2741 | C | PRO | 359 | 16.915 | −22.872 | 91.416 | 1.00 | 33.10 |
| ATOM | 2742 | O | PRO | 359 | 17.140 | −22.520 | 92.566 | 1.00 | 31.20 |
| ATOM | 2743 | N | SER | 360 | 17.365 | −24.004 | 90.899 | 1.00 | 33.97 |
| ATOM | 2744 | CA | SER | 360 | 18.183 | −24.931 | 91.658 | 1.00 | 34.21 |
| ATOM | 2745 | CB | SER | 360 | 17.912 | −26.363 | 91.210 | 1.00 | 34.53 |
| ATOM | 2746 | OG | SER | 360 | 18.287 | −26.530 | 89.851 | 1.00 | 33.54 |
| ATOM | 2747 | C | SER | 360 | 19.618 | −24.568 | 91.307 | 1.00 | 34.99 |
| ATOM | 2748 | O | SER | 360 | 19.855 | −23.673 | 90.495 | 1.00 | 35.49 |
| ATOM | 2749 | N | THR | 361 | 20.564 | −25.267 | 91.920 | 1.00 | 34.70 |
| ATOM | 2750 | CA | THR | 361 | 21.977 | −25.048 | 91.673 | 1.00 | 36.89 |
| ATOM | 2751 | CB | THR | 361 | 22.838 | −26.003 | 92.535 | 1.00 | 36.99 |
| ATOM | 2752 | OG1 | THR | 361 | 22.828 | −25.558 | 93.898 | 1.00 | 38.93 |
| ATOM | 2753 | CG2 | THR | 361 | 24.260 | −26.041 | 92.033 | 1.00 | 38.24 |
| ATOM | 2754 | C | THR | 361 | 22.303 | −25.291 | 90.201 | 1.00 | 37.14 |
| ATOM | 2755 | O | THR | 361 | 23.142 | −24.606 | 89.616 | 1.00 | 37.81 |
| ATOM | 2756 | N | THR | 362 | 21.635 | −26.273 | 89.612 | 1.00 | 35.92 |
| ATOM | 2757 | CA | THR | 362 | 21.865 | −26.614 | 88.223 | 1.00 | 34.91 |
| ATOM | 2758 | CB | THR | 362 | 21.369 | −28.037 | 87.914 | 1.00 | 36.12 |
| ATOM | 2759 | OG1 | THR | 362 | 19.969 | −28.117 | 88.199 | 1.00 | 40.45 |
| ATOM | 2760 | CG2 | THR | 362 | 22.113 | −29.063 | 88.771 | 1.00 | 34.62 |
| ATOM | 2761 | C | THR | 362 | 21.181 | −25.626 | 87.292 | 1.00 | 33.53 |
| ATOM | 2762 | O | THR | 362 | 21.684 | −25.360 | 86.205 | 1.00 | 33.46 |
| ATOM | 2763 | N | ASP | 363 | 20.034 | −25.091 | 87.698 | 1.00 | 31.06 |
| ATOM | 2764 | CA | ASP | 363 | 19.355 | −24.115 | 86.860 | 1.00 | 32.46 |
| ATOM | 2765 | CB | ASP | 363 | 18.018 | −23.690 | 87.468 | 1.00 | 34.45 |
| ATOM | 2766 | CG | ASP | 363 | 16.964 | −24.783 | 87.409 | 1.00 | 37.91 |
| ATOM | 2767 | OD1 | ASP | 363 | 16.889 | −25.504 | 86.388 | 1.00 | 38.99 |
| ATOM | 2768 | OD2 | ASP | 363 | 16.194 | −24.907 | 88.385 | 1.00 | 38.23 |
| ATOM | 2769 | C | ASP | 363 | 20.254 | −22.878 | 86.718 | 1.00 | 32.88 |
| ATOM | 2770 | O | ASP | 363 | 20.419 | −22.331 | 85.629 | 1.00 | 30.65 |
| ATOM | 2771 | N | CYS | 364 | 20.833 | −22.451 | 87.836 | 1.00 | 33.86 |
| ATOM | 2772 | CA | CYS | 364 | 21.712 | −21.292 | 87.860 | 1.00 | 32.22 |
| ATOM | 2773 | CB | CYS | 364 | 22.186 | −21.015 | 89.289 | 1.00 | 31.35 |
| ATOM | 2774 | SG | CYS | 364 | 20.915 | −20.338 | 90.389 | 1.00 | 31.77 |
| ATOM | 2775 | C | CYS | 364 | 22.914 | −21.493 | 86.950 | 1.00 | 30.91 |
| ATOM | 2776 | O | CYS | 364 | 23.207 | −20.645 | 86.119 | 1.00 | 30.71 |
| ATOM | 2777 | N | ASP | 365 | 23.608 | −22.614 | 87.107 | 1.00 | 31.25 |
| ATOM | 2778 | CA | ASP | 365 | 24.774 | −22.894 | 86.280 | 1.00 | 32.00 |
| ATOM | 2779 | CB | ASP | 365 | 25.389 | −24.243 | 86.659 | 1.00 | 32.78 |
| ATOM | 2780 | CG | ASP | 365 | 26.037 | −24.211 | 88.023 | 1.00 | 35.48 |
| ATOM | 2781 | OD1 | ASP | 365 | 26.017 | −23.127 | 88.650 | 1.00 | 37.01 |
| ATOM | 2782 | OD2 | ASP | 365 | 26.564 | −25.251 | 88.466 | 1.00 | 35.21 |
| ATOM | 2783 | C | ASP | 365 | 24.405 | −22.886 | 84.810 | 1.00 | 30.41 |
| ATOM | 2784 | O | ASP | 365 | 25.166 | −22.407 | 83.966 | 1.00 | 31.73 |
| ATOM | 2785 | N | ILE | 366 | 23.225 | −23.408 | 84.514 | 1.00 | 29.17 |
| ATOM | 2786 | CA | ILE | 366 | 22.739 | −23.462 | 83.148 | 1.00 | 30.58 |
| ATOM | 2787 | CB | ILE | 366 | 21.456 | −24.318 | 83.058 | 1.00 | 30.61 |
| ATOM | 2788 | CG2 | ILE | 366 | 20.779 | −24.118 | 81.712 | 1.00 | 28.15 |
| ATOM | 2789 | CG1 | ILE | 366 | 21.808 | −25.797 | 83.261 | 1.00 | 33.09 |
| ATOM | 2790 | CD1 | ILE | 366 | 20.577 | −26.702 | 83.405 | 1.00 | 32.69 |
| ATOM | 2791 | C | ILE | 366 | 22.462 | −22.066 | 82.576 | 1.00 | 29.08 |
| ATOM | 2792 | O | ILE | 366 | 22.729 | −21.815 | 81.405 | 1.00 | 28.78 |
| ATOM | 2793 | N | VAL | 367 | 21.906 | −21.170 | 83.386 | 1.00 | 27.52 |
| ATOM | 2794 | CA | VAL | 367 | 21.632 | −19.817 | 82.910 | 1.00 | 27.71 |
| ATOM | 2795 | CB | VAL | 367 | 20.803 | −19.021 | 83.943 | 1.00 | 26.66 |
| ATOM | 2796 | CG1 | VAL | 367 | 20.812 | −17.531 | 83.609 | 1.00 | 24.57 |
| ATOM | 2797 | CG2 | VAL | 367 | 19.373 | −19.535 | 83.928 | 1.00 | 26.09 |
| ATOM | 2798 | C | VAL | 367 | 22.979 | −19.143 | 82.643 | 1.00 | 28.05 |
| ATOM | 2799 | O | VAL | 367 | 23.144 | −18.409 | 81.670 | 1.00 | 28.53 |
| ATOM | 2800 | N | ARG | 368 | 23.940 | −19.436 | 83.508 | 1.00 | 27.74 |
| ATOM | 2801 | CA | ARG | 368 | 25.300 | −18.927 | 83.386 | 1.00 | 30.76 |
| ATOM | 2802 | CB | ARG | 368 | 26.172 | −19.575 | 84.458 | 1.00 | 31.66 |
| ATOM | 2803 | CG | ARG | 368 | 27.023 | −18.648 | 85.269 | 1.00 | 38.26 |
| ATOM | 2804 | CD | ARG | 368 | 28.312 | −18.282 | 84.579 | 1.00 | 41.00 |
| ATOM | 2805 | NE | ARG | 368 | 29.272 | −17.763 | 85.547 | 1.00 | 43.72 |
| ATOM | 2806 | CZ | ARG | 368 | 30.397 | −17.135 | 85.226 | 1.00 | 46.75 |
| ATOM | 2807 | NH1 | ARG | 368 | 30.710 | −16.938 | 83.954 | 1.00 | 48.06 |
| ATOM | 2808 | NH2 | ARG | 368 | 31.212 | −16.708 | 86.179 | 1.00 | 47.96 |
| ATOM | 2809 | C | ARG | 368 | 25.841 | −19.317 | 82.003 | 1.00 | 30.63 |
| ATOM | 2810 | O | ARG | 368 | 26.343 | −18.469 | 81.256 | 1.00 | 27.84 |
| ATOM | 2811 | N | ARG | 369 | 25.735 | −20.606 | 81.677 | 1.00 | 27.70 |
| ATOM | 2812 | CA | ARG | 369 | 26.228 | −21.115 | 80.399 | 1.00 | 28.24 |
| ATOM | 2813 | CB | ARG | 369 | 26.077 | −22.645 | 80.327 | 1.00 | 26.69 |
| ATOM | 2814 | CG | ARG | 369 | 27.044 | −23.429 | 81.224 | 1.00 | 29.04 |
| ATOM | 2815 | CD | ARG | 369 | 28.506 | −23.228 | 80.815 | 1.00 | 31.91 |
| ATOM | 2816 | NE | ARG | 369 | 28.752 | −23.683 | 79.445 | 1.00 | 35.74 |
| ATOM | 2817 | CZ | ARG | 369 | 29.117 | −22.892 | 78.439 | 1.00 | 36.75 |
| ATOM | 2818 | NH1 | ARG | 369 | 29.291 | −21.590 | 78.638 | 1.00 | 36.65 |
| ATOM | 2819 | NH2 | ARG | 369 | 29.291 | −23.400 | 77.225 | 1.00 | 36.11 |
| ATOM | 2820 | C | ARG | 369 | 25.528 | −20.472 | 79.208 | 1.00 | 27.14 |
| ATOM | 2821 | O | ARG | 369 | 26.160 | −20.188 | 78.189 | 1.00 | 28.06 |
| ATOM | 2822 | N | ALA | 370 | 24.224 | −20.252 | 79.327 | 1.00 | 25.64 |
| ATOM | 2823 | CA | ALA | 370 | 23.480 | −19.634 | 78.238 | 1.00 | 25.08 |
| ATOM | 2824 | CB | ALA | 370 | 21.991 | −19.587 | 78.574 | 1.00 | 25.47 |
| ATOM | 2825 | C | ALA | 370 | 24.015 | −18.218 | 78.006 | 1.00 | 25.14 |
| ATOM | 2826 | O | ALA | 370 | 24.196 | −17.793 | 76.870 | 1.00 | 25.23 |
| ATOM | 2827 | N | CYS | 371 | 24.268 | −17.491 | 79.087 | 1.00 | 24.15 |
| ATOM | 2828 | CA | CYS | 371 | 24.785 | −16.135 | 78.965 | 1.00 | 25.09 |
| ATOM | 2829 | CB | CYS | 371 | 24.855 | −15.467 | 80.338 | 1.00 | 22.74 |
| ATOM | 2830 | SG | CYS | 371 | 23.239 | −15.076 | 81.033 | 1.00 | 25.40 |
| ATOM | 2831 | C | CYS | 371 | 26.161 | −16.127 | 78.300 | 1.00 | 24.93 |
| ATOM | 2832 | O | CYS | 371 | 26.392 | −15.358 | 77.367 | 1.00 | 25.49 |
| ATOM | 2833 | N | GLU | 372 | 27.062 | −16.991 | 78.765 | 1.00 | 24.70 |
| ATOM | 2834 | CA | GLU | 372 | 28.411 | −17.073 | 78.207 | 1.00 | 26.69 |
| ATOM | 2835 | CB | GLU | 372 | 29.247 | −18.105 | 78.975 | 1.00 | 27.07 |
| ATOM | 2836 | CG | GLU | 372 | 29.232 | −17.890 | 80.481 | 1.00 | 32.77 |
| ATOM | 2837 | CD | GLU | 372 | 30.016 | −18.945 | 81.243 | 1.00 | 33.87 |
| ATOM | 2838 | OE1 | GLU | 372 | 29.905 | −20.139 | 80.892 | 1.00 | 36.95 |
| ATOM | 2839 | OE2 | GLU | 372 | 30.733 | −18.583 | 82.200 | 1.00 | 35.18 |
| ATOM | 2840 | C | GLU | 372 | 28.418 | −17.420 | 76.718 | 1.00 | 27.23 |
| ATOM | 2841 | O | GLU | 372 | 29.259 | −16.922 | 75.966 | 1.00 | 29.09 |
| ATOM | 2842 | N | SER | 373 | 27.489 | −18.273 | 76.296 | 1.00 | 25.93 |
| ATOM | 2843 | CA | SER | 373 | 27.403 | −18.664 | 74.894 | 1.00 | 27.07 |
| ATOM | 2844 | CB | SER | 373 | 26.393 | −19.803 | 74.718 | 1.00 | 25.93 |
| ATOM | 2845 | OG | SER | 373 | 26.784 | −20.951 | 75.457 | 1.00 | 32.56 |
| ATOM | 2846 | C | SER | 373 | 26.988 | −17.471 | 74.034 | 1.00 | 25.31 |
| ATOM | 2847 | O | SER | 373 | 27.585 | −17.207 | 72.998 | 1.00 | 24.49 |
| ATOM | 2848 | N | VAL | 374 | 25.962 | −16.754 | 74.475 | 1.00 | 25.87 |
| ATOM | 2849 | CA | VAL | 374 | 25.473 | −15.596 | 73.743 | 1.00 | 25.12 |
| ATOM | 2850 | CB | VAL | 374 | 24.139 | −15.103 | 74.319 | 1.00 | 26.07 |
| ATOM | 2851 | CG1 | VAL | 374 | 23.754 | −13.766 | 73.682 | 1.00 | 29.29 |
| ATOM | 2852 | CG2 | VAL | 374 | 23.055 | −16.127 | 74.061 | 1.00 | 25.56 |
| ATOM | 2853 | C | VAL | 374 | 26.465 | −14.429 | 73.742 | 1.00 | 24.54 |
| ATOM | 2854 | O | VAL | 374 | 26.657 | −13.792 | 72.714 | 1.00 | 25.64 |
| ATOM | 2855 | N | SER | 375 | 27.094 | −14.144 | 74.878 | 1.00 | 21.70 |
| ATOM | 2856 | CA | SER | 375 | 28.029 | −13.034 | 74.922 | 1.00 | 23.89 |
| ATOM | 2857 | CB | SER | 375 | 28.298 | −12.585 | 76.365 | 1.00 | 23.28 |
| ATOM | 2858 | OG | SER | 375 | 28.986 | −13.565 | 77.120 | 1.00 | 29.71 |
| ATOM | 2859 | C | SER | 375 | 29.324 | −13.391 | 74.210 | 1.00 | 24.77 |
| ATOM | 2860 | O | SER | 375 | 29.873 | −12.560 | 73.490 | 1.00 | 23.61 |
| ATOM | 2861 | N | THR | 376 | 29.805 | −14.623 | 74.386 | 1.00 | 23.54 |
| ATOM | 2862 | CA | THR | 376 | 31.029 | −15.052 | 73.707 | 1.00 | 23.38 |
| ATOM | 2863 | CB | THR | 376 | 31.444 | −16.501 | 74.096 | 1.00 | 23.76 |
| ATOM | 2864 | OG1 | THR | 376 | 31.874 | −16.527 | 75.458 | 1.00 | 26.36 |
| ATOM | 2865 | CG2 | THR | 376 | 32.594 | −16.987 | 73.222 | 1.00 | 21.48 |
| ATOM | 2866 | C | THR | 376 | 30.859 | −14.996 | 72.189 | 1.00 | 22.33 |
| ATOM | 2867 | O | THR | 376 | 31.810 | −14.694 | 71.465 | 1.00 | 23.88 |
| ATOM | 2868 | N | ARG | 377 | 29.660 | −15.293 | 71.695 | 1.00 | 20.80 |
| ATOM | 2869 | CA | ARG | 377 | 29.452 | −15.239 | 70.253 | 1.00 | 21.46 |
| ATOM | 2870 | CB | ARG | 377 | 28.141 | −15.918 | 69.839 | 1.00 | 22.21 |
| ATOM | 2871 | CG | ARG | 377 | 27.958 | −15.875 | 68.312 | 1.00 | 25.01 |
| ATOM | 2872 | CD | ARG | 377 | 26.601 | −16.377 | 67.827 | 1.00 | 27.70 |
| ATOM | 2873 | NE | ARG | 377 | 25.491 | −15.558 | 68.302 | 1.00 | 25.17 |
| ATOM | 2874 | CZ | ARG | 377 | 24.255 | −15.637 | 67.825 | 1.00 | 26.42 |
| ATOM | 2875 | NH1 | ARG | 377 | 23.973 | −16.492 | 66.850 | 1.00 | 25.23 |
| ATOM | 2876 | NH2 | ARG | 377 | 23.294 | −14.877 | 68.339 | 1.00 | 26.96 |
| ATOM | 2877 | C | ARG | 377 | 29.439 | −13.773 | 69.787 | 1.00 | 21.55 |
| ATOM | 2878 | O | ARG | 377 | 29.856 | −13.462 | 68.670 | 1.00 | 20.80 |
| ATOM | 2879 | N | ALA | 378 | 28.951 | −12.879 | 70.639 | 1.00 | 19.46 |
| ATOM | 2880 | CA | ALA | 378 | 28.927 | −11.463 | 70.302 | 1.00 | 21.17 |
| ATOM | 2881 | CB | ALA | 378 | 28.239 | −10.653 | 71.412 | 1.00 | 20.68 |
| ATOM | 2882 | C | ALA | 378 | 30.374 | −11.015 | 70.151 | 1.00 | 20.18 |
| ATOM | 2883 | O | ALA | 378 | 30.747 | −10.420 | 69.145 | 1.00 | 20.36 |
| ATOM | 2884 | N | ALA | 379 | 31.191 | −11.326 | 71.153 | 1.00 | 19.41 |
| ATOM | 2885 | CA | ALA | 379 | 32.600 | −10.950 | 71.138 | 1.00 | 20.64 |
| ATOM | 2886 | CB | ALA | 379 | 33.296 | −11.515 | 72.371 | 1.00 | 20.04 |
| ATOM | 2887 | C | ALA | 379 | 33.332 | −11.405 | 69.869 | 1.00 | 22.79 |
| ATOM | 2888 | O | ALA | 379 | 34.054 | −10.620 | 69.234 | 1.00 | 21.82 |
| ATOM | 2889 | N | HIS | 380 | 33.139 | −12.666 | 69.489 | 1.00 | 22.45 |
| ATOM | 2890 | CA | HIS | 380 | 33.803 | −13.208 | 68.305 | 1.00 | 22.78 |
| ATOM | 2891 | CB | HIS | 380 | 33.726 | −14.745 | 68.314 | 1.00 | 22.80 |
| ATOM | 2892 | CG | HIS | 380 | 34.584 | −15.384 | 69.364 | 1.00 | 26.52 |
| ATOM | 2893 | CD2 | HIS | 380 | 35.557 | −14.870 | 70.152 | 1.00 | 27.81 |
| ATOM | 2894 | ND1 | HIS | 380 | 34.499 | −16.720 | 69.687 | 1.00 | 28.99 |
| ATOM | 2895 | CE1 | HIS | 380 | 35.383 | −17.002 | 70.627 | 1.00 | 28.15 |
| ATOM | 2896 | NE2 | HIS | 380 | 36.039 | −15.896 | 70.927 | 1.00 | 28.70 |
| ATOM | 2897 | C | HIS | 380 | 33.242 | −12.657 | 66.994 | 1.00 | 22.38 |
| ATOM | 2898 | O | HIS | 380 | 33.988 | −12.368 | 66.073 | 1.00 | 20.71 |
| ATOM | 2899 | N | MET | 381 | 31.926 | −12.524 | 66.915 | 1.00 | 23.83 |
| ATOM | 2900 | CA | MET | 381 | 31.285 | −12.018 | 65.713 | 1.00 | 26.66 |
| ATOM | 2901 | CB | MET | 381 | 29.760 | −12.086 | 65.899 | 1.00 | 29.06 |
| ATOM | 2902 | CG | MET | 381 | 28.926 | −12.031 | 64.622 | 1.00 | 34.34 |
| ATOM | 2903 | SD | MET | 381 | 29.456 | −13.157 | 63.312 | 1.00 | 33.69 |
| ATOM | 2904 | CE | MET | 381 | 28.228 | −14.472 | 63.429 | 1.00 | 34.64 |
| ATOM | 2905 | C | MET | 381 | 31.781 | −10.580 | 65.509 | 1.00 | 27.50 |
| ATOM | 2906 | O | MET | 381 | 32.153 | −10.188 | 64.406 | 1.00 | 26.70 |
| ATOM | 2907 | N | CYS | 382 | 31.830 | −9.813 | 66.595 | 1.00 | 26.32 |
| ATOM | 2908 | CA | CYS | 382 | 32.302 | −8.441 | 66.536 | 1.00 | 24.87 |
| ATOM | 2909 | CB | CYS | 382 | 32.102 | −7.769 | 67.896 | 1.00 | 26.05 |
| ATOM | 2910 | SG | CYS | 382 | 32.389 | −5.962 | 67.931 | 1.00 | 26.70 |
| ATOM | 2911 | C | CYS | 382 | 33.785 | −8.355 | 66.122 | 1.00 | 24.60 |
| ATOM | 2912 | O | CYS | 382 | 34.187 | −7.457 | 65.360 | 1.00 | 19.92 |
| ATOM | 2913 | N | SER | 383 | 34.590 | −9.288 | 66.623 | 1.00 | 22.62 |
| ATOM | 2914 | CA | SER | 383 | 36.017 | −9.302 | 66.327 | 1.00 | 22.35 |
| ATOM | 2915 | CB | SER | 383 | 36.716 | −10.439 | 67.096 | 1.00 | 23.03 |
| ATOM | 2916 | OG | SER | 383 | 36.361 | −11.712 | 66.571 | 1.00 | 24.25 |
| ATOM | 2917 | C | SER | 383 | 36.272 | −9.463 | 64.834 | 1.00 | 23.77 |
| ATOM | 2918 | O | SER | 383 | 37.202 | −8.875 | 64.288 | 1.00 | 24.79 |
| ATOM | 2919 | N | ALA | 384 | 35.448 | −10.269 | 64.173 | 1.00 | 24.03 |
| ATOM | 2920 | CA | ALA | 384 | 35.612 | −10.480 | 62.743 | 1.00 | 25.52 |
| ATOM | 2921 | CB | ALA | 384 | 34.649 | −11.552 | 62.256 | 1.00 | 22.05 |
| ATOM | 2922 | C | ALA | 384 | 35.369 | −9.182 | 61.980 | 1.00 | 25.61 |
| ATOM | 2923 | O | ALA | 384 | 35.990 | −8.942 | 60.947 | 1.00 | 25.37 |
| ATOM | 2924 | N | GLY | 385 | 34.450 | −8.360 | 62.490 | 1.00 | 25.67 |
| ATOM | 2925 | CA | GLY | 385 | 34.134 | −7.098 | 61.842 | 1.00 | 23.86 |
| ATOM | 2926 | C | GLY | 385 | 35.289 | −6.128 | 61.944 | 1.00 | 20.99 |
| ATOM | 2927 | O | GLY | 385 | 35.702 | −5.531 | 60.960 | 1.00 | 22.47 |
| ATOM | 2928 | N | LEU | 386 | 35.811 | −5.962 | 63.148 | 1.00 | 22.82 |
| ATOM | 2929 | CA | LEU | 386 | 36.937 | −5.065 | 63.364 | 1.00 | 25.33 |
| ATOM | 2930 | CB | LEU | 386 | 37.259 | −4.971 | 64.850 | 1.00 | 23.48 |
| ATOM | 2931 | CG | LEU | 386 | 37.800 | −3.658 | 65.425 | 1.00 | 27.75 |
| ATOM | 2932 | CD1 | LEU | 386 | 38.641 | −4.007 | 66.641 | 1.00 | 26.18 |
| ATOM | 2933 | CD2 | LEU | 386 | 38.621 | −2.865 | 64.428 | 1.00 | 25.52 |
| ATOM | 2934 | C | LEU | 386 | 38.172 | −5.584 | 62.616 | 1.00 | 26.01 |
| ATOM | 2935 | O | LEU | 386 | 38.953 | −4.794 | 62.067 | 1.00 | 26.60 |
| ATOM | 2936 | N | ALA | 387 | 38.356 | −6.904 | 62.601 | 1.00 | 23.95 |
| ATOM | 2937 | CA | ALA | 387 | 39.509 | −7.482 | 61.902 | 1.00 | 24.13 |
| ATOM | 2938 | CB | ALA | 387 | 39.585 | −8.989 | 62.135 | 1.00 | 20.59 |
| ATOM | 2939 | C | ALA | 387 | 39.405 | −7.181 | 60.411 | 1.00 | 24.07 |
| ATOM | 2940 | O | ALA | 387 | 40.419 | −6.990 | 59.730 | 1.00 | 22.59 |
| ATOM | 2941 | N | GLY | 388 | 38.175 | −7.141 | 59.904 | 1.00 | 24.30 |
| ATOM | 2942 | CA | GLY | 388 | 37.975 | −6.838 | 58.497 | 1.00 | 24.40 |
| ATOM | 2943 | C | GLY | 388 | 38.380 | −5.398 | 58.203 | 1.00 | 25.62 |
| ATOM | 2944 | O | GLY | 388 | 39.048 | −5.114 | 57.205 | 1.00 | 25.24 |
| ATOM | 2945 | N | VAL | 389 | 37.974 | −4.488 | 59.084 | 1.00 | 25.15 |
| ATOM | 2946 | CA | VAL | 389 | 38.294 | −3.072 | 58.950 | 1.00 | 23.08 |
| ATOM | 2947 | CB | VAL | 389 | 37.581 | −2.259 | 60.057 | 1.00 | 21.38 |
| ATOM | 2948 | CG1 | VAL | 389 | 38.083 | −0.820 | 60.076 | 1.00 | 21.90 |
| ATOM | 2949 | CG2 | VAL | 389 | 36.078 | −2.303 | 59.819 | 1.00 | 20.64 |
| ATOM | 2950 | C | VAL | 389 | 39.802 | −2.858 | 59.034 | 1.00 | 24.13 |
| ATOM | 2951 | O | VAL | 389 | 40.402 | −2.198 | 58.178 | 1.00 | 25.99 |
| ATOM | 2952 | N | ILE | 390 | 40.424 | −3.429 | 60.054 | 1.00 | 24.21 |
| ATOM | 2953 | CA | ILE | 390 | 41.866 | −3.289 | 60.209 | 1.00 | 25.31 |
| ATOM | 2954 | CB | ILE | 390 | 42.317 | −3.883 | 61.576 | 1.00 | 25.21 |
| ATOM | 2955 | CG2 | ILE | 390 | 43.831 | −3.962 | 61.661 | 1.00 | 27.92 |
| ATOM | 2956 | CG1 | ILE | 390 | 41.778 | −2.993 | 62.708 | 1.00 | 26.03 |
| ATOM | 2957 | CD1 | ILE | 390 | 42.091 | −3.476 | 64.094 | 1.00 | 27.41 |
| ATOM | 2958 | C | ILE | 390 | 42.668 | −3.899 | 59.040 | 1.00 | 26.27 |
| ATOM | 2959 | O | ILE | 390 | 43.622 | −3.287 | 58.563 | 1.00 | 25.08 |
| ATOM | 2960 | N | ASN | 391 | 42.286 | −5.082 | 58.561 | 1.00 | 27.72 |
| ATOM | 2961 | CA | ASN | 391 | 43.026 | −5.689 | 57.448 | 1.00 | 29.87 |
| ATOM | 2962 | CB | ASN | 391 | 42.649 | −7.162 | 57.250 | 1.00 | 27.74 |
| ATOM | 2963 | CG | ASN | 391 | 43.147 | −8.044 | 58.375 | 1.00 | 29.54 |
| ATOM | 2964 | OD1 | ASN | 391 | 44.216 | −7.804 | 58.939 | 1.00 | 28.68 |
| ATOM | 2965 | ND2 | ASN | 391 | 42.383 | −9.079 | 58.699 | 1.00 | 26.84 |
| ATOM | 2966 | C | ASN | 391 | 42.805 | −4.930 | 56.144 | 1.00 | 31.14 |
| ATOM | 2967 | O | ASN | 391 | 43.688 | −4.903 | 55.281 | 1.00 | 29.49 |
| ATOM | 2968 | N | ARG | 392 | 41.627 | −4.331 | 55.991 | 1.00 | 31.07 |
| ATOM | 2969 | CA | ARG | 392 | 41.358 | −3.553 | 54.795 | 1.00 | 33.43 |
| ATOM | 2970 | CB | ARG | 392 | 39.921 | −3.018 | 54.780 | 1.00 | 35.04 |
| ATOM | 2971 | CG | ARG | 392 | 39.597 | −2.307 | 53.483 | 1.00 | 35.84 |
| ATOM | 2972 | CD | ARG | 392 | 38.614 | −1.173 | 53.650 | 1.00 | 37.18 |
| ATOM | 2973 | NE | ARG | 392 | 38.804 | −0.186 | 52.589 | 1.00 | 35.89 |
| ATOM | 2974 | CZ | ARG | 392 | 38.518 | −0.390 | 51.309 | 1.00 | 36.67 |
| ATOM | 2975 | NH1 | ARG | 392 | 38.006 | −1.550 | 50.911 | 1.00 | 38.42 |
| ATOM | 2976 | NH2 | ARG | 392 | 38.788 | 0.553 | 50.417 | 1.00 | 37.33 |
| ATOM | 2977 | C | ARG | 392 | 42.335 | −2.377 | 54.831 | 1.00 | 33.73 |
| ATOM | 2978 | O | ARG | 392 | 43.028 | −2.107 | 53.858 | 1.00 | 34.52 |
| ATOM | 2979 | N | MET | 393 | 42.396 | −1.691 | 55.967 | 1.00 | 34.05 |
| ATOM | 2980 | CA | MET | 393 | 43.298 | −0.554 | 56.126 | 1.00 | 35.93 |
| ATOM | 2981 | CB | MET | 393 | 43.119 | 0.073 | 57.517 | 1.00 | 32.21 |
| ATOM | 2982 | CG | MET | 393 | 41.801 | 0.834 | 57.692 | 1.00 | 28.72 |
| ATOM | 2983 | SD | MET | 393 | 41.530 | 1.348 | 59.400 | 1.00 | 27.28 |
| ATOM | 2984 | CE | MET | 393 | 42.652 | 2.753 | 59.533 | 1.00 | 24.26 |
| ATOM | 2985 | C | MET | 393 | 44.751 | −0.979 | 55.947 | 1.00 | 39.48 |
| ATOM | 2986 | O | MET | 393 | 45.579 | −0.216 | 55.448 | 1.00 | 39.63 |
| ATOM | 2987 | N | ARG | 394 | 45.049 | −2.205 | 56.364 | 1.00 | 43.20 |
| ATOM | 2988 | CA | ARG | 394 | 46.391 | −2.766 | 56.277 | 1.00 | 45.79 |
| ATOM | 2989 | CB | ARG | 394 | 46.381 | −4.180 | 56.870 | 1.00 | 49.86 |
| ATOM | 2990 | CG | ARG | 394 | 47.670 | −4.595 | 57.551 | 1.00 | 53.76 |
| ATOM | 2991 | CD | ARG | 394 | 48.587 | −5.335 | 56.612 | 1.00 | 56.09 |
| ATOM | 2992 | NE | ARG | 394 | 49.896 | −5.554 | 57.217 | 1.00 | 60.36 |
| ATOM | 2993 | CZ | ARG | 394 | 50.797 | −4.596 | 57.411 | 1.00 | 60.35 |
| ATOM | 2994 | NH1 | ARG | 394 | 50.528 | −3.353 | 57.042 | 1.00 | 61.48 |
| ATOM | 2995 | NH2 | ARG | 394 | 51.964 | −4.878 | 57.978 | 1.00 | 60.51 |
| ATOM | 2996 | C | ARG | 394 | 46.912 | −2.792 | 54.835 | 1.00 | 46.90 |
| ATOM | 2997 | O | ARG | 394 | 48.117 | −2.697 | 54.606 | 1.00 | 44.95 |
| ATOM | 2998 | N | GLU | 395 | 46.005 | −2.906 | 53.869 | 1.00 | 48.68 |
| ATOM | 2999 | CA | GLU | 395 | 46.387 | −2.943 | 52.459 | 1.00 | 52.84 |
| ATOM | 3000 | CB | GLU | 395 | 45.165 | −3.275 | 51.590 | 1.00 | 54.51 |
| ATOM | 3001 | CG | GLU | 395 | 44.388 | −4.508 | 52.051 | 1.00 | 60.85 |
| ATOM | 3002 | CD | GLU | 395 | 43.310 | −4.952 | 51.061 | 1.00 | 64.84 |
| ATOM | 3003 | OE1 | GLU | 395 | 42.485 | −4.105 | 50.642 | 1.00 | 65.83 |
| ATOM | 3004 | OE2 | GLU | 395 | 43.286 | −6.155 | 50.708 | 1.00 | 66.43 |
| ATOM | 3005 | C | GLU | 395 | 47.008 | −1.621 | 51.991 | 1.00 | 54.64 |
| ATOM | 3006 | O | GLU | 395 | 47.791 | −1.594 | 51.039 | 1.00 | 53.71 |
| ATOM | 3007 | N | SER | 396 | 46.660 | −0.528 | 52.666 | 1.00 | 56.54 |
| ATOM | 3008 | CA | SER | 396 | 47.179 | 0.794 | 52.313 | 1.00 | 58.22 |
| ATOM | 3009 | CB | SER | 396 | 46.037 | 1.808 | 52.266 | 1.00 | 57.21 |
| ATOM | 3010 | OG | SER | 396 | 44.980 | 1.340 | 51.448 | 1.00 | 59.52 |
| ATOM | 3011 | C | SER | 396 | 48.221 | 1.268 | 53.318 | 1.00 | 60.22 |
| ATOM | 3012 | O | SER | 396 | 48.394 | 2.468 | 53.527 | 1.00 | 60.38 |
| ATOM | 3013 | N | ARG | 397 | 48.915 | 0.324 | 53.941 | 1.00 | 62.22 |
| ATOM | 3014 | CA | ARG | 397 | 49.924 | 0.663 | 54.933 | 1.00 | 64.67 |
| ATOM | 3015 | CB | ARG | 397 | 49.430 | 0.260 | 56.324 | 1.00 | 65.24 |
| ATOM | 3016 | CG | ARG | 397 | 49.798 | 1.218 | 57.444 | 1.00 | 67.16 |
| ATOM | 3017 | CD | ARG | 397 | 49.178 | 2.596 | 57.244 | 1.00 | 68.03 |
| ATOM | 3018 | NE | ARG | 397 | 48.803 | 3.208 | 58.516 | 1.00 | 69.13 |
| ATOM | 3019 | CZ | ARG | 397 | 47.681 | 2.933 | 59.178 | 1.00 | 70.58 |
| ATOM | 3020 | NH1 | ARG | 397 | 46.813 | 2.059 | 58.687 | 1.00 | 71.37 |
| ATOM | 3021 | NH2 | ARG | 397 | 47.429 | 3.521 | 60.340 | 1.00 | 70.29 |
| ATOM | 3022 | C | ARG | 397 | 51.222 | −0.063 | 54.611 | 1.00 | 65.54 |
| ATOM | 3023 | O | ARG | 397 | 51.416 | −1.215 | 54.998 | 1.00 | 66.75 |
| ATOM | 3024 | N | SER | 398 | 52.106 | 0.621 | 53.894 | 1.00 | 66.86 |
| ATOM | 3025 | CA | SER | 398 | 53.388 | 0.052 | 53.508 | 1.00 | 67.48 |
| ATOM | 3026 | CB | SER | 398 | 53.980 | 0.832 | 52.331 | 1.00 | 67.48 |
| ATOM | 3027 | OG | SER | 398 | 53.155 | 0.725 | 51.181 | 1.00 | 66.93 |
| ATOM | 3028 | C | SER | 398 | 54.358 | 0.063 | 54.679 | 1.00 | 68.36 |
| ATOM | 3029 | O | SER | 398 | 55.036 | 1.063 | 54.934 | 1.00 | 69.35 |
| ATOM | 3030 | N | GLU | 399 | 54.413 | −1.059 | 55.388 | 1.00 | 67.90 |
| ATOM | 3031 | CA | GLU | 399 | 55.297 | −1.206 | 56.533 | 1.00 | 68.16 |
| ATOM | 3032 | CB | GLU | 399 | 55.002 | −0.126 | 57.564 | 1.00 | 68.95 |
| ATOM | 3033 | CG | GLU | 399 | 53.540 | 0.020 | 57.889 | 1.00 | 71.05 |
| ATOM | 3034 | CD | GLU | 399 | 53.261 | 1.318 | 58.598 | 1.00 | 71.37 |
| ATOM | 3035 | OE1 | GLU | 399 | 53.871 | 1.545 | 59.662 | 1.00 | 72.25 |
| ATOM | 3036 | OE2 | GLU | 399 | 52.443 | 2.111 | 58.089 | 1.00 | 71.32 |
| ATOM | 3037 | C | GLU | 399 | 55.167 | −2.581 | 57.168 | 1.00 | 67.57 |
| ATOM | 3038 | O | GLU | 399 | 54.078 | −3.155 | 57.232 | 1.00 | 67.34 |
| ATOM | 3039 | N | ASP | 400 | 56.301 | −3.091 | 57.635 | 1.00 | 66.86 |
| ATOM | 3040 | CA | ASP | 400 | 56.397 | −4.400 | 58.265 | 1.00 | 65.75 |
| ATOM | 3041 | CB | ASP | 400 | 57.739 | −4.507 | 58.989 | 1.00 | 68.55 |
| ATOM | 3042 | CG | ASP | 400 | 58.892 | −3.961 | 58.157 | 1.00 | 71.49 |
| ATOM | 3043 | OD1 | ASP | 400 | 59.015 | −4.356 | 56.976 | 1.00 | 72.29 |
| ATOM | 3044 | OD2 | ASP | 400 | 59.675 | −3.136 | 58.682 | 1.00 | 72.38 |
| ATOM | 3045 | C | ASP | 400 | 55.247 | −4.676 | 59.233 | 1.00 | 63.41 |
| ATOM | 3046 | O | ASP | 400 | 54.385 | −5.514 | 58.962 | 1.00 | 63.27 |
| ATOM | 3047 | N | VAL | 401 | 55.241 | −3.973 | 60.361 | 1.00 | 59.50 |
| ATOM | 3048 | CA | VAL | 401 | 54.193 | −4.138 | 61.360 | 1.00 | 55.59 |
| ATOM | 3049 | CB | VAL | 401 | 54.789 | −4.439 | 62.757 | 1.00 | 55.81 |
| ATOM | 3050 | CG1 | VAL | 401 | 53.698 | −4.375 | 63.818 | 1.00 | 54.69 |
| ATOM | 3051 | CG2 | VAL | 401 | 55.442 | −5.817 | 62.757 | 1.00 | 54.18 |
| ATOM | 3052 | C | VAL | 401 | 53.345 | −2.876 | 61.454 | 1.00 | 53.78 |
| ATOM | 3053 | O | VAL | 401 | 53.841 | −1.807 | 61.820 | 1.00 | 53.39 |
| ATOM | 3054 | N | MET | 402 | 52.065 | −2.991 | 61.114 | 1.00 | 50.91 |
| ATOM | 3055 | CA | MET | 402 | 51.190 | −1.834 | 61.194 | 1.00 | 47.59 |
| ATOM | 3056 | CB | MET | 402 | 49.992 | −1.958 | 60.250 | 1.00 | 46.98 |
| ATOM | 3057 | CG | MET | 402 | 49.043 | −0.768 | 60.387 | 1.00 | 47.22 |
| ATOM | 3058 | SD | MET | 402 | 47.505 | −0.874 | 59.461 | 1.00 | 48.69 |
| ATOM | 3059 | CE | MET | 402 | 46.622 | −2.099 | 60.439 | 1.00 | 48.15 |
| ATOM | 3060 | C | MET | 402 | 50.670 | −1.643 | 62.605 | 1.00 | 44.98 |
| ATOM | 3061 | O | MET | 402 | 49.945 | −2.483 | 63.134 | 1.00 | 43.92 |
| ATOM | 3062 | N | ARG | 403 | 51.054 | −0.533 | 63.219 | 1.00 | 43.27 |
| ATOM | 3063 | CA | ARG | 403 | 50.587 | −0.229 | 64.556 | 1.00 | 41.71 |
| ATOM | 3064 | CB | ARG | 403 | 51.673 | 0.484 | 65.350 | 1.00 | 45.65 |
| ATOM | 3065 | CG | ARG | 403 | 52.903 | −0.356 | 65.596 | 1.00 | 52.20 |
| ATOM | 3066 | CD | ARG | 403 | 53.973 | 0.474 | 66.262 | 1.00 | 57.99 |
| ATOM | 3067 | NE | ARG | 403 | 55.137 | −0.324 | 66.630 | 1.00 | 65.47 |
| ATOM | 3068 | CZ | ARG | 403 | 56.251 | 0.184 | 67.149 | 1.00 | 68.76 |
| ATOM | 3069 | NH1 | ARG | 403 | 56.349 | 1.493 | 67.357 | 1.00 | 69.34 |
| ATOM | 3070 | NH2 | ARG | 403 | 57.265 | −0.615 | 67.468 | 1.00 | 69.59 |
| ATOM | 3071 | C | ARG | 403 | 49.388 | 0.685 | 64.372 | 1.00 | 37.99 |
| ATOM | 3072 | O | ARG | 403 | 49.471 | 1.692 | 63.679 | 1.00 | 37.13 |
| ATOM | 3073 | N | ILE | 404 | 48.267 | 0.322 | 64.975 | 1.00 | 34.39 |
| ATOM | 3074 | CA | ILE | 404 | 47.069 | 1.129 | 64.854 | 1.00 | 31.53 |
| ATOM | 3075 | CB | ILE | 404 | 46.161 | 0.577 | 63.735 | 1.00 | 33.38 |
| ATOM | 3076 | CG2 | ILE | 404 | 45.681 | −0.829 | 64.096 | 1.00 | 32.57 |
| ATOM | 3077 | CG1 | ILE | 404 | 44.987 | 1.524 | 63.500 | 1.00 | 35.77 |
| ATOM | 3078 | CD1 | ILE | 404 | 44.144 | 1.153 | 62.300 | 1.00 | 38.45 |
| ATOM | 3079 | C | ILE | 404 | 46.322 | 1.152 | 66.179 | 1.00 | 28.96 |
| ATOM | 3080 | O | ILE | 404 | 46.393 | 0.204 | 66.956 | 1.00 | 29.35 |
| ATOM | 3081 | N | THR | 405 | 45.632 | 2.250 | 66.453 | 1.00 | 28.84 |
| ATOM | 3082 | CA | THR | 405 | 44.874 | 2.359 | 67.693 | 1.00 | 27.84 |
| ATOM | 3083 | CB | THR | 405 | 45.323 | 3.558 | 68.535 | 1.00 | 26.65 |
| ATOM | 3084 | OG1 | THR | 405 | 46.663 | 3.335 | 68.990 | 1.00 | 30.48 |
| ATOM | 3085 | CG2 | THR | 405 | 44.428 | 3.715 | 69.749 | 1.00 | 27.32 |
| ATOM | 3086 | C | THR | 405 | 43.387 | 2.460 | 67.408 | 1.00 | 27.13 |
| ATOM | 3087 | O | THR | 405 | 42.964 | 3.127 | 66.462 | 1.00 | 24.36 |
| ATOM | 3088 | N | VAL | 406 | 42.604 | 1.786 | 68.245 | 1.00 | 25.61 |
| ATOM | 3089 | CA | VAL | 406 | 41.160 | 1.737 | 68.107 | 1.00 | 23.67 |
| ATOM | 3090 | CB | VAL | 406 | 40.705 | 0.244 | 67.973 | 1.00 | 23.64 |
| ATOM | 3091 | CG1 | VAL | 406 | 39.189 | 0.138 | 67.798 | 1.00 | 24.19 |
| ATOM | 3092 | CG2 | VAL | 406 | 41.405 | −0.399 | 66.783 | 1.00 | 21.36 |
| ATOM | 3093 | C | VAL | 406 | 40.493 | 2.392 | 69.320 | 1.00 | 26.21 |
| ATOM | 3094 | O | VAL | 406 | 40.763 | 2.018 | 70.469 | 1.00 | 26.86 |
| ATOM | 3095 | N | GLY | 407 | 39.644 | 3.389 | 69.072 | 1.00 | 25.61 |
| ATOM | 3096 | CA | GLY | 407 | 38.943 | 4.044 | 70.168 | 1.00 | 23.09 |
| ATOM | 3097 | C | GLY | 407 | 37.645 | 3.285 | 70.387 | 1.00 | 21.77 |
| ATOM | 3098 | O | GLY | 407 | 36.919 | 3.011 | 69.426 | 1.00 | 23.17 |
| ATOM | 3099 | N | VAL | 408 | 37.334 | 2.943 | 71.632 | 1.00 | 20.52 |
| ATOM | 3100 | CA | VAL | 408 | 36.128 | 2.167 | 71.907 | 1.00 | 21.51 |
| ATOM | 3101 | CB | VAL | 408 | 36.500 | 0.684 | 72.252 | 1.00 | 23.04 |
| ATOM | 3102 | CG1 | VAL | 408 | 35.237 | −0.176 | 72.351 | 1.00 | 19.52 |
| ATOM | 3103 | CG2 | VAL | 408 | 37.436 | 0.121 | 71.201 | 1.00 | 20.49 |
| ATOM | 3104 | C | VAL | 408 | 35.282 | 2.704 | 73.060 | 1.00 | 23.66 |
| ATOM | 3105 | O | VAL | 408 | 35.814 | 3.223 | 74.045 | 1.00 | 23.60 |
| ATOM | 3106 | N | ASP | 409 | 33.963 | 2.580 | 72.923 | 1.00 | 24.58 |
| ATOM | 3107 | CA | ASP | 409 | 33.040 | 2.992 | 73.975 | 1.00 | 26.70 |
| ATOM | 3108 | CB | ASP | 409 | 32.612 | 4.455 | 73.803 | 1.00 | 30.78 |
| ATOM | 3109 | CG | ASP | 409 | 31.909 | 4.998 | 75.041 | 1.00 | 31.51 |
| ATOM | 3110 | OD1 | ASP | 409 | 32.322 | 4.625 | 76.156 | 1.00 | 31.70 |
| ATOM | 3111 | OD2 | ASP | 409 | 30.955 | 5.794 | 74.910 | 1.00 | 35.70 |
| ATOM | 3112 | C | ASP | 409 | 31.824 | 2.083 | 73.898 | 1.00 | 25.68 |
| ATOM | 3113 | O | ASP | 409 | 31.639 | 1.396 | 72.901 | 1.00 | 27.99 |
| ATOM | 3114 | N | GLY | 410 | 30.999 | 2.079 | 74.943 | 1.00 | 28.67 |
| ATOM | 3115 | CA | GLY | 410 | 29.807 | 1.233 | 74.964 | 1.00 | 29.54 |
| ATOM | 3116 | C | GLY | 410 | 29.755 | 0.355 | 76.212 | 1.00 | 30.09 |
| ATOM | 3117 | O | GLY | 410 | 30.787 | −0.138 | 76.657 | 1.00 | 28.57 |
| ATOM | 3118 | N | SER | 411 | 28.560 | 0.150 | 76.767 | 1.00 | 30.89 |
| ATOM | 3119 | CA | SER | 411 | 28.392 | −0.649 | 77.983 | 1.00 | 32.71 |
| ATOM | 3120 | CB | SER | 411 | 26.941 | −0.554 | 78.490 | 1.00 | 32.88 |
| ATOM | 3121 | OG | SER | 411 | 26.011 | −0.884 | 77.473 | 1.00 | 36.82 |
| ATOM | 3122 | C | SER | 411 | 28.804 | −2.121 | 77.840 | 1.00 | 31.25 |
| ATOM | 3123 | O | SER | 411 | 29.480 | −2.661 | 78.712 | 1.00 | 29.96 |
| ATOM | 3124 | N | VAL | 412 | 28.398 | −2.768 | 76.754 | 1.00 | 29.78 |
| ATOM | 3125 | CA | VAL | 412 | 28.780 | −4.158 | 76.535 | 1.00 | 28.59 |
| ATOM | 3126 | CB | VAL | 412 | 28.264 | −4.665 | 75.174 | 1.00 | 29.68 |
| ATOM | 3127 | CG1 | VAL | 412 | 28.772 | −6.088 | 74.908 | 1.00 | 27.25 |
| ATOM | 3128 | CG2 | VAL | 412 | 26.739 | −4.642 | 75.173 | 1.00 | 29.93 |
| ATOM | 3129 | C | VAL | 412 | 30.307 | −4.320 | 76.584 | 1.00 | 29.24 |
| ATOM | 3130 | O | VAL | 412 | 30.831 | −5.145 | 77.340 | 1.00 | 28.78 |
| ATOM | 3131 | N | TYR | 413 | 31.023 | −3.522 | 75.796 | 1.00 | 27.57 |
| ATOM | 3132 | CA | TYR | 413 | 32.482 | −3.602 | 75.763 | 1.00 | 24.60 |
| ATOM | 3133 | CB | TYR | 413 | 33.049 | −2.730 | 74.645 | 1.00 | 19.87 |
| ATOM | 3134 | CG | TYR | 413 | 34.568 | −2.710 | 74.587 | 1.00 | 20.22 |
| ATOM | 3135 | CD1 | TYR | 413 | 35.270 | −3.566 | 73.735 | 1.00 | 21.52 |
| ATOM | 3136 | CE1 | TYR | 413 | 36.667 | −3.519 | 73.655 | 1.00 | 19.93 |
| ATOM | 3137 | CD2 | TYR | 413 | 35.300 | −1.819 | 75.363 | 1.00 | 14.63 |
| ATOM | 3138 | CE2 | TYR | 413 | 36.690 | −1.770 | 75.294 | 1.00 | 17.31 |
| ATOM | 3139 | CZ | TYR | 413 | 37.364 | −2.616 | 74.439 | 1.00 | 19.92 |
| ATOM | 3140 | OH | TYR | 413 | 38.737 | −2.547 | 74.362 | 1.00 | 23.08 |
| ATOM | 3141 | C | TYR | 413 | 33.151 | −3.193 | 77.072 | 1.00 | 26.48 |
| ATOM | 3142 | O | TYR | 413 | 34.085 | −3.849 | 77.534 | 1.00 | 26.86 |
| ATOM | 3143 | N | LYS | 414 | 32.690 | −2.108 | 77.669 | 1.00 | 26.13 |
| ATOM | 3144 | CA | LYS | 414 | 33.309 | −1.640 | 78.902 | 1.00 | 29.80 |
| ATOM | 3145 | CB | LYS | 414 | 33.001 | −0.147 | 79.117 | 1.00 | 29.42 |
| ATOM | 3146 | CG | LYS | 414 | 33.882 | 0.802 | 78.302 | 1.00 | 32.94 |
| ATOM | 3147 | CD | LYS | 414 | 33.558 | 2.275 | 78.559 | 1.00 | 34.12 |
| ATOM | 3148 | CE | LYS | 414 | 34.553 | 3.179 | 77.833 | 1.00 | 36.09 |
| ATOM | 3149 | NZ | LYS | 414 | 34.170 | 4.626 | 77.859 | 1.00 | 35.28 |
| ATOM | 3150 | C | LYS | 414 | 32.966 | −2.400 | 80.181 | 1.00 | 29.74 |
| ATOM | 3151 | O | LYS | 414 | 33.850 | −2.677 | 80.988 | 1.00 | 28.77 |
| ATOM | 3152 | N | LEU | 415 | 31.696 | −2.749 | 80.357 | 1.00 | 30.99 |
| ATOM | 3153 | CA | LEU | 415 | 31.255 | −3.395 | 81.591 | 1.00 | 34.39 |
| ATOM | 3154 | CB | LEU | 415 | 29.942 | −2.738 | 82.041 | 1.00 | 34.94 |
| ATOM | 3155 | CG | LEU | 415 | 29.964 | −1.195 | 82.012 | 1.00 | 38.17 |
| ATOM | 3156 | CD1 | LEU | 415 | 28.610 | −0.647 | 82.469 | 1.00 | 38.59 |
| ATOM | 3157 | CD2 | LEU | 415 | 31.080 | −0.660 | 82.901 | 1.00 | 34.42 |
| ATOM | 3158 | C | LEU | 415 | 31.113 | −4.923 | 81.657 | 1.00 | 34.46 |
| ATOM | 3159 | O | LEU | 415 | 31.202 | −5.493 | 82.741 | 1.00 | 33.89 |
| ATOM | 3160 | N | HIS | 416 | 30.886 | −5.586 | 80.531 | 1.00 | 34.56 |
| ATOM | 3161 | CA | HIS | 416 | 30.746 | −7.041 | 80.561 | 1.00 | 36.94 |
| ATOM | 3162 | CB | HIS | 416 | 30.394 | −7.572 | 79.175 | 1.00 | 39.81 |
| ATOM | 3163 | CG | HIS | 416 | 29.811 | −8.949 | 79.192 | 1.00 | 44.04 |
| ATOM | 3164 | CD2 | HIS | 416 | 28.536 | −9.375 | 79.038 | 1.00 | 43.02 |
| ATOM | 3165 | ND1 | HIS | 416 | 30.573 | −10.080 | 79.402 | 1.00 | 44.57 |
| ATOM | 3166 | CE1 | HIS | 416 | 29.791 | −11.144 | 79.374 | 1.00 | 44.76 |
| ATOM | 3167 | NE2 | HIS | 416 | 28.550 | −10.744 | 79.156 | 1.00 | 46.14 |
| ATOM | 3168 | C | HIS | 416 | 32.046 | −7.673 | 81.060 | 1.00 | 35.57 |
| ATOM | 3169 | O | HIS | 416 | 33.103 | −7.483 | 80.471 | 1.00 | 37.06 |
| ATOM | 3170 | N | PRO | 417 | 31.973 | −8.445 | 82.153 | 1.00 | 33.55 |
| ATOM | 3171 | CD | PRO | 417 | 30.727 | −8.999 | 82.700 | 1.00 | 32.04 |
| ATOM | 3172 | CA | PRO | 417 | 33.134 | −9.109 | 82.757 | 1.00 | 33.47 |
| ATOM | 3173 | CB | PRO | 417 | 32.504 | −10.219 | 83.614 | 1.00 | 31.67 |
| ATOM | 3174 | CG | PRO | 417 | 31.142 | −10.410 | 83.016 | 1.00 | 32.88 |
| ATOM | 3175 | C | PRO | 417 | 34.252 | −9.628 | 81.849 | 1.00 | 32.70 |
| ATOM | 3176 | O | PRO | 417 | 35.428 | −9.411 | 82.146 | 1.00 | 36.28 |
| ATOM | 3177 | N | SER | 418 | 33.929 | −10.302 | 80.752 | 1.00 | 29.70 |
| ATOM | 3178 | CA | SER | 418 | 35.015 | −10.808 | 79.915 | 1.00 | 28.37 |
| ATOM | 3179 | CB | SER | 418 | 35.215 | −12.314 | 80.163 | 1.00 | 30.65 |
| ATOM | 3180 | OG | SER | 418 | 35.798 | −12.555 | 81.439 | 1.00 | 35.61 |
| ATOM | 3181 | C | SER | 418 | 34.895 | −10.560 | 78.418 | 1.00 | 25.27 |
| ATOM | 3182 | O | SER | 418 | 35.730 | −11.028 | 77.648 | 1.00 | 23.69 |
| ATOM | 3183 | N | PHE | 419 | 33.856 | −9.846 | 78.004 | 1.00 | 21.86 |
| ATOM | 3184 | CA | PHE | 419 | 33.673 | −9.543 | 76.587 | 1.00 | 24.13 |
| ATOM | 3185 | CB | PHE | 419 | 32.551 | −8.522 | 76.407 | 1.00 | 22.03 |
| ATOM | 3186 | CG | PHE | 419 | 32.270 | −8.187 | 74.978 | 1.00 | 24.42 |
| ATOM | 3187 | CD1 | PHE | 419 | 31.273 | −8.860 | 74.276 | 1.00 | 23.32 |
| ATOM | 3188 | CD2 | PHE | 419 | 33.033 | −7.231 | 74.312 | 1.00 | 22.16 |
| ATOM | 3189 | CE1 | PHE | 419 | 31.038 | −8.593 | 72.932 | 1.00 | 23.49 |
| ATOM | 3190 | CE2 | PHE | 419 | 32.808 | −6.961 | 72.967 | 1.00 | 25.91 |
| ATOM | 3191 | CZ | PHE | 419 | 31.806 | −7.645 | 72.275 | 1.00 | 24.70 |
| ATOM | 3192 | C | PHE | 419 | 34.961 | −8.965 | 76.000 | 1.00 | 24.09 |
| ATOM | 3193 | O | PHE | 419 | 35.491 | −9.455 | 75.009 | 1.00 | 26.51 |
| ATOM | 3194 | N | LYS | 420 | 35.432 | −7.899 | 76.628 | 1.00 | 25.00 |
| ATOM | 3195 | CA | LYS | 420 | 36.641 | −7.179 | 76.238 | 1.00 | 26.79 |
| ATOM | 3196 | CB | LYS | 420 | 36.984 | −6.207 | 77.370 | 1.00 | 28.35 |
| ATOM | 3197 | CG | LYS | 420 | 38.241 | −5.396 | 77.229 | 1.00 | 30.04 |
| ATOM | 3198 | CD | LYS | 420 | 38.433 | −4.537 | 78.497 | 1.00 | 33.98 |
| ATOM | 3199 | CE | LYS | 420 | 37.170 | −3.740 | 78.832 | 1.00 | 31.09 |
| ATOM | 3200 | NZ | LYS | 420 | 37.322 | −2.923 | 80.067 | 1.00 | 36.69 |
| ATOM | 3201 | C | LYS | 420 | 37.819 | −8.118 | 75.968 | 1.00 | 25.76 |
| ATOM | 3202 | O | LYS | 420 | 38.446 | −8.064 | 74.911 | 1.00 | 25.94 |
| ATOM | 3203 | N | GLU | 421 | 38.111 | −8.961 | 76.951 | 1.00 | 24.13 |
| ATOM | 3204 | CA | GLU | 421 | 39.195 | −9.929 | 76.887 | 1.00 | 26.26 |
| ATOM | 3205 | CB | GLU | 421 | 39.204 | −10.781 | 78.155 | 1.00 | 32.38 |
| ATOM | 3206 | CG | GLU | 421 | 39.547 | −10.043 | 79.417 | 1.00 | 38.45 |
| ATOM | 3207 | CD | GLU | 421 | 38.700 | −8.798 | 79.664 | 1.00 | 41.54 |
| ATOM | 3208 | OE1 | GLU | 421 | 37.458 | −8.844 | 79.501 | 1.00 | 42.17 |
| ATOM | 3209 | OE2 | GLU | 421 | 39.300 | −7.767 | 80.053 | 1.00 | 42.62 |
| ATOM | 3210 | C | GLU | 421 | 39.075 | −10.864 | 75.699 | 1.00 | 24.57 |
| ATOM | 3211 | O | GLU | 421 | 40.017 | −11.023 | 74.930 | 1.00 | 25.86 |
| ATOM | 3212 | N | ARG | 422 | 37.921 | −11.509 | 75.576 | 1.00 | 24.00 |
| ATOM | 3213 | CA | ARG | 422 | 37.682 | −12.439 | 74.480 | 1.00 | 26.01 |
| ATOM | 3214 | CB | ARG | 422 | 36.284 | −13.063 | 74.610 | 1.00 | 27.36 |
| ATOM | 3215 | CG | ARG | 422 | 36.076 | −13.878 | 75.887 | 1.00 | 31.58 |
| ATOM | 3216 | CD | ARG | 422 | 34.600 | −14.053 | 76.188 | 1.00 | 35.39 |
| ATOM | 3217 | NE | ARG | 422 | 34.390 | −14.834 | 77.397 | 1.00 | 40.58 |
| ATOM | 3218 | CZ | ARG | 422 | 33.232 | −14.911 | 78.046 | 1.00 | 44.53 |
| ATOM | 3219 | NH1 | ARG | 422 | 32.171 | −14.243 | 77.596 | 1.00 | 41.79 |
| ATOM | 3220 | NH2 | ARG | 422 | 33.141 | −15.651 | 79.150 | 1.00 | 41.67 |
| ATOM | 3221 | C | ARG | 422 | 37.794 | −11.691 | 73.160 | 1.00 | 24.48 |
| ATOM | 3222 | O | ARG | 422 | 38.439 | −12.148 | 72.221 | 1.00 | 22.97 |
| ATOM | 3223 | N | PHE | 423 | 37.153 | −10.531 | 73.094 | 1.00 | 24.48 |
| ATOM | 3224 | CA | PHE | 423 | 37.189 | −9.737 | 71.879 | 1.00 | 22.97 |
| ATOM | 3225 | CB | PHE | 423 | 36.403 | −8.442 | 72.089 | 1.00 | 24.98 |
| ATOM | 3226 | CG | PHE | 423 | 36.494 | −7.484 | 70.939 | 1.00 | 25.21 |
| ATOM | 3227 | CD1 | PHE | 423 | 37.468 | −6.490 | 70.926 | 1.00 | 25.04 |
| ATOM | 3228 | CD2 | PHE | 423 | 35.618 | −7.584 | 69.861 | 1.00 | 23.47 |
| ATOM | 3229 | CE1 | PHE | 423 | 37.568 | −5.607 | 69.857 | 1.00 | 24.77 |
| ATOM | 3230 | CE2 | PHE | 423 | 35.710 | −6.708 | 68.784 | 1.00 | 25.48 |
| ATOM | 3231 | CZ | PHE | 423 | 36.684 | −5.715 | 68.780 | 1.00 | 24.31 |
| ATOM | 3232 | C | PHE | 423 | 38.629 | −9.442 | 71.456 | 1.00 | 21.03 |
| ATOM | 3233 | O | PHE | 423 | 38.989 | −9.680 | 70.308 | 1.00 | 19.38 |
| ATOM | 3234 | N | HIS | 424 | 39.454 | −8.952 | 72.381 | 1.00 | 20.46 |
| ATOM | 3235 | CA | HIS | 424 | 40.846 | −8.631 | 72.054 | 1.00 | 23.40 |
| ATOM | 3236 | CB | HIS | 424 | 41.602 | −8.128 | 73.293 | 1.00 | 24.89 |
| ATOM | 3237 | CG | HIS | 424 | 41.133 | −6.803 | 73.808 | 1.00 | 25.28 |
| ATOM | 3238 | CD2 | HIS | 424 | 40.391 | −5.828 | 73.230 | 1.00 | 24.67 |
| ATOM | 3239 | ND1 | HIS | 424 | 41.419 | −6.361 | 75.083 | 1.00 | 25.18 |
| ATOM | 3240 | CE1 | HIS | 424 | 40.869 | −5.174 | 75.269 | 1.00 | 22.64 |
| ATOM | 3241 | NE2 | HIS | 424 | 40.239 | −4.829 | 74.161 | 1.00 | 24.12 |
| ATOM | 3242 | C | HIS | 424 | 41.604 | −9.834 | 71.486 | 1.00 | 24.51 |
| ATOM | 3243 | O | HIS | 424 | 42.239 | −9.741 | 70.432 | 1.00 | 23.58 |
| ATOM | 3244 | N | ALA | 425 | 41.540 | −10.962 | 72.191 | 1.00 | 24.51 |
| ATOM | 3245 | CA | ALA | 425 | 42.242 | −12.164 | 71.746 | 1.00 | 26.94 |
| ATOM | 3246 | CB | ALA | 425 | 42.068 | −13.306 | 72.774 | 1.00 | 27.10 |
| ATOM | 3247 | C | ALA | 425 | 41.759 | −12.605 | 70.370 | 1.00 | 25.71 |
| ATOM | 3248 | O | ALA | 425 | 42.559 | −12.937 | 69.505 | 1.00 | 27.02 |
| ATOM | 3249 | N | SER | 426 | 40.453 | −12.600 | 70.151 | 1.00 | 24.30 |
| ATOM | 3250 | CA | SER | 426 | 39.967 | −13.003 | 68.850 | 1.00 | 23.93 |
| ATOM | 3251 | CB | SER | 426 | 38.450 | −13.142 | 68.863 | 1.00 | 20.85 |
| ATOM | 3252 | OG | SER | 426 | 38.007 | −13.582 | 67.596 | 1.00 | 21.86 |
| ATOM | 3253 | C | SER | 426 | 40.394 | −12.039 | 67.743 | 1.00 | 25.72 |
| ATOM | 3254 | O | SER | 426 | 40.760 | −12.483 | 66.660 | 1.00 | 25.40 |
| ATOM | 3255 | N | VAL | 427 | 40.363 | −10.727 | 68.007 | 1.00 | 27.03 |
| ATOM | 3256 | CA | VAL | 427 | 40.761 | −9.750 | 66.983 | 1.00 | 27.43 |
| ATOM | 3257 | CB | VAL | 427 | 40.591 | −8.269 | 67.450 | 1.00 | 28.91 |
| ATOM | 3258 | CG1 | VAL | 427 | 40.999 | −7.323 | 66.314 | 1.00 | 29.57 |
| ATOM | 3259 | CG2 | VAL | 427 | 39.150 | −7.990 | 67.852 | 1.00 | 27.73 |
| ATOM | 3260 | C | VAL | 427 | 42.226 | −9.919 | 66.601 | 1.00 | 28.67 |
| ATOM | 3261 | O | VAL | 427 | 42.582 | −9.858 | 65.424 | 1.00 | 27.30 |
| ATOM | 3262 | N | ARG | 428 | 43.076 | −10.119 | 67.603 | 1.00 | 28.43 |
| ATOM | 3263 | CA | ARG | 428 | 44.498 | −10.281 | 67.350 | 1.00 | 31.91 |
| ATOM | 3264 | CB | ARG | 428 | 45.273 | −10.231 | 68.670 | 1.00 | 31.80 |
| ATOM | 3265 | CG | ARG | 428 | 45.449 | −8.793 | 69.130 | 1.00 | 31.90 |
| ATOM | 3266 | CD | ARG | 428 | 45.662 | −8.639 | 70.617 | 1.00 | 34.40 |
| ATOM | 3267 | NE | ARG | 428 | 45.867 | −7.231 | 70.971 | 1.00 | 34.78 |
| ATOM | 3268 | CZ | ARG | 428 | 45.668 | −6.728 | 72.186 | 1.00 | 38.69 |
| ATOM | 3269 | NH1 | ARG | 428 | 45.251 | −7.516 | 73.172 | 1.00 | 38.56 |
| ATOM | 3270 | NH2 | ARG | 428 | 45.901 | −5.442 | 72.424 | 1.00 | 39.08 |
| ATOM | 3271 | C | ARG | 428 | 44.797 | −11.548 | 66.572 | 1.00 | 33.56 |
| ATOM | 3272 | O | ARG | 428 | 45.694 | −11.558 | 65.731 | 1.00 | 32.81 |
| ATOM | 3273 | N | ARG | 429 | 44.037 | −12.609 | 66.837 | 1.00 | 34.25 |
| ATOM | 3274 | CA | ARG | 429 | 44.224 | −13.859 | 66.115 | 1.00 | 33.42 |
| ATOM | 3275 | CB | ARG | 429 | 43.252 | −14.941 | 66.601 | 1.00 | 36.36 |
| ATOM | 3276 | CG | ARG | 429 | 43.756 | −15.769 | 67.760 | 1.00 | 43.73 |
| ATOM | 3277 | CD | ARG | 429 | 42.930 | −17.038 | 67.939 | 1.00 | 47.67 |
| ATOM | 3278 | NE | ARG | 429 | 41.561 | −16.789 | 68.398 | 1.00 | 51.58 |
| ATOM | 3279 | CZ | ARG | 429 | 41.222 | −16.467 | 69.646 | 1.00 | 51.70 |
| ATOM | 3280 | NH1 | ARG | 429 | 42.154 | −16.345 | 70.585 | 1.00 | 50.85 |
| ATOM | 3281 | NH2 | ARG | 429 | 39.945 | −16.288 | 69.962 | 1.00 | 49.95 |
| ATOM | 3282 | C | ARG | 429 | 43.960 | −13.618 | 64.639 | 1.00 | 32.81 |
| ATOM | 3283 | O | ARG | 429 | 44.610 | −14.215 | 63.783 | 1.00 | 32.29 |
| ATOM | 3284 | N | LEU | 430 | 43.001 | −12.741 | 64.345 | 1.00 | 30.18 |
| ATOM | 3285 | CA | LEU | 430 | 42.623 | −12.455 | 62.965 | 1.00 | 29.19 |
| ATOM | 3286 | CB | LEU | 430 | 41.132 | −12.109 | 62.904 | 1.00 | 29.15 |
| ATOM | 3287 | CG | LEU | 430 | 40.173 | −13.164 | 63.453 | 1.00 | 31.83 |
| ATOM | 3288 | CD1 | LEU | 430 | 38.746 | −12.629 | 63.437 | 1.00 | 28.32 |
| ATOM | 3289 | CD2 | LEU | 430 | 40.281 | −14.441 | 62.613 | 1.00 | 32.03 |
| ATOM | 3290 | C | LEU | 430 | 43.407 | −11.355 | 62.251 | 1.00 | 27.64 |
| ATOM | 3291 | O | LEU | 430 | 43.244 | −11.151 | 61.048 | 1.00 | 28.08 |
| ATOM | 3292 | N | THR | 431 | 44.261 | −10.645 | 62.966 | 1.00 | 28.04 |
| ATOM | 3293 | CA | THR | 431 | 44.988 | −9.567 | 62.326 | 1.00 | 31.15 |
| ATOM | 3294 | CB | THR | 431 | 44.569 | −8.201 | 62.934 | 1.00 | 30.03 |
| ATOM | 3295 | OG1 | THR | 431 | 44.666 | −8.254 | 64.363 | 1.00 | 31.84 |
| ATOM | 3296 | CG2 | THR | 431 | 43.137 | −7.879 | 62.561 | 1.00 | 26.93 |
| ATOM | 3297 | C | THR | 431 | 46.507 | −9.719 | 62.367 | 1.00 | 34.65 |
| ATOM | 3298 | O | THR | 431 | 47.190 | −9.015 | 63.101 | 1.00 | 34.08 |
| ATOM | 3299 | N | PRO | 432 | 47.049 | −10.655 | 61.566 | 1.00 | 37.36 |
| ATOM | 3300 | CD | PRO | 432 | 46.296 | −11.603 | 60.726 | 1.00 | 37.91 |
| ATOM | 3301 | CA | PRO | 432 | 48.489 | −10.923 | 61.484 | 1.00 | 38.59 |
| ATOM | 3302 | CB | PRO | 432 | 48.572 | −12.080 | 60.487 | 1.00 | 38.98 |
| ATOM | 3303 | CG | PRO | 432 | 47.245 | −12.758 | 60.630 | 1.00 | 40.10 |
| ATOM | 3304 | C | PRO | 432 | 49.224 | −9.689 | 60.969 | 1.00 | 39.65 |
| ATOM | 3305 | O | PRO | 432 | 48.712 | −8.968 | 60.113 | 1.00 | 39.80 |
| ATOM | 3306 | N | SER | 433 | 50.420 | −9.461 | 61.495 | 1.00 | 39.94 |
| ATOM | 3307 | CA | SER | 433 | 51.254 | −8.326 | 61.112 | 1.00 | 42.47 |
| ATOM | 3308 | CB | SER | 433 | 51.467 | −8.280 | 59.586 | 1.00 | 44.12 |
| ATOM | 3309 | OG | SER | 433 | 50.363 | −7.707 | 58.898 | 1.00 | 48.10 |
| ATOM | 3310 | C | SER | 433 | 50.687 | −6.996 | 61.598 | 1.00 | 42.26 |
| ATOM | 3311 | O | SER | 433 | 51.085 | −5.932 | 61.121 | 1.00 | 42.50 |
| ATOM | 3312 | N | CYS | 434 | 49.756 | −7.053 | 62.544 | 1.00 | 40.68 |
| ATOM | 3313 | CA | CYS | 434 | 49.184 | −5.831 | 63.092 | 1.00 | 40.64 |
| ATOM | 3314 | CB | CYS | 434 | 47.679 | −5.735 | 62.826 | 1.00 | 39.36 |
| ATOM | 3315 | SG | CYS | 434 | 47.196 | −5.674 | 61.111 | 1.00 | 39.36 |
| ATOM | 3316 | C | CYS | 434 | 49.398 | −5.789 | 64.590 | 1.00 | 40.17 |
| ATOM | 3317 | O | CYS | 434 | 49.258 | −6.801 | 65.281 | 1.00 | 40.51 |
| ATOM | 3318 | N | GLU | 435 | 49.743 | −4.609 | 65.081 | 1.00 | 38.91 |
| ATOM | 3319 | CA | GLU | 435 | 49.945 | −4.388 | 66.504 | 1.00 | 39.30 |
| ATOM | 3320 | CB | GLU | 435 | 51.302 | −3.733 | 66.738 | 1.00 | 42.29 |
| ATOM | 3321 | CG | GLU | 435 | 51.779 | −3.766 | 68.162 | 1.00 | 49.24 |
| ATOM | 3322 | CD | GLU | 435 | 53.072 | −2.993 | 68.340 | 1.00 | 53.77 |
| ATOM | 3323 | OE1 | GLU | 435 | 54.106 | −3.421 | 67.781 | 1.00 | 56.24 |
| ATOM | 3324 | OE2 | GLU | 435 | 53.047 | −1.950 | 69.032 | 1.00 | 54.88 |
| ATOM | 3325 | C | GLU | 435 | 48.801 | −3.430 | 66.839 | 1.00 | 37.06 |
| ATOM | 3326 | O | GLU | 435 | 48.866 | −2.241 | 66.532 | 1.00 | 34.30 |
| ATOM | 3327 | N | ILE | 436 | 47.749 | −3.971 | 67.449 | 1.00 | 36.39 |
| ATOM | 3328 | CA | ILE | 436 | 46.552 | −3.203 | 67.786 | 1.00 | 34.47 |
| ATOM | 3329 | CB | ILE | 436 | 45.280 | −4.040 | 67.508 | 1.00 | 34.15 |
| ATOM | 3330 | CG2 | ILE | 436 | 44.024 | −3.166 | 67.639 | 1.00 | 33.64 |
| ATOM | 3331 | CG1 | ILE | 436 | 45.357 | −4.633 | 66.100 | 1.00 | 36.06 |
| ATOM | 3332 | CD1 | ILE | 436 | 44.166 | −5.487 | 65.719 | 1.00 | 36.35 |
| ATOM | 3333 | C | ILE | 436 | 46.492 | −2.717 | 69.228 | 1.00 | 34.50 |
| ATOM | 3334 | O | ILE | 436 | 46.612 | −3.506 | 70.164 | 1.00 | 35.88 |
| ATOM | 3335 | N | THR | 437 | 46.308 | −1.411 | 69.405 | 1.00 | 32.66 |
| ATOM | 3336 | CA | THR | 437 | 46.196 | −0.837 | 70.741 | 1.00 | 30.32 |
| ATOM | 3337 | CB | THR | 437 | 47.134 | 0.370 | 70.930 | 1.00 | 29.83 |
| ATOM | 3338 | OG1 | THR | 437 | 48.496 | −0.060 | 70.833 | 1.00 | 33.74 |
| ATOM | 3339 | CG2 | THR | 437 | 46.925 | 0.996 | 72.294 | 1.00 | 28.96 |
| ATOM | 3340 | C | THR | 437 | 44.759 | −0.377 | 70.949 | 1.00 | 29.92 |
| ATOM | 3341 | O | THR | 437 | 44.177 | 0.293 | 70.090 | 1.00 | 28.24 |
| ATOM | 3342 | N | PHE | 438 | 44.179 | −0.750 | 72.083 | 1.00 | 29.43 |
| ATOM | 3343 | CA | PHE | 438 | 42.807 | −0.359 | 72.390 | 1.00 | 29.35 |
| ATOM | 3344 | CB | PHE | 438 | 41.991 | −1.567 | 72.853 | 1.00 | 27.92 |
| ATOM | 3345 | CG | PHE | 438 | 41.794 | −2.614 | 71.789 | 1.00 | 27.95 |
| ATOM | 3346 | CD1 | PHE | 438 | 42.695 | −3.661 | 71.648 | 1.00 | 27.90 |
| ATOM | 3347 | CD2 | PHE | 438 | 40.703 | −2.549 | 70.930 | 1.00 | 24.76 |
| ATOM | 3348 | CE1 | PHE | 438 | 42.505 | −4.634 | 70.662 | 1.00 | 29.21 |
| ATOM | 3349 | CE2 | PHE | 438 | 40.506 | −3.505 | 69.950 | 1.00 | 28.87 |
| ATOM | 3350 | CZ | PHE | 438 | 41.408 | −4.554 | 69.814 | 1.00 | 28.70 |
| ATOM | 3351 | C | PHE | 438 | 42.772 | 0.712 | 73.467 | 1.00 | 30.41 |
| ATOM | 3352 | O | PHE | 438 | 43.469 | 0.601 | 74.474 | 1.00 | 30.53 |
| ATOM | 3353 | N | ILE | 439 | 41.968 | 1.752 | 73.250 | 1.00 | 30.35 |
| ATOM | 3354 | CA | ILE | 439 | 41.839 | 2.832 | 74.220 | 1.00 | 31.89 |
| ATOM | 3355 | CB | ILE | 439 | 42.544 | 4.124 | 73.751 | 1.00 | 33.03 |
| ATOM | 3356 | CG2 | ILE | 439 | 42.233 | 5.269 | 74.721 | 1.00 | 36.00 |
| ATOM | 3357 | CG1 | ILE | 439 | 44.053 | 3.916 | 73.704 | 1.00 | 33.82 |
| ATOM | 3358 | CD1 | ILE | 439 | 44.818 | 5.165 | 73.296 | 1.00 | 36.93 |
| ATOM | 3359 | C | ILE | 439 | 40.373 | 3.158 | 74.420 | 1.00 | 32.85 |
| ATOM | 3360 | O | ILE | 439 | 39.603 | 3.157 | 73.467 | 1.00 | 33.09 |
| ATOM | 3361 | N | GLU | 440 | 39.991 | 3.442 | 75.659 | 1.00 | 35.09 |
| ATOM | 3362 | CA | GLU | 440 | 38.608 | 3.789 | 75.956 | 1.00 | 39.34 |
| ATOM | 3363 | CB | GLU | 440 | 38.133 | 3.041 | 77.199 | 1.00 | 37.95 |
| ATOM | 3364 | CG | GLU | 440 | 38.213 | 1.526 | 77.038 | 1.00 | 39.96 |
| ATOM | 3365 | CD | GLU | 440 | 37.837 | 0.773 | 78.298 | 1.00 | 40.82 |
| ATOM | 3366 | OE1 | GLU | 440 | 38.058 | −0.456 | 78.340 | 1.00 | 41.23 |
| ATOM | 3367 | OE2 | GLU | 440 | 37.318 | 1.403 | 79.245 | 1.00 | 41.08 |
| ATOM | 3368 | C | GLU | 440 | 38.495 | 5.298 | 76.156 | 1.00 | 41.28 |
| ATOM | 3369 | O | GLU | 440 | 39.356 | 5.918 | 76.769 | 1.00 | 42.10 |
| ATOM | 3370 | N | SER | 441 | 37.431 | 5.886 | 75.627 | 1.00 | 43.99 |
| ATOM | 3371 | CA | SER | 441 | 37.231 | 7.327 | 75.738 | 1.00 | 48.53 |
| ATOM | 3372 | CB | SER | 441 | 36.390 | 7.823 | 74.550 | 1.00 | 47.96 |
| ATOM | 3373 | OG | SER | 441 | 35.196 | 7.066 | 74.390 | 1.00 | 48.42 |
| ATOM | 3374 | C | SER | 441 | 36.577 | 7.752 | 77.051 | 1.00 | 50.20 |
| ATOM | 3375 | O | SER | 441 | 35.654 | 7.087 | 77.531 | 1.00 | 51.01 |
| ATOM | 3376 | N | GLU | 442 | 37.060 | 8.852 | 77.634 | 1.00 | 53.24 |
| ATOM | 3377 | CA | GLU | 442 | 36.490 | 9.359 | 78.885 | 1.00 | 55.51 |
| ATOM | 3378 | CB | GLU | 442 | 37.362 | 10.454 | 79.507 | 1.00 | 60.16 |
| ATOM | 3379 | CG | GLU | 442 | 36.822 | 10.936 | 80.859 | 1.00 | 65.44 |
| ATOM | 3380 | CD | GLU | 442 | 37.596 | 12.107 | 81.450 | 1.00 | 69.63 |
| ATOM | 3381 | OE1 | GLU | 442 | 38.824 | 11.984 | 81.667 | 1.00 | 71.27 |
| ATOM | 3382 | OE2 | GLU | 442 | 36.965 | 13.155 | 81.709 | 1.00 | 72.91 |
| ATOM | 3383 | C | GLU | 442 | 35.118 | 9.938 | 78.579 | 1.00 | 54.68 |
| ATOM | 3384 | O | GLU | 442 | 34.104 | 9.495 | 79.126 | 1.00 | 56.30 |
| ATOM | 3385 | N | GLU | 443 | 35.094 | 10.942 | 77.714 | 1.00 | 51.73 |
| ATOM | 3386 | CA | GLU | 443 | 33.840 | 11.555 | 77.307 | 1.00 | 51.12 |
| ATOM | 3387 | CB | GLU | 443 | 33.706 | 12.960 | 77.888 | 1.00 | 51.77 |
| ATOM | 3388 | CG | GLU | 443 | 32.561 | 13.086 | 78.869 | 1.00 | 49.05 |
| ATOM | 3389 | CD | GLU | 443 | 31.202 | 12.812 | 78.239 | 1.00 | 48.41 |
| ATOM | 3390 | OE1 | GLU | 443 | 30.245 | 12.572 | 79.006 | 1.00 | 48.06 |
| ATOM | 3391 | OE2 | GLU | 443 | 31.084 | 12.842 | 76.990 | 1.00 | 44.46 |
| ATOM | 3392 | C | GLU | 443 | 33.851 | 11.614 | 75.793 | 1.00 | 50.48 |
| ATOM | 3393 | O | GLU | 443 | 33.624 | 12.662 | 75.191 | 1.00 | 50.61 |
| ATOM | 3394 | N | GLY | 444 | 34.131 | 10.458 | 75.199 | 1.00 | 49.69 |
| ATOM | 3395 | CA | GLY | 444 | 34.213 | 10.321 | 73.760 | 1.00 | 46.29 |
| ATOM | 3396 | C | GLY | 444 | 33.300 | 11.190 | 72.928 | 1.00 | 45.39 |
| ATOM | 3397 | O | GLY | 444 | 33.786 | 12.031 | 72.181 | 1.00 | 44.10 |
| ATOM | 3398 | N | SER | 445 | 31.990 | 10.996 | 73.052 | 1.00 | 44.40 |
| ATOM | 3399 | CA | SER | 445 | 31.035 | 11.765 | 72.263 | 1.00 | 45.82 |
| ATOM | 3400 | CB | SER | 445 | 29.614 | 11.258 | 72.505 | 1.00 | 43.70 |
| ATOM | 3401 | OG | SER | 445 | 29.248 | 11.396 | 73.860 | 1.00 | 51.13 |
| ATOM | 3402 | C | SER | 445 | 31.108 | 13.265 | 72.523 | 1.00 | 45.79 |
| ATOM | 3403 | O | SER | 445 | 31.381 | 14.043 | 71.607 | 1.00 | 46.62 |
| ATOM | 3404 | N | GLY | 446 | 30.867 | 13.666 | 73.766 | 1.00 | 45.46 |
| ATOM | 3405 | CA | GLY | 446 | 30.924 | 15.075 | 74.112 | 1.00 | 44.61 |
| ATOM | 3406 | C | GLY | 446 | 32.176 | 15.778 | 73.615 | 1.00 | 44.65 |
| ATOM | 3407 | O | GLY | 446 | 32.085 | 16.754 | 72.872 | 1.00 | 45.17 |
| ATOM | 3408 | N | ARG | 447 | 33.344 | 15.286 | 74.024 | 1.00 | 44.10 |
| ATOM | 3409 | CA | ARG | 447 | 34.615 | 15.878 | 73.615 | 1.00 | 44.23 |
| ATOM | 3410 | CB | ARG | 447 | 35.765 | 15.244 | 74.396 | 1.00 | 44.71 |
| ATOM | 3411 | CG | ARG | 447 | 36.079 | 15.917 | 75.720 | 1.00 | 46.63 |
| ATOM | 3412 | CD | ARG | 447 | 36.405 | 14.896 | 76.794 | 1.00 | 48.87 |
| ATOM | 3413 | NE | ARG | 447 | 37.226 | 13.804 | 76.286 | 1.00 | 53.97 |
| ATOM | 3414 | CZ | ARG | 447 | 38.507 | 13.915 | 75.956 | 1.00 | 55.65 |
| ATOM | 3415 | NH1 | ARG | 447 | 39.130 | 15.076 | 76.085 | 1.00 | 56.71 |
| ATOM | 3416 | NH2 | ARG | 447 | 39.161 | 12.862 | 75.486 | 1.00 | 58.00 |
| ATOM | 3417 | C | ARG | 447 | 34.891 | 15.739 | 72.122 | 1.00 | 45.53 |
| ATOM | 3418 | O | ARG | 447 | 35.506 | 16.617 | 71.508 | 1.00 | 45.95 |
| ATOM | 3419 | N | GLY | 448 | 34.444 | 14.630 | 71.543 | 1.00 | 45.12 |
| ATOM | 3420 | CA | GLY | 448 | 34.667 | 14.395 | 70.129 | 1.00 | 43.75 |
| ATOM | 3421 | C | GLY | 448 | 33.915 | 15.390 | 69.275 | 1.00 | 44.42 |
| ATOM | 3422 | O | GLY | 448 | 34.497 | 16.033 | 68.401 | 1.00 | 43.25 |
| ATOM | 3423 | N | ALA | 449 | 32.617 | 15.508 | 69.530 | 1.00 | 44.33 |
| ATOM | 3424 | CA | ALA | 449 | 31.764 | 16.435 | 68.798 | 1.00 | 46.02 |
| ATOM | 3425 | CB | ALA | 449 | 30.349 | 16.393 | 69.362 | 1.00 | 44.02 |
| ATOM | 3426 | C | ALA | 449 | 32.334 | 17.852 | 68.901 | 1.00 | 47.27 |
| ATOM | 3427 | O | ALA | 449 | 32.388 | 18.585 | 67.910 | 1.00 | 46.99 |
| ATOM | 3428 | N | ALA | 450 | 32.771 | 18.226 | 70.100 | 1.00 | 47.32 |
| ATOM | 3429 | CA | ALA | 450 | 33.337 | 19.549 | 70.320 | 1.00 | 48.93 |
| ATOM | 3430 | CB | ALA | 450 | 33.590 | 19.771 | 71.803 | 1.00 | 48.70 |
| ATOM | 3431 | C | ALA | 450 | 34.630 | 19.752 | 69.537 | 1.00 | 49.10 |
| ATOM | 3432 | O | ALA | 450 | 34.795 | 20.770 | 68.864 | 1.00 | 51.55 |
| ATOM | 3433 | N | LEU | 451 | 35.546 | 18.792 | 69.625 | 1.00 | 47.13 |
| ATOM | 3434 | CA | LEU | 451 | 36.828 | 18.889 | 68.923 | 1.00 | 46.08 |
| ATOM | 3435 | CB | LEU | 451 | 37.693 | 17.661 | 69.226 | 1.00 | 43.72 |
| ATOM | 3436 | CG | LEU | 451 | 38.376 | 17.636 | 70.598 | 1.00 | 44.07 |
| ATOM | 3437 | CD1 | LEU | 451 | 38.798 | 16.218 | 70.955 | 1.00 | 41.74 |
| ATOM | 3438 | CD2 | LEU | 451 | 39.577 | 18.574 | 70.574 | 1.00 | 40.23 |
| ATOM | 3439 | C | LEU | 451 | 36.672 | 19.055 | 67.410 | 1.00 | 45.46 |
| ATOM | 3440 | O | LEU | 451 | 37.495 | 19.708 | 66.760 | 1.00 | 46.36 |
| ATOM | 3441 | N | VAL | 452 | 35.618 | 18.465 | 66.857 | 1.00 | 43.95 |
| ATOM | 3442 | CA | VAL | 452 | 35.348 | 18.552 | 65.428 | 1.00 | 44.38 |
| ATOM | 3443 | CB | VAL | 452 | 34.426 | 17.376 | 64.959 | 1.00 | 43.85 |
| ATOM | 3444 | CG1 | VAL | 452 | 33.998 | 17.576 | 63.513 | 1.00 | 41.59 |
| ATOM | 3445 | CG2 | VAL | 452 | 35.169 | 16.040 | 65.087 | 1.00 | 40.86 |
| ATOM | 3446 | C | VAL | 452 | 34.687 | 19.905 | 65.125 | 1.00 | 45.31 |
| ATOM | 3447 | O | VAL | 452 | 34.881 | 20.482 | 64.056 | 1.00 | 42.97 |
| ATOM | 3448 | N | SER | 453 | 33.912 | 20.411 | 66.077 | 1.00 | 46.60 |
| ATOM | 3449 | CA | SER | 453 | 33.253 | 21.693 | 65.900 | 1.00 | 49.07 |
| ATOM | 3450 | CB | SER | 453 | 32.204 | 21.902 | 66.986 | 1.00 | 47.21 |
| ATOM | 3451 | OG | SER | 453 | 31.146 | 20.972 | 66.845 | 1.00 | 44.37 |
| ATOM | 3452 | C | SER | 453 | 34.293 | 22.806 | 65.951 | 1.00 | 51.53 |
| ATOM | 3453 | O | SER | 453 | 34.150 | 23.820 | 65.281 | 1.00 | 52.56 |
| ATOM | 3454 | N | ALA | 454 | 35.352 | 22.593 | 66.728 | 1.00 | 54.40 |
| ATOM | 3455 | CA | ALA | 454 | 36.430 | 23.567 | 66.881 | 1.00 | 56.39 |
| ATOM | 3456 | CB | ALA | 454 | 37.336 | 23.158 | 68.031 | 1.00 | 55.74 |
| ATOM | 3457 | C | ALA | 454 | 37.259 | 23.751 | 65.614 | 1.00 | 58.75 |
| ATOM | 3458 | O | ALA | 454 | 37.863 | 24.807 | 65.408 | 1.00 | 59.45 |
| ATOM | 3459 | N | VAL | 455 | 37.310 | 22.719 | 64.779 | 1.00 | 60.29 |
| ATOM | 3460 | CA | VAL | 455 | 38.063 | 22.796 | 63.535 | 1.00 | 61.78 |
| ATOM | 3461 | CB | VAL | 455 | 38.603 | 21.416 | 63.112 | 1.00 | 61.44 |
| ATOM | 3462 | CG1 | VAL | 455 | 39.090 | 21.464 | 61.672 | 1.00 | 60.81 |
| ATOM | 3463 | CG2 | VAL | 455 | 39.737 | 21.005 | 64.031 | 1.00 | 60.68 |
| ATOM | 3464 | C | VAL | 455 | 37.152 | 23.330 | 62.442 | 1.00 | 63.56 |
| ATOM | 3465 | O | VAL | 455 | 37.550 | 24.176 | 61.643 | 1.00 | 63.25 |
| ATOM | 3466 | N | ALA | 456 | 35.921 | 22.835 | 62.416 | 1.00 | 65.38 |
| ATOM | 3467 | CA | ALA | 456 | 34.959 | 23.275 | 61.422 | 1.00 | 69.39 |
| ATOM | 3468 | CB | ALA | 456 | 33.751 | 22.354 | 61.423 | 1.00 | 68.17 |
| ATOM | 3469 | C | ALA | 456 | 34.522 | 24.709 | 61.710 | 1.00 | 73.10 |
| ATOM | 3470 | O | ALA | 456 | 33.975 | 25.382 | 60.837 | 1.00 | 73.04 |
| ATOM | 3471 | N | CYS | 457 | 34.771 | 25.170 | 62.935 | 1.00 | 77.06 |
| ATOM | 3472 | CA | CYS | 457 | 34.390 | 26.521 | 63.341 | 1.00 | 81.01 |
| ATOM | 3473 | CB | CYS | 457 | 34.192 | 26.599 | 64.856 | 1.00 | 80.51 |
| ATOM | 3474 | SG | CYS | 457 | 33.478 | 28.151 | 65.432 | 1.00 | 81.75 |
| ATOM | 3475 | C | CYS | 457 | 35.420 | 27.554 | 62.916 | 1.00 | 83.65 |
| ATOM | 3476 | O | CYS | 457 | 35.312 | 28.726 | 63.275 | 1.00 | 85.11 |
| ATOM | 3477 | N | LYS | 458 | 36.430 | 27.118 | 62.172 | 1.00 | 86.29 |
| ATOM | 3478 | CA | LYS | 458 | 37.441 | 28.041 | 61.683 | 1.00 | 89.14 |
| ATOM | 3479 | CB | LYS | 458 | 38.843 | 27.441 | 61.803 | 1.00 | 88.60 |
| ATOM | 3480 | CG | LYS | 458 | 39.932 | 28.486 | 61.632 | 1.00 | 89.51 |
| ATOM | 3481 | CD | LYS | 458 | 41.276 | 27.992 | 62.130 | 1.00 | 89.70 |
| ATOM | 3482 | CE | LYS | 458 | 42.257 | 29.146 | 62.269 | 1.00 | 89.22 |
| ATOM | 3483 | NZ | LYS | 458 | 41.718 | 30.194 | 63.180 | 1.00 | 88.81 |
| ATOM | 3484 | C | LYS | 458 | 37.096 | 28.310 | 60.232 | 1.00 | 91.26 |
| ATOM | 3485 | O | LYS | 458 | 37.936 | 28.733 | 59.438 | 1.00 | 91.56 |
| ATOM | 3486 | N | LYS | 459 | 35.834 | 28.043 | 59.901 | 1.00 | 93.94 |
| ATOM | 3487 | CA | LYS | 459 | 35.302 | 28.240 | 58.548 | 1.00 | 96.28 |
| ATOM | 3488 | CB | LYS | 459 | 35.323 | 26.923 | 57.765 | 1.00 | 96.25 |
| ATOM | 3489 | CG | LYS | 459 | 36.719 | 26.409 | 57.421 | 1.00 | 96.30 |
| ATOM | 3490 | CD | LYS | 459 | 37.458 | 27.348 | 56.475 | 1.00 | 96.73 |
| ATOM | 3491 | CE | LYS | 459 | 38.833 | 26.801 | 56.111 | 1.00 | 97.12 |
| ATOM | 3492 | NZ | LYS | 459 | 39.577 | 27.717 | 55.197 | 1.00 | 97.75 |
| ATOM | 3493 | C | LYS | 459 | 33.863 | 28.759 | 58.624 | 1.00 | 97.78 |
| ATOM | 3494 | O | LYS | 459 | 33.417 | 29.516 | 57.758 | 1.00 | 98.11 |
| ATOM | 3495 | N | ALA | 460 | 33.153 | 28.327 | 59.666 | 1.00 | 99.29 |
| ATOM | 3496 | CA | ALA | 460 | 31.778 | 28.738 | 59.916 | 1.00 | 100.54 |
| ATOM | 3497 | CB | ALA | 460 | 31.028 | 27.644 | 60.681 | 1.00 | 100.58 |
| ATOM | 3498 | C | ALA | 460 | 31.765 | 30.042 | 60.179 | 1.00 | 101.56 |
| ATOM | 3499 | O | ALA | 460 | 30.755 | 30.750 | 60.777 | 1.00 | 101.79 |
| ATOM | 3500 | N | CYS | 461 | 32.899 | 30.360 | 61.338 | 1.00 | 102.59 |
| ATOM | 3501 | CA | CYS | 461 | 33.033 | 31.572 | 62.156 | 1.00 | 103.00 |
| ATOM | 3502 | CB | CYS | 461 | 33.145 | 31.169 | 63.624 | 1.00 | 103.05 |
| ATOM | 3503 | SG | CYS | 461 | 33.354 | 32.536 | 64.774 | 1.00 | 103.32 |
| ATOM | 3504 | C | CYS | 461 | 34.265 | 32.367 | 61.753 | 1.00 | 103.21 |
| ATOM | 3505 | O | CYS | 461 | 34.788 | 33.098 | 62.620 | 1.00 | 103.54 |
| ATOM | 3506 | OXT | CYS | 461 | 34.665 | 32.248 | 60.578 | 1.00 | 103.24 |
| TER | 3507 | | CYS | 461 | | | | | |
| ATOM | 3508 | C1 | GLC | 500 | 23.469 | 1.767 | 65.521 | 1.00 | 30.82 |
| ATOM | 3509 | C2 | GLC | 500 | 23.418 | 3.122 | 64.706 | 1.00 | 29.40 |
| ATOM | 3510 | C3 | GLC | 500 | 24.837 | 3.619 | 64.445 | 1.00 | 29.78 |
| ATOM | 3511 | C4 | GLC | 500 | 25.496 | 3.860 | 65.778 | 1.00 | 28.77 |
| ATOM | 3512 | C5 | GLC | 500 | 25.529 | 2.514 | 66.593 | 1.00 | 27.72 |
| ATOM | 3513 | C6 | GLC | 500 | 26.162 | 2.717 | 67.936 | 1.00 | 26.98 |
| ATOM | 3514 | O1 | GLC | 500 | 24.127 | 0.765 | 64.857 | 1.00 | 36.62 |
| ATOM | 3515 | O2 | GLC | 500 | 22.756 | 2.872 | 63.483 | 1.00 | 32.75 |
| ATOM | 3516 | O3 | GLC | 500 | 24.786 | 4.837 | 63.698 | 1.00 | 29.31 |
| ATOM | 3517 | O4 | GLC | 500 | 26.853 | 4.253 | 65.639 | 1.00 | 29.10 |
| ATOM | 3518 | O5 | GLC | 500 | 24.152 | 2.040 | 66.770 | 1.00 | 29.59 |
| ATOM | 3519 | O6 | GLC | 500 | 25.517 | 3.687 | 68.814 | 1.00 | 30.98 |
| TER | 3520 | | GLC | 500 | | | | | |
| ATOM | 3521 | S1 | CP1 | 501 | 36.312 | 19.051 | 60.824 | 1.00 | 50.83 |
| ATOM | 3522 | C2 | CP1 | 501 | 35.720 | 19.405 | 59.240 | 1.00 | 49.96 |
| ATOM | 3523 | C3 | CP1 | 501 | 36.398 | 18.662 | 58.318 | 1.00 | 49.96 |
| ATOM | 3524 | N4 | CP1 | 501 | 37.363 | 17.829 | 58.827 | 1.00 | 49.99 |
| ATOM | 3525 | C5 | CP1 | 501 | 37.429 | 17.932 | 60.162 | 1.00 | 49.39 |
| ATOM | 3526 | N6 | CP1 | 501 | 38.317 | 17.183 | 60.878 | 1.00 | 48.07 |
| ATOM | 3527 | C7 | CP1 | 501 | 38.575 | 17.220 | 62.294 | 1.00 | 46.71 |
| ATOM | 3528 | O8 | CP1 | 501 | 37.968 | 18.001 | 63.039 | 1.00 | 47.48 |
| ATOM | 3529 | C9 | CP1 | 501 | 40.386 | 16.405 | 64.107 | 1.00 | 46.71 |
| ATOM | 3530 | C10 | CP1 | 501 | 39.620 | 16.253 | 62.884 | 1.00 | 47.34 |
| ATOM | 3531 | C11 | CP1 | 501 | 39.831 | 15.053 | 62.110 | 1.00 | 46.39 |
| ATOM | 3532 | C12 | CP1 | 501 | 40.749 | 14.066 | 62.520 | 1.00 | 46.34 |
| ATOM | 3533 | C13 | CP1 | 501 | 41.496 | 14.237 | 63.722 | 1.00 | 47.57 |
| ATOM | 3534 | F | CP1 | 501 | 42.392 | 13.310 | 64.155 | 1.00 | 48.24 |
| ATOM | 3535 | C15 | CP1 | 501 | 41.306 | 15.404 | 64.502 | 1.00 | 46.98 |
| ATOM | 3536 | S16 | CP1 | 501 | 40.907 | 12.638 | 61.485 | 1.00 | 44.61 |
| ATOM | 3537 | N17 | CP1 | 501 | 42.782 | 10.864 | 62.327 | 1.00 | 40.11 |
| ATOM | 3538 | C18 | CP1 | 501 | 42.525 | 11.942 | 61.488 | 1.00 | 41.49 |
| ATOM | 3539 | N19 | CP1 | 501 | 43.528 | 12.436 | 60.686 | 1.00 | 42.95 |
| ATOM | 3540 | C20 | CP1 | 501 | 44.549 | 11.571 | 61.054 | 1.00 | 43.00 |
| ATOM | 3541 | C21 | CP1 | 501 | 44.116 | 10.651 | 62.014 | 1.00 | 39.24 |
| ATOM | 3542 | C22 | CP1 | 501 | 41.894 | 10.152 | 63.276 | 1.00 | 32.83 |
| ATOM | 3543 | N23 | CP1 | 501 | 40.279 | 17.465 | 64.913 | 1.00 | 46.10 |
| TER | 3544 | | CP1 | 501 | JJJJ | | | | |
| ATOM | 3545 | NA + 1 | NA1 | 600 | 36.903 | 10.609 | 46.484 | 1.00 | 48.71 |
| ATOM | 3546 | O | HOH | 601 | 20.332 | −23.624 | 70.208 | 1.00 | 45.57 |
| ATOM | 3547 | O | HOH | 602 | 18.766 | −22.456 | 65.630 | 1.00 | 41.87 |
| ATOM | 3548 | O | HOH | 603 | 13.471 | −20.599 | 70.297 | 1.00 | 45.83 |
| ATOM | 3549 | O | HOH | 604 | 11.104 | −30.408 | 72.307 | 1.00 | 48.61 |
| ATOM | 3550 | O | HOH | 605 | 6.606 | −26.352 | 79.319 | 1.00 | 59.47 |
| ATOM | 3551 | O | HOH | 606 | 15.315 | −28.400 | 85.522 | 1.00 | 48.85 |
| ATOM | 3552 | O | HOH | 607 | 18.765 | −29.705 | 82.807 | 1.00 | 55.60 |
| ATOM | 3553 | O | HOH | 608 | 27.649 | −22.465 | 84.914 | 1.00 | 39.29 |
| ATOM | 3554 | O | HOH | 609 | 28.890 | −18.936 | 88.942 | 1.00 | 38.24 |
| ATOM | 3555 | O | HOH | 610 | 31.397 | −19.437 | 88.300 | 1.00 | 44.33 |
| ATOM | 3556 | O | HOH | 611 | 33.495 | −12.487 | 88.943 | 1.00 | 40.63 |
| ATOM | 3557 | O | HOH | 612 | 28.110 | −14.193 | 93.119 | 1.00 | 37.41 |
| ATOM | 3558 | O | HOH | 613 | 22.501 | −9.921 | 93.883 | 1.00 | 55.62 |
| ATOM | 3559 | O | HOH | 614 | 18.084 | −9.259 | 91.966 | 1.00 | 48.69 |
| ATOM | 3560 | O | HOH | 615 | 19.985 | −7.585 | 89.518 | 1.00 | 54.30 |
| ATOM | 3561 | O | HOH | 616 | 18.162 | −4.982 | 77.583 | 1.00 | 42.44 |
| ATOM | 3562 | O | HOH | 617 | 15.728 | −5.792 | 77.752 | 1.00 | 49.61 |
| ATOM | 3563 | O | HOH | 618 | 17.869 | −7.338 | 75.263 | 1.00 | 52.43 |
| ATOM | 3564 | O | HOH | 619 | 14.631 | −9.827 | 77.339 | 1.00 | 27.38 |
| ATOM | 3565 | O | HOH | 620 | 14.305 | −5.926 | 69.446 | 1.00 | 38.14 |
| ATOM | 3566 | O | HOH | 621 | 13.616 | −3.087 | 68.452 | 1.00 | 51.29 |
| ATOM | 3567 | O | HOH | 622 | 15.537 | −2.602 | 66.865 | 1.00 | 35.42 |
| ATOM | 3568 | O | HOH | 623 | 18.821 | −1.831 | 65.405 | 1.00 | 31.67 |
| ATOM | 3569 | O | HOH | 624 | 17.261 | 0.174 | 60.996 | 1.00 | 34.87 |
| ATOM | 3570 | O | HOH | 625 | 18.895 | −0.653 | 58.995 | 1.00 | 41.82 |
| ATOM | 3571 | O | HOH | 626 | 20.053 | −2.478 | 55.373 | 1.00 | 35.91 |
| ATOM | 3572 | O | HOH | 627 | 22.217 | −1.019 | 55.062 | 1.00 | 36.64 |
| ATOM | 3573 | O | HOH | 628 | 25.137 | −0.153 | 56.470 | 1.00 | 24.69 |
| ATOM | 3574 | O | HOH | 629 | 22.562 | 1.498 | 59.774 | 1.00 | 31.68 |
| ATOM | 3575 | O | HOH | 630 | 24.912 | 0.122 | 62.135 | 1.00 | 25.12 |
| ATOM | 3576 | O | HOH | 631 | 25.071 | 2.179 | 71.129 | 1.00 | 26.49 |
| ATOM | 3577 | O | HOH | 632 | 27.157 | 5.888 | 71.903 | 1.00 | 41.05 |
| ATOM | 3578 | O | HOH | 633 | 29.481 | 7.227 | 73.290 | 1.00 | 47.52 |
| ATOM | 3579 | O | HOH | 634 | 31.223 | 8.383 | 71.417 | 1.00 | 44.33 |
| ATOM | 3580 | O | HOH | 635 | 32.517 | 7.788 | 77.983 | 1.00 | 44.30 |
| ATOM | 3581 | O | HOH | 636 | 35.945 | 15.748 | 80.298 | 1.00 | 32.85 |
| ATOM | 3582 | O | HOH | 637 | 41.395 | 13.522 | 74.250 | 1.00 | 52.40 |
| ATOM | 3583 | O | HOH | 638 | 41.454 | 16.603 | 73.492 | 1.00 | 35.38 |
| ATOM | 3584 | O | HOH | 639 | 44.238 | 18.657 | 64.621 | 1.00 | 57.41 |
| ATOM | 3585 | O | HOH | 640 | 48.524 | 12.679 | 62.857 | 1.00 | 55.80 |
| ATOM | 3586 | O | HOH | 641 | 50.088 | 10.035 | 69.707 | 1.00 | 37.86 |
| ATOM | 3587 | O | HOH | 642 | 47.834 | 4.897 | 73.654 | 1.00 | 43.91 |
| ATOM | 3588 | O | HOH | 643 | 47.658 | 2.456 | 75.515 | 1.00 | 46.89 |
| ATOM | 3589 | O | HOH | 644 | 45.862 | 0.872 | 75.793 | 1.00 | 36.22 |
| ATOM | 3590 | O | HOH | 645 | 42.167 | −0.401 | 77.407 | 1.00 | 46.09 |
| ATOM | 3591 | O | HOH | 646 | 39.939 | −1.664 | 76.818 | 1.00 | 28.80 |
| ATOM | 3592 | O | HOH | 647 | 41.804 | 2.590 | 77.672 | 1.00 | 30.06 |
| ATOM | 3593 | O | HOH | 648 | 35.946 | −0.230 | 81.704 | 1.00 | 44.47 |
| ATOM | 3594 | O | HOH | 649 | 35.692 | −3.832 | 84.533 | 1.00 | 48.68 |
| ATOM | 3595 | O | HOH | 650 | 35.503 | −5.648 | 82.602 | 1.00 | 39.36 |
| ATOM | 3596 | O | HOH | 651 | 34.249 | −6.282 | 78.743 | 1.00 | 28.80 |
| ATOM | 3597 | O | HOH | 652 | 41.570 | −6.014 | 79.114 | 1.00 | 41.31 |
| ATOM | 3598 | O | HOH | 653 | 42.725 | −8.259 | 76.851 | 1.00 | 34.12 |
| ATOM | 3599 | O | HOH | 654 | 42.400 | −10.619 | 75.649 | 1.00 | 32.12 |
| ATOM | 3600 | O | HOH | 655 | 44.745 | −10.112 | 73.414 | 1.00 | 30.95 |
| ATOM | 3601 | O | HOH | 656 | 44.977 | −6.287 | 75.709 | 1.00 | 54.82 |
| ATOM | 3602 | O | HOH | 657 | 49.536 | −3.896 | 71.639 | 1.00 | 46.68 |
| ATOM | 3603 | O | HOH | 658 | 47.500 | −6.424 | 68.659 | 1.00 | 37.00 |
| ATOM | 3604 | O | HOH | 659 | 46.887 | −8.289 | 65.948 | 1.00 | 35.73 |
| ATOM | 3605 | O | HOH | 660 | 45.007 | −14.004 | 70.403 | 1.00 | 31.53 |
| ATOM | 3606 | O | HOH | 661 | 44.785 | −16.666 | 70.958 | 1.00 | 39.67 |
| ATOM | 3607 | O | HOH | 662 | 39.546 | −15.899 | 74.666 | 1.00 | 38.86 |
| ATOM | 3608 | O | HOH | 663 | 38.539 | −14.985 | 72.232 | 1.00 | 34.80 |
| ATOM | 3609 | O | HOH | 664 | 38.252 | −17.032 | 68.208 | 1.00 | 47.76 |
| ATOM | 3610 | O | HOH | 665 | 39.836 | −15.454 | 66.437 | 1.00 | 38.55 |
| ATOM | 3611 | O | HOH | 666 | 36.975 | −19.549 | 67.636 | 1.00 | 43.12 |
| ATOM | 3612 | O | HOH | 667 | 37.200 | −20.262 | 70.388 | 1.00 | 51.64 |
| ATOM | 3613 | O | HOH | 668 | 33.328 | −20.695 | 70.543 | 1.00 | 49.91 |
| ATOM | 3614 | O | HOH | 669 | 32.877 | −18.716 | 69.209 | 1.00 | 30.69 |
| ATOM | 3615 | O | HOH | 670 | 30.463 | −18.228 | 69.770 | 1.00 | 29.35 |
| ATOM | 3616 | O | HOH | 671 | 29.403 | −18.862 | 72.028 | 1.00 | 29.94 |
| ATOM | 3617 | O | HOH | 672 | 31.677 | −19.876 | 75.929 | 1.00 | 57.83 |
| ATOM | 3618 | O | HOH | 673 | 32.105 | −15.120 | 81.811 | 1.00 | 56.36 |
| ATOM | 3619 | O | HOH | 674 | 25.408 | −13.262 | 70.399 | 1.00 | 19.73 |
| ATOM | 3620 | O | HOH | 675 | 20.199 | −11.770 | 66.567 | 1.00 | 31.95 |
| ATOM | 3621 | O | HOH | 676 | 20.589 | −11.169 | 63.684 | 1.00 | 28.18 |
| ATOM | 3622 | O | HOH | 677 | 18.416 | −12.169 | 62.695 | 1.00 | 34.73 |
| ATOM | 3623 | O | HOH | 678 | 18.037 | −12.657 | 56.097 | 1.00 | 62.31 |
| ATOM | 3624 | O | HOH | 679 | 15.700 | −10.616 | 55.942 | 1.00 | 49.61 |
| ATOM | 3625 | O | HOH | 680 | 17.485 | −8.240 | 55.372 | 1.00 | 37.91 |
| ATOM | 3626 | O | HOH | 681 | 22.370 | −12.555 | 56.733 | 1.00 | 27.53 |
| ATOM | 3627 | O | HOH | 682 | 21.048 | −16.039 | 51.265 | 1.00 | 53.09 |
| ATOM | 3628 | O | HOH | 683 | 25.649 | −8.890 | 49.620 | 1.00 | 43.30 |
| ATOM | 3629 | O | HOH | 684 | 25.472 | −5.908 | 50.031 | 1.00 | 43.23 |
| ATOM | 3630 | O | HOH | 685 | 27.841 | −3.633 | 51.119 | 1.00 | 34.64 |
| ATOM | 3631 | O | HOH | 686 | 23.209 | 1.359 | 50.792 | 1.00 | 44.06 |
| ATOM | 3632 | O | HOH | 687 | 26.198 | 3.711 | 50.151 | 1.00 | 38.65 |
| ATOM | 3633 | O | HOH | 688 | 27.728 | 6.416 | 50.494 | 1.00 | 39.66 |
| ATOM | 3634 | O | HOH | 689 | 30.171 | 5.238 | 50.152 | 1.00 | 36.90 |
| ATOM | 3635 | O | HOH | 690 | 32.248 | 6.334 | 48.750 | 1.00 | 33.36 |
| ATOM | 3636 | O | HOH | 691 | 36.665 | 2.495 | 46.196 | 1.00 | 32.68 |
| ATOM | 3637 | O | HOH | 692 | 37.821 | 0.573 | 47.634 | 1.00 | 47.42 |
| ATOM | 3638 | O | HOH | 693 | 42.794 | 0.201 | 52.097 | 1.00 | 44.65 |
| ATOM | 3639 | O | HOH | 694 | 41.559 | 1.725 | 53.810 | 1.00 | 38.52 |
| ATOM | 3640 | O | HOH | 695 | 43.105 | 3.662 | 55.242 | 1.00 | 34.89 |
| ATOM | 3641 | O | HOH | 696 | 45.510 | 2.836 | 56.086 | 1.00 | 40.92 |
| ATOM | 3642 | O | HOH | 697 | 50.206 | 2.510 | 60.598 | 1.00 | 45.86 |
| ATOM | 3643 | O | HOH | 698 | 52.258 | 1.308 | 61.720 | 1.00 | 45.43 |
| ATOM | 3644 | O | HOH | 699 | 48.954 | 1.961 | 67.618 | 1.00 | 35.43 |
| ATOM | 3645 | O | HOH | 700 | 49.694 | −0.399 | 68.442 | 1.00 | 39.38 |
| ATOM | 3646 | O | HOH | 701 | 40.015 | −5.106 | 51.960 | 1.00 | 36.49 |
| ATOM | 3647 | O | HOH | 702 | 34.048 | −12.903 | 50.839 | 1.00 | 37.87 |
| ATOM | 3648 | O | HOH | 703 | 33.190 | −14.541 | 52.882 | 1.00 | 51.09 |
| ATOM | 3649 | O | HOH | 704 | 34.961 | −16.254 | 52.067 | 1.00 | 35.42 |
| ATOM | 3650 | O | HOH | 705 | 30.397 | −15.105 | 52.902 | 1.00 | 39.69 |
| ATOM | 3651 | O | HOH | 706 | 31.770 | −20.985 | 57.467 | 1.00 | 48.16 |
| ATOM | 3652 | O | HOH | 707 | 37.192 | −19.637 | 55.866 | 1.00 | 46.43 |
| ATOM | 3653 | O | HOH | 708 | 38.187 | −23.567 | 61.924 | 1.00 | 40.92 |
| ATOM | 3654 | O | HOH | 709 | 38.470 | −23.126 | 65.456 | 1.00 | 45.43 |
| ATOM | 3655 | O | HOH | 710 | 30.533 | −23.844 | 62.578 | 1.00 | 37.90 |
| ATOM | 3656 | O | HOH | 711 | 26.515 | −21.678 | 62.544 | 1.00 | 39.08 |
| ATOM | 3657 | O | HOH | 712 | 27.242 | −20.400 | 65.671 | 1.00 | 33.60 |
| ATOM | 3658 | O | HOH | 713 | 25.907 | −18.116 | 65.171 | 1.00 | 24.64 |
| ATOM | 3659 | O | HOH | 714 | 28.226 | −26.567 | 74.622 | 1.00 | 44.93 |
| ATOM | 3660 | O | HOH | 715 | 31.091 | −28.151 | 73.632 | 1.00 | 39.43 |
| ATOM | 3661 | O | HOH | 716 | 28.020 | −32.685 | 74.512 | 1.00 | 48.35 |
| ATOM | 3662 | O | HOH | 717 | 28.401 | −36.363 | 77.956 | 1.00 | 47.24 |
| ATOM | 3663 | O | HOH | 718 | 26.796 | −22.733 | 95.375 | 1.00 | 34.50 |
| ATOM | 3664 | O | HOH | 719 | 23.506 | −18.729 | 96.532 | 1.00 | 46.50 |
| ATOM | 3665 | O | HOH | 720 | 7.193 | −13.392 | 87.134 | 1.00 | 48.33 |
| ATOM | 3666 | O | HOH | 721 | 23.769 | −2.393 | 77.130 | 1.00 | 39.79 |
| ATOM | 3667 | O | HOH | 722 | 21.538 | 6.141 | 76.432 | 1.00 | 52.58 |
| ATOM | 3668 | O | HOH | 723 | 26.038 | 13.552 | 80.579 | 1.00 | 47.60 |
| ATOM | 3669 | O | HOH | 724 | 25.460 | 9.823 | 62.329 | 1.00 | 33.10 |
| ATOM | 3670 | O | HOH | 725 | 27.321 | 10.443 | 60.403 | 1.00 | 39.23 |
| ATOM | 3671 | O | HOH | 726 | 26.658 | 8.602 | 58.871 | 1.00 | 32.16 |
| ATOM | 3672 | O | HOH | 727 | 29.670 | 11.059 | 61.417 | 1.00 | 24.95 |
| ATOM | 3673 | O | HOH | 728 | 30.585 | 13.937 | 60.932 | 1.00 | 41.90 |
| ATOM | 3674 | O | HOH | 729 | 34.591 | 18.790 | 55.094 | 1.00 | 40.47 |
| ATOM | 3675 | O | HOH | 730 | 34.117 | 19.353 | 52.182 | 1.00 | 54.62 |
| ATOM | 3676 | O | HOH | 731 | 31.428 | 16.535 | 48.224 | 1.00 | 37.06 |
| ATOM | 3677 | O | HOH | 732 | 31.432 | 15.488 | 46.047 | 1.00 | 33.85 |
| ATOM | 3678 | O | HOH | 733 | 27.660 | 11.291 | 51.289 | 1.00 | 40.74 |
| ATOM | 3679 | O | HOH | 734 | 27.629 | 10.029 | 53.857 | 1.00 | 30.56 |
| ATOM | 3680 | O | HOH | 735 | 22.996 | 7.311 | 45.724 | 1.00 | 57.65 |
| ATOM | 3681 | O | HOH | 736 | 25.532 | 2.038 | 43.263 | 1.00 | 34.43 |
| ATOM | 3682 | O | HOH | 737 | 33.508 | 3.221 | 40.211 | 1.00 | 45.05 |
| ATOM | 3683 | O | HOH | 738 | 35.525 | 1.426 | 41.242 | 1.00 | 44.71 |
| ATOM | 3684 | O | HOH | 739 | 37.227 | 9.576 | 44.352 | 1.00 | 31.96 |
| ATOM | 3685 | O | HOH | 740 | 39.858 | 15.804 | 52.237 | 1.00 | 43.41 |
| ATOM | 3686 | O | HOH | 741 | 42.053 | 15.415 | 53.940 | 1.00 | 47.39 |
| ATOM | 3687 | O | HOH | 742 | 32.200 | 24.148 | 58.683 | 1.00 | 45.42 |
| ATOM | 3688 | O | HOH | 743 | 28.016 | 21.804 | 51.201 | 1.00 | 44.12 |
| ATOM | 3689 | O | HOH | 744 | 22.797 | 26.498 | 63.763 | 1.00 | 53.69 |
| ATOM | 3690 | O | HOH | 745 | 10.552 | 26.073 | 62.119 | 1.00 | 43.13 |
| ATOM | 3691 | O | HOH | 746 | 11.190 | 7.673 | 68.338 | 1.00 | 57.06 |
| ATOM | 3692 | O | HOH | 747 | 20.818 | −3.881 | 51.225 | 1.00 | 56.55 |
| ATOM | 3693 | O | HOH | 748 | 29.885 | −6.633 | 43.981 | 1.00 | 46.17 |
| ATOM | 3694 | O | HOH | 749 | 40.811 | 30.945 | 68.309 | 1.00 | 45.88 |
| TER | 3695 | | HOH | | | | | | |

Table 1 is made according to a description method of Protein Data Bank conventionally used by those skilled in th art. In Table 1, GLC represents a glucose molecule, CP1 represents a compound of the formula IIIa, and HOH represents a water molecule.

The present invention has succeeded in preparing a crystal of a GK protein as depicted in SEQ ID No. 8 (see, examples below). Thus obtained crystal of a GK protein had a lattice constant satisfying the following formulae (5) to (8):
a=b=103.2±5 Å (5)
c=281.0±7 Å (6)
α=β=90° (7)
γ=120° (8)
This crystal was analyzed to have a space group of P6₅22. Here, the above-mentioned a=b is preferably 103.2±3 Å, more preferably 103.2±2 Å, further preferably 103.2±1 Å. The above-mentioned c is preferably 281.0±6 Å, more preferably 281.0±4 Å, further preferably 281.0±2 Å.

The three dimensional structure coordinate of thus obtained GK protein crystal is shown in Table 2.

TABLE 2

| | | | | | | | | | |
|---|---|---|---|---|---|---|---|---|---|
| ATOM | 1 | CB | MET | 15 | 54.150 | 5.972 | 67.103 | 1.00 | 55.10 |
| ATOM | 2 | CG | MET | 15 | 55.594 | 5.943 | 67.591 | 1.00 | 55.46 |
| ATOM | 3 | SD | MET | 15 | 56.013 | 4.505 | 68.603 | 1.00 | 52.92 |
| ATOM | 4 | CE | MET | 15 | 56.517 | 5.326 | 70.108 | 1.00 | 51.73 |
| ATOM | 5 | C | MET | 15 | 52.357 | 4.955 | 65.669 | 1.00 | 56.87 |
| ATOM | 6 | O | MET | 15 | 52.057 | 4.609 | 64.524 | 1.00 | 57.60 |
| ATOM | 7 | N | MET | 15 | 54.770 | 4.766 | 65.028 | 1.00 | 55.00 |
| ATOM | 8 | CA | MET | 15 | 53.800 | 4.813 | 66.167 | 1.00 | 56.04 |
| ATOM | 9 | N | VAL | 16 | 51.468 | 5.456 | 66.525 | 1.00 | 55.58 |
| ATOM | 10 | CA | VAL | 16 | 50.065 | 5.625 | 66.154 | 1.00 | 52.87 |
| ATOM | 11 | CB | VAL | 16 | 49.141 | 4.862 | 67.129 | 1.00 | 49.32 |
| ATOM | 12 | CG1 | VAL | 16 | 47.696 | 5.016 | 66.716 | 1.00 | 48.26 |
| ATOM | 13 | CG2 | VAL | 16 | 49.508 | 3.394 | 67.126 | 1.00 | 47.28 |
| ATOM | 14 | C | VAL | 16 | 49.666 | 7.097 | 66.085 | 1.00 | 53.26 |
| ATOM | 15 | O | VAL | 16 | 49.218 | 7.563 | 65.040 | 1.00 | 52.32 |
| ATOM | 16 | N | GLU | 17 | 49.845 | 7.828 | 67.182 | 1.00 | 56.12 |
| ATOM | 17 | CA | GLU | 17 | 49.511 | 9.253 | 67.210 | 1.00 | 59.41 |
| ATOM | 18 | CB | GLU | 17 | 50.102 | 9.921 | 68.456 | 1.00 | 63.35 |
| ATOM | 19 | CG | GLU | 17 | 49.063 | 10.373 | 69.484 | 1.00 | 68.69 |
| ATOM | 20 | CD | GLU | 17 | 48.174 | 11.525 | 69.004 | 1.00 | 72.00 |
| ATOM | 21 | OE1 | GLU | 17 | 47.314 | 11.964 | 69.805 | 1.00 | 74.22 |
| ATOM | 22 | OE2 | GLU | 17 | 48.328 | 11.992 | 67.847 | 1.00 | 72.36 |
| ATOM | 23 | C | GLU | 17 | 50.035 | 9.963 | 65.967 | 1.00 | 59.05 |
| ATOM | 24 | O | GLU | 17 | 49.521 | 11.011 | 65.566 | 1.00 | 57.70 |
| ATOM | 25 | N | GLN | 18 | 51.070 | 9.389 | 65.367 | 1.00 | 60.75 |
| ATOM | 26 | CA | GLN | 18 | 51.661 | 9.960 | 64.170 | 1.00 | 61.70 |
| ATOM | 27 | CB | GLN | 18 | 53.038 | 9.329 | 63.895 | 1.00 | 66.55 |
| ATOM | 28 | CG | GLN | 18 | 54.001 | 9.219 | 65.110 | 1.00 | 72.22 |
| ATOM | 29 | CD | GLN | 18 | 54.509 | 10.566 | 65.654 | 1.00 | 75.87 |
| ATOM | 30 | OE1 | GLN | 18 | 55.317 | 10.605 | 66.595 | 1.00 | 75.55 |
| ATOM | 31 | NE2 | GLN | 18 | 54.037 | 11.669 | 65.067 | 1.00 | 77.63 |
| ATOM | 32 | C | GLN | 18 | 50.709 | 9.682 | 63.004 | 1.00 | 59.33 |
| ATOM | 33 | O | GLN | 18 | 50.322 | 10.601 | 62.287 | 1.00 | 59.09 |
| ATOM | 34 | N | ILE | 19 | 50.321 | 8.418 | 62.832 | 1.00 | 55.64 |
| ATOM | 35 | CA | ILE | 19 | 49.416 | 8.029 | 61.747 | 1.00 | 53.41 |
| ATOM | 36 | CB | ILE | 19 | 49.113 | 6.529 | 61.778 | 1.00 | 52.34 |
| ATOM | 37 | CG2 | ILE | 19 | 47.964 | 6.211 | 60.832 | 1.00 | 50.69 |
| ATOM | 38 | CG1 | ILE | 19 | 50.374 | 5.754 | 61.389 | 1.00 | 52.73 |
| ATOM | 39 | CD1 | ILE | 19 | 50.186 | 4.256 | 61.274 | 1.00 | 53.73 |
| ATOM | 40 | C | ILE | 19 | 48.088 | 8.774 | 61.741 | 1.00 | 53.03 |
| ATOM | 41 | O | ILE | 19 | 47.791 | 9.528 | 60.812 | 1.00 | 52.86 |
| ATOM | 42 | N | LEU | 20 | 47.279 | 8.548 | 62.766 | 1.00 | 52.38 |
| ATOM | 43 | CA | LEU | 20 | 45.997 | 9.228 | 62.861 | 1.00 | 51.95 |
| ATOM | 44 | CB | LEU | 20 | 45.336 | 8.937 | 64.195 | 1.00 | 50.70 |
| ATOM | 45 | CG | LEU | 20 | 44.563 | 7.632 | 64.212 | 1.00 | 51.65 |
| ATOM | 46 | CD1 | LEU | 20 | 45.450 | 6.454 | 63.803 | 1.00 | 51.77 |
| ATOM | 47 | CD2 | LEU | 20 | 44.010 | 7.463 | 65.599 | 1.00 | 51.02 |
| ATOM | 48 | C | LEU | 20 | 46.158 | 10.723 | 62.727 | 1.00 | 52.33 |
| ATOM | 49 | O | LEU | 20 | 45.204 | 11.427 | 62.401 | 1.00 | 54.11 |
| ATOM | 50 | N | ALA | 21 | 47.366 | 11.207 | 62.990 | 1.00 | 51.49 |
| ATOM | 51 | CA | ALA | 21 | 47.643 | 12.628 | 62.907 | 1.00 | 49.87 |
| ATOM | 52 | CB | ALA | 21 | 49.066 | 12.899 | 63.342 | 1.00 | 50.58 |
| ATOM | 53 | C | ALA | 21 | 47.414 | 13.133 | 61.491 | 1.00 | 48.63 |
| ATOM | 54 | O | ALA | 21 | 47.090 | 14.301 | 61.286 | 1.00 | 47.74 |
| ATOM | 55 | N | GLU | 22 | 47.571 | 12.243 | 60.517 | 1.00 | 47.60 |
| ATOM | 56 | CA | GLU | 22 | 47.383 | 12.605 | 59.121 | 1.00 | 48.69 |
| ATOM | 57 | CB | GLU | 22 | 47.818 | 11.457 | 58.215 | 1.00 | 51.49 |
| ATOM | 58 | CG | GLU | 22 | 49.282 | 11.520 | 57.838 | 1.00 | 59.47 |
| ATOM | 59 | CD | GLU | 22 | 49.738 | 10.335 | 57.003 | 1.00 | 64.78 |
| ATOM | 60 | OE1 | GLU | 22 | 50.896 | 10.369 | 56.519 | 1.00 | 66.47 |
| ATOM | 61 | OE2 | GLU | 22 | 48.948 | 9.373 | 56.839 | 1.00 | 68.05 |
| ATOM | 62 | C | GLU | 22 | 45.954 | 12.999 | 58.794 | 1.00 | 48.26 |
| ATOM | 63 | O | GLU | 22 | 45.683 | 13.538 | 57.721 | 1.00 | 48.86 |
| ATOM | 64 | N | PHE | 23 | 45.036 | 12.733 | 59.715 | 1.00 | 47.14 |
| ATOM | 65 | CA | PHE | 23 | 43.641 | 13.076 | 59.490 | 1.00 | 45.51 |
| ATOM | 66 | CB | PHE | 23 | 42.722 | 12.045 | 60.147 | 1.00 | 41.36 |
| ATOM | 67 | CG | PHE | 23 | 42.544 | 10.783 | 59.347 | 1.00 | 37.96 |
| ATOM | 68 | CD1 | PHE | 23 | 43.208 | 9.613 | 59.697 | 1.00 | 35.23 |
| ATOM | 69 | CD2 | PHE | 23 | 41.687 | 10.758 | 58.255 | 1.00 | 37.67 |
| ATOM | 70 | CE1 | PHE | 23 | 43.016 | 8.435 | 58.968 | 1.00 | 32.67 |
| ATOM | 71 | CE2 | PHE | 23 | 41.492 | 9.583 | 57.523 | 1.00 | 37.15 |
| ATOM | 72 | CZ | PHE | 23 | 42.158 | 8.423 | 57.883 | 1.00 | 33.48 |
| ATOM | 73 | C | PHE | 23 | 43.310 | 14.468 | 60.013 | 1.00 | 47.24 |
| ATOM | 74 | O | PHE | 23 | 42.227 | 14.993 | 59.767 | 1.00 | 46.34 |
| ATOM | 75 | N | GLN | 24 | 44.245 | 15.068 | 60.735 | 1.00 | 50.44 |
| ATOM | 76 | CA | GLN | 24 | 44.028 | 16.400 | 61.279 | 1.00 | 55.06 |
| ATOM | 77 | CB | GLN | 24 | 45.306 | 16.882 | 61.979 | 1.00 | 59.10 |
| ATOM | 78 | CG | GLN | 24 | 45.715 | 16.023 | 63.168 | 1.00 | 62.03 |
| ATOM | 79 | CD | GLN | 24 | 44.686 | 16.075 | 64.277 | 1.00 | 65.56 |
| ATOM | 80 | OE1 | GLN | 24 | 44.653 | 15.207 | 65.156 | 1.00 | 66.95 |
| ATOM | 81 | NE2 | GLN | 24 | 43.834 | 17.103 | 64.245 | 1.00 | 65.89 |
| ATOM | 82 | C | GLN | 24 | 43.644 | 17.359 | 60.149 | 1.00 | 56.09 |
| ATOM | 83 | O | GLN | 24 | 43.892 | 17.073 | 58.979 | 1.00 | 57.63 |
| ATOM | 84 | N | LEU | 25 | 43.016 | 18.476 | 60.504 | 1.00 | 55.99 |
| ATOM | 85 | CA | LEU | 25 | 42.616 | 19.501 | 59.540 | 1.00 | 55.27 |
| ATOM | 86 | CB | LEU | 25 | 41.303 | 19.128 | 58.841 | 1.00 | 54.71 |
| ATOM | 87 | CG | LEU | 25 | 41.325 | 17.896 | 57.922 | 1.00 | 53.30 |
| ATOM | 88 | CD1 | LEU | 25 | 39.928 | 17.618 | 57.419 | 1.00 | 53.18 |
| ATOM | 89 | CD2 | LEU | 25 | 42.264 | 18.113 | 56.755 | 1.00 | 51.55 |
| ATOM | 90 | C | LEU | 25 | 42.444 | 20.786 | 60.336 | 1.00 | 56.31 |
| ATOM | 91 | O | LEU | 25 | 41.377 | 21.061 | 60.889 | 1.00 | 55.85 |
| ATOM | 92 | N | GLN | 26 | 43.519 | 21.563 | 60.399 | 1.00 | 58.22 |
| ATOM | 93 | CA | GLN | 26 | 43.527 | 22.807 | 61.153 | 1.00 | 58.31 |
| ATOM | 94 | CB | GLN | 26 | 44.980 | 23.280 | 61.361 | 1.00 | 63.03 |
| ATOM | 95 | CG | GLN | 26 | 45.118 | 24.480 | 62.313 | 1.00 | 69.87 |
| ATOM | 96 | CD | GLN | 26 | 46.490 | 25.161 | 62.245 | 1.00 | 73.70 |
| ATOM | 97 | OE1 | GLN | 26 | 47.009 | 25.446 | 61.158 | 1.00 | 74.68 |
| ATOM | 98 | NE2 | GLN | 26 | 47.067 | 25.446 | 63.411 | 1.00 | 74.99 |
| ATOM | 99 | C | GLN | 26 | 42.702 | 23.903 | 60.485 | 1.00 | 55.29 |
| ATOM | 100 | O | GLN | 26 | 42.358 | 23.811 | 59.308 | 1.00 | 51.30 |
| ATOM | 101 | N | GLU | 27 | 42.389 | 24.931 | 61.267 | 1.00 | 55.08 |
| ATOM | 102 | CA | GLU | 27 | 41.617 | 26.083 | 60.824 | 1.00 | 55.66 |
| ATOM | 103 | CB | GLU | 27 | 41.940 | 27.280 | 61.709 | 1.00 | 57.13 |
| ATOM | 104 | CG | GLU | 27 | 41.029 | 28.469 | 61.523 | 1.00 | 59.64 |
| ATOM | 105 | CD | GLU | 27 | 39.694 | 28.272 | 62.208 | 1.00 | 62.00 |
| ATOM | 106 | OE1 | GLU | 27 | 39.685 | 27.840 | 63.382 | 1.00 | 62.44 |
| ATOM | 107 | OE2 | GLU | 27 | 38.653 | 28.559 | 61.581 | 1.00 | 64.27 |
| ATOM | 108 | C | GLU | 27 | 41.905 | 26.454 | 59.380 | 1.00 | 55.70 |
| ATOM | 109 | O | GLU | 27 | 41.025 | 26.416 | 58.531 | 1.00 | 56.30 |
| ATOM | 110 | N | GLU | 28 | 43.147 | 26.828 | 59.113 | 1.00 | 56.74 |
| ATOM | 111 | CA | GLU | 28 | 43.571 | 27.208 | 57.770 | 1.00 | 58.34 |
| ATOM | 112 | CB | GLU | 28 | 45.102 | 27.226 | 57.714 | 1.00 | 63.94 |
| ATOM | 113 | CG | GLU | 28 | 45.704 | 28.026 | 56.573 | 1.00 | 70.36 |
| ATOM | 114 | CD | GLU | 28 | 45.615 | 29.524 | 56.806 | 1.00 | 74.74 |
| ATOM | 115 | OE1 | GLU | 28 | 46.245 | 30.289 | 56.040 | 1.00 | 77.18 |
| ATOM | 116 | OE2 | GLU | 28 | 44.912 | 29.938 | 57.755 | 1.00 | 77.44 |
| ATOM | 117 | C | GLU | 28 | 43.032 | 26.231 | 56.721 | 1.00 | 56.56 |
| ATOM | 118 | O | GLU | 28 | 42.375 | 26.641 | 55.764 | 1.00 | 54.38 |
| ATOM | 119 | N | ASP | 29 | 43.316 | 24.942 | 56.921 | 1.00 | 55.20 |
| ATOM | 120 | CA | ASP | 29 | 42.893 | 23.869 | 56.015 | 1.00 | 53.13 |
| ATOM | 121 | CB | ASP | 29 | 43.106 | 22.499 | 56.667 | 1.00 | 56.36 |
| ATOM | 122 | CG | ASP | 29 | 44.570 | 22.116 | 56.758 | 1.00 | 59.69 |
| ATOM | 123 | OD1 | ASP | 29 | 45.263 | 22.198 | 55.717 | 1.00 | 61.07 |
| ATOM | 124 | OD2 | ASP | 29 | 45.021 | 21.727 | 57.863 | 1.00 | 60.92 |
| ATOM | 125 | C | ASP | 29 | 41.439 | 23.995 | 55.607 | 1.00 | 49.74 |
| ATOM | 126 | O | ASP | 29 | 41.100 | 23.924 | 54.424 | 1.00 | 47.81 |
| ATOM | 127 | N | LEU | 30 | 40.579 | 24.156 | 56.603 | 1.00 | 46.04 |
| ATOM | 128 | CA | LEU | 30 | 39.167 | 24.309 | 56.344 | 1.00 | 43.06 |
| ATOM | 129 | CB | LEU | 30 | 38.393 | 24.491 | 57.649 | 1.00 | 39.08 |
| ATOM | 130 | CG | LEU | 30 | 38.026 | 23.218 | 58.404 | 1.00 | 36.61 |
| ATOM | 131 | CD1 | LEU | 30 | 39.280 | 22.441 | 58.756 | 1.00 | 37.28 |
| ATOM | 132 | CD2 | LEU | 30 | 37.233 | 23.576 | 59.642 | 1.00 | 35.29 |
| ATOM | 133 | C | LEU | 30 | 38.948 | 25.516 | 55.452 | 1.00 | 44.18 |
| ATOM | 134 | O | LEU | 30 | 38.410 | 25.388 | 54.354 | 1.00 | 45.60 |
| ATOM | 135 | N | LYS | 31 | 39.381 | 26.685 | 55.920 | 1.00 | 44.63 |
| ATOM | 136 | CA | LYS | 31 | 39.206 | 27.927 | 55.170 | 1.00 | 43.67 |
| ATOM | 137 | CB | LYS | 31 | 40.136 | 29.020 | 55.695 | 1.00 | 45.23 |
| ATOM | 138 | CG | LYS | 31 | 39.968 | 29.361 | 57.165 | 1.00 | 46.98 |
| ATOM | 139 | CD | LYS | 31 | 38.743 | 30.221 | 57.440 | 1.00 | 45.54 |
| ATOM | 140 | CE | LYS | 31 | 38.695 | 30.675 | 58.915 | 1.00 | 45.82 |
| ATOM | 141 | NZ | LYS | 31 | 39.836 | 31.545 | 59.387 | 1.00 | 42.73 |
| ATOM | 142 | C | LYS | 31 | 39.483 | 27.725 | 53.697 | 1.00 | 42.23 |
| ATOM | 143 | O | LYS | 31 | 38.759 | 28.241 | 52.855 | 1.00 | 41.29 |
| ATOM | 144 | N | LYS | 32 | 40.535 | 26.976 | 53.385 | 1.00 | 41.79 |
| ATOM | 145 | CA | LYS | 32 | 40.877 | 26.737 | 51.994 | 1.00 | 43.47 |
| ATOM | 146 | CB | LYS | 32 | 42.171 | 25.928 | 51.888 | 1.00 | 45.16 |
| ATOM | 147 | CG | LYS | 32 | 42.811 | 25.974 | 50.499 | 1.00 | 50.49 |
| ATOM | 148 | CD | LYS | 32 | 44.302 | 25.565 | 50.510 | 1.00 | 54.48 |
| ATOM | 149 | CE | LYS | 32 | 44.505 | 24.086 | 50.900 | 1.00 | 57.45 |
| ATOM | 150 | NZ | LYS | 32 | 45.934 | 23.610 | 51.002 | 1.00 | 56.65 |
| ATOM | 151 | C | LYS | 32 | 39.740 | 25.995 | 51.308 | 1.00 | 43.99 |
| ATOM | 152 | O | LYS | 32 | 39.260 | 26.407 | 50.246 | 1.00 | 43.34 |
| ATOM | 153 | N | VAL | 33 | 39.306 | 24.901 | 51.925 | 1.00 | 43.47 |
| ATOM | 154 | CA | VAL | 33 | 38.218 | 24.100 | 51.382 | 1.00 | 40.87 |
| ATOM | 155 | CB | VAL | 33 | 37.895 | 22.927 | 52.310 | 1.00 | 40.53 |
| ATOM | 156 | CG1 | VAL | 33 | 36.977 | 21.939 | 51.604 | 1.00 | 40.20 |
| ATOM | 157 | CG2 | VAL | 33 | 39.183 | 22.248 | 52.729 | 1.00 | 40.29 |
| ATOM | 158 | C | VAL | 33 | 36.994 | 24.981 | 51.226 | 1.00 | 39.39 |
| ATOM | 159 | O | VAL | 33 | 36.370 | 25.011 | 50.165 | 1.00 | 37.22 |
| ATOM | 160 | N | MET | 34 | 36.675 | 25.707 | 52.290 | 1.00 | 39.46 |
| ATOM | 161 | CA | MET | 34 | 35.539 | 26.609 | 52.288 | 1.00 | 42.17 |
| ATOM | 162 | CB | MET | 34 | 35.515 | 27.460 | 53.555 | 1.00 | 43.81 |
| ATOM | 163 | CG | MET | 34 | 34.259 | 28.305 | 53.656 | 1.00 | 48.81 |
| ATOM | 164 | SD | MET | 34 | 34.302 | 29.606 | 54.908 | 1.00 | 56.60 |
| ATOM | 165 | CE | MET | 34 | 34.576 | 31.074 | 53.859 | 1.00 | 55.54 |
| ATOM | 166 | C | MET | 34 | 35.612 | 27.535 | 51.086 | 1.00 | 43.35 |
| ATOM | 167 | O | MET | 34 | 34.626 | 27.735 | 50.383 | 1.00 | 43.86 |
| ATOM | 168 | N | ARG | 35 | 36.785 | 28.104 | 50.847 | 1.00 | 44.90 |
| ATOM | 169 | CA | ARG | 35 | 36.938 | 29.015 | 49.729 | 1.00 | 45.60 |
| ATOM | 170 | CB | ARG | 35 | 38.286 | 29.727 | 49.815 | 1.00 | 49.40 |
| ATOM | 171 | CG | ARG | 35 | 38.459 | 30.563 | 51.075 | 1.00 | 53.81 |
| ATOM | 172 | CD | ARG | 35 | 38.231 | 32.052 | 50.851 | 1.00 | 57.78 |
| ATOM | 173 | NE | ARG | 35 | 38.483 | 32.807 | 52.077 | 1.00 | 63.20 |
| ATOM | 174 | CZ | ARG | 35 | 39.587 | 32.696 | 52.820 | 1.00 | 65.30 |
| ATOM | 175 | NH1 | ARG | 35 | 40.557 | 31.854 | 52.466 | 1.00 | 64.80 |
| ATOM | 176 | NH2 | ARG | 35 | 39.720 | 33.425 | 53.925 | 1.00 | 66.89 |
| ATOM | 177 | C | ARG | 35 | 36.814 | 28.262 | 48.418 | 1.00 | 44.08 |
| ATOM | 178 | O | ARG | 35 | 35.977 | 28.605 | 47.586 | 1.00 | 43.75 |
| ATOM | 179 | N | ARG | 36 | 37.633 | 27.227 | 48.245 | 1.00 | 43.43 |
| ATOM | 180 | CA | ARG | 36 | 37.612 | 26.418 | 47.026 | 1.00 | 43.94 |
| ATOM | 181 | CB | ARG | 36 | 38.547 | 25.212 | 47.174 | 1.00 | 44.76 |
| ATOM | 182 | CG | ARG | 36 | 40.020 | 25.580 | 47.244 | 1.00 | 44.66 |
| ATOM | 183 | CD | ARG | 36 | 40.898 | 24.392 | 47.617 | 1.00 | 44.20 |
| ATOM | 184 | NE | ARG | 36 | 41.728 | 23.919 | 46.512 | 1.00 | 44.66 |
| ATOM | 185 | CZ | ARG | 36 | 42.890 | 23.292 | 46.678 | 1.00 | 45.10 |
| ATOM | 186 | NH1 | ARG | 36 | 43.350 | 23.075 | 47.900 | 1.00 | 44.34 |
| ATOM | 187 | NH2 | ARG | 36 | 43.590 | 22.870 | 45.631 | 1.00 | 45.47 |
| ATOM | 188 | C | ARG | 36 | 36.202 | 25.941 | 46.660 | 1.00 | 43.73 |
| ATOM | 189 | O | ARG | 36 | 35.921 | 25.645 | 45.497 | 1.00 | 43.31 |
| ATOM | 190 | N | MET | 37 | 35.324 | 25.851 | 47.656 | 1.00 | 42.87 |
| ATOM | 191 | CA | MET | 37 | 33.946 | 25.440 | 47.413 | 1.00 | 41.30 |
| ATOM | 192 | CB | MET | 37 | 33.222 | 25.136 | 48.726 | 1.00 | 43.30 |
| ATOM | 193 | CG | MET | 37 | 31.782 | 24.636 | 48.556 | 1.00 | 45.16 |
| ATOM | 194 | SD | MET | 37 | 31.646 | 22.826 | 48.280 | 1.00 | 52.61 |
| ATOM | 195 | CE | MET | 37 | 31.892 | 22.708 | 46.492 | 1.00 | 46.47 |
| ATOM | 196 | C | MET | 37 | 33.249 | 26.603 | 46.723 | 1.00 | 39.52 |
| ATOM | 197 | O | MET | 37 | 32.702 | 26.458 | 45.635 | 1.00 | 39.06 |
| ATOM | 198 | N | GLN | 38 | 33.275 | 27.767 | 47.359 | 1.00 | 37.22 |
| ATOM | 199 | CA | GLN | 38 | 32.637 | 28.927 | 46.776 | 1.00 | 35.67 |
| ATOM | 200 | CB | GLN | 38 | 32.874 | 30.155 | 47.643 | 1.00 | 36.29 |
| ATOM | 201 | CG | GLN | 38 | 32.128 | 30.122 | 48.950 | 1.00 | 37.44 |
| ATOM | 202 | CD | GLN | 38 | 32.689 | 31.108 | 49.950 | 1.00 | 41.99 |
| ATOM | 203 | OE1 | GLN | 38 | 33.841 | 30.992 | 50.376 | 1.00 | 44.33 |
| ATOM | 204 | NE2 | GLN | 38 | 31.880 | 32.091 | 50.331 | 1.00 | 44.58 |
| ATOM | 205 | C | GLN | 38 | 33.184 | 29.155 | 45.382 | 1.00 | 35.21 |
| ATOM | 206 | O | GLN | 38 | 32.454 | 29.557 | 44.486 | 1.00 | 34.82 |
| ATOM | 207 | N | LYS | 39 | 34.467 | 28.884 | 45.188 | 1.00 | 36.41 |
| ATOM | 208 | CA | LYS | 39 | 35.069 | 29.081 | 43.875 | 1.00 | 38.60 |
| ATOM | 209 | CB | LYS | 39 | 36.560 | 28.708 | 43.888 | 1.00 | 42.47 |
| ATOM | 210 | CG | LYS | 39 | 37.395 | 29.263 | 42.714 | 1.00 | 45.02 |
| ATOM | 211 | CD | LYS | 39 | 37.638 | 30.775 | 42.861 | 1.00 | 49.54 |
| ATOM | 212 | CE | LYS | 39 | 38.523 | 31.365 | 41.752 | 1.00 | 51.65 |
| ATOM | 213 | NZ | LYS | 39 | 38.621 | 32.865 | 41.821 | 1.00 | 53.58 |
| ATOM | 214 | C | LYS | 39 | 34.339 | 28.196 | 42.884 | 1.00 | 38.31 |
| ATOM | 215 | O | LYS | 39 | 34.229 | 28.534 | 41.710 | 1.00 | 40.28 |
| ATOM | 216 | N | GLU | 40 | 33.827 | 27.066 | 43.369 | 1.00 | 37.21 |
| ATOM | 217 | CA | GLU | 40 | 33.117 | 26.107 | 42.525 | 1.00 | 34.69 |
| ATOM | 218 | CB | GLU | 40 | 33.329 | 24.705 | 43.072 | 1.00 | 32.80 |
| ATOM | 219 | CG | GLU | 40 | 34.742 | 24.245 | 42.900 | 1.00 | 33.53 |
| ATOM | 220 | CD | GLU | 40 | 35.164 | 24.348 | 41.459 | 1.00 | 36.48 |
| ATOM | 221 | OE1 | GLU | 40 | 34.318 | 24.044 | 40.589 | 1.00 | 39.36 |
| ATOM | 222 | OE2 | GLU | 40 | 36.326 | 24.720 | 41.187 | 1.00 | 37.18 |
| ATOM | 223 | C | GLU | 40 | 31.632 | 26.387 | 42.375 | 1.00 | 34.48 |
| ATOM | 224 | O | GLU | 40 | 31.040 | 26.110 | 41.332 | 1.00 | 32.30 |
| ATOM | 225 | N | MET | 41 | 31.030 | 26.928 | 43.425 | 1.00 | 35.61 |
| ATOM | 226 | CA | MET | 41 | 29.621 | 27.256 | 43.373 | 1.00 | 39.30 |
| ATOM | 227 | CB | MET | 41 | 29.155 | 27.852 | 44.692 | 1.00 | 39.16 |
| ATOM | 228 | CG | MET | 41 | 29.146 | 26.910 | 45.867 | 1.00 | 40.71 |
| ATOM | 229 | SD | MET | 41 | 27.930 | 27.569 | 47.040 | 1.00 | 46.34 |
| ATOM | 230 | CE | MET | 41 | 28.978 | 28.338 | 48.243 | 1.00 | 46.54 |
| ATOM | 231 | C | MET | 41 | 29.336 | 28.258 | 42.251 | 1.00 | 42.24 |
| ATOM | 232 | O | MET | 41 | 28.358 | 28.113 | 41.517 | 1.00 | 44.97 |
| ATOM | 233 | N | ASP | 42 | 30.173 | 29.284 | 42.118 | 1.00 | 43.47 |
| ATOM | 234 | CA | ASP | 42 | 29.952 | 30.274 | 41.069 | 1.00 | 42.69 |
| ATOM | 235 | CB | ASP | 42 | 30.848 | 31.497 | 41.249 | 1.00 | 44.70 |
| ATOM | 236 | CG | ASP | 42 | 30.548 | 32.254 | 42.523 | 1.00 | 49.63 |
| ATOM | 237 | OD1 | ASP | 42 | 31.352 | 32.128 | 43.477 | 1.00 | 52.14 |
| ATOM | 238 | OD2 | ASP | 42 | 29.510 | 32.968 | 42.572 | 1.00 | 49.66 |
| ATOM | 239 | C | ASP | 42 | 30.248 | 29.641 | 39.739 | 1.00 | 41.40 |
| ATOM | 240 | O | ASP | 42 | 29.550 | 29.880 | 38.759 | 1.00 | 41.06 |
| ATOM | 241 | N | ARG | 43 | 31.289 | 28.826 | 39.707 | 1.00 | 39.70 |
| ATOM | 242 | CA | ARG | 43 | 31.668 | 28.171 | 38.477 | 1.00 | 39.99 |
| ATOM | 243 | CB | ARG | 43 | 32.835 | 27.227 | 38.739 | 1.00 | 43.98 |
| ATOM | 244 | CG | ARG | 43 | 33.329 | 26.482 | 37.516 | 1.00 | 49.72 |
| ATOM | 245 | CD | ARG | 43 | 34.636 | 25.777 | 37.831 | 1.00 | 55.67 |
| ATOM | 246 | NE | ARG | 43 | 34.962 | 24.746 | 36.854 | 1.00 | 62.98 |
| ATOM | 247 | CZ | ARG | 43 | 36.062 | 24.002 | 36.899 | 1.00 | 67.95 |
| ATOM | 248 | NH1 | ARG | 43 | 36.950 | 24.178 | 37.877 | 1.00 | 69.41 |
| ATOM | 249 | NH2 | ARG | 43 | 36.269 | 23.075 | 35.969 | 1.00 | 70.32 |
| ATOM | 250 | C | ARG | 43 | 30.488 | 27.417 | 37.881 | 1.00 | 38.35 |
| ATOM | 251 | O | ARG | 43 | 30.253 | 27.493 | 36.677 | 1.00 | 38.07 |
| ATOM | 252 | N | GLY | 44 | 29.739 | 26.709 | 38.728 | 1.00 | 36.44 |
| ATOM | 253 | CA | GLY | 44 | 28.592 | 25.938 | 38.262 | 1.00 | 32.80 |
| ATOM | 254 | C | GLY | 44 | 27.344 | 26.772 | 38.062 | 1.00 | 31.71 |
| ATOM | 255 | O | GLY | 44 | 26.483 | 26.448 | 37.251 | 1.00 | 30.43 |
| ATOM | 256 | N | LEU | 45 | 27.258 | 27.854 | 38.820 | 1.00 | 31.23 |
| ATOM | 257 | CA | LEU | 45 | 26.144 | 28.774 | 38.761 | 1.00 | 31.72 |
| ATOM | 258 | CB | LEU | 45 | 26.168 | 29.638 | 40.010 | 1.00 | 30.96 |
| ATOM | 259 | CG | LEU | 45 | 25.063 | 29.363 | 41.013 | 1.00 | 34.38 |
| ATOM | 260 | CD1 | LEU | 45 | 25.346 | 30.066 | 42.334 | 1.00 | 34.74 |
| ATOM | 261 | CD2 | LEU | 45 | 23.750 | 29.849 | 40.413 | 1.00 | 37.12 |
| ATOM | 262 | C | LEU | 45 | 26.204 | 29.666 | 37.517 | 1.00 | 33.39 |
| ATOM | 263 | O | LEU | 45 | 25.184 | 30.211 | 37.086 | 1.00 | 34.01 |
| ATOM | 264 | N | ARG | 46 | 27.402 | 29.813 | 36.955 | 1.00 | 34.39 |
| ATOM | 265 | CA | ARG | 46 | 27.628 | 30.651 | 35.774 | 1.00 | 37.39 |
| ATOM | 266 | CB | ARG | 46 | 29.092 | 31.140 | 35.744 | 1.00 | 42.80 |
| ATOM | 267 | CG | ARG | 46 | 29.463 | 32.067 | 34.562 | 1.00 | 48.17 |
| ATOM | 268 | CD | ARG | 46 | 30.951 | 32.487 | 34.546 | 1.00 | 49.35 |
| ATOM | 269 | NE | ARG | 46 | 31.250 | 33.400 | 33.441 | 1.00 | 54.04 |
| ATOM | 270 | CZ | ARG | 46 | 30.599 | 34.542 | 33.216 | 1.00 | 57.98 |
| ATOM | 271 | NH1 | ARG | 46 | 29.608 | 34.915 | 34.019 | 1.00 | 56.34 |
| ATOM | 272 | NH2 | ARG | 46 | 30.936 | 35.316 | 32.187 | 1.00 | 59.91 |
| ATOM | 273 | C | ARG | 46 | 27.301 | 29.920 | 34.477 | 1.00 | 37.53 |
| ATOM | 274 | O | ARG | 46 | 27.773 | 28.804 | 34.243 | 1.00 | 38.11 |
| ATOM | 275 | N | LEU | 47 | 26.515 | 30.573 | 33.623 | 1.00 | 36.42 |
| ATOM | 276 | CA | LEU | 47 | 26.089 | 29.993 | 32.350 | 1.00 | 35.82 |
| ATOM | 277 | CB | LEU | 47 | 25.151 | 30.957 | 31.617 | 1.00 | 31.45 |
| ATOM | 278 | CG | LEU | 47 | 24.771 | 30.548 | 30.196 | 1.00 | 29.68 |
| ATOM | 279 | CD1 | LEU | 47 | 24.031 | 29.240 | 30.230 | 1.00 | 28.93 |
| ATOM | 280 | CD2 | LEU | 47 | 23.929 | 31.622 | 29.559 | 1.00 | 28.83 |
| ATOM | 281 | C | LEU | 47 | 27.223 | 29.578 | 31.418 | 1.00 | 37.14 |
| ATOM | 282 | O | LEU | 47 | 27.152 | 28.534 | 30.764 | 1.00 | 36.41 |
| ATOM | 283 | N | GLU | 48 | 28.272 | 30.383 | 31.347 | 1.00 | 39.28 |
| ATOM | 284 | CA | GLU | 48 | 29.371 | 30.034 | 30.462 | 1.00 | 42.38 |
| ATOM | 285 | CB | GLU | 48 | 30.448 | 31.126 | 30.473 | 1.00 | 43.91 |
| ATOM | 286 | CG | GLU | 48 | 30.126 | 32.354 | 29.631 | 1.00 | 46.02 |
| ATOM | 287 | CD | GLU | 48 | 29.022 | 33.215 | 30.221 | 1.00 | 48.71 |
| ATOM | 288 | OE1 | GLU | 48 | 28.581 | 34.157 | 29.524 | 1.00 | 48.10 |
| ATOM | 289 | OE2 | GLU | 48 | 28.600 | 32.959 | 31.375 | 1.00 | 49.31 |
| ATOM | 290 | C | GLU | 48 | 30.005 | 28.691 | 30.809 | 1.00 | 43.42 |
| ATOM | 291 | O | GLU | 48 | 30.593 | 28.045 | 29.939 | 1.00 | 43.61 |
| ATOM | 292 | N | THR | 49 | 29.873 | 28.262 | 32.066 | 1.00 | 44.28 |
| ATOM | 293 | CA | THR | 49 | 30.484 | 26.999 | 32.508 | 1.00 | 46.81 |
| ATOM | 294 | CB | THR | 49 | 31.761 | 27.267 | 33.366 | 1.00 | 47.70 |
| ATOM | 295 | OG1 | THR | 49 | 31.477 | 28.265 | 34.356 | 1.00 | 45.18 |
| ATOM | 296 | CG2 | THR | 49 | 32.921 | 27.739 | 32.486 | 1.00 | 48.17 |
| ATOM | 297 | C | THR | 49 | 29.595 | 26.024 | 33.293 | 1.00 | 46.50 |
| ATOM | 298 | O | THR | 49 | 30.043 | 24.932 | 33.683 | 1.00 | 45.72 |
| ATOM | 299 | N | HIS | 50 | 28.340 | 26.405 | 33.508 | 1.00 | 44.18 |
| ATOM | 300 | CA | HIS | 50 | 27.416 | 25.565 | 34.262 | 1.00 | 41.93 |
| ATOM | 301 | CB | HIS | 50 | 25.980 | 26.129 | 34.190 | 1.00 | 38.83 |
| ATOM | 302 | CG | HIS | 50 | 25.217 | 25.754 | 32.953 | 1.00 | 35.50 |
| ATOM | 303 | CD2 | HIS | 50 | 23.950 | 25.304 | 32.795 | 1.00 | 33.70 |
| ATOM | 304 | ND1 | HIS | 50 | 25.730 | 25.894 | 31.682 | 1.00 | 36.24 |
| ATOM | 305 | CE1 | HIS | 50 | 24.812 | 25.550 | 30.796 | 1.00 | 33.56 |
| ATOM | 306 | NE2 | HIS | 50 | 23.722 | 25.189 | 31.446 | 1.00 | 32.06 |
| ATOM | 307 | C | HIS | 50 | 27.447 | 24.117 | 33.804 | 1.00 | 41.73 |
| ATOM | 308 | O | HIS | 50 | 27.144 | 23.212 | 34.572 | 1.00 | 41.14 |
| ATOM | 309 | N | GLU | 51 | 27.848 | 23.883 | 32.566 | 1.00 | 42.00 |
| ATOM | 310 | CA | GLU | 51 | 27.863 | 22.519 | 32.103 | 1.00 | 45.79 |
| ATOM | 311 | CB | GLU | 51 | 27.573 | 22.463 | 30.617 | 1.00 | 46.76 |
| ATOM | 312 | CG | GLU | 51 | 27.523 | 21.048 | 30.100 | 1.00 | 50.98 |
| ATOM | 313 | CD | GLU | 51 | 26.521 | 20.885 | 28.989 | 1.00 | 53.94 |
| ATOM | 314 | OE1 | GLU | 51 | 25.313 | 21.082 | 29.253 | 1.00 | 55.61 |
| ATOM | 315 | OE2 | GLU | 51 | 26.940 | 20.560 | 27.857 | 1.00 | 55.48 |
| ATOM | 316 | C | GLU | 51 | 29.139 | 21.757 | 32.402 | 1.00 | 48.17 |
| ATOM | 317 | O | GLU | 51 | 29.094 | 20.657 | 32.953 | 1.00 | 49.35 |
| ATOM | 318 | N | GLU | 52 | 30.276 | 22.331 | 32.034 | 1.00 | 50.75 |
| ATOM | 319 | CA | GLU | 52 | 31.565 | 21.681 | 32.264 | 1.00 | 52.07 |
| ATOM | 320 | CB | GLU | 52 | 32.633 | 22.321 | 31.352 | 1.00 | 56.66 |
| ATOM | 321 | CG | GLU | 52 | 32.768 | 23.854 | 31.476 | 1.00 | 63.81 |
| ATOM | 322 | CD | GLU | 52 | 33.420 | 24.528 | 30.253 | 1.00 | 67.84 |
| ATOM | 323 | OE1 | GLU | 52 | 33.601 | 25.770 | 30.278 | 1.00 | 68.83 |
| ATOM | 324 | OE2 | GLU | 52 | 33.742 | 23.826 | 29.266 | 1.00 | 70.00 |
| ATOM | 325 | C | GLU | 52 | 31.982 | 21.760 | 33.738 | 1.00 | 49.95 |
| ATOM | 326 | O | GLU | 52 | 33.013 | 21.215 | 34.132 | 1.00 | 47.47 |
| ATOM | 327 | N | ALA | 53 | 31.162 | 22.429 | 34.548 | 1.00 | 48.46 |
| ATOM | 328 | CA | ALA | 53 | 31.449 | 22.594 | 35.972 | 1.00 | 47.88 |
| ATOM | 329 | CB | ALA | 53 | 30.418 | 23.510 | 36.615 | 1.00 | 47.30 |
| ATOM | 330 | C | ALA | 53 | 31.510 | 21.278 | 36.731 | 1.00 | 46.84 |
| ATOM | 331 | O | ALA | 53 | 31.287 | 20.206 | 36.172 | 1.00 | 48.51 |
| ATOM | 332 | N | SER | 54 | 31.816 | 21.353 | 38.016 | 1.00 | 44.67 |
| ATOM | 333 | CA | SER | 54 | 31.895 | 20.133 | 38.792 | 1.00 | 42.38 |
| ATOM | 334 | CB | SER | 54 | 33.201 | 20.090 | 39.581 | 1.00 | 44.26 |
| ATOM | 335 | OG | SER | 54 | 33.290 | 18.883 | 40.316 | 1.00 | 45.49 |
| ATOM | 336 | C | SER | 54 | 30.712 | 20.059 | 39.734 | 1.00 | 39.72 |
| ATOM | 337 | O | SER | 54 | 30.058 | 19.028 | 39.841 | 1.00 | 41.09 |
| ATOM | 338 | N | VAL | 55 | 30.440 | 21.165 | 40.411 | 1.00 | 34.77 |
| ATOM | 339 | CA | VAL | 55 | 29.326 | 21.239 | 41.343 | 1.00 | 30.58 |
| ATOM | 340 | CB | VAL | 55 | 29.682 | 22.186 | 42.498 | 1.00 | 28.73 |
| ATOM | 341 | CG1 | VAL | 55 | 28.480 | 22.433 | 43.383 | 1.00 | 30.75 |
| ATOM | 342 | CG2 | VAL | 55 | 30.814 | 21.596 | 43.297 | 1.00 | 25.80 |
| ATOM | 343 | C | VAL | 55 | 28.094 | 21.760 | 40.597 | 1.00 | 30.28 |
| ATOM | 344 | O | VAL | 55 | 27.704 | 22.920 | 40.745 | 1.00 | 32.16 |
| ATOM | 345 | N | LYS | 56 | 27.482 | 20.887 | 39.803 | 1.00 | 26.82 |
| ATOM | 346 | CA | LYS | 56 | 26.323 | 21.235 | 38.986 | 1.00 | 21.66 |
| ATOM | 347 | CB | LYS | 56 | 25.362 | 20.046 | 38.891 | 1.00 | 26.53 |
| ATOM | 348 | CG | LYS | 56 | 25.936 | 18.737 | 38.337 | 1.00 | 29.32 |
| ATOM | 349 | CD | LYS | 56 | 26.311 | 18.836 | 36.875 | 1.00 | 29.86 |
| ATOM | 350 | CE | LYS | 56 | 27.609 | 19.592 | 36.698 | 1.00 | 29.73 |
| ATOM | 351 | NZ | LYS | 56 | 27.932 | 19.759 | 35.259 | 1.00 | 32.80 |
| ATOM | 352 | C | LYS | 56 | 25.520 | 22.470 | 39.374 | 1.00 | 17.56 |
| ATOM | 353 | O | LYS | 56 | 25.133 | 23.236 | 38.498 | 1.00 | 15.95 |
| ATOM | 354 | N | MET | 57 | 25.257 | 22.660 | 40.665 | 1.00 | 14.30 |
| ATOM | 355 | CA | MET | 57 | 24.462 | 23.803 | 41.128 | 1.00 | 12.73 |
| ATOM | 356 | CB | MET | 57 | 25.277 | 25.089 | 41.059 | 1.00 | 9.92 |
| ATOM | 357 | CG | MET | 57 | 26.515 | 25.090 | 41.930 | 1.00 | 6.47 |
| ATOM | 358 | SD | MET | 57 | 26.219 | 25.164 | 43.694 | 1.00 | 8.00 |
| ATOM | 359 | CE | MET | 57 | 25.523 | 26.842 | 43.905 | 1.00 | 1.00 |
| ATOM | 360 | C | MET | 57 | 23.207 | 23.953 | 40.270 | 1.00 | 14.05 |
| ATOM | 361 | O | MET | 57 | 23.000 | 24.972 | 39.610 | 1.00 | 12.36 |
| ATOM | 362 | N | LEU | 58 | 22.371 | 22.923 | 40.290 | 1.00 | 17.80 |
| ATOM | 363 | CA | LEU | 58 | 21.154 | 22.914 | 39.498 | 1.00 | 19.02 |
| ATOM | 364 | CB | LEU | 58 | 20.710 | 21.466 | 39.245 | 1.00 | 18.03 |
| ATOM | 365 | CG | LEU | 58 | 21.726 | 20.444 | 38.720 | 1.00 | 16.28 |
| ATOM | 366 | CD1 | LEU | 58 | 21.193 | 19.068 | 39.021 | 1.00 | 20.44 |
| ATOM | 367 | CD2 | LEU | 58 | 21.999 | 20.608 | 37.233 | 1.00 | 15.03 |
| ATOM | 368 | C | LEU | 58 | 20.005 | 23.696 | 40.134 | 1.00 | 20.20 |
| ATOM | 369 | O | LEU | 58 | 19.752 | 23.602 | 41.340 | 1.00 | 19.91 |
| ATOM | 370 | N | PRO | 59 | 19.316 | 24.507 | 39.320 | 1.00 | 20.57 |
| ATOM | 371 | CD | PRO | 59 | 19.856 | 24.939 | 38.022 | 1.00 | 20.39 |
| ATOM | 372 | CA | PRO | 59 | 18.171 | 25.342 | 39.694 | 1.00 | 22.50 |
| ATOM | 373 | CB | PRO | 59 | 17.939 | 26.168 | 38.437 | 1.00 | 22.07 |
| ATOM | 374 | CG | PRO | 59 | 19.306 | 26.329 | 37.906 | 1.00 | 21.92 |
| ATOM | 375 | C | PRO | 59 | 16.975 | 24.437 | 40.010 | 1.00 | 23.49 |
| ATOM | 376 | O | PRO | 59 | 16.698 | 23.504 | 39.264 | 1.00 | 25.36 |
| ATOM | 377 | N | THR | 60 | 16.258 | 24.714 | 41.092 | 1.00 | 22.35 |
| ATOM | 378 | CA | THR | 60 | 15.133 | 23.871 | 41.469 | 1.00 | 20.99 |
| ATOM | 379 | CB | THR | 60 | 15.097 | 23.607 | 42.964 | 1.00 | 22.35 |
| ATOM | 380 | OG1 | THR | 60 | 14.823 | 24.837 | 43.647 | 1.00 | 24.53 |
| ATOM | 381 | CG2 | THR | 60 | 16.408 | 23.049 | 43.441 | 1.00 | 24.88 |
| ATOM | 382 | C | THR | 60 | 13.815 | 24.516 | 41.160 | 1.00 | 20.21 |
| ATOM | 383 | O | THR | 60 | 12.793 | 23.848 | 41.119 | 1.00 | 24.18 |
| ATOM | 384 | N | TYR | 61 | 13.839 | 25.822 | 40.973 | 1.00 | 19.09 |
| ATOM | 385 | CA | TYR | 61 | 12.628 | 26.595 | 40.715 | 1.00 | 20.03 |
| ATOM | 386 | CB | TYR | 61 | 11.955 | 26.172 | 39.427 | 1.00 | 13.50 |
| ATOM | 387 | CG | TYR | 61 | 12.581 | 26.830 | 38.234 | 1.00 | 13.18 |
| ATOM | 388 | CD1 | TYR | 61 | 12.028 | 27.983 | 37.666 | 1.00 | 8.00 |
| ATOM | 389 | CE1 | TYR | 61 | 12.596 | 28.551 | 36.536 | 1.00 | 4.24 |
| ATOM | 390 | CD2 | TYR | 61 | 13.725 | 26.281 | 37.647 | 1.00 | 14.04 |
| ATOM | 391 | CE2 | TYR | 61 | 14.296 | 26.843 | 36.529 | 1.00 | 10.05 |
| ATOM | 392 | CZ | TYR | 61 | 13.730 | 27.963 | 35.976 | 1.00 | 5.80 |
| ATOM | 393 | OH | TYR | 61 | 14.307 | 28.423 | 34.828 | 1.00 | 4.54 |
| ATOM | 394 | C | TYR | 61 | 11.620 | 26.572 | 41.833 | 1.00 | 21.95 |
| ATOM | 395 | O | TYR | 61 | 10.437 | 26.816 | 41.609 | 1.00 | 22.47 |
| ATOM | 396 | N | VAL | 62 | 12.102 | 26.293 | 43.037 | 1.00 | 24.47 |
| ATOM | 397 | CA | VAL | 62 | 11.265 | 26.288 | 44.218 | 1.00 | 29.86 |
| ATOM | 398 | CB | VAL | 62 | 11.750 | 25.231 | 45.207 | 1.00 | 28.92 |
| ATOM | 399 | CG1 | VAL | 62 | 10.780 | 25.091 | 46.370 | 1.00 | 28.30 |
| ATOM | 400 | CG2 | VAL | 62 | 11.909 | 23.926 | 44.480 | 1.00 | 28.58 |
| ATOM | 401 | C | VAL | 62 | 11.494 | 27.680 | 44.786 | 1.00 | 34.67 |
| ATOM | 402 | O | VAL | 62 | 11.584 | 27.879 | 45.993 | 1.00 | 39.01 |
| ATOM | 403 | N | ARG | 63 | 11.589 | 28.638 | 43.874 | 1.00 | 38.40 |
| ATOM | 404 | CA | ARG | 63 | 11.847 | 30.038 | 44.182 | 1.00 | 41.10 |
| ATOM | 405 | CB | ARG | 63 | 12.041 | 30.804 | 42.874 | 1.00 | 42.02 |
| ATOM | 406 | CG | ARG | 63 | 10.794 | 30.798 | 41.996 | 1.00 | 44.76 |
| ATOM | 407 | CD | ARG | 63 | 11.072 | 31.197 | 40.550 | 1.00 | 46.61 |
| ATOM | 408 | NE | ARG | 63 | 9.827 | 31.366 | 39.804 | 1.00 | 48.56 |
| ATOM | 409 | CZ | ARG | 63 | 8.972 | 30.381 | 39.541 | 1.00 | 50.39 |
| ATOM | 410 | NH1 | ARG | 63 | 9.225 | 29.145 | 39.955 | 1.00 | 50.83 |
| ATOM | 411 | NH2 | ARG | 63 | 7.854 | 30.635 | 38.875 | 1.00 | 51.11 |
| ATOM | 412 | C | ARG | 63 | 10.788 | 30.751 | 45.004 | 1.00 | 42.71 |
| ATOM | 413 | O | ARG | 63 | 9.790 | 30.167 | 45.424 | 1.00 | 41.58 |
| ATOM | 414 | N | SER | 64 | 11.047 | 32.036 | 45.224 | 1.00 | 46.12 |
| ATOM | 415 | CA | SER | 64 | 10.155 | 32.922 | 45.954 | 1.00 | 49.96 |
| ATOM | 416 | CB | SER | 64 | 10.400 | 32.826 | 47.454 | 1.00 | 50.57 |
| ATOM | 417 | OG | SER | 64 | 9.374 | 33.507 | 48.157 | 1.00 | 53.70 |
| ATOM | 418 | C | SER | 64 | 10.435 | 34.340 | 45.458 | 1.00 | 51.04 |
| ATOM | 419 | O | SER | 64 | 11.300 | 35.047 | 45.985 | 1.00 | 50.38 |
| ATOM | 420 | N | THR | 65 | 9.690 | 34.728 | 44.425 | 1.00 | 53.23 |
| ATOM | 421 | CA | THR | 65 | 9.827 | 36.031 | 43.791 | 1.00 | 54.89 |
| ATOM | 422 | CB | THR | 65 | 10.151 | 35.871 | 42.281 | 1.00 | 56.21 |
| ATOM | 423 | OG1 | THR | 65 | 9.094 | 35.158 | 41.622 | 1.00 | 55.23 |
| ATOM | 424 | CG2 | THR | 65 | 11.461 | 35.112 | 42.103 | 1.00 | 56.71 |
| ATOM | 425 | C | THR | 65 | 8.582 | 36.911 | 43.939 | 1.00 | 56.01 |
| ATOM | 426 | O | THR | 65 | 7.503 | 36.430 | 44.291 | 1.00 | 56.26 |
| ATOM | 427 | N | PRO | 66 | 8.728 | 38.222 | 43.676 | 1.00 | 56.49 |
| ATOM | 428 | CD | PRO | 66 | 10.019 | 38.866 | 43.377 | 1.00 | 56.96 |
| ATOM | 429 | CA | PRO | 66 | 7.666 | 39.228 | 43.758 | 1.00 | 56.28 |
| ATOM | 430 | CB | PRO | 66 | 8.369 | 40.502 | 43.313 | 1.00 | 57.08 |
| ATOM | 431 | CG | PRO | 66 | 9.759 | 40.287 | 43.786 | 1.00 | 58.08 |
| ATOM | 432 | C | PRO | 66 | 6.487 | 38.901 | 42.864 | 1.00 | 56.75 |
| ATOM | 433 | O | PRO | 66 | 5.477 | 39.604 | 42.874 | 1.00 | 57.23 |
| ATOM | 434 | N | GLU | 67 | 6.631 | 37.849 | 42.072 | 1.00 | 56.42 |
| ATOM | 435 | CA | GLU | 67 | 5.540 | 37.445 | 41.193 | 1.00 | 56.82 |
| ATOM | 436 | CB | GLU | 67 | 6.048 | 36.487 | 40.115 | 1.00 | 61.19 |
| ATOM | 437 | CG | GLU | 67 | 6.421 | 35.108 | 40.637 | 1.00 | 66.99 |
| ATOM | 438 | CD | GLU | 67 | 7.123 | 34.261 | 39.594 | 1.00 | 69.61 |
| ATOM | 439 | OE1 | GLU | 67 | 8.253 | 34.618 | 39.201 | 1.00 | 70.19 |
| ATOM | 440 | OE2 | GLU | 67 | 6.541 | 33.241 | 39.168 | 1.00 | 70.18 |
| ATOM | 441 | C | GLU | 67 | 4.406 | 36.803 | 41.984 | 1.00 | 54.30 |
| ATOM | 442 | O | GLU | 67 | 3.241 | 36.940 | 41.633 | 1.00 | 54.25 |
| ATOM | 443 | N | GLY | 68 | 4.753 | 36.116 | 43.076 | 1.00 | 50.50 |
| ATOM | 444 | CA | GLY | 68 | 3.741 | 35.478 | 43.901 | 1.00 | 45.77 |
| ATOM | 445 | C | GLY | 68 | 4.166 | 34.087 | 44.316 | 1.00 | 43.04 |
| ATOM | 446 | O | GLY | 68 | 3.626 | 33.503 | 45.259 | 1.00 | 40.69 |
| ATOM | 447 | N | SER | 69 | 5.154 | 33.564 | 43.599 | 1.00 | 42.30 |
| ATOM | 448 | CA | SER | 69 | 5.690 | 32.230 | 43.845 | 1.00 | 41.02 |
| ATOM | 449 | CB | SER | 69 | 6.769 | 31.902 | 42.804 | 1.00 | 41.03 |
| ATOM | 450 | OG | SER | 69 | 6.438 | 32.404 | 41.517 | 1.00 | 42.34 |
| ATOM | 451 | C | SER | 69 | 6.301 | 32.126 | 45.240 | 1.00 | 39.68 |
| ATOM | 452 | O | SER | 69 | 7.163 | 32.920 | 45.607 | 1.00 | 38.89 |
| ATOM | 453 | N | GLU | 70 | 5.857 | 31.143 | 46.014 | 1.00 | 39.96 |
| ATOM | 454 | CA | GLU | 70 | 6.388 | 30.942 | 47.355 | 1.00 | 40.53 |
| ATOM | 455 | CB | GLU | 70 | 5.265 | 31.074 | 48.391 | 1.00 | 44.80 |
| ATOM | 456 | CG | GLU | 70 | 4.675 | 32.483 | 48.492 | 1.00 | 52.74 |
| ATOM | 457 | CD | GLU | 70 | 5.705 | 33.554 | 48.900 | 1.00 | 58.55 |
| ATOM | 458 | OE1 | GLU | 70 | 5.362 | 34.763 | 48.866 | 1.00 | 59.55 |
| ATOM | 459 | OE2 | GLU | 70 | 6.852 | 33.192 | 49.258 | 1.00 | 60.30 |
| ATOM | 460 | C | GLU | 70 | 7.075 | 29.583 | 47.483 | 1.00 | 38.65 |
| ATOM | 461 | O | GLU | 70 | 6.807 | 28.660 | 46.704 | 1.00 | 37.89 |
| ATOM | 462 | N | VAL | 71 | 7.962 | 29.459 | 48.466 | 1.00 | 35.96 |
| ATOM | 463 | CA | VAL | 71 | 8.670 | 28.207 | 48.653 | 1.00 | 34.46 |
| ATOM | 464 | CB | VAL | 71 | 9.723 | 28.319 | 49.755 | 1.00 | 33.00 |
| ATOM | 465 | CG1 | VAL | 71 | 10.236 | 26.949 | 50.120 | 1.00 | 33.91 |
| ATOM | 466 | CG2 | VAL | 71 | 10.885 | 29.152 | 49.249 | 1.00 | 32.56 |
| ATOM | 467 | C | VAL | 71 | 7.730 | 27.042 | 48.931 | 1.00 | 34.75 |
| ATOM | 468 | O | VAL | 71 | 7.851 | 25.985 | 48.310 | 1.00 | 37.23 |
| ATOM | 469 | N | GLY | 72 | 6.783 | 27.219 | 49.841 | 1.00 | 33.37 |
| ATOM | 470 | CA | GLY | 72 | 5.842 | 26.139 | 50.105 | 1.00 | 32.39 |
| ATOM | 471 | C | GLY | 72 | 5.066 | 25.644 | 48.879 | 1.00 | 31.10 |
| ATOM | 472 | O | GLY | 72 | 4.631 | 24.493 | 48.859 | 1.00 | 28.98 |
| ATOM | 473 | N | ASP | 73 | 4.878 | 26.503 | 47.870 | 1.00 | 31.05 |
| ATOM | 474 | CA | ASP | 73 | 4.156 | 26.129 | 46.650 | 1.00 | 31.14 |
| ATOM | 475 | CB | ASP | 73 | 4.389 | 27.147 | 45.532 | 1.00 | 34.00 |
| ATOM | 476 | CG | ASP | 73 | 3.759 | 28.491 | 45.817 | 1.00 | 38.43 |
| ATOM | 477 | OD1 | ASP | 73 | 3.758 | 29.355 | 44.907 | 1.00 | 41.88 |
| ATOM | 478 | OD2 | ASP | 73 | 3.262 | 28.690 | 46.945 | 1.00 | 41.23 |
| ATOM | 479 | C | ASP | 73 | 4.675 | 24.785 | 46.189 | 1.00 | 30.89 |
| ATOM | 480 | O | ASP | 73 | 5.875 | 24.544 | 46.256 | 1.00 | 32.81 |
| ATOM | 481 | N | PHE | 74 | 3.796 | 23.921 | 45.694 | 1.00 | 28.84 |
| ATOM | 482 | CA | PHE | 74 | 4.233 | 22.595 | 45.271 | 1.00 | 27.21 |
| ATOM | 483 | CB | PHE | 74 | 4.728 | 21.834 | 46.502 | 1.00 | 26.13 |
| ATOM | 484 | CG | PHE | 74 | 5.407 | 20.551 | 46.185 | 1.00 | 25.61 |
| ATOM | 485 | CD1 | PHE | 74 | 6.641 | 20.546 | 45.547 | 1.00 | 29.29 |
| ATOM | 486 | CD2 | PHE | 74 | 4.805 | 19.344 | 46.496 | 1.00 | 24.94 |
| ATOM | 487 | CE1 | PHE | 74 | 7.259 | 19.354 | 45.213 | 1.00 | 31.36 |
| ATOM | 488 | CE2 | PHE | 74 | 5.408 | 18.149 | 46.168 | 1.00 | 27.38 |
| ATOM | 489 | CZ | PHE | 74 | 6.640 | 18.149 | 45.527 | 1.00 | 30.18 |
| ATOM | 490 | C | PHE | 74 | 3.080 | 21.837 | 44.604 | 1.00 | 27.31 |
| ATOM | 491 | O | PHE | 74 | 1.912 | 22.034 | 44.951 | 1.00 | 28.04 |
| ATOM | 492 | N | LEU | 75 | 3.402 | 20.965 | 43.654 | 1.00 | 23.99 |
| ATOM | 493 | CA | LEU | 75 | 2.370 | 20.214 | 42.958 | 1.00 | 20.00 |
| ATOM | 494 | CB | LEU | 75 | 2.222 | 20.725 | 41.534 | 1.00 | 19.88 |
| ATOM | 495 | CG | LEU | 75 | 0.868 | 20.487 | 40.865 | 1.00 | 21.27 |
| ATOM | 496 | CD1 | LEU | 75 | 1.083 | 20.282 | 39.354 | 1.00 | 19.58 |
| ATOM | 497 | CD2 | LEU | 75 | 0.190 | 19.279 | 41.474 | 1.00 | 18.85 |
| ATOM | 498 | C | LEU | 75 | 2.755 | 18.758 | 42.911 | 1.00 | 18.82 |
| ATOM | 499 | O | LEU | 75 | 3.587 | 18.369 | 42.102 | 1.00 | 19.49 |
| ATOM | 500 | N | SER | 76 | 2.143 | 17.957 | 43.774 | 1.00 | 21.08 |
| ATOM | 501 | CA | SER | 76 | 2.434 | 16.530 | 43.834 | 1.00 | 22.49 |
| ATOM | 502 | CB | SER | 76 | 2.333 | 16.001 | 45.261 | 1.00 | 22.74 |
| ATOM | 503 | OG | SER | 76 | 2.591 | 14.612 | 45.292 | 1.00 | 20.37 |
| ATOM | 504 | C | SER | 76 | 1.507 | 15.720 | 42.967 | 1.00 | 23.58 |
| ATOM | 505 | O | SER | 76 | 0.309 | 15.980 | 42.866 | 1.00 | 23.06 |
| ATOM | 506 | N | LEU | 77 | 2.064 | 14.686 | 42.378 | 1.00 | 25.35 |
| ATOM | 507 | CA | LEU | 77 | 1.280 | 13.862 | 41.509 | 1.00 | 27.55 |
| ATOM | 508 | CB | LEU | 77 | 1.758 | 14.122 | 40.089 | 1.00 | 29.38 |
| ATOM | 509 | CG | LEU | 77 | 1.176 | 13.275 | 38.980 | 1.00 | 32.75 |
| ATOM | 510 | CD1 | LEU | 77 | −0.334 | 13.434 | 38.974 | 1.00 | 34.55 |
| ATOM | 511 | CD2 | LEU | 77 | 1.796 | 13.695 | 37.661 | 1.00 | 32.83 |
| ATOM | 512 | C | LEU | 77 | 1.445 | 12.402 | 41.913 | 1.00 | 28.86 |
| ATOM | 513 | O | LEU | 77 | 2.527 | 11.826 | 41.760 | 1.00 | 26.84 |
| ATOM | 514 | N | ASP | 78 | 0.386 | 11.811 | 42.465 | 1.00 | 29.41 |
| ATOM | 515 | CA | ASP | 78 | 0.457 | 10.407 | 42.865 | 1.00 | 30.41 |
| ATOM | 516 | CB | ASP | 78 | −0.150 | 10.186 | 44.255 | 1.00 | 31.87 |
| ATOM | 517 | CG | ASP | 78 | −0.286 | 8.702 | 44.606 | 1.00 | 33.99 |
| ATOM | 518 | OD1 | ASP | 78 | −1.025 | 7.993 | 43.894 | 1.00 | 35.38 |
| ATOM | 519 | OD2 | ASP | 78 | 0.338 | 8.241 | 45.586 | 1.00 | 33.31 |
| ATOM | 520 | C | ASP | 78 | −0.270 | 9.530 | 41.860 | 1.00 | 29.41 |
| ATOM | 521 | O | ASP | 78 | −1.484 | 9.587 | 41.732 | 1.00 | 29.74 |
| ATOM | 522 | N | LEU | 79 | 0.472 | 8.710 | 41.143 | 1.00 | 27.93 |
| ATOM | 523 | CA | LEU | 79 | −0.169 | 7.858 | 40.184 | 1.00 | 28.08 |
| ATOM | 524 | CB | LEU | 79 | 0.323 | 8.173 | 38.781 | 1.00 | 25.78 |
| ATOM | 525 | CG | LEU | 79 | 1.676 | 7.627 | 38.371 | 1.00 | 24.57 |
| ATOM | 526 | CD1 | LEU | 79 | 1.845 | 7.871 | 36.904 | 1.00 | 25.82 |
| ATOM | 527 | CD2 | LEU | 79 | 2.779 | 8.274 | 39.166 | 1.00 | 26.37 |
| ATOM | 528 | C | LEU | 79 | 0.114 | 6.420 | 40.548 | 1.00 | 31.25 |
| ATOM | 529 | O | LEU | 79 | 1.265 | 6.017 | 40.712 | 1.00 | 32.14 |
| ATOM | 530 | N | GLY | 80 | −0.955 | 5.652 | 40.699 | 1.00 | 34.99 |
| ATOM | 531 | CA | GLY | 80 | −0.812 | 4.259 | 41.056 | 1.00 | 38.29 |
| ATOM | 532 | C | GLY | 80 | −2.088 | 3.499 | 40.776 | 1.00 | 40.81 |
| ATOM | 533 | O | GLY | 80 | −3.100 | 3.686 | 41.452 | 1.00 | 40.77 |
| ATOM | 534 | N | GLY | 81 | −2.038 | 2.642 | 39.765 | 1.00 | 43.19 |
| ATOM | 535 | CA | GLY | 81 | −3.197 | 1.850 | 39.422 | 1.00 | 45.84 |
| ATOM | 536 | C | GLY | 81 | −3.936 | 2.428 | 38.244 | 1.00 | 49.22 |
| ATOM | 537 | O | GLY | 81 | −3.328 | 2.825 | 37.241 | 1.00 | 49.20 |
| ATOM | 538 | N | THR | 82 | −5.260 | 2.465 | 38.365 | 1.00 | 51.93 |
| ATOM | 539 | CA | THR | 82 | −6.117 | 3.003 | 37.312 | 1.00 | 54.41 |
| ATOM | 540 | CB | THR | 82 | −7.344 | 2.090 | 37.060 | 1.00 | 56.74 |
| ATOM | 541 | OG1 | THR | 82 | −6.908 | 0.727 | 36.952 | 1.00 | 60.43 |
| ATOM | 542 | CG2 | THR | 82 | −8.043 | 2.473 | 35.752 | 1.00 | 58.23 |
| ATOM | 543 | C | THR | 82 | −6.584 | 4.382 | 37.759 | 1.00 | 52.48 |
| ATOM | 544 | O | THR | 82 | −7.308 | 5.077 | 37.046 | 1.00 | 52.21 |
| ATOM | 545 | N | ASN | 83 | −6.148 | 4.778 | 38.946 | 1.00 | 50.63 |
| ATOM | 546 | CA | ASN | 83 | −6.523 | 6.071 | 39.466 | 1.00 | 50.52 |
| ATOM | 547 | CB | ASN | 83 | −7.574 | 5.911 | 40.568 | 1.00 | 53.97 |
| ATOM | 548 | CG | ASN | 83 | −8.955 | 5.560 | 40.020 | 1.00 | 58.88 |
| ATOM | 549 | OD1 | ASN | 83 | −9.508 | 6.290 | 39.190 | 1.00 | 60.51 |
| ATOM | 550 | ND2 | ASN | 83 | −9.521 | 4.444 | 40.489 | 1.00 | 60.30 |
| ATOM | 551 | C | ASN | 83 | −5.338 | 6.861 | 39.997 | 1.00 | 48.79 |
| ATOM | 552 | O | ASN | 83 | −4.682 | 6.442 | 40.956 | 1.00 | 48.09 |
| ATOM | 553 | N | PHE | 84 | −5.068 | 8.003 | 39.356 | 1.00 | 45.51 |
| ATOM | 554 | CA | PHE | 84 | −3.995 | 8.907 | 39.772 | 1.00 | 40.32 |
| ATOM | 555 | CB | PHE | 84 | −2.998 | 9.145 | 38.644 | 1.00 | 39.20 |
| ATOM | 556 | CG | PHE | 84 | −3.436 | 10.175 | 37.652 | 1.00 | 39.52 |
| ATOM | 557 | CD1 | PHE | 84 | −4.096 | 9.802 | 36.494 | 1.00 | 40.87 |
| ATOM | 558 | CD2 | PHE | 84 | −3.159 | 11.524 | 37.860 | 1.00 | 39.69 |
| ATOM | 559 | CE1 | PHE | 84 | −4.479 | 10.758 | 35.549 | 1.00 | 41.79 |
| ATOM | 560 | CE2 | PHE | 84 | −3.540 | 12.490 | 36.922 | 1.00 | 40.16 |
| ATOM | 561 | CZ | PHE | 84 | −4.198 | 12.105 | 35.762 | 1.00 | 40.38 |
| ATOM | 562 | C | PHE | 84 | −4.604 | 10.246 | 40.176 | 1.00 | 37.84 |
| ATOM | 563 | O | PHE | 84 | −5.405 | 10.806 | 39.439 | 1.00 | 37.11 |
| ATOM | 564 | N | ARG | 85 | −4.216 | 10.762 | 41.338 | 1.00 | 36.37 |
| ATOM | 565 | CA | ARG | 85 | −4.738 | 12.032 | 41.840 | 1.00 | 35.14 |
| ATOM | 566 | CB | ARG | 85 | −5.496 | 11.779 | 43.136 | 1.00 | 39.80 |
| ATOM | 567 | CG | ARG | 85 | −4.888 | 10.677 | 43.970 | 1.00 | 47.71 |
| ATOM | 568 | CD | ARG | 85 | −5.948 | 9.964 | 44.805 | 1.00 | 55.73 |
| ATOM | 569 | NE | ARG | 85 | −5.391 | 8.801 | 45.493 | 1.00 | 62.76 |
| ATOM | 570 | CZ | ARG | 85 | −4.799 | 7.772 | 44.883 | 1.00 | 65.65 |
| ATOM | 571 | NH1 | ARG | 85 | −4.684 | 7.749 | 43.557 | 1.00 | 63.79 |
| ATOM | 572 | NH2 | ARG | 85 | −4.314 | 6.765 | 45.605 | 1.00 | 66.67 |
| ATOM | 573 | C | ARG | 85 | −3.664 | 13.088 | 42.075 | 1.00 | 32.14 |
| ATOM | 574 | O | ARG | 85 | −2.561 | 12.772 | 42.522 | 1.00 | 32.77 |
| ATOM | 575 | N | VAL | 86 | −3.977 | 14.345 | 41.778 | 1.00 | 27.45 |
| ATOM | 576 | CA | VAL | 86 | −2.997 | 15.405 | 41.983 | 1.00 | 26.49 |
| ATOM | 577 | CB | VAL | 86 | −2.975 | 16.400 | 40.821 | 1.00 | 24.77 |
| ATOM | 578 | CG1 | VAL | 86 | −3.033 | 15.655 | 39.510 | 1.00 | 26.70 |
| ATOM | 579 | CG2 | VAL | 86 | −4.109 | 17.373 | 40.948 | 1.00 | 24.73 |
| ATOM | 580 | C | VAL | 86 | −3.292 | 16.177 | 43.257 | 1.00 | 26.66 |
| ATOM | 581 | O | VAL | 86 | −4.401 | 16.121 | 43.779 | 1.00 | 28.06 |
| ATOM | 582 | N | MET | 87 | −2.289 | 16.888 | 43.757 | 1.00 | 26.93 |
| ATOM | 583 | CA | MET | 87 | −2.427 | 17.677 | 44.973 | 1.00 | 25.08 |
| ATOM | 584 | CB | MET | 87 | −1.748 | 16.979 | 46.138 | 1.00 | 25.05 |
| ATOM | 585 | CG | MET | 87 | −1.674 | 17.833 | 47.375 | 1.00 | 24.83 |
| ATOM | 586 | SD | MET | 87 | −0.509 | 17.090 | 48.503 | 1.00 | 30.68 |
| ATOM | 587 | CE | MET | 87 | −1.544 | 16.749 | 49.894 | 1.00 | 29.41 |
| ATOM | 588 | C | MET | 87 | −1.768 | 19.021 | 44.774 | 1.00 | 24.52 |
| ATOM | 589 | O | MET | 87 | −0.638 | 19.097 | 44.298 | 1.00 | 27.12 |
| ATOM | 590 | N | LEU | 88 | −2.455 | 20.087 | 45.146 | 1.00 | 22.16 |
| ATOM | 591 | CA | LEU | 88 | −1.872 | 21.398 | 44.975 | 1.00 | 20.70 |
| ATOM | 592 | CB | LEU | 88 | −2.825 | 22.309 | 44.230 | 1.00 | 20.34 |
| ATOM | 593 | CG | LEU | 88 | −2.178 | 23.663 | 43.991 | 1.00 | 23.49 |
| ATOM | 594 | CD1 | LEU | 88 | −0.806 | 23.470 | 43.354 | 1.00 | 24.39 |
| ATOM | 595 | CD2 | LEU | 88 | −3.078 | 24.493 | 43.094 | 1.00 | 25.91 |
| ATOM | 596 | C | LEU | 88 | −1.535 | 22.021 | 46.301 | 1.00 | 19.94 |
| ATOM | 597 | O | LEU | 88 | −2.225 | 21.794 | 47.282 | 1.00 | 21.18 |
| ATOM | 598 | N | VAL | 89 | −0.463 | 22.799 | 46.343 | 1.00 | 20.16 |
| ATOM | 599 | CA | VAL | 89 | −0.082 | 23.462 | 47.580 | 1.00 | 21.15 |
| ATOM | 600 | CB | VAL | 89 | 0.984 | 22.676 | 48.357 | 1.00 | 14.95 |
| ATOM | 601 | CG1 | VAL | 89 | 1.292 | 23.385 | 49.657 | 1.00 | 7.73 |
| ATOM | 602 | CG2 | VAL | 89 | 0.515 | 21.268 | 48.609 | 1.00 | 10.59 |
| ATOM | 603 | C | VAL | 89 | 0.491 | 24.829 | 47.254 | 1.00 | 27.10 |
| ATOM | 604 | O | VAL | 89 | 1.410 | 24.939 | 46.442 | 1.00 | 27.22 |
| ATOM | 605 | N | LYS | 90 | −0.066 | 25.866 | 47.875 | 1.00 | 33.21 |
| ATOM | 606 | CA | LYS | 90 | 0.401 | 27.235 | 47.671 | 1.00 | 40.01 |
| ATOM | 607 | CB | LYS | 90 | −0.443 | 27.962 | 46.604 | 1.00 | 41.03 |
| ATOM | 608 | CG | LYS | 90 | −1.941 | 27.979 | 46.850 | 1.00 | 47.19 |
| ATOM | 609 | CD | LYS | 90 | −2.749 | 28.454 | 45.622 | 1.00 | 52.33 |
| ATOM | 610 | CE | LYS | 90 | −4.274 | 28.393 | 45.899 | 1.00 | 55.73 |
| ATOM | 611 | NZ | LYS | 90 | −5.161 | 28.724 | 44.731 | 1.00 | 56.02 |
| ATOM | 612 | C | LYS | 90 | 0.384 | 28.009 | 48.981 | 1.00 | 43.61 |
| ATOM | 613 | O | LYS | 90 | −0.577 | 27.943 | 49.747 | 1.00 | 44.04 |
| ATOM | 614 | N | VAL | 91 | 1.469 | 28.728 | 49.241 | 1.00 | 47.88 |
| ATOM | 615 | CA | VAL | 91 | 1.587 | 29.513 | 50.458 | 1.00 | 51.82 |
| ATOM | 616 | CB | VAL | 91 | 3.059 | 29.780 | 50.788 | 1.00 | 51.29 |
| ATOM | 617 | CG1 | VAL | 91 | 3.160 | 30.748 | 51.947 | 1.00 | 54.88 |
| ATOM | 618 | CG2 | VAL | 91 | 3.749 | 28.479 | 51.137 | 1.00 | 48.18 |
| ATOM | 619 | C | VAL | 91 | 0.849 | 30.846 | 50.355 | 1.00 | 55.01 |
| ATOM | 620 | O | VAL | 91 | 0.994 | 31.569 | 49.369 | 1.00 | 54.57 |
| ATOM | 621 | N | GLY | 92 | 0.060 | 31.157 | 51.382 | 1.00 | 59.16 |
| ATOM | 622 | CA | GLY | 92 | −0.696 | 32.396 | 51.401 | 1.00 | 64.58 |
| ATOM | 623 | C | GLY | 92 | −0.305 | 33.297 | 52.558 | 1.00 | 68.39 |
| ATOM | 624 | O | GLY | 92 | 0.637 | 32.992 | 53.295 | 1.00 | 66.92 |
| ATOM | 625 | N | GLU | 93 | −1.025 | 34.410 | 52.712 | 1.00 | 73.13 |
| ATOM | 626 | CA | GLU | 93 | −0.751 | 35.351 | 53.792 | 1.00 | 78.27 |
| ATOM | 627 | CB | GLU | 93 | −0.623 | 36.780 | 53.248 | 1.00 | 79.11 |
| ATOM | 628 | CG | GLU | 93 | 0.334 | 37.635 | 54.077 | 1.00 | 82.44 |
| ATOM | 629 | CD | GLU | 93 | 0.218 | 39.120 | 53.795 | 1.00 | 84.34 |
| ATOM | 630 | OE1 | GLU | 93 | −0.877 | 39.688 | 54.018 | 1.00 | 84.71 |
| ATOM | 631 | OE2 | GLU | 93 | 1.228 | 39.718 | 53.359 | 1.00 | 85.45 |
| ATOM | 632 | C | GLU | 93 | −1.813 | 35.309 | 54.904 | 1.00 | 80.72 |
| ATOM | 633 | O | GLU | 93 | −1.469 | 35.340 | 56.086 | 1.00 | 81.42 |
| ATOM | 634 | N | GLY | 94 | −3.093 | 35.240 | 54.536 | 1.00 | 83.03 |
| ATOM | 635 | CA | GLY | 94 | −4.153 | 35.182 | 55.538 | 1.00 | 85.37 |
| ATOM | 636 | C | GLY | 94 | −4.867 | 36.502 | 55.792 | 1.00 | 87.51 |
| ATOM | 637 | O | GLY | 94 | −4.356 | 37.562 | 55.430 | 1.00 | 88.65 |
| ATOM | 638 | N | GLU | 95 | −6.041 | 36.447 | 56.427 | 1.00 | 88.43 |
| ATOM | 639 | CA | GLU | 95 | −6.831 | 37.653 | 56.716 | 1.00 | 88.66 |
| ATOM | 640 | CB | GLU | 95 | −8.192 | 37.281 | 57.328 | 1.00 | 89.61 |
| ATOM | 641 | CG | GLU | 95 | −9.077 | 36.406 | 56.448 | 1.00 | 90.41 |
| ATOM | 642 | CD | GLU | 95 | −8.620 | 34.958 | 56.408 | 1.00 | 91.01 |
| ATOM | 643 | OE1 | GLU | 95 | −9.089 | 34.211 | 55.523 | 1.00 | 90.26 |
| ATOM | 644 | OE2 | GLU | 95 | −7.800 | 34.565 | 57.266 | 1.00 | 91.81 |
| ATOM | 645 | C | GLU | 95 | −6.115 | 38.625 | 57.652 | 1.00 | 88.62 |
| ATOM | 646 | O | GLU | 95 | −6.576 | 39.748 | 57.868 | 1.00 | 88.29 |
| ATOM | 647 | N | GLU | 96 | −4.991 | 38.182 | 58.208 | 1.00 | 89.03 |
| ATOM | 648 | CA | GLU | 96 | −4.200 | 38.995 | 59.124 | 1.00 | 88.80 |
| ATOM | 649 | CB | GLU | 96 | −4.065 | 38.282 | 60.476 | 1.00 | 88.55 |
| ATOM | 650 | CG | GLU | 96 | −5.368 | 38.155 | 61.268 | 1.00 | 89.59 |
| ATOM | 651 | CD | GLU | 96 | −6.400 | 37.262 | 60.593 | 1.00 | 90.56 |
| ATOM | 652 | OE1 | GLU | 96 | −6.163 | 36.040 | 60.481 | 1.00 | 90.53 |
| ATOM | 653 | OE2 | GLU | 96 | −7.452 | 37.785 | 60.172 | 1.00 | 90.67 |
| ATOM | 654 | C | GLU | 96 | −2.810 | 39.327 | 58.519 | 1.00 | 88.40 |
| ATOM | 655 | O | GLU | 96 | −2.097 | 40.166 | 59.052 | 1.00 | 89.12 |
| ATOM | 656 | N | GLY | 97 | −2.431 | 38.700 | 57.404 | 1.00 | 86.87 |
| ATOM | 657 | CA | GLY | 97 | −1.133 | 38.917 | 56.789 | 1.00 | 85.05 |
| ATOM | 658 | C | GLY | 97 | −0.161 | 37.976 | 57.494 | 1.00 | 84.17 |
| ATOM | 659 | O | GLY | 97 | 1.044 | 38.179 | 57.605 | 1.00 | 83.49 |
| ATOM | 660 | N | GLN | 98 | −0.820 | 36.901 | 57.977 | 1.00 | 83.07 |
| ATOM | 661 | CA | GLN | 98 | −0.253 | 35.810 | 58.769 | 1.00 | 82.28 |
| ATOM | 662 | CB | GLN | 98 | −1.346 | 34.825 | 59.250 | 1.00 | 82.41 |
| ATOM | 663 | CG | GLN | 98 | −2.647 | 35.462 | 59.699 | 1.00 | 83.61 |
| ATOM | 664 | CD | GLN | 98 | −3.740 | 34.427 | 60.007 | 1.00 | 84.16 |
| ATOM | 665 | OE1 | GLN | 98 | −3.606 | 33.239 | 59.714 | 1.00 | 84.01 |
| ATOM | 666 | NE2 | GLN | 98 | −4.905 | 34.685 | 60.592 | 1.00 | 84.46 |
| ATOM | 667 | C | GLN | 98 | 0.735 | 34.981 | 58.011 | 1.00 | 81.85 |
| ATOM | 668 | O | GLN | 98 | 1.955 | 35.200 | 57.956 | 1.00 | 83.51 |
| ATOM | 669 | N | TRP | 99 | 0.118 | 33.962 | 57.470 | 1.00 | 79.05 |
| ATOM | 670 | CA | TRP | 99 | 0.703 | 32.914 | 56.706 | 1.00 | 75.85 |
| ATOM | 671 | CB | TRP | 99 | 1.993 | 32.398 | 57.308 | 1.00 | 73.88 |
| ATOM | 672 | CG | TRP | 99 | 2.968 | 31.780 | 56.325 | 1.00 | 71.82 |
| ATOM | 673 | CD2 | TRP | 99 | 3.211 | 30.386 | 56.075 | 1.00 | 70.49 |
| ATOM | 674 | CE2 | TRP | 99 | 4.222 | 30.308 | 55.123 | 1.00 | 69.72 |
| ATOM | 675 | CE3 | TRP | 99 | 2.671 | 29.200 | 56.550 | 1.00 | 69.52 |
| ATOM | 676 | CD1 | TRP | 99 | 3.832 | 32.464 | 55.525 | 1.00 | 71.99 |
| ATOM | 677 | NE1 | TRP | 99 | 4.598 | 31.589 | 54.790 | 1.00 | 71.07 |
| ATOM | 678 | CZ2 | TRP | 99 | 4.692 | 29.089 | 54.624 | 1.00 | 67.81 |
| ATOM | 679 | CZ3 | TRP | 99 | 3.136 | 27.984 | 56.080 | 1.00 | 67.31 |
| ATOM | 680 | CH2 | TRP | 99 | 4.151 | 27.945 | 55.111 | 1.00 | 67.77 |
| ATOM | 681 | C | TRP | 99 | −0.247 | 31.793 | 56.673 | 1.00 | 74.58 |
| ATOM | 682 | O | TRP | 99 | −1.060 | 31.567 | 57.556 | 1.00 | 75.00 |
| ATOM | 683 | N | SER | 100 | −0.090 | 31.087 | 55.647 | 1.00 | 72.11 |
| ATOM | 684 | CA | SER | 100 | −0.948 | 29.999 | 55.517 | 1.00 | 68.48 |
| ATOM | 685 | CB | SER | 100 | −2.376 | 30.466 | 55.232 | 1.00 | 68.40 |
| ATOM | 686 | OG | SER | 100 | −2.467 | 31.128 | 53.985 | 1.00 | 68.76 |
| ATOM | 687 | C | SER | 100 | −0.522 | 29.152 | 54.382 | 1.00 | 66.28 |
| ATOM | 688 | O | SER | 100 | 0.405 | 29.473 | 53.632 | 1.00 | 65.13 |
| ATOM | 689 | N | VAL | 101 | −1.225 | 28.028 | 54.291 | 1.00 | 64.27 |
| ATOM | 690 | CA | VAL | 101 | −0.982 | 27.030 | 53.262 | 1.00 | 62.66 |
| ATOM | 691 | CB | VAL | 101 | 0.090 | 26.023 | 53.715 | 1.00 | 62.98 |
| ATOM | 692 | CG1 | VAL | 101 | 1.493 | 26.554 | 53.459 | 1.00 | 66.77 |
| ATOM | 693 | CG2 | VAL | 101 | −0.075 | 25.688 | 55.198 | 1.00 | 63.17 |
| ATOM | 694 | C | VAL | 101 | −2.219 | 26.243 | 52.878 | 1.00 | 60.88 |
| ATOM | 695 | O | VAL | 101 | −2.561 | 25.258 | 53.530 | 1.00 | 60.62 |
| ATOM | 696 | N | LYS | 102 | −2.880 | 26.671 | 51.810 | 1.00 | 58.24 |
| ATOM | 697 | CA | LYS | 102 | −4.066 | 25.981 | 51.337 | 1.00 | 56.12 |
| ATOM | 698 | CB | LYS | 102 | −4.887 | 26.880 | 50.410 | 1.00 | 57.06 |
| ATOM | 699 | CG | LYS | 102 | −5.884 | 27.806 | 51.111 | 1.00 | 60.55 |
| ATOM | 700 | CD | LYS | 102 | −7.056 | 27.038 | 51.748 | 1.00 | 63.17 |
| ATOM | 701 | CE | LYS | 102 | −8.282 | 27.944 | 52.036 | 1.00 | 64.70 |
| ATOM | 702 | NZ | LYS | 102 | −8.021 | 29.150 | 52.899 | 1.00 | 63.52 |
| ATOM | 703 | C | LYS | 102 | −3.677 | 24.710 | 50.596 | 1.00 | 54.04 |
| ATOM | 704 | O | LYS | 102 | −2.599 | 24.609 | 50.007 | 1.00 | 52.35 |
| ATOM | 705 | N | THR | 103 | −4.576 | 23.738 | 50.631 | 1.00 | 52.24 |
| ATOM | 706 | CA | THR | 103 | −4.345 | 22.474 | 49.972 | 1.00 | 49.72 |
| ATOM | 707 | CB | THR | 103 | −4.139 | 21.385 | 51.010 | 1.00 | 49.49 |
| ATOM | 708 | OG1 | THR | 103 | −3.399 | 20.316 | 50.422 | 1.00 | 53.11 |
| ATOM | 709 | CG2 | THR | 103 | −5.475 | 20.861 | 51.517 | 1.00 | 48.32 |
| ATOM | 710 | C | THR | 103 | −5.563 | 22.158 | 49.106 | 1.00 | 49.61 |
| ATOM | 711 | O | THR | 103 | −6.693 | 22.435 | 49.507 | 1.00 | 50.24 |
| ATOM | 712 | N | LYS | 104 | −5.330 | 21.587 | 47.924 | 1.00 | 48.56 |
| ATOM | 713 | CA | LYS | 104 | −6.404 | 21.251 | 46.983 | 1.00 | 48.50 |
| ATOM | 714 | CB | LYS | 104 | −6.469 | 22.298 | 45.864 | 1.00 | 49.98 |
| ATOM | 715 | CG | LYS | 104 | −6.753 | 23.737 | 46.313 | 1.00 | 56.05 |
| ATOM | 716 | CD | LYS | 104 | −8.195 | 23.932 | 46.814 | 1.00 | 60.38 |
| ATOM | 717 | CE | LYS | 104 | −8.456 | 25.383 | 47.254 | 1.00 | 62.32 |
| ATOM | 718 | NZ | LYS | 104 | −9.845 | 25.649 | 47.761 | 1.00 | 61.31 |
| ATOM | 719 | C | LYS | 104 | −6.224 | 19.878 | 46.332 | 1.00 | 48.13 |
| ATOM | 720 | O | LYS | 104 | −5.286 | 19.685 | 45.563 | 1.00 | 49.60 |
| ATOM | 721 | N | HIS | 105 | −7.127 | 18.936 | 46.606 | 1.00 | 47.57 |
| ATOM | 722 | CA | HIS | 105 | −7.023 | 17.601 | 46.101 | 1.00 | 47.23 |
| ATOM | 723 | CB | HIS | 105 | −7.165 | 16.529 | 47.074 | 1.00 | 47.40 |
| ATOM | 724 | CG | HIS | 105 | −6.241 | 16.709 | 48.228 | 1.00 | 49.37 |
| ATOM | 725 | CD2 | HIS | 105 | −5.098 | 16.066 | 48.563 | 1.00 | 49.55 |
| ATOM | 726 | ND1 | HIS | 105 | −6.459 | 17.648 | 49.212 | 1.00 | 50.43 |
| ATOM | 727 | CE1 | HIS | 105 | −5.493 | 17.571 | 50.110 | 1.00 | 51.38 |
| ATOM | 728 | NE2 | HIS | 105 | −4.655 | 16.619 | 49.740 | 1.00 | 50.58 |
| ATOM | 729 | C | HIS | 105 | −8.030 | 17.304 | 44.907 | 1.00 | 46.39 |
| ATOM | 730 | O | HIS | 105 | −9.195 | 17.692 | 44.985 | 1.00 | 49.62 |
| ATOM | 731 | N | GLN | 106 | −7.575 | 16.580 | 43.894 | 1.00 | 42.98 |
| ATOM | 732 | CA | GLN | 106 | −8.419 | 16.226 | 42.771 | 1.00 | 40.44 |
| ATOM | 733 | CB | GLN | 106 | −8.284 | 17.285 | 41.685 | 1.00 | 40.41 |
| ATOM | 734 | CG | GLN | 106 | −9.546 | 17.548 | 40.908 | 1.00 | 40.59 |
| ATOM | 735 | CD | GLN | 106 | −10.428 | 16.324 | 40.813 | 1.00 | 40.54 |
| ATOM | 736 | OE1 | GLN | 106 | −11.061 | 15.927 | 41.795 | 1.00 | 39.16 |
| ATOM | 737 | NE2 | GLN | 106 | −10.475 | 15.712 | 39.631 | 1.00 | 40.06 |
| ATOM | 738 | C | GLN | 106 | −7.940 | 14.878 | 42.249 | 1.00 | 40.70 |
| ATOM | 739 | O | GLN | 106 | −6.745 | 14.699 | 42.012 | 1.00 | 41.69 |
| ATOM | 740 | N | MET | 107 | −8.867 | 13.937 | 42.066 | 1.00 | 41.01 |
| ATOM | 741 | CA | MET | 107 | −8.532 | 12.588 | 41.599 | 1.00 | 40.17 |
| ATOM | 742 | CB | MET | 107 | −9.083 | 11.551 | 42.588 | 1.00 | 42.07 |
| ATOM | 743 | CG | MET | 107 | −8.772 | 10.094 | 42.249 | 1.00 | 44.67 |
| ATOM | 744 | SD | MET | 107 | −10.185 | 9.202 | 41.551 | 1.00 | 50.71 |
| ATOM | 745 | CE | MET | 107 | −10.688 | 8.056 | 42.927 | 1.00 | 43.37 |
| ATOM | 746 | C | MET | 107 | −9.059 | 12.294 | 40.204 | 1.00 | 38.93 |
| ATOM | 747 | O | MET | 107 | −10.264 | 12.285 | 39.979 | 1.00 | 41.30 |
| ATOM | 748 | N | TYR | 108 | −8.161 | 12.044 | 39.264 | 1.00 | 37.96 |
| ATOM | 749 | CA | TYR | 108 | −8.588 | 11.750 | 37.907 | 1.00 | 38.48 |
| ATOM | 750 | CB | TYR | 108 | −7.670 | 12.454 | 36.900 | 1.00 | 35.63 |
| ATOM | 751 | CG | TYR | 108 | −7.732 | 13.972 | 36.977 | 1.00 | 35.18 |
| ATOM | 752 | CD1 | TYR | 108 | −7.492 | 14.645 | 38.180 | 1.00 | 37.21 |
| ATOM | 753 | CE1 | TYR | 108 | −7.550 | 16.047 | 38.268 | 1.00 | 34.81 |
| ATOM | 754 | CD2 | TYR | 108 | −8.031 | 14.735 | 35.857 | 1.00 | 34.14 |
| ATOM | 755 | CE2 | TYR | 108 | −8.092 | 16.134 | 35.931 | 1.00 | 35.09 |
| ATOM | 756 | CZ | TYR | 108 | −7.852 | 16.783 | 37.139 | 1.00 | 35.25 |
| ATOM | 757 | OH | TYR | 108 | −7.937 | 18.158 | 37.211 | 1.00 | 33.27 |
| ATOM | 758 | C | TYR | 108 | −8.583 | 10.241 | 37.689 | 1.00 | 40.17 |
| ATOM | 759 | O | TYR | 108 | −7.817 | 9.514 | 38.325 | 1.00 | 38.04 |
| ATOM | 760 | N | SER | 109 | −9.469 | 9.765 | 36.818 | 1.00 | 42.63 |
| ATOM | 761 | CA | SER | 109 | −9.524 | 8.341 | 36.530 | 1.00 | 44.60 |
| ATOM | 762 | CB | SER | 109 | −10.929 | 7.787 | 36.736 | 1.00 | 43.05 |
| ATOM | 763 | OG | SER | 109 | −10.926 | 6.385 | 36.522 | 1.00 | 41.66 |
| ATOM | 746 | C | SER | 109 | −9.090 | 8.106 | 35.097 | 1.00 | 46.74 |
| ATOM | 765 | O | SER | 109 | −9.531 | 8.799 | 34.182 | 1.00 | 44.65 |
| ATOM | 766 | N | ILE | 110 | −8.217 | 7.120 | 34.918 | 1.00 | 50.31 |
| ATOM | 767 | CA | ILE | 110 | −7.686 | 6.782 | 33.608 | 1.00 | 55.29 |
| ATOM | 768 | CB | ILE | 110 | −6.326 | 6.060 | 33.731 | 1.00 | 54.32 |
| ATOM | 769 | CG2 | ILE | 110 | −5.690 | 5.932 | 32.364 | 1.00 | 56.16 |
| ATOM | 770 | CG1 | ILE | 110 | −5.373 | 6.844 | 34.626 | 1.00 | 53.30 |
| ATOM | 771 | CD1 | ILE | 110 | −4.067 | 6.117 | 34.869 | 1.00 | 51.57 |
| ATOM | 772 | C | ILE | 110 | −8.621 | 5.882 | 32.799 | 1.00 | 59.70 |
| ATOM | 773 | O | ILE | 110 | −8.906 | 4.749 | 33.199 | 1.00 | 58.82 |
| ATOM | 774 | N | PRO | 111 | −9.114 | 6.381 | 31.650 | 1.00 | 64.10 |
| ATOM | 775 | CD | PRO | 111 | −8.972 | 7.759 | 31.142 | 1.00 | 64.05 |
| ATOM | 776 | CA | PRO | 111 | −10.012 | 5.608 | 30.788 | 1.00 | 68.40 |
| ATOM | 777 | CB | PRO | 111 | −10.118 | 6.484 | 29.547 | 1.00 | 67.29 |
| ATOM | 778 | CG | PRO | 111 | −10.105 | 7.860 | 30.144 | 1.00 | 63.88 |
| ATOM | 779 | C | PRO | 111 | −9.416 | 4.231 | 30.494 | 1.00 | 72.88 |
| ATOM | 780 | O | PRO | 111 | −8.195 | 4.065 | 30.506 | 1.00 | 73.72 |
| ATOM | 781 | N | GLU | 112 | −10.280 | 3.250 | 30.239 | 1.00 | 77.60 |
| ATOM | 782 | CA | GLU | 112 | −9.845 | 1.879 | 29.958 | 1.00 | 80.79 |
| ATOM | 783 | CB | GLU | 112 | −11.072 | 0.968 | 29.798 | 1.00 | 82.29 |
| ATOM | 784 | CG | GLU | 112 | −10.748 | −0.498 | 29.524 | 1.00 | 83.62 |
| ATOM | 785 | CD | GLU | 112 | −11.896 | −1.247 | 28.851 | 1.00 | 85.04 |
| ATOM | 786 | OE1 | GLU | 112 | −11.697 | −2.423 | 28.470 | 1.00 | 85.60 |
| ATOM | 787 | OE2 | GLU | 112 | −12.995 | −0.665 | 28.700 | 1.00 | 85.42 |
| ATOM | 788 | C | GLU | 112 | −8.971 | 1.806 | 28.702 | 1.00 | 82.21 |
| ATOM | 789 | O | GLU | 112 | −7.936 | 1.137 | 28.693 | 1.00 | 82.17 |
| ATOM | 790 | N | ASP | 113 | −9.394 | 2.501 | 27.649 | 1.00 | 83.97 |
| ATOM | 791 | CA | ASP | 113 | −8.660 | 2.522 | 26.385 | 1.00 | 85.79 |
| ATOM | 792 | CB | ASP | 113 | −9.506 | 3.221 | 25.302 | 1.00 | 86.45 |
| ATOM | 793 | CG | ASP | 113 | −9.961 | 4.624 | 25.712 | 1.00 | 87.32 |
| ATOM | 794 | OD1 | ASP | 113 | −10.655 | 4.756 | 26.748 | 1.00 | 86.75 |
| ATOM | 795 | OD2 | ASP | 113 | −9.629 | 5.595 | 24.991 | 1.00 | 87.18 |
| ATOM | 769 | C | ASP | 113 | −7.297 | 3.215 | 26.533 | 1.00 | 86.44 |
| ATOM | 797 | O | ASP | 113 | −6.467 | 3.195 | 25.617 | 1.00 | 86.35 |
| ATOM | 798 | N | ALA | 114 | −7.075 | 3.813 | 27.701 | 1.00 | 86.34 |
| ATOM | 799 | CA | ALA | 114 | −5.837 | 4.533 | 28.000 | 1.00 | 85.22 |
| ATOM | 800 | CB | ALA | 114 | −6.174 | 5.904 | 28.585 | 1.00 | 84.46 |
| ATOM | 801 | C | ALA | 114 | −4.928 | 3.768 | 28.963 | 1.00 | 83.67 |
| ATOM | 802 | O | ALA | 114 | −3.716 | 3.692 | 28.762 | 1.00 | 83.48 |
| ATOM | 803 | N | MET | 115 | −5.528 | 3.212 | 30.012 | 1.00 | 81.79 |
| ATOM | 804 | CA | MET | 115 | −4.802 | 2.457 | 31.023 | 1.00 | 78.70 |
| ATOM | 805 | CB | MET | 115 | −5.776 | 2.050 | 32.135 | 1.00 | 81.16 |
| ATOM | 806 | CG | MET | 115 | −5.148 | 1.863 | 33.503 | 1.00 | 84.52 |
| ATOM | 807 | SD | MET | 115 | −3.978 | 0.492 | 33.553 | 1.00 | 90.44 |
| ATOM | 808 | CE | MET | 115 | −5.060 | −0.891 | 34.119 | 1.00 | 88.49 |
| ATOM | 809 | C | MET | 115 | −4.145 | 1.224 | 30.391 | 1.00 | 76.27 |
| ATOM | 810 | O | MET | 115 | −3.066 | 0.809 | 30.813 | 1.00 | 74.47 |
| ATOM | 811 | N | THR | 116 | −4.796 | 0.658 | 29.372 | 1.00 | 74.50 |
| ATOM | 812 | CA | THR | 116 | −4.282 | −0.518 | 28.666 | 1.00 | 72.46 |
| ATOM | 813 | CB | THR | 116 | −5.399 | −1.524 | 28.309 | 1.00 | 72.22 |
| ATOM | 814 | OG1 | THR | 116 | −6.200 | −0.993 | 27.244 | 1.00 | 71.17 |
| ATOM | 815 | CG2 | THR | 116 | −6.275 | −1.805 | 29.516 | 1.00 | 71.94 |
| ATOM | 816 | C | THR | 116 | −3.621 | −0.110 | 27.356 | 1.00 | 71.75 |
| ATOM | 817 | O | THR | 116 | −3.562 | −0.899 | 26.412 | 1.00 | 71.39 |
| ATOM | 818 | N | GLY | 117 | −3.142 | 1.131 | 27.301 | 1.00 | 71.09 |
| ATOM | 819 | CA | GLY | 117 | −2.477 | 1.639 | 26.110 | 1.00 | 68.62 |
| ATOM | 820 | C | GLY | 117 | −0.961 | 1.651 | 26.260 | 1.00 | 66.70 |
| ATOM | 821 | O | GLY | 117 | −0.384 | 0.702 | 26.798 | 1.00 | 67.20 |
| ATOM | 822 | N | THR | 118 | −0.313 | 2.716 | 25.783 | 1.00 | 63.05 |
| ATOM | 823 | CA | THR | 118 | 1.142 | 2.844 | 25.876 | 1.00 | 59.92 |
| ATOM | 824 | CB | THR | 118 | 1.796 | 3.020 | 24.502 | 1.00 | 59.06 |
| ATOM | 825 | OG1 | THR | 118 | 1.013 | 3.926 | 23.718 | 1.00 | 57.88 |
| ATOM | 826 | CG2 | THR | 118 | 1.917 | 1.688 | 23.794 | 1.00 | 59.21 |
| ATOM | 827 | C | THR | 118 | 1.548 | 4.038 | 26.721 | 1.00 | 58.97 |
| ATOM | 828 | O | THR | 118 | 0.764 | 4.971 | 26.912 | 1.00 | 58.11 |
| ATOM | 829 | N | ALA | 119 | 2.782 | 4.001 | 27.218 | 1.00 | 56.72 |
| ATOM | 830 | CA | ALA | 119 | 3.313 | 5.071 | 28.052 | 1.00 | 52.86 |
| ATOM | 831 | CB | ALA | 119 | 4.807 | 4.938 | 28.177 | 1.00 | 51.30 |
| ATOM | 832 | C | ALA | 119 | 2.972 | 6.399 | 27.421 | 1.00 | 51.58 |
| ATOM | 833 | O | ALA | 119 | 2.456 | 7.301 | 28.080 | 1.00 | 52.70 |
| ATOM | 834 | N | GLU | 120 | 3.260 | 6.502 | 26.131 | 1.00 | 48.02 |
| ATOM | 835 | CA | GLU | 120 | 2.994 | 7.716 | 25.386 | 1.00 | 46.07 |
| ATOM | 836 | CB | GLU | 120 | 3.194 | 7.471 | 23.894 | 1.00 | 49.10 |
| ATOM | 837 | CG | GLU | 120 | 4.210 | 6.381 | 23.550 | 1.00 | 52.89 |
| ATOM | 838 | CD | GLU | 120 | 5.630 | 6.736 | 23.945 | 1.00 | 53.64 |
| ATOM | 839 | OE1 | GLU | 120 | 5.962 | 6.621 | 25.141 | 1.00 | 55.30 |
| ATOM | 840 | OE2 | GLU | 120 | 6.411 | 7.139 | 23.057 | 1.00 | 52.83 |
| ATOM | 841 | C | GLU | 120 | 1.557 | 8.140 | 25.630 | 1.00 | 44.27 |
| ATOM | 842 | O | GLU | 120 | 1.295 | 9.257 | 26.070 | 1.00 | 44.84 |
| ATOM | 843 | N | MET | 121 | 0.627 | 7.235 | 25.351 | 1.00 | 41.37 |
| ATOM | 844 | CA | MET | 121 | −0.791 | 7.525 | 25.513 | 1.00 | 38.57 |
| ATOM | 845 | CB | MET | 121 | −1.626 | 6.358 | 24.990 | 1.00 | 41.30 |
| ATOM | 846 | CG | MET | 121 | −1.721 | 6.328 | 23.479 | 1.00 | 46.24 |
| ATOM | 847 | SD | MET | 121 | −2.483 | 4.835 | 22.838 | 1.00 | 50.88 |
| ATOM | 848 | CE | MET | 121 | −3.908 | 4.669 | 23.961 | 1.00 | 50.02 |
| ATOM | 849 | C | MET | 121 | −1.190 | 7.820 | 26.937 | 1.00 | 34.60 |
| ATOM | 850 | O | MET | 121 | −1.910 | 8.780 | 27.204 | 1.00 | 31.69 |
| ATOM | 851 | N | LEU | 122 | −0.719 | 6.985 | 27.852 | 1.00 | 32.63 |
| ATOM | 852 | CA | LEU | 122 | −1.051 | 7.141 | 29.263 | 1.00 | 30.24 |
| ATOM | 853 | CB | LEU | 122 | −0.256 | 6.140 | 30.108 | 1.00 | 27.33 |
| ATOM | 854 | CG | LEU | 122 | −0.778 | 5.923 | 31.533 | 1.00 | 21.99 |
| ATOM | 855 | CD1 | LEU | 122 | −0.279 | 4.601 | 32.031 | 1.00 | 22.53 |
| ATOM | 856 | CD2 | LEU | 122 | −0.366 | 7.034 | 32.456 | 1.00 | 17.78 |
| ATOM | 857 | C | LEU | 122 | −0.759 | 8.551 | 29.746 | 1.00 | 28.67 |
| ATOM | 858 | O | LEU | 122 | −1.619 | 9.228 | 30.326 | 1.00 | 25.21 |
| ATOM | 859 | N | PHE | 123 | 0.469 | 8.987 | 29.502 | 1.00 | 26.83 |
| ATOM | 860 | CA | PHE | 123 | 0.871 | 10.306 | 29.929 | 1.00 | 25.29 |
| ATOM | 861 | CB | PHE | 123 | 2.387 | 10.398 | 29.908 | 1.00 | 20.22 |
| ATOM | 862 | CG | PHE | 123 | 3.015 | 9.772 | 31.112 | 1.00 | 15.51 |
| ATOM | 863 | CD1 | PHE | 123 | 3.538 | 8.494 | 31.064 | 1.00 | 12.96 |
| ATOM | 864 | CD2 | PHE | 123 | 3.028 | 10.457 | 32.328 | 1.00 | 13.35 |
| ATOM | 865 | CE1 | PHE | 123 | 4.067 | 7.910 | 32.217 | 1.00 | 12.87 |
| ATOM | 866 | CE2 | PHE | 123 | 3.552 | 9.879 | 33.484 | 1.00 | 9.69 |
| ATOM | 867 | CZ | PHE | 123 | 4.072 | 8.609 | 33.432 | 1.00 | 9.56 |
| ATOM | 868 | C | PHE | 123 | 0.202 | 11.432 | 29.157 | 1.00 | 26.20 |
| ATOM | 869 | O | PHE | 123 | −0.102 | 12.489 | 29.722 | 1.00 | 26.61 |
| ATOM | 870 | N | ASP | 124 | −0.053 | 11.207 | 27.875 | 1.00 | 24.47 |
| ATOM | 871 | CA | ASP | 124 | −0.750 | 12.210 | 27.090 | 1.00 | 23.14 |
| ATOM | 872 | CB | ASP | 124 | −1.228 | 11.614 | 25.785 | 1.00 | 24.52 |
| ATOM | 873 | CG | ASP | 124 | −0.178 | 11.628 | 24.747 | 1.00 | 27.01 |
| ATOM | 874 | OD1 | ASP | 124 | −0.376 | 10.955 | 23.715 | 1.00 | 26.39 |
| ATOM | 875 | OD2 | ASP | 124 | 0.839 | 12.325 | 24.968 | 1.00 | 29.23 |
| ATOM | 876 | C | ASP | 124 | −1.967 | 12.650 | 27.875 | 1.00 | 21.89 |
| ATOM | 877 | O | ASP | 124 | −2.361 | 13.815 | 27.841 | 1.00 | 20.01 |
| ATOM | 878 | N | TYR | 125 | −2.562 | 11.688 | 28.574 | 1.00 | 20.84 |
| ATOM | 879 | CA | TYR | 125 | −3.749 | 11.943 | 29.371 | 1.00 | 20.51 |
| ATOM | 880 | CB | TYR | 125 | −4.414 | 10.619 | 29.792 | 1.00 | 20.43 |
| ATOM | 881 | CG | TYR | 125 | −5.796 | 10.794 | 30.394 | 1.00 | 22.84 |
| ATOM | 882 | CD1 | TYR | 125 | −6.083 | 10.358 | 31.692 | 1.00 | 23.51 |
| ATOM | 883 | CE1 | TYR | 125 | −7.345 | 10.584 | 32.268 | 1.00 | 31.08 |
| ATOM | 884 | CD2 | TYR | 125 | −6.803 | 11.451 | 29.678 | 1.00 | 26.43 |
| ATOM | 885 | CE2 | TYR | 125 | −8.064 | 11.685 | 30.232 | 1.00 | 31.61 |
| ATOM | 886 | CZ | TYR | 125 | −8.336 | 11.255 | 31.528 | 1.00 | 34.64 |
| ATOM | 887 | OH | TYR | 125 | −9.585 | 11.520 | 32.073 | 1.00 | 38.10 |
| ATOM | 888 | C | TYR | 125 | −3.382 | 12.752 | 30.605 | 1.00 | 19.11 |
| ATOM | 889 | O | TYR | 125 | −3.904 | 13.848 | 30.824 | 1.00 | 16.08 |
| ATOM | 890 | N | ILE | 126 | −2.465 | 12.212 | 31.399 | 1.00 | 17.91 |
| ATOM | 891 | CA | ILE | 126 | −2.049 | 12.879 | 32.615 | 1.00 | 17.82 |
| ATOM | 892 | CB | ILE | 126 | −0.819 | 12.236 | 33.203 | 1.00 | 19.82 |
| ATOM | 893 | CG2 | ILE | 126 | −0.489 | 12.905 | 34.538 | 1.00 | 18.77 |
| ATOM | 894 | CG1 | ILE | 126 | −1.055 | 10.732 | 33.331 | 1.00 | 21.27 |
| ATOM | 895 | CD1 | ILE | 126 | 0.045 | 9.984 | 34.062 | 1.00 | 23.92 |
| ATOM | 896 | C | ILE | 126 | −1.717 | 14.313 | 32.325 | 1.00 | 18.09 |
| ATOM | 897 | O | ILE | 126 | −1.991 | 15.205 | 33.123 | 1.00 | 16.68 |
| ATOM | 898 | N | SER | 127 | −1.108 | 14.532 | 31.172 | 1.00 | 19.12 |
| ATOM | 899 | CA | SER | 127 | −0.747 | 15.877 | 30.789 | 1.00 | 20.96 |
| ATOM | 900 | CB | SER | 127 | −0.057 | 15.857 | 29.432 | 1.00 | 19.89 |
| ATOM | 901 | OG | SER | 127 | 0.569 | 17.100 | 29.190 | 1.00 | 22.20 |
| ATOM | 902 | C | SER | 127 | −2.011 | 16.742 | 30.746 | 1.00 | 21.92 |
| ATOM | 903 | O | SER | 127 | −2.177 | 17.658 | 31.551 | 1.00 | 20.25 |
| ATOM | 904 | N | GLU | 128 | −2.902 | 16.431 | 29.813 | 1.00 | 23.87 |
| ATOM | 905 | CA | GLU | 128 | −4.152 | 17.161 | 29.670 | 1.00 | 26.98 |
| ATOM | 906 | CB | GLU | 128 | −5.111 | 16.353 | 28.802 | 1.00 | 33.10 |
| ATOM | 907 | CG | GLU | 128 | −6.471 | 16.990 | 28.544 | 1.00 | 39.51 |
| ATOM | 908 | CD | GLU | 128 | −7.280 | 16.175 | 27.544 | 1.00 | 44.52 |
| ATOM | 909 | OE1 | GLU | 128 | −7.211 | 16.481 | 26.327 | 1.00 | 46.11 |
| ATOM | 910 | OE2 | GLU | 128 | −7.963 | 15.218 | 27.980 | 1.00 | 43.93 |
| ATOM | 911 | C | GLU | 128 | −4.797 | 17.431 | 31.020 | 1.00 | 26.55 |
| ATOM | 912 | O | GLU | 128 | −5.177 | 18.561 | 31.334 | 1.00 | 26.16 |
| ATOM | 913 | N | CYS | 129 | −4.929 | 16.384 | 31.820 | 1.00 | 26.36 |
| ATOM | 914 | CA | CYS | 129 | −5.532 | 16.535 | 33.130 | 1.00 | 26.47 |
| ATOM | 915 | CB | CYS | 129 | −5.452 | 15.219 | 33.893 | 1.00 | 28.39 |
| ATOM | 916 | SG | CYS | 129 | −6.450 | 13.922 | 33.126 | 1.00 | 37.58 |
| ATOM | 917 | C | CYS | 129 | −4.853 | 17.636 | 33.914 | 1.00 | 25.00 |
| ATOM | 918 | O | CYS | 129 | −5.515 | 18.561 | 34.372 | 1.00 | 24.97 |
| ATOM | 919 | N | ILE | 130 | −3.532 | 17.536 | 34.059 | 1.00 | 24.74 |
| ATOM | 920 | CA | ILE | 130 | −2.763 | 18.536 | 34.793 | 1.00 | 21.55 |
| ATOM | 921 | CB | ILE | 130 | −1.245 | 18.255 | 34.709 | 1.00 | 17.55 |
| ATOM | 922 | CG2 | ILE | 130 | −0.458 | 19.404 | 35.304 | 1.00 | 15.00 |
| ATOM | 923 | CG1 | ILE | 130 | −0.915 | 16.984 | 35.490 | 1.00 | 16.42 |
| ATOM | 924 | CD1 | ILE | 130 | 0.574 | 16.713 | 35.623 | 1.00 | 18.34 |
| ATOM | 925 | C | ILE | 130 | −3.070 | 19.910 | 34.219 | 1.00 | 23.54 |
| ATOM | 926 | O | ILE | 130 | −3.572 | 20.780 | 34.926 | 1.00 | 21.27 |
| ATOM | 927 | N | SER | 131 | −2.785 | 20.091 | 32.933 | 1.00 | 26.25 |
| ATOM | 928 | CA | SER | 131 | −3.048 | 21.353 | 32.270 | 1.00 | 28.50 |
| ATOM | 929 | CB | SER | 131 | −3.011 | 21.186 | 30.764 | 1.00 | 28.76 |
| ATOM | 930 | OG | SER | 131 | −3.856 | 22.154 | 30.164 | 1.00 | 32.87 |
| ATOM | 931 | C | SER | 131 | −4.417 | 21.851 | 32.661 | 1.00 | 31.48 |
| ATOM | 932 | O | SER | 131 | −4.586 | 23.002 | 33.057 | 1.00 | 33.67 |
| ATOM | 933 | N | ASP | 132 | −5.411 | 20.986 | 32.546 | 1.00 | 34.56 |
| ATOM | 934 | CA | ASP | 132 | −6.753 | 21.397 | 32.908 | 1.00 | 39.04 |
| ATOM | 935 | CB | ASP | 132 | −7.735 | 20.248 | 32.694 | 1.00 | 44.84 |
| ATOM | 936 | CG | ASP | 132 | −9.165 | 20.650 | 32.987 | 1.00 | 50.51 |
| ATOM | 937 | OD1 | ASP | 132 | −9.764 | 21.347 | 32.131 | 1.00 | 53.56 |
| ATOM | 938 | OD2 | ASP | 132 | −9.674 | 20.283 | 34.078 | 1.00 | 52.37 |
| ATOM | 939 | C | ASP | 132 | −6.790 | 21.843 | 34.376 | 1.00 | 38.23 |
| ATOM | 940 | O | ASP | 132 | −7.160 | 22.982 | 34.677 | 1.00 | 36.81 |
| ATOM | 941 | N | PHE | 133 | −6.394 | 20.932 | 35.270 | 1.00 | 36.88 |
| ATOM | 942 | CA | PHE | 133 | −6.372 | 21.170 | 36.713 | 1.00 | 34.85 |
| ATOM | 943 | CB | PHE | 133 | −5.604 | 20.060 | 37.433 | 1.00 | 33.59 |
| ATOM | 944 | CG | PHE | 133 | −5.343 | 20.362 | 38.878 | 1.00 | 34.77 |
| ATOM | 945 | CD1 | PHE | 133 | −6.396 | 20.547 | 39.760 | 1.00 | 35.58 |
| ATOM | 946 | CD2 | PHE | 133 | −4.043 | 20.523 | 39.348 | 1.00 | 37.81 |
| ATOM | 947 | CE1 | PHE | 133 | −6.159 | 20.896 | 41.091 | 1.00 | 37.66 |
| ATOM | 948 | CE2 | PHE | 133 | −3.792 | 20.872 | 40.678 | 1.00 | 38.00 |
| ATOM | 949 | CZ | PHE | 133 | −4.850 | 21.059 | 41.548 | 1.00 | 38.85 |
| ATOM | 950 | C | PHE | 133 | −5.755 | 22.503 | 37.094 | 1.00 | 34.28 |
| ATOM | 951 | O | PHE | 133 | −6.274 | 23.226 | 37.947 | 1.00 | 33.97 |
| ATOM | 952 | N | LEU | 134 | −4.622 | 22.813 | 36.482 | 1.00 | 33.97 |
| ATOM | 953 | CA | LEU | 134 | −3.958 | 24.070 | 36.766 | 1.00 | 31.79 |
| ATOM | 954 | CB | LEU | 134 | −2.590 | 24.109 | 36.089 | 1.00 | 24.12 |
| ATOM | 955 | CG | LEU | 134 | −1.618 | 23.026 | 36.545 | 1.00 | 16.64 |
| ATOM | 956 | CD1 | LEU | 134 | −0.368 | 23.101 | 35.705 | 1.00 | 15.98 |
| ATOM | 957 | CD2 | LEU | 134 | −1.305 | 23.184 | 38.014 | 1.00 | 10.77 |
| ATOM | 958 | C | LEU | 134 | −4.855 | 25.176 | 36.234 | 1.00 | 34.44 |
| ATOM | 959 | O | LEU | 134 | −5.111 | 26.163 | 36.920 | 1.00 | 34.41 |
| ATOM | 960 | N | ASP | 135 | −5.365 | 24.999 | 35.022 | 1.00 | 37.26 |
| ATOM | 961 | CA | ASP | 135 | −6.230 | 26.014 | 34.454 | 1.00 | 42.65 |
| ATOM | 962 | CB | ASP | 135 | −6.815 | 25.565 | 33.121 | 1.00 | 46.76 |
| ATOM | 963 | CG | ASP | 135 | −7.707 | 26.629 | 32.509 | 1.00 | 52.18 |
| ATOM | 964 | OD1 | ASP | 135 | −8.659 | 26.271 | 31.772 | 1.00 | 53.75 |
| ATOM | 965 | OD2 | ASP | 135 | −7.443 | 27.829 | 32.772 | 1.00 | 52.70 |
| ATOM | 966 | C | ASP | 135 | −7.386 | 26.381 | 35.383 | 1.00 | 43.96 |
| ATOM | 967 | O | ASP | 135 | −7.643 | 27.563 | 35.619 | 1.00 | 44.98 |
| ATOM | 968 | N | LYS | 136 | −8.084 | 25.368 | 35.894 | 1.00 | 44.30 |
| ATOM | 969 | CA | LYS | 136 | −9.225 | 25.578 | 36.780 | 1.00 | 44.56 |
| ATOM | 970 | CB | LYS | 136 | −9.889 | 24.237 | 37.124 | 1.00 | 46.76 |
| ATOM | 971 | CG | LYS | 136 | −11.195 | 24.350 | 37.941 | 1.00 | 52.67 |
| ATOM | 972 | CD | LYS | 136 | −11.910 | 22.981 | 38.128 | 1.00 | 55.98 |
| ATOM | 973 | CE | LYS | 136 | −13.367 | 23.120 | 38.628 | 1.00 | 55.25 |
| ATOM | 974 | NZ | LYS | 136 | −14.106 | 21.817 | 38.719 | 1.00 | 51.28 |
| ATOM | 975 | C | LYS | 136 | −8.862 | 26.306 | 38.069 | 1.00 | 44.85 |
| ATOM | 976 | O | LYS | 136 | −9.730 | 26.894 | 38.717 | 1.00 | 45.87 |
| ATOM | 977 | N | HIS | 137 | −7.586 | 26.273 | 38.444 | 1.00 | 44.25 |
| ATOM | 978 | CA | HIS | 137 | −7.149 | 26.937 | 39.670 | 1.00 | 43.21 |
| ATOM | 979 | CB | HIS | 137 | −6.434 | 25.937 | 40.585 | 1.00 | 44.13 |
| ATOM | 980 | CG | HIS | 137 | −7.344 | 24.915 | 41.199 | 1.00 | 45.24 |
| ATOM | 981 | CD2 | HIS | 137 | −7.676 | 24.680 | 42.492 | 1.00 | 45.35 |
| ATOM | 982 | ND1 | HIS | 137 | −8.042 | 23.991 | 40.452 | 1.00 | 45.45 |
| ATOM | 983 | CE1 | HIS | 137 | −8.764 | 23.231 | 41.257 | 1.00 | 45.40 |
| ATOM | 984 | NE2 | HIS | 137 | −8.560 | 23.629 | 42.500 | 1.00 | 44.34 |
| ATOM | 985 | C | HIS | 137 | −6.242 | 28.132 | 39.400 | 1.00 | 41.96 |
| ATOM | 986 | O | HIS | 137 | −5.592 | 28.649 | 40.307 | 1.00 | 40.24 |
| ATOM | 987 | N | GLN | 38 | −6.217 | 28.577 | 38.151 | 1.00 | 42.87 |
| ATOM | 988 | CA | GLN | 138 | −5.390 | 29.706 | 37.766 | 1.00 | 44.93 |
| ATOM | 989 | CB | GLN | 138 | −5.949 | 30.993 | 38.373 | 1.00 | 47.58 |
| ATOM | 990 | CG | GLN | 138 | −7.258 | 31.448 | 37.749 | 1.00 | 51.96 |
| ATOM | 991 | CD | GLN | 138 | −7.416 | 32.966 | 37.766 | 1.00 | 55.20 |
| ATOM | 992 | OE1 | GLN | 138 | −6.680 | 33.698 | 37.088 | 1.00 | 56.05 |
| ATOM | 993 | NE2 | GLN | 138 | −8.375 | 33.445 | 38.546 | 1.00 | 55.44 |
| ATOM | 994 | C | GLN | 138 | −3.921 | 29.537 | 38.162 | 1.00 | 44.67 |
| ATOM | 995 | O | GLN | 138 | −3.316 | 30.437 | 38.747 | 1.00 | 45.78 |
| ATOM | 996 | N | MET | 139 | −3.350 | 28.383 | 37.836 | 1.00 | 41.86 |
| ATOM | 997 | CA | MET | 139 | −1.951 | 28.109 | 38.138 | 1.00 | 38.60 |
| ATOM | 998 | CB | MET | 139 | −1.846 | 27.062 | 39.236 | 1.00 | 39.19 |
| ATOM | 999 | CG | MET | 139 | −2.048 | 27.660 | 40.604 | 1.00 | 41.24 |
| ATOM | 1000 | SD | MET | 139 | −0.859 | 28.992 | 40.852 | 1.00 | 47.65 |
| ATOM | 1001 | CE | MET | 139 | 0.308 | 28.217 | 42.007 | 1.00 | 44.32 |
| ATOM | 1002 | C | MET | 139 | −1.232 | 27.653 | 36.881 | 1.00 | 36.60 |
| ATOM | 1003 | O | MET | 139 | −0.316 | 26.823 | 36.910 | 1.00 | 35.29 |
| ATOM | 1004 | N | LYS | 140 | −1.659 | 28.237 | 35.771 | 1.00 | 34.23 |
| ATOM | 1005 | CA | LYS | 140 | −1.101 | 27.921 | 34.477 | 1.00 | 32.15 |
| ATOM | 1006 | CB | LYS | 140 | −2.198 | 28.062 | 33.417 | 1.00 | 31.04 |
| ATOM | 1007 | CG | LYS | 140 | −1.970 | 27.293 | 32.116 | 1.00 | 31.48 |
| ATOM | 1008 | CD | LYS | 140 | −2.184 | 25.780 | 32.275 | 1.00 | 32.43 |
| ATOM | 1009 | CE | LYS | 140 | −2.112 | 25.015 | 30.925 | 1.00 | 30.89 |
| ATOM | 1010 | NZ | LYS | 140 | −0.811 | 25.130 | 30.168 | 1.00 | 29.56 |
| ATOM | 1011 | C | LYS | 140 | 0.085 | 28.834 | 34.161 | 1.00 | 31.02 |
| ATOM | 1012 | O | LYS | 140 | 0.047 | 30.045 | 34.412 | 1.00 | 29.99 |
| ATOM | 1013 | N | HIS | 141 | 1.143 | 28.228 | 33.627 | 1.00 | 31.35 |
| ATOM | 1014 | CA | HIS | 141 | 2.353 | 28.940 | 33.244 | 1.00 | 30.03 |
| ATOM | 1015 | CB | HIS | 141 | 1.989 | 30.145 | 32.385 | 1.00 | 30.05 |
| ATOM | 1016 | CG | HIS | 141 | 1.001 | 29.836 | 31.305 | 1.00 | 31.15 |
| ATOM | 1017 | CD2 | HIS | 141 | −0.132 | 30.473 | 30.927 | 1.00 | 30.91 |
| ATOM | 1018 | ND1 | HIS | 141 | 1.148 | 28.769 | 30.448 | 1.00 | 33.49 |
| ATOM | 1019 | CE1 | HIS | 141 | 0.147 | 28.763 | 29.584 | 1.00 | 35.03 |
| ATOM | 1020 | NE2 | HIS | 141 | −0.643 | 29.787 | 29.853 | 1.00 | 32.67 |
| ATOM | 1021 | C | HIS | 141 | 3.138 | 29.396 | 34.460 | 1.00 | 29.17 |
| ATOM | 1022 | O | HIS | 141 | 4.211 | 29.983 | 34.341 | 1.00 | 28.17 |
| ATOM | 1023 | N | LYS | 142 | 2.601 | 29.108 | 35.635 | 1.00 | 28.81 |
| ATOM | 1024 | CA | LYS | 142 | 3.248 | 29.505 | 36.869 | 1.00 | 29.17 |
| ATOM | 1025 | CB | LYS | 142 | 2.317 | 29.240 | 38.065 | 1.00 | 33.65 |
| ATOM | 1026 | CG | LYS | 142 | 0.986 | 30.042 | 38.072 | 1.00 | 39.35 |
| ATOM | 1027 | CD | LYS | 142 | 1.194 | 31.561 | 38.214 | 1.00 | 42.74 |
| ATOM | 1028 | CE | LYS | 142 | −0.122 | 32.360 | 38.170 | 1.00 | 45.49 |
| ATOM | 1029 | NZ | LYS | 142 | 0.110 | 33.844 | 38.325 | 1.00 | 46.19 |
| ATOM | 1030 | C | LYS | 142 | 4.575 | 28.785 | 37.075 | 1.00 | 26.49 |
| ATOM | 1031 | O | LYS | 142 | 5.340 | 29.138 | 37.966 | 1.00 | 26.10 |
| ATOM | 1032 | N | LYS | 143 | 4.862 | 27.784 | 36.254 | 1.00 | 24.58 |
| ATOM | 1033 | CA | LYS | 143 | 6.106 | 27.042 | 36.416 | 1.00 | 22.67 |
| ATOM | 1034 | CB | LYS | 143 | 7.258 | 27.847 | 35.836 | 1.00 | 21.51 |
| ATOM | 1035 | CG | LYS | 143 | 8.533 | 27.071 | 35.737 | 1.00 | 22.59 |
| ATOM | 1036 | CD | LYS | 143 | 9.319 | 27.510 | 34.516 | 1.00 | 25.81 |
| ATOM | 1037 | CE | LYS | 143 | 10.455 | 26.542 | 34.240 | 1.00 | 28.01 |
| ATOM | 1038 | NZ | LYS | 143 | 11.140 | 26.828 | 32.959 | 1.00 | 27.25 |
| ATOM | 1039 | C | LYS | 143 | 6.383 | 26.732 | 37.896 | 1.00 | 22.14 |
| ATOM | 1040 | O | LYS | 143 | 7.133 | 27.459 | 38.556 | 1.00 | 21.99 |
| ATOM | 1041 | N | LEU | 144 | 5.766 | 25.655 | 38.401 | 1.00 | 20.81 |
| ATOM | 1042 | CA | LEU | 144 | 5.910 | 25.214 | 39.797 | 1.00 | 16.90 |
| ATOM | 1043 | CB | LEU | 144 | 4.577 | 25.351 | 40.567 | 1.00 | 16.78 |
| ATOM | 1044 | CG | LEU | 144 | 3.208 | 24.956 | 39.983 | 1.00 | 18.43 |
| ATOM | 1045 | CD1 | LEU | 144 | 2.148 | 24.915 | 41.074 | 1.00 | 17.60 |
| ATOM | 1046 | CD2 | LEU | 144 | 2.795 | 25.960 | 38.929 | 1.00 | 19.20 |
| ATOM | 1047 | C | LEU | 144 | 6.432 | 23.781 | 39.933 | 1.00 | 15.80 |
| ATOM | 1048 | O | LEU | 144 | 6.265 | 22.958 | 39.032 | 1.00 | 12.24 |
| ATOM | 1049 | N | PRO | 145 | 7.078 | 23.478 | 41.076 | 1.00 | 16.26 |
| ATOM | 1050 | CD | PRO | 145 | 7.227 | 24.446 | 42.172 | 1.00 | 15.64 |
| ATOM | 1051 | CA | PRO | 145 | 7.678 | 22.196 | 41.467 | 1.00 | 14.17 |
| ATOM | 1052 | CB | PRO | 145 | 8.079 | 22.427 | 42.923 | 1.00 | 18.10 |
| ATOM | 1053 | CG | PRO | 145 | 8.378 | 23.860 | 42.963 | 1.00 | 17.14 |
| ATOM | 1054 | C | PRO | 145 | 6.707 | 21.050 | 41.357 | 1.00 | 12.75 |
| ATOM | 1055 | O | PRO | 145 | 5.580 | 21.141 | 41.852 | 1.00 | 12.27 |
| ATOM | 1056 | N | LEU | 146 | 7.160 | 19.957 | 40.758 | 1.00 | 10.29 |
| ATOM | 1057 | CA | LEU | 146 | 6.290 | 18.804 | 40.560 | 1.00 | 11.21 |
| ATOM | 1058 | CB | LEU | 146 | 6.156 | 18.539 | 39.075 | 1.00 | 7.24 |
| ATOM | 1059 | CG | LEU | 146 | 5.160 | 17.439 | 38.824 | 1.00 | 3.01 |
| ATOM | 1060 | CD1 | LEU | 146 | 3.817 | 17.832 | 39.389 | 1.00 | 1.00 |
| ATOM | 1061 | CD2 | LEU | 146 | 5.083 | 17.215 | 37.342 | 1.00 | 3.06 |
| ATOM | 1062 | C | LEU | 146 | 6.696 | 17.502 | 41.233 | 1.00 | 12.36 |
| ATOM | 1063 | O | LEU | 146 | 7.629 | 16.851 | 40.790 | 1.00 | 15.11 |
| ATOM | 1064 | N | GLY | 147 | 5.972 | 17.086 | 42.262 | 1.00 | 14.72 |
| ATOM | 1065 | CA | GLY | 147 | 6.333 | 15.851 | 42.937 | 1.00 | 17.81 |
| ATOM | 1066 | C | GLY | 147 | 5.716 | 14.586 | 42.371 | 1.00 | 18.51 |
| ATOM | 1067 | O | GLY | 147 | 4.689 | 14.644 | 41.704 | 1.00 | 20.85 |
| ATOM | 1068 | N | PHE | 148 | 6.342 | 13.440 | 42.631 | 1.00 | 19.28 |
| ATOM | 1069 | CA | PHE | 148 | 5.825 | 12.167 | 42.142 | 1.00 | 20.55 |
| ATOM | 1070 | CB | PHE | 148 | 6.707 | 11.635 | 41.023 | 1.00 | 16.36 |
| ATOM | 1071 | CG | PHE | 148 | 6.593 | 12.409 | 39.759 | 1.00 | 17.72 |
| ATOM | 1072 | CD1 | PHE | 148 | 6.792 | 13.779 | 39.753 | 1.00 | 17.99 |
| ATOM | 1073 | CD2 | PHE | 148 | 6.298 | 11.769 | 38.560 | 1.00 | 21.10 |
| ATOM | 1074 | CE1 | PHE | 148 | 6.695 | 14.509 | 38.570 | 1.00 | 22.37 |
| ATOM | 1075 | CE2 | PHE | 148 | 6.198 | 12.494 | 37.362 | 1.00 | 22.82 |
| ATOM | 1076 | CZ | PHE | 148 | 6.398 | 13.864 | 37.366 | 1.00 | 21.67 |
| ATOM | 1077 | C | PHE | 148 | 5.712 | 11.104 | 43.222 | 1.00 | 22.75 |
| ATOM | 1078 | O | PHE | 148 | 6.691 | 10.783 | 43.885 | 1.00 | 24.66 |
| ATOM | 1079 | N | THR | 149 | 4.513 | 10.562 | 43.403 | 1.00 | 24.45 |
| ATOM | 1080 | CA | THR | 149 | 4.312 | 9.514 | 44.387 | 1.00 | 24.75 |
| ATOM | 1081 | CB | THR | 149 | 3.365 | 9.917 | 45.497 | 1.00 | 23.76 |
| ATOM | 1082 | OG1 | THR | 149 | 2.757 | 11.175 | 45.192 | 1.00 | 25.51 |
| ATOM | 1083 | CG2 | THR | 149 | 4.107 | 9.989 | 46.786 | 1.00 | 22.63 |
| ATOM | 1084 | C | THR | 149 | 3.705 | 8.306 | 43.715 | 1.00 | 27.38 |
| ATOM | 1085 | O | THR | 149 | 3.093 | 8.405 | 42.647 | 1.00 | 24.58 |
| ATOM | 1086 | N | PHE | 150 | 3.857 | 7.160 | 44.361 | 1.00 | 30.07 |
| ATOM | 1087 | CA | PHE | 150 | 3.327 | 5.936 | 43.811 | 1.00 | 32.54 |
| ATOM | 1088 | CB | PHE | 150 | 4.455 | 5.120 | 43.215 | 1.00 | 29.97 |
| ATOM | 1089 | CG | PHE | 150 | 5.172 | 5.820 | 42.119 | 1.00 | 27.55 |
| ATOM | 1090 | CD1 | PHE | 150 | 6.134 | 6.770 | 42.397 | 1.00 | 27.41 |
| ATOM | 1091 | CD2 | PHE | 150 | 4.850 | 5.561 | 40.798 | 1.00 | 27.56 |
| ATOM | 1092 | CE1 | PHE | 150 | 6.770 | 7.447 | 41.366 | 1.00 | 28.61 |
| ATOM | 1093 | CE2 | PHE | 150 | 5.481 | 6.231 | 39.762 | 1.00 | 26.86 |
| ATOM | 1094 | CZ | PHE | 150 | 6.437 | 7.177 | 40.045 | 1.00 | 27.37 |
| ATOM | 1095 | C | PHE | 150 | 2.561 | 5.093 | 44.808 | 1.00 | 35.78 |
| ATOM | 1096 | O | PHE | 150 | 3.095 | 4.695 | 45.845 | 1.00 | 36.93 |
| ATOM | 1097 | N | SER | 151 | 1.305 | 4.813 | 44.467 | 1.00 | 38.60 |
| ATOM | 1098 | CA | SER | 151 | 0.420 | 4.006 | 45.295 | 1.00 | 40.51 |
| ATOM | 1099 | CB | SER | 151 | −0.830 | 4.802 | 45.641 | 1.00 | 41.51 |
| ATOM | 1100 | OG | SER | 151 | −1.507 | 5.159 | 44.453 | 1.00 | 47.40 |
| ATOM | 1101 | C | SER | 151 | 0.038 | 2.736 | 44.533 | 1.00 | 41.75 |
| ATOM | 1102 | O | SER | 151 | 0.069 | 2.696 | 43.301 | 1.00 | 40.78 |
| ATOM | 1103 | N | PHE | 152 | −0.336 | 1.704 | 45.278 | 1.00 | 43.86 |
| ATOM | 1104 | CA | PHE | 152 | −0.684 | 0.421 | 44.687 | 1.00 | 45.76 |
| ATOM | 1105 | CB | PHE | 152 | 0.465 | −0.557 | 44.965 | 1.00 | 51.67 |
| ATOM | 1106 | CG | PHE | 152 | 0.429 | −1.808 | 44.133 | 1.00 | 57.82 |
| ATOM | 1107 | CD1 | PHE | 152 | 0.597 | −1.749 | 42.751 | 1.00 | 59.39 |
| ATOM | 1108 | CD2 | PHE | 152 | 0.256 | −3.056 | 44.739 | 1.00 | 60.37 |
| ATOM | 1109 | CE1 | PHE | 152 | 0.598 | −2.915 | 41.979 | 1.00 | 61.23 |
| ATOM | 1110 | CE2 | PHE | 152 | 0.254 | −4.232 | 43.978 | 1.00 | 61.76 |
| ATOM | 1111 | CZ | PHE | 152 | 0.426 | −4.161 | 42.593 | 1.00 | 61.46 |
| ATOM | 1112 | C | PHE | 152 | −2.007 | −0.134 | 45.238 | 1.00 | 43.74 |
| ATOM | 1113 | O | PHE | 152 | −2.137 | −0.382 | 46.437 | 1.00 | 43.01 |
| ATOM | 1114 | N | PRO | 153 | −3.005 | −0.322 | 44.359 | 1.00 | 40.65 |
| ATOM | 1115 | CD | PRO | 153 | −2.993 | 0.179 | 42.979 | 1.00 | 39.35 |
| ATOM | 1116 | CA | PRO | 153 | −4.330 | −0.844 | 44.685 | 1.00 | 38.88 |
| ATOM | 1117 | CB | PRO | 153 | −5.045 | −0.803 | 43.352 | 1.00 | 36.16 |
| ATOM | 1118 | CG | PRO | 153 | −4.454 | 0.359 | 42.711 | 1.00 | 37.38 |
| ATOM | 1119 | C | PRO | 153 | −4.235 | −2.255 | 45.192 | 1.00 | 41.30 |
| ATOM | 1120 | O | PRO | 153 | −3.481 | −3.057 | 44.657 | 1.00 | 42.17 |
| ATOM | 1121 | N | VAL | 154 | −5.013 | −2.565 | 46.215 | 1.00 | 45.30 |
| ATOM | 1122 | CA | VAL | 154 | −5.016 | −3.905 | 46.767 | 1.00 | 49.50 |
| ATOM | 1123 | CB | VAL | 154 | −4.124 | −3.989 | 47.990 | 1.00 | 45.75 |
| ATOM | 1124 | CG1 | VAL | 154 | −4.297 | −5.331 | 48.638 | 1.00 | 45.79 |
| ATOM | 1125 | CG2 | VAL | 154 | −2.684 | −3.772 | 47.594 | 1.00 | 44.88 |
| ATOM | 1126 | C | VAL | 154 | −6.432 | −4.268 | 47.181 | 1.00 | 55.51 |
| ATOM | 1127 | O | VAL | 154 | −6.963 | −3.683 | 48.119 | 1.00 | 58.30 |
| ATOM | 1128 | N | ARG | 155 | −7.042 | −5.232 | 46.495 | 1.00 | 61.06 |
| ATOM | 1129 | CA | ARG | 155 | −8.413 | −5.643 | 46.812 | 1.00 | 67.71 |
| ATOM | 1130 | CB | ARG | 155 | −8.812 | −6.847 | 45.956 | 1.00 | 71.43 |
| ATOM | 1131 | CG | ARG | 155 | −9.033 | −6.501 | 44.501 | 1.00 | 76.11 |
| ATOM | 1132 | CD | ARG | 155 | −9.094 | −7.736 | 43.621 | 1.00 | 78.73 |
| ATOM | 1133 | NE | ARG | 155 | −9.292 | −7.352 | 42.226 | 1.00 | 81.59 |
| ATOM | 1134 | CZ | ARG | 155 | −9.138 | −8.168 | 41.190 | 1.00 | 82.83 |
| ATOM | 1135 | NH1 | ARG | 155 | −8.778 | −9.432 | 41.386 | 1.00 | 83.55 |
| ATOM | 1136 | NH2 | ARG | 155 | −9.340 | −7.717 | 39.956 | 1.00 | 82.01 |
| ATOM | 1137 | C | ARG | 155 | −8.639 | −5.965 | 48.291 | 1.00 | 70.15 |
| ATOM | 1138 | O | ARG | 155 | −7.689 | −6.255 | 49.022 | 1.00 | 71.24 |
| ATOM | 1139 | N | HIS | 156 | −9.903 | −5.923 | 48.720 | 1.00 | 71.23 |
| ATOM | 1140 | CA | HIS | 156 | −10.265 | −6.184 | 50.117 | 1.00 | 72.30 |
| ATOM | 1141 | CB | HIS | 156 | −11.724 | −5.769 | 50.365 | 1.00 | 73.82 |
| ATOM | 1142 | CG | HIS | 156 | −12.049 | −5.506 | 51.808 | 1.00 | 76.32 |
| ATOM | 1143 | CD2 | HIS | 156 | −11.335 | −5.722 | 52.941 | 1.00 | 76.70 |
| ATOM | 1144 | ND1 | HIS | 156 | −13.243 | −4.944 | 52.211 | 1.00 | 76.54 |
| ATOM | 1145 | CE1 | HIS | 156 | −13.251 | −4.823 | 53.527 | 1.00 | 76.16 |
| ATOM | 1146 | NE2 | HIS | 156 | −12.106 | −5.288 | 53.994 | 1.00 | 77.55 |
| ATOM | 1147 | C | HIS | 156 | −10.063 | −7.645 | 50.522 | 1.00 | 72.42 |
| ATOM | 1148 | O | HIS | 156 | −9.196 | −7.957 | 51.345 | 1.00 | 71.15 |
| ATOM | 1149 | N | ASN | 180 | 11.816 | 6.551 | 32.482 | 1.00 | 43.22 |
| ATOM | 1150 | CA | ASN | 180 | 11.492 | 7.278 | 33.706 | 1.00 | 42.73 |
| ATOM | 1151 | CB | ASN | 180 | 12.677 | 8.168 | 34.155 | 1.00 | 46.67 |
| ATOM | 1152 | CG | ASN | 180 | 13.189 | 9.094 | 33.052 | 1.00 | 50.13 |
| ATOM | 1153 | OD1 | ASN | 180 | 14.152 | 8.777 | 32.336 | 1.00 | 51.24 |
| ATOM | 1154 | ND2 | ASN | 180 | 12.547 | 10.250 | 32.915 | 1.00 | 51.73 |
| ATOM | 1155 | C | ASN | 180 | 10.228 | 8.110 | 33.523 | 1.00 | 38.44 |
| ATOM | 1156 | O | ASN | 180 | 9.941 | 8.600 | 32.431 | 1.00 | 36.40 |
| ATOM | 1157 | N | VAL | 181 | 9.473 | 8.257 | 34.603 | 1.00 | 34.02 |
| ATOM | 1158 | CA | VAL | 181 | 8.218 | 8.995 | 34.577 | 1.00 | 31.37 |
| ATOM | 1159 | CB | VAL | 181 | 7.498 | 8.874 | 35.957 | 1.00 | 34.84 |
| ATOM | 1160 | CG1 | VAL | 181 | 6.091 | 9.484 | 35.909 | 1.00 | 32.59 |
| ATOM | 1161 | CG2 | VAL | 181 | 7.414 | 7.405 | 36.353 | 1.00 | 38.00 |
| ATOM | 1162 | C | VAL | 181 | 8.426 | 10.458 | 34.221 | 1.00 | 26.36 |
| ATOM | 1163 | O | VAL | 181 | 7.882 | 10.964 | 33.237 | 1.00 | 23.28 |
| ATOM | 1164 | N | VAL | 182 | 9.228 | 11.131 | 35.030 | 1.00 | 23.56 |
| ATOM | 1165 | CA | VAL | 182 | 9.518 | 12.538 | 34.826 | 1.00 | 18.23 |
| ATOM | 1166 | CB | VAL | 182 | 10.702 | 12.958 | 35.716 | 1.00 | 14.26 |
| ATOM | 1167 | CG1 | VAL | 182 | 11.905 | 12.084 | 35.426 | 1.00 | 14.73 |
| ATOM | 1168 | CG2 | VAL | 182 | 11.001 | 14.403 | 35.508 | 1.00 | 11.08 |
| ATOM | 1169 | C | VAL | 182 | 9.773 | 12.882 | 33.352 | 1.00 | 15.36 |
| ATOM | 1170 | O | VAL | 182 | 9.330 | 13.924 | 32.875 | 1.00 | 15.32 |
| ATOM | 1171 | N | GLY | 183 | 10.467 | 12.009 | 32.632 | 1.00 | 13.34 |
| ATOM | 1172 | CA | GLY | 183 | 10.713 | 12.267 | 31.228 | 1.00 | 12.56 |
| ATOM | 1173 | C | GLY | 183 | 9.458 | 12.098 | 30.382 | 1.00 | 13.06 |
| ATOM | 1174 | O | GLY | 183 | 9.104 | 12.978 | 29.601 | 1.00 | 12.05 |
| ATOM | 1175 | N | LEU | 184 | 8.772 | 10.971 | 30.540 | 1.00 | 15.78 |
| ATOM | 1176 | CA | LEU | 184 | 7.549 | 10.708 | 29.777 | 1.00 | 15.21 |
| ATOM | 1177 | CB | LEU | 184 | 6.858 | 9.435 | 30.295 | 1.00 | 16.78 |
| ATOM | 1178 | CG | LEU | 184 | 7.613 | 8.108 | 30.075 | 1.00 | 15.45 |
| ATOM | 1179 | CD1 | LEU | 184 | 7.037 | 7.023 | 30.951 | 1.00 | 10.71 |
| ATOM | 1180 | CD2 | LEU | 184 | 7.548 | 7.708 | 28.608 | 1.00 | 16.62 |
| ATOM | 1181 | C | LEU | 184 | 6.601 | 11.894 | 29.863 | 1.00 | 13.07 |
| ATOM | 1182 | O | LEU | 184 | 6.041 | 12.311 | 28.855 | 1.00 | 13.90 |
| ATOM | 1183 | N | LEU | 185 | 6.430 | 12.436 | 31.064 | 1.00 | 11.99 |
| ATOM | 1184 | CA | LEU | 185 | 5.571 | 13.600 | 31.250 | 1.00 | 12.43 |
| ATOM | 1185 | CB | LEU | 185 | 5.524 | 13.997 | 32.729 | 1.00 | 13.27 |
| ATOM | 1186 | CG | LEU | 185 | 4.630 | 15.191 | 33.080 | 1.00 | 11.52 |
| ATOM | 1187 | CD1 | LEU | 185 | 3.256 | 14.936 | 32.515 | 1.00 | 10.60 |
| ATOM | 1188 | CD2 | LEU | 185 | 4.553 | 15.395 | 34.600 | 1.00 | 12.16 |
| ATOM | 1189 | C | LEU | 185 | 6.077 | 14.788 | 30.419 | 1.00 | 12.48 |
| ATOM | 1190 | O | LEU | 185 | 5.289 | 15.488 | 29.784 | 1.00 | 9.22 |
| ATOM | 1191 | N | ARG | 186 | 7.388 | 15.020 | 30.428 | 1.00 | 13.24 |
| ATOM | 1192 | CA | ARG | 186 | 7.946 | 16.123 | 29.661 | 1.00 | 14.83 |
| ATOM | 1193 | CB | ARG | 186 | 9.478 | 16.135 | 29.727 | 1.00 | 14.69 |
| ATOM | 1194 | CG | ARG | 186 | 10.112 | 17.274 | 30.526 | 1.00 | 18.47 |
| ATOM | 1195 | CD | ARG | 186 | 11.633 | 17.063 | 30.663 | 1.00 | 25.71 |
| ATOM | 1196 | NE | ARG | 186 | 12.325 | 18.069 | 31.484 | 1.00 | 37.62 |
| ATOM | 1197 | CZ | ARG | 186 | 12.048 | 18.357 | 32.764 | 1.00 | 42.54 |
| ATOM | 1198 | NH1 | ARG | 186 | 11.070 | 17.721 | 33.407 | 1.00 | 43.86 |
| ATOM | 1199 | NH2 | ARG | 186 | 12.762 | 19.277 | 33.414 | 1.00 | 39.97 |
| ATOM | 1200 | C | ARG | 186 | 7.510 | 15.968 | 28.220 | 1.00 | 16.38 |
| ATOM | 1201 | O | ARG | 186 | 6.857 | 16.851 | 27.673 | 1.00 | 17.00 |
| ATOM | 1202 | N | ASP | 187 | 7.850 | 14.832 | 27.616 | 1.00 | 19.34 |
| ATOM | 1203 | CA | ASP | 187 | 7.519 | 14.579 | 26.214 | 1.00 | 24.04 |
| ATOM | 1204 | CB | ASP | 187 | 7.799 | 13.123 | 25.822 | 1.00 | 30.35 |
| ATOM | 1205 | CG | ASP | 187 | 9.226 | 12.696 | 26.123 | 1.00 | 37.33 |
| ATOM | 1206 | OD1 | ASP | 187 | 9.479 | 12.216 | 27.251 | 1.00 | 40.99 |
| ATOM | 1207 | OD2 | ASP | 187 | 10.096 | 12.845 | 25.234 | 1.00 | 40.65 |
| ATOM | 1208 | C | ASP | 187 | 6.069 | 14.889 | 25.912 | 1.00 | 23.78 |
| ATOM | 1209 | O | ASP | 187 | 5.756 | 15.541 | 24.909 | 1.00 | 25.37 |
| ATOM | 1210 | N | ALA | 188 | 5.185 | 14.413 | 26.780 | 1.00 | 20.98 |
| ATOM | 1211 | CA | ALA | 188 | 3.761 | 14.634 | 26.603 | 1.00 | 17.11 |
| ATOM | 1212 | CB | ALA | 188 | 2.996 | 13.943 | 27.722 | 1.00 | 19.70 |
| ATOM | 1213 | C | ALA | 188 | 3.475 | 16.130 | 26.600 | 1.00 | 14.48 |
| ATOM | 1214 | O | ALA | 188 | 2.911 | 16.660 | 25.646 | 1.00 | 11.69 |
| ATOM | 1215 | N | ILE | 189 | 3.873 | 16.801 | 27.677 | 1.00 | 13.32 |
| ATOM | 1216 | CA | ILE | 189 | 3.682 | 18.239 | 27.817 | 1.00 | 13.84 |
| ATOM | 1217 | CB | ILE | 189 | 4.422 | 18.754 | 29.056 | 1.00 | 12.34 |
| ATOM | 1218 | CG2 | ILE | 189 | 4.368 | 20.266 | 29.118 | 1.00 | 13.98 |
| ATOM | 1219 | CG1 | ILE | 189 | 3.776 | 18.153 | 30.302 | 1.00 | 14.10 |
| ATOM | 1220 | CD1 | ILE | 189 | 4.455 | 18.530 | 31.595 | 1.00 | 14.04 |
| ATOM | 1221 | C | ILE | 189 | 4.223 | 18.928 | 26.575 | 1.00 | 15.60 |
| ATOM | 1222 | O | ILE | 189 | 3.634 | 19.888 | 26.058 | 1.00 | 14.87 |
| ATOM | 1223 | N | LYS | 190 | 5.351 | 18.408 | 26.103 | 1.00 | 16.13 |
| ATOM | 1224 | CA | LYS | 190 | 6.010 | 18.913 | 24.918 | 1.00 | 16.34 |
| ATOM | 1225 | CB | LYS | 190 | 7.361 | 18.211 | 24.737 | 1.00 | 18.43 |
| ATOM | 1226 | CG | LYS | 190 | 8.503 | 19.081 | 24.175 | 1.00 | 24.32 |
| ATOM | 1227 | CD | LYS | 190 | 8.539 | 19.154 | 22.631 | 1.00 | 28.76 |
| ATOM | 1228 | CE | LYS | 190 | 9.830 | 19.841 | 22.125 | 1.00 | 30.07 |
| ATOM | 1229 | NZ | LYS | 190 | 10.060 | 19.788 | 20.642 | 1.00 | 27.01 |
| ATOM | 1230 | C | LYS | 190 | 5.101 | 18.652 | 23.718 | 1.00 | 16.41 |
| ATOM | 1231 | O | LYS | 190 | 4.786 | 19.575 | 22.981 | 1.00 | 17.80 |
| ATOM | 1232 | N | ARG | 191 | 4.656 | 17.413 | 23.529 | 1.00 | 14.92 |
| ATOM | 1233 | CA | ARG | 191 | 3.798 | 17.107 | 22.386 | 1.00 | 15.62 |
| ATOM | 1234 | CB | ARG | 191 | 3.241 | 15.684 | 22.491 | 1.00 | 19.10 |
| ATOM | 1235 | CG | ARG | 191 | 4.071 | 14.622 | 21.775 | 1.00 | 20.57 |
| ATOM | 1236 | CD | ARG | 191 | 3.634 | 13.221 | 22.156 | 1.00 | 19.26 |
| ATOM | 1237 | NE | ARG | 191 | 3.950 | 12.925 | 23.547 | 1.00 | 23.45 |
| ATOM | 1238 | CZ | ARG | 191 | 3.732 | 11.747 | 24.119 | 1.00 | 28.59 |
| ATOM | 1239 | NH1 | ARG | 191 | 3.194 | 10.767 | 23.406 | 1.00 | 32.19 |
| ATOM | 1240 | NH2 | ARG | 191 | 4.062 | 11.537 | 25.391 | 1.00 | 29.84 |
| ATOM | 1241 | C | ARG | 191 | 2.652 | 18.086 | 22.207 | 1.00 | 15.44 |
| ATOM | 1242 | O | ARG | 191 | 2.383 | 18.513 | 21.098 | 1.00 | 15.17 |
| ATOM | 1243 | N | ARG | 192 | 1.980 | 18.441 | 23.295 | 1.00 | 17.09 |
| ATOM | 1244 | CA | ARG | 192 | 0.853 | 19.372 | 23.253 | 1.00 | 19.02 |
| ATOM | 1245 | CB | ARG | 192 | 0.588 | 19.885 | 24.647 | 1.00 | 17.94 |
| ATOM | 1246 | CG | ARG | 192 | 0.579 | 18.785 | 25.635 | 1.00 | 20.35 |
| ATOM | 1247 | CD | ARG | 192 | −0.812 | 18.328 | 25.855 | 1.00 | 22.03 |
| ATOM | 1248 | NE | ARG | 192 | −1.565 | 19.332 | 26.586 | 1.00 | 27.30 |
| ATOM | 1249 | CZ | ARG | 192 | −2.824 | 19.164 | 26.954 | 1.00 | 32.13 |
| ATOM | 1250 | NH1 | ARG | 192 | −3.437 | 18.028 | 26.639 | 1.00 | 34.51 |
| ATOM | 1251 | NH2 | ARG | 192 | −3.465 | 20.115 | 27.631 | 1.00 | 33.64 |
| ATOM | 1252 | C | ARG | 192 | 1.010 | 20.572 | 22.321 | 1.00 | 22.21 |
| ATOM | 1253 | O | ARG | 192 | 0.017 | 21.184 | 21.937 | 1.00 | 24.03 |
| ATOM | 1254 | N | GLY | 193 | 2.245 | 20.923 | 21.975 | 1.00 | 24.28 |
| ATOM | 1255 | CA | GLY | 193 | 2.472 | 22.052 | 21.088 | 1.00 | 25.59 |
| ATOM | 1256 | C | GLY | 193 | 2.351 | 23.417 | 21.750 | 1.00 | 27.55 |
| ATOM | 1257 | O | GLY | 193 | 2.734 | 24.437 | 21.163 | 1.00 | 26.53 |
| ATOM | 1258 | N | ASP | 194 | 1.836 | 23.434 | 22.981 | 1.00 | 28.09 |
| ATOM | 1259 | CA | ASP | 194 | 1.634 | 24.678 | 23.725 | 1.00 | 28.74 |
| ATOM | 1260 | CB | ASP | 194 | 0.349 | 24.597 | 24.548 | 1.00 | 32.11 |
| ATOM | 1261 | CG | ASP | 194 | −0.873 | 24.329 | 23.692 | 1.00 | 36.60 |
| ATOM | 1262 | OD1 | ASP | 194 | −1.053 | 25.025 | 22.668 | 1.00 | 38.48 |
| ATOM | 1263 | OD2 | ASP | 194 | −1.659 | 23.424 | 24.046 | 1.00 | 40.23 |
| ATOM | 1264 | C | ASP | 194 | 2.774 | 25.089 | 24.641 | 1.00 | 27.04 |
| ATOM | 1265 | O | ASP | 194 | 3.815 | 24.439 | 24.689 | 1.00 | 26.55 |
| ATOM | 1266 | N | PHE | 195 | 2.565 | 26.181 | 25.370 | 1.00 | 25.47 |
| ATOM | 1267 | CA | PHE | 195 | 3.582 | 26.691 | 26.274 | 1.00 | 25.41 |
| ATOM | 1268 | CB | PHE | 195 | 3.083 | 27.932 | 27.016 | 1.00 | 27.05 |
| ATOM | 1269 | CG | PHE | 195 | 3.156 | 29.192 | 26.201 | 1.00 | 28.43 |
| ATOM | 1270 | CD1 | PHE | 195 | 2.032 | 29.686 | 25.550 | 1.00 | 31.56 |
| ATOM | 1271 | CD2 | PHE | 195 | 4.353 | 29.880 | 26.067 | 1.00 | 29.11 |
| ATOM | 1272 | CE1 | PHE | 195 | 2.097 | 30.852 | 24.771 | 1.00 | 30.45 |
| ATOM | 1273 | CE2 | PHE | 195 | 4.426 | 31.046 | 25.290 | 1.00 | 30.62 |
| ATOM | 1274 | CZ | PHE | 195 | 3.294 | 31.528 | 24.644 | 1.00 | 29.47 |
| ATOM | 1275 | C | PHE | 195 | 4.024 | 25.642 | 27.267 | 1.00 | 24.15 |
| ATOM | 1276 | O | PHE | 195 | 3.214 | 25.083 | 28.000 | 1.00 | 25.61 |
| ATOM | 1277 | N | GLU | 196 | 5.324 | 25.385 | 27.280 | 1.00 | 22.49 |
| ATOM | 1278 | CA | GLU | 196 | 5.897 | 24.394 | 28.166 | 1.00 | 23.12 |
| ATOM | 1279 | CB | GLU | 196 | 7.117 | 23.754 | 27.499 | 1.00 | 21.72 |
| ATOM | 1280 | CG | GLU | 196 | 6.942 | 23.418 | 26.020 | 1.00 | 22.22 |
| ATOM | 1281 | CD | GLU | 196 | 8.121 | 22.629 | 25.477 | 1.00 | 24.60 |
| ATOM | 1282 | OE1 | GLU | 196 | 8.336 | 22.601 | 24.241 | 1.00 | 23.54 |
| ATOM | 1283 | OE2 | GLU | 196 | 8.839 | 22.026 | 26.301 | 1.00 | 26.49 |
| ATOM | 1284 | C | GLU | 196 | 6.314 | 25.066 | 29.466 | 1.00 | 24.25 |
| ATOM | 1285 | O | GLU | 196 | 7.467 | 24.966 | 29.882 | 1.00 | 26.05 |
| ATOM | 1286 | N | MET | 197 | 5.376 | 25.729 | 30.126 | 1.00 | 25.12 |
| ATOM | 1287 | CA | MET | 197 | 5.711 | 26.444 | 31.352 | 1.00 | 27.52 |
| ATOM | 1288 | CB | MET | 197 | 5.546 | 27.942 | 31.096 | 1.00 | 29.51 |
| ATOM | 1289 | CG | MET | 197 | 6.758 | 28.782 | 31.466 | 1.00 | 33.61 |
| ATOM | 1290 | SD | MET | 197 | 7.208 | 29.992 | 30.181 | 1.00 | 35.72 |
| ATOM | 1291 | CE | MET | 197 | 5.967 | 31.256 | 30.466 | 1.00 | 37.45 |
| ATOM | 1292 | C | MET | 197 | 4.906 | 26.045 | 32.583 | 1.00 | 27.47 |
| ATOM | 1293 | O | MET | 197 | 4.921 | 26.749 | 33.597 | 1.00 | 25.63 |
| ATOM | 1294 | N | ASP | 198 | 4.230 | 24.903 | 32.502 | 1.00 | 27.57 |
| ATOM | 1295 | CA | ASP | 198 | 3.384 | 24.430 | 33.598 | 1.00 | 26.12 |
| ATOM | 1296 | CB | ASP | 198 | 2.462 | 23.298 | 33.110 | 1.00 | 29.89 |
| ATOM | 1297 | CG | ASP | 198 | 1.326 | 23.796 | 32.232 | 1.00 | 31.76 |
| ATOM | 1298 | OD1 | ASP | 198 | 0.736 | 24.840 | 32.590 | 1.00 | 30.59 |
| ATOM | 1299 | OD2 | ASP | 198 | 1.023 | 23.135 | 31.203 | 1.00 | 32.71 |
| ATOM | 1300 | C | ASP | 198 | 4.110 | 23.959 | 34.853 | 1.00 | 22.26 |
| ATOM | 1301 | O | ASP | 198 | 3.960 | 24.551 | 35.923 | 1.00 | 18.00 |
| ATOM | 1302 | N | VAL | 199 | 4.873 | 22.878 | 34.717 | 1.00 | 19.81 |
| ATOM | 1303 | CA | VAL | 199 | 5.605 | 22.301 | 35.841 | 1.00 | 18.78 |
| ATOM | 1304 | CB | VAL | 199 | 5.133 | 20.852 | 36.115 | 1.00 | 16.48 |
| ATOM | 1305 | CG1 | VAL | 199 | 3.736 | 20.859 | 36.696 | 1.00 | 19.07 |
| ATOM | 1306 | CG2 | VAL | 199 | 5.150 | 20.042 | 34.823 | 1.00 | 10.86 |
| ATOM | 1307 | C | VAL | 199 | 7.121 | 22.267 | 35.648 | 1.00 | 20.19 |
| ATOM | 1308 | O | VAL | 199 | 7.665 | 22.752 | 34.655 | 1.00 | 21.16 |
| ATOM | 1309 | N | VAL | 200 | 7.798 | 21.695 | 36.629 | 1.00 | 20.40 |
| ATOM | 1310 | CA | VAL | 200 | 9.237 | 21.547 | 36.594 | 1.00 | 22.39 |
| ATOM | 1311 | CB | VAL | 200 | 9.975 | 22.834 | 37.007 | 1.00 | 24.84 |
| ATOM | 1312 | CG1 | VAL | 200 | 9.331 | 23.406 | 38.255 | 1.00 | 31.58 |
| ATOM | 1313 | CG2 | VAL | 200 | 11.465 | 22.539 | 37.266 | 1.00 | 21.54 |
| ATOM | 1314 | C | VAL | 200 | 9.502 | 20.457 | 37.598 | 1.00 | 23.06 |
| ATOM | 1315 | O | VAL | 200 | 9.039 | 20.501 | 38.755 | 1.00 | 22.26 |
| ATOM | 1316 | N | ALA | 201 | 10.229 | 19.460 | 37.120 | 1.00 | 23.03 |
| ATOM | 1317 | CA | ALA | 201 | 10.569 | 18.300 | 37.907 | 1.00 | 22.74 |
| ATOM | 1318 | CB | ALA | 201 | 11.460 | 17.418 | 37.112 | 1.00 | 23.66 |
| ATOM | 1319 | C | ALA | 201 | 11.236 | 18.646 | 39.209 | 1.00 | 24.47 |
| ATOM | 1320 | O | ALA | 201 | 12.045 | 19.564 | 39.285 | 1.00 | 27.55 |
| ATOM | 1321 | N | MET | 202 | 10.872 | 17.914 | 40.244 | 1.00 | 25.96 |
| ATOM | 1322 | CA | MET | 202 | 11.479 | 18.106 | 41.547 | 1.00 | 27.52 |
| ATOM | 1323 | CB | MET | 202 | 10.720 | 19.124 | 42.386 | 1.00 | 27.45 |
| ATOM | 1324 | CG | MET | 202 | 11.516 | 19.580 | 43.597 | 1.00 | 27.56 |
| ATOM | 1325 | SD | MET | 202 | 11.967 | 18.244 | 44.740 | 1.00 | 28.85 |
| ATOM | 1326 | CE | MET | 202 | 10.732 | 18.486 | 46.045 | 1.00 | 23.74 |
| ATOM | 1327 | C | MET | 202 | 11.436 | 16.752 | 42.219 | 1.00 | 28.79 |
| ATOM | 1328 | O | MET | 202 | 10.377 | 16.290 | 42.653 | 1.00 | 25.51 |
| ATOM | 1329 | N | VAL | 203 | 12.600 | 16.118 | 42.293 | 1.00 | 29.76 |
| ATOM | 1330 | CA | VAL | 203 | 12.695 | 14.802 | 42.883 | 1.00 | 28.97 |
| ATOM | 1331 | CB | VAL | 203 | 12.943 | 13.727 | 41.813 | 1.00 | 25.86 |
| ATOM | 1332 | CGI | VAL | 203 | 11.936 | 13.870 | 40.681 | 1.00 | 22.02 |
| ATOM | 1333 | CG2 | VAL | 203 | 14.361 | 13.831 | 41.310 | 1.00 | 23.30 |
| ATOM | 1334 | C | VAL | 203 | 13.815 | 14.713 | 43.890 | 1.00 | 31.36 |
| ATOM | 1335 | O | VAL | 203 | 13.934 | 13.713 | 44.585 | 1.00 | 34.93 |
| ATOM | 1336 | N | ASN | 204 | 14.638 | 15.745 | 43.987 | 1.00 | 32.12 |
| ATOM | 1337 | CA | ASN | 204 | 15.741 | 15.674 | 44.929 | 1.00 | 33.37 |
| ATOM | 1338 | CB | ASN | 204 | 16.667 | 16.867 | 44.736 | 1.00 | 36.19 |
| ATOM | 1339 | CG | ASN | 204 | 18.052 | 16.601 | 45.260 | 1.00 | 39.20 |
| ATOM | 1340 | OD1 | ASN | 204 | 18.847 | 15.905 | 44.621 | 1.00 | 41.71 |
| ATOM | 1341 | ND2 | ASN | 204 | 18.349 | 17.133 | 46.440 | 1.00 | 39.72 |
| ATOM | 1342 | C | ASN | 204 | 15.220 | 15.625 | 46.363 | 1.00 | 32.02 |
| ATOM | 1343 | O | ASN | 204 | 14.382 | 16.439 | 46.751 | 1.00 | 28.87 |
| ATOM | 1344 | N | ASP | 205 | 15.705 | 14.665 | 47.149 | 1.00 | 31.97 |
| ATOM | 1345 | CA | ASP | 205 | 15.245 | 14.538 | 48.541 | 1.00 | 33.94 |
| ATOM | 1346 | CB | ASP | 205 | 15.792 | 13.266 | 49.197 | 1.00 | 32.38 |
| ATOM | 1347 | CG | ASP | 205 | 15.163 | 12.017 | 48.642 | 1.00 | 31.18 |
| ATOM | 1348 | OD1 | ASP | 205 | 15.386 | 10.935 | 49.217 | 1.00 | 31.15 |
| ATOM | 1349 | OD2 | ASP | 205 | 14.450 | 12.118 | 47.625 | 1.00 | 28.42 |
| ATOM | 1350 | C | ASP | 205 | 15.626 | 15.722 | 49.414 | 1.00 | 33.64 |
| ATOM | 1351 | O | ASP | 205 | 14.909 | 16.080 | 50.356 | 1.00 | 33.83 |
| ATOM | 1352 | N | THR | 206 | 16.770 | 16.313 | 49.092 | 1.00 | 31.15 |
| ATOM | 1353 | CA | THR | 206 | 17.290 | 17.449 | 49.826 | 1.00 | 25.09 |
| ATOM | 1354 | CB | THR | 206 | 18.646 | 17.825 | 49.278 | 1.00 | 25.45 |
| ATOM | 1355 | OG1 | THR | 206 | 19.423 | 16.630 | 49.123 | 1.00 | 24.81 |
| ATOM | 1356 | CG2 | THR | 206 | 19.350 | 18.769 | 50.232 | 1.00 | 26.26 |
| ATOM | 1357 | C | THR | 206 | 16.347 | 18.634 | 49.734 | 1.00 | 20.16 |
| ATOM | 1358 | O | THR | 206 | 15.923 | 19.184 | 50.755 | 1.00 | 17.86 |
| ATOM | 1359 | N | VAL | 207 | 16.009 | 19.016 | 48.510 | 1.00 | 12.86 |
| ATOM | 1360 | CA | VAL | 207 | 15.106 | 20.133 | 48.308 | 1.00 | 9.27 |
| ATOM | 1361 | CB | VAL | 207 | 14.582 | 20.164 | 46.867 | 1.00 | 5.21 |
| ATOM | 1362 | CG1 | VAL | 207 | 13.555 | 21.243 | 46.720 | 1.00 | 1.26 |
| ATOM | 1363 | CG2 | VAL | 207 | 15.714 | 20.397 | 45.910 | 1.00 | 4.57 |
| ATOM | 1364 | C | VAL | 207 | 13.917 | 19.992 | 49.255 | 1.00 | 11.72 |
| ATOM | 1365 | O | VAL | 207 | 13.584 | 20.909 | 50.016 | 1.00 | 9.00 |
| ATOM | 1366 | N | ALA | 208 | 13.291 | 18.819 | 49.212 | 1.00 | 14.04 |
| ATOM | 1367 | CA | ALA | 208 | 12.122 | 18.523 | 50.041 | 1.00 | 14.67 |
| ATOM | 1368 | CB | ALA | 208 | 11.598 | 17.148 | 49.702 | 1.00 | 14.60 |
| ATOM | 1369 | C | ALA | 208 | 12.422 | 18.615 | 51.537 | 1.00 | 15.41 |
| ATOM | 1370 | O | ALA | 208 | 11.514 | 18.770 | 52.362 | 1.00 | 14.28 |
| ATOM | 1371 | N | THR | 209 | 13.699 | 18.498 | 51.879 | 1.00 | 13.94 |
| ATOM | 1372 | CA | THR | 209 | 14.123 | 18.591 | 53.261 | 1.00 | 13.05 |
| ATOM | 1373 | CB | THR | 209 | 15.567 | 18.237 | 53.423 | 1.00 | 11.66 |
| ATOM | 1374 | OG1 | THR | 209 | 15.887 | 17.177 | 52.525 | 1.00 | 12.70 |
| ATOM | 1375 | CG2 | THR | 209 | 15.833 | 17.807 | 54.846 | 1.00 | 7.92 |
| ATOM | 1376 | C | THR | 209 | 14.007 | 20.041 | 53.626 | 1.00 | 14.97 |
| ATOM | 1377 | O | THR | 209 | 13.554 | 20.401 | 54.714 | 1.00 | 14.80 |
| ATOM | 1378 | N | MET | 210 | 14.447 | 20.885 | 52.707 | 1.00 | 15.34 |
| ATOM | 1379 | CA | MET | 210 | 14.363 | 22.298 | 52.965 | 1.00 | 16.36 |
| ATOM | 1380 | CB | MET | 210 | 15.043 | 23.091 | 51.845 | 1.00 | 19.89 |
| ATOM | 1381 | CG | MET | 210 | 15.119 | 24.592 | 52.103 | 1.00 | 23.82 |
| ATOM | 1382 | SD | MET | 210 | 15.258 | 25.542 | 50.561 | 1.00 | 29.33 |
| ATOM | 1383 | CE | MET | 210 | 13.547 | 25.995 | 50.325 | 1.00 | 27.80 |
| ATOM | 1384 | C | MET | 210 | 12.864 | 22.592 | 53.031 | 1.00 | 14.33 |
| ATOM | 1385 | O | MET | 210 | 12.332 | 22.896 | 54.102 | 1.00 | 15.04 |
| ATOM | 1386 | N | ILE | 211 | 12.180 | 22.452 | 51.898 | 1.00 | 11.15 |
| ATOM | 1387 | CA | ILE | 211 | 10.743 | 22.708 | 51.831 | 1.00 | 9.09 |
| ATOM | 1388 | CB | ILE | 211 | 10.157 | 22.122 | 50.566 | 1.00 | 5.39 |
| ATOM | 1389 | CG2 | ILE | 211 | 8.748 | 22.693 | 50.337 | 1.00 | 3.22 |
| ATOM | 1390 | CG1 | ILE | 211 | 11.111 | 22.412 | 49.412 | 1.00 | 2.02 |
| ATOM | 1391 | CD1 | ILE | 211 | 10.580 | 22.065 | 48.067 | 1.00 | 1.00 |
| ATOM | 1392 | C | ILE | 211 | 9.987 | 22.129 | 53.022 | 1.00 | 10.92 |
| ATOM | 1393 | O | ILE | 211 | 9.117 | 22.781 | 53.605 | 1.00 | 7.92 |
| ATOM | 1394 | N | SER | 212 | 10.319 | 20.891 | 53.364 | 1.00 | 12.74 |
| ATOM | 1395 | CA | SER | 212 | 9.701 | 20.254 | 54.489 | 1.00 | 15.18 |
| ATOM | 1396 | CB | SER | 212 | 10.300 | 18.880 | 54.704 | 1.00 | 12.84 |
| ATOM | 1397 | OG | SER | 212 | 10.216 | 18.533 | 56.078 | 1.00 | 19.56 |
| ATOM | 1398 | C | SER | 212 | 9.918 | 21.101 | 55.736 | 1.00 | 19.90 |
| ATOM | 1399 | O | SER | 212 | 8.969 | 21.432 | 56.435 | 1.00 | 21.30 |
| ATOM | 1400 | N | CYS | 213 | 11.161 | 21.476 | 56.016 | 1.00 | 24.22 |
| ATOM | 1401 | CA | CYS | 213 | 11.432 | 22.259 | 57.219 | 1.00 | 28.52 |
| ATOM | 1402 | CB | CYS | 213 | 12.934 | 22.367 | 57.464 | 1.00 | 30.65 |
| ATOM | 1403 | SG | CYS | 213 | 13.713 | 20.766 | 57.805 | 1.00 | 39.09 |
| ATOM | 1404 | C | CYS | 213 | 10.822 | 23.637 | 57.168 | 1.00 | 29.40 |
| ATOM | 1405 | O | CYS | 213 | 10.366 | 24.150 | 58.186 | 1.00 | 30.64 |
| ATOM | 1406 | N | TYR | 214 | 10.816 | 24.229 | 55.981 | 1.00 | 29.50 |
| ATOM | 1407 | CA | TYR | 214 | 10.243 | 25.548 | 55.788 | 1.00 | 29.27 |
| ATOM | 1408 | CB | TYR | 214 | 10.168 | 25.846 | 54.292 | 1.00 | 31.33 |
| ATOM | 1409 | CG | TYR | 214 | 9.637 | 27.212 | 53.985 | 1.00 | 33.15 |
| ATOM | 1410 | CD1 | TYR | 214 | 10.182 | 28.328 | 54.594 | 1.00 | 36.28 |
| ATOM | 1411 | CE1 | TYR | 214 | 9.694 | 29.592 | 54.341 | 1.00 | 39.73 |
| ATOM | 1412 | CD2 | TYR | 214 | 8.582 | 27.390 | 53.100 | 1.00 | 35.23 |
| ATOM | 1413 | CE2 | TYR | 214 | 8.080 | 28.656 | 52.833 | 1.00 | 39.38 |
| ATOM | 1414 | CZ | TYR | 214 | 8.644 | 29.758 | 53.463 | 1.00 | 41.11 |
| ATOM | 1415 | OH | TYR | 214 | 8.168 | 31.034 | 53.241 | 1.00 | 43.33 |
| ATOM | 1416 | C | TYR | 214 | 8.848 | 25.649 | 56.429 | 1.00 | 28.57 |
| ATOM | 1417 | O | TYR | 214 | 8.561 | 26.578 | 57.185 | 1.00 | 27.99 |
| ATOM | 1418 | N | TYR | 215 | 7.986 | 24.685 | 56.136 | 1.00 | 27.91 |
| ATOM | 1419 | CA | TYR | 215 | 6.642 | 24.685 | 56.691 | 1.00 | 27.12 |
| ATOM | 1420 | CB | TYR | 215 | 5.922 | 23.403 | 56.309 | 1.00 | 21.95 |
| ATOM | 1421 | CG | TYR | 215 | 5.723 | 23.235 | 54.829 | 1.00 | 18.06 |
| ATOM | 1422 | CD1 | TYR | 215 | 6.064 | 22.048 | 54.197 | 1.00 | 17.25 |
| ATOM | 1423 | CE1 | TYR | 215 | 5.835 | 21.867 | 52.841 | 1.00 | 17.47 |
| ATOM | 1424 | CD2 | TYR | 215 | 5.152 | 24.246 | 54.065 | 1.00 | 16.51 |
| ATOM | 1425 | CE2 | TYR | 215 | 4.917 | 24.075 | 52.711 | 1.00 | 15.51 |
| ATOM | 1426 | CZ | TYR | 215 | 5.257 | 22.882 | 52.109 | 1.00 | 17.82 |
| ATOM | 1427 | OH | TYR | 215 | 4.979 | 22.681 | 50.785 | 1.00 | 20.98 |
| ATOM | 1428 | C | TYR | 215 | 6.658 | 24.810 | 58.201 | 1.00 | 30.19 |
| ATOM | 1429 | O | TYR | 215 | 5.780 | 25.438 | 58.778 | 1.00 | 31.10 |
| ATOM | 1430 | N | GLU | 216 | 7.640 | 24.197 | 58.850 | 1.00 | 35.15 |
| ATOM | 1431 | CA | GLU | 216 | 7.725 | 24.278 | 60.306 | 1.00 | 41.19 |
| ATOM | 1432 | CB | GLU | 216 | 8.560 | 23.132 | 60.876 | 1.00 | 44.10 |
| ATOM | 1433 | CG | GLU | 216 | 7.877 | 21.767 | 60.887 | 1.00 | 52.19 |
| ATOM | 1434 | CD | GLU | 216 | 6.579 | 21.749 | 61.685 | 1.00 | 54.93 |
| ATOM | 1435 | OE1 | GLU | 216 | 6.491 | 22.481 | 62.702 | 1.00 | 55.44 |
| ATOM | 1436 | OE2 | GLU | 216 | 5.658 | 20.988 | 61.296 | 1.00 | 56.26 |
| ATOM | 1437 | C | GLU | 216 | 8.369 | 25.591 | 60.707 | 1.00 | 43.33 |
| ATOM | 1438 | O | GLU | 216 | 7.787 | 26.385 | 61.449 | 1.00 | 44.64 |
| ATOM | 1439 | N | ASP | 217 | 9.583 | 25.802 | 60.209 | 1.00 | 44.35 |
| ATOM | 1440 | CA | ASP | 217 | 10.357 | 27.007 | 60.489 | 1.00 | 44.65 |
| ATOM | 1441 | CB | ASP | 217 | 11.734 | 26.623 | 61.033 | 1.00 | 47.71 |
| ATOM | 1442 | CG | ASP | 217 | 12.667 | 27.806 | 61.136 | 1.00 | 50.46 |
| ATOM | 1443 | OD1 | ASP | 217 | 13.252 | 28.205 | 60.106 | 1.00 | 51.95 |
| ATOM | 1444 | OD2 | ASP | 217 | 12.804 | 28.346 | 62.252 | 1.00 | 54.06 |
| ATOM | 1445 | C | ASP | 217 | 10.514 | 27.820 | 59.215 | 1.00 | 43.04 |
| ATOM | 1446 | O | ASP | 217 | 11.372 | 27.527 | 58.385 | 1.00 | 44.60 |
| ATOM | 1447 | N | HIS | 218 | 9.691 | 28.848 | 59.059 | 1.00 | 41.00 |
| ATOM | 1448 | CA | HIS | 218 | 9.750 | 29.671 | 57.862 | 1.00 | 39.42 |
| ATOM | 1449 | CB | HIS | 218 | 8.569 | 30.630 | 57.826 | 1.00 | 40.46 |
| ATOM | 1450 | CG | HIS | 218 | 7.261 | 29.960 | 58.083 | 1.00 | 44.54 |
| ATOM | 1451 | CD2 | HIS | 218 | 6.652 | 28.930 | 57.450 | 1.00 | 45.30 |
| ATOM | 1452 | ND1 | HIS | 218 | 6.449 | 30.290 | 59.147 | 1.00 | 47.09 |
| ATOM | 1453 | CE1 | HIS | 218 | 5.397 | 29.492 | 59.161 | 1.00 | 45.61 |
| ATOM | 1454 | NE2 | HIS | 218 | 5.497 | 28.657 | 58.142 | 1.00 | 46.44 |
| ATOM | 1455 | C | HIS | 218 | 11.036 | 30.452 | 57.759 | 1.00 | 37.69 |
| ATOM | 1456 | O | HIS | 218 | 11.120 | 31.381 | 56.974 | 1.00 | 37.21 |
| ATOM | 1457 | N | GLN | 219 | 12.041 | 30.076 | 58.537 | 1.00 | 37.38 |
| ATOM | 1458 | CA | GLN | 219 | 13.312 | 30.779 | 58.494 | 1.00 | 38.18 |
| ATOM | 1459 | CB | GLN | 219 | 13.727 | 31.186 | 59.910 | 1.00 | 41.72 |
| ATOM | 1460 | CG | GLN | 219 | 14.577 | 32.451 | 60.011 | 1.00 | 48.69 |
| ATOM | 1461 | CD | GLN | 219 | 13.836 | 33.718 | 59.546 | 1.00 | 55.14 |
| ATOM | 1462 | OE1 | GLN | 219 | 12.665 | 33.945 | 59.908 | 1.00 | 55.89 |
| ATOM | 1463 | NE2 | GLN | 219 | 14.523 | 34.555 | 58.751 | 1.00 | 55.41 |
| ATOM | 1464 | C | GLN | 219 | 14.348 | 29.846 | 57.886 | 1.00 | 36.85 |
| ATOM | 1465 | O | GLN | 219 | 15.508 | 30.200 | 57.735 | 1.00 | 37.28 |
| ATOM | 1466 | N | CYS | 220 | 13.912 | 28.647 | 57.535 | 1.00 | 36.02 |
| ATOM | 1467 | CA | CYS | 220 | 14.790 | 27.646 | 56.950 | 1.00 | 37.10 |
| ATOM | 1468 | CB | CYS | 220 | 14.103 | 26.286 | 57.043 | 1.00 | 38.40 |
| ATOM | 1469 | SG | CYS | 220 | 15.067 | 24.916 | 56.396 | 1.00 | 44.24 |
| ATOM | 1470 | C | CYS | 220 | 15.106 | 27.970 | 55.486 | 1.00 | 37.48 |
| ATOM | 1471 | O | CYS | 220 | 14.193 | 28.081 | 54.672 | 1.00 | 40.52 |
| ATOM | 1472 | N | GLU | 221 | 16.382 | 28.123 | 55.137 | 1.00 | 36.17 |
| ATOM | 1473 | CA | GLU | 221 | 16.742 | 28.428 | 53.746 | 1.00 | 35.58 |
| ATOM | 1474 | CB | GLU | 221 | 17.116 | 29.911 | 53.591 | 1.00 | 38.60 |
| ATOM | 1475 | CG | GLU | 221 | 15.921 | 30.878 | 53.645 | 1.00 | 42.48 |
| ATOM | 1476 | CD | GLU | 221 | 16.325 | 32.347 | 53.760 | 1.00 | 42.62 |
| ATOM | 1477 | OE1 | GLU | 221 | 17.120 | 32.815 | 52.909 | 1.00 | 42.89 |
| ATOM | 1478 | OE2 | GLU | 221 | 15.835 | 33.024 | 54.700 | 1.00 | 40.36 |
| ATOM | 1479 | C | GLU | 221 | 17.896 | 27.566 | 53.260 | 1.00 | 33.89 |
| ATOM | 1480 | O | GLU | 221 | 18.498 | 27.826 | 52.217 | 1.00 | 32.29 |
| ATOM | 1481 | N | VAL | 222 | 18.199 | 26.525 | 54.018 | 1.00 | 32.57 |
| ATOM | 1482 | CA | VAL | 222 | 19.286 | 25.654 | 53.645 | 1.00 | 31.01 |
| ATOM | 1483 | CB | VAL | 222 | 20.548 | 26.041 | 54.376 | 1.00 | 29.59 |
| ATOM | 1484 | CG1 | VAL | 222 | 21.673 | 25.102 | 53.995 | 1.00 | 29.07 |
| ATOM | 1485 | CG2 | VAL | 222 | 20.895 | 27.465 | 54.043 | 1.00 | 30.00 |
| ATOM | 1486 | C | VAL | 222 | 18.983 | 24.214 | 53.966 | 1.00 | 31.75 |
| ATOM | 1487 | O | VAL | 222 | 18.872 | 23.846 | 55.132 | 1.00 | 33.50 |
| ATOM | 1488 | N | GLY | 223 | 18.858 | 23.400 | 52.925 | 1.00 | 31.02 |
| ATOM | 1489 | CA | GLY | 223 | 18.575 | 21.994 | 53.119 | 1.00 | 28.49 |
| ATOM | 1490 | C | GLY | 223 | 19.847 | 21.184 | 53.026 | 1.00 | 26.21 |
| ATOM | 1491 | O | GLY | 223 | 20.757 | 21.528 | 52.267 | 1.00 | 25.39 |
| ATOM | 1492 | N | MET | 224 | 19.911 | 20.098 | 53.786 | 1.00 | 24.93 |
| ATOM | 1493 | CA | MET | 224 | 21.101 | 19.267 | 53.774 | 1.00 | 24.66 |
| ATOM | 1494 | CB | MET | 224 | 22.164 | 19.958 | 54.623 | 1.00 | 26.07 |
| ATOM | 1495 | CG | MET | 224 | 23.584 | 19.535 | 54.358 | 1.00 | 26.25 |
| ATOM | 1496 | SD | MET | 224 | 24.664 | 20.375 | 55.525 | 1.00 | 28.76 |
| ATOM | 1497 | CE | MET | 224 | 24.493 | 19.328 | 56.939 | 1.00 | 27.46 |
| ATOM | 1498 | C | MET | 224 | 20.867 | 17.819 | 54.253 | 1.00 | 23.62 |
| ATOM | 1499 | O | MET | 224 | 20.243 | 17.581 | 55.294 | 1.00 | 21.62 |
| ATOM | 1500 | N | ILE | 225 | 21.389 | 16.867 | 53.478 | 1.00 | 21.96 |
| ATOM | 1501 | CA | ILE | 225 | 21.265 | 15.434 | 53.764 | 1.00 | 21.80 |
| ATOM | 1502 | CB | ILE | 225 | 20.514 | 14.706 | 52.662 | 1.00 | 23.26 |
| ATOM | 1503 | CG2 | ILE | 225 | 20.389 | 13.242 | 53.026 | 1.00 | 22.57 |
| ATOM | 1504 | CG1 | ILE | 225 | 19.142 | 15.332 | 52.463 | 1.00 | 26.22 |
| ATOM | 1505 | CD1 | ILE | 225 | 18.270 | 15.229 | 53.688 | 1.00 | 30.06 |
| ATOM | 1506 | C | ILE | 225 | 22.595 | 14.702 | 53.904 | 1.00 | 21.76 |
| ATOM | 1507 | O | ILE | 225 | 23.204 | 14.299 | 52.909 | 1.00 | 20.84 |
| ATOM | 1508 | N | VAL | 226 | 23.008 | 14.492 | 55.146 | 1.00 | 22.14 |
| ATOM | 1509 | CA | VAL | 226 | 24.263 | 13.824 | 55.454 | 1.00 | 22.07 |
| ATOM | 1510 | CB | VAL | 226 | 25.031 | 14.613 | 56.514 | 1.00 | 22.20 |
| ATOM | 1511 | CG1 | VAL | 226 | 26.321 | 13.905 | 56.872 | 1.00 | 20.57 |
| ATOM | 1512 | CG2 | VAL | 226 | 25.283 | 16.016 | 56.005 | 1.00 | 22.66 |
| ATOM | 1513 | C | VAL | 226 | 24.060 | 12.411 | 55.972 | 1.00 | 22.96 |
| ATOM | 1514 | O | VAL | 226 | 24.032 | 12.172 | 57.183 | 1.00 | 23.79 |
| ATOM | 1515 | N | GLY | 227 | 23.924 | 11.470 | 55.054 | 1.00 | 23.08 |
| ATOM | 1516 | CA | GLY | 227 | 23.738 | 10.094 | 55.459 | 1.00 | 25.20 |
| ATOM | 1517 | C | GLY | 227 | 24.623 | 9.207 | 54.621 | 1.00 | 25.79 |
| ATOM | 1518 | O | GLY | 227 | 25.820 | 9.447 | 54.501 | 1.00 | 26.18 |
| ATOM | 1519 | N | THR | 228 | 24.039 | 8.181 | 54.026 | 1.00 | 27.28 |
| ATOM | 1520 | CA | THR | 228 | 24.822 | 7.291 | 53.200 | 1.00 | 29.44 |
| ATOM | 1521 | CB | THR | 228 | 23.900 | 6.356 | 52.413 | 1.00 | 28.91 |
| ATOM | 1522 | OG1 | THR | 228 | 24.691 | 5.441 | 51.650 | 1.00 | 27.54 |
| ATOM | 1523 | CG2 | THR | 228 | 22.983 | 7.159 | 51.496 | 1.00 | 30.69 |
| ATOM | 1524 | C | THR | 228 | 25.705 | 8.139 | 52.267 | 1.00 | 30.87 |
| ATOM | 1525 | O | THR | 228 | 26.878 | 7.834 | 52.072 | 1.00 | 32.00 |
| ATOM | 1526 | N | GLY | 229 | 25.140 | 9.216 | 51.723 | 1.00 | 31.23 |
| ATOM | 1527 | CA | GLY | 229 | 25.888 | 10.111 | 50.855 | 1.00 | 30.25 |
| ATOM | 1528 | C | GLY | 229 | 25.716 | 11.501 | 51.434 | 1.00 | 32.12 |
| ATOM | 1529 | O | GLY | 229 | 25.139 | 11.632 | 52.518 | 1.00 | 33.23 |
| ATOM | 1530 | N | CYS | 230 | 26.208 | 12.535 | 50.749 | 1.00 | 31.95 |
| ATOM | 1531 | CA | CYS | 230 | 26.057 | 13.909 | 51.247 | 1.00 | 31.05 |
| ATOM | 1532 | CB | CYS | 230 | 27.344 | 14.417 | 51.891 | 1.00 | 31.11 |
| ATOM | 1533 | SG | CYS | 230 | 27.145 | 16.090 | 52.562 | 1.00 | 40.64 |
| ATOM | 1534 | C | CYS | 230 | 25.650 | 14.909 | 50.183 | 1.00 | 29.04 |
| ATOM | 1535 | O | CYS | 230 | 26.202 | 14.913 | 49.087 | 1.00 | 30.85 |
| ATOM | 1536 | N | ASN | 231 | 24.701 | 15.775 | 50.513 | 1.00 | 26.12 |
| ATOM | 1537 | CA | ASN | 231 | 24.267 | 16.773 | 49.554 | 1.00 | 26.17 |
| ATOM | 1538 | CB | ASN | 231 | 23.380 | 16.130 | 48.505 | 1.00 | 24.13 |
| ATOM | 1539 | CG | ASN | 231 | 23.146 | 17.030 | 47.341 | 1.00 | 24.98 |
| ATOM | 1540 | OD1 | ASN | 231 | 22.505 | 18.064 | 47.463 | 1.00 | 24.73 |
| ATOM | 1541 | ND2 | ASN | 231 | 23.684 | 16.656 | 46.196 | 1.00 | 29.51 |
| ATOM | 1542 | C | ASN | 231 | 23.529 | 17.927 | 50.213 | 1.00 | 27.77 |
| ATOM | 1543 | O | ASN | 231 | 22.929 | 17.757 | 51.275 | 1.00 | 28.70 |
| ATOM | 1544 | N | ALA | 232 | 23.569 | 19.103 | 49.587 | 1.00 | 27.44 |
| ATOM | 1545 | CA | ALA | 232 | 22.890 | 20.258 | 50.158 | 1.00 | 26.70 |
| ATOM | 1546 | CB | ALA | 232 | 23.806 | 20.963 | 51.113 | 1.00 | 26.89 |
| ATOM | 1547 | C | ALA | 232 | 22.366 | 21.245 | 49.144 | 1.00 | 26.61 |
| ATOM | 1548 | O | ALA | 232 | 22.693 | 21.184 | 47.963 | 1.00 | 26.44 |
| ATOM | 1549 | N | CYS | 233 | 21.537 | 22.161 | 49.617 | 1.00 | 27.04 |
| ATOM | 1550 | CA | CYS | 233 | 20.976 | 23.172 | 48.743 | 1.00 | 31.21 |
| ATOM | 1551 | CB | CYS | 233 | 19.676 | 22.666 | 48.127 | 1.00 | 31.60 |
| ATOM | 1552 | SG | CYS | 233 | 18.376 | 22.446 | 49.348 | 1.00 | 35.31 |
| ATOM | 1553 | C | CYS | 233 | 20.708 | 24.408 | 49.589 | 1.00 | 31.98 |
| ATOM | 1554 | O | CYS | 233 | 20.596 | 24.303 | 50.809 | 1.00 | 32.62 |
| ATOM | 1555 | N | TYR | 234 | 20.621 | 25.572 | 48.949 | 1.00 | 30.70 |
| ATOM | 1556 | CA | TYR | 234 | 20.366 | 26.822 | 49.660 | 1.00 | 30.60 |
| ATOM | 1557 | CB | TYR | 234 | 21.684 | 27.524 | 50.026 | 1.00 | 29.53 |
| ATOM | 1558 | CG | TYR | 234 | 22.464 | 28.011 | 48.829 | 1.00 | 27.41 |
| ATOM | 1559 | CD1 | TYR | 234 | 22.363 | 29.327 | 48.393 | 1.00 | 25.56 |
| ATOM | 1560 | CE1 | TYR | 234 | 22.981 | 29.739 | 47.217 | 1.00 | 25.47 |
| ATOM | 1561 | CD2 | TYR | 234 | 23.218 | 27.121 | 48.061 | 1.00 | 28.10 |
| ATOM | 1562 | CE2 | TYR | 234 | 23.838 | 27.524 | 46.882 | 1.00 | 26.39 |
| ATOM | 1563 | CZ | TYR | 234 | 23.707 | 28.830 | 46.462 | 1.00 | 25.77 |
| ATOM | 1564 | OH | TYR | 234 | 24.240 | 29.201 | 45.253 | 1.00 | 27.36 |
| ATOM | 1565 | C | TYR | 234 | 19.531 | 27.742 | 48.797 | 1.00 | 32.10 |
| ATOM | 1566 | O | TYR | 234 | 19.211 | 27.411 | 47.657 | 1.00 | 32.79 |
| ATOM | 1567 | N | MET | 235 | 19.184 | 28.897 | 49.357 | 1.00 | 34.08 |
| ATOM | 1568 | CA | MET | 235 | 18.380 | 29.908 | 48.679 | 1.00 | 34.57 |
| ATOM | 1569 | CB | MET | 235 | 17.492 | 30.617 | 49.697 | 1.00 | 34.74 |
| ATOM | 1570 | CG | MET | 235 | 16.489 | 29.699 | 50.305 | 1.00 | 34.74 |
| ATOM | 1571 | SD | MET | 235 | 15.575 | 28.985 | 48.959 | 1.00 | 35.81 |
| ATOM | 1572 | CE | MET | 235 | 14.171 | 30.092 | 48.917 | 1.00 | 34.50 |
| ATOM | 1573 | C | MET | 235 | 19.270 | 30.933 | 48.009 | 1.00 | 35.41 |
| ATOM | 1574 | O | MET | 235 | 19.631 | 31.930 | 48.625 | 1.00 | 37.55 |
| ATOM | 1575 | N | GLU | 236 | 19.626 | 30.702 | 46.753 | 1.00 | 35.58 |
| ATOM | 1576 | CA | GLU | 236 | 20.487 | 31.643 | 46.049 | 1.00 | 36.59 |
| ATOM | 1577 | CB | GLU | 236 | 21.168 | 30.949 | 44.869 | 1.00 | 38.16 |
| ATOM | 1578 | CG | GLU | 236 | 22.051 | 31.861 | 44.051 | 1.00 | 39.44 |
| ATOM | 1579 | CD | GLU | 236 | 23.107 | 32.542 | 44.890 | 1.00 | 41.44 |
| ATOM | 1580 | OE1 | GLU | 236 | 24.116 | 31.891 | 45.240 | 1.00 | 40.65 |
| ATOM | 1581 | OE2 | GLU | 236 | 22.918 | 33.735 | 45.208 | 1.00 | 42.03 |
| ATOM | 1582 | C | GLU | 236 | 19.679 | 32.838 | 45.564 | 1.00 | 37.02 |
| ATOM | 1583 | O | GLU | 236 | 18.452 | 32.810 | 45.580 | 1.00 | 38.00 |
| ATOM | 1584 | N | GLU | 237 | 20.354 | 33.898 | 45.149 | 1.00 | 38.75 |
| ATOM | 1585 | CA | GLU | 237 | 19.634 | 35.062 | 44.668 | 1.00 | 41.18 |
| ATOM | 1586 | CB | GLU | 237 | 20.482 | 36.317 | 44.830 | 1.00 | 39.63 |
| ATOM | 1587 | CG | GLU | 237 | 20.912 | 36.579 | 46.258 | 1.00 | 36.10 |
| ATOM | 1588 | CD | GLU | 237 | 19.764 | 37.022 | 47.131 | 1.00 | 35.20 |
| ATOM | 1589 | OE1 | GLU | 237 | 19.056 | 37.971 | 46.726 | 1.00 | 34.49 |
| ATOM | 1590 | OE2 | GLU | 237 | 19.574 | 36.434 | 48.221 | 1.00 | 33.72 |
| ATOM | 1591 | C | GLU | 237 | 19.307 | 34.836 | 43.206 | 1.00 | 43.71 |
| ATOM | 1592 | O | GLU | 237 | 20.143 | 34.351 | 42.437 | 1.00 | 43.65 |
| ATOM | 1593 | N | MET | 238 | 18.078 | 35.172 | 42.832 | 1.00 | 45.47 |
| ATOM | 1594 | CA | MET | 238 | 17.625 | 35.013 | 41.457 | 1.00 | 47.13 |
| ATOM | 1595 | CB | MET | 238 | 16.275 | 35.705 | 41.275 | 1.00 | 47.10 |
| ATOM | 1596 | CG | MET | 238 | 15.094 | 34.875 | 41.721 | 1.00 | 46.82 |
| ATOM | 1597 | SD | MET | 238 | 14.773 | 33.548 | 40.554 | 1.00 | 45.37 |
| ATOM | 1598 | CE | MET | 238 | 13.564 | 34.332 | 39.412 | 1.00 | 46.47 |
| ATOM | 1599 | C | MET | 238 | 18.629 | 35.589 | 40.466 | 1.00 | 48.34 |
| ATOM | 1600 | O | MET | 238 | 18.814 | 35.061 | 39.371 | 1.00 | 49.97 |
| ATOM | 1601 | N | GLN | 239 | 19.280 | 36.672 | 40.868 | 1.00 | 48.44 |
| ATOM | 1602 | CA | GLN | 239 | 20.252 | 37.344 | 40.026 | 1.00 | 49.76 |
| ATOM | 1603 | CB | GLN | 239 | 20.398 | 38.794 | 40.491 | 1.00 | 54.00 |
| ATOM | 1604 | CG | GLN | 239 | 20.375 | 38.963 | 42.007 | 1.00 | 58.66 |
| ATOM | 1605 | CD | GLN | 239 | 20.056 | 40.394 | 42.447 | 1.00 | 63.23 |
| ATOM | 1606 | OE1 | GLN | 239 | 19.660 | 40.624 | 43.593 | 1.00 | 65.75 |
| ATOM | 1607 | NE2 | GLN | 239 | 20.233 | 41.359 | 41.540 | 1.00 | 63.23 |
| ATOM | 1608 | C | GLN | 239 | 21.612 | 36.665 | 40.011 | 1.00 | 48.87 |
| ATOM | 1609 | O | GLN | 239 | 22.611 | 37.295 | 39.687 | 1.00 | 49.50 |
| ATOM | 1610 | N | ASN | 240 | 21.656 | 35.384 | 40.354 | 1.00 | 47.67 |
| ATOM | 1611 | CA | ASN | 240 | 22.926 | 34.660 | 40.379 | 1.00 | 47.01 |
| ATOM | 1612 | CB | ASN | 240 | 23.301 | 34.278 | 41.809 | 1.00 | 47.66 |
| ATOM | 1613 | CG | ASN | 240 | 24.101 | 35.347 | 42.518 | 1.00 | 45.71 |
| ATOM | 1614 | OD1 | ASN | 240 | 23.553 | 36.328 | 43.021 | 1.00 | 43.88 |
| ATOM | 1615 | ND2 | ASN | 240 | 25.414 | 35.159 | 42.561 | 1.00 | 46.64 |
| ATOM | 1616 | C | ASN | 240 | 22.861 | 33.393 | 39.550 | 1.00 | 46.58 |
| ATOM | 1617 | O | ASN | 240 | 23.888 | 32.840 | 39.137 | 1.00 | 46.44 |
| ATOM | 1618 | N | VAL | 241 | 21.643 | 32.919 | 39.340 | 1.00 | 44.69 |
| ATOM | 1619 | CA | VAL | 241 | 21.426 | 31.717 | 38.564 | 1.00 | 43.22 |
| ATOM | 1620 | CB | VAL | 241 | 20.103 | 31.056 | 38.948 | 1.00 | 43.93 |
| ATOM | 1621 | CG1 | VAL | 241 | 20.071 | 29.643 | 38.412 | 1.00 | 44.87 |
| ATOM | 1622 | CG2 | VAL | 241 | 19.922 | 31.091 | 40.456 | 1.00 | 40.98 |
| ATOM | 1623 | C | VAL | 241 | 21.358 | 32.182 | 37.126 | 1.00 | 41.83 |
| ATOM | 1624 | O | VAL | 241 | 20.351 | 32.739 | 36.685 | 1.00 | 42.56 |
| ATOM | 1625 | N | GLU | 242 | 22.433 | 31.974 | 36.386 | 1.00 | 39.79 |
| ATOM | 1626 | CA | GLU | 242 | 22.426 | 32.440 | 35.017 | 1.00 | 38.35 |
| ATOM | 1627 | CB | GLU | 242 | 23.841 | 32.438 | 34.435 | 1.00 | 41.38 |
| ATOM | 1628 | CG | GLU | 242 | 24.874 | 33.080 | 35.345 | 1.00 | 43.21 |
| ATOM | 1629 | CD | GLU | 242 | 26.062 | 33.639 | 34.588 | 1.00 | 46.65 |
| ATOM | 1630 | OE1 | GLU | 242 | 26.489 | 33.026 | 33.581 | 1.00 | 46.29 |
| ATOM | 1631 | OE2 | GLU | 242 | 26.581 | 34.694 | 35.014 | 1.00 | 49.23 |
| ATOM | 1632 | C | GLU | 242 | 21.495 | 31.626 | 34.144 | 1.00 | 34.71 |
| ATOM | 1633 | O | GLU | 242 | 21.135 | 32.057 | 33.054 | 1.00 | 33.08 |
| ATOM | 1634 | N | LEU | 243 | 21.085 | 30.456 | 34.612 | 1.00 | 31.90 |
| ATOM | 1635 | CA | LEU | 243 | 20.194 | 29.652 | 33.794 | 1.00 | 30.72 |
| ATOM | 1636 | CB | LEU | 243 | 20.125 | 28.214 | 34.285 | 1.00 | 29.40 |
| ATOM | 1637 | CG | LEU | 243 | 21.244 | 27.279 | 33.833 | 1.00 | 28.38 |
| ATOM | 1638 | CD1 | LEU | 243 | 21.264 | 27.192 | 32.321 | 1.00 | 23.84 |
| ATOM | 1639 | CD2 | LEU | 243 | 22.570 | 27.786 | 34.381 | 1.00 | 31.28 |
| ATOM | 1640 | C | LEU | 243 | 18.799 | 30.222 | 33.763 | 1.00 | 31.18 |
| ATOM | 1641 | O | LEU | 243 | 18.143 | 30.153 | 32.729 | 1.00 | 32.86 |
| ATOM | 1642 | N | VAL | 244 | 18.350 | 30.779 | 34.887 | 1.00 | 30.11 |
| ATOM | 1643 | CA | VAL | 244 | 17.011 | 31.361 | 34.979 | 1.00 | 30.23 |
| ATOM | 1644 | CB | VAL | 244 | 16.549 | 31.527 | 36.432 | 1.00 | 31.77 |
| ATOM | 1645 | CG1 | VAL | 244 | 15.085 | 31.981 | 36.444 | 1.00 | 31.84 |
| ATOM | 1646 | CG2 | VAL | 244 | 16.748 | 30.234 | 37.213 | 1.00 | 31.59 |
| ATOM | 1647 | C | VAL | 244 | 16.955 | 32.746 | 34.361 | 1.00 | 30.94 |
| ATOM | 1648 | O | VAL | 244 | 17.919 | 33.499 | 34.458 | 1.00 | 31.77 |
| ATOM | 1649 | N | GLU | 245 | 15.819 | 33.083 | 33.753 | 1.00 | 32.44 |
| ATOM | 1650 | CA | GLU | 245 | 15.625 | 34.389 | 33.125 | 1.00 | 36.05 |
| ATOM | 1651 | CB | GLU | 245 | 14.384 | 34.384 | 32.237 | 1.00 | 35.98 |
| ATOM | 1652 | CG | GLU | 245 | 14.542 | 35.203 | 30.981 | 1.00 | 38.72 |
| ATOM | 1653 | CD | GLU | 245 | 15.357 | 34.449 | 29.959 | 1.00 | 41.52 |
| ATOM | 1654 | OE1 | GLU | 245 | 15.957 | 33.428 | 30.356 | 1.00 | 40.02 |
| ATOM | 1655 | OE2 | GLU | 245 | 15.402 | 34.859 | 28.779 | 1.00 | 43.26 |
| ATOM | 1656 | C | GLU | 245 | 15.453 | 35.511 | 34.149 | 1.00 | 39.49 |
| ATOM | 1657 | O | GLU | 245 | 15.995 | 36.603 | 33.978 | 1.00 | 39.69 |
| ATOM | 1658 | N | GLY | 246 | 14.676 | 35.239 | 35.197 | 1.00 | 42.62 |
| ATOM | 1659 | CA | GLY | 246 | 14.417 | 36.228 | 36.233 | 1.00 | 44.14 |
| ATOM | 1660 | C | GLY | 246 | 15.642 | 36.762 | 36.953 | 1.00 | 44.54 |
| ATOM | 1661 | O | GLY | 246 | 16.720 | 36.163 | 36.906 | 1.00 | 43.59 |
| ATOM | 1662 | N | ASP | 247 | 15.476 | 37.896 | 37.627 | 1.00 | 44.51 |
| ATOM | 1663 | CA | ASP | 247 | 16.582 | 38.500 | 38.345 | 1.00 | 45.26 |
| ATOM | 1664 | CB | ASP | 247 | 17.179 | 39.654 | 37.540 | 1.00 | 48.06 |
| ATOM | 1665 | CG | ASP | 247 | 18.102 | 39.173 | 36.436 | 1.00 | 52.60 |
| ATOM | 1666 | OD1 | ASP | 247 | 19.016 | 38.376 | 36.744 | 1.00 | 54.76 |
| ATOM | 1667 | OD2 | ASP | 247 | 17.923 | 39.584 | 35.265 | 1.00 | 54.15 |
| ATOM | 1668 | C | ASP | 247 | 16.213 | 38.993 | 39.720 | 1.00 | 44.83 |
| ATOM | 1669 | O | ASP | 247 | 17.087 | 39.306 | 40.518 | 1.00 | 45.80 |
| ATOM | 1670 | N | GLU | 248 | 14.930 | 39.064 | 40.022 | 1.00 | 44.56 |
| ATOM | 1671 | CA | GLU | 248 | 14.561 | 39.546 | 41.336 | 1.00 | 45.70 |
| ATOM | 1672 | CB | GLU | 248 | 13.610 | 40.727 | 41.206 | 1.00 | 50.66 |
| ATOM | 1673 | CG | GLU | 248 | 12.441 | 40.458 | 40.298 | 1.00 | 60.84 |
| ATOM | 1674 | CD | GLU | 248 | 11.394 | 41.556 | 40.355 | 1.00 | 67.29 |
| ATOM | 1675 | OE1 | GLU | 248 | 10.742 | 41.702 | 41.414 | 1.00 | 69.90 |
| ATOM | 1676 | OE2 | GLU | 248 | 11.223 | 42.273 | 39.340 | 1.00 | 71.41 |
| ATOM | 1677 | C | GLU | 248 | 13.952 | 38.482 | 42.224 | 1.00 | 43.15 |
| ATOM | 1678 | O | GLU | 248 | 12.986 | 37.827 | 41.855 | 1.00 | 42.29 |
| ATOM | 1679 | N | GLY | 249 | 14.530 | 38.315 | 43.404 | 1.00 | 42.35 |
| ATOM | 1680 | CA | GLY | 249 | 14.023 | 37.327 | 44.330 | 1.00 | 42.91 |
| ATOM | 1681 | C | GLY | 249 | 15.044 | 36.247 | 44.625 | 1.00 | 43.93 |
| ATOM | 1682 | O | GLY | 249 | 16.177 | 36.294 | 44.145 | 1.00 | 43.62 |
| ATOM | 1683 | N | ARG | 250 | 14.644 | 35.267 | 45.427 | 1.00 | 43.38 |
| ATOM | 1684 | CA | ARG | 250 | 15.526 | 34.160 | 45.781 | 1.00 | 41.04 |
| ATOM | 1685 | CB | ARG | 250 | 15.819 | 34.207 | 47.293 | 1.00 | 42.27 |
| ATOM | 1686 | CG | ARG | 250 | 14.745 | 34.934 | 48.114 | 1.00 | 46.82 |
| ATOM | 1687 | CD | ARG | 250 | 15.139 | 35.142 | 49.584 | 1.00 | 51.21 |
| ATOM | 1688 | NE | ARG | 250 | 16.425 | 35.828 | 49.730 | 1.00 | 55.52 |
| ATOM | 1689 | CZ | ARG | 250 | 16.864 | 36.394 | 50.855 | 1.00 | 55.63 |
| ATOM | 1690 | NH1 | ARG | 250 | 16.121 | 36.375 | 51.956 | 1.00 | 55.05 |
| ATOM | 1691 | NH2 | ARG | 250 | 18.063 | 36.962 | 50.885 | 1.00 | 54.32 |
| ATOM | 1692 | C | ARG | 250 | 14.905 | 32.812 | 45.359 | 1.00 | 38.25 |
| ATOM | 1693 | O | ARG | 250 | 13.681 | 32.640 | 45.394 | 1.00 | 37.44 |
| ATOM | 1694 | N | MET | 251 | 15.760 | 31.880 | 44.932 | 1.00 | 33.58 |
| ATOM | 1695 | CA | MET | 251 | 15.352 | 30.543 | 44.492 | 1.00 | 29.34 |
| ATOM | 1696 | CB | MET | 251 | 15.326 | 30.471 | 42.966 | 1.00 | 24.54 |
| ATOM | 1697 | CG | MET | 251 | 15.180 | 29.069 | 42.379 | 1.00 | 17.89 |
| ATOM | 1698 | SD | MET | 251 | 14.994 | 29.090 | 40.552 | 1.00 | 18.23 |
| ATOM | 1699 | CE | MET | 251 | 16.329 | 28.087 | 40.075 | 1.00 | 12.48 |
| ATOM | 1700 | C | MET | 251 | 16.316 | 29.481 | 45.004 | 1.00 | 30.48 |
| ATOM | 1701 | O | MET | 251 | 17.529 | 29.640 | 44.895 | 1.00 | 31.49 |
| ATOM | 1702 | N | CYS | 252 | 15.775 | 28.392 | 45.546 | 1.00 | 29.56 |
| ATOM | 1703 | CA | CYS | 252 | 16.599 | 27.298 | 46.059 | 1.00 | 26.54 |
| ATOM | 1704 | CB | CYS | 252 | 15.710 | 26.185 | 46.612 | 1.00 | 27.29 |
| ATOM | 1705 | SG | CYS | 252 | 16.613 | 24.659 | 46.927 | 1.00 | 29.14 |
| ATOM | 1706 | C | CYS | 252 | 17.492 | 26.704 | 44.975 | 1.00 | 23.38 |
| ATOM | 1707 | O | CYS | 252 | 17.104 | 26.639 | 43.816 | 1.00 | 22.79 |
| ATOM | 1708 | N | VAL | 253 | 18.688 | 26.268 | 45.349 | 1.00 | 20.80 |
| ATOM | 1709 | CA | VAL | 253 | 19.584 | 25.660 | 44.377 | 1.00 | 20.25 |
| ATOM | 1710 | CB | VAL | 253 | 20.740 | 26.583 | 43.969 | 1.00 | 19.02 |
| ATOM | 1711 | CG1 | VAL | 253 | 21.623 | 25.881 | 42.936 | 1.00 | 15.42 |
| ATOM | 1712 | CG2 | VAL | 253 | 20.198 | 27.866 | 43.411 | 1.00 | 19.77 |
| ATOM | 1713 | C | VAL | 253 | 20.191 | 24.374 | 44.900 | 1.00 | 22.35 |
| ATOM | 1714 | O | VAL | 253 | 20.705 | 24.305 | 46.023 | 1.00 | 22.21 |
| ATOM | 1715 | N | ASN | 254 | 20.127 | 23.352 | 44.060 | 1.00 | 24.23 |
| ATOM | 1716 | CA | ASN | 254 | 20.661 | 22.045 | 44.390 | 1.00 | 22.10 |
| ATOM | 1717 | CB | ASN | 254 | 19.860 | 20.975 | 43.647 | 1.00 | 21.49 |
| ATOM | 1718 | CG | ASN | 254 | 20.479 | 19.604 | 43.747 | 1.00 | 22.93 |
| ATOM | 1719 | OD1 | ASN | 254 | 21.074 | 19.232 | 44.764 | 1.00 | 20.03 |
| ATOM | 1720 | ND2 | ASN | 254 | 20.325 | 18.827 | 42.687 | 1.00 | 26.40 |
| ATOM | 1721 | C | ASN | 254 | 22.124 | 22.046 | 43.975 | 1.00 | 19.26 |
| ATOM | 1722 | O | ASN | 254 | 22.454 | 22.155 | 42.795 | 1.00 | 15.88 |
| ATOM | 1723 | N | THR | 255 | 23.001 | 21.949 | 44.961 | 1.00 | 15.23 |
| ATOM | 1724 | CA | THR | 255 | 24.428 | 21.962 | 44.691 | 1.00 | 15.03 |
| ATOM | 1725 | CB | THR | 255 | 25.193 | 22.217 | 45.944 | 1.00 | 13.56 |
| ATOM | 1726 | OG1 | THR | 255 | 25.035 | 21.087 | 46.808 | 1.00 | 14.56 |
| ATOM | 1727 | CG2 | THR | 255 | 24.670 | 23.458 | 46.617 | 1.00 | 14.18 |
| ATOM | 1728 | C | THR | 255 | 24.957 | 20.665 | 44.127 | 1.00 | 15.21 |
| ATOM | 1729 | O | THR | 255 | 25.675 | 20.647 | 43.126 | 1.00 | 12.07 |
| ATOM | 1730 | N | GLU | 256 | 24.594 | 19.570 | 44.777 | 1.00 | 18.83 |
| ATOM | 1731 | CA | GLU | 256 | 25.076 | 18.268 | 44.355 | 1.00 | 22.28 |
| ATOM | 1732 | CB | GLU | 256 | 24.795 | 18.025 | 42.876 | 1.00 | 25.93 |
| ATOM | 1733 | CG | GLU | 256 | 23.377 | 18.345 | 42.454 | 1.00 | 31.90 |
| ATOM | 1734 | CD | GLU | 256 | 22.500 | 17.121 | 42.336 | 1.00 | 34.74 |
| ATOM | 1735 | OE1 | GLU | 256 | 22.191 | 16.510 | 43.386 | 1.00 | 36.97 |
| ATOM | 1736 | OE2 | GLU | 256 | 22.122 | 16.777 | 41.188 | 1.00 | 35.26 |
| ATOM | 1737 | C | GLU | 256 | 26.562 | 18.402 | 44.559 | 1.00 | 21.32 |
| ATOM | 1738 | O | GLU | 256 | 27.359 | 18.032 | 43.701 | 1.00 | 23.09 |
| ATOM | 1739 | N | TRP | 257 | 26.931 | 18.966 | 45.699 | 1.00 | 17.36 |
| ATOM | 1740 | CA | TRP | 257 | 28.327 | 19.141 | 45.985 | 1.00 | 14.83 |
| ATOM | 1741 | CB | TRP | 257 | 28.514 | 20.074 | 47.176 | 1.00 | 11.59 |
| ATOM | 1742 | CG | TRP | 257 | 28.038 | 19.561 | 48.478 | 1.00 | 8.69 |
| ATOM | 1743 | CD2 | TRP | 257 | 27.830 | 20.332 | 49.676 | 1.00 | 9.05 |
| ATOM | 1744 | CE2 | TRP | 257 | 27.562 | 19.410 | 50.715 | 1.00 | 7.00 |
| ATOM | 1745 | CE3 | TRP | 257 | 27.845 | 21.703 | 49.964 | 1.00 | 7.18 |
| ATOM | 1746 | CD1 | TRP | 257 | 27.881 | 18.265 | 48.827 | 1.00 | 7.58 |
| ATOM | 1747 | NE1 | TRP | 257 | 27.602 | 18.163 | 50.172 | 1.00 | 7.99 |
| ATOM | 1748 | CZ2 | TRP | 257 | 27.325 | 19.818 | 52.038 | 1.00 | 4.73 |
| ATOM | 1749 | CZ3 | TRP | 257 | 27.605 | 22.108 | 51.280 | 1.00 | 7.12 |
| ATOM | 1750 | CH2 | TRP | 257 | 27.346 | 21.164 | 52.300 | 1.00 | 5.47 |
| ATOM | 1751 | C | TRP | 257 | 29.033 | 17.813 | 46.224 | 1.00 | 17.81 |
| ATOM | 1752 | O | TRP | 257 | 30.221 | 17.776 | 46.523 | 1.00 | 19.44 |
| ATOM | 1753 | N | GLY | 258 | 28.318 | 16.708 | 46.099 | 1.00 | 21.88 |
| ATOM | 1754 | CA | GLY | 258 | 28.991 | 15.444 | 46.303 | 1.00 | 23.25 |
| ATOM | 1755 | C | GLY | 258 | 30.137 | 15.303 | 45.316 | 1.00 | 23.01 |
| ATOM | 1756 | O | GLY | 258 | 31.133 | 14.629 | 45.600 | 1.00 | 21.92 |
| ATOM | 1757 | N | ALA | 259 | 29.997 | 15.943 | 44.156 | 1.00 | 23.11 |
| ATOM | 1758 | CA | ALA | 259 | 31.015 | 15.863 | 43.113 | 1.00 | 27.74 |
| ATOM | 1759 | CB | ALA | 259 | 30.400 | 16.139 | 41.766 | 1.00 | 27.03 |
| ATOM | 1760 | C | ALA | 259 | 32.176 | 16.806 | 43.335 | 1.00 | 30.23 |
| ATOM | 1761 | O | ALA | 259 | 33.178 | 16.748 | 42.622 | 1.00 | 32.12 |
| ATOM | 1762 | N | PHE | 260 | 32.041 | 17.680 | 44.320 | 1.00 | 32.43 |
| ATOM | 1763 | CA | PHE | 260 | 33.093 | 18.627 | 44.611 | 1.00 | 36.43 |
| ATOM | 1764 | CB | PHE | 260 | 32.804 | 19.343 | 45.924 | 1.00 | 39.42 |
| ATOM | 1765 | CG | PHE | 260 | 33.932 | 20.206 | 46.411 | 1.00 | 43.92 |
| ATOM | 1766 | CD1 | PHE | 260 | 34.660 | 21.003 | 45.534 | 1.00 | 46.49 |
| ATOM | 1767 | CD2 | PHE | 260 | 34.232 | 20.263 | 47.765 | 1.00 | 45.64 |
| ATOM | 1768 | CE1 | PHE | 260 | 35.672 | 21.835 | 46.002 | 1.00 | 47.73 |
| ATOM | 1769 | CE2 | PHE | 260 | 35.242 | 21.093 | 48.242 | 1.00 | 46.62 |
| ATOM | 1770 | CZ | PHE | 260 | 35.958 | 21.882 | 47.360 | 1.00 | 47.27 |
| ATOM | 1771 | C | PHE | 260 | 34.412 | 17.897 | 44.695 | 1.00 | 39.39 |
| ATOM | 1772 | O | PHE | 260 | 34.495 | 16.800 | 45.243 | 1.00 | 40.20 |
| ATOM | 1773 | N | GLY | 261 | 35.441 | 18.511 | 44.127 | 1.00 | 41.71 |
| ATOM | 1774 | CA | GLY | 261 | 36.753 | 17.911 | 44.152 | 1.00 | 43.62 |
| ATOM | 1775 | C | GLY | 261 | 36.967 | 16.857 | 43.090 | 1.00 | 44.99 |
| ATOM | 1776 | O | GLY | 261 | 38.049 | 16.282 | 43.015 | 1.00 | 47.22 |
| ATOM | 1777 | N | ASP | 262 | 35.961 | 16.578 | 42.270 | 1.00 | 46.06 |
| ATOM | 1778 | CA | ASP | 262 | 36.143 | 15.574 | 41.229 | 1.00 | 47.68 |
| ATOM | 1779 | CB | ASP | 262 | 34.800 | 15.197 | 40.602 | 1.00 | 50.82 |
| ATOM | 1780 | CG | ASP | 262 | 34.024 | 14.187 | 41.445 | 1.00 | 53.64 |
| ATOM | 1781 | OD1 | ASP | 262 | 32.815 | 13.996 | 41.191 | 1.00 | 54.63 |
| ATOM | 1782 | OD2 | ASP | 262 | 34.624 | 13.578 | 42.356 | 1.00 | 54.71 |
| ATOM | 1783 | C | ASP | 262 | 37.089 | 16.129 | 40.177 | 1.00 | 47.19 |
| ATOM | 1784 | O | ASP | 262 | 37.539 | 15.400 | 39.292 | 1.00 | 47.09 |
| ATOM | 1785 | N | SER | 263 | 37.380 | 17.427 | 40.298 | 1.00 | 46.38 |
| ATOM | 1786 | CA | SER | 263 | 38.289 | 18.147 | 39.401 | 1.00 | 44.53 |
| ATOM | 1787 | CB | SER | 263 | 37.651 | 19.445 | 38.903 | 1.00 | 43.57 |
| ATOM | 1788 | OG | SER | 263 | 36.341 | 19.246 | 38.415 | 1.00 | 43.79 |
| ATOM | 1789 | C | SER | 263 | 39.552 | 18.513 | 40.174 | 1.00 | 43.93 |
| ATOM | 1790 | O | SER | 263 | 40.061 | 19.632 | 40.059 | 1.00 | 44.40 |
| ATOM | 1791 | N | GLY | 264 | 40.039 | 17.577 | 40.979 | 1.00 | 43.71 |
| ATOM | 1792 | CA | GLY | 264 | 41.235 | 17.825 | 41.762 | 1.00 | 42.64 |
| ATOM | 1793 | C | GLY | 264 | 41.133 | 18.889 | 42.845 | 1.00 | 40.75 |
| ATOM | 1794 | O | GLY | 264 | 42.052 | 19.012 | 43.648 | 1.00 | 42.90 |
| ATOM | 1795 | N | GLU | 265 | 40.040 | 19.647 | 42.887 | 1.00 | 38.43 |
| ATOM | 1796 | CA | GLU | 265 | 39.881 | 20.700 | 43.893 | 1.00 | 37.42 |
| ATOM | 1797 | CB | GLU | 265 | 38.437 | 21.227 | 43.907 | 1.00 | 39.11 |
| ATOM | 1798 | CG | GLU | 265 | 37.986 | 21.928 | 42.632 | 1.00 | 40.76 |
| ATOM | 1799 | CD | GLU | 265 | 37.198 | 21.023 | 41.701 | 1.00 | 43.56 |
| ATOM | 1800 | OE1 | GLU | 265 | 36.904 | 21.461 | 40.565 | 1.00 | 45.26 |
| ATOM | 1801 | OE2 | GLU | 265 | 36.863 | 19.883 | 42.099 | 1.00 | 42.42 |
| ATOM | 1802 | C | GLU | 265 | 40.266 | 20.299 | 45.321 | 1.00 | 36.38 |
| ATOM | 1803 | O | GLU | 265 | 40.410 | 21.160 | 46.185 | 1.00 | 33.59 |
| ATOM | 1804 | N | LEU | 266 | 40.425 | 19.004 | 45.573 | 1.00 | 37.71 |
| ATOM | 1805 | CA | LEU | 266 | 40.783 | 18.534 | 46.912 | 1.00 | 40.56 |
| ATOM | 1806 | CB | LEU | 266 | 39.597 | 17.831 | 47.567 | 1.00 | 40.03 |
| ATOM | 1807 | CG | LEU | 266 | 38.371 | 18.631 | 48.001 | 1.00 | 40.79 |
| ATOM | 1808 | CD1 | LEU | 266 | 37.234 | 17.673 | 48.259 | 1.00 | 40.27 |
| ATOM | 1809 | CD2 | LEU | 266 | 38.677 | 19.432 | 49.253 | 1.00 | 41.81 |
| ATOM | 1810 | C | LEU | 266 | 41.949 | 17.563 | 46.880 | 1.00 | 43.51 |
| ATOM | 1811 | O | LEU | 266 | 42.363 | 17.045 | 47.919 | 1.00 | 43.63 |
| ATOM | 1812 | N | ASP | 267 | 42.475 | 17.324 | 45.682 | 1.00 | 47.00 |
| ATOM | 1813 | CA | ASP | 267 | 43.584 | 16.393 | 45.480 | 1.00 | 48.18 |
| ATOM | 1814 | CB | ASP | 267 | 44.222 | 16.622 | 44.097 | 1.00 | 50.89 |
| ATOM | 1815 | CG | ASP | 267 | 44.982 | 15.391 | 43.584 | 1.00 | 54.98 |
| ATOM | 1816 | OD1 | ASP | 267 | 45.239 | 15.317 | 42.360 | 1.00 | 56.65 |
| ATOM | 1817 | OD2 | ASP | 267 | 45.328 | 14.499 | 44.398 | 1.00 | 55.43 |
| ATOM | 1818 | C | ASP | 267 | 44.659 | 16.440 | 46.571 | 1.00 | 46.46 |
| ATOM | 1819 | O | ASP | 267 | 45.205 | 15.397 | 46.960 | 1.00 | 45.37 |
| ATOM | 1820 | N | GLU | 268 | 44.957 | 17.630 | 47.084 | 1.00 | 44.63 |
| ATOM | 1821 | CA | GLU | 268 | 45.990 | 17.721 | 48.109 | 1.00 | 44.67 |
| ATOM | 1822 | CB | GLU | 268 | 46.805 | 19.024 | 47.956 | 1.00 | 44.68 |
| ATOM | 1823 | CG | GLU | 268 | 46.508 | 20.163 | 48.934 | 1.00 | 43.60 |
| ATOM | 1824 | CD | GLU | 268 | 45.234 | 20.915 | 48.613 | 1.00 | 43.53 |
| ATOM | 1825 | OE1 | GLU | 268 | 45.020 | 21.258 | 47.423 | 1.00 | 42.43 |
| ATOM | 1826 | OE2 | GLU | 268 | 44.461 | 21.174 | 49.561 | 1.00 | 40.84 |
| ATOM | 1827 | C | GLU | 268 | 45.457 | 17.569 | 49.528 | 1.00 | 43.45 |
| ATOM | 1828 | O | GLU | 268 | 46.102 | 17.961 | 50.499 | 1.00 | 46.29 |
| ATOM | 1829 | N | PHE | 269 | 44.286 | 16.971 | 49.656 | 1.00 | 38.78 |
| ATOM | 1830 | CA | PHE | 269 | 43.729 | 16.785 | 50.974 | 1.00 | 33.75 |
| ATOM | 1831 | CB | PHE | 269 | 42.480 | 17.614 | 51.135 | 1.00 | 33.69 |
| ATOM | 1832 | CG | PHE | 269 | 42.733 | 18.990 | 51.639 | 1.00 | 34.75 |
| ATOM | 1833 | CD1 | PHE | 269 | 43.435 | 19.193 | 52.822 | 1.00 | 36.51 |
| ATOM | 1834 | CD2 | PHE | 269 | 42.161 | 20.079 | 51.001 | 1.00 | 34.78 |
| ATOM | 1835 | CE1 | PHE | 269 | 43.548 | 20.469 | 53.365 | 1.00 | 37.39 |
| ATOM | 1836 | CE2 | PHE | 269 | 42.266 | 21.354 | 51.532 | 1.00 | 35.15 |
| ATOM | 1837 | CZ | PHE | 269 | 42.955 | 21.551 | 52.717 | 1.00 | 37.68 |
| ATOM | 1838 | C | PHE | 269 | 43.405 | 15.343 | 51.225 | 1.00 | 32.83 |
| ATOM | 1839 | O | PHE | 269 | 43.206 | 14.952 | 52.365 | 1.00 | 31.85 |
| ATOM | 1840 | N | LEU | 270 | 43.355 | 14.555 | 50.157 | 1.00 | 33.85 |
| ATOM | 1841 | CA | LEU | 270 | 43.046 | 13.130 | 50.259 | 1.00 | 34.53 |
| ATOM | 1842 | CB | LEU | 270 | 42.712 | 12.553 | 48.884 | 1.00 | 35.63 |
| ATOM | 1843 | CG | LEU | 270 | 41.326 | 12.857 | 48.321 | 1.00 | 37.61 |
| ATOM | 1844 | CD1 | LEU | 270 | 41.323 | 14.293 | 47.842 | 1.00 | 35.85 |
| ATOM | 1845 | CD2 | LEU | 270 | 40.966 | 11.878 | 47.177 | 1.00 | 37.50 |
| ATOM | 1846 | C | LEU | 270 | 44.172 | 12.298 | 50.845 | 1.00 | 33.68 |
| ATOM | 1847 | O | LEU | 270 | 45.334 | 12.640 | 50.695 | 1.00 | 35.64 |
| ATOM | 1848 | N | LEU | 271 | 43.829 | 11.200 | 51.507 | 1.00 | 33.66 |
| ATOM | 1849 | CA | LEU | 271 | 44.850 | 10.324 | 52.059 | 1.00 | 34.55 |
| ATOM | 1850 | CB | LEU | 271 | 44.610 | 10.032 | 53.519 | 1.00 | 30.63 |
| ATOM | 1851 | CG | LEU | 271 | 44.870 | 11.238 | 54.383 | 1.00 | 29.49 |
| ATOM | 1852 | CD1 | LEU | 271 | 43.855 | 12.324 | 54.075 | 1.00 | 27.82 |
| ATOM | 1853 | CD2 | LEU | 271 | 44.783 | 10.798 | 55.824 | 1.00 | 31.04 |
| ATOM | 1854 | C | LEU | 271 | 44.884 | 9.010 | 51.324 | 1.00 | 37.04 |
| ATOM | 1855 | O | LEU | 271 | 44.009 | 8.715 | 50.513 | 1.00 | 36.79 |
| ATOM | 1856 | N | GLU | 272 | 45.890 | 8.209 | 51.638 | 1.00 | 40.66 |
| ATOM | 1857 | CA | GLU | 272 | 46.052 | 6.927 | 50.989 | 1.00 | 44.99 |
| ATOM | 1858 | CB | GLU | 272 | 47.256 | 6.182 | 51.590 | 1.00 | 51.18 |
| ATOM | 1859 | CG | GLU | 272 | 47.124 | 5.781 | 53.075 | 1.00 | 58.46 |
| ATOM | 1860 | CD | GLU | 272 | 48.371 | 5.077 | 53.641 | 1.00 | 62.56 |
| ATOM | 1861 | OE1 | GLU | 272 | 49.393 | 5.772 | 53.876 | 1.00 | 64.96 |
| ATOM | 1862 | OE2 | GLU | 272 | 48.325 | 3.835 | 53.849 | 1.00 | 61.73 |
| ATOM | 1863 | C | GLU | 272 | 44.789 | 6.080 | 51.092 | 1.00 | 44.62 |
| ATOM | 1864 | O | GLU | 272 | 44.377 | 5.452 | 50.116 | 1.00 | 44.50 |
| ATOM | 1865 | N | TYR | 273 | 44.163 | 6.079 | 52.266 | 1.00 | 43.42 |
| ATOM | 1866 | CA | TYR | 273 | 42.955 | 5.284 | 52.486 | 1.00 | 40.23 |
| ATOM | 1867 | CB | TYR | 273 | 42.537 | 5.377 | 53.958 | 1.00 | 38.82 |
| ATOM | 1868 | CG | TYR | 273 | 43.709 | 5.401 | 54.923 | 1.00 | 36.38 |
| ATOM | 1869 | CD1 | TYR | 273 | 44.126 | 6.602 | 55.505 | 1.00 | 35.57 |
| ATOM | 1870 | CE1 | TYR | 273 | 45.210 | 6.647 | 56.380 | 1.00 | 34.95 |
| ATOM | 1871 | CD2 | TYR | 273 | 44.413 | 4.231 | 55.243 | 1.00 | 35.34 |
| ATOM | 1872 | CE2 | TYR | 273 | 45.509 | 4.264 | 56.122 | 1.00 | 34.05 |
| ATOM | 1873 | CZ | TYR | 273 | 45.897 | 5.481 | 56.685 | 1.00 | 34.66 |
| ATOM | 1874 | OH | TYR | 273 | 46.966 | 5.556 | 57.550 | 1.00 | 33.77 |
| ATOM | 1875 | C | TYR | 273 | 41.826 | 5.749 | 51.567 | 1.00 | 38.50 |
| ATOM | 1876 | O | TYR | 273 | 41.264 | 4.967 | 50.804 | 1.00 | 35.21 |
| ATOM | 1877 | N | ASP | 274 | 41.507 | 7.030 | 51.638 | 1.00 | 38.17 |
| ATOM | 1878 | CA | ASP | 274 | 40.473 | 7.579 | 50.796 | 1.00 | 40.03 |
| ATOM | 1879 | CB | ASP | 274 | 40.470 | 9.083 | 50.929 | 1.00 | 41.17 |
| ATOM | 1880 | CG | ASP | 274 | 40.252 | 9.512 | 52.341 | 1.00 | 43.77 |
| ATOM | 1881 | OD1 | ASP | 274 | 39.123 | 9.327 | 52.839 | 1.00 | 46.59 |
| ATOM | 1882 | OD2 | ASP | 274 | 41.212 | 10.010 | 52.958 | 1.00 | 44.41 |
| ATOM | 1883 | C | ASP | 274 | 40.740 | 7.200 | 49.359 | 1.00 | 40.92 |
| ATOM | 1884 | O | ASP | 274 | 39.819 | 6.937 | 48.595 | 1.00 | 41.41 |
| ATOM | 1885 | N | ARG | 275 | 42.007 | 7.160 | 48.984 | 1.00 | 42.93 |
| ATOM | 1886 | CA | ARG | 275 | 42.333 | 6.819 | 47.613 | 1.00 | 45.81 |
| ATOM | 1887 | CB | ARG | 275 | 43.831 | 6.993 | 47.365 | 1.00 | 49.53 |
| ATOM | 1888 | CG | ARG | 275 | 44.191 | 7.563 | 45.995 | 1.00 | 53.24 |
| ATOM | 1889 | CD | ARG | 275 | 45.702 | 7.772 | 45.886 | 1.00 | 58.85 |
| ATOM | 1890 | NE | ARG | 275 | 46.213 | 8.663 | 46.933 | 1.00 | 62.67 |
| ATOM | 1891 | CZ | ARG | 275 | 47.088 | 8.308 | 47.876 | 1.00 | 62.82 |
| ATOM | 1892 | NH1 | ARG | 275 | 47.571 | 7.068 | 47.922 | 1.00 | 61.28 |
| ATOM | 1893 | NH2 | ARG | 275 | 47.476 | 9.201 | 48.777 | 1.00 | 61.64 |
| ATOM | 1894 | C | ARG | 275 | 41.901 | 5.390 | 47.316 | 1.00 | 46.01 |
| ATOM | 1895 | O | ARG | 275 | 41.134 | 5.160 | 46.382 | 1.00 | 45.19 |
| ATOM | 1896 | N | LEU | 276 | 42.382 | 4.437 | 48.113 | 1.00 | 47.51 |
| ATOM | 1897 | CA | LEU | 276 | 42.026 | 3.030 | 47.922 | 1.00 | 48.68 |
| ATOM | 1898 | CB | LEU | 276 | 42.460 | 2.197 | 49.134 | 1.00 | 45.63 |
| ATOM | 1899 | CG | LEU | 276 | 43.971 | 1.999 | 49.287 | 1.00 | 43.28 |
| ATOM | 1900 | CD1 | LEU | 276 | 44.418 | 2.379 | 50.686 | 1.00 | 42.53 |
| ATOM | 1901 | CD2 | LEU | 276 | 44.321 | 0.557 | 48.994 | 1.00 | 42.97 |
| ATOM | 1902 | C | LEU | 276 | 40.520 | 2.915 | 47.718 | 1.00 | 51.24 |
| ATOM | 1903 | O | LEU | 276 | 40.050 | 2.133 | 46.891 | 1.00 | 52.38 |
| ATOM | 1904 | N | VAL | 277 | 39.772 | 3.710 | 48.475 | 1.00 | 53.11 |
| ATOM | 1905 | CA | VAL | 277 | 38.321 | 3.722 | 48.372 | 1.00 | 54.05 |
| ATOM | 1906 | CB | VAL | 277 | 37.703 | 4.640 | 49.423 | 1.00 | 52.84 |
| ATOM | 1907 | CG1 | VAL | 277 | 36.210 | 4.682 | 49.249 | 1.00 | 52.71 |
| ATOM | 1908 | CG2 | VAL | 277 | 38.069 | 4.156 | 50.804 | 1.00 | 54.87 |
| ATOM | 1909 | C | VAL | 277 | 37.906 | 4.231 | 46.999 | 1.00 | 55.80 |
| ATOM | 1910 | O | VAL | 277 | 37.381 | 3.474 | 46.185 | 1.00 | 57.15 |
| ATOM | 1911 | N | ASP | 278 | 38.146 | 5.518 | 46.754 | 1.00 | 56.71 |
| ATOM | 1912 | CA | ASP | 278 | 37.804 | 6.146 | 45.481 | 1.00 | 57.65 |
| ATOM | 1913 | CB | ASP | 278 | 38.479 | 7.514 | 45.353 | 1.00 | 59.73 |
| ATOM | 1914 | CG | ASP | 278 | 38.243 | 8.163 | 43.989 | 1.00 | 61.93 |
| ATOM | 1915 | OD1 | ASP | 278 | 38.990 | 9.110 | 43.642 | 1.00 | 61.47 |
| ATOM | 1916 | OD2 | ASP | 278 | 37.308 | 7.733 | 43.273 | 1.00 | 62.11 |
| ATOM | 1917 | C | ASP | 278 | 38.263 | 5.281 | 44.328 | 1.00 | 58.14 |
| ATOM | 1918 | O | ASP | 278 | 37.645 | 5.271 | 43.266 | 1.00 | 58.75 |
| ATOM | 1919 | N | GLU | 279 | 39.358 | 4.563 | 44.538 | 1.00 | 58.33 |
| ATOM | 1920 | CA | GLU | 279 | 39.900 | 3.710 | 43.498 | 1.00 | 59.14 |
| ATOM | 1921 | CB | GLU | 279 | 41.437 | 3.808 | 43.477 | 1.00 | 60.99 |
| ATOM | 1922 | CG | GLU | 279 | 41.978 | 5.219 | 43.178 | 1.00 | 61.92 |
| ATOM | 1923 | CD | GLU | 279 | 43.497 | 5.276 | 43.014 | 1.00 | 60.92 |
| ATOM | 1924 | OE1 | GLU | 279 | 44.219 | 4.874 | 43.953 | 1.00 | 60.85 |
| ATOM | 1925 | OE2 | GLU | 279 | 43.965 | 5.733 | 41.946 | 1.00 | 58.99 |
| ATOM | 1926 | C | GLU | 279 | 39.467 | 2.261 | 43.664 | 1.00 | 58.04 |
| ATOM | 1927 | O | GLU | 279 | 40.196 | 1.346 | 43.298 | 1.00 | 59.38 |
| ATOM | 1928 | N | SER | 280 | 38.283 | 2.044 | 44.219 | 1.00 | 57.21 |
| ATOM | 1929 | CA | SER | 280 | 37.798 | 0.679 | 44.390 | 1.00 | 56.55 |
| ATOM | 1930 | CB | SER | 280 | 38.283 | 0.091 | 45.719 | 1.00 | 56.66 |
| ATOM | 1931 | OG | SER | 280 | 38.015 | −1.298 | 45.774 | 1.00 | 54.41 |
| ATOM | 1932 | C | SER | 280 | 36.282 | 0.671 | 44.334 | 1.00 | 55.29 |
| ATOM | 1933 | O | SER | 280 | 35.640 | −0.371 | 44.472 | 1.00 | 53.68 |
| ATOM | 1934 | N | SER | 281 | 35.725 | 1.854 | 44.113 | 1.00 | 54.58 |
| ATOM | 1935 | CA | SER | 281 | 34.288 | 2.038 | 44.020 | 1.00 | 55.36 |
| ATOM | 1936 | CB | SER | 281 | 33.919 | 3.451 | 44.464 | 1.00 | 56.89 |
| ATOM | 1937 | OG | SER | 281 | 34.565 | 4.415 | 43.649 | 1.00 | 56.89 |
| ATOM | 1938 | C | SER | 281 | 33.843 | 1.832 | 42.584 | 1.00 | 54.80 |
| ATOM | 1939 | O | SER | 281 | 34.652 | 1.905 | 41.664 | 1.00 | 55.85 |
| ATOM | 1940 | N | ALA | 282 | 32.553 | 1.587 | 42.389 | 1.00 | 53.75 |
| ATOM | 1941 | CA | ALA | 282 | 32.025 | 1.379 | 41.050 | 1.00 | 52.42 |
| ATOM | 1942 | CB | ALA | 282 | 30.626 | 0.809 | 41.133 | 1.00 | 52.26 |
| ATOM | 1943 | C | ALA | 282 | 32.012 | 2.679 | 40.250 | 1.00 | 51.83 |
| ATOM | 1944 | O | ALA | 282 | 31.632 | 2.685 | 39.081 | 1.00 | 52.27 |
| ATOM | 1945 | N | ASN | 283 | 32.441 | 3.772 | 40.879 | 1.00 | 50.19 |
| ATOM | 1946 | CA | ASN | 283 | 32.465 | 5.089 | 40.239 | 1.00 | 47.37 |
| ATOM | 1947 | CB | ASN | 283 | 31.338 | 5.945 | 40.790 | 1.00 | 47.04 |
| ATOM | 1948 | CG | ASN | 283 | 31.482 | 6.191 | 42.276 | 1.00 | 47.38 |
| ATOM | 1949 | OD1 | ASN | 283 | 31.584 | 5.255 | 43.068 | 1.00 | 46.86 |
| ATOM | 1950 | ND2 | ASN | 283 | 31.497 | 7.455 | 42.662 | 1.00 | 49.96 |
| ATOM | 1951 | C | ASN | 283 | 33.777 | 5.806 | 40.513 | 1.00 | 46.64 |
| ATOM | 1952 | O | ASN | 283 | 33.783 | 6.894 | 41.081 | 1.00 | 48.74 |
| ATOM | 1953 | N | PRO | 284 | 34.905 | 5.214 | 40.110 | 1.00 | 45.15 |
| ATOM | 1954 | CD | PRO | 284 | 35.028 | 3.896 | 39.462 | 1.00 | 44.41 |
| ATOM | 1955 | CA | PRO | 284 | 36.227 | 5.814 | 40.327 | 1.00 | 43.24 |
| ATOM | 1956 | CB | PRO | 284 | 37.151 | 4.855 | 39.583 | 1.00 | 44.66 |
| ATOM | 1957 | CG | PRO | 284 | 36.459 | 3.532 | 39.756 | 1.00 | 44.93 |
| ATOM | 1958 | C | PRO | 284 | 36.389 | 7.267 | 39.856 | 1.00 | 41.14 |
| ATOM | 1959 | O | PRO | 284 | 35.978 | 7.624 | 38.755 | 1.00 | 40.17 |
| ATOM | 1960 | N | GLY | 285 | 36.994 | 8.099 | 40.695 | 1.00 | 39.45 |
| ATOM | 1961 | CA | GLY | 285 | 37.208 | 9.484 | 40.321 | 1.00 | 40.34 |
| ATOM | 1962 | C | GLY | 285 | 35.964 | 10.343 | 40.401 | 1.00 | 42.06 |
| ATOM | 1963 | O | GLY | 285 | 36.035 | 11.576 | 40.367 | 1.00 | 43.11 |
| ATOM | 1964 | N | GLN | 286 | 34.811 | 9.699 | 40.510 | 1.00 | 42.34 |
| ATOM | 1965 | CA | GLN | 286 | 33.555 | 10.427 | 40.601 | 1.00 | 41.88 |
| ATOM | 1966 | CB | GLN | 286 | 32.490 | 9.717 | 39.758 | 1.00 | 44.97 |
| ATOM | 1967 | CG | GLN | 286 | 31.973 | 10.544 | 38.588 | 1.00 | 49.89 |
| ATOM | 1968 | CD | GLN | 286 | 31.043 | 11.668 | 39.043 | 1.00 | 54.72 |
| ATOM | 1969 | OE1 | GLN | 286 | 29.911 | 11.419 | 39.483 | 1.00 | 56.09 |
| ATOM | 1970 | NE2 | GLN | 286 | 31.519 | 12.911 | 38.950 | 1.00 | 54.20 |
| ATOM | 1971 | C | GLN | 286 | 33.113 | 10.541 | 42.063 | 1.00 | 40.59 |
| ATOM | 1972 | O | GLN | 286 | 33.396 | 9.660 | 42.879 | 1.00 | 39.39 |
| ATOM | 1973 | N | GLN | 287 | 32.445 | 11.648 | 42.389 | 1.00 | 39.59 |
| ATOM | 1974 | CA | GLN | 287 | 31.939 | 11.913 | 43.741 | 1.00 | 38.06 |
| ATOM | 1975 | CB | GLN | 287 | 30.770 | 10.969 | 44.053 | 1.00 | 37.29 |
| ATOM | 1976 | CG | GLN | 287 | 29.732 | 10.837 | 42.939 | 1.00 | 35.04 |
| ATOM | 1977 | CD | GLN | 287 | 28.912 | 12.100 | 42.736 | 1.00 | 33.74 |
| ATOM | 1978 | OE1 | GLN | 287 | 28.906 | 12.692 | 41.647 | 1.00 | 28.89 |
| ATOM | 1979 | NE2 | GLN | 287 | 28.204 | 12.514 | 43.786 | 1.00 | 31.49 |
| ATOM | 1980 | C | GLN | 287 | 33.015 | 11.744 | 44.820 | 1.00 | 37.30 |
| ATOM | 1981 | O | GLN | 287 | 32.958 | 10.813 | 45.624 | 1.00 | 37.53 |
| ATOM | 1982 | N | LEU | 288 | 33.990 | 12.643 | 44.856 | 1.00 | 34.03 |
| ATOM | 1983 | CA | LEU | 288 | 35.051 | 12.516 | 45.844 | 1.00 | 29.84 |
| ATOM | 1984 | CB | LEU | 288 | 36.351 | 13.071 | 45.293 | 1.00 | 30.50 |
| ATOM | 1985 | CG | LEU | 288 | 37.285 | 11.960 | 44.819 | 1.00 | 32.69 |
| ATOM | 1986 | CD1 | LEU | 288 | 36.645 | 11.102 | 43.728 | 1.00 | 31.90 |
| ATOM | 1987 | CD2 | LEU | 288 | 38.546 | 12.611 | 44.323 | 1.00 | 36.00 |
| ATOM | 1988 | C | LEU | 288 | 34.729 | 13.180 | 47.156 | 1.00 | 26.53 |
| ATOM | 1989 | O | LEU | 288 | 34.991 | 12.627 | 48.218 | 1.00 | 26.76 |
| ATOM | 1990 | N | TYR | 289 | 34.172 | 14.374 | 47.086 | 1.00 | 23.58 |
| ATOM | 1991 | CA | TYR | 289 | 33.809 | 15.074 | 48.292 | 1.00 | 22.36 |
| ATOM | 1992 | CB | TYR | 289 | 33.086 | 16.365 | 47.939 | 1.00 | 20.16 |
| ATOM | 1993 | CG | TYR | 289 | 32.716 | 17.186 | 49.136 | 1.00 | 18.61 |
| ATOM | 1994 | CD1 | TYR | 289 | 33.660 | 17.486 | 50.105 | 1.00 | 18.65 |
| ATOM | 1995 | CE1 | TYR | 289 | 33.347 | 18.269 | 51.195 | 1.00 | 18.34 |
| ATOM | 1996 | CD2 | TYR | 289 | 31.433 | 17.693 | 49.288 | 1.00 | 18.91 |
| ATOM | 1997 | CE2 | TYR | 289 | 31.105 | 18.484 | 50.378 | 1.00 | 18.97 |
| ATOM | 1998 | CZ | TYR | 289 | 32.073 | 18.768 | 51.327 | 1.00 | 20.15 |
| ATOM | 1999 | OH | TYR | 289 | 31.788 | 19.565 | 52.408 | 1.00 | 22.93 |
| ATOM | 2000 | C | TYR | 289 | 32.894 | 14.165 | 49.105 | 1.00 | 25.30 |
| ATOM | 2001 | O | TYR | 289 | 32.991 | 14.106 | 50.337 | 1.00 | 24.21 |
| ATOM | 2002 | N | GLU | 290 | 32.005 | 13.448 | 48.411 | 1.00 | 27.35 |
| ATOM | 2003 | CA | GLU | 290 | 31.071 | 12.532 | 49.084 | 1.00 | 26.68 |
| ATOM | 2004 | CB | GLU | 290 | 30.081 | 11.904 | 48.090 | 1.00 | 26.17 |
| ATOM | 2005 | CG | GLU | 290 | 28.614 | 12.216 | 48.413 | 1.00 | 25.68 |
| ATOM | 2006 | CD | GLU | 290 | 27.617 | 11.404 | 47.591 | 1.00 | 26.93 |
| ATOM | 2007 | OE1 | GLU | 290 | 27.735 | 11.363 | 46.337 | 1.00 | 22.27 |
| ATOM | 2008 | OE2 | GLU | 290 | 26.702 | 10.815 | 48.215 | 1.00 | 27.37 |
| ATOM | 2009 | C | GLU | 290 | 31.838 | 11.425 | 49.781 | 1.00 | 25.75 |
| ATOM | 2010 | O | GLU | 290 | 31.649 | 11.193 | 50.974 | 1.00 | 26.23 |
| ATOM | 2011 | N | LYS | 291 | 32.706 | 10.756 | 49.024 | 1.00 | 24.16 |
| ATOM | 2012 | CA | LYS | 291 | 33.526 | 9.666 | 49.538 | 1.00 | 24.45 |
| ATOM | 2013 | CB | LYS | 291 | 34.342 | 9.063 | 48.408 | 1.00 | 24.19 |
| ATOM | 2014 | CG | LYS | 291 | 33.506 | 8.383 | 47.354 | 1.00 | 28.37 |
| ATOM | 2015 | CD | LYS | 291 | 34.322 | 8.162 | 46.094 | 1.00 | 31.52 |
| ATOM | 2016 | CE | LYS | 291 | 33.533 | 7.434 | 45.030 | 1.00 | 31.16 |
| ATOM | 2017 | NZ | LYS | 291 | 34.367 | 7.299 | 43.813 | 1.00 | 33.55 |
| ATOM | 2018 | C | LYS | 291 | 34.460 | 10.143 | 50.636 | 1.00 | 24.99 |
| ATOM | 2019 | O | LYS | 291 | 35.488 | 9.522 | 50.918 | 1.00 | 25.78 |
| ATOM | 2020 | N | LEU | 292 | 34.095 | 11.254 | 51.255 | 1.00 | 24.20 |
| ATOM | 2021 | CA | LEU | 292 | 34.894 | 11.809 | 52.318 | 1.00 | 25.20 |
| ATOM | 2022 | CB | LEU | 292 | 35.544 | 13.106 | 51.843 | 1.00 | 25.62 |
| ATOM | 2023 | CG | LEU | 292 | 36.904 | 13.450 | 52.464 | 1.00 | 26.59 |
| ATOM | 2024 | CD1 | LEU | 292 | 37.935 | 12.396 | 52.035 | 1.00 | 26.37 |
| ATOM | 2025 | CD2 | LEU | 292 | 37.343 | 14.853 | 52.025 | 1.00 | 24.08 |
| ATOM | 2026 | C | LEU | 292 | 33.999 | 12.063 | 53.528 | 1.00 | 26.58 |
| ATOM | 2027 | O | LEU | 292 | 34.431 | 11.924 | 54.671 | 1.00 | 27.91 |
| ATOM | 2028 | N | ILE | 293 | 32.744 | 12.421 | 53.272 | 1.00 | 27.03 |
| ATOM | 2029 | CA | ILE | 293 | 31.783 | 12.689 | 54.342 | 1.00 | 26.01 |
| ATOM | 2030 | CB | ILE | 293 | 30.948 | 13.956 | 54.019 | 1.00 | 26.42 |
| ATOM | 2031 | CG2 | ILE | 293 | 30.184 | 14.431 | 55.247 | 1.00 | 25.08 |
| ATOM | 2032 | CG1 | ILE | 293 | 31.866 | 15.085 | 53.573 | 1.00 | 24.53 |
| ATOM | 2033 | CD1 | ILE | 293 | 31.131 | 16.366 | 53.336 | 1.00 | 23.77 |
| ATOM | 2034 | C | ILE | 293 | 30.827 | 11.503 | 54.489 | 1.00 | 24.65 |
| ATOM | 2035 | O | ILE | 293 | 30.681 | 10.919 | 55.565 | 1.00 | 23.84 |
| ATOM | 2036 | N | GLY | 294 | 30.197 | 11.159 | 53.374 | 1.00 | 24.02 |
| ATOM | 2037 | CA | GLY | 294 | 29.237 | 10.073 | 53.325 | 1.00 | 25.49 |
| ATOM | 2038 | C | GLY | 294 | 29.454 | 8.815 | 54.142 | 1.00 | 24.75 |
| ATOM | 2039 | O | GLY | 294 | 30.427 | 8.079 | 53.953 | 1.00 | 26.25 |
| ATOM | 2040 | N | GLY | 295 | 28.517 | 8.556 | 55.044 | 1.00 | 22.54 |
| ATOM | 2041 | CA | GLY | 295 | 28.607 | 7.369 | 55.851 | 1.00 | 22.80 |
| ATOM | 2042 | C | GLY | 295 | 28.530 | 6.125 | 54.986 | 1.00 | 25.08 |
| ATOM | 2043 | O | GLY | 295 | 28.252 | 5.047 | 55.497 | 1.00 | 27.80 |
| ATOM | 2044 | N | LYS | 296 | 28.748 | 6.238 | 53.680 | 1.00 | 25.43 |
| ATOM | 2045 | CA | LYS | 296 | 28.696 | 5.039 | 52.849 | 1.00 | 25.87 |
| ATOM | 2046 | CB | LYS | 296 | 28.313 | 5.354 | 51.411 | 1.00 | 27.04 |
| ATOM | 2047 | CG | LYS | 296 | 28.036 | 4.096 | 50.587 | 1.00 | 30.40 |
| ATOM | 2048 | CD | LYS | 296 | 29.249 | 3.562 | 49.842 | 1.00 | 30.20 |
| ATOM | 2049 | CE | LYS | 296 | 28.954 | 2.204 | 49.176 | 1.00 | 32.59 |
| ATOM | 2050 | NZ | LYS | 296 | 29.015 | 1.038 | 50.135 | 1.00 | 32.31 |
| ATOM | 2051 | C | LYS | 296 | 30.044 | 4.364 | 52.828 | 1.00 | 28.34 |
| ATOM | 2052 | O | LYS | 296 | 30.158 | 3.185 | 52.507 | 1.00 | 29.08 |
| ATOM | 2053 | N | TYR | 297 | 31.075 | 5.122 | 53.163 | 1.00 | 29.56 |
| ATOM | 2054 | CA | TYR | 297 | 32.414 | 4.582 | 53.147 | 1.00 | 29.25 |
| ATOM | 2055 | CB | TYR | 297 | 33.208 | 5.230 | 52.022 | 1.00 | 30.07 |
| ATOM | 2056 | CG | TYR | 297 | 32.620 | 5.025 | 50.650 | 1.00 | 30.84 |
| ATOM | 2057 | CD1 | TYR | 297 | 32.023 | 6.082 | 49.960 | 1.00 | 32.45 |
| ATOM | 2058 | CE1 | TYR | 297 | 31.544 | 5.915 | 48.665 | 1.00 | 35.21 |
| ATOM | 2059 | CD2 | TYR | 297 | 32.715 | 3.789 | 50.015 | 1.00 | 30.51 |
| ATOM | 2060 | CE2 | TYR | 297 | 32.244 | 3.604 | 48.724 | 1.00 | 34.82 |
| ATOM | 2061 | CZ | TYR | 297 | 31.661 | 4.673 | 48.049 | 1.00 | 37.82 |
| ATOM | 2062 | OH | TYR | 297 | 31.219 | 4.504 | 46.753 | 1.00 | 41.74 |
| ATOM | 2063 | C | TYR | 297 | 33.097 | 4.842 | 54.465 | 1.00 | 27.53 |
| ATOM | 2064 | O | TYR | 297 | 34.174 | 4.312 | 54.731 | 1.00 | 28.35 |
| ATOM | 2065 | N | MET | 298 | 32.464 | 5.665 | 55.288 | 1.00 | 24.45 |
| ATOM | 2066 | CA | MET | 298 | 33.025 | 6.000 | 56.580 | 1.00 | 23.96 |
| ATOM | 2067 | CB | MET | 298 | 31.959 | 6.652 | 57.454 | 1.00 | 21.69 |
| ATOM | 2068 | CG | MET | 298 | 32.436 | 6.992 | 58.850 | 1.00 | 20.73 |
| ATOM | 2069 | SD | MET | 298 | 31.288 | 8.100 | 59.701 | 1.00 | 20.68 |
| ATOM | 2070 | CE | MET | 298 | 31.435 | 9.523 | 58.620 | 1.00 | 18.32 |
| ATOM | 2071 | C | MET | 298 | 33.579 | 4.750 | 57.254 | 1.00 | 24.25 |
| ATOM | 2072 | O | MET | 298 | 34.776 | 4.656 | 57.529 | 1.00 | 24.74 |
| ATOM | 2073 | N | GLY | 299 | 32.707 | 3.779 | 57.494 | 1.00 | 26.72 |
| ATOM | 2074 | CA | GLY | 299 | 33.135 | 2.552 | 58.135 | 1.00 | 25.77 |
| ATOM | 2075 | C | GLY | 299 | 34.301 | 1.906 | 57.424 | 1.00 | 25.50 |
| ATOM | 2076 | O | GLY | 299 | 35.162 | 1.331 | 58.076 | 1.00 | 26.16 |
| ATOM | 2077 | N | GLU | 300 | 34.325 | 2.004 | 56.095 | 1.00 | 25.37 |
| ATOM | 2078 | CA | GLU | 300 | 35.389 | 1.418 | 55.282 | 1.00 | 24.57 |
| ATOM | 2079 | CB | GLU | 300 | 35.057 | 1.551 | 53.800 | 1.00 | 24.05 |
| ATOM | 2080 | CG | GLU | 300 | 36.066 | 0.859 | 52.905 | 1.00 | 24.66 |
| ATOM | 2081 | CD | GLU | 300 | 36.018 | −0.662 | 53.004 | 1.00 | 24.52 |
| ATOM | 2082 | OE1 | GLU | 300 | 35.581 | −1.195 | 54.054 | 1.00 | 24.02 |
| ATOM | 2083 | OE2 | GLU | 300 | 36.438 | −1.319 | 52.026 | 1.00 | 22.70 |
| ATOM | 2084 | C | GLU | 300 | 36.734 | 2.082 | 55.550 | 1.00 | 25.31 |
| ATOM | 2085 | O | GLU | 300 | 37.769 | 1.408 | 55.663 | 1.00 | 22.71 |
| ATOM | 2086 | N | LEU | 301 | 36.712 | 3.409 | 55.622 | 1.00 | 26.47 |
| ATOM | 2087 | CA | LEU | 301 | 37.919 | 4.174 | 55.900 | 1.00 | 26.65 |
| ATOM | 2088 | CB | LEU | 301 | 37.600 | 5.676 | 55.992 | 1.00 | 26.57 |
| ATOM | 2089 | CG | LEU | 301 | 37.165 | 6.395 | 54.701 | 1.00 | 26.02 |
| ATOM | 2090 | CD1 | LEU | 301 | 36.684 | 7.784 | 55.047 | 1.00 | 27.06 |
| ATOM | 2091 | CD2 | LEU | 301 | 38.312 | 6.474 | 53.701 | 1.00 | 25.38 |
| ATOM | 2092 | C | LEU | 301 | 38.452 | 3.648 | 57.226 | 1.00 | 26.23 |
| ATOM | 2093 | O | LEU | 301 | 39.594 | 3.209 | 57.313 | 1.00 | 26.97 |
| ATOM | 2094 | N | VAL | 302 | 37.623 | 3.670 | 58.259 | 1.00 | 26.05 |
| ATOM | 2095 | CA | VAL | 302 | 38.068 | 3.154 | 59.542 | 1.00 | 27.56 |
| ATOM | 2096 | CB | VAL | 302 | 36.911 | 3.034 | 60.524 | 1.00 | 28.13 |
| ATOM | 2097 | CG1 | VAL | 302 | 37.354 | 2.285 | 61.777 | 1.00 | 26.62 |
| ATOM | 2098 | CG2 | VAL | 302 | 36.433 | 4.424 | 60.882 | 1.00 | 30.95 |
| ATOM | 2099 | C | VAL | 302 | 38.723 | 1.786 | 59.386 | 1.00 | 27.42 |
| ATOM | 2100 | O | VAL | 302 | 39.765 | 1.529 | 59.977 | 1.00 | 29.00 |
| ATOM | 2101 | N | ARG | 303 | 38.127 | 0.906 | 58.593 | 1.00 | 25.04 |
| ATOM | 2102 | CA | ARG | 303 | 38.723 | −0.395 | 58.417 | 1.00 | 25.12 |
| ATOM | 2103 | CB | ARG | 303 | 37.906 | −1.254 | 57.475 | 1.00 | 26.51 |
| ATOM | 2104 | CG | ARG | 303 | 38.587 | −2.558 | 57.126 | 1.00 | 28.11 |
| ATOM | 2105 | CD | ARG | 303 | 37.609 | −3.520 | 56.490 | 1.00 | 31.77 |
| ATOM | 2106 | NE | ARG | 303 | 38.260 | −4.456 | 55.583 | 1.00 | 32.46 |
| ATOM | 2107 | CZ | ARG | 303 | 38.483 | −4.215 | 54.296 | 1.00 | 34.64 |
| ATOM | 2108 | NH1 | ARG | 303 | 38.103 | −3.059 | 53.759 | 1.00 | 33.51 |
| ATOM | 2109 | NH2 | ARG | 303 | 39.082 | −5.136 | 53.546 | 1.00 | 35.80 |
| ATOM | 2110 | C | ARG | 303 | 40.111 | −0.242 | 57.854 | 1.00 | 27.77 |
| ATOM | 2111 | O | ARG | 303 | 41.073 | −0.788 | 58.401 | 1.00 | 30.47 |
| ATOM | 2112 | N | LEU | 304 | 40.236 | 0.495 | 56.754 | 1.00 | 27.67 |
| ATOM | 2113 | CA | LEU | 304 | 41.562 | 0.674 | 56.147 | 1.00 | 24.93 |
| ATOM | 2114 | CB | LEU | 304 | 41.464 | 1.526 | 54.865 | 1.00 | 22.51 |
| ATOM | 2115 | CG | LEU | 304 | 40.640 | 0.902 | 53.718 | 1.00 | 19.14 |
| ATOM | 2116 | CD1 | LEU | 304 | 40.386 | 1.957 | 52.675 | 1.00 | 19.15 |
| ATOM | 2117 | CD2 | LEU | 304 | 41.352 | −0.295 | 53.105 | 1.00 | 14.45 |
| ATOM | 2118 | C | LEU | 304 | 42.523 | 1.290 | 57.168 | 1.00 | 21.35 |
| ATOM | 2119 | O | LEU | 304 | 43.584 | 0.736 | 57.432 | 1.00 | 20.90 |
| ATOM | 2120 | N | VAL | 305 | 42.142 | 2.406 | 57.770 | 1.00 | 17.52 |
| ATOM | 2121 | CA | VAL | 305 | 43.003 | 3.011 | 58.758 | 1.00 | 17.43 |
| ATOM | 2122 | CB | VAL | 305 | 42.316 | 4.162 | 59.423 | 1.00 | 14.40 |
| ATOM | 2123 | CG1 | VAL | 305 | 43.154 | 4.673 | 60.583 | 1.00 | 14.53 |
| ATOM | 2124 | CG2 | VAL | 305 | 42.095 | 5.240 | 58.408 | 1.00 | 14.33 |
| ATOM | 2125 | C | VAL | 305 | 43.400 | 2.010 | 59.829 | 1.00 | 20.92 |
| ATOM | 2126 | O | VAL | 305 | 44.497 | 2.071 | 60.387 | 1.00 | 22.69 |
| ATOM | 2127 | N | LEU | 306 | 42.502 | 1.085 | 60.126 | 1.00 | 24.02 |
| ATOM | 2128 | CA | LEU | 306 | 42.783 | 0.081 | 61.144 | 1.00 | 26.64 |
| ATOM | 2129 | CB | LEU | 306 | 41.481 | −0.585 | 61.594 | 1.00 | 27.02 |
| ATOM | 2130 | CG | LEU | 306 | 41.154 | −0.563 | 63.087 | 1.00 | 27.64 |
| ATOM | 2131 | CD1 | LEU | 306 | 41.094 | 0.873 | 63.592 | 1.00 | 27.51 |
| ATOM | 2132 | CD2 | LEU | 306 | 39.826 | −1.267 | 63.311 | 1.00 | 28.07 |
| ATOM | 2133 | C | LEU | 306 | 43.721 | −0.965 | 60.566 | 1.00 | 27.73 |
| ATOM | 2134 | O | LEU | 306 | 44.745 | −1.303 | 61.157 | 1.00 | 26.86 |
| ATOM | 2135 | N | LEU | 307 | 43.360 | −1.467 | 59.394 | 1.00 | 28.77 |
| ATOM | 2136 | CA | LEU | 307 | 44.156 | −2.478 | 58.733 | 1.00 | 32.47 |
| ATOM | 2137 | CB | LEU | 307 | 43.465 | −2.893 | 57.437 | 1.00 | 29.90 |
| ATOM | 2138 | CG | LEU | 307 | 43.477 | −4.392 | 57.130 | 1.00 | 29.19 |
| ATOM | 2139 | CD1 | LEU | 307 | 43.104 | −5.210 | 58.361 | 1.00 | 28.38 |
| ATOM | 2140 | CD2 | LEU | 307 | 42.495 | −4.648 | 56.015 | 1.00 | 29.88 |
| ATOM | 2141 | C | LEU | 307 | 45.553 | −1.916 | 58.466 | 1.00 | 35.49 |
| ATOM | 2142 | O | LEU | 307 | 46.542 | −2.645 | 58.394 | 1.00 | 36.50 |
| ATOM | 2143 | N | ARG | 308 | 45.622 | −0.602 | 58.332 | 1.00 | 38.03 |
| ATOM | 2144 | CA | ARG | 308 | 46.882 | 0.080 | 58.101 | 1.00 | 41.29 |
| ATOM | 2145 | CB | ARG | 308 | 46.603 | 1.580 | 57.936 | 1.00 | 47.88 |
| ATOM | 2146 | CG | ARG | 308 | 47.706 | 2.544 | 58.368 | 1.00 | 54.88 |
| ATOM | 2147 | CD | ARG | 308 | 48.819 | 2.693 | 57.338 | 1.00 | 60.14 |
| ATOM | 2148 | NE | ARG | 308 | 49.524 | 3.958 | 57.540 | 1.00 | 65.47 |
| ATOM | 2149 | CZ | ARG | 308 | 50.523 | 4.401 | 56.784 | 1.00 | 67.54 |
| ATOM | 2150 | NH1 | ARG | 308 | 50.954 | 3.673 | 55.757 | 1.00 | 68.57 |
| ATOM | 2151 | NH2 | ARG | 308 | 51.074 | 5.584 | 57.046 | 1.00 | 66.83 |
| ATOM | 2152 | C | ARG | 308 | 47.783 | −0.182 | 59.301 | 1.00 | 40.42 |
| ATOM | 2153 | O | ARG | 308 | 48.889 | −0.694 | 59.159 | 1.00 | 40.04 |
| ATOM | 2154 | N | LEU | 309 | 47.287 | 0.152 | 60.487 | 1.00 | 39.27 |
| ATOM | 2155 | CA | LEU | 309 | 48.043 | −0.027 | 61.717 | 1.00 | 38.92 |
| ATOM | 2156 | CB | LEU | 309 | 47.224 | 0.484 | 62.895 | 1.00 | 33.74 |
| ATOM | 2157 | CG | LEU | 309 | 46.852 | 1.958 | 62.854 | 1.00 | 30.26 |
| ATOM | 2158 | CD1 | LEU | 309 | 45.453 | 2.121 | 63.368 | 1.00 | 30.84 |
| ATOM | 2159 | CD2 | LEU | 309 | 47.819 | 2.766 | 63.683 | 1.00 | 27.57 |
| ATOM | 2160 | C | LEU | 309 | 48.461 | −1.473 | 61.984 | 1.00 | 41.92 |
| ATOM | 2161 | O | LEU | 309 | 49.600 | −1.741 | 62.364 | 1.00 | 42.73 |
| ATOM | 2162 | N | VAL | 310 | 47.541 | −2.406 | 61.788 | 1.00 | 44.59 |
| ATOM | 2163 | CA | VAL | 310 | 47.829 | −3.811 | 62.039 | 1.00 | 46.67 |
| ATOM | 2164 | CB | VAL | 310 | 46.606 | −4.651 | 61.798 | 1.00 | 46.95 |
| ATOM | 2165 | CG1 | VAL | 310 | 45.419 | −4.006 | 62.479 | 1.00 | 49.54 |
| ATOM | 2166 | CG2 | VAL | 310 | 46.368 | −4.779 | 60.312 | 1.00 | 47.77 |
| ATOM | 2167 | C | VAL | 310 | 48.929 | −4.321 | 61.139 | 1.00 | 47.55 |
| ATOM | 2168 | O | VAL | 310 | 49.488 | −5.392 | 61.374 | 1.00 | 48.66 |
| ATOM | 2169 | N | ASP | 311 | 49.217 | −3.559 | 60.093 | 1.00 | 48.93 |
| ATOM | 2170 | CA | ASP | 311 | 50.262 | −3.927 | 59.160 | 1.00 | 52.04 |
| ATOM | 2171 | CB | ASP | 311 | 49.993 | −3.298 | 57.793 | 1.00 | 57.14 |
| ATOM | 2172 | CG | ASP | 311 | 48.752 | −3.869 | 57.135 | 1.00 | 61.79 |
| ATOM | 2173 | OD1 | ASP | 311 | 48.348 | −3.377 | 56.054 | 1.00 | 63.59 |
| ATOM | 2174 | OD2 | ASP | 311 | 48.180 | −4.819 | 57.713 | 1.00 | 63.98 |
| ATOM | 2175 | C | ASP | 311 | 51.618 | −3.490 | 59.698 | 1.00 | 51.94 |
| ATOM | 2176 | O | ASP | 311 | 52.580 | −4.256 | 59.653 | 1.00 | 53.89 |
| ATOM | 2177 | N | GLU | 312 | 51.702 | −2.267 | 60.212 | 1.00 | 49.51 |
| ATOM | 2178 | CA | GLU | 312 | 52.961 | −1.785 | 60.762 | 1.00 | 47.68 |
| ATOM | 2179 | CB | GLU | 312 | 53.071 | −0.272 | 60.632 | 1.00 | 48.44 |
| ATOM | 2180 | CG | GLU | 312 | 52.900 | 0.221 | 59.216 | 1.00 | 51.79 |
| ATOM | 2181 | CD | GLU | 312 | 53.122 | 1.713 | 59.084 | 1.00 | 53.56 |
| ATOM | 2182 | OE1 | GLU | 312 | 52.698 | 2.280 | 58.047 | 1.00 | 49.90 |
| ATOM | 2183 | OE2 | GLU | 312 | 53.725 | 2.309 | 60.013 | 1.00 | 56.82 |
| ATOM | 2184 | C | GLU | 312 | 53.075 | −2.172 | 62.222 | 1.00 | 46.11 |
| ATOM | 2185 | O | GLU | 312 | 53.514 | −1.377 | 63.049 | 1.00 | 46.75 |
| ATOM | 2186 | N | ASN | 313 | 52.666 | −3.397 | 62.527 | 1.00 | 45.02 |
| ATOM | 2187 | CA | ASN | 313 | 52.720 | −3.938 | 63.879 | 1.00 | 44.64 |
| ATOM | 2188 | CB | ASN | 313 | 54.100 | −4.550 | 64.119 | 1.00 | 43.84 |
| ATOM | 2189 | CG | ASN | 313 | 54.028 | −5.860 | 64.863 | 1.00 | 45.16 |
| ATOM | 2190 | OD1 | ASN | 313 | 53.377 | −5.965 | 65.906 | 1.00 | 43.79 |
| ATOM | 2191 | ND2 | ASN | 313 | 54.701 | −6.875 | 64.333 | 1.00 | 46.05 |
| ATOM | 2192 | C | ASN | 313 | 52.408 | −2.921 | 64.991 | 1.00 | 44.49 |
| ATOM | 2193 | O | ASN | 313 | 53.303 | −2.509 | 65.728 | 1.00 | 45.19 |
| ATOM | 2194 | N | LEU | 314 | 51.142 | −2.530 | 65.126 | 1.00 | 43.02 |
| ATOM | 2195 | CA | LEU | 314 | 50.743 | −1.563 | 66.159 | 1.00 | 40.80 |
| ATOM | 2196 | CB | LEU | 314 | 50.639 | −0.167 | 65.549 | 1.00 | 34.97 |
| ATOM | 2197 | CG | LEU | 314 | 51.940 | 0.499 | 65.127 | 1.00 | 29.58 |
| ATOM | 2198 | CD1 | LEU | 314 | 51.698 | 1.453 | 63.981 | 1.00 | 28.94 |
| ATOM | 2199 | CD2 | LEU | 314 | 52.516 | 1.212 | 66.311 | 1.00 | 28.16 |
| ATOM | 2200 | C | LEU | 314 | 49.396 | −1.924 | 66.777 | 1.00 | 42.38 |
| ATOM | 2201 | O | LEU | 314 | 49.026 | −1.422 | 67.848 | 1.00 | 39.73 |
| ATOM | 2202 | N | LEU | 315 | 48.689 | −2.812 | 66.078 | 1.00 | 44.49 |
| ATOM | 2203 | CA | LEU | 315 | 47.352 | −3.268 | 66.439 | 1.00 | 45.22 |
| ATOM | 2204 | CB | LEU | 315 | 46.354 | −2.695 | 65.445 | 1.00 | 43.49 |
| ATOM | 2205 | CG | LEU | 315 | 45.121 | −2.063 | 66.045 | 1.00 | 43.28 |
| ATOM | 2206 | CD1 | LEU | 315 | 44.055 | −1.976 | 64.972 | 1.00 | 43.01 |
| ATOM | 2207 | CD2 | LEU | 315 | 44.643 | −2.907 | 67.209 | 1.00 | 46.13 |
| ATOM | 2208 | C | LEU | 315 | 47.214 | −4.781 | 66.407 | 1.00 | 46.34 |
| ATOM | 2209 | O | LEU | 315 | 47.828 | −5.439 | 65.577 | 1.00 | 47.74 |
| ATOM | 2210 | N | PHE | 316 | 46.380 | −5.318 | 67.292 | 1.00 | 48.50 |
| ATOM | 2211 | CA | PHE | 316 | 46.125 | −6.760 | 67.369 | 1.00 | 50.80 |
| ATOM | 2212 | CB | PHE | 316 | 45.054 | −7.186 | 66.347 | 1.00 | 48.89 |
| ATOM | 2213 | CG | PHE | 316 | 43.829 | −6.312 | 66.331 | 1.00 | 46.47 |
| ATOM | 2214 | CD1 | PHE | 316 | 43.163 | −5.999 | 67.508 | 1.00 | 45.93 |
| ATOM | 2215 | CD2 | PHE | 316 | 43.350 | −5.791 | 65.134 | 1.00 | 44.48 |
| ATOM | 2216 | CE1 | PHE | 316 | 42.043 | −5.183 | 67.491 | 1.00 | 44.57 |
| ATOM | 2217 | CE2 | PHE | 316 | 42.229 | −4.974 | 65.109 | 1.00 | 43.59 |
| ATOM | 2218 | CZ | PHE | 316 | 41.577 | −4.669 | 66.290 | 1.00 | 44.05 |
| ATOM | 2219 | C | PHE | 316 | 47.371 | −7.605 | 67.124 | 1.00 | 53.06 |
| ATOM | 2220 | O | PHE | 316 | 47.342 | −8.521 | 66.299 | 1.00 | 54.62 |
| ATOM | 2221 | N | HIS | 317 | 48.456 | −7.304 | 67.835 | 1.00 | 54.60 |
| ATOM | 2222 | CA | HIS | 317 | 49.710 | −8.046 | 67.691 | 1.00 | 55.95 |
| ATOM | 2223 | CB | HIS | 317 | 49.676 | −9.301 | 68.569 | 1.00 | 54.90 |
| ATOM | 2224 | CG | HIS | 317 | 49.708 | −9.004 | 70.034 | 1.00 | 55.21 |
| ATOM | 2225 | CD2 | HIS | 317 | 49.686 | −9.823 | 71.113 | 1.00 | 55.22 |
| ATOM | 2226 | ND1 | HIS | 317 | 49.778 | −7.718 | 70.528 | 1.00 | 54.55 |
| ATOM | 2227 | CE1 | HIS | 317 | 49.798 | −7.756 | 71.848 | 1.00 | 55.21 |
| ATOM | 2228 | NE2 | HIS | 317 | 49.744 | −9.020 | 72.229 | 1.00 | 56.90 |
| ATOM | 2229 | C | HIS | 317 | 50.004 | −8.426 | 66.240 | 1.00 | 58.27 |
| ATOM | 2230 | O | HIS | 317 | 50.521 | −9.514 | 65.950 | 1.00 | 58.90 |
| ATOM | 2231 | N | GLY | 318 | 49.665 | −7.513 | 65.335 | 1.00 | 59.86 |
| ATOM | 2232 | CA | GLY | 318 | 49.881 | −7.734 | 63.921 | 1.00 | 60.72 |
| ATOM | 2233 | C | GLY | 318 | 49.290 | −9.022 | 63.379 | 1.00 | 62.25 |
| ATOM | 2234 | O | GLY | 318 | 50.031 | −9.956 | 63.080 | 1.00 | 63.75 |
| ATOM | 2235 | N | GLU | 319 | 47.962 | −9.087 | 63.277 | 1.00 | 62.86 |
| ATOM | 2236 | CA | GLU | 319 | 47.277 | −10.257 | 62.716 | 1.00 | 62.72 |
| ATOM | 2237 | CB | GLU | 319 | 47.663 | −11.545 | 63.439 | 1.00 | 66.93 |
| ATOM | 2238 | CG | GLU | 319 | 47.437 | −12.784 | 62.575 | 1.00 | 73.23 |
| ATOM | 2239 | CD | GLU | 319 | 47.862 | −14.068 | 63.262 | 1.00 | 78.58 |
| ATOM | 2240 | OE1 | GLU | 319 | 49.020 | −14.129 | 63.745 | 1.00 | 80.57 |
| ATOM | 2241 | OE2 | GLU | 319 | 47.043 | −15.019 | 63.310 | 1.00 | 81.49 |
| ATOM | 2242 | C | GLU | 319 | 45.765 | −10.097 | 62.739 | 1.00 | 59.42 |
| ATOM | 2243 | O | GLU | 319 | 45.098 | −10.387 | 63.735 | 1.00 | 57.03 |
| ATOM | 2244 | N | ALA | 320 | 45.246 | −9.643 | 61.604 | 1.00 | 55.74 |
| ATOM | 2245 | CA | ALA | 320 | 43.828 | −9.394 | 61.414 | 1.00 | 54.02 |
| ATOM | 2246 | CB | ALA | 320 | 43.657 | −8.357 | 60.338 | 1.00 | 52.55 |
| ATOM | 2247 | C | ALA | 320 | 43.052 | −10.650 | 61.043 | 1.00 | 54.49 |
| ATOM | 2248 | O | ALA | 320 | 43.620 | −11.565 | 60.457 | 1.00 | 55.61 |
| ATOM | 2249 | N | SER | 321 | 41.762 | −10.698 | 61.388 | 1.00 | 55.01 |
| ATOM | 2250 | CA | SER | 321 | 40.924 | −11.856 | 61.050 | 1.00 | 55.90 |
| ATOM | 2251 | CB | SER | 321 | 39.649 | −11.911 | 61.895 | 1.00 | 56.08 |
| ATOM | 2252 | OG | SER | 321 | 38.814 | −12.975 | 61.445 | 1.00 | 53.96 |
| ATOM | 2253 | C | SER | 321 | 40.513 | −11.780 | 59.589 | 1.00 | 55.49 |
| ATOM | 2254 | O | SER | 321 | 40.367 | −10.689 | 59.041 | 1.00 | 54.92 |
| ATOM | 2255 | N | GLU | 322 | 40.292 | −12.933 | 58.967 | 1.00 | 54.84 |
| ATOM | 2256 | CA | GLU | 322 | 39.917 | −12.951 | 57.563 | 1.00 | 56.14 |
| ATOM | 2257 | CB | GLU | 322 | 39.646 | −14.382 | 57.092 | 1.00 | 58.38 |
| ATOM | 2258 | CG | GLU | 322 | 40.173 | −14.697 | 55.681 | 1.00 | 63.26 |
| ATOM | 2259 | CD | GLU | 322 | 41.712 | −14.670 | 55.574 | 1.00 | 66.36 |
| ATOM | 2260 | OE1 | GLU | 322 | 42.296 | −13.571 | 55.432 | 1.00 | 66.15 |
| ATOM | 2261 | OE2 | GLU | 322 | 42.339 | −15.754 | 55.637 | 1.00 | 66.78 |
| ATOM | 2262 | C | GLU | 322 | 38.685 | −12.085 | 57.354 | 1.00 | 55.71 |
| ATOM | 2263 | O | GLU | 322 | 38.343 | −11.727 | 56.227 | 1.00 | 54.93 |
| ATOM | 2264 | N | GLN | 323 | 38.027 | −11.740 | 58.454 | 1.00 | 55.82 |
| ATOM | 2265 | CA | GLN | 323 | 36.838 | −10.904 | 58.393 | 1.00 | 55.20 |
| ATOM | 2266 | CB | GLN | 323 | 35.995 | −11.101 | 59.659 | 1.00 | 57.22 |
| ATOM | 2267 | CG | GLN | 323 | 35.737 | −12.571 | 59.983 | 1.00 | 60.42 |
| ATOM | 2268 | CD | GLN | 323 | 34.801 | −12.778 | 61.164 | 1.00 | 62.11 |
| ATOM | 2269 | OE1 | GLN | 323 | 34.596 | −13.909 | 61.612 | 1.00 | 63.58 |
| ATOM | 2270 | NE2 | GLN | 323 | 34.223 | −11.690 | 61.668 | 1.00 | 61.37 |
| ATOM | 2271 | C | GLN | 323 | 37.259 | −9.445 | 58.249 | 1.00 | 53.59 |
| ATOM | 2272 | O | GLN | 323 | 36.963 | −8.800 | 57.242 | 1.00 | 53.27 |
| ATOM | 2273 | N | LEU | 324 | 37.973 | −8.936 | 59.248 | 1.00 | 50.98 |
| ATOM | 2274 | CA | LEU | 324 | 38.430 | −7.553 | 59.224 | 1.00 | 48.40 |
| ATOM | 2275 | CB | LEU | 324 | 39.396 | −7.294 | 60.378 | 1.00 | 46.63 |
| ATOM | 2276 | CG | LEU | 324 | 39.956 | −5.876 | 60.498 | 1.00 | 44.87 |
| ATOM | 2277 | CD1 | LEU | 324 | 38.846 | −4.837 | 60.390 | 1.00 | 44.21 |
| ATOM | 2278 | CD2 | LEU | 324 | 40.671 | −5.758 | 61.827 | 1.00 | 43.22 |
| ATOM | 2279 | C | LEU | 324 | 39.115 | −7.224 | 57.911 | 1.00 | 47.25 |
| ATOM | 2280 | O | LEU | 324 | 39.181 | −6.065 | 57.505 | 1.00 | 44.86 |
| ATOM | 2281 | N | ARG | 325 | 39.627 | −8.253 | 57.252 | 1.00 | 48.35 |
| ATOM | 2282 | CA | ARG | 325 | 40.309 | −8.057 | 55.988 | 1.00 | 50.22 |
| ATOM | 2283 | CB | ARG | 325 | 41.473 | −9.055 | 55.839 | 1.00 | 53.47 |
| ATOM | 2284 | CG | ARG | 325 | 42.580 | −8.896 | 56.894 | 1.00 | 57.97 |
| ATOM | 2285 | CD | ARG | 325 | 43.660 | −9.986 | 56.808 | 1.00 | 61.92 |
| ATOM | 2286 | NE | ARG | 325 | 44.564 | −9.957 | 57.966 | 1.00 | 67.95 |
| ATOM | 2287 | CZ | ARG | 325 | 45.535 | −10.844 | 58.206 | 1.00 | 70.27 |
| ATOM | 2288 | NH1 | ARG | 325 | 45.753 | −11.854 | 57.371 | 1.00 | 69.69 |
| ATOM | 2289 | NH2 | ARG | 325 | 46.290 | −10.725 | 59.293 | 1.00 | 70.39 |
| ATOM | 2290 | C | ARG | 325 | 39.320 | −8.224 | 54.850 | 1.00 | 48.80 |
| ATOM | 2291 | O | ARG | 325 | 39.617 | −8.859 | 53.847 | 1.00 | 50.46 |
| ATOM | 2292 | N | THR | 326 | 38.131 | −7.663 | 54.999 | 1.00 | 46.54 |
| ATOM | 2293 | CA | THR | 326 | 37.162 | −7.783 | 53.929 | 1.00 | 45.13 |
| ATOM | 2294 | CB | THR | 326 | 36.108 | −8.810 | 54.264 | 1.00 | 44.85 |
| ATOM | 2295 | OG1 | THR | 326 | 36.749 | −10.061 | 54.546 | 1.00 | 44.98 |
| ATOM | 2296 | CG2 | THR | 326 | 35.160 | −8.973 | 53.092 | 1.00 | 43.46 |
| ATOM | 2297 | C | THR | 326 | 36.500 | −6.453 | 53.687 | 1.00 | 44.79 |
| ATOM | 2298 | O | THR | 326 | 36.256 | −5.705 | 54.626 | 1.00 | 45.01 |
| ATOM | 2299 | N | ARG | 327 | 36.216 | −6.143 | 52.430 | 1.00 | 45.02 |
| ATOM | 2300 | CA | ARG | 327 | 35.590 | −4.866 | 52.136 | 1.00 | 45.97 |
| ATOM | 2301 | CB | ARG | 327 | 35.476 | −4.655 | 50.623 | 1.00 | 48.63 |
| ATOM | 2302 | CG | ARG | 327 | 34.961 | −3.283 | 50.229 | 1.00 | 53.97 |
| ATOM | 2303 | CD | ARG | 327 | 34.975 | −3.072 | 48.722 | 1.00 | 58.44 |
| ATOM | 2304 | NE | ARG | 327 | 33.747 | −2.410 | 48.282 | 1.00 | 66.14 |
| ATOM | 2305 | CZ | ARG | 327 | 33.387 | −1.178 | 48.648 | 1.00 | 69.53 |
| ATOM | 2306 | NH1 | ARG | 327 | 34.167 | −0.471 | 49.458 | 1.00 | 69.84 |
| ATOM | 2307 | NH2 | ARG | 327 | 32.242 | −0.652 | 48.220 | 1.00 | 68.29 |
| ATOM | 2308 | C | ARG | 327 | 34.217 | −4.790 | 52.794 | 1.00 | 44.69 |
| ATOM | 2309 | O | ARG | 327 | 33.486 | −5.784 | 52.861 | 1.00 | 44.55 |
| ATOM | 2310 | N | GLY | 328 | 33.888 | −3.605 | 53.302 | 1.00 | 42.14 |
| ATOM | 2311 | CA | GLY | 328 | 32.606 | −3.394 | 53.952 | 1.00 | 37.48 |
| ATOM | 2312 | C | GLY | 328 | 32.480 | −4.007 | 55.334 | 1.00 | 33.00 |
| ATOM | 2313 | O | GLY | 328 | 31.693 | −3.532 | 56.148 | 1.00 | 32.88 |
| ATOM | 2314 | N | ALA | 329 | 33.258 | −5.049 | 55.601 | 1.00 | 29.02 |
| ATOM | 2315 | CA | ALA | 329 | 33.227 | −5.743 | 56.885 | 1.00 | 26.22 |
| ATOM | 2316 | CB | ALA | 329 | 34.452 | −6.623 | 57.028 | 1.00 | 28.65 |
| ATOM | 2317 | C | ALA | 329 | 33.092 | −4.861 | 58.115 | 1.00 | 24.38 |
| ATOM | 2318 | O | ALA | 329 | 32.490 | −5.276 | 59.097 | 1.00 | 26.43 |
| ATOM | 2319 | N | PHE | 330 | 33.663 | −3.663 | 58.091 | 1.00 | 21.81 |
| ATOM | 2320 | CA | PHE | 330 | 33.547 | −2.776 | 59.242 | 1.00 | 18.07 |
| ATOM | 2321 | CB | PHE | 330 | 34.887 | −2.137 | 59.558 | 1.00 | 13.90 |
| ATOM | 2322 | CG | PHE | 330 | 34.913 | −1.404 | 60.862 | 1.00 | 12.45 |
| ATOM | 2323 | CD1 | PHE | 330 | 34.460 | −0.096 | 60.961 | 1.00 | 12.64 |
| ATOM | 2324 | CD2 | PHE | 330 | 35.436 | −2.009 | 61.995 | 1.00 | 12.73 |
| ATOM | 2325 | CE1 | PHE | 330 | 34.535 | 0.605 | 62.188 | 1.00 | 12.83 |
| ATOM | 2326 | CE2 | PHE | 330 | 35.515 | −1.315 | 63.221 | 1.00 | 11.49 |
| ATOM | 2327 | CZ | PHE | 330 | 35.066 | −0.008 | 63.315 | 1.00 | 8.96 |
| ATOM | 2328 | C | PHE | 330 | 32.528 | −1.716 | 58.886 | 1.00 | 17.48 |
| ATOM | 2329 | O | PHE | 330 | 32.855 | −0.702 | 58.273 | 1.00 | 17.97 |
| ATOM | 2330 | N | GLU | 331 | 31.288 | −1.976 | 59.275 | 1.00 | 16.36 |
| ATOM | 2331 | CA | GLU | 331 | 30.149 | −1.105 | 58.998 | 1.00 | 18.14 |
| ATOM | 2332 | CB | GLU | 331 | 28.865 | −1.889 | 59.308 | 1.00 | 22.08 |
| ATOM | 2333 | CG | GLU | 331 | 28.790 | −3.226 | 58.546 | 1.00 | 26.82 |
| ATOM | 2334 | CD | GLU | 331 | 28.183 | −4.382 | 59.346 | 1.00 | 28.86 |
| ATOM | 2335 | OE1 | GLU | 331 | 28.381 | −5.552 | 58.931 | 1.00 | 28.12 |
| ATOM | 2336 | OE2 | GLU | 331 | 27.509 | −4.129 | 60.371 | 1.00 | 30.16 |
| ATOM | 2337 | C | GLU | 331 | 30.126 | 0.248 | 59.719 | 1.00 | 16.36 |
| ATOM | 2338 | O | GLU | 331 | 30.596 | 0.380 | 60.849 | 1.00 | 16.97 |
| ATOM | 2339 | N | THR | 332 | 29.583 | 1.263 | 59.060 | 1.00 | 14.04 |
| ATOM | 2340 | CA | THR | 332 | 29.494 | 2.568 | 59.695 | 1.00 | 14.47 |
| ATOM | 2341 | CB | THR | 332 | 28.747 | 3.562 | 58.825 | 1.00 | 10.93 |
| ATOM | 2342 | OG1 | THR | 332 | 29.473 | 3.751 | 57.611 | 1.00 | 6.57 |
| ATOM | 2343 | CG2 | THR | 332 | 28.597 | 4.890 | 59.550 | 1.00 | 6.34 |
| ATOM | 2344 | C | THR | 332 | 28.725 | 2.382 | 60.994 | 1.00 | 18.42 |
| ATOM | 2345 | O | THR | 332 | 29.125 | 2.872 | 62.052 | 1.00 | 17.70 |
| ATOM | 2346 | N | ARG | 333 | 27.609 | 1.671 | 60.892 | 1.00 | 21.79 |
| ATOM | 2347 | CA | ARG | 333 | 26.783 | 1.346 | 62.040 | 1.00 | 24.44 |
| ATOM | 2348 | CB | ARG | 333 | 26.095 | 0.001 | 61.764 | 1.00 | 28.62 |
| ATOM | 2349 | CG | ARG | 333 | 25.291 | −0.590 | 62.910 | 1.00 | 34.65 |
| ATOM | 2350 | CD | ARG | 333 | 24.308 | −1.664 | 62.401 | 1.00 | 39.87 |
| ATOM | 2351 | NE | ARG | 333 | 24.953 | −2.887 | 61.910 | 1.00 | 43.42 |
| ATOM | 2352 | CZ | ARG | 333 | 25.198 | −3.969 | 62.653 | 1.00 | 46.01 |
| ATOM | 2353 | NH1 | ARG | 333 | 24.852 | −3.992 | 63.940 | 1.00 | 45.10 |
| ATOM | 2354 | NH2 | ARG | 333 | 25.791 | −5.030 | 62.104 | 1.00 | 43.75 |
| ATOM | 2355 | C | ARG | 333 | 27.638 | 1.271 | 63.323 | 1.00 | 24.88 |
| ATOM | 2356 | O | ARG | 333 | 27.242 | 1.803 | 64.358 | 1.00 | 24.00 |
| ATOM | 2357 | N | PHE | 334 | 28.818 | 0.635 | 63.232 | 1.00 | 23.97 |
| ATOM | 2358 | CA | PHE | 334 | 29.740 | 0.458 | 64.371 | 1.00 | 19.64 |
| ATOM | 2359 | CB | PHE | 334 | 30.877 | −0.509 | 64.033 | 1.00 | 20.52 |
| ATOM | 2360 | CG | PHE | 334 | 30.420 | −1.813 | 63.468 | 1.00 | 24.74 |
| ATOM | 2361 | CD1 | PHE | 334 | 29.469 | −2.574 | 64.121 | 1.00 | 25.94 |
| ATOM | 2362 | CD2 | PHE | 334 | 30.938 | −2.279 | 62.262 | 1.00 | 26.47 |
| ATOM | 2363 | CE1 | PHE | 334 | 29.039 | −3.780 | 63.575 | 1.00 | 28.43 |
| ATOM | 2364 | CE2 | PHE | 334 | 30.514 | −3.483 | 61.711 | 1.00 | 24.74 |
| ATOM | 2365 | CZ | PHE | 334 | 29.565 | −4.233 | 62.365 | 1.00 | 26.41 |
| ATOM | 2366 | C | PHE | 334 | 30.382 | 1.739 | 64.842 | 1.00 | 16.52 |
| ATOM | 2367 | O | PHE | 334 | 30.434 | 2.020 | 66.039 | 1.00 | 16.16 |
| ATOM | 2368 | N | VAL | 335 | 30.907 | 2.509 | 63.905 | 1.00 | 13.20 |
| ATOM | 2369 | CA | VAL | 335 | 31.546 | 3.752 | 64.284 | 1.00 | 11.36 |
| ATOM | 2370 | CB | VAL | 335 | 31.877 | 4.565 | 63.033 | 1.00 | 8.08 |
| ATOM | 2371 | CG1 | VAL | 335 | 32.113 | 6.003 | 63.402 | 1.00 | 8.71 |
| ATOM | 2372 | CG2 | VAL | 335 | 33.082 | 3.979 | 62.358 | 1.00 | 1.00 |
| ATOM | 2373 | C | VAL | 335 | 30.653 | 4.558 | 65.249 | 1.00 | 13.02 |
| ATOM | 2374 | O | VAL | 335 | 31.126 | 5.066 | 66.264 | 1.00 | 10.40 |
| ATOM | 2375 | N | SER | 336 | 29.359 | 4.640 | 64.934 | 1.00 | 16.23 |
| ATOM | 2376 | CA | SER | 336 | 28.365 | 5.372 | 65.740 | 1.00 | 18.55 |
| ATOM | 2377 | CB | SER | 336 | 27.017 | 5.350 | 65.039 | 1.00 | 19.92 |
| ATOM | 2378 | OG | SER | 336 | 26.611 | 3.999 | 64.866 | 1.00 | 25.40 |
| ATOM | 2379 | C | SER | 336 | 28.162 | 4.766 | 67.118 | 1.00 | 17.99 |
| ATOM | 2380 | O | SER | 336 | 27.896 | 5.465 | 68.100 | 1.00 | 14.64 |
| ATOM | 2381 | N | GLN | 337 | 28.239 | 3.445 | 67.159 | 1.00 | 19.48 |
| ATOM | 2382 | CA | GLN | 337 | 28.061 | 2.719 | 68.394 | 1.00 | 21.39 |
| ATOM | 2383 | CB | GLN | 337 | 27.995 | 1.223 | 68.123 | 1.00 | 21.42 |
| ATOM | 2384 | CG | GLN | 337 | 26.829 | 0.800 | 67.264 | 1.00 | 23.07 |
| ATOM | 2385 | CD | GLN | 337 | 26.920 | −0.654 | 66.895 | 1.00 | 24.96 |
| ATOM | 2386 | OE1 | GLN | 337 | 27.243 | −1.496 | 67.735 | 1.00 | 28.83 |
| ATOM | 2387 | NE2 | GLN | 337 | 26.633 | −0.966 | 65.638 | 1.00 | 24.29 |
| ATOM | 2388 | C | GLN | 337 | 29.260 | 3.011 | 69.240 | 1.00 | 20.91 |
| ATOM | 2389 | O | GLN | 337 | 29.205 | 2.963 | 70.464 | 1.00 | 23.32 |
| ATOM | 2390 | N | VAL | 338 | 30.362 | 3.317 | 68.584 | 1.00 | 20.52 |
| ATOM | 2391 | CA | VAL | 338 | 31.559 | 3.589 | 69.337 | 1.00 | 21.67 |
| ATOM | 2392 | CB | VAL | 338 | 32.812 | 3.470 | 68.443 | 1.00 | 20.93 |
| ATOM | 2393 | CG1 | VAL | 338 | 34.065 | 3.624 | 69.279 | 1.00 | 19.79 |
| ATOM | 2394 | CG2 | VAL | 338 | 32.811 | 2.126 | 67.739 | 1.00 | 16.69 |
| ATOM | 2395 | C | VAL | 338 | 31.480 | 4.973 | 69.977 | 1.00 | 23.61 |
| ATOM | 2396 | O | VAL | 338 | 31.385 | 5.079 | 71.203 | 1.00 | 21.96 |
| ATOM | 2397 | N | GLU | 339 | 31.486 | 6.020 | 69.146 | 1.00 | 25.05 |
| ATOM | 2398 | CA | GLU | 339 | 31.455 | 7.406 | 69.620 | 1.00 | 26.21 |
| ATOM | 2399 | CB | GLU | 339 | 31.460 | 8.402 | 68.440 | 1.00 | 26.37 |
| ATOM | 2400 | CG | GLU | 339 | 30.515 | 8.082 | 67.282 | 1.00 | 31.63 |
| ATOM | 2401 | CD | GLU | 339 | 30.287 | 9.267 | 66.311 | 1.00 | 36.86 |
| ATOM | 2402 | OE1 | GLU | 339 | 29.542 | 10.219 | 66.663 | 1.00 | 37.19 |
| ATOM | 2403 | OE2 | GLU | 339 | 30.850 | 9.243 | 65.187 | 1.00 | 37.90 |
| ATOM | 2404 | C | GLU | 339 | 30.299 | 7.735 | 70.541 | 1.00 | 26.44 |
| ATOM | 2405 | O | GLU | 339 | 30.423 | 8.613 | 71.396 | 1.00 | 27.55 |
| ATOM | 2406 | N | SER | 340 | 29.189 | 7.017 | 70.380 | 1.00 | 26.30 |
| ATOM | 2407 | CA | SER | 340 | 27.987 | 7.246 | 71.181 | 1.00 | 25.08 |
| ATOM | 2408 | CB | SER | 340 | 26.861 | 6.322 | 70.717 | 1.00 | 23.68 |
| ATOM | 2409 | OG | SER | 340 | 27.191 | 4.970 | 70.957 | 1.00 | 23.58 |
| ATOM | 2410 | C | SER | 340 | 28.211 | 7.065 | 72.676 | 1.00 | 26.02 |
| ATOM | 2411 | O | SER | 340 | 27.415 | 7.539 | 73.488 | 1.00 | 26.83 |
| ATOM | 2412 | N | ASP | 341 | 29.294 | 6.380 | 73.033 | 1.00 | 27.41 |
| ATOM | 2413 | CA | ASP | 341 | 29.630 | 6.143 | 74.434 | 1.00 | 27.85 |
| ATOM | 2414 | CB | ASP | 341 | 28.939 | 4.885 | 74.953 | 1.00 | 27.41 |
| ATOM | 2415 | CG | ASP | 341 | 29.253 | 4.621 | 76.410 | 1.00 | 26.49 |
| ATOM | 2416 | OD1 | ASP | 341 | 29.628 | 5.591 | 77.107 | 1.00 | 26.07 |
| ATOM | 2417 | OD2 | ASP | 341 | 29.117 | 3.463 | 76.862 | 1.00 | 25.64 |
| ATOM | 2418 | C | ASP | 341 | 31.128 | 6.008 | 74.672 | 1.00 | 28.59 |
| ATOM | 2419 | O | ASP | 341 | 31.757 | 5.049 | 74.229 | 1.00 | 30.06 |
| ATOM | 2420 | N | THR | 342 | 31.688 | 6.965 | 75.398 | 1.00 | 27.34 |
| ATOM | 2421 | CA | THR | 342 | 33.105 | 6.953 | 75.694 | 1.00 | 26.74 |
| ATOM | 2422 | CB | THR | 342 | 33.681 | 8.348 | 75.553 | 1.00 | 26.75 |
| ATOM | 2423 | OG1 | THR | 342 | 33.072 | 9.217 | 76.511 | 1.00 | 25.10 |
| ATOM | 2424 | CG2 | THR | 342 | 33.387 | 8.881 | 74.171 | 1.00 | 29.29 |
| ATOM | 2425 | C | THR | 342 | 33.292 | 6.477 | 77.114 | 1.00 | 27.84 |
| ATOM | 2426 | O | THR | 342 | 34.365 | 6.625 | 77.692 | 1.00 | 27.29 |
| ATOM | 2427 | N | GLY | 343 | 32.223 | 5.908 | 77.662 | 1.00 | 30.32 |
| ATOM | 2428 | CA | GLY | 343 | 32.234 | 5.398 | 79.020 | 1.00 | 31.31 |
| ATOM | 2429 | C | GLY | 343 | 32.970 | 4.083 | 79.178 | 1.00 | 32.13 |
| ATOM | 2430 | O | GLY | 343 | 33.765 | 3.944 | 80.105 | 1.00 | 34.00 |
| ATOM | 2431 | N | ASP | 344 | 32.712 | 3.114 | 78.304 | 1.00 | 31.93 |
| ATOM | 2432 | CA | ASP | 344 | 33.400 | 1.836 | 78.411 | 1.00 | 34.25 |
| ATOM | 2433 | CB | ASP | 344 | 32.592 | 0.857 | 79.267 | 1.00 | 38.13 |
| ATOM | 2434 | CG | ASP | 344 | 31.205 | 0.646 | 78.744 | 1.00 | 43.49 |
| ATOM | 2435 | OD1 | ASP | 344 | 30.399 | −0.029 | 79.426 | 1.00 | 47.59 |
| ATOM | 2436 | OD2 | ASP | 344 | 30.923 | 1.159 | 77.643 | 1.00 | 46.67 |
| ATOM | 2437 | C | ASP | 344 | 33.744 | 1.196 | 77.075 | 1.00 | 33.85 |
| ATOM | 2438 | O | ASP | 344 | 33.354 | 1.681 | 76.015 | 1.00 | 32.12 |
| ATOM | 2439 | N | ARG | 345 | 34.490 | 0.098 | 77.148 | 1.00 | 34.54 |
| ATOM | 2440 | CA | ARG | 345 | 34.935 | −0.626 | 75.968 | 1.00 | 35.60 |
| ATOM | 2441 | CB | ARG | 345 | 36.297 | −1.278 | 76.233 | 1.00 | 35.33 |
| ATOM | 2442 | CG | ARG | 345 | 37.339 | −0.370 | 76.864 | 1.00 | 35.88 |
| ATOM | 2443 | CD | ARG | 345 | 38.729 | −1.006 | 76.879 | 1.00 | 35.19 |
| ATOM | 2444 | NE | ARG | 345 | 39.507 | −0.597 | 78.054 | 1.00 | 36.95 |
| ATOM | 2445 | CZ | ARG | 345 | 39.984 | 0.629 | 78.275 | 1.00 | 36.97 |
| ATOM | 2446 | NH1 | ARG | 345 | 39.780 | 1.605 | 77.396 | 1.00 | 36.40 |
| ATOM | 2447 | NH2 | ARG | 345 | 40.654 | 0.885 | 79.394 | 1.00 | 36.46 |
| ATOM | 2448 | C | ARG | 345 | 33.961 | −1.716 | 75.551 | 1.00 | 36.31 |
| ATOM | 2449 | O | ARG | 345 | 34.080 | −2.280 | 74.461 | 1.00 | 37.64 |
| ATOM | 2450 | N | LYS | 346 | 33.004 | −2.020 | 76.420 | 1.00 | 35.01 |
| ATOM | 2451 | CA | LYS | 346 | 32.050 | −3.081 | 76.134 | 1.00 | 33.81 |
| ATOM | 2452 | CB | LYS | 346 | 30.824 | −2.975 | 77.041 | 1.00 | 33.64 |
| ATOM | 2453 | CG | LYS | 346 | 29.942 | −4.223 | 76.985 | 1.00 | 33.85 |
| ATOM | 2454 | CD | LYS | 346 | 30.759 | −5.505 | 77.186 | 1.00 | 31.48 |
| ATOM | 2455 | CE | LYS | 346 | 30.061 | −6.699 | 76.542 | 1.00 | 32.39 |
| ATOM | 2456 | NZ | LYS | 346 | 30.855 | −7.968 | 76.542 | 1.00 | 30.01 |
| ATOM | 2457 | C | LYS | 346 | 31.613 | −3.093 | 74.684 | 1.00 | 33.18 |
| ATOM | 2458 | O | LYS | 346 | 31.746 | −4.101 | 73.995 | 1.00 | 31.98 |
| ATOM | 2459 | N | GLN | 347 | 31.101 | −1.967 | 74.214 | 1.00 | 33.36 |
| ATOM | 2460 | CA | GLN | 347 | 30.662 | −1.887 | 72.839 | 1.00 | 34.32 |
| ATOM | 2461 | CB | GLN | 347 | 30.014 | −0.530 | 72.589 | 1.00 | 37.17 |
| ATOM | 2462 | CG | GLN | 347 | 28.510 | −0.578 | 72.703 | 1.00 | 39.97 |
| ATOM | 2463 | CD | GLN | 347 | 27.905 | −1.436 | 71.611 | 1.00 | 43.97 |
| ATOM | 2464 | OE1 | GLN | 347 | 28.219 | −2.626 | 71.491 | 1.00 | 43.88 |
| ATOM | 2465 | NE2 | GLN | 347 | 27.039 | −0.835 | 70.799 | 1.00 | 46.46 |
| ATOM | 2466 | C | GLN | 347 | 31.799 | −2.144 | 71.844 | 1.00 | 34.27 |
| ATOM | 2467 | O | GLN | 347 | 31.630 | −2.922 | 70.902 | 1.00 | 35.29 |
| ATOM | 2468 | N | ILE | 348 | 32.952 | −1.502 | 72.054 | 1.00 | 31.49 |
| ATOM | 2469 | CA | ILE | 348 | 34.109 | −1.679 | 71.165 | 1.00 | 25.43 |
| ATOM | 2470 | CB | ILE | 348 | 35.309 | −0.826 | 71.614 | 1.00 | 21.01 |
| ATOM | 2471 | CG2 | ILE | 348 | 36.369 | −0.826 | 70.540 | 1.00 | 15.50 |
| ATOM | 2472 | CG1 | ILE | 348 | 34.852 | 0.606 | 71.875 | 1.00 | 22.27 |
| ATOM | 2473 | CD1 | ILE | 348 | 35.914 | 1.509 | 72.462 | 1.00 | 24.55 |
| ATOM | 2474 | C | ILE | 348 | 34.524 | −3.139 | 71.211 | 1.00 | 24.70 |
| ATOM | 2475 | O | ILE | 348 | 34.793 | −3.763 | 70.182 | 1.00 | 23.36 |
| ATOM | 2476 | N | TYR | 349 | 34.560 | −3.681 | 72.421 | 1.00 | 23.30 |
| ATOM | 2477 | CA | TYR | 349 | 34.933 | −5.061 | 72.597 | 1.00 | 23.65 |
| ATOM | 2478 | CB | TYR | 349 | 34.727 | −5.491 | 74.047 | 1.00 | 25.21 |
| ATOM | 2479 | CG | TYR | 349 | 34.779 | −6.989 | 74.221 | 1.00 | 31.27 |
| ATOM | 2480 | CD1 | TYR | 349 | 35.990 | −7.665 | 74.333 | 1.00 | 33.98 |
| ATOM | 2481 | CE1 | TYR | 349 | 36.028 | −9.062 | 74.435 | 1.00 | 36.98 |
| ATOM | 2482 | CD2 | TYR | 349 | 33.607 | −7.740 | 74.216 | 1.00 | 34.38 |
| ATOM | 2483 | CE2 | TYR | 349 | 33.628 | −9.125 | 74.312 | 1.00 | 36.69 |
| ATOM | 2484 | CZ | TYR | 349 | 34.837 | −9.786 | 74.421 | 1.00 | 37.89 |
| ATOM | 2485 | OH | TYR | 349 | 34.834 | −11.165 | 74.512 | 1.00 | 37.12 |
| ATOM | 2486 | C | TYR | 349 | 34.105 | −5.945 | 71.676 | 1.00 | 23.47 |
| ATOM | 2487 | O | TYR | 349 | 34.654 | −6.602 | 70.794 | 1.00 | 21.02 |
| ATOM | 2488 | N | ASN | 350 | 32.783 | −5.934 | 71.872 | 1.00 | 25.29 |
| ATOM | 2489 | CA | ASN | 350 | 31.850 | −6.766 | 71.091 | 1.00 | 25.07 |
| ATOM | 2490 | CB | ASN | 350 | 30.379 | −6.500 | 71.482 | 1.00 | 23.90 |
| ATOM | 2491 | CG | ASN | 350 | 30.069 | −6.844 | 72.941 | 1.00 | 25.09 |
| ATOM | 2492 | OD1 | ASN | 350 | 30.413 | −7.924 | 73.440 | 1.00 | 22.84 |
| ATOM | 2493 | ND2 | ASN | 350 | 29.398 | −5.923 | 73.626 | 1.00 | 25.65 |
| ATOM | 2494 | C | ASN | 350 | 31.982 | −6.620 | 69.580 | 1.00 | 25.25 |
| ATOM | 2495 | O | ASN | 350 | 31.994 | −7.619 | 68.859 | 1.00 | 25.84 |
| ATOM | 2496 | N | ILE | 351 | 32.068 | −5.392 | 69.083 | 1.00 | 25.43 |
| ATOM | 2497 | CA | ILE | 351 | 32.195 | −5.227 | 67.642 | 1.00 | 25.64 |
| ATOM | 2498 | CB | ILE | 351 | 32.388 | −3.745 | 67.248 | 1.00 | 24.60 |
| ATOM | 2499 | CG2 | ILE | 351 | 32.282 | −3.600 | 65.743 | 1.00 | 23.69 |
| ATOM | 2500 | CG1 | ILE | 351 | 31.305 | −2.882 | 67.903 | 1.00 | 22.24 |
| ATOM | 2501 | CD1 | ILE | 351 | 31.357 | −1.431 | 67.509 | 1.00 | 19.88 |
| ATOM | 2502 | C | ILE | 351 | 33.415 | −6.047 | 67.224 | 1.00 | 26.73 |
| ATOM | 2503 | O | ILE | 351 | 33.282 | −7.047 | 66.517 | 1.00 | 25.71 |
| ATOM | 2504 | N | LEU | 352 | 34.592 | −5.629 | 67.695 | 1.00 | 27.08 |
| ATOM | 2505 | CA | LEU | 352 | 35.847 | −6.312 | 67.397 | 1.00 | 27.36 |
| ATOM | 2506 | CB | LEU | 352 | 36.994 | −5.700 | 68.206 | 1.00 | 24.45 |
| ATOM | 2507 | CG | LEU | 352 | 37.295 | −4.208 | 68.090 | 1.00 | 23.84 |
| ATOM | 2508 | CD1 | LEU | 352 | 38.464 | −3.838 | 68.995 | 1.00 | 21.54 |
| ATOM | 2509 | CD2 | LEU | 352 | 37.620 | −3.872 | 66.660 | 1.00 | 23.96 |
| ATOM | 2510 | C | LEU | 352 | 35.746 | −7.798 | 67.737 | 1.00 | 29.42 |
| ATOM | 2511 | O | LEU | 352 | 36.045 | −8.670 | 66.912 | 1.00 | 29.43 |
| ATOM | 2512 | N | SER | 353 | 35.336 | −8.087 | 68.965 | 1.00 | 30.73 |
| ATOM | 2513 | CA | SER | 353 | 35.206 | −9.468 | 69.398 | 1.00 | 32.72 |
| ATOM | 2514 | CB | SER | 353 | 34.408 | −9.531 | 70.711 | 1.00 | 32.86 |
| ATOM | 2515 | OG | SER | 353 | 34.187 | −10.870 | 71.126 | 1.00 | 35.10 |
| ATOM | 2516 | C | SER | 353 | 34.513 | −10.277 | 68.295 | 1.00 | 33.76 |
| ATOM | 2517 | O | SER | 353 | 35.123 | −11.149 | 67.670 | 1.00 | 34.42 |
| ATOM | 2518 | N | THR | 354 | 33.252 | −9.941 | 68.035 | 1.00 | 34.17 |
| ATOM | 2519 | CA | THR | 354 | 32.437 | −10.621 | 67.031 | 1.00 | 32.96 |
| ATOM | 2520 | CB | THR | 354 | 30.999 | −10.073 | 67.076 | 1.00 | 33.01 |
| ATOM | 2521 | OG1 | THR | 354 | 30.120 | −10.980 | 66.408 | 1.00 | 32.52 |
| ATOM | 2522 | CG2 | THR | 354 | 30.922 | −8.702 | 66.411 | 1.00 | 34.65 |
| ATOM | 2523 | C | THR | 354 | 33.007 | −10.503 | 65.608 | 1.00 | 32.28 |
| ATOM | 2524 | O | THR | 354 | 32.444 | −11.038 | 64.646 | 1.00 | 30.58 |
| ATOM | 2525 | N | LEU | 355 | 34.137 | −9.807 | 65.497 | 1.00 | 31.47 |
| ATOM | 2526 | CA | LEU | 355 | 34.832 | −9.612 | 64.227 | 1.00 | 30.67 |
| ATOM | 2527 | CB | LEU | 355 | 35.488 | −8.239 | 64.187 | 1.00 | 28.42 |
| ATOM | 2528 | CG | LEU | 355 | 34.780 | −7.240 | 63.293 | 1.00 | 27.13 |
| ATOM | 2529 | CD1 | LEU | 355 | 35.387 | −5.874 | 63.487 | 1.00 | 26.09 |
| ATOM | 2530 | CD2 | LEU | 355 | 34.898 | −7.698 | 61.859 | 1.00 | 27.39 |
| ATOM | 2531 | C | LEU | 355 | 35.905 | −10.668 | 64.061 | 1.00 | 31.14 |
| ATOM | 2532 | O | LEU | 355 | 36.573 | −10.735 | 63.033 | 1.00 | 30.59 |
| ATOM | 2533 | N | GLY | 356 | 36.074 | −11.484 | 65.091 | 1.00 | 32.64 |
| ATOM | 2534 | CA | GLY | 356 | 37.068 | −12.530 | 65.030 | 1.00 | 35.49 |
| ATOM | 2535 | C | GLY | 356 | 38.435 | −12.074 | 65.493 | 1.00 | 37.44 |
| ATOM | 2536 | O | GLY | 356 | 39.443 | −12.492 | 64.930 | 1.00 | 37.31 |
| ATOM | 2537 | N | LEU | 357 | 38.471 | −11.222 | 66.516 | 1.00 | 39.40 |
| ATOM | 2538 | CA | LEU | 357 | 39.729 | −10.717 | 67.057 | 1.00 | 41.85 |
| ATOM | 2539 | CB | LEU | 357 | 39.898 | −9.239 | 66.705 | 1.00 | 41.35 |
| ATOM | 2540 | CG | LEU | 357 | 39.816 | −8.876 | 65.218 | 1.00 | 43.17 |
| ATOM | 2541 | CD1 | LEU | 357 | 39.953 | −7.375 | 65.064 | 1.00 | 42.98 |
| ATOM | 2542 | CD2 | LEU | 357 | 40.904 | −9.585 | 64.428 | 1.00 | 43.93 |
| ATOM | 2543 | C | LEU | 357 | 39.759 | −10.888 | 68.571 | 1.00 | 44.59 |
| ATOM | 2544 | O | LEU | 357 | 38.750 | −11.247 | 69.176 | 1.00 | 45.94 |
| ATOM | 2545 | N | ARG | 358 | 40.919 | −10.643 | 69.178 | 1.00 | 46.55 |
| ATOM | 2546 | CA | ARG | 358 | 41.080 | −10.752 | 70.632 | 1.00 | 48.12 |
| ATOM | 2547 | CB | ARG | 358 | 42.113 | −11.819 | 70.994 | 1.00 | 52.19 |
| ATOM | 2548 | CG | ARG | 358 | 41.649 | −13.258 | 70.839 | 1.00 | 61.21 |
| ATOM | 2549 | CD | ARG | 358 | 40.870 | −13.768 | 72.064 | 1.00 | 68.48 |
| ATOM | 2550 | NE | ARG | 358 | 39.519 | −13.206 | 72.184 | 1.00 | 74.00 |
| ATOM | 2551 | CZ | ARG | 358 | 38.629 | −13.577 | 73.104 | 1.00 | 75.57 |
| ATOM | 2552 | NH1 | ARG | 358 | 38.935 | −14.517 | 73.998 | 1.00 | 75.58 |
| ATOM | 2553 | NH2 | ARG | 358 | 37.431 | −13.005 | 73.131 | 1.00 | 74.54 |
| ATOM | 2554 | C | ARG | 358 | 41.558 | −9.418 | 71.174 | 1.00 | 46.76 |
| ATOM | 2555 | O | ARG | 358 | 42.702 | −9.284 | 71.580 | 1.00 | 49.52 |
| ATOM | 2556 | N | PRO | 359 | 40.679 | −8.412 | 71.197 | 1.00 | 45.33 |
| ATOM | 2557 | CD | PRO | 359 | 39.271 | −8.532 | 70.791 | 1.00 | 45.90 |
| ATOM | 2558 | CA | PRO | 359 | 40.956 | −7.056 | 71.677 | 1.00 | 44.06 |
| ATOM | 2559 | CB | PRO | 359 | 39.565 | −6.449 | 71.784 | 1.00 | 45.14 |
| ATOM | 2560 | CG | PRO | 359 | 38.865 | −7.086 | 70.643 | 1.00 | 46.70 |
| ATOM | 2561 | C | PRO | 359 | 41.725 | −6.936 | 72.986 | 1.00 | 42.11 |
| ATOM | 2562 | O | PRO | 359 | 41.662 | −7.797 | 73.860 | 1.00 | 42.98 |
| ATOM | 2563 | N | SER | 360 | 42.449 | −5.840 | 73.118 | 1.00 | 38.55 |
| ATOM | 2564 | CA | SER | 360 | 43.209 | −5.608 | 74.321 | 1.00 | 35.42 |
| ATOM | 2565 | CB | SER | 360 | 44.701 | −5.624 | 74.014 | 1.00 | 38.45 |
| ATOM | 2566 | OG | SER | 360 | 45.100 | −4.379 | 73.453 | 1.00 | 37.32 |
| ATOM | 2567 | C | SER | 360 | 42.847 | −4.234 | 74.818 | 1.00 | 33.26 |
| ATOM | 2568 | O | SER | 360 | 42.530 | −3.345 | 74.028 | 1.00 | 30.55 |
| ATOM | 2569 | N | THR | 361 | 42.907 | −4.060 | 76.128 | 1.00 | 31.87 |
| ATOM | 2570 | CA | THR | 361 | 42.625 | −2.771 | 76.721 | 1.00 | 33.02 |
| ATOM | 2571 | CB | THR | 361 | 43.285 | −2.646 | 78.083 | 1.00 | 32.00 |
| ATOM | 2572 | OG1 | THR | 361 | 42.697 | −3.593 | 78.981 | 1.00 | 31.30 |
| ATOM | 2573 | CG2 | THR | 361 | 43.135 | −1.223 | 78.618 | 1.00 | 28.90 |
| ATOM | 2574 | C | THR | 361 | 43.162 | −1.637 | 75.853 | 1.00 | 35.59 |
| ATOM | 2575 | O | THR | 361 | 42.600 | −0.545 | 75.837 | 1.00 | 37.16 |
| ATOM | 2576 | N | THR | 362 | 44.253 | −1.879 | 75.135 | 1.00 | 37.62 |
| ATOM | 2577 | CA | THR | 362 | 44.812 | −0.819 | 74.303 | 1.00 | 37.63 |
| ATOM | 2578 | CB | THR | 362 | 46.341 | −0.949 | 74.156 | 1.00 | 38.04 |
| ATOM | 2579 | OG1 | THR | 362 | 46.950 | −0.981 | 75.453 | 1.00 | 37.77 |
| ATOM | 2580 | CG2 | THR | 362 | 46.890 | 0.242 | 73.395 | 1.00 | 37.49 |
| ATOM | 2581 | C | THR | 362 | 44.183 | −0.839 | 72.928 | 1.00 | 36.67 |
| ATOM | 2582 | O | THR | 362 | 43.758 | 0.194 | 72.416 | 1.00 | 34.48 |
| ATOM | 2583 | N | ASP | 363 | 44.132 | −2.032 | 72.345 | 1.00 | 37.88 |
| ATOM | 2584 | CA | ASP | 363 | 43.555 | −2.246 | 71.024 | 1.00 | 40.18 |
| ATOM | 2585 | CB | ASP | 363 | 43.238 | −3.729 | 70.842 | 1.00 | 42.13 |
| ATOM | 2586 | CG | ASP | 363 | 44.477 | −4.557 | 70.666 | 1.00 | 45.73 |
| ATOM | 2587 | OD1 | ASP | 363 | 44.433 | −5.779 | 70.932 | 1.00 | 49.54 |
| ATOM | 2588 | OD2 | ASP | 363 | 45.500 | −3.976 | 70.247 | 1.00 | 46.04 |
| ATOM | 2589 | C | ASP | 363 | 42.289 | −1.429 | 70.841 | 1.00 | 40.28 |
| ATOM | 2590 | O | ASP | 363 | 42.070 | −0.801 | 69.802 | 1.00 | 38.03 |
| ATOM | 2591 | N | CYS | 364 | 41.455 | −1.449 | 71.871 | 1.00 | 41.60 |
| ATOM | 2592 | CA | CYS | 364 | 40.197 | −0.724 | 71.849 | 1.00 | 41.33 |
| ATOM | 2593 | CB | CYS | 364 | 39.426 | −1.036 | 73.131 | 1.00 | 41.81 |
| ATOM | 2594 | SG | CYS | 364 | 39.078 | −2.818 | 73.225 | 1.00 | 41.98 |
| ATOM | 2595 | C | CYS | 364 | 40.447 | 0.766 | 71.685 | 1.00 | 39.78 |
| ATOM | 2596 | O | CYS | 364 | 39.991 | 1.382 | 70.721 | 1.00 | 37.44 |
| ATOM | 2597 | N | ASP | 365 | 41.194 | 1.333 | 72.622 | 1.00 | 38.65 |
| ATOM | 2598 | CA | ASP | 365 | 41.525 | 2.744 | 72.580 | 1.00 | 37.87 |
| ATOM | 2599 | CB | ASP | 365 | 42.498 | 3.060 | 73.709 | 1.00 | 39.53 |
| ATOM | 2600 | CG | ASP | 365 | 42.073 | 2.424 | 75.014 | 1.00 | 42.28 |
| ATOM | 2601 | OD1 | ASP | 365 | 40.887 | 2.000 | 75.096 | 1.00 | 43.06 |
| ATOM | 2602 | OD2 | ASP | 365 | 42.908 | 2.355 | 75.949 | 1.00 | 41.82 |
| ATOM | 2603 | C | ASP | 365 | 42.123 | 3.092 | 71.220 | 1.00 | 35.70 |
| ATOM | 2604 | O | ASP | 365 | 41.887 | 4.173 | 70.682 | 1.00 | 35.49 |
| ATOM | 2605 | N | ILE | 366 | 42.895 | 2.175 | 70.655 | 1.00 | 32.72 |
| ATOM | 2606 | CA | ILE | 366 | 43.469 | 2.428 | 69.347 | 1.00 | 31.21 |
| ATOM | 2607 | CB | ILE | 366 | 44.345 | 1.241 | 68.891 | 1.00 | 30.98 |
| ATOM | 2608 | CG2 | ILE | 366 | 44.878 | 1.488 | 67.482 | 1.00 | 30.08 |
| ATOM | 2609 | CG1 | ILE | 366 | 45.472 | 1.010 | 69.907 | 1.00 | 30.05 |
| ATOM | 2610 | CD1 | ILE | 366 | 46.426 | 2.165 | 70.071 | 1.00 | 26.19 |
| ATOM | 2611 | C | ILE | 366 | 42.292 | 2.622 | 68.384 | 1.00 | 30.65 |
| ATOM | 2612 | O | ILE | 366 | 42.140 | 3.686 | 67.790 | 1.00 | 29.65 |
| ATOM | 2613 | N | VAL | 367 | 41.451 | 1.598 | 68.255 | 1.00 | 29.81 |
| ATOM | 2614 | CA | VAL | 367 | 40.287 | 1.665 | 67.378 | 1.00 | 27.24 |
| ATOM | 2615 | CB | VAL | 367 | 39.397 | 0.424 | 67.541 | 1.00 | 26.77 |
| ATOM | 2616 | CG1 | VAL | 367 | 38.193 | 0.520 | 66.630 | 1.00 | 25.16 |
| ATOM | 2617 | CG2 | VAL | 367 | 40.190 | −0.817 | 67.220 | 1.00 | 27.90 |
| ATOM | 2618 | C | VAL | 367 | 39.453 | 2.910 | 67.657 | 1.00 | 26.82 |
| ATOM | 2619 | O | VAL | 367 | 39.061 | 3.606 | 66.727 | 1.00 | 27.16 |
| ATOM | 2620 | N | ARG | 368 | 39.171 | 3.191 | 68.927 | 1.00 | 25.49 |
| ATOM | 2621 | CA | ARG | 368 | 38.398 | 4.380 | 69.266 | 1.00 | 24.26 |
| ATOM | 2622 | CB | ARG | 368 | 38.431 | 4.644 | 70.772 | 1.00 | 23.73 |
| ATOM | 2623 | CG | ARG | 368 | 37.765 | 5.951 | 71.217 | 1.00 | 26.32 |
| ATOM | 2624 | CD | ARG | 368 | 36.239 | 5.948 | 71.033 | 1.00 | 32.00 |
| ATOM | 2625 | NE | ARG | 368 | 35.542 | 5.015 | 71.926 | 1.00 | 33.36 |
| ATOM | 2626 | CZ | ARG | 368 | 35.558 | 5.096 | 73.253 | 1.00 | 33.30 |
| ATOM | 2627 | NH1 | ARG | 368 | 36.237 | 6.069 | 73.843 | 1.00 | 36.87 |
| ATOM | 2628 | NH2 | ARG | 368 | 34.904 | 4.209 | 73.990 | 1.00 | 30.08 |
| ATOM | 2629 | C | ARG | 368 | 39.034 | 5.545 | 68.539 | 1.00 | 25.24 |
| ATOM | 2630 | O | ARG | 368 | 38.403 | 6.175 | 67.700 | 1.00 | 26.08 |
| ATOM | 2631 | N | ARG | 369 | 40.299 | 5.808 | 68.844 | 1.00 | 26.69 |
| ATOM | 2632 | CA | ARG | 369 | 41.022 | 6.905 | 68.226 | 1.00 | 28.80 |
| ATOM | 2633 | CB | ARG | 369 | 42.500 | 6.842 | 68.619 | 1.00 | 33.81 |
| ATOM | 2634 | CG | ARG | 369 | 42.992 | 8.041 | 69.421 | 1.00 | 41.54 |
| ATOM | 2635 | CD | ARG | 369 | 44.246 | 8.666 | 68.797 | 1.00 | 47.78 |
| ATOM | 2636 | NE | ARG | 369 | 44.827 | 9.709 | 69.642 | 1.00 | 53.83 |
| ATOM | 2637 | CZ | ARG | 369 | 45.436 | 9.479 | 70.803 | 1.00 | 57.34 |
| ATOM | 2638 | NH1 | ARG | 369 | 45.547 | 8.234 | 71.256 | 1.00 | 57.39 |
| ATOM | 2639 | NH2 | ARG | 369 | 45.925 | 10.492 | 71.517 | 1.00 | 58.51 |
| ATOM | 2640 | C | ARG | 369 | 40.888 | 6.941 | 66.704 | 1.00 | 27.66 |
| ATOM | 2641 | O | ARG | 369 | 40.898 | 8.017 | 66.116 | 1.00 | 27.35 |
| ATOM | 2642 | N | ALA | 370 | 40.760 | 5.778 | 66.071 | 1.00 | 28.23 |
| ATOM | 2643 | CA | ALA | 370 | 40.622 | 5.699 | 64.613 | 1.00 | 29.69 |
| ATOM | 2644 | CB | ALA | 370 | 40.779 | 4.264 | 64.144 | 1.00 | 27.18 |
| ATOM | 2645 | C | ALA | 370 | 39.266 | 6.218 | 64.184 | 1.00 | 32.49 |
| ATOM | 2646 | O | ALA | 370 | 39.155 | 7.084 | 63.313 | 1.00 | 33.37 |
| ATOM | 2647 | N | CYS | 371 | 38.229 | 5.663 | 64.797 | 1.00 | 35.80 |
| ATOM | 2648 | CA | CYS | 371 | 36.860 | 6.053 | 64.500 | 1.00 | 37.09 |
| ATOM | 2649 | CB | CYS | 371 | 35.892 | 5.310 | 65.427 | 1.00 | 37.67 |
| ATOM | 2650 | SG | CYS | 371 | 35.709 | 3.539 | 65.052 | 1.00 | 43.56 |
| ATOM | 2651 | C | CYS | 371 | 36.692 | 7.555 | 64.663 | 1.00 | 36.66 |
| ATOM | 2652 | O | CYS | 371 | 36.237 | 8.231 | 63.746 | 1.00 | 36.14 |
| ATOM | 2653 | N | GLU | 372 | 37.079 | 8.065 | 65.829 | 1.00 | 36.70 |
| ATOM | 2654 | CA | GLU | 372 | 36.962 | 9.482 | 66.140 | 1.00 | 37.83 |
| ATOM | 2655 | CB | GLU | 372 | 37.440 | 9.741 | 67.569 | 1.00 | 41.72 |
| ATOM | 2656 | CG | GLU | 372 | 37.405 | 11.202 | 67.993 | 1.00 | 50.44 |
| ATOM | 2657 | CD | GLU | 372 | 38.615 | 11.981 | 67.504 | 1.00 | 56.78 |
| ATOM | 2658 | OE1 | GLU | 372 | 39.747 | 11.656 | 67.940 | 1.00 | 60.05 |
| ATOM | 2659 | OE2 | GLU | 372 | 38.437 | 12.914 | 66.685 | 1.00 | 59.31 |
| ATOM | 2660 | C | GLU | 372 | 37.736 | 10.344 | 65.163 | 1.00 | 36.14 |
| ATOM | 2661 | O | GLU | 372 | 37.280 | 11.410 | 64.745 | 1.00 | 34.24 |
| ATOM | 2662 | N | SER | 373 | 38.917 | 9.890 | 64.793 | 1.00 | 37.31 |
| ATOM | 2663 | CA | SER | 373 | 39.703 | 10.662 | 63.856 | 1.00 | 39.48 |
| ATOM | 2664 | CB | SER | 373 | 41.095 | 10.040 | 63.694 | 1.00 | 40.54 |
| ATOM | 2665 | OG | SER | 373 | 41.014 | 8.697 | 63.253 | 1.00 | 41.31 |
| ATOM | 2666 | C | SER | 373 | 38.966 | 10.713 | 62.516 | 1.00 | 38.54 |
| ATOM | 2667 | O | SER | 373 | 38.778 | 11.790 | 61.953 | 1.00 | 39.30 |
| ATOM | 2668 | N | VAL | 374 | 38.528 | 9.552 | 62.029 | 1.00 | 35.74 |
| ATOM | 2669 | CA | VAL | 374 | 37.817 | 9.462 | 60.755 | 1.00 | 34.53 |
| ATOM | 2670 | CB | VAL | 374 | 37.519 | 7.987 | 60.388 | 1.00 | 33.30 |
| ATOM | 2671 | CG1 | VAL | 374 | 36.688 | 7.897 | 59.119 | 1.00 | 30.40 |
| ATOM | 2672 | CG2 | VAL | 374 | 38.811 | 7.257 | 60.186 | 1.00 | 34.78 |
| ATOM | 2673 | C | VAL | 374 | 36.512 | 10.250 | 60.736 | 1.00 | 35.17 |
| ATOM | 2674 | O | VAL | 374 | 36.253 | 11.010 | 59.797 | 1.00 | 34.51 |
| ATOM | 2675 | N | SER | 375 | 35.700 | 10.080 | 61.775 | 1.00 | 35.24 |
| ATOM | 2676 | CA | SER | 375 | 34.416 | 10.768 | 61.866 | 1.00 | 34.91 |
| ATOM | 2677 | CB | SER | 375 | 33.641 | 10.312 | 63.103 | 1.00 | 35.91 |
| ATOM | 2678 | OG | SER | 375 | 33.802 | 11.230 | 64.178 | 1.00 | 37.28 |
| ATOM | 2679 | C | SER | 375 | 34.585 | 12.272 | 61.933 | 1.00 | 34.67 |
| ATOM | 2680 | O | SER | 375 | 33.865 | 13.010 | 61.266 | 1.00 | 35.17 |
| ATOM | 2681 | N | THR | 376 | 35.534 | 12.725 | 62.743 | 1.00 | 34.00 |
| ATOM | 2682 | CA | THR | 376 | 35.786 | 14.150 | 62.889 | 1.00 | 35.55 |
| ATOM | 2683 | CB | THR | 376 | 36.827 | 14.421 | 63.954 | 1.00 | 38.06 |
| ATOM | 2684 | OG1 | THR | 376 | 36.461 | 13.739 | 65.158 | 1.00 | 40.51 |
| ATOM | 2685 | CG2 | THR | 376 | 36.926 | 15.923 | 64.239 | 1.00 | 38.22 |
| ATOM | 2686 | C | THR | 376 | 36.208 | 14.788 | 61.583 | 1.00 | 34.80 |
| ATOM | 2687 | O | THR | 376 | 35.794 | 15.901 | 61.241 | 1.00 | 32.23 |
| ATOM | 2688 | N | ARG | 377 | 37.049 | 14.078 | 60.848 | 1.00 | 36.51 |
| ATOM | 2689 | CA | ARG | 377 | 37.523 | 14.601 | 59.581 | 1.00 | 38.20 |
| ATOM | 2690 | CB | ARG | 377 | 38.535 | 13.640 | 58.956 | 1.00 | 41.90 |
| ATOM | 2691 | CG | ARG | 377 | 39.417 | 14.271 | 57.892 | 1.00 | 43.83 |
| ATOM | 2692 | CD | ARG | 377 | 38.735 | 14.280 | 56.551 | 1.00 | 46.24 |
| ATOM | 2693 | NE | ARG | 377 | 38.467 | 12.921 | 56.074 | 1.00 | 50.02 |
| ATOM | 2694 | CZ | ARG | 377 | 39.400 | 12.058 | 55.679 | 1.00 | 48.89 |
| ATOM | 2695 | NH1 | ARG | 377 | 40.681 | 12.405 | 55.700 | 1.00 | 47.77 |
| ATOM | 2696 | NH2 | ARG | 377 | 39.050 | 10.849 | 55.256 | 1.00 | 48.65 |
| ATOM | 2697 | C | ARG | 377 | 36.311 | 14.759 | 58.688 | 1.00 | 37.15 |
| ATOM | 2698 | O | ARG | 377 | 36.163 | 15.780 | 58.016 | 1.00 | 37.23 |
| ATOM | 2699 | N | ALA | 378 | 35.445 | 13.744 | 58.706 | 1.00 | 36.43 |
| ATOM | 2700 | CA | ALA | 378 | 34.212 | 13.732 | 57.920 | 1.00 | 35.58 |
| ATOM | 2701 | CB | ALA | 378 | 33.470 | 12.430 | 58.130 | 1.00 | 35.75 |
| ATOM | 2702 | C | ALA | 378 | 33.314 | 14.897 | 58.304 | 1.00 | 34.75 |
| ATOM | 2703 | O | ALA | 378 | 32.675 | 15.507 | 57.451 | 1.00 | 34.63 |
| ATOM | 2704 | N | ALA | 379 | 33.249 | 15.204 | 59.590 | 1.00 | 34.17 |
| ATOM | 2705 | CA | ALA | 379 | 32.427 | 16.317 | 60.009 | 1.00 | 34.54 |
| ATOM | 2706 | CB | ALA | 379 | 32.281 | 16.340 | 61.515 | 1.00 | 32.43 |
| ATOM | 2707 | C | ALA | 379 | 33.073 | 17.607 | 59.519 | 1.00 | 35.95 |
| ATOM | 2708 | O | ALA | 379 | 32.465 | 18.358 | 58.761 | 1.00 | 38.27 |
| ATOM | 2709 | N | HIS | 380 | 34.314 | 17.856 | 59.925 | 1.00 | 35.13 |
| ATOM | 2710 | CA | HIS | 380 | 34.994 | 19.083 | 59.526 | 1.00 | 34.04 |
| ATOM | 2711 | CB | HIS | 380 | 36.448 | 19.031 | 59.968 | 1.00 | 37.01 |
| ATOM | 2712 | CG | HIS | 380 | 36.628 | 19.284 | 61.430 | 1.00 | 42.02 |
| ATOM | 2713 | CD2 | HIS | 380 | 35.734 | 19.637 | 62.385 | 1.00 | 43.27 |
| ATOM | 2714 | ND1 | HIS | 380 | 37.852 | 19.206 | 62.058 | 1.00 | 44.66 |
| ATOM | 2715 | CE1 | HIS | 380 | 37.704 | 19.500 | 63.339 | 1.00 | 46.06 |
| ATOM | 2716 | NE2 | HIS | 380 | 36.429 | 19.766 | 63.562 | 1.00 | 44.63 |
| ATOM | 2717 | C | HIS | 380 | 34.894 | 19.405 | 58.045 | 1.00 | 32.37 |
| ATOM | 2718 | O | HIS | 380 | 34.581 | 20.536 | 57.671 | 1.00 | 29.98 |
| ATOM | 2719 | N | MET | 381 | 35.154 | 18.417 | 57.197 | 1.00 | 30.55 |
| ATOM | 2720 | CA | MET | 381 | 35.055 | 18.640 | 55.764 | 1.00 | 30.35 |
| ATOM | 2721 | CB | MET | 381 | 35.383 | 17.365 | 54.992 | 1.00 | 28.41 |
| ATOM | 2722 | CG | MET | 381 | 36.852 | 17.181 | 54.767 | 1.00 | 28.31 |
| ATOM | 2723 | SD | MET | 381 | 37.505 | 18.684 | 54.017 | 1.00 | 31.73 |
| ATOM | 2724 | CE | MET | 381 | 38.142 | 18.070 | 52.446 | 1.00 | 30.02 |
| ATOM | 2725 | C | MET | 381 | 33.647 | 19.101 | 55.415 | 1.00 | 32.29 |
| ATOM | 2726 | O | MET | 381 | 33.453 | 19.930 | 54.527 | 1.00 | 32.42 |
| ATOM | 2727 | N | CYS | 382 | 32.660 | 18.566 | 56.124 | 1.00 | 33.02 |
| ATOM | 2728 | CA | CYS | 382 | 31.279 | 18.942 | 55.869 | 1.00 | 33.44 |
| ATOM | 2729 | CB | CYS | 382 | 30.323 | 18.012 | 56.625 | 1.00 | 33.78 |
| ATOM | 2730 | SG | CYS | 382 | 28.582 | 18.152 | 56.124 | 1.00 | 40.21 |
| ATOM | 2731 | C | CYS | 382 | 31.087 | 20.387 | 56.316 | 1.00 | 33.02 |
| ATOM | 2732 | O | CYS | 382 | 30.566 | 21.218 | 55.563 | 1.00 | 32.71 |
| ATOM | 2733 | N | SER | 383 | 31.528 | 20.686 | 57.537 | 1.00 | 33.57 |
| ATOM | 2734 | CA | SER | 383 | 31.418 | 22.037 | 58.097 | 1.00 | 33.39 |
| ATOM | 2735 | CB | SER | 383 | 32.232 | 22.159 | 59.392 | 1.00 | 32.88 |
| ATOM | 2736 | OG | SER | 383 | 33.605 | 21.877 | 59.176 | 1.00 | 31.29 |
| ATOM | 2737 | C | SER | 383 | 31.935 | 23.042 | 57.085 | 1.00 | 32.50 |
| ATOM | 2738 | O | SER | 383 | 31.314 | 24.073 | 56.832 | 1.00 | 32.64 |
| ATOM | 2739 | N | ALA | 384 | 33.082 | 22.729 | 56.501 | 1.00 | 30.75 |
| ATOM | 2740 | CA | ALA | 384 | 33.663 | 23.607 | 55.510 | 1.00 | 29.62 |
| ATOM | 2741 | CB | ALA | 384 | 34.787 | 22.885 | 54.789 | 1.00 | 29.04 |
| ATOM | 2742 | C | ALA | 384 | 32.604 | 24.095 | 54.509 | 1.00 | 29.94 |
| ATOM | 2743 | O | ALA | 384 | 32.211 | 25.259 | 54.544 | 1.00 | 28.35 |
| ATOM | 2744 | N | GLY | 385 | 32.141 | 23.193 | 53.639 | 1.00 | 31.38 |
| ATOM | 2745 | CA | GLY | 385 | 31.149 | 23.525 | 52.621 | 1.00 | 30.00 |
| ATOM | 2746 | C | GLY | 385 | 29.870 | 24.198 | 53.090 | 1.00 | 30.54 |
| ATOM | 2747 | O | GLY | 385 | 29.522 | 25.285 | 52.613 | 1.00 | 28.88 |
| ATOM | 2748 | N | LEU | 386 | 29.151 | 23.559 | 54.010 | 1.00 | 29.58 |
| ATOM | 2749 | CA | LEU | 386 | 27.917 | 24.148 | 54.522 | 1.00 | 28.86 |
| ATOM | 2750 | CB | LEU | 386 | 27.410 | 23.374 | 55.749 | 1.00 | 25.55 |
| ATOM | 2751 | CG | LEU | 386 | 26.141 | 23.824 | 56.493 | 1.00 | 21.28 |
| ATOM | 2752 | CD1 | LEU | 386 | 26.504 | 24.768 | 57.605 | 1.00 | 18.56 |
| ATOM | 2753 | CD2 | LEU | 386 | 25.157 | 24.456 | 55.533 | 1.00 | 17.77 |
| ATOM | 2754 | C | LEU | 386 | 28.199 | 25.595 | 54.898 | 1.00 | 30.29 |
| ATOM | 2755 | O | LEU | 386 | 27.344 | 26.458 | 54.728 | 1.00 | 30.86 |
| ATOM | 2756 | N | ALA | 387 | 29.413 | 25.846 | 55.393 | 1.00 | 32.40 |
| ATOM | 2757 | CA | ALA | 387 | 29.851 | 27.184 | 55.799 | 1.00 | 32.84 |
| ATOM | 2758 | CB | ALA | 387 | 31.181 | 27.101 | 56.536 | 1.00 | 31.99 |
| ATOM | 2759 | C | ALA | 387 | 29.991 | 28.098 | 54.585 | 1.00 | 34.20 |
| ATOM | 2760 | O | ALA | 387 | 29.509 | 29.235 | 54.588 | 1.00 | 34.34 |
| ATOM | 2761 | N | GLY | 388 | 30.663 | 27.597 | 53.553 | 1.00 | 34.88 |
| ATOM | 2762 | CA | GLY | 388 | 30.831 | 28.378 | 52.344 | 1.00 | 35.13 |
| ATOM | 2763 | C | GLY | 388 | 29.467 | 28.833 | 51.867 | 1.00 | 35.42 |
| ATOM | 2764 | O | GLY | 388 | 29.257 | 30.005 | 51.545 | 1.00 | 36.39 |
| ATOM | 2765 | N | VAL | 389 | 28.524 | 27.898 | 51.839 | 1.00 | 34.42 |
| ATOM | 2766 | CA | VAL | 389 | 27.167 | 28.202 | 51.402 | 1.00 | 32.28 |
| ATOM | 2767 | CB | VAL | 389 | 26.266 | 26.949 | 51.487 | 1.00 | 31.56 |
| ATOM | 2768 | CG1 | VAL | 389 | 24.856 | 27.285 | 51.027 | 1.00 | 28.68 |
| ATOM | 2769 | CG2 | VAL | 389 | 26.853 | 25.836 | 50.638 | 1.00 | 28.05 |
| ATOM | 2770 | C | VAL | 389 | 26.579 | 29.307 | 52.273 | 1.00 | 30.86 |
| ATOM | 2771 | O | VAL | 389 | 26.072 | 30.304 | 51.762 | 1.00 | 26.91 |
| ATOM | 2772 | N | ILE | 390 | 26.665 | 29.115 | 53.586 | 1.00 | 31.06 |
| ATOM | 2773 | CA | ILE | 390 | 26.146 | 30.073 | 54.548 | 1.00 | 34.83 |
| ATOM | 2774 | CB | ILE | 390 | 26.262 | 29.538 | 56.001 | 1.00 | 32.76 |
| ATOM | 2775 | CG2 | ILE | 390 | 25.733 | 30.562 | 56.996 | 1.00 | 31.45 |
| ATOM | 2776 | CG1 | ILE | 390 | 25.425 | 28.274 | 56.154 | 1.00 | 32.06 |
| ATOM | 2777 | CD1 | ILE | 390 | 25.311 | 27.804 | 57.572 | 1.00 | 31.73 |
| ATOM | 2778 | C | ILE | 390 | 26.858 | 31.415 | 54.444 | 1.00 | 39.01 |
| ATOM | 2779 | O | ILE | 390 | 26.209 | 32.465 | 54.370 | 1.00 | 42.11 |
| ATOM | 2780 | N | ASN | 391 | 28.186 | 31.398 | 54.437 | 1.00 | 41.07 |
| ATOM | 2781 | CA | ASN | 391 | 28.921 | 32.652 | 54.326 | 1.00 | 42.97 |
| ATOM | 2782 | CB | ASN | 391 | 30.430 | 32.386 | 54.290 | 1.00 | 47.00 |
| ATOM | 2783 | CG | ASN | 391 | 31.061 | 32.452 | 55.678 | 1.00 | 51.32 |
| ATOM | 2784 | OD1 | ASN | 391 | 32.205 | 32.029 | 55.878 | 1.00 | 51.98 |
| ATOM | 2785 | ND2 | ASN | 391 | 30.312 | 32.996 | 56.646 | 1.00 | 51.87 |
| ATOM | 2786 | C | ASN | 391 | 28.459 | 33.377 | 53.070 | 1.00 | 42.59 |
| ATOM | 2787 | O | ASN | 391 | 27.927 | 34.488 | 53.141 | 1.00 | 40.64 |
| ATOM | 2788 | N | ARG | 392 | 28.638 | 32.723 | 51.928 | 1.00 | 43.30 |
| ATOM | 2789 | CA | ARG | 392 | 28.237 | 33.277 | 50.644 | 1.00 | 45.75 |
| ATOM | 2790 | CB | ARG | 392 | 28.328 | 32.182 | 49.571 | 1.00 | 48.20 |
| ATOM | 2791 | CG | ARG | 392 | 27.020 | 31.811 | 48.892 | 1.00 | 54.22 |
| ATOM | 2792 | CD | ARG | 392 | 26.803 | 32.578 | 47.590 | 1.00 | 59.36 |
| ATOM | 2793 | NE | ARG | 392 | 27.491 | 31.984 | 46.437 | 1.00 | 66.46 |
| ATOM | 2794 | CZ | ARG | 392 | 28.794 | 32.098 | 46.156 | 1.00 | 69.91 |
| ATOM | 2795 | NH1 | ARG | 392 | 29.613 | 32.793 | 46.941 | 1.00 | 69.61 |
| ATOM | 2796 | NH2 | ARG | 392 | 29.279 | 31.524 | 45.063 | 1.00 | 70.50 |
| ATOM | 2797 | C | ARG | 392 | 26.822 | 33.854 | 50.711 | 1.00 | 45.30 |
| ATOM | 2798 | O | ARG | 392 | 26.474 | 34.777 | 49.973 | 1.00 | 44.35 |
| ATOM | 2799 | N | MET | 393 | 26.009 | 33.316 | 51.607 | 1.00 | 47.21 |
| ATOM | 2800 | CA | MET | 393 | 24.640 | 33.785 | 51.739 | 1.00 | 50.87 |
| ATOM | 2801 | CB | MET | 393 | 23.761 | 32.687 | 52.346 | 1.00 | 49.84 |
| ATOM | 2802 | CG | MET | 393 | 23.427 | 31.551 | 51.389 | 1.00 | 45.97 |
| ATOM | 2803 | SD | MET | 393 | 22.244 | 30.416 | 52.096 | 1.00 | 42.67 |
| ATOM | 2804 | CE | MET | 393 | 20.761 | 31.465 | 52.244 | 1.00 | 42.67 |
| ATOM | 2805 | C | MET | 393 | 24.559 | 35.046 | 52.581 | 1.00 | 54.43 |
| ATOM | 2806 | O | MET | 393 | 23.631 | 35.851 | 52.443 | 1.00 | 53.85 |
| ATOM | 2807 | N | ARG | 394 | 25.528 | 35.208 | 53.469 | 1.00 | 59.09 |
| ATOM | 2808 | CA | ARG | 394 | 25.568 | 36.386 | 54.314 | 1.00 | 64.57 |
| ATOM | 2809 | CB | ARG | 394 | 26.624 | 36.224 | 55.404 | 1.00 | 65.91 |
| ATOM | 2810 | CG | ARG | 394 | 26.830 | 37.477 | 56.228 | 1.00 | 67.95 |
| ATOM | 2811 | CD | ARG | 394 | 28.048 | 37.364 | 57.130 | 1.00 | 69.02 |
| ATOM | 2812 | NE | ARG | 394 | 28.499 | 38.673 | 57.600 | 1.00 | 68.97 |
| ATOM | 2813 | CZ | ARG | 394 | 27.776 | 39.494 | 58.357 | 1.00 | 69.21 |
| ATOM | 2814 | NH1 | ARG | 394 | 26.553 | 39.151 | 58.743 | 1.00 | 70.35 |
| ATOM | 2815 | NH2 | ARG | 394 | 28.281 | 40.662 | 58.732 | 1.00 | 68.24 |
| ATOM | 2816 | C | ARG | 394 | 25.952 | 37.537 | 53.404 | 1.00 | 67.94 |
| ATOM | 2817 | O | ARG | 394 | 25.306 | 38.588 | 53.391 | 1.00 | 67.14 |
| ATOM | 2818 | N | GLU | 395 | 27.012 | 37.313 | 52.633 | 1.00 | 72.08 |
| ATOM | 2819 | CA | GLU | 395 | 27.513 | 38.314 | 51.707 | 1.00 | 77.04 |
| ATOM | 2820 | CB | GLU | 395 | 28.578 | 37.691 | 50.784 | 1.00 | 78.09 |
| ATOM | 2821 | CG | GLU | 395 | 29.425 | 38.685 | 49.955 | 1.00 | 81.99 |
| ATOM | 2822 | CD | GLU | 395 | 30.402 | 39.533 | 50.789 | 1.00 | 84.19 |
| ATOM | 2823 | OE1 | GLU | 395 | 29.949 | 10.442 | 51.526 | 1.00 | 83.64 |
| ATOM | 2824 | OE2 | GLU | 395 | 31.631 | 39.290 | 50.702 | 1.00 | 84.22 |
| ATOM | 2825 | C | GLU | 395 | 26.340 | 38.873 | 50.898 | 1.00 | 79.30 |
| ATOM | 2826 | O | GLU | 395 | 26.250 | 40.078 | 50.683 | 1.00 | 81.15 |
| ATOM | 2827 | N | SER | 396 | 25.423 | 38.007 | 50.481 | 1.00 | 81.59 |
| ATOM | 2828 | CA | SER | 396 | 24.276 | 38.451 | 49.696 | 1.00 | 83.40 |
| ATOM | 2829 | CB | SER | 396 | 23.379 | 37.264 | 49.366 | 1.00 | 84.05 |
| ATOM | 2830 | OG | SER | 396 | 24.123 | 36.252 | 48.716 | 1.00 | 86.28 |
| ATOM | 2831 | C | SER | 396 | 23.462 | 39.526 | 50.406 | 1.00 | 84.36 |
| ATOM | 2832 | O | SER | 396 | 23.578 | 40.708 | 50.092 | 1.00 | 84.49 |
| ATOM | 2833 | N | ARG | 397 | 22.639 | 39.118 | 51.362 | 1.00 | 86.41 |
| ATOM | 2834 | CA | ARG | 397 | 21.812 | 40.070 | 52.090 | 1.00 | 88.71 |
| ATOM | 2835 | CB | ARG | 397 | 20.682 | 39.335 | 52.816 | 1.00 | 89.74 |
| ATOM | 2836 | CG | ARG | 397 | 19.579 | 40.241 | 53.346 | 1.00 | 90.87 |
| ATOM | 2837 | CD | ARG | 397 | 19.096 | 39.776 | 54.713 | 1.00 | 91.04 |
| ATOM | 2838 | NE | ARG | 397 | 20.021 | 40.158 | 55.782 | 1.00 | 89.87 |
| ATOM | 2839 | CZ | ARG | 397 | 19.905 | 39.766 | 57.047 | 1.00 | 89.80 |
| ATOM | 2840 | NH1 | ARG | 397 | 18.906 | 38.971 | 57.409 | 1.00 | 91.15 |
| ATOM | 2841 | NH2 | ARG | 397 | 20.779 | 40.174 | 57.955 | 1.00 | 87.97 |
| ATOM | 2842 | C | ARG | 397 | 22.653 | 40.847 | 53.102 | 1.00 | 89.74 |
| ATOM | 2843 | O | ARG | 397 | 22.585 | 40.588 | 54.305 | 1.00 | 90.41 |
| ATOM | 2844 | N | SER | 398 | 23.448 | 41.795 | 52.614 | 1.00 | 90.58 |
| ATOM | 2845 | CA | SER | 398 | 24.288 | 42.602 | 53.492 | 1.00 | 91.09 |
| ATOM | 2846 | CB | SER | 398 | 24.903 | 43.782 | 52.718 | 1.00 | 91.14 |
| ATOM | 2847 | OG | SER | 398 | 25.845 | 43.347 | 51.747 | 1.00 | 89.49 |
| ATOM | 2848 | C | SER | 398 | 23.470 | 43.129 | 54.677 | 1.00 | 91.27 |
| ATOM | 2849 | O | SER | 398 | 22.458 | 43.810 | 54.496 | 1.00 | 91.10 |
| ATOM | 2850 | N | GLU | 399 | 23.904 | 42.786 | 55.887 | 1.00 | 91.43 |
| ATOM | 2851 | CA | GLU | 399 | 23.238 | 43.233 | 57.108 | 1.00 | 90.89 |
| ATOM | 2852 | CB | GLU | 399 | 21.799 | 42.705 | 57.183 | 1.00 | 91.87 |
| ATOM | 2853 | CG | GLU | 399 | 20.969 | 43.349 | 58.298 | 1.00 | 93.31 |
| ATOM | 2854 | CD | GLU | 399 | 20.726 | 44.836 | 58.064 | 1.00 | 94.22 |
| ATOM | 2855 | OE1 | GLU | 399 | 20.270 | 45.533 | 58.999 | 1.00 | 93.53 |
| ATOM | 2856 | OE2 | GLU | 399 | 20.986 | 45.307 | 56.936 | 1.00 | 94.80 |
| ATOM | 2857 | C | GLU | 399 | 24.013 | 42.774 | 58.339 | 1.00 | 89.25 |
| ATOM | 2858 | O | GLU | 399 | 24.987 | 42.029 | 58.236 | 1.00 | 88.96 |
| ATOM | 2859 | N | ASP | 400 | 23.570 | 43.226 | 59.502 | 1.00 | 87.38 |
| ATOM | 2860 | CA | ASP | 400 | 24.214 | 42.883 | 60.754 | 1.00 | 85.70 |
| ATOM | 2861 | CB | ASP | 400 | 23.332 | 43.352 | 61.915 | 1.00 | 88.35 |
| ATOM | 2862 | CG | ASP | 400 | 22.861 | 44.795 | 61.743 | 1.00 | 90.64 |
| ATOM | 2863 | OD1 | ASP | 400 | 22.059 | 45.055 | 60.817 | 1.00 | 91.42 |
| ATOM | 2864 | OD2 | ASP | 400 | 23.297 | 45.671 | 62.524 | 1.00 | 91.68 |
| ATOM | 2865 | C | ASP | 400 | 24.496 | 41.385 | 60.853 | 1.00 | 82.94 |
| ATOM | 2866 | O | ASP | 400 | 25.506 | 40.900 | 60.346 | 1.00 | 82.03 |
| ATOM | 2867 | N | VAL | 401 | 23.593 | 40.658 | 61.502 | 1.00 | 79.90 |
| ATOM | 2868 | CA | VAL | 401 | 23.738 | 39.219 | 61.682 | 1.00 | 75.91 |
| ATOM | 2869 | CB | VAL | 401 | 23.607 | 38.841 | 63.153 | 1.00 | 74.20 |
| ATOM | 2870 | CG1 | VAL | 401 | 24.803 | 39.343 | 63.927 | 1.00 | 73.12 |
| ATOM | 2871 | CG2 | VAL | 401 | 22.314 | 39.430 | 63.710 | 1.00 | 72.79 |
| ATOM | 2872 | C | VAL | 401 | 22.662 | 38.458 | 60.925 | 1.00 | 74.63 |
| ATOM | 2873 | O | VAL | 401 | 21.489 | 38.846 | 60.942 | 1.00 | 75.56 |
| ATOM | 2874 | N | MET | 402 | 23.063 | 37.365 | 60.278 | 1.00 | 70.61 |
| ATOM | 2875 | CA | MET | 402 | 22.130 | 36.539 | 59.521 | 1.00 | 65.65 |
| ATOM | 2876 | CB | MET | 402 | 22.818 | 35.887 | 58.325 | 1.00 | 62.74 |
| ATOM | 2877 | CG | MET | 402 | 21.897 | 34.958 | 57.543 | 1.00 | 56.61 |
| ATOM | 2878 | SD | MET | 402 | 22.543 | 34.551 | 55.906 | 1.00 | 52.49 |
| ATOM | 2879 | CE | MET | 402 | 23.857 | 33.399 | 56.323 | 1.00 | 49.76 |
| ATOM | 2880 | C | MET | 402 | 21.532 | 35.450 | 60.381 | 1.00 | 65.00 |
| ATOM | 2881 | 0 | MET | 402 | 22.222 | 34.513 | 60.781 | 1.00 | 65.18 |
| ATOM | 2882 | N | ARG | 403 | 20.241 | 35.575 | 60.657 | 1.00 | 63.62 |
| ATOM | 2883 | CA | ARG | 403 | 19.535 | 34.593 | 61.462 | 1.00 | 61.57 |
| ATOM | 2884 | CB | ARG | 403 | 18.418 | 35.275 | 62.262 | 1.00 | 64.83 |
| ATOM | 2885 | CG | ARG | 403 | 18.856 | 36.547 | 62.987 | 1.00 | 70.01 |
| ATOM | 2886 | CD | ARG | 403 | 17.691 | 37.205 | 63.724 | 1.00 | 75.36 |
| ATOM | 2887 | NE | ARG | 403 | 17.412 | 36.582 | 65.018 | 1.00 | 80.50 |
| ATOM | 2888 | CZ | ARG | 403 | 16.305 | 36.788 | 65.731 | 1.00 | 83.41 |
| ATOM | 2889 | NH1 | ARG | 403 | 15.358 | 37.603 | 65.277 | 1.00 | 84.55 |
| ATOM | 2890 | NH2 | ARG | 403 | 16.147 | 36.187 | 66.907 | 1.00 | 83.64 |
| ATOM | 2891 | C | ARG | 403 | 18.946 | 33.560 | 60.504 | 1.00 | 57.99 |
| ATOM | 2892 | O | ARG | 403 | 17.775 | 33.639 | 60.135 | 1.00 | 58.57 |
| ATOM | 2893 | N | ILE | 404 | 19.762 | 32.597 | 60.091 | 1.00 | 52.97 |
| ATOM | 2894 | CA | ILE | 404 | 19.301 | 31.570 | 59.170 | 1.00 | 49.14 |
| ATOM | 2895 | CB | ILE | 404 | 20.293 | 31.412 | 57.999 | 1.00 | 47.44 |
| ATOM | 2896 | CG2 | ILE | 404 | 21.538 | 30.680 | 58.458 | 1.00 | 43.94 |
| ATOM | 2897 | CG1 | ILE | 404 | 19.629 | 30.649 | 56.854 | 1.00 | 48.45 |
| ATOM | 2898 | CD1 | ILE | 404 | 20.477 | 30.559 | 55.598 | 1.00 | 48.95 |
| ATOM | 2899 | C | ILE | 404 | 19.126 | 30.222 | 59.879 | 1.00 | 48.34 |
| ATOM | 2900 | O | ILE | 404 | 19.771 | 29.967 | 60.897 | 1.00 | 48.83 |
| ATOM | 2901 | N | THR | 405 | 18.236 | 29.380 | 59.346 | 1.00 | 46.42 |
| ATOM | 2902 | CA | THR | 405 | 17.956 | 28.043 | 59.892 | 1.00 | 42.37 |
| ATOM | 2903 | CB | THR | 405 | 16.451 | 27.838 | 60.222 | 1.00 | 41.93 |
| ATOM | 2904 | OG1 | THR | 405 | 16.010 | 28.839 | 61.145 | 1.00 | 43.89 |
| ATOM | 2905 | CG2 | THR | 405 | 16.227 | 26.475 | 60.849 | 1.00 | 38.85 |
| ATOM | 2906 | C | THR | 405 | 18.332 | 26.990 | 58.857 | 1.00 | 39.95 |
| ATOM | 2907 | O | THR | 405 | 18.178 | 27.204 | 57.653 | 1.00 | 38.97 |
| ATOM | 2908 | N | VAL | 406 | 18.809 | 25.844 | 59.324 | 1.00 | 38.10 |
| ATOM | 2909 | CA | VAL | 406 | 19.195 | 24.776 | 58.414 | 1.00 | 36.64 |
| ATOM | 2910 | CB | VAL | 406 | 20.686 | 24.442 | 58.563 | 1.00 | 35.12 |
| ATOM | 2911 | CG1 | VAL | 406 | 21.069 | 23.342 | 57.600 | 1.00 | 35.29 |
| ATOM | 2912 | CG2 | VAL | 406 | 21.515 | 25.672 | 58.303 | 1.00 | 35.40 |
| ATOM | 2913 | C | VAL | 406 | 18.390 | 23.499 | 58.635 | 1.00 | 35.83 |
| ATOM | 2914 | O | VAL | 406 | 18.214 | 23.058 | 59.765 | 1.00 | 37.06 |
| ATOM | 2915 | N | GLY | 407 | 17.895 | 22.915 | 57.549 | 1.00 | 34.50 |
| ATOM | 2916 | CA | GLY | 407 | 17.143 | 21.680 | 57.653 | 1.00 | 32.79 |
| ATOM | 2917 | C | GLY | 407 | 18.074 | 20.522 | 57.353 | 1.00 | 32.90 |
| ATOM | 2918 | O | GLY | 407 | 18.704 | 20.467 | 56.294 | 1.00 | 33.85 |
| ATOM | 2919 | N | VAL | 408 | 18.177 | 19.585 | 58.279 | 1.00 | 31.27 |
| ATOM | 2920 | CA | VAL | 408 | 19.064 | 18.466 | 58.054 | 1.00 | 29.57 |
| ATOM | 2921 | CB | VAL | 408 | 20.199 | 18.491 | 59.042 | 1.00 | 29.66 |
| ATOM | 2922 | CG1 | VAL | 408 | 21.390 | 17.767 | 58.468 | 1.00 | 31.77 |
| ATOM | 2923 | CG2 | VAL | 408 | 20.515 | 19.916 | 59.412 | 1.00 | 29.91 |
| ATOM | 2924 | C | VAL | 408 | 18.366 | 17.135 | 58.206 | 1.00 | 29.58 |
| ATOM | 2925 | O | VAL | 408 | 17.392 | 17.015 | 58.942 | 1.00 | 28.54 |
| ATOM | 2926 | N | ASP | 409 | 18.878 | 16.131 | 57.509 | 1.00 | 30.15 |
| ATOM | 2927 | CA | ASP | 409 | 18.324 | 14.789 | 57.598 | 1.00 | 31.95 |
| ATOM | 2928 | CB | ASP | 409 | 17.109 | 14.635 | 56.674 | 1.00 | 35.66 |
| ATOM | 2929 | CG | ASP | 409 | 16.455 | 13.252 | 56.775 | 1.00 | 40.01 |
| ATOM | 2930 | OD1 | ASP | 409 | 15.613 | 12.928 | 55.898 | 1.00 | 40.26 |
| ATOM | 2931 | OD2 | ASP | 409 | 16.773 | 12.499 | 57.728 | 1.00 | 39.33 |
| ATOM | 2932 | C | ASP | 409 | 19.415 | 13.824 | 57.180 | 1.00 | 31.91 |
| ATOM | 2933 | O | ASP | 409 | 20.352 | 14.208 | 56.484 | 1.00 | 32.39 |
| ATOM | 2934 | N | GLY | 410 | 19.300 | 12.574 | 57.607 | 1.00 | 31.09 |
| ATOM | 2935 | CA | GLY | 410 | 20.299 | 11.593 | 57.233 | 1.00 | 29.56 |
| ATOM | 2936 | C | GLY | 410 | 20.703 | 10.704 | 58.385 | 1.00 | 29.32 |
| ATOM | 2937 | O | GLY | 410 | 20.510 | 11.041 | 59.558 | 1.00 | 28.27 |
| ATOM | 2938 | N | SER | 411 | 21.282 | 9.559 | 58.053 | 1.00 | 28.24 |
| ATOM | 2939 | CA | SER | 411 | 21.699 | 8.631 | 59.086 | 1.00 | 27.52 |
| ATOM | 2940 | CB | SER | 411 | 22.018 | 7.253 | 58.481 | 1.00 | 29.46 |
| ATOM | 2941 | OG | SER | 411 | 23.016 | 7.316 | 57.471 | 1.00 | 31.64 |
| ATOM | 2942 | C | SER | 411 | 22.895 | 9.160 | 59.863 | 1.00 | 25.78 |
| ATOM | 2943 | O | SER | 411 | 22.909 | 9.113 | 61.090 | 1.00 | 25.89 |
| ATOM | 2944 | N | VAL | 412 | 23.890 | 9.687 | 59.161 | 1.00 | 23.18 |
| ATOM | 2945 | CA | VAL | 412 | 25.076 | 10.185 | 59.839 | 1.00 | 21.25 |
| ATOM | 2946 | CB | VAL | 412 | 26.099 | 10.669 | 58.841 | 1.00 | 20.56 |
| ATOM | 2947 | CG1 | VAL | 412 | 27.372 | 11.084 | 59.564 | 1.00 | 17.73 |
| ATOM | 2948 | CG2 | VAL | 412 | 26.378 | 9.552 | 57.857 | 1.00 | 20.59 |
| ATOM | 2949 | C | VAL | 412 | 24.769 | 11.300 | 60.818 | 1.00 | 20.44 |
| ATOM | 2950 | O | VAL | 412 | 25.182 | 11.262 | 61.983 | 1.00 | 21.51 |
| ATOM | 2951 | N | TYR | 413 | 24.033 | 12.288 | 60.340 | 1.00 | 16.42 |
| ATOM | 2952 | CA | TYR | 413 | 23.659 | 13.409 | 61.171 | 1.00 | 16.41 |
| ATOM | 2953 | CB | TYR | 413 | 23.095 | 14.526 | 60.288 | 1.00 | 16.40 |
| ATOM | 2954 | CG | TYR | 413 | 22.700 | 15.762 | 61.051 | 1.00 | 14.37 |
| ATOM | 2955 | CD1 | TYR | 413 | 23.645 | 16.707 | 61.434 | 1.00 | 13.43 |
| ATOM | 2956 | CE1 | TYR | 413 | 23.296 | 17.789 | 62.226 | 1.00 | 13.93 |
| ATOM | 2957 | CD2 | TYR | 413 | 21.401 | 15.939 | 61.470 | 1.00 | 13.10 |
| ATOM | 2958 | CE2 | TYR | 413 | 21.049 | 17.007 | 62.256 | 1.00 | 15.82 |
| ATOM | 2959 | CZ | TYR | 413 | 21.994 | 17.927 | 62.638 | 1.00 | 14.65 |
| ATOM | 2960 | OH | TYR | 413 | 21.620 | 18.948 | 63.475 | 1.00 | 16.02 |
| ATOM | 2961 | C | TYR | 413 | 22.626 | 13.007 | 62.233 | 1.00 | 17.41 |
| ATOM | 2962 | O | TYR | 413 | 22.364 | 13.758 | 63.172 | 1.00 | 18.36 |
| ATOM | 2963 | N | LYS | 414 | 22.035 | 11.826 | 62.103 | 1.00 | 18.12 |
| ATOM | 2964 | CA | LYS | 414 | 21.033 | 11.426 | 63.083 | 1.00 | 19.00 |
| ATOM | 2965 | CB | LYS | 414 | 19.706 | 11.130 | 62.384 | 1.00 | 19.22 |
| ATOM | 2966 | CG | LYS | 414 | 18.962 | 12.358 | 61.894 | 1.00 | 18.92 |
| ATOM | 2967 | CD | LYS | 414 | 17.615 | 11.965 | 61.314 | 1.00 | 21.36 |
| ATOM | 2968 | CE | LYS | 414 | 16.829 | 13.181 | 60.855 | 1.00 | 25.08 |
| ATOM | 2969 | NZ | LYS | 414 | 15.567 | 12.829 | 60.132 | 1.00 | 28.46 |
| ATOM | 2970 | C | LYS | 414 | 21.400 | 10.249 | 63.975 | 1.00 | 20.50 |
| ATOM | 2971 | O | LYS | 414 | 20.637 | 9.883 | 64.871 | 1.00 | 21.27 |
| ATOM | 2972 | N | LEU | 415 | 22.565 | 9.655 | 63.753 | 1.00 | 22.22 |
| ATOM | 2973 | CA | LEU | 415 | 22.958 | 8.511 | 64.565 | 1.00 | 23.27 |
| ATOM | 2974 | CB | LEU | 415 | 22.679 | 7.218 | 63.784 | 1.00 | 21.47 |
| ATOM | 2975 | CG | LEU | 415 | 21.234 | 6.978 | 63.313 | 1.00 | 17.45 |
| ATOM | 2976 | CD1 | LEU | 415 | 21.158 | 5.672 | 62.545 | 1.00 | 16.66 |
| ATOM | 2977 | CD2 | LEU | 415 | 20.293 | 6.954 | 64.498 | 1.00 | 14.44 |
| ATOM | 2978 | C | LEU | 415 | 24.418 | 8.566 | 65.033 | 1.00 | 25.05 |
| ATOM | 2979 | O | LEU | 415 | 24.921 | 7.625 | 65.657 | 1.00 | 26.05 |
| ATOM | 2980 | N | HIS | 416 | 25.095 | 9.673 | 64.736 | 1.00 | 24.04 |
| ATOM | 2981 | CA | HIS | 416 | 26.481 | 9.852 | 65.147 | 1.00 | 22.40 |
| ATOM | 2982 | CB | HIS | 416 | 27.365 | 9.997 | 63.922 | 1.00 | 23.29 |
| ATOM | 2983 | CG | HIS | 416 | 27.383 | 8.774 | 63.069 | 1.00 | 25.75 |
| ATOM | 2984 | CD2 | HIS | 416 | 28.392 | 7.937 | 62.729 | 1.00 | 27.82 |
| ATOM | 2985 | ND1 | HIS | 416 | 26.241 | 8.248 | 62.506 | 1.00 | 26.69 |
| ATOM | 2986 | CE1 | HIS | 416 | 26.545 | 7.138 | 61.857 | 1.00 | 28.69 |
| ATOM | 2987 | NE2 | HIS | 416 | 27.844 | 6.926 | 61.977 | 1.00 | 28.20 |
| ATOM | 2988 | C | HIS | 416 | 26.577 | 11.080 | 66.027 | 1.00 | 21.63 |
| ATOM | 2989 | O | HIS | 416 | 26.808 | 12.184 | 65.558 | 1.00 | 22.44 |
| ATOM | 2990 | N | PRO | 417 | 26.386 | 10.898 | 67.331 | 1.00 | 21.25 |
| ATOM | 2991 | CD | PRO | 417 | 26.126 | 9.627 | 68.015 | 1.00 | 22.18 |
| ATOM | 2992 | CA | PRO | 417 | 26.440 | 11.991 | 68.297 | 1.00 | 22.07 |
| ATOM | 2993 | CB | PRO | 417 | 26.447 | 11.258 | 69.627 | 1.00 | 21.52 |
| ATOM | 2994 | CG | PRO | 417 | 25.565 | 10.108 | 69.340 | 1.00 | 23.41 |
| ATOM | 2995 | C | PRO | 417 | 27.655 | 12.874 | 68.113 | 1.00 | 22.46 |
| ATOM | 2996 | O | PRO | 417 | 27.519 | 14.076 | 67.878 | 1.00 | 22.18 |
| ATOM | 2997 | N | SER | 418 | 28.835 | 12.262 | 68.221 | 1.00 | 20.96 |
| ATOM | 2998 | CA | SER | 418 | 30.105 | 12.959 | 68.064 | 1.00 | 18.32 |
| ATOM | 2999 | CB | SER | 418 | 31.264 | 11.962 | 68.076 | 1.00 | 20.88 |
| ATOM | 3000 | OG | SER | 418 | 32.419 | 12.512 | 67.460 | 1.00 | 24.12 |
| ATOM | 3001 | C | SER | 418 | 30.099 | 13.720 | 66.757 | 1.00 | 15.71 |
| ATOM | 3002 | O | SER | 418 | 30.269 | 14.935 | 66.742 | 1.00 | 16.10 |
| ATOM | 3003 | N | PHE | 419 | 29.905 | 13.010 | 65.656 | 1.00 | 11.39 |
| ATOM | 3004 | CA | PHE | 419 | 29.864 | 13.683 | 64.379 | 1.00 | 10.22 |
| ATOM | 3005 | CB | PHE | 419 | 29.243 | 12.789 | 63.335 | 1.00 | 5.53 |
| ATOM | 3006 | CG | PHE | 419 | 29.035 | 13.468 | 62.034 | 1.00 | 1.42 |
| ATOM | 3007 | CD1 | PHE | 419 | 29.814 | 13.137 | 60.942 | 1.00 | 3.13 |
| ATOM | 3008 | CD2 | PHE | 419 | 28.080 | 14.449 | 61.893 | 1.00 | 1.00 |
| ATOM | 3009 | CE1 | PHE | 419 | 29.648 | 13.773 | 59.712 | 1.00 | 1.47 |
| ATOM | 3010 | CE2 | PHE | 419 | 27.909 | 15.088 | 60.670 | 1.00 | 2.68 |
| ATOM | 3011 | CZ | PHE | 419 | 28.699 | 14.746 | 59.575 | 1.00 | 1.00 |
| ATOM | 3012 | C | PHE | 419 | 29.037 | 14.965 | 64.472 | 1.00 | 12.48 |
| ATOM | 3013 | O | PHE | 419 | 29.520 | 16.048 | 64.156 | 1.00 | 12.11 |
| ATOM | 3014 | N | LYS | 420 | 27.785 | 14.838 | 64.900 | 1.00 | 15.88 |
| ATOM | 3015 | CA | LYS | 420 | 26.917 | 16.000 | 64.994 | 1.00 | 20.63 |
| ATOM | 3016 | CB | LYS | 420 | 25.525 | 15.610 | 65.522 | 1.00 | 21.26 |
| ATOM | 3017 | CG | LYS | 420 | 24.470 | 16.730 | 65.361 | 1.00 | 22.35 |
| ATOM | 3018 | CD | LYS | 420 | 23.045 | 16.288 | 65.686 | 1.00 | 22.81 |
| ATOM | 3019 | CE | LYS | 420 | 22.942 | 15.740 | 67.102 | 1.00 | 25.24 |
| ATOM | 3020 | NZ | LYS | 420 | 21.616 | 15.092 | 67.350 | 1.00 | 27.51 |
| ATOM | 3021 | C | LYS | 420 | 27.505 | 17.099 | 65.866 | 1.00 | 24.04 |
| ATOM | 3022 | O | LYS | 420 | 27.533 | 18.260 | 65.465 | 1.00 | 23.74 |
| ATOM | 3023 | N | GLU | 421 | 27.978 | 16.733 | 67.053 | 1.00 | 29.67 |
| ATOM | 3024 | CA | GLU | 421 | 28.550 | 17.701 | 67.999 | 1.00 | 34.96 |
| ATOM | 3025 | CB | GLU | 421 | 29.075 | 16.972 | 69.244 | 1.00 | 36.76 |
| ATOM | 3026 | CG | GLU | 421 | 29.292 | 17.843 | 70.480 | 1.00 | 40.52 |
| ATOM | 3027 | CD | GLU | 421 | 29.895 | 17.047 | 71.638 | 1.00 | 43.55 |
| ATOM | 3028 | OE1 | GLU | 421 | 30.981 | 16.467 | 71.445 | 1.00 | 47.03 |
| ATOM | 3029 | OE2 | GLU | 421 | 29.294 | 16.990 | 72.734 | 1.00 | 43.28 |
| ATOM | 3030 | C | GLU | 421 | 29.680 | 18.512 | 67.369 | 1.00 | 36.40 |
| ATOM | 3031 | O | GLU | 421 | 29.689 | 19.745 | 67.442 | 1.00 | 38.37 |
| ATOM | 3032 | N | ARG | 422 | 30.629 | 17.816 | 66.751 | 1.00 | 35.66 |
| ATOM | 3033 | CA | ARG | 422 | 31.755 | 18.477 | 66.124 | 1.00 | 35.13 |
| ATOM | 3034 | CB | ARG | 422 | 32.801 | 17.449 | 65.684 | 1.00 | 38.76 |
| ATOM | 3035 | CG | ARG | 422 | 33.277 | 16.525 | 66.811 | 1.00 | 46.51 |
| ATOM | 3036 | CD | ARG | 422 | 33.915 | 17.286 | 67.980 | 1.00 | 51.67 |
| ATOM | 3037 | NE | ARG | 422 | 35.322 | 17.578 | 67.732 | 1.00 | 57.41 |
| ATOM | 3038 | CZ | ARG | 422 | 36.269 | 16.649 | 67.625 | 1.00 | 60.70 |
| ATOM | 3039 | NH1 | ARG | 422 | 35.956 | 15.364 | 67.749 | 1.00 | 60.82 |
| ATOM | 3040 | NH2 | ARG | 422 | 37.529 | 17.002 | 67.380 | 1.00 | 61.68 |
| ATOM | 3041 | C | ARG | 422 | 31.256 | 19.278 | 64.942 | 1.00 | 33.47 |
| ATOM | 3042 | O | ARG | 422 | 31.585 | 20.450 | 64.803 | 1.00 | 35.28 |
| ATOM | 3043 | N | PHE | 423 | 30.446 | 18.654 | 64.096 | 1.00 | 32.46 |
| ATOM | 3044 | CA | PHE | 423 | 29.901 | 19.348 | 62.930 | 1.00 | 30.30 |
| ATOM | 3045 | CB | PHE | 423 | 28.949 | 18.423 | 62.165 | 1.00 | 27.32 |
| ATOM | 3046 | CG | PHE | 423 | 28.188 | 19.106 | 61.063 | 1.00 | 23.75 |
| ATOM | 3047 | CD1 | PHE | 423 | 26.891 | 19.552 | 61.270 | 1.00 | 22.33 |
| ATOM | 3048 | CD2 | PHE | 423 | 28.765 | 19.293 | 59.814 | 1.00 | 23.98 |
| ATOM | 3049 | CE1 | PHE | 423 | 26.178 | 20.169 | 60.245 | 1.00 | 22.83 |
| ATOM | 3050 | CE2 | PHE | 423 | 28.061 | 19.909 | 58.784 | 1.00 | 22.46 |
| ATOM | 3051 | CZ | PHE | 423 | 26.769 | 20.347 | 59.001 | 1.00 | 22.73 |
| ATOM | 3052 | C | PHE | 423 | 29.185 | 20.663 | 63.280 | 1.00 | 29.75 |
| ATOM | 3053 | O | PHE | 423 | 29.328 | 21.652 | 62.568 | 1.00 | 27.58 |
| ATOM | 3054 | N | HIS | 424 | 28.415 | 20.694 | 64.363 | 1.00 | 30.19 |
| ATOM | 3055 | CA | HIS | 424 | 27.743 | 21.936 | 64.692 | 1.00 | 32.48 |
| ATOM | 3056 | CB | HIS | 424 | 26.754 | 21.760 | 65.835 | 1.00 | 32.75 |
| ATOM | 3057 | CG | HIS | 424 | 25.412 | 21.279 | 65.387 | 1.00 | 31.94 |
| ATOM | 3058 | CD2 | HIS | 424 | 24.980 | 20.860 | 64.176 | 1.00 | 29.85 |
| ATOM | 3059 | ND1 | HIS | 424 | 24.341 | 21.147 | 66.243 | 1.00 | 32.28 |
| ATOM | 3060 | CE1 | HIS | 424 | 23.308 | 20.661 | 65.580 | 1.00 | 30.67 |
| ATOM | 3061 | NE2 | HIS | 424 | 23.670 | 20.477 | 64.323 | 1.00 | 30.19 |
| ATOM | 3062 | C | HIS | 424 | 28.737 | 23.011 | 65.048 | 1.00 | 35.15 |
| ATOM | 3063 | O | HIS | 424 | 28.689 | 24.102 | 64.487 | 1.00 | 36.91 |
| ATOM | 3064 | N | ALA | 425 | 29.636 | 22.711 | 65.979 | 1.00 | 36.32 |
| ATOM | 3065 | CA | ALA | 425 | 30.652 | 23.675 | 66.395 | 1.00 | 36.74 |
| ATOM | 3066 | CB | ALA | 425 | 31.542 | 23.058 | 67.444 | 1.00 | 35.43 |
| ATOM | 3067 | C | ALA | 425 | 31.492 | 24.149 | 65.201 | 1.00 | 37.82 |
| ATOM | 3068 | O | ALA | 425 | 31.420 | 25.316 | 64.809 | 1.00 | 38.66 |
| ATOM | 3069 | N | SER | 426 | 32.274 | 23.243 | 64.617 | 1.00 | 37.75 |
| ATOM | 3070 | CA | SER | 426 | 33.113 | 23.576 | 63.466 | 1.00 | 37.83 |
| ATOM | 3071 | CB | SER | 426 | 33.602 | 22.289 | 62.782 | 1.00 | 38.67 |
| ATOM | 3072 | OG | SER | 426 | 34.440 | 22.560 | 61.667 | 1.00 | 37.85 |
| ATOM | 3073 | C | SER | 426 | 32.390 | 24.461 | 62.445 | 1.00 | 37.21 |
| ATOM | 3074 | O | SER | 426 | 33.025 | 25.151 | 61.657 | 1.00 | 37.08 |
| ATOM | 3075 | N | VAL | 427 | 31.064 | 24.443 | 62.450 | 1.00 | 37.84 |
| ATOM | 3076 | CA | VAL | 427 | 30.321 | 25.269 | 61.510 | 1.00 | 38.87 |
| ATOM | 3077 | CB | VAL | 427 | 28.935 | 24.667 | 61.194 | 1.00 | 39.38 |
| ATOM | 3078 | CG1 | VAL | 427 | 28.000 | 25.744 | 60.633 | 1.00 | 37.50 |
| ATOM | 3079 | CG2 | VAL | 427 | 29.092 | 23.534 | 60.188 | 1.00 | 36.83 |
| ATOM | 3080 | C | VAL | 427 | 30.138 | 26.655 | 62.090 | 1.00 | 39.54 |
| ATOM | 3081 | O | VAL | 427 | 30.578 | 27.639 | 61.512 | 1.00 | 40.58 |
| ATOM | 3082 | N | ARG | 428 | 29.483 | 26.724 | 63.238 | 1.00 | 40.14 |
| ATOM | 3083 | CA | ARG | 428 | 29.247 | 27.993 | 63.897 | 1.00 | 42.86 |
| ATOM | 3084 | CB | ARG | 428 | 28.603 | 27.739 | 65.258 | 1.00 | 42.72 |
| ATOM | 3085 | CG | ARG | 428 | 27.288 | 26.982 | 65.186 | 1.00 | 43.31 |
| ATOM | 3086 | CD | ARG | 428 | 27.139 | 26.044 | 66.378 | 1.00 | 46.03 |
| ATOM | 3087 | NE | ARG | 428 | 25.802 | 25.461 | 66.485 | 1.00 | 47.83 |
| ATOM | 3088 | CZ | ARG | 428 | 24.690 | 26.173 | 66.648 | 1.00 | 48.39 |
| ATOM | 3089 | NH1 | ARG | 428 | 24.757 | 27.499 | 66.716 | 1.00 | 47.35 |
| ATOM | 3090 | NH2 | ARG | 428 | 23.516 | 25.559 | 66.756 | 1.00 | 47.34 |
| ATOM | 3091 | C | ARG | 428 | 30.561 | 28.768 | 64.064 | 1.00 | 44.67 |
| ATOM | 3092 | O | ARG | 428 | 30.577 | 30.001 | 64.060 | 1.00 | 45.05 |
| ATOM | 3093 | N | ARG | 429 | 31.663 | 28.037 | 64.195 | 1.00 | 45.77 |
| ATOM | 3094 | CA | ARG | 429 | 32.972 | 28.652 | 64.378 | 1.00 | 46.48 |
| ATOM | 3095 | CB | ARG | 429 | 33.849 | 27.738 | 65.244 | 1.00 | 52.63 |
| ATOM | 3096 | CG | ARG | 429 | 33.260 | 27.471 | 66.648 | 1.00 | 59.36 |
| ATOM | 3097 | CD | ARG | 429 | 33.828 | 26.199 | 67.328 | 1.00 | 64.53 |
| ATOM | 3098 | NE | ARG | 429 | 35.247 | 26.286 | 67.677 | 1.00 | 66.23 |
| ATOM | 3099 | CZ | ARG | 429 | 35.963 | 25.274 | 68.159 | 1.00 | 66.80 |
| ATOM | 3100 | NH1 | ARG | 429 | 35.398 | 24.083 | 68.357 | 1.00 | 66.02 |
| ATOM | 3101 | NH2 | ARG | 429 | 37.249 | 25.455 | 68.435 | 1.00 | 68.38 |
| ATOM | 3102 | C | ARG | 429 | 33.657 | 28.954 | 63.049 | 1.00 | 44.06 |
| ATOM | 3103 | O | ARG | 429 | 34.885 | 28.943 | 62.954 | 1.00 | 43.92 |
| ATOM | 3104 | N | LEU | 430 | 32.847 | 29.221 | 62.029 | 1.00 | 41.46 |
| ATOM | 3105 | CA | LEU | 430 | 33.333 | 29.551 | 60.692 | 1.00 | 40.12 |
| ATOM | 3106 | CB | LEU | 430 | 33.495 | 28.300 | 59.830 | 1.00 | 35.57 |
| ATOM | 3107 | CG | LEU | 430 | 34.755 | 27.468 | 60.042 | 1.00 | 34.48 |
| ATOM | 3108 | CD1 | LEU | 430 | 34.764 | 26.279 | 59.101 | 1.00 | 32.77 |
| ATOM | 3109 | CD2 | LEU | 430 | 35.965 | 28.332 | 59.806 | 1.00 | 33.57 |
| ATOM | 3110 | C | LEU | 430 | 32.332 | 30.468 | 60.029 | 1.00 | 42.10 |
| ATOM | 3111 | O | LEU | 430 | 32.503 | 30.868 | 58.880 | 1.00 | 42.67 |
| ATOM | 3112 | N | THR | 431 | 31.280 | 30.797 | 60.763 | 1.00 | 44.70 |
| ATOM | 3113 | CA | THR | 431 | 30.238 | 31.658 | 60.239 | 1.00 | 48.98 |
| ATOM | 3114 | CB | THR | 431 | 28.923 | 30.928 | 60.113 | 1.00 | 49.80 |
| ATOM | 3115 | OG1 | THR | 431 | 28.533 | 30.463 | 61.410 | 1.00 | 50.69 |
| ATOM | 3116 | CG2 | THR | 431 | 29.048 | 29.758 | 59.159 | 1.00 | 51.11 |
| ATOM | 3117 | C | THR | 431 | 29.999 | 32.820 | 61.174 | 1.00 | 51.66 |
| ATOM | 3118 | O | THR | 431 | 28.986 | 32.868 | 61.881 | 1.00 | 52.07 |
| ATOM | 3119 | N | PRO | 432 | 30.935 | 33.774 | 61.190 | 1.00 | 52.95 |
| ATOM | 3120 | CD | PRO | 432 | 32.179 | 33.719 | 60.403 | 1.00 | 51.90 |
| ATOM | 3121 | CA | PRO | 432 | 30.886 | 34.980 | 62.020 | 1.00 | 52.47 |
| ATOM | 3122 | CB | PRO | 432 | 32.135 | 35.733 | 61.587 | 1.00 | 54.48 |
| ATOM | 3123 | CG | PRO | 432 | 33.073 | 34.623 | 61.176 | 1.00 | 54.21 |
| ATOM | 3124 | C | PRO | 432 | 29.620 | 35.783 | 61.739 | 1.00 | 52.15 |
| ATOM | 3125 | O | PRO | 432 | 29.257 | 35.981 | 60.582 | 1.00 | 49.70 |
| ATOM | 3126 | N | SER | 433 | 28.955 | 36.243 | 62.793 | 1.00 | 53.82 |
| ATOM | 3127 | CA | SER | 433 | 27.734 | 37.042 | 62.652 | 1.00 | 57.56 |
| ATOM | 3128 | CB | SER | 433 | 28.055 | 38.372 | 61.952 | 1.00 | 59.89 |
| ATOM | 3129 | OG | SER | 433 | 28.537 | 38.176 | 60.633 | 1.00 | 62.34 |
| ATOM | 3130 | C | SER | 433 | 26.570 | 36.340 | 61.926 | 1.00 | 57.57 |
| ATOM | 3131 | O | SER | 433 | 25.907 | 36.923 | 61.056 | 1.00 | 57.63 |
| ATOM | 3132 | N | CYS | 434 | 26.327 | 35.088 | 62.306 | 1.00 | 56.67 |
| ATOM | 3133 | CA | CYS | 434 | 25.256 | 34.275 | 61.738 | 1.00 | 54.67 |
| ATOM | 3134 | CB | CYS | 434 | 25.805 | 33.375 | 60.634 | 1.00 | 54.21 |
| ATOM | 3135 | SG | CYS | 434 | 26.729 | 34.213 | 59.354 | 1.00 | 55.95 |
| ATOM | 3136 | C | CYS | 434 | 24.657 | 33.390 | 62.832 | 1.00 | 54.71 |
| ATOM | 3137 | O | CYS | 434 | 25.381 | 32.663 | 63.513 | 1.00 | 54.74 |
| ATOM | 3138 | N | GLU | 435 | 23.344 | 33.454 | 63.011 | 1.00 | 54.63 |
| ATOM | 3139 | CA | GLU | 435 | 22.681 | 32.621 | 64.009 | 1.00 | 54.57 |
| ATOM | 3140 | CB | GLU | 435 | 21.529 | 33.383 | 64.681 | 1.00 | 59.73 |
| ATOM | 3141 | CG | GLU | 435 | 21.927 | 34.615 | 65.511 | 1.00 | 64.17 |
| ATOM | 3142 | CD | GLU | 435 | 20.717 | 35.479 | 65.902 | 1.00 | 67.96 |
| ATOM | 3143 | OE1 | GLU | 435 | 20.905 | 36.554 | 66.521 | 1.00 | 68.24 |
| ATOM | 3144 | OE2 | GLU | 435 | 19.574 | 35.079 | 65.584 | 1.00 | 69.98 |
| ATOM | 3145 | C | GLU | 435 | 22.134 | 31.378 | 63.289 | 1.00 | 51.70 |
| ATOM | 3146 | O | GLU | 435 | 21.058 | 31.412 | 62.685 | 1.00 | 51.14 |
| ATOM | 3147 | N | ILE | 436 | 22.889 | 30.288 | 63.350 | 1.00 | 47.61 |
| ATOM | 3148 | CA | ILE | 436 | 22.497 | 29.046 | 62.702 | 1.00 | 43.09 |
| ATOM | 3149 | CB | ILE | 436 | 23.719 | 28.331 | 62.118 | 1.00 | 38.65 |
| ATOM | 3150 | CG2 | ILE | 436 | 23.278 | 27.138 | 61.300 | 1.00 | 38.13 |
| ATOM | 3151 | CG1 | ILE | 436 | 24.502 | 29.286 | 61.234 | 1.00 | 34.79 |
| ATOM | 3152 | CD1 | ILE | 436 | 25.768 | 28.686 | 60.710 | 1.00 | 34.08 |
| ATOM | 3153 | C | ILE | 436 | 21.798 | 28.088 | 63.664 | 1.00 | 42.96 |
| ATOM | 3154 | O | ILE | 436 | 22.403 | 27.608 | 64.621 | 1.00 | 43.46 |
| ATOM | 3155 | N | THR | 437 | 20.521 | 27.821 | 63.402 | 1.00 | 41.73 |
| ATOM | 3156 | CA | THR | 437 | 19.724 | 26.910 | 64.218 | 1.00 | 39.10 |
| ATOM | 3157 | CB | THR | 437 | 18.384 | 27.553 | 64.638 | 1.00 | 37.86 |
| ATOM | 3158 | OG1 | THR | 437 | 18.182 | 28.763 | 63.899 | 1.00 | 37.22 |
| ATOM | 3159 | CG2 | THR | 437 | 18.370 | 27.856 | 66.130 | 1.00 | 37.07 |
| ATOM | 3160 | C | THR | 437 | 19.430 | 25.672 | 63.380 | 1.00 | 38.74 |
| ATOM | 3161 | O | THR | 437 | 18.979 | 25.784 | 62.238 | 1.00 | 39.10 |
| ATOM | 3162 | N | PHE | 438 | 19.696 | 24.494 | 63.936 | 1.00 | 36.24 |
| ATOM | 3163 | CA | PHE | 438 | 19.449 | 23.257 | 63.210 | 1.00 | 33.18 |
| ATOM | 3164 | CB | PHE | 438 | 20.556 | 22.256 | 63.491 | 1.00 | 30.88 |
| ATOM | 3165 | CG | PHE | 438 | 21.905 | 22.742 | 63.093 | 1.00 | 32.48 |
| ATOM | 3166 | CD1 | PHE | 438 | 22.597 | 23.652 | 63.887 | 1.00 | 31.95 |
| ATOM | 3167 | CD2 | PHE | 438 | 22.489 | 22.301 | 61.913 | 1.00 | 32.85 |
| ATOM | 3168 | CE1 | PHE | 438 | 23.857 | 24.118 | 63.507 | 1.00 | 31.30 |
| ATOM | 3169 | CE2 | PHE | 438 | 23.745 | 22.758 | 61.522 | 1.00 | 32.28 |
| ATOM | 3170 | CZ | PHE | 438 | 24.432 | 23.668 | 62.320 | 1.00 | 31.80 |
| ATOM | 3171 | C | PHE | 438 | 18.102 | 22.648 | 63.563 | 1.00 | 33.15 |
| ATOM | 3172 | O | PHE | 438 | 17.662 | 22.729 | 64.705 | 1.00 | 34.90 |
| ATOM | 3173 | N | ILE | 439 | 17.450 | 22.049 | 62.570 | 1.00 | 31.06 |
| ATOM | 3174 | CA | ILE | 439 | 16.150 | 21.412 | 62.738 | 1.00 | 28.59 |
| ATOM | 3175 | CB | ILE | 439 | 15.010 | 22.347 | 62.321 | 1.00 | 26.74 |
| ATOM | 3176 | CG2 | ILE | 439 | 15.268 | 22.879 | 60.937 | 1.00 | 27.91 |
| ATOM | 3177 | CG1 | ILE | 439 | 13.683 | 21.591 | 62.312 | 1.00 | 27.91 |
| ATOM | 3178 | CD1 | ILE | 439 | 12.545 | 22.406 | 61.776 | 1.00 | 26.70 |
| ATOM | 3179 | C | ILE | 439 | 16.113 | 20.190 | 61.837 | 1.00 | 29.76 |
| ATOM | 3180 | O | ILE | 439 | 16.208 | 20.310 | 60.618 | 1.00 | 29.55 |
| ATOM | 3181 | N | GLU | 440 | 15.977 | 19.014 | 62.434 | 1.00 | 30.76 |
| ATOM | 3182 | CA | GLU | 440 | 15.934 | 17.781 | 61.666 | 1.00 | 32.34 |
| ATOM | 3183 | CB | GLU | 440 | 16.028 | 16.592 | 62.609 | 1.00 | 34.09 |
| ATOM | 3184 | CG | GLU | 440 | 17.272 | 16.583 | 63.458 | 1.00 | 38.93 |
| ATOM | 3185 | CD | GLU | 440 | 17.339 | 15.367 | 64.353 | 1.00 | 43.10 |
| ATOM | 3186 | OE1 | GLU | 440 | 16.378 | 15.162 | 65.131 | 1.00 | 44.22 |
| ATOM | 3187 | OE2 | GLU | 440 | 18.346 | 14.623 | 64.277 | 1.00 | 44.99 |
| ATOM | 3188 | C | GLU | 440 | 14.648 | 17.687 | 60.854 | 1.00 | 33.11 |
| ATOM | 3189 | O | GLU | 440 | 13.703 | 18.445 | 61.086 | 1.00 | 31.25 |
| ATOM | 3190 | N | SER | 441 | 14.613 | 16.764 | 59.896 | 1.00 | 34.70 |
| ATOM | 3191 | CA | SER | 441 | 13.416 | 16.587 | 59.086 | 1.00 | 37.07 |
| ATOM | 3192 | CB | SER | 441 | 13.738 | 15.904 | 57.761 | 1.00 | 34.79 |
| ATOM | 3193 | OG | SER | 441 | 14.159 | 14.579 | 57.988 | 1.00 | 34.61 |
| ATOM | 3194 | C | SER | 441 | 12.452 | 15.724 | 59.889 | 1.00 | 40.43 |
| ATOM | 3195 | O | SER | 441 | 12.866 | 14.964 | 60.773 | 1.00 | 38.99 |
| ATOM | 3196 | N | GLU | 442 | 11.168 | 15.855 | 59.571 | 1.00 | 43.31 |
| ATOM | 3197 | CA | GLU | 442 | 10.099 | 15.135 | 60.254 | 1.00 | 45.59 |
| ATOM | 3198 | CB | GLU | 442 | 8.764 | 15.638 | 59.724 | 1.00 | 46.37 |
| ATOM | 3199 | CG | GLU | 442 | 7.575 | 15.244 | 60.549 | 1.00 | 49.47 |
| ATOM | 3200 | CD | GLU | 442 | 6.653 | 16.421 | 60.794 | 1.00 | 52.25 |
| ATOM | 3201 | OE1 | GLU | 442 | 5.425 | 16.199 | 60.895 | 1.00 | 52.44 |
| ATOM | 3202 | OE2 | GLU | 442 | 7.160 | 17.568 | 60.894 | 1.00 | 53.16 |
| ATOM | 3203 | C | GLU | 442 | 10.165 | 13.607 | 60.174 | 1.00 | 47.69 |
| ATOM | 3204 | O | GLU | 442 | 10.828 | 13.035 | 59.314 | 1.00 | 47.21 |
| ATOM | 3205 | N | GLU | 443 | 9.435 | 12.964 | 61.076 | 1.00 | 50.15 |
| ATOM | 3206 | CA | GLU | 443 | 9.382 | 11.508 | 61.210 | 1.00 | 52.79 |
| ATOM | 3207 | CB | GLU | 443 | 8.911 | 11.204 | 62.623 | 1.00 | 55.86 |
| ATOM | 3208 | CG | GLU | 443 | 9.468 | 12.183 | 63.635 | 1.00 | 61.71 |
| ATOM | 3209 | CD | GLU | 443 | 10.948 | 11.962 | 63.877 | 1.00 | 66.19 |
| ATOM | 3210 | OE1 | GLU | 443 | 11.689 | 11.746 | 62.886 | 1.00 | 68.22 |
| ATOM | 3211 | OE2 | GLU | 443 | 11.365 | 12.006 | 65.058 | 1.00 | 67.57 |
| ATOM | 3212 | C | GLU | 443 | 8.521 | 10.711 | 60.218 | 1.00 | 53.03 |
| ATOM | 3213 | O | GLU | 443 | 7.344 | 10.999 | 60.025 | 1.00 | 54.82 |
| ATOM | 3214 | N | GLY | 444 | 9.116 | 9.702 | 59.593 | 1.00 | 52.26 |
| ATOM | 3215 | CA | GLY | 444 | 8.373 | 8.861 | 58.667 | 1.00 | 52.32 |
| ATOM | 3216 | C | GLY | 444 | 7.966 | 9.389 | 57.302 | 1.00 | 53.07 |
| ATOM | 3217 | O | GLY | 444 | 6.767 | 9.460 | 57.003 | 1.00 | 52.97 |
| ATOM | 3218 | N | SER | 445 | 8.961 | 9.750 | 56.483 | 1.00 | 52.71 |
| ATOM | 3219 | CA | SER | 445 | 8.760 | 10.239 | 55.104 | 1.00 | 50.05 |
| ATOM | 3220 | CB | SER | 445 | 7.836 | 11.464 | 55.084 | 1.00 | 51.01 |
| ATOM | 3221 | OG | SER | 445 | 6.487 | 11.084 | 55.318 | 1.00 | 46.84 |
| ATOM | 3222 | C | SER | 445 | 10.076 | 10.545 | 54.356 | 1.00 | 46.51 |
| ATOM | 3223 | O | SER | 445 | 11.123 | 10.710 | 54.976 | 1.00 | 45.30 |
| ATOM | 3224 | N | GLY | 446 | 10.013 | 10.603 | 53.026 | 1.00 | 43.17 |
| ATOM | 3225 | CA | GLY | 446 | 11.207 | 10.842 | 52.235 | 1.00 | 40.08 |
| ATOM | 3226 | C | GLY | 446 | 11.199 | 12.057 | 51.330 | 1.00 | 39.05 |
| ATOM | 3227 | O | GLY | 446 | 11.414 | 13.164 | 51.803 | 1.00 | 42.74 |
| ATOM | 3228 | N | ARG | 447 | 10.940 | 11.873 | 50.039 | 1.00 | 37.60 |
| ATOM | 3229 | CA | ARG | 447 | 10.956 | 13.000 | 49.098 | 1.00 | 37.46 |
| ATOM | 3230 | CB | ARG | 447 | 11.549 | 12.546 | 47.747 | 1.00 | 45.51 |
| ATOM | 3231 | CG | ARG | 447 | 10.793 | 11.401 | 47.014 | 1.00 | 53.91 |
| ATOM | 3232 | CD | ARG | 447 | 11.521 | 10.902 | 45.743 | 1.00 | 58.18 |
| ATOM | 3233 | NE | ARG | 447 | 12.939 | 10.598 | 45.975 | 1.00 | 63.62 |
| ATOM | 3234 | CZ | ARG | 447 | 13.399 | 9.623 | 46.769 | 1.00 | 66.02 |
| ATOM | 3235 | NH1 | ARG | 447 | 12.560 | 8.825 | 47.425 | 1.00 | 67.97 |
| ATOM | 3236 | NH2 | ARG | 447 | 14.711 | 9.447 | 46.924 | 1.00 | 69.08 |
| ATOM | 3237 | C | ARG | 447 | 9.642 | 13.737 | 48.848 | 1.00 | 32.93 |
| ATOM | 3238 | O | ARG | 447 | 9.122 | 14.416 | 49.741 | 1.00 | 29.46 |
| ATOM | 3239 | N | GLY | 448 | 9.150 | 13.625 | 47.607 | 1.00 | 30.52 |
| ATOM | 3240 | CA | GLY | 448 | 7.902 | 14.245 | 47.202 | 1.00 | 26.03 |
| ATOM | 3241 | C | GLY | 448 | 6.845 | 13.828 | 48.200 | 1.00 | 25.25 |
| ATOM | 3242 | O | GLY | 448 | 5.752 | 14.378 | 48.244 | 1.00 | 24.59 |
| ATOM | 3243 | N | ALA | 449 | 7.186 | 12.840 | 49.018 | 1.00 | 23.74 |
| ATOM | 3244 | CA | ALA | 449 | 6.282 | 12.362 | 50.035 | 1.00 | 20.54 |
| ATOM | 3245 | CB | ALA | 449 | 6.611 | 10.917 | 50.410 | 1.00 | 18.12 |
| ATOM | 3246 | C | ALA | 449 | 6.340 | 13.251 | 51.282 | 1.00 | 22.37 |
| ATOM | 3247 | O | ALA | 449 | 5.307 | 13.782 | 51.693 | 1.00 | 22.56 |
| ATOM | 3248 | N | ALA | 450 | 7.524 | 13.443 | 51.881 | 1.00 | 22.28 |
| ATOM | 3249 | CA | ALA | 450 | 7.605 | 14.261 | 53.088 | 1.00 | 20.98 |
| ATOM | 3250 | CB | ALA | 450 | 9.056 | 14.432 | 53.535 | 1.00 | 9.63 |
| ATOM | 3251 | C | ALA | 450 | 6.937 | 15.594 | 52.872 | 1.00 | 20.66 |
| ATOM | 3252 | O | ALA | 450 | 6.417 | 16.168 | 53.826 | 1.00 | 19.81 |
| ATOM | 3253 | N | LEU | 451 | 6.943 | 16.109 | 51.702 | 1.00 | 19.91 |
| ATOM | 3254 | CA | LEU | 451 | 6.279 | 17.379 | 51.602 | 1.00 | 22.22 |
| ATOM | 3255 | CB | LEU | 451 | 6.586 | 18.056 | 50.279 | 1.00 | 26.74 |
| ATOM | 3256 | CG | LEU | 451 | 6.089 | 19.496 | 50.144 | 1.00 | 34.07 |
| ATOM | 3257 | CD1 | LEU | 451 | 6.894 | 20.425 | 51.040 | 1.00 | 37.55 |
| ATOM | 3258 | CD2 | LEU | 451 | 6.160 | 19.955 | 48.696 | 1.00 | 36.19 |
| ATOM | 3259 | C | LEU | 451 | 4.774 | 17.157 | 51.686 | 1.00 | 23.24 |
| ATOM | 3260 | O | LEU | 451 | 4.136 | 17.474 | 52.699 | 1.00 | 21.18 |
| ATOM | 3261 | N | VAL | 452 | 4.212 | 16.613 | 50.608 | 1.00 | 26.44 |
| ATOM | 3262 | CA | VAL | 452 | 2.798 | 16.405 | 50.557 | 1.00 | 26.90 |
| ATOM | 3263 | CB | VAL | 452 | 2.454 | 15.179 | 49.666 | 1.00 | 28.65 |
| ATOM | 3264 | CG1 | VAL | 452 | 3.082 | 15.353 | 48.300 | 1.00 | 26.01 |
| ATOM | 3265 | CG2 | VAL | 452 | 2.933 | 13.886 | 50.313 | 1.00 | 31.22 |
| ATOM | 3266 | C | VAL | 452 | 2.217 | 16.308 | 51.935 | 1.00 | 26.69 |
| ATOM | 3267 | O | VAL | 452 | 1.181 | 16.878 | 52.234 | 1.00 | 24.86 |
| ATOM | 3268 | N | SER | 453 | 2.880 | 15.579 | 52.769 | 1.00 | 26.07 |
| ATOM | 3269 | CA | SER | 453 | 2.377 | 15.346 | 54.125 | 1.00 | 26.79 |
| ATOM | 3270 | CB | SER | 453 | 3.058 | 14.127 | 54.756 | 1.00 | 28.88 |
| ATOM | 3271 | OG | SER | 453 | 2.553 | 13.881 | 56.057 | 1.00 | 37.16 |
| ATOM | 3272 | C | SER | 453 | 2.571 | 16.558 | 55.028 | 1.00 | 26.82 |
| ATOM | 3273 | O | SER | 453 | 1.658 | 17.301 | 55.377 | 1.00 | 26.28 |
| ATOM | 3274 | N | ALA | 454 | 3.831 | 16.701 | 55.389 | 1.00 | 26.83 |
| ATOM | 3275 | CA | ALA | 454 | 4.250 | 17.807 | 56.177 | 1.00 | 23.37 |
| ATOM | 3276 | CB | ALA | 454 | 5.719 | 18.128 | 55.937 | 1.00 | 17.54 |
| ATOM | 3277 | C | ALA | 454 | 3.381 | 19.002 | 55.866 | 1.00 | 21.80 |
| ATOM | 3278 | O | ALA | 454 | 3.194 | 19.883 | 56.714 | 1.00 | 21.02 |
| ATOM | 3279 | N | VAL | 455 | 2.848 | 19.044 | 54.656 | 1.00 | 21.59 |
| ATOM | 3280 | CA | VAL | 455 | 2.020 | 20.156 | 54.281 | 1.00 | 25.32 |
| ATOM | 3281 | CB | VAL | 455 | 2.313 | 20.557 | 52.841 | 1.00 | 28.03 |
| ATOM | 3282 | CG1 | VAL | 455 | 1.676 | 19.583 | 51.871 | 1.00 | 29.06 |
| ATOM | 3283 | CG2 | VAL | 455 | 1.813 | 21.961 | 52.577 | 1.00 | 29.98 |
| ATOM | 3284 | C | VAL | 455 | 0.528 | 19.890 | 54.469 | 1.00 | 27.69 |
| ATOM | 3285 | O | VAL | 455 | −0.202 | 20.783 | 54.911 | 1.00 | 28.10 |
| ATOM | 3286 | N | ALA | 456 | 0.061 | 18.681 | 54.142 | 1.00 | 30.51 |
| ATOM | 3287 | CA | ALA | 456 | −1.367 | 18.349 | 54.318 | 1.00 | 31.54 |
| ATOM | 3288 | CB | ALA | 456 | −1.666 | 16.937 | 53.836 | 1.00 | 25.20 |
| ATOM | 3289 | C | ALA | 456 | −1.702 | 18.505 | 55.797 | 1.00 | 31.77 |
| ATOM | 3290 | O | ALA | 456 | −2.853 | 18.713 | 56.176 | 1.00 | 33.11 |
| ATOM | 3291 | N | CYS | 457 | −0.673 | 18.384 | 56.625 | 1.00 | 31.33 |
| ATOM | 3292 | CA | CYS | 457 | −0.843 | 18.538 | 58.049 | 1.00 | 33.33 |
| ATOM | 3293 | CB | CYS | 457 | 0.262 | 17.815 | 58.811 | 1.00 | 36.53 |
| ATOM | 3294 | SG | CYS | 457 | 1.040 | 16.448 | 57.890 | 1.00 | 44.65 |
| ATOM | 3295 | C | CYS | 457 | −0.903 | 19.990 | 58.438 | 1.00 | 34.59 |
| ATOM | 3296 | O | CYS | 457 | −1.745 | 20.391 | 59.237 | 1.00 | 34.67 |
| ATOM | 3297 | N | LYS | 458 | 0.005 | 20.779 | 57.881 | 1.00 | 37.14 |
| ATOM | 3298 | CA | LYS | 458 | 0.060 | 22.199 | 58.190 | 1.00 | 38.61 |
| ATOM | 3299 | CB | LYS | 458 | 1.363 | 22.799 | 57.669 | 1.00 | 37.21 |
| ATOM | 3300 | CG | LYS | 458 | 2.573 | 22.474 | 58.538 | 1.00 | 37.81 |
| ATOM | 3301 | CD | LYS | 458 | 2.501 | 23.206 | 59.874 | 1.00 | 38.84 |
| ATOM | 3302 | CE | LYS | 458 | 3.820 | 23.143 | 60.639 | 1.00 | 38.18 |
| ATOM | 3303 | NZ | LYS | 458 | 3.812 | 24.023 | 61.851 | 1.00 | 36.51 |
| ATOM | 3304 | C | LYS | 458 | −1.128 | 22.920 | 57.596 | 1.00 | 40.24 |
| ATOM | 3305 | O | LYS | 458 | −1.377 | 24.079 | 57.898 | 1.00 | 39.64 |
| ATOM | 3306 | N | LYS | 459 | −1.869 | 22.223 | 56.752 | 1.00 | 43.69 |
| ATOM | 3307 | CA | LYS | 459 | −3.036 | 22.820 | 56.147 | 1.00 | 50.66 |
| ATOM | 3308 | CB | LYS | 459 | −3.242 | 22.248 | 54.747 | 1.00 | 55.88 |
| ATOM | 3309 | CG | LYS | 459 | −4.657 | 22.405 | 54.183 | 1.00 | 63.64 |
| ATOM | 3310 | CD | LYS | 459 | −5.037 | 23.850 | 53.856 | 1.00 | 66.97 |
| ATOM | 3311 | CE | LYS | 459 | −6.431 | 23.941 | 53.226 | 1.00 | 68.86 |
| ATOM | 3312 | NZ | LYS | 459 | −7.531 | 23.519 | 54.152 | 1.00 | 71.25 |
| ATOM | 3313 | C | LYS | 459 | −4.262 | 22.562 | 57.018 | 1.00 | 52.41 |
| ATOM | 3314 | O | LYS | 459 | −5.132 | 23.425 | 57.132 | 1.00 | 51.90 |
| ATOM | 3315 | N | ALA | 460 | −4.322 | 21.380 | 57.634 | 1.00 | 54.96 |
| ATOM | 3316 | CA | ALA | 460 | −5.449 | 20.997 | 58.495 | 1.00 | 57.72 |
| ATOM | 3317 | CB | ALA | 460 | −5.201 | 19.620 | 59.111 | 1.00 | 54.90 |
| ATOM | 3318 | C | ALA | 460 | −5.736 | 22.018 | 59.596 | 1.00 | 60.41 |
| ATOM | 3319 | O | ALA | 460 | −6.773 | 21.950 | 60.261 | 1.00 | 60.54 |
| ATOM | 3320 | N | CYS | 461 | −4.815 | 22.965 | 59.776 | 1.00 | 63.50 |
| ATOM | 3321 | CA | CYS | 461 | −4.961 | 24.022 | 60.776 | 1.00 | 66.18 |
| ATOM | 3322 | CB | CYS | 461 | −3.580 | 24.489 | 61.252 | 1.00 | 67.98 |
| ATOM | 3323 | SG | CYS | 461 | −3.604 | 26.041 | 62.185 | 1.00 | 75.61 |
| ATOM | 3324 | C | CYS | 461 | −5.727 | 25.217 | 60.200 | 1.00 | 65.92 |
| ATOM | 3325 | O | CYS | 461 | −6.940 | 25.348 | 60.490 | 1.00 | 65.70 |
| ATOM | 3326 | OXT | CYS | 461 | −5.099 | 26.001 | 59.454 | 1.00 | 65.20 |
| ATOM | 3327 | S | SO4 | 600 | 20.241 | 7.477 | 54.655 | 1.00 | 35.04 |
| ATOM | 3328 | O1 | SO4 | 600 | 19.370 | 7.951 | 53.566 | 1.00 | 33.14 |
| ATOM | 3329 | O2 | SO4 | 600 | 20.343 | 8.532 | 55.683 | 1.00 | 32.80 |
| ATOM | 3330 | O3 | SO4 | 600 | 19.690 | 6.249 | 55.260 | 1.00 | 33.32 |
| ATOM | 3331 | O4 | SO4 | 600 | 21.572 | 7.178 | 54.108 | 1.00 | 33.97 |
| ATOM | 3332 | S | SO4 | 601 | 22.953 | 22.471 | 69.199 | 1.00 | 77.32 |
| ATOM | 3333 | O1 | SO4 | 601 | 21.971 | 21.759 | 68.356 | 1.00 | 76.19 |
| ATOM | 3334 | O2 | SO4 | 601 | 22.411 | 23.803 | 69.553 | 1.00 | 77.48 |
| ATOM | 3335 | O3 | SO4 | 601 | 23.205 | 21.698 | 70.433 | 1.00 | 77.23 |
| ATOM | 3336 | O4 | SO4 | 601 | 24.224 | 22.628 | 68.461 | 1.00 | 77.19 |
| ATOM | 3337 | NA + 1 | NA1 | 602 | 17.158 | 10.244 | 54.280 | 1.00 | 10.17 |
| ATOM | 3338 | OH2 | HOH | 603 | 19.770 | 14.543 | 47.159 | 1.00 | 1.00 |
| ATOM | 3340 | OH2 | HOH | 604 | 20.723 | 24.387 | 67.178 | 1.00 | 17.94 |
| ATOM | 3341 | OH2 | HOH | 605 | 10.880 | 33.802 | 37.628 | 1.00 | 1.00 |
| ATOM | 3342 | OH2 | HOH | 606 | 22.743 | 28.762 | 37.147 | 1.00 | 31.78 |
| ATOM | 3343 | OH2 | HOH | 607 | 38.906 | 1.328 | 74.611 | 1.00 | 37.76 |
| ATOM | 3344 | OH2 | HOH | 608 | 1.237 | 30.510 | 46.162 | 1.00 | 32.40 |
| ATOM | 3345 | OH2 | HOH | 609 | 34.702 | −1.731 | 56.455 | 1.00 | 62.03 |
| END | | | | | | | | | |

Table 2 is made according to a description method of Protein Data Bank conventionally used by those skilled in the art. In Table 2, HOH represents a water molecule.

In the present invention, a crystal of a protein having substantially the same amino acid sequence as that of SEQ ID No. 5 and/or SEQ ID No. 8 and having a glucokinase activity is within the range of the present invention. Mentioned as such a crystal are, for example, crystals wherein, in three dimensional structure coordinate data obtained by changing at least one datum in three dimensional structure coordinate data described in Table 1 and/or Table 2, the average square deviation between an atom (Cα atom) of a main chain of an amino acid shown by three dimensional structure coordinate data described in Table 1 and/or Table 2 and a Cα atom shown by the above-mentioned changed three dimensional structure coordinate data corresponding to the Cα atom is 0.6 Å or less. Even if the numerical value of a coordinate representing the position of an atom varies, two structure coordinates containing corresponding atoms of which positions can be overlapped represent the same three dimensional structure.

The three dimensional structure coordinates of a GK protein described in Table 1 and/or Table 2 are important information for drug design, and if necessary, stored in a storage medium readable by a computer, and this information is treated by a computer to perform drug design. Therefore, according an another embodiment of the present invention, there is provided a computer-readable storage medium containing a recorded program for allowing a computer to function as a three dimensional coordinate memory means for storing three dimensional coordinates of amino acid residues described in Table 1 and/or Table 2.

According an another embodiment of the present invention, there is provided a computer-readable recording medium containing a recorded program for allowing a computer to function as a three dimensional coordinate memory means storing information regarding three dimensional coordinates of amino acid residues described in Table 1 and/or Table 2, as a binding portion guessing means for guessing a compound binding portion of a protein having an amino acid sequence as depicted in SEQ ID No. 5 and/or SEQ ID No. 8 using three dimensional coordinates of amino acid residues described in Table 1 and/or Table 2 stored in the above-mentioned three dimensional coordinate memory means, as a binding compound memory means memorizing information regarding the kind of a compound connected to a protein and the three dimensional structure of the compound, and as a binding compound candidate selection means for selecting candidates of a compound suitable for a compound binding portion of a protein having an amino acid sequence as depicted in SEQ ID No. 1 of the sequence table, using, at least, information regarding the three dimensional structure of a compound binding portion of a protein having an amino acid sequence as depicted in SEQ ID No. 5 and/or SEQ ID No. 8 guessed by the above-mentioned binding portion guessing means and information regarding the three dimensional structure of a compound stored in the above-mentioned binding compound memory means. Further, according to another embodiment of the present invention, a computer equipped with the above-mentioned means is also provided.

(Crystal of Complex of GK Protein and Compound Binding to this)

Next, according to another embodiment of the present invention, there are provided a crystal containing a complex composed of a protein containing an amino acid sequence as depicted in SEQ ID No. 5 or SEQ ID No. 8 or an amino acid sequence which is substantially the same as this amino acid sequence and of a compound capable of binding to this protein, and a method of producing the crystal.

When a compound binding to a GK protein is obtained, a GK protein and its compound are, first, for example, mixed in an aqueous solution to form a complex. A crystal of such a complex is produced by known co-crystallization methods such as a co-crystallization method, soaking method and the like. Regarding crystallization conditions and crystallization methods, the above-mentioned methods are referred to.

The compound binding to a GK protein is selected, for example, from a group of compounds of the above-described formula (I).

Here, as the halogen atom in the above-described formula (I), a fluorine atom, chlorine atom, bromine atom, iodine atom and the like are exemplified, and of them, a chlorine atom is preferable.

As the substituent on A, B in the above-mentioned formula (I) and a heteroaryl group of the formula (II), there are listed an amino group, carbamoyl group, carbamoylamino group, carbamoyloxy group, carboxyl group, cyano group, sulfamoyl group, trifluoromethyl group, halogen atom, hydroxyl group, formyl group, straight-chain C₁ to C₆ alkyl group, cyclic C₃ to C₆ hydrocarbon group, aralkyl group, N-aralkylamino group, N,N-diaralkylamino group, aralkyloxy group, aralkylcarbonyl group, N-aralkylcarbamoyl group, aryl group, arylthio group, N-arylamino group, aryloxy group, arylsulfonyl group, arylsulfonyloxy group, N-arylsulfonylamino group, arylsulfamoyl group, N-arylcarbamoyl group, aroyl group, aroxy group, C₂ to C₆ alkanoyl group, N—C₂ to C₆ alkanoylamino group, C₁ to C₆ alkylthio group, N—C₁ to C₆ alkylsulfamoyl group, N,N-di-C₁ to C₆ alkylsulfamoyl group, C₁ to C₆ alkylsulfinyl group, C₁ to C₆ alkylsulfonyl group, N—C₁ to C₆ alkylsulfonylamino group, C₁ to C₆ alkoxy group, C₁ to C₆ alkoxycarbonyl group, C₁ to C₆ alkylamino group, and the like. Preferable examples thereof used here include an amino group, carbamoyl group, carbamoylamino group, carbamoyloxy group, carboxyl group, cyano group, sulfamoyl group, trifluoromethyl group, halogen atom, hydroxyl group, formyl group, straight-chain C₁ to C₆ alkyl group and the like.

Here, “hydrocarbon group” means a straight-chain C₁ to C₆ alkyl group, or a group in which 1 or 2, preferably 1 of carbon atoms constituting the alkyl group may be substituted by a nitrogen atom, sulfur atom or oxygen atom and/or carbon atoms in the straight-chain C₁ to C₆ alkyl group may be mutually connected via a double bond or triple bond. The number of the double bond or triple bond is preferably 1 or 2, more preferably 1.

It is preferable that the hydrocarbon group is selected from specifically a methyl group, ethyl group, propyl group, isopropyl group, butyl group and groups of the following formulae. 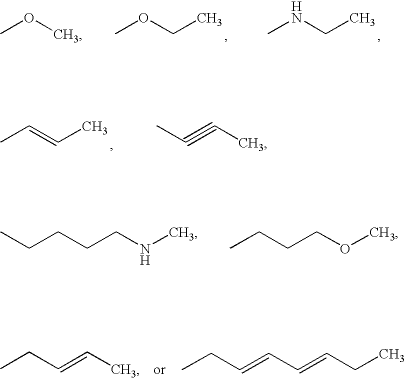
More preferably, the hydrocarbon group is selected from a methyl group, ethyl group, propyl group, isopropyl group and groups of the following formulae.

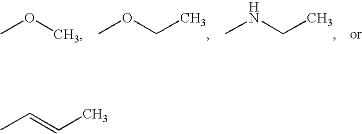

As preferable A (when p=0), the following groups are exemplified.

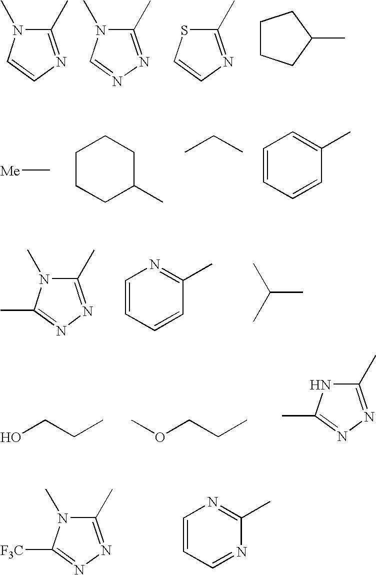

As preferable B, the following groups are exemplified.

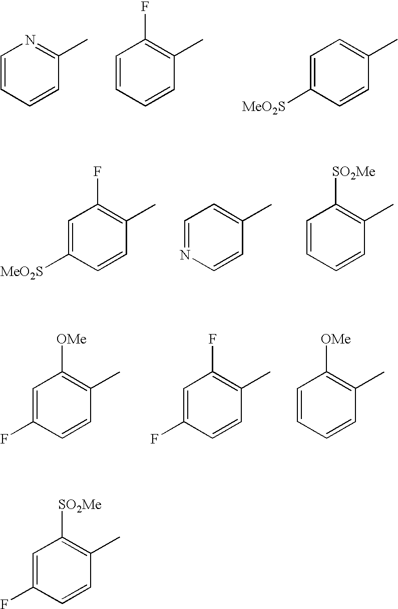

As the heteroaryl group of the formula (II), the following heterocyclic groups are exemplified.

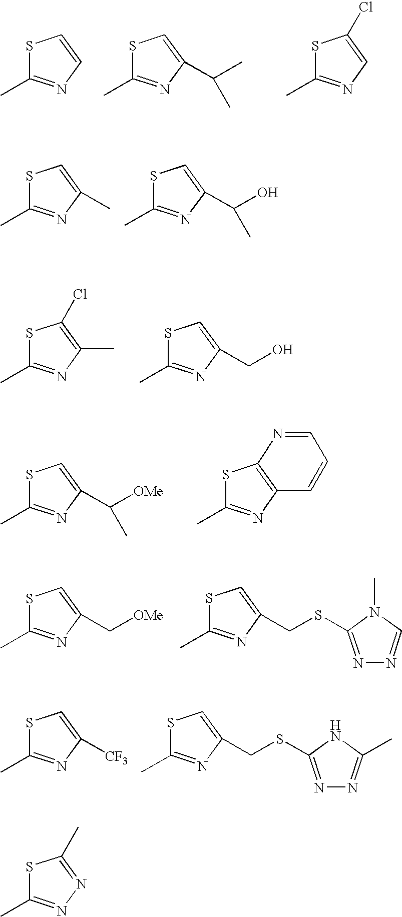

Particularly preferable compounds are compounds of the above-described formulae (IIIa) to (IIIc).

The compound (I) of the present invention can be produced easily using known reaction means or according to known methods. A compound of the general formula (I) of the present invention can be produced not only by usual liquid phase synthesis methods but also by solid phase synthesis methods such as for example combinatorial method, parallel synthesis method and the like showing remarkable development recently. Preferably, the compound (I) can be produced by, for example, the following method. 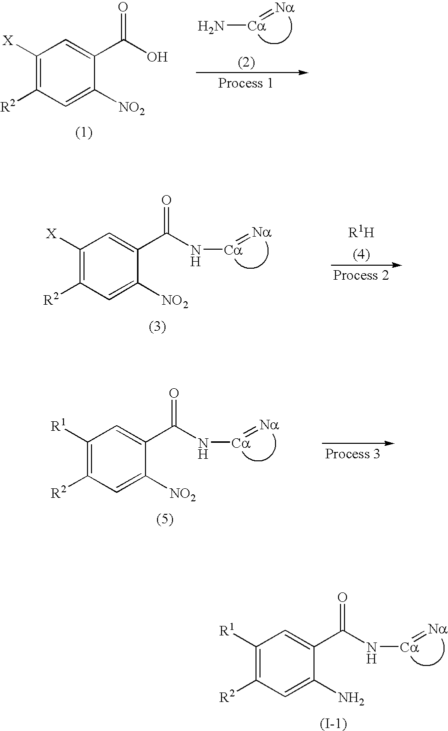
[wherein, marks are as defined above]
(Process 1)

This process is a method in which a carboxylic acid compound (1) or its reactive derivative is reacted with a mono-cyclic or di-cyclic amino compound optionally substituted having a heteroaryl group of the above-mentioned formula (2), to produce a compound (3). In this reaction, a usual amide formation reaction may be advantageously conducted by methods described in literatures (for example, Izumiya Nobuo, et al., Peptide Gosei no Kiso to Jikken, Maruzen, 1983, Comprehensive Organic Synthesis, vol. 6, Pergamon Press, 1991, and the like), methods according to these methods, or combinations of them with ordinary methods, that is, this process can be conducted using a condensing agent well known to those skilled in the art, or by an ester activating method, mixed acid anhydride method, acid chloride method, carbodiimide method and the like utilizable by those skilled in the art. Suitable as such amide forming reagents are, for example, thionyl chloride, N,N-dicyclohexylcarbodiimide, 1-methyl-2-bromopyridinium iodide, N,N′-carbonyl diimidazole, diphenylphosphoryl chloride, diphenylphosphorylazide, N,N′-disuccinimidyl carbonate, N,N′-disuccinimidyl oxalate, 1-ethyl-3-(3-dimethylaminopropyl)carbodiimide hydrochloride, ethyl chloroformate, isobutyl chloroformate, benzotriazo-1-yl-oxy-tris(dimethylamino)phosphonium hexafluorophosphate and the like, and of them, thionyl chloride, N,N-dicyclohexylcarbodiimide, benzotriazo-1-yl-oxy-tris(dimethylamino)phosphonium hexafluorophosphate and the like are preferable. In the amide formation reaction, a base and condensing aid may be used together with the above-mentioned amide formation reagents.

Example of the base used include tertiary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undec-7-ene (DBU), 1,5-azabicyclo[4.3.0]nona-5-ene (DBN) and the like; aromatic amines such as pyridine, 4-dimethylaminopyridine, picoline, lutidine, quinoline, isoquinoline and the like, and of them, for example, tertiary aliphatic amines are preferable, and particularly, triethylamine, N,N-diisopropylethylamine and the like are suitable.

As the condensing aid used, for example, N-hydroxybenzotriazole hydrate, N-hydroxysuccinimide, N-hydroxy-5-norbornene-2,3-dicarboxyimide, 3-hydroxy-3,4,-dihydro-4-oxo-1,2,3-benzotriazole and the like are listed, and of them, N-hydroxybenzotriazole and the like are suitable.

The amount of the amino compound (2) used varies depending on the kinds of a compound and a solvent used and other reaction conditions, and usually, is 0.02 to 50 equivalent, preferably 0.2 to 2 equivalent per mol of the carboxylic acid compound (1) or its reactive derivative. Here, mentioned as the reactive derivative are, for example, active ester derivatives, active amide derivatives and the like, usually used in the field of organic chemistry.

The amount of the amide formation reagent used varies depending on the kinds of a compound and a solvent used and other reaction conditions, and usually, is 1 to 50 equivalent, preferably 1 to 5 equivalent per mol of the carboxylic acid compound (1) or its reactive derivative.

The amount of the condensing aid used varies depending on the kinds of a compound and a solvent used and other reaction conditions, and usually, is 1 to 50 equivalent, preferably 1 to 5 equivalent per mol of the carboxylic acid compound (1) or its reactive derivative.

The amount of the base used varies depending on the kinds of a compound and a solvent used and other reaction conditions, and usually, is 1 to 50 equivalent, preferably 3 to 5 equivalent.

The reaction solvent used in this process is, for example, an inert organic solvent and is not particularly restricted providing it causes no disturbance in the reaction, and specific examples thereof include methylene chloride, chloroform, 1,2-dichloroethane, trichloroethane, N,N-dimethylformamide, acetic acid ethyl ester, acetic acid methyl ester, acetonitrile, benzene, xylene, toluene, 1,4-dioxane, tetrahydrofuran, dimethoxyethane or mixed solvents thereof, and from the standpoint of insurance of suitable reaction temperature, for example, methylene chloride, chloroform, 1,2-dichloroethane, acetonitrile, N,N-dimethylformamide and the like are particularly suitable.

The reaction temperature is from −100° C. to the boiling point of a solvent, preferably 0 to 30° C.

The reaction time is 0.5 to 96 hours, preferably 3 to 24 hours.

The base, amide formation reagent and condensing aid used in this process 1 can be used singly or in combination of two or more.

When the compound (3) has a protective group, this protective group can be removed appropriately. Removal of this protective group can be conducted by methods described in literatures (Protective Groups in Organic Synthesis, T. W. Green, second ed., John Wiley & Sons, 1991, and the like), methods according to these methods, or combinations of them with ordinary methods.

Thus obtained compound (3) can be isolated and purified by known separation purification means, for example, concentration, concentration under reduced pressure, crystallization, extraction with solvent, re-precipitation, chromatography and the like, or can be used in the subsequent process without isolation and purification.

(Process 2)

This process is a method of reacting an amide compound (3) obtained in the above-mentioned process 1 with a compound (4) to produced a compound (5).

In this reaction, a base may be added into the reaction system, if necessary. As the compound (4) used, phenols derivatives and thiol derivatives are preferable. As the phenols derivatives and thiol derivatives, for example, phenol, thiophenol, thioimidazole, thiotriazole and the like are mentioned. The amount of the compound (4) used varies depending on the kinds of a compound and a solvent used and other reaction conditions, and usually, is 2 to 50 equivalent, preferably 2 to 5 equivalent per mol of an amino derivative (3). Example of the base used include tertiary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undec-7-ene (DBU), 1,5-azabicyclo[4.3.0]non-5-ene (DBN) and the like; aromatic amines such as pyridine, 4-dimethylaminopyridine, picoline, lutidine, quinoline, isoquinoline and the like, alkali metal such as metal potassium, metal sodium, metal lithium and the like; alkali metal hydroxide such as sodium hydroxide, potassium hydroxide and the like, alkylated alkali metals such as butyl lithium and the like, alkali metal alkoxides such as potassium-tert-butylate, sodium ethylate, sodium methylate and the like, alkali metal hydroxides such as potassium hydroxide, sodium hydroxide and the like, and alkali metal carbonates such as potassium carbonate and the like, and of them, for example, tertiary aliphatic amines, alkali metal hydroxides and alkali metal carbonates are preferable, and particularly, triethylamine, N,N-diisopropylethylamine, sodium hydroxide and potassium carbonate are suitable.

The amount of the base used varies depending on the kinds of a compound and a solvent used and other reaction conditions, and usually, is 0 to 50 equivalent, preferably 2 to 10 equivalent per mol of an amino derivative (3). One or more bases can be used, if necessary.

The inert organic solvent used is not particularly restricted providing it causes no disturbance for the reaction, and specific examples thereof include methylene chloride, chloroform, 1,2-dichloroethane, trichloroethane, N,N-dimethylformamide, N,N-dimethylacetamide, acetic acid ethyl ester, acetic acid methyl ester, acetonitrile, benzene, xylene, water, toluene, 1,4-dioxane, tetrahydrofuran, or mixed solvents thereof.

Thus obtained compound (5) can be isolated and purified by known separation purification means, for example, concentration, concentration under reduced pressure, crystallization, extraction with solvent, re-precipitation, chromatography and the like.

(Process 3) This process is a method of reducing a compound (5) to produced a compound (I) used in the present invention. As the reducing reaction used in the present invention, methods well known to those skilled in the art can be used. As the reducing reaction used in this process, specifically mentioned are, for example, (1) a catalytic reduction method using hydrogen, formic acid, ammonium formate, hydrazine hydrate and palladium, platinum, nickel catalyst, (2) a reducing method using hydrochloric acid, ammonium chloride, (3) a reducing method using methanol and tin chloride, and other reducing methods.

The amount of the reducing agent used in the above-mentioned reducing reaction varies depending on the kinds of a compound and a solvent used and other reaction conditions, and usually, is 1 to 50 equivalent, preferably 2 to 20 equivalent per mol of a compound (5).

The reaction solvent used is not particularly restricted providing it causes no disturbance for the reaction, and for example, halogenated hydrocarbons such as dichloromethane, chloroform and the like, ethers such as diethyl ether, tert-butyl methyl ether, tetrahydrofuran and the like, amides such as N,N-dimethylformamide, N,N-dimethylacetamide and the like, sulfoxides such as dimethyl sulfoxide and the like, nitrites such as acetonitrile and the like, alcohols such as methanol, ethanol, propanol and the like, aromatic hydrocarbons such as benzene, toluene, xylene and the like, water or mixed solvents thereof can be used.

The reaction temperature and reaction time are not particularly restricted, and the reaction is conducted at a reaction temperature of about −10 to 100° C., preferably about 0 to 50° C. for 1 to 20 hours, preferably 1 to 5 hours.

Thus obtained compound (I) used in the present invention can be isolated and purified by known separation purification means, for example, concentration, concentration under reduced pressure, crystallization, extraction with solvent, re-precipitation, chromatography and the like, or can be used in the subsequent process without isolation and purification.

The above-mentioned compound in each process may have a protective group on each substituent. The protective group can be removed by known methods and methods according to them, or combinations of them with ordinary methods, appropriately in each process. Removal embodiments can be appropriately selected depending on the compound, the kind of the reaction and other reaction conditions, and when protective groups are removed separately, a case in which protective groups are removed simultaneously, and the like are envisaged, and embodiments can be appropriately selected by those skilled in the art. Listed as the protective group are, for example, protective groups for a hydroxyl group, protective groups for an amino group, protective groups for a carboxyl group, protective groups for an aldehyde, protective groups for a keto group, and the like. The order of removal of these protective groups is not particularly restricted.

The protective group for a hydroxyl group includes lower alkyl silyl groups such as a tert-butyldimethylsilyl group, tert-butyldiphenylsilyl group and the like, lower alkoxymethyl groups such as a methoxymethyl group, 2-methoxyethoxymethyl group and the like, aralkyl groups such as a benzyl group, p-methoxybenzyl group and the like, acyl groups such as a formyl group, acetyl group and the like, and of them, particularly, a tert-butyldimethylsilyl group, acetyl group and the like are preferable.

The protective group for an amino group includes aralkyl groups such as a benzyl group, p-nitrobenzyl group and the like, acyl groups such as a formyl group, acetyl group and the like, lower alkoxycarbonyl groups such as an ethoxycarbonyl group, tert-butoxycarbonyl group and the like, aralkyloxycarbonyl groups such as a benzyloxycarbonyl group, p-nitrobenzyloxycarbonyl group and the like, and of them, particularly, a nitrobenzyl group, tert-butoxycarbonyl group, benzyloxycarbonyl group and the like are preferable.

The protective group for a carboxyl group includes lower alkyl groups such as a methyl group, ethyl group, tert-butyl group and the like, aralkyl groups such as a benzyl group, p-methoxybenzyl group and the like, and of them, particularly, a methyl group, ethyl group, tert-butyl group, benzyl group and the like.

The protective group for a keto group includes a dimethyl ketal group, 1,3-dioxylane group, 1,3-dioxolane group, 1,3-dithiane group, 1,3-dithiolane group and the like, and of them, a dimethyl ketal group, 1,3-dioxolane group are more preferable.

The protective group for an aldehyde group includes a dimethyl acetal group, 1,3-dioxylane group, 1,3-dioxolane group, 1,3-dithiane group, 1,3-dithiolane group and the like, and of them, a dimethyl acetyl group, 1,3-dioxolane group and the like are more preferable.

In producing a compound used in the present invention, a protective group is introduced in some cases in a functional group for efficient progress of the reaction. Introduction of the protective group can be appropriately selected by those skilled in the art, and removal of the protective group can be conducted by methods such as Protective Groups in Organic Synthesis and the like, methods according to them, or combinations of them with ordinary methods. The order of removal of the protective group can also be appropriately selected by those skilled in the art.

Thus obtained compound (I) can be isolated and purified by known separation purification means, for example, concentration, concentration under reduced pressure, crystallization, re-precipitation, extraction with solvent, chromatography and the like, or can be used in the subsequent process without isolation and purification.

The compound (I) used in the present invention can be produced also by the following process. 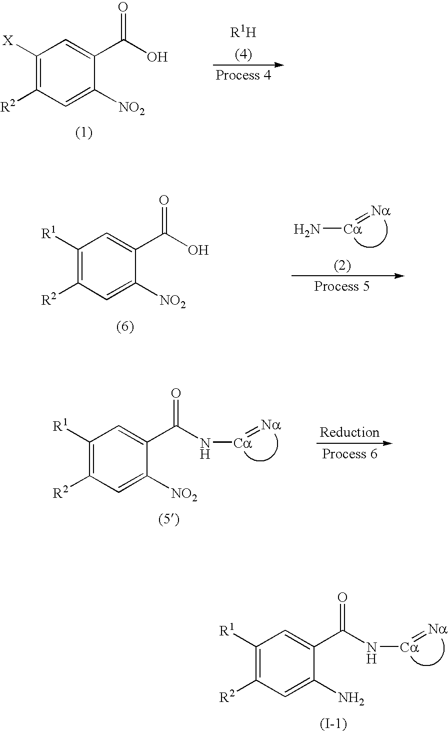
[wherein, marks are as defined above]

In the above-mentioned process 4, process 5 and process 6, the amount of a reagent, reaction solvent, reaction temperature and other reaction conditions can be the same as those in the above-mentioned process 1, process 2 and process 3.

When R² needs a protective group, the protective group can be appropriately selected by those skilled in the art by the above-mentioned methods such as Protective Groups in Organic Synthesis and the like, methods according to them, or combinations of them with ordinary methods.

Thus obtained compound (6), (5′) can be isolated and purified by known separation purification means, for example, concentration, concentration under reduced pressure, crystallization, re-precipitation, extraction with solvent and the like, or can be used in the subsequent process without isolation and purification.

The compound (I) used in the present invention can be isolated and purified by known separation purification means, for example, concentration, concentration under reduced pressure, crystallization, re-precipitation, extraction with solvent and the like.

In the above-mentioned processes 1 to 6, removal of the protective group can be conducted by Protective Groups in Organic Synthesis, T. W. Green, second ed., John Wiley & Sons, 1991, and the like, methods according to this, or combinations of them with ordinary methods, though varying depending on the kind of the protective group and the stability of a compound. For example, the removal can be conducted by solvolysis using an acid or base, chemical reduction using a hydrogenated metal complex and the like, or catalytic reduction using a palladium carbon catalyst, Raney nickel and the like.

The benzamide compound provided by the present invention can exist as a pharmaceutically acceptable salt. This salt can be produced by an ordinary method. Specifically, when the above-mentioned compound (I) has a basic group derived from an amino group, pyridyl group and the like in the molecule, this compound can be treated with an acid to be converted into a corresponding pharmaceutically acceptable salt.

Listed as the acid addition salt are, for example, hydrohalogenates such a hydrochloride, hydrofluoride, hydrobromide, hydroiodide and the like; inorganic acid salts such as nitrate, perchlorate, sulfate, phosphate, carbonate and the like; lower alkylsulfonates such as methanesulfonate, trifluoromethanesulfonate, ethanesulfonate and the like; arylsulfonates such as benzenesulfonate, p-toluenesulfonate and the like; organic acid salts such as fumarate, succinate, citrate, tartarate, oxalate, maleate and the like; acid addition salts which are organic acids such as amino acids such as glutamate, aspartate and the like. When the compound of the present invention has an acidic group in this group, for example, when it has a carboxyl group and the like, it can be converted into a corresponding pharmaceutically acceptable salt also by treating the compound with a base. As this base addition salt, for example, salts of alkali metals such as sodium, potassium and the like, salts of alkali earth metals such as calcium, magnesium and the like, ammonium salt, salts of organic bases such as guanidine, triethylamine, dicyclohexylamine and the like are mentioned. Further, the compound of the present invention may also be present as any hydrate or solvate of a free compound or salt thereof.

In the present invention, as described in examples, a crystal of a complex of a GK protein having an amino acid sequence as depicted in SEQ ID No. 5 with a compound of the above-mentioned formulae (IIIa) to (IIIc) is obtained. By analyzing the three dimensional structure coordinate of the crystal, it is clarified that, in a GK protein as depicted in SEQ ID No. 5, a compound binding portion is constituted of amino acid residues of tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459.

According to another embodiment of the present invention, there is provided a method of producing a crystal comprising a complex of a protein with a compound binding to the protein, comprising a protein production step of producing a protein having an amino acid sequence obtained by deletion of given number of amino acid residues at the N terminal side and/or C terminal side from a protein having an amino acid sequence as depicted in SEQ ID No. 2, and a step of reacting a compound binding to a protein obtained in the above-mentioned protein production step with a protein obtained in the above-mentioned protein production step.

The number of proteins produced in the above-mentioned protein production step is not particularly restricted providing there occurs no steric disturbance between an adjacent GK protein in a crystal. Specifically, for example, amino acid sequences obtained by deletion of 1 to 50, preferably 3 to 30, more preferably 5 to 25, further preferably 8 to 18, particularly preferably 11 to 15 amino acid resides at the N-terminal side, in an amino acid sequence as depicted in SEQ ID No. 2, and the like are listed. Amino acid sequences obtained by deletion of 1 to 8, preferably 1 to 7, more preferably 2 to 6 amino acid resides at the C-terminal side, and the like are listed.

(Drug Design Method Using Three Dimensional Structure Coordinate)

The three dimensional structure of a GK protein of the present invention obtained as described above is important information for a drug finding system by CARDD (Computer Aided Rational Drug Design). To clarify the active center and allosteric portion of this GK protein and to search a substance suitable for this portion and interacting with GK protein to disturb or activate the GK protein is an important step for drug development targeting a GK protein.

Namely, according to another embodiment of the present invention, there is provided a drug design method of designing the structure of a compound binding to a protein based on the steric structure information of the protein, wherein the steric structure information of the protein is information obtained by analyzing the crystal obtained as described above. As such a drug design method, there are a method of designing a drug using energy calculation, active prospective value analogous to this, or pharmacophore, and a method of visually designing a drug using a technology of computer graphics.

As the method using energy calculation, active prospective value analogous to this, or pharmacophore, there are exemplified (1) a drug design method comprising a connection portion guessing step of guessing a compound binding portion of the protein, based on steric structure information obtained as described above, and a selection step of selecting a compound adaptable to a compound binding portion guessed in the above-mentioned binding portion guessing step from a compound library, and (2) a binding portion guessing step of guessing a compound binding portion of the protein based on the above-mentioned steric structure information, and a compound structure fabricating step of fabricating the structure of a compound adaptable to a compound binding portion guessed in the above-mentioned binding portion guessing step.

As the method of guessing a compound binding portion of a protein, there is mentioned, for example, a method of estimating and specifying a portion to which a ligand is liable to connect, in a protein crystal structure relaxed without binding of a ligand, in addition to a method of visually confirming a portion of binding of a ligand in a co-crystal with a compound on a computer display. In each method, known methods and commercially available computer soft wares can be used. In the former method, for example, soft wares such as Insight II (Accelrys Inc.), SYBYL (Tripos Inc.), MOE (Chemical Computing Group) and the like can be used. On the other hand, in the latter method, for example, known method such as Cavity search: an algorithm for the isolation and display of cavity-like binding regions. (Journal of Computer-Aided Molecular Design. 4 (4): 337-54, 1990) and the like can be used, and the latter method can be carried out using softwares such as Site ID (Tripos Inc.) and the like.

When a binding portion on a protein with a compound can be guessed, compounds applicable to the guessed binding portion are selected. In this method of selecting compound candidates, structure information of compounds from an existing compound library is obtained, and the structure information of a compounds in the library is compared with the structure information of the binding portion guessed as described above, thus, bindable compound candidates are selected.

More specifically, the compound candidates are selected by judging whether conditions are satisfied or not while searching exhaustively the coordinate and orientation of each compound from a compound library, using, as search conditions, pharmacophores such as hydrogen bonding property or hydrophobicity and the like formed from one or more residues of amino acid residues (tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459) of an amino acid sequence as depicted in SEQ ID No. 5 or functional groups of ligands in a complex, and a protein surface made of a protein structure or a structure obtained by modifying the orientation of partial side chains.

In other alternative method, a candidate compound is virtually docked to the structure of a ligand binding portion constituted of amino acid residues (tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459) or a structure obtained by modifying the orientation of partial side chains, while searching exhaustively the coordinate and orientation of each compound from a compound library, and those forming a close interaction with one or more residues at a distance of 4 Å or less are selected from amino acid residues (tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459).

On the other hand, candidate compounds can also be selected by designing a bindable compound based on the structure information of a binding portion guessed as described above. More specifically, a compound structure is fabricated by variously connecting various atoms and functional groups so as to form an interaction with one or more residues, of the structure of a compound binding portion constituted of amino acid residues (tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459) of an amino acid sequence as depicted in SEQ ID No. 5 or a structure obtained by modifying the orientation of partial side chains. As this method, a method in which chemical groups such methyl, ethyl and the like are arranged at an active portion and suitable compounds are searched, and a method in which atoms are allowed to bind to an active portion using a computer program, are known.

As the method for energy evaluation by a computer, for example, there is a method in which bonding energy of a compound and a GK protein is obtained by molecular force field calculation. This calculation is applied to each compound in a database, and compound candidates capable of stably binding are selected from compounds in a library. Some computer programs such as Ludi of Insight II, and the like automatically select and output bindable compound candidates when the three dimensional structure coordinate of amino acid residues mutually acting in a protein molecule is imparted, and these programs can be suitably used.

Regarding drug design based on the three dimensional structure of a molecule, a lot of literatures including Iyakuhin no Kaihatsu, vol. 7, “Molecule Design”, (Hirokawa Shoten) are known. Specifically, first, a library (for example, about 150000 kinds) of lower molecule compounds (molecular weight: 1000 or less) can be screened, using a flexible ligand binding simulation software such as FlexiDock, Flex X and the like. Regarding chemical substances in this library, a three dimensional structure is constituted by a program such as CONCORD and the like, and compounds suitable for an active portion can be selected.

On the other hand, as the method of visually designing a drug, there is a drug design method comprising a binding portion guessing step of guessing a compound binding portion of the protein based on the steric structure information, and a design step of designing visually the structure of a compound so that a compound binding portion guessed in the above-mentioned binding portion guessing step and a compound adaptable to the compound binding portion interact. For example, structure fabrication or structure modification is visually conducted so as to form an interaction with one or more residues, of the structure of a ligand binding portion constituted of amino acid residues (tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459) of an amino acid sequence as depicted in SEQ ID No. 5 or a structure obtained by modifying the orientation of partial side chains.

Specifically, in a visual method, first, the structure of a crystal of a complex of a GK protein with a compound capable of binding with this is displayed on a screenof a computer according to the resulted structure coordinate. Bindabilities of compounds in a library with a GK protein are sequentially investigated while considering a chemical interaction on a computer. The chemical interaction to be considered here include an electrostatic interaction, hydrophobic interaction, hydrogen bond, van der Waals interaction and the like. Namely, a synthetic consideration is made whether the structure of the compound in a three dimensional space constitutes a preferable structure for interaction, so that a group liable to be negatively charged such as a carboxyl group, nitro group, halogen group and the like in the group of functional groups interacts with an amino acid residue having positive charge such as lysine, arginine, histidine of GK protein, so that a group liable to be positively charged such as an amino group, imino group, guanidyl group interacts with an amino acid residue having negative charge such as glutamic acid, aspartic acid of a GK protein, so that a hydrophobic functional group such as an aliphatic group and aromatic group interacts with a hydrophobic amino acid residue such as alanine, leucine, isoleucine, valine, proline, phenylalanine, tryptophane and methionine, so that a group correlated with a hydrogen bond such as a hydroxyl group, amide group and the like can form a hydrogen bond with a main chain or side chain portion of a GK protein, further, so that steric hindrance does not occur on binding of the compound with a GK protein, further, so that a void portion is filled to decrease a void portion as small as possible, and van der Waals interaction increases, and the like. Thus, factors such as an electrostatic interaction, hydrophobic interaction, van der Waals interaction, hydrogen bond and the like are synthetically considered visually on a computer screen, and judge whether a candidate compound can finally bind to a GK protein or not is made.

As the program for selecting compound candidates visually, simulation programs such as Insight II, MOE and the like are exemplified. For listing important candidates of compounds interacting with a GK protein, candidates compounds and GK protein are allowed to contact, and the activity of a GK protein is measured. Applicability of important candidate compounds is investigated by mixing important candidate compounds actually with a GK protein and crystallizing the mixture. Further, an applicable complex is modified by organic synthesis to give a more desired structure.

A visual means and a means considering energy can be appropriately combined and used. Such a computer software includes FlexiDock (Tripods Inc.), FlexX (Tripods Inc.), SYBYL (Tripods Inc.), Insight II (Accelrys Inc.), MOE (Chemical Computing Group Inc.) and the like.

In the present invention, compounds selected by the above-mentioned drug design methods are actually synthesized, and a group of these compounds can be provided as a compound array (or compound library). If such a compound array is used, a large amount of candidate compounds can be assayed in one time using a technology such as high throughput screening and the like, therefore, the activating agent or an inhibitor of a glucokinase can efficiently be screened.

(Compound Obtained by Method of the Present Invention and Therapeutic Compound Containing this)

Since a compound designed by the above-mentioned drug design methods has an ability of binding to a glucokinase, these compounds can be used as an activating compound for a glucokinase or a glucokinase inhibiting compound. Therapeutic agents or medical compositions containing such compounds can be effectively used as therapeutic agents correlated with a glucokinase activity (for example, diabetes treating agent).

The above-mentioned medical composition contains a pharmaceutically effective amount of a compound binding to a glucokinase of the present invention as an effective component, together with a suitable pharmaceutically acceptable carrier or diluent. As the pharmaceutically acceptable carrier which can be utilized in the above-mentioned medical composition (medical preparation), there are exemplified diluents and excipients such as a filler, extender, binding agent, wetting agent, disintegrator, surfactant, lubricant and the like, usually used corresponding to preparation use forms. These carriers are appropriately selected and used depending on the dose form of the resulted preparation.

Regarding the dose form of the medical composition of the present invention, various forms can be selected depending on treatment objects, and typical examples thereof include solid dose forms such as a tablet, pill, powder, pulvis, granule, capsule and the like, and liquid dose forms such as a solution, suspension, emulsion, syrup, elixir and the like, and these are further classified into an oral drug, parenteral drug, nasal drug, transvaginal drug, suppository, sublingual drug, ointment and the like depending on administration route, and can be blended, molded and prepared according to usual methods, respectively. For example, in molding into a tablet, examples of the above-mentioned preparation carriers capable of being used include excipients such as lactose, white sugar, sodium chloride, glucose, urea, starch, calcium carbonate, kaolin, crystalline cellulose, silic acid, potassium phosphate and the like, binding agents such as water, ethanol, propanol, simple syrup, glucose liquid, starch liquid, gelatin solution, carboxymethyl cellulose, hydroxypropyl cllulose, methyl cellulose, polyvinylpyrrolidone and the like, disintegrators such as carboxymethyl cellulose sodium, carboxymethyl cellulose calcium, low substitution hydroxypropyl cellulose, dry starch, sodium arginate, agar powder, laminaran powder, sodium hydrogen carbonate, calcium carbonate and the like, surfactants such as polyoxyethylenesorbitan fatty acid esters, sodium laurylsulfate, stearic mono-glyceride and the like, disintegration suppressing agents such as white sugar, stearin, cacao butter, hydrogenated oil and the like, absorption promoters such as quaternary ammonium base, sodium laurylsulfate and the like, wetting agents such as glycerin, starch and the like, adsorbents such as starch, lactose, kaolin, bentonite, colloidal silic acid and the like, lubricants such as purified talc, stearate, boric acid powder, polyethylene glycol and the like. Further, the tablet can be, if necessary, a tablet coated generally, for example, a sugar-coated tablet, gelatin-coated table, enteric coated table, film coated tablet, and can also be a double-layer tablet or multi-layer tablet.

In molding into a pill form, examples of preparation carriers capable of being used include excipients such as glucose, lactose, starch, cacao butter, hardened vegetable oil, kaolin, talc and the like, binding agents such as gum Arabic powder, tragacanth powder, gelatin, ethanol and the like, disintegrators such as laminaran, starch and the like.

The capsule is prepared by usually mixing effective components in the present invention with various preparation carries exemplified above according to ordinary methods and filling the mixture in a hard gelatin capsule, soft capsule and the like.

Liquid dose forms for oral administration include conventionally used inert diluents, for example, pharmaceutically acceptable solutions including water, and emulsion, suspension, syrup, elixir and the like, further, auxiliaries such as a wetting agent, emulsifier, suspending agent and the like are allowed to be contained, and these are prepared according to ordinary methods.

In preparing liquid dose forms for parenteral administration, for example, sterile aqueous or non-aqueous solutions, emulsion, suspension and the like, there can be used as a diluent, for example, water, ethyl alcohol, propylene glycol, polyethylene glycol, ethoxylated isostearyl alcohol, polyoxylated isostearyl alcohol, polyoxyethylene sorbitan fatty acid ester, olive oil and the like, and injectable organic esters, for example, ethyl oleate and the like can be compounded. To them, usual dissolution auxiliaries, buffers, wetting agents, emulsifiers, suspending agents, preservatives, dispersing agents and the like can be further added. Sterilization can be performed by a filtration operation through a bacteria holding filter, compounding of a bacteriocide, irradiation treatment and heating treatment and the like. These can also be prepared into a sterile solid composition form which can be dissolved in sterile water or suitable sterilizable medium directly before use.

In molding into a form of suppository and transvaginal preparation, examples of preparation carriers capable of being used include polyethylene glycol, cacao butter, higher alcohol, esters of higher alcohol, gelatin, semi-synthetic glyceride and the like.

In molding into a form of ointment such as paste, cream, gel and the like, examples of diluents capable of being used include white Vaseline, paraffin, glycerin, cellulose derivatives, propylene glycol, polyethylene glycol, silicon, ventonite and vegetable oils such as olive oil and the like.

Compositions for nasal or sublingual administration can be prepared according to ordinary methods using well-known standard excipients.

In the drug of the present invention, coloring agents, preservatives, aromatics, flavors, sweeteners and other medical products and the like are allowed to be contained, if necessary.

The amount and dosage of effective components to be contained in the above-mentioned medical preparations are not particularly restricted, and are appropriately selected widely depending on desired therapeutic affect, administration method, treatment period, age, sex and other conditions of patient, and the like. In general, the dose is usually advantageously about 0.01 mg to 1000 mg, preferably about 1 mg to 100 mg per day per body weight of 60 kg, and the drug can be administered once or in several time a day.

SEQ ID No. in the sequence list in this specification shows the following sequences.
[SEQ ID No. 1]

It shows a base sequence of DNA encoding a human-derived liver type glucokinase.
[SEQ ID No. 2]

It shows an amino acid sequence of a human-derived liver type glucokinase.
[SEQ ID No. 3]

It shows an amino acid sequence of a human-derived β-cell glucokinase.
[SEQ ID No. 4]

It shows a base sequence of DNA encoding a protein obtained by deletion of 11 amino acid residues at the N-terminal of a human-derived liver type glucokinase.
[SEQ ID No. 5]

It shows an amino acid sequence of a protein obtained by deletion of 11 amino acid residues at the N-terminal of a human-derived liver type glucokinase.
[SEQ ID No. 6]

It shows a base sequence of primer 1, used in a PCR reaction in the following Example 1.
[SEQ ID No. 7]

It shows a base sequence of primer 2, used in a PCR reaction in the following Example 1.
[SEQ ID No. 8]

It shows an amino acid sequence of a protein obtained by deletion of 15 amino acid residues at the N-terminal of a human-derived liver type glucokinase.
[SEQ ID No. 9]

It shows a base sequence of a primer, used in a PCR reaction in the following Example 6.
[SEQ ID No. 10]

It shows a base sequence of a primer, used in a PCR reaction in the following Example 6.

### EXAMPLES

The present invention will be illustrated specifically using examples.

(Method of Purification of Variant Enzyme)

Human glucokinases are classified into liver type glucokinases and pancreas type glucokinases depending on a difference in promoters, and they differ in 15 residues at the N-terminal. For crystallization intending analysis of a three dimensional structure, variant enzymes in which part or all of this part was deficient was produced in the following method.

Two kind of primer sets and cDNA of Human liver type glucokinase cloned on pCR2.1 (manufactured by INTROGEN)

PCR reactions were conducted using a combination of

(SEQ ID No. 6)

5′-gtcacaaggagccagaagcttatggccttgactctggtag-3′,
and

(SEQ ID No. 7)

5′-gaagccccacgacattgttcccttctgc-3,
and

(SEQ ID No. 9)

5′-ccaggcccagacagccaagcttatggtagagcagatcc-3′,
and

(SEQ ID No. 10)

5′-gaagccccacgacattgttcccttctgc-3′.

Hind III, ClaI fragment of the resulted PCR product was substituted with Hind III-Cla I region of liver type GK cloned on Hind III, Eco RI portion of pFLAG•CTC vector (Eastman Kodak), to obtain a cDNA coding a variant type GK (Δ1-11) having deletion of 1 to 11 residues of liver type GK and a variant type GK (Δ1-15) having deletion of 1 to 15 residues. The sequence of the resulted cDNA was recognized, then, E. coli DH5 α strain (manufactured by Takara Shuzo Co., Ltd.) was transformed using these vectors as a manifestation vector.

Transformants were cultivated on a LB medium until absorption at 600 nm reached 0.8, then, isopropyl-1-thio-β-D-galaxide (manufactured by Wako Pure Chemical Ltd.) was added to give a final concentration of 0.4 mM, and production of a protein was induced at 25° C. for 16 hours.

The cultivated E. coli was collected in a centrifugal separator, and suspended in a buffer containing the following components (50 mM potassium phosphate, pH 7.5, 50 mM NaCl, 2 mM DTT, 0.5 mM Pefabloc SC (manufactured by Kanto Kagaku K.K.), a proteinase inhibitor mixture (manufactured by Roche)).

The collected E. coli was crushed by an ultrasonic crushing method, and soluble fractions were dialyzed into the above-mentioned buffer, then, purified by a HiTrapQ column (manufactured by Amersham). The GK fraction eluted by gradient of potassium chloride by a HiTrapQ column was diluted to a salt concentration of 50 mM.

The diluted GK fraction was purified by a Glucosamin Sepharose column produced by a method shown in a literature (Preparative Biochemistry, 20 (2), 163-178 (1990)). The GK fraction was adsorbed on a Glucosamin Sepharose column, and impurities were removed by 100 mM sodium chloride, then, eluted by 1 M glucose.

The eluted GK fraction was purified by a MonoQ10/10 column. The GK fraction eluted by gradient of sodium chloride by a MonoQ10/10 column (manufactured by Amersham) was purified by a Superdex 200 column (manufactured by Amersham) using 50 mM Tris-Cl pH 7.2, 50 mM NaCl buffer as a mobile phase.

(Crystallization Method)

(Crystal of Variant Type GK (Δ1-11)/Glucose/Compound Complex)

The crystal of variant type GK (Δ1-11)/glucose/compound complex was obtained using a vapor diffusion means described below. The variant type GK (Δ1-11) means a glucokinase having an amino acid sequence as depicted in SEQ ID No. 5.

Namely, the variant type GK purified at high purity was concentrated, to obtain a solution of the variant type GK (25 mM Tris-Cl, 50 mM NaCl, 5 mM TCEP) having a final concentration of about 10 mg/ml. To this was added glucose of a final concentration of 20 mM and the following compound 1 (compound of the formula IIIa) having a final concentration of 0.3 mM activating GK, and used for crystallization. To 1 to 5 μl of a protein solution was added the equal amount of 28 to 30% PEG 1500, 0.1 M Hepes-NaOH (pH 6.6) as a crystallization solution and mixed to give a solution which was accommodated in a closed vessel containing 0.5 to 1 ml of a crystallization solution so that both solutions did not come into contact, and allowed to stand still at 20° C. After standing still for about 3 days to 1 month, a crystal having a maximum size of about 0.4 mm×0.4 mm×0.7 mm was obtained in the sample solution (Example 1).

Further, the crystal obtained by the above-mentioned method was immersed in 28 to 30% PEG 1500, 0.1 M Hepes-NaOH (pH 6.6) for about 3 to 7 days so that the following compound 2 (compound of the formula IIIb) was contained in a concentration of 0.3 mM, to obtain a complex crystal of the following compound 2 and the above-mentioned variant type GK (Example 2).

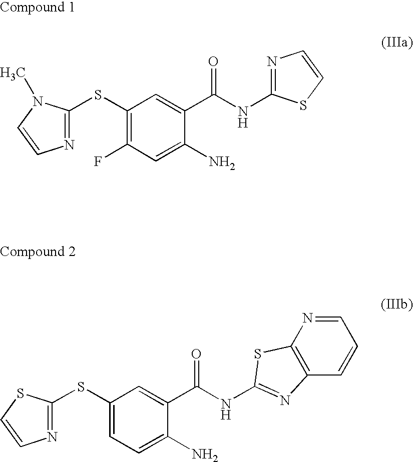

Crystallization was tried in the same manner as in Example 1 except that a compound 3 (compound of the formula IIIc) was used instead of the above-mentioned compound 1, as a result, the same crystal as in Example 1 was obtained (Example 3).

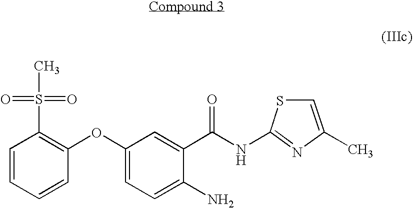

The resulted crystal was immersed in a crystallization solution containing 10% glycerol, subsequently, frozen quickly in liquid nitrogen. The X-ray diffraction data of the frozen crystal was collected in 100 K nitrogen flow by a vibration method in a synchrotron equipment KEK-PF, BL6B. From the resulted diffraction image, diffraction intensity was converted into numerical value using DENZO/SCALEPACK (manufactured by HKL), and a crystal structure factor was obtained. In this stage, the crystal was hexagonal and the space group had P6₅22 or P6₁22, and the unit lattice of the crystal was found to be, a=b=79.9 Å, c=322.2 Å, α=β=90°, γ=120°.

The structure was analyzed by conducting a molecule substitution method using the resulted structure factor and the three dimensional structure coordinate of Human hexakinase type 1. The calculation was conducted by an Amore program of CCP4 (Council for the Central laboratory of the Research Councils) using a data having a resolution ability of 8 Å to 4 Å. The R factor of the structure obtained by calculation was 53.7%, and the space group of the crystal was P6₅22 and it was found that one variant type GK molecule was contained at an asymmetric unit. An electron density map was obtained from this structure and structure factor, and the structure of the variant type glucokinase was determined using Program 0 (manufactured by Dat-ONO).

Next, preciseness of a position of an amino acid was increased using CNX (Accelrys Inc.), and identification of an amino acid residue was conducted using Program 0. This operation was repeated, and the structure coordinate of 448 amino acid residues from threonine 14 to cysteine 461 of the variant type glucokinase, one molecule of glucose, one molecule of compound A, one sodium ion, and 149 water molecules were identified, to determine the structure coordinate. The R factor which is an indicator of correctness of the finally determined structure was 23.2% for data of resolution ability from 30 Å to 2.3 Å, and the R factor (Rfree) for data not used in calculation in the stage of refining the structure was 27.4%. When confirmed by Ramachandran plot, an amino acid residue having an unacceptable structure was not found.

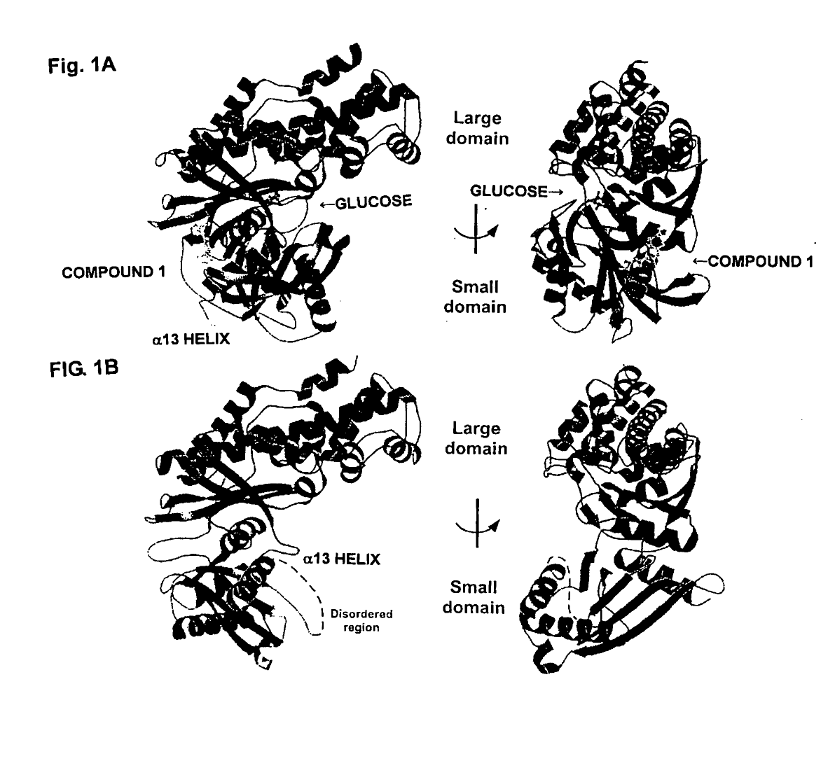

The determined structure of the variant type glucokinase was analogous to the structure of an isozyme, hexakinase, however, the structure of a portion to which a compound 1 (compound of the formula IIIa) that activates glucokinase binds was different. This difference in the structure cannot be prospected by capability of current computational chemistry, and this structure analysis clarified, for the first time, that this portion is an activator binding portion and, its detail steric structure. FIG. 1a is a ribbon view showing the three dimensional structure of glucokinase analyzed here. As shown in FIG. 1a, the newly found activator binding portion situated between a large domain and a small domain, and was away by about 20 Å from the active center of glucokinase to which a substrate glucokinase was bound. Amino acid residues of glucokinase constituting the activator binding portion were as described below. Tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459.

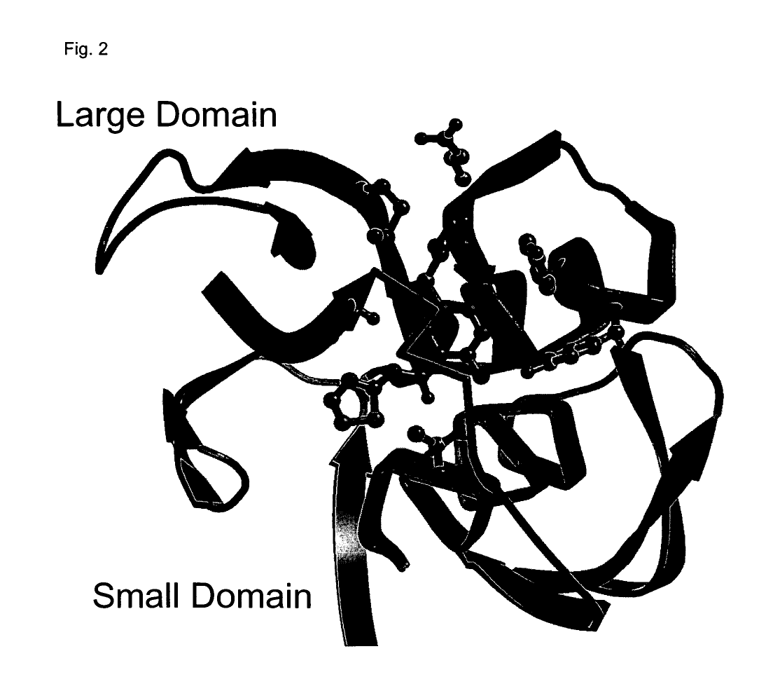

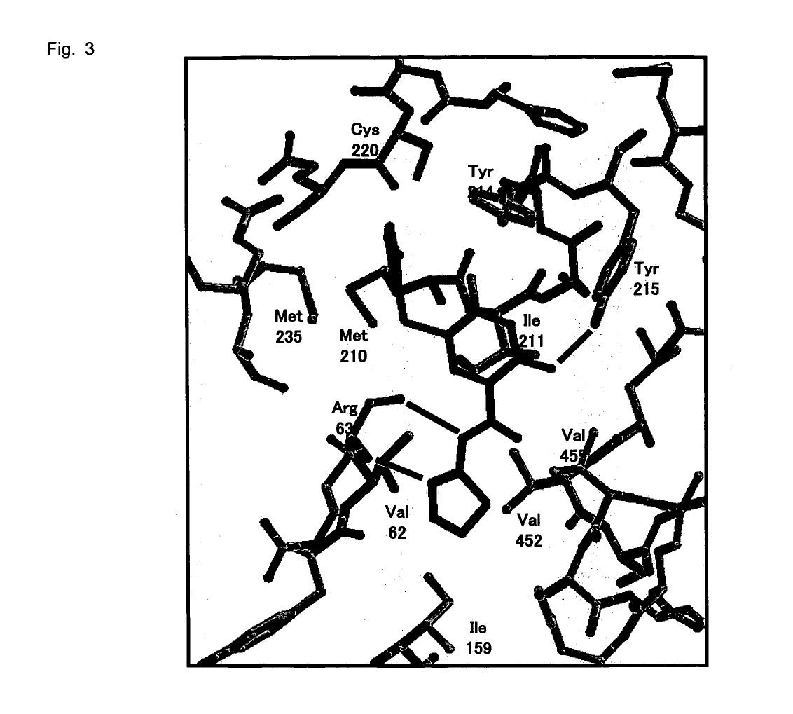

Binding mode of a compound 1 (compound of the formula IIIa) to this binding portion is shown in FIG. 2, and the structure of the binding portion of glucokinase is shown in FIG. 3. A thiazole ring was in van der Walls contact with molecules at amino acid side chains of valine 62, valine 452 and valine 455, and a nitrogen atom on a thiazole ring had a hydrogen bond with a nitrogen atom of a main chain of arginine 63. A nitrogen atom of an amide on the compound 1 had a hydrogen bond with an oxygen atom of a main chain of arginine 63. A benzene ring portion of the compound 1 was in van der Walls contact with isoleucine 211, and a fluorine atom substituted on a benzene ring was in van der Walls contact with a side chain of tyrosine 214. The aniline structure of the compound 1 formed a hydrogen bond with an oxygen atom of a side chain of tyrosine 215. An imidazole ring portion connected to a benzene ring via a sulfur was in van der Walls contact with amino acid side chain portions of methionine 210, methionine 235 and tyrosine 214. A portion of serine 64 to serine 69 connecting a large domain and a small domain projected into a solution, and the compound 1 was connected to a lower part of an arch structure formed by this portion (FIG. 3).

### Example 4

### Example of Drug Design

Library compounds were screened using, as research conditions, pharmacophore of a hydrogen bond acceptor and hydrogen bond donor generated respectively from NH and CO of a main chain of Arg63, hydrophobic pharmacophore formed in a space corresponding to a phenyl group of an aniline part of a ligand forming a complex, and the protein surface made based on the structure of a protein, by using a software UNITY (manufactured by Tripos), and the following compound 4 and compound 5 were obtained and assayed, to confirm activities of 780% and 560%. An activity of 780% means that when the activity of glucokinase is 100% in the case of control, the activity is enhanced up to 780% by these compound (using glucose 2.5 M, and ligand 10 μM).

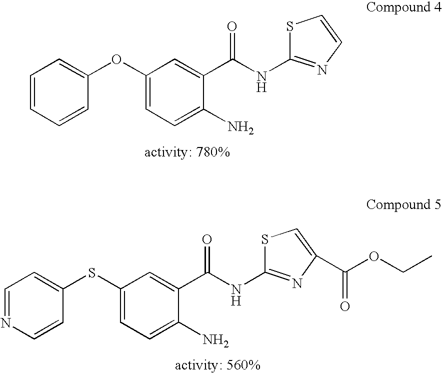

### Example 5

(Crystal of Variant Type GK (Δ1-15))

A crystal of a single entity of a variant type GK (Δ1-15)(glucokinase having an amino acid sequence as depicted in SEQ ID No. 8) was obtained by using a vapor diffusion means described below.

Namely, the variant type GK purified at high purity was concentrated, to obtain a solution of the variant type GK (25 mM Tris-Cl, pH 7.2, 50 mM NaCl, 5 mM TCEP) having a final concentration of about 10 mg/ml. To 1 to 5 μl of a protein solution was added the equal amount of a crystallization solution (1.5 to 1.6 M ammonium sulfate, 50 mM NaCl, 0.1 M Bicine NaOH (pH 8.7)) and mixed to give a solution which was accommodated in a sealed vessel containing 0.5 to 1 ml of a crystallization solution so that both solutions did not come into contact, and allowed to stand still at 20° C. After standing still for about 3 days to 1 month, a crystal having a maximum size of about 0.07 mm×0.07 mm×0.5 mm was obtained in the sample solution.

The resulted crystal was immersed in a crystallization solution containing 20% glycerol, subsequently, frozen quickly in liquid nitrogen. The X-ray diffraction data of the frozen crystal was collected in 100 K nitrogen flow by a vibration method in a synchrotron equipment Spring-8, BL32B2. From the resulted diffraction image, diffraction intensity was converted into numerical value using Mosflm, and a crystal structure factor was obtained. In this stage, the crystal was hexagonal and the space group had P6₅22 or P6₁22, and the unit lattice of the crystal was found to be, a=b=103.2 Å, c=281.0 Å, α=β=90°, γ=120°.

Next, the structure was analyzed by conducting a molecule substitution method using the resulted structure factor. As the steric structure model, three dimensional structure coordinates of respective domains of glucokinase determined by a crystal of variant type GK (Δ1-11)/glucose/compound complex were used separately. The calculation was conducted by an Amore program of CCP4 (Council for the Central laboratory of the Research Councils) using a data having a resolution of 8 Å to 4 Å. The space group of the crystal was P6₅22 and it was found that one variant type GK molecule GK (Δ1-15) was contained at an asymmetric unit. An electron density map was obtained from this structure and structure factor, and the structure of the variant type GK (Δ1-15) single entity was determined using Program 0 (manufactured by Dat-ONO).

Next, preciseness of a position of an amino acid was increased using CNX (manufactured by Molecular Simulation), and identification of an amino acid residue was conducted using Program 0. This operation was repeated, and the structure coordinate of 424 amino acid residues from methionine 15 to histidine 156 and from asparagine 180 to cysteine 461 of the variant type glucokinase, two sulfate ions, one sodium ion and 7 water molecules were identified, to determine the structure coordinate. The R factor which is an indicator of correctness of the finally determined structure was 23.8% for data of resolution from 50 Å to 3.4 Å, and the R factor (Rfree) for data not used in calculation in the stage of increase in preciseness of the structure was 30.6%. When confirmed by Ramachandran plot, an amino acid residue having an unacceptable structure was not found.

FIGS. 1a and 1b show, respectively, a ribbon view showing the structures of glucokinase (Δ1-11)/glucose/compound 1, and a ribbon view showing the structures of glucokinase (Δ1-15) single entity. In the determined structure of the variant type GK (Δ1-15), the structures of main parts of a large domain and a small domain were analogous to respective structures in the glucokinase determined by a crystal of variant type GK (Δ1-11)/glucose/compound complex, however, the relative positions of the two domains were different significantly. The main portion of a small domain in the structure of the variant type GK (Δ1-15) single entity was rotated by about 99° from the position of a small domain in the structure of variant type GK (Δ1-11)/glucose/compound complex. An α-13 helix constituting a small domain in the structure of variant type GK (Δ1-11)/glucose/compound complex situated at the C-terminal region of glucokinase did not constitute a small domain already in the structure of variant type GK (Δ1-15) single entity, and situated between both the domains. Further, since both of the substrate glucose binding portion and activating agent binding portion in the structure of variant type GK (Δ1-11)/glucose/compound complex were present between the two domains, the structures of these portions were changed significantly in newly determined structures. In the structure of variant type GK (Δ1-15) single entity, an amino acid residue performing an important role in enzymatic activity did not form an active portion, and the structure of variant type GK (Δ1-15) single entity was a structure of glucokinase inactivated condition. In the structure of variant type GK (Δ1-15) single entity, the activating agent binding portion disappeared completely. Change in the structure of glucokinase (rotation of domain by about 99°) observed by the structure of variant type GK (Δ1-11)/glucose/compound complex and the structure of variant type GK (Δ1-15) single entity was by far larger as compared with change in the structure (rotation of domain by about 12°) of hexokinase known to date, and cannot be prospected by the capacity of current computational chemistry, and it was clarified for the first time in this structure analysis.

It was also clarified that the activating agent for glucokinase can be designed by designing a compound that binds to a compound binding portion shown in the structure of variant type GK (Δ1-11)/glucose/compound complex, with an intention to inhibit structure change into the structure of variant type GK (Δ1-15) single entity which is of inactive type.

### INDUSTRIAL APPLICABILITY

As described above, according to the present invention, a crystal of a glucokinase protein which could not be easily crystallized conventionally can be obtained. A three dimensional structure coordinate obtained by analyzing the structure of this crystal can be used suitably for designing a compound that binds to glucokinase. Thus designed compound binds to glucokinase, therefore, it can be used as a therapeutic agent (for example, diabetes remedy) for patients correlated with glucokinase activity, as a glucokinase activating agent or inhibitor.

1. A glucokinase protein, used for crystallization.

2. The protein according to claim 1, consisting of an amino acid sequence as depicted in SEQ ID No. 5.

3. A crystal of a protein, consisting of an amino acid sequence as depicted in SEQ ID No. 5 or an amino acid sequence which is substantially the same as the amino acid sequence.

4. The crystal according to claim 3, wherein said protein is a glucokinase protein.

5. The crystal according to claim 3, which is a crystal of a protein having an amino acid sequence as depicted in SEQ ID No. 5.

6. The crystal according to claim 3, wherein the lattice constant satisfies the following formulae (1) to (4):
a=b=79.9±4 Å (1)
c=322.2±15 Å (2)
α=β=90° (3)
γ=120° (4)

7. The crystal according to claim 6, wherein the space group is P6₅22.

8. A crystal of a protein specified by three dimensional structure coordinate data described in Table 1.

9. A crystal wherein, in three dimensional structure coordinate data obtained by changing at least one datum in three dimensional structure coordinate data described in Table 1, the average square deviation between an atom (Cα atom) of a main chain of an amino acid shown by three dimensional structure coordinate data described in Table 1 and a Cα atom shown by said changed three dimensional structure coordinate data corresponding to the Cα atom is 0.6 Å or less.

10. The crystal according to claim 3, wherein the compound binding portion is constituted of at least one of amino acid residues of tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459, in an amino acid sequence as depicted in SEQ ID No. 5.

11. A crystal comprising a complex of a protein consisting of an amino acid sequence as depicted in SEQ ID No. 5 or an amino acid sequence which is substantially the same as the amino acid sequence with a compound capable of binding to the protein.

12. The crystal according to claim 11, wherein said compound is represented by the formula (I): 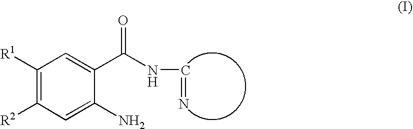
wherein, R¹ represents a halogen atom, —S—(O)ₚ-A, —S—(O)_{q}—B or —O—B (wherein, p and q are the same or different and represent an integer of 0 to 2, A represents a straight chain C₁ to C₆ alkyl group optionally substituted and B represents a 5-membered cyclic or 6-membered cyclic aryl group or heteroaryl group optionally substituted), R² represents a hydrogen atom or halogen atom, and 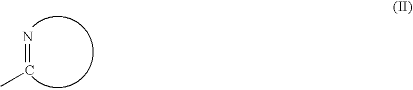
represents a mono-cyclic or di-cyclic heteroaryl group optionally substituted, having a nitrogen atom adjacent to a carbon atom connected to an amide group.

13. The crystal according to claim 12, wherein said compound is any of compounds of the formulae (IIIa) to (IIIc):

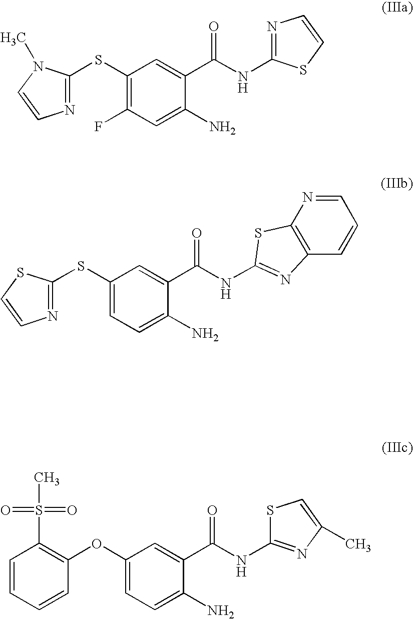

14. The protein according to claim 1, consisting of an amino acid sequence as depicted in SEQ ID No. 8.

15. A crystal of a protein, consisting of an amino acid sequence as depicted in SEQ ID No. 8 or an amino acid sequence which is substantially the same as the amino acid sequence.

16. The crystal according to claim 15, wherein said protein is a glucokinase protein.

17. The crystal according to claim 15, which is a crystal of a protein having an amino acid sequence as depicted in SEQ ID No. 8.

18. The crystal according to claim 15, wherein the lattice constant satisfies the following formulae:
a=b=103.2±5 Å (5)
c=281.0±7 Å (6)
α=β=90° (7)
γ=120° (8)

19. The crystal according to claim 18, wherein the space group is P6₅22.

20. A crystal of a protein specified by three dimensional structure coordinate data described in Table 2.

21. A crystal wherein, in three dimensional structure coordinate data obtained by changing at least one datum in three dimensional structure coordinate data described in Table 2, the average square deviation between an atom (Cα atom) of a main chain of an amino acid shown by three dimensional structure coordinate data described in Table 2 and a Cα atom shown by said changed three dimensional structure coordinate data corresponding to the Cα atom is 0.6 Å or less.

22. A method of producing a crystal comprising a complex of a protein with a compound binding to the protein, comprising
a protein production step of producing a protein having an amino acid sequence obtained by deleting 1 to 50 amino acid residues from one or both of the N terminal and C terminal of a protein having an amino acid sequence as depicted in SEQ ID No. 2, and
a protein reaction step of reacting a compound binding to a protein obtained in said protein production step with a protein obtained in said protein production step.

23. A method of producing a crystal of a protein, using a protein containing an amino acid sequence as depicted in SEQ ID No. 5 or an amino acid sequence which is substantially the same as the amino acid sequence, and having a glucokinase activity, and a compound capable of binding to said protein.

24. The method of producing a crystal of a protein according to claim 23, wherein said compound capable of binding to the protein is a compound of the formula (I): 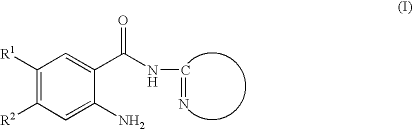
wherein, R¹ represents a halogen atom, —S—(O)ₚ-A, —S—(O)_{q}—B or —O—B (wherein, p and q are the same or different and represent an integer of 0 to 2, A represents a straight chain C₁ to C₆ alkyl group optionally substituted and B represents a 5-membered cyclic or 6-membered cyclic aryl group or heteroaryl group optionally substituted), R² represents a hydrogen atom or halogen atom, and 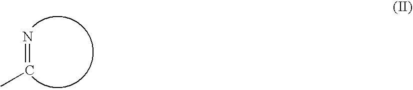
represents a mono-cyclic or di-cyclic heteroaryl group optionally substituted, having a nitrogen atom adjacent to a carbon atom connected to an amide group.

25. The method of producing a crystal according to claim 23, according to a co-crystallization method or soaking method.

26. A drug design method of designing the structure of a compound binding to a protein based on the steric structure information of the protein, wherein the steric structure information of the protein is information obtained by analyzing the crystal according to claim 3.

27. The drug design method according to claim 26, comprising a binding portion guessing step of guessing a compound binding portion of said protein based on said steric structure information, and
a selection step of selecting a compound adaptable to a compound binding portion guessed in said binding portion guessing step from a compound library.

28. The drug design method according to claim 26, comprising a binding portion guessing step of guessing a compound binding portion of said protein based on said steric structure information, and
a compound structure fabricating step of fabricating the structure of a compound adaptable to a compound binding portion guessed in said binding portion guessing step.

29. The drug design method according to claim 26, comprising a binding portion guessing step of guessing a compound binding portion of said protein based on said steric structure information, and
a design step of designing visually the structure of a compound so that a compound binding portion guessed in said binding portion guessing step and a compound adaptable to the compound binding portion interact.

30. The drug design method according to claim 26, wherein said compound binding portion is constituted of at least one of amino acid residues of tyrosine 61 to serine 69, glutamic acid 96 to glutamine 98, isoleucine 159, methionine 210 to tyrosine 215, histidine 218 to glutamic acid 221, methionine 235, arginine 250 and leucine 451 to lysine 459, in an amino acid sequence as depicted in SEQ ID No. 5.

31. The drug design method according to claim 26, further comprising a step of measuring the physiological activity of a candidate compound guessed to be adaptable to said compound binding portion.

32. The drug design method according to claim 26, further comprising a binding judgment step of contacting a candidate compound guessed to be adaptable to said compound binding portion with a protein containing an amino acid sequence as depicted in SEQ ID No. 5 or an amino acid sequence which is substantially the same as the amino acid sequence, and judging if the candidate compound binds to the protein or not.

33. A method of producing a compound array, comprising combining compounds in a group selected by the drug design method according to claim 26, as a compound array.
